(19) United States
(12) Patent Application Publication
(10) Pub. No.: US 2021/0395241 A1
(43) Pub. Date: Dec. 23, 2021

(54) NLRP MODULATORS

(71) Applicant: Novartis AG, Basel (CH)

(72) Inventors: Gary GLICK, Ann Arbor, MI (US); William R. Roush, Boston, MA (US); Shankar VENKATRAMAN, Lansdale, PA (US); Dong-Ming SHEN, Edison, NJ (US); Shomir GHOSH, Brookline, MA (US); Hans Martin SEIDEL, Concord, MA (US); Luigi FRANCHI, Ann Arbor, MI (US); David Guenther WINKLER, Arlington, MA (US); Anthony William OPIPARI, Dexter, MI (US); Jason KATZ, Newton, MA (US)

(21) Appl. No.: 17/252,613

(22) PCT Filed: Jul. 2, 2019
(86) PCT No.: PCT/US2019/040395

§ 371 Date: Dec. 15, 2020

Related U.S. Application Data
(60) Provisional application No. 62/693,840, filed on Jul. 3, 2018.
(60) Provisional application No. 62/812,563, filed on Mar. 1, 2019.

(51) Int. Cl. C07D 417/12; C07D 277/36; C07C 311/51; A61K 45/06

(52) U.S. Cl. CPC C07D 417/12; A61K 45/06; C07C 311/51; C07D 277/36

(57) ABSTRACT

In one aspect, compounds of Formula AA, or a pharmaceutically acceptable salt thereof, are featured: or a pharmaceutically acceptable salt thereof, wherein the variables shown in Formula A can be as defined anywhere herein.

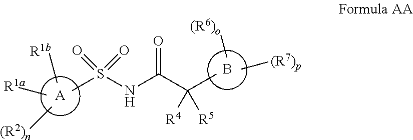

### TECHNICAL FIELD

This disclosure features chemical entities (e.g., a compound that modulates (e.g., antagonizes) NLRP3, or a pharmaceutically acceptable salt, and/or hydrate, and/or cocrystal, and/or drug combination of the compound) that are useful, e.g., for treating a condition, disease or disorder in which a decrease or increase in NLRP3 activity (e.g., an increase, e.g., a condition, disease or disorder associated with NLRP3 signaling) contributes to the pathology and/or symptoms and/or progression of the condition, disease or disorder in a subject (e.g., a human). This disclosure also features compositions as well as other methods of using and making the same.

### BACKGROUND

The NLRP3 inflammasome is a component of the inflammatory process and its aberrant activation is pathogenic in inherited disorders such as the cryopyrin associated periodic syndromes (CAPS). The inherited CAPS Muckle-Wells syndrome (MWS), familial cold autoinflammatory syndrome (FCAS) and neonatal onset multi-system inflammatory disease (NOMID) are examples of indications that have been reported to be associated with gain of function mutations in NLRP3.

NLRP3 can form a complex and has been implicated in the pathogenesis of a number of complex diseases, including but not limited to metabolic disorders such as type 2 diabetes, atherosclerosis, obesity and gout, as well as diseases of the central nervous system, such as Alzheimer's disease and multiple sclerosis and Amyotrophic Lateral Sclerosis and Parkinson disease, lung disease, such as asthma and COPD and pulmonary idiopathic fibrosis, liver disease, such as NASH syndrome, viral hepatitis and cirrhosis, pancreatic disease, such as acute and chronic pancreatitis, kidney disease, such as acute and chronic kidney injury, intestinal disease such as Crohn's disease and Ulcerative Colitis, skin disease such as psoriasis, musculoskeletal disease such as scleroderma, vessel disorders, such as giant cell arteritis, disorders of the bones, such as Osteoarthritis, osteoporosis and osteopetrosis disorders eye disease, such as glaucoma and macular degeneration, diseased caused by viral infection such as HIV and AIDS, autoimmune disease such as Rheumatoid Arthritis, Systemic Lupus Erythematosus, Autoimmune Thyroiditis, Addison's disease, pernicious anemia, cancer and aging.

In light of the above, it would be desirable to provide compounds that modulate (e.g., antagonize) NLRP3.

### SUMMARY

This disclosure features chemical entities (e.g., a compound that modulates (e.g., antagonizes) NLRP3, or a pharmaceutically acceptable salt, and/or hydrate, and/or cocrystal, and/or drug combination of the compound) that are useful, e.g., for treating a condition, disease or disorder in which a decrease or increase in NLRP3 activity (e.g., an increase, e.g., a condition, disease or disorder associated with NLRP3 signaling).

In some embodiments, provided herein is a compound of Formula AA

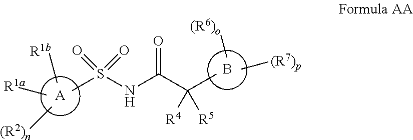

or a pharmaceutically acceptable salt thereof, wherein the variables in Formula AA can be as defined anywhere herein.

This disclosure also features compositions as well as other methods of using and making the same.

An “antagonist” of NLRP3 includes compounds that inhibit the ability of NLRP3 to induce the production of IL-1β and/or IL-18 by directly binding to NLRP3, or by inactivating, destabilizing, altering distribution, of NLRP3 or otherwise.

In one aspect, pharmaceutical compositions are featured that include a chemical entity described herein (e.g., a compound described generically or specifically herein or a pharmaceutically acceptable salt thereof or compositions containing the same) and one or more pharmaceutically acceptable excipients.

In one aspect, methods for modulating (e.g., agonizing, partially agonizing, antagonizing) NLRP3 activity are featured that include contacting NLRP3 with a chemical entity described herein (e.g., a compound described generically or specifically herein or a pharmaceutically acceptable salt thereof or compositions containing the same). Methods include in vitro methods, e.g., contacting a sample that includes one or more cells comprising NLRP3, as well as in vivo methods.

In a further aspect, methods of treatment of a disease in which NLRP3 signaling contributes to the pathology and/or symptoms and/or progression of the disease are featured that include administering to a subject in need of such treatment an effective amount of a chemical entity described herein (e.g., a compound described generically or specifically herein or a pharmaceutically acceptable salt thereof or compositions containing the same).

In a further aspect, methods of treatment are featured that include administering to a subject a chemical entity described herein (e.g., a compound described generically or specifically herein or a pharmaceutically acceptable salt thereof or compositions containing the same), wherein the chemical entity is administered in an amount effective to treat a disease in which NLRP3 signaling contributes to the pathology and/or symptoms and/or progression of the disease, thereby treating the disease.

Embodiments can include one or more of the following features.

The chemical entity can be administered in combination with one or more additional therapies with one or more agents suitable for the treatment of the condition, disease or disorder.

Examples of the indications that may be treated by the compounds disclosed herein include but are not limited to metabolic disorders such as type 2 diabetes, atherosclerosis, obesity and gout, as well as diseases of the central nervous system, such as Alzheimer's disease and multiple sclerosis and Amyotrophic Lateral Sclerosis and Parkinson disease, lung disease, such as asthma and COPD and pulmonary idiopathic fibrosis, liver disease, such as NASH syndrome, viral hepatitis and cirrhosis, pancreatic disease, such as acute and chronic pancreatitis, kidney disease, such as acute and chronic kidney injury, intestinal disease such as Crohn's disease and Ulcerative Colitis, skin disease such as psoriasis, musculoskeletal disease such as scleroderma, vessel disorders, such as giant cell arteritis, disorders of the bones, such as osteoarthritis, osteoporosis and osteopetrosis disorders, eye disease, such as glaucoma and macular degeneration, diseases caused by viral infection such as HIV and AIDS, autoimmune disease such as rheumatoid arthritis, systemic Lupus erythematosus, autoimmune thyroiditis; Addison's disease, pernicious anemia, cancer and aging.

The methods can further include identifying the subject.

Other embodiments include those described in the Detailed Description and/or in the claims.

### Additional Definitions

To facilitate understanding of the disclosure set forth herein, a number of additional terms are defined below. Generally, the nomenclature used herein and the laboratory procedures in organic chemistry, medicinal chemistry, and pharmacology described herein are those well-known and commonly employed in the art. Unless defined otherwise, all technical and scientific terms used herein generally have the same meaning as commonly understood by one of ordinary skill in the art to which this disclosure belongs. Each of the patents, applications, published applications, and other publications that are mentioned throughout the specification and the attached appendices are incorporated herein by reference in their entireties.

As used herein, the term “NLRP3” is meant to include, without limitation, nucleic acids, polynucleotides, oligonucleotides, sense and antisense polynucleotide strands, complementary sequences, peptides, polypeptides, proteins, homologous and/or orthologous NLRP3 molecules, isoforms, precursors, mutants, variants, derivatives, splice variants, alleles, different species, and active fragments thereof.

The term “acceptable” with respect to a formulation, composition or ingredient, as used herein, means having no persistent detrimental effect on the general health of the subject being treated.

“API” refers to an active pharmaceutical ingredient.

The terms “effective amount” or “therapeutically effective amount,” as used herein, refer to a sufficient amount of a chemical entity (e.g., a compound exhibiting activity as a modulator of NLRP3, or a pharmaceutically acceptable salt and/or hydrate and/or cocrystal thereof;) being administered which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is determined using any suitable technique, such as a dose escalation study.

The term “excipient” or “pharmaceutically acceptable excipient” means a pharmaceutically-acceptable material, composition, or vehicle, such as a liquid or solid filler, diluent, carrier, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington; The Science and Practice of Pharmacy, 21 st ed.; Lippincott Williams & Wilkins: Philadelphia, Pa., 2005; Handbook of Pharmaceutical Excipients, 6th ed.; Rowe et al, Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

The term “pharmaceutically acceptable salt” may refer to pharmaceutically acceptable addition salts prepared from pharmaceutically acceptable non-toxic acids including inorganic and organic acids. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. The term “pharmaceutically acceptable salt” may also refer to pharmaceutically acceptable addition salts prepared by reacting a compound having an acidic group with a base to form a salt such as an ammonium salt, an alkali metal salt, such as a sodium or a potassium salt, an alkaline earth metal salt, such as a calcium or a magnesium salt, a salt of organic bases such as dicyclohexylamine, A-methyl-D-glucamine, tris(hydroxymethyl)methylamine, and salts with amino acids such as arginine, lysine, and the like, or by other methods previously determined. The pharmacologically acceptable salt s not specifically limited as far as it can be used in medicaments. Examples of a salt that the compounds described hereinform with a base include the following: salts thereof with inorganic bases such as sodium, potassium, magnesium, calcium, and aluminum; salts thereof with organic bases such as methylamine, ethylamine and ethanolamine; salts thereof with basic amino acids such as lysine and ornithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid:organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

The term “pharmaceutical composition” refers to a mixture of a compound described herein with other chemical components (referred to collectively herein as “excipients”), such as carriers, stabilizers, diluents, dispersing agents, suspending agents, and/or thickening agents. The pharmaceutical composition facilitates administration of the compound to an organism. Multiple techniques of administering a compound exist in the art including, but not limited to: rectal, oral, intravenous, aerosol, parenteral, ophthalmic, pulmonary, and topical administration.

The term “subject” refers to an animal, including, but not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human.

The terms “treat,” “treating,” and “treatment,” in the context of treating a disease or disorder, are meant to include alleviating or abrogating a disorder, disease, or condition, or one or more of the symptoms associated with the disorder, disease, or condition; or to slowing the progression, spread or worsening of a disease, disorder or condition or of one or more symptoms thereof.

The terms “hydrogen” and “H” are used interchangeably herein.

The term “halo” refers to fluoro (F), chloro (Cl), bromo (Br), or iodo (I).

The term “alkyl” refers to a hydrocarbon chain that may be a straight chain or branched chain, saturated or unsaturated, containing the indicated number of carbon atoms. For example, C₁₋₁₀ indicates that the group may have from 1 to 10 (inclusive) carbon atoms in it. Non-limiting examples include methyl, ethyl, iso-propyl, tert-butyl, n-hexyl.

The term “haloalkyl” refers to an alkyl, in which one or more hydrogen atoms is/are replaced with an independently selected halo.

The term “alkoxy” refers to an —O-alkyl radical (e.g., —OCH₃).

The term “carbocyclic ring” as used herein includes an aromatic or nonaromatic cyclic hydrocarbon group having 3 to 10 carbons, such as 3 to 8 carbons, such as 3 to 7 carbons, which may be optionally substituted. Examples of carbocyclic rings include five-membered, six-membered, and seven-membered carbocyclic rings.

The term “heterocyclic ring” refers to an aromatic or nonaromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2, or 3 atoms of each ring may be substituted by a substituent. Examples of heterocyclic rings include five-membered, six-membered, and seven-membered heterocyclic rings.

The term “cycloalkyl” as used herein includes an nonaromatic cyclic, bicylic, fused, or spiro hydrocarbon radical having 3 to 10 carbons, such as 3 to 8 carbons, such as 3 to 7 carbons, wherein the cycloalkyl group which may be optionally substituted. Examples of cycloalkyls include five-membered, six-membered, and seven-membered rings. Examples include cyclopropyl, cyclobutyl, cyclopentyl, cyclopentenyl, cyclohexyl, cyclohexenyl, cycloheptyl, and cyclooctyl.

The term “heterocycloalkyl” refers to an nonaromatic 5-8 membered monocyclic, 8-12 membered bicyclic, or 11-14 membered tricyclic ring, fused, or spiro system radical having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2, or 3 atoms of each ring may be substituted by a substituent. Examples of heterocycloalkyls include five-membered, six-membered, and seven-membered heterocyclic rings. Examples include piperazinyl, pyrrolidinyl, dioxanyl, morpholinyl, tetrahydrofuranyl, and the like.

The term “aryl” is intended to mean an aromatic ring radical containing 6 to 10 ring carbons. Examples include phenyl and naphthyl.

The term “heteroaryl” is intended to mean an aromatic ring system containing 5 to 14 aromatic ring atoms that may be a single ring, two fused rings or three fused rings wherein at least one aromatic ring atom is a heteroatom selected from, but not limited to, the group consisting of O, S and N. Examples include furanyl, thienyl, pyrrolyl, imidazolyl, oxazolyl, thiazolyl, isoxazolyl, pyrazolyl, isothiazolyl, oxadiazolyl, triazolyl, thiadiazolyl, pyridinyl, pyrazinyl, pyrimidinyl, pyridazinyl, triazinyl and the like. Examples also include carbazolyl, quinolizinyl, quinolinyl, isoquinolinyl, cinnolinyl, phthalazinyl, quinazolinyl, quinoxalinyl, triazinyl, indolyl, isoindolyl, indazolyl, indolizinyl, purinyl, naphthyridinyl, pteridinyl, carbazolyl, acridinyl. phenazinyl, phenothiazinyl, phenoxazinyl, benzoxazolyl, benzothiazolyl, 1H-benzimidazolyl, imidazopyridinyl, benzothienyl, benzofuranyl, isobenzofuran and the like.

The term “hydroxy” refers to an OH group.

The term “amino” refers to an NH₂ group.

The term “oxo” refers to O. By way of example, substitution of a CH₂ a group with oxo gives a C═O group.

As used herein, the terms “the ring A” or “A” are used interchangeably to denote

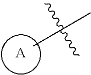

in formula AA, wherein the bond that is shown as being broken by the wavy line [symbol] connects A to the S(O)(NHR³)═N moiety of Formula AA.

As used herein, the terms “the ring B” or “B” are used interchangeably to denote

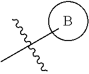

in formula AA wherein the bond that is shown as being broken by the wavy line [symbol] connects B to the CR⁴R⁵ group of Formula AA.

As used herein, the term “the substituted ring A” is used to denote

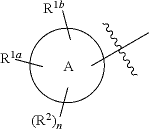

in formula AA, wherein the bond that is shown as being broken by the wavy line [symbol] connects A to the S(O₂)NH moiety of Formula AA.

As used herein, the term “the optionally substituted ring B” is used to denote

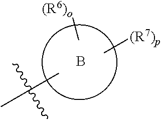

in formula AA, wherein the bond that is shown as being broken by the wavy line [symbol] connects B to the CR⁴R⁵ group of Formula AA.

As used herein, the recitation “S(O₂)”, alone or as part of a larger recitation, refers to the group

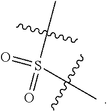 [SMILES: *S(*)(=O)=O]

In addition, atoms making up the compounds of the present embodiments are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

In addition, by way of example, a compound that is represented as containing the moiety

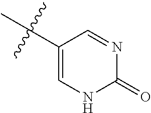 [SMILES: *c1cnc(=O)[nH]c1]

is also intended to include the tautomeric form containing the moiety

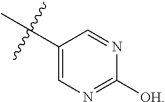 [SMILES: *c1cnc(O)nc1]

The details of one or more embodiments of the invention are set forth in the accompanying drawings and the description below. Other features and advantages of the invention will be apparent from the description and drawings, and from the claims.

### DETAILED DESCRIPTION

In some embodiments, provided herein is a compound of Formula AA

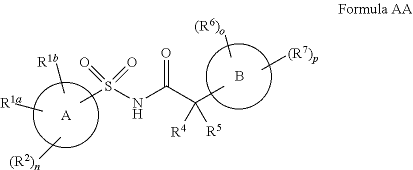

wherein
n=0 or 1;
o=1 or 2;
p=0, 1, 2, or 3;
wherein
A is a 5- to 10-membered heteroaryl or a C₆-C₁₀ aryl;
B is a 5-10-membered heteroaryl or a C₆-C₁₀ aryl;
wherein
R^{1a} is a C₁-C₆ alkyl, —CR¹¹R¹²NR¹¹R¹², or —SO₂NR¹¹R¹²;

wherein the C₁-C₆ alkyl is substituted with one or more hydroxy or —OSi(R¹³)₃; R^{1b} is a C₁-C₆ alkyl substituted with one or more hydroxy, —SO₂NR¹¹R¹², —SO₂R¹³, —CONR¹¹R¹², —OR¹¹, —COR¹³, —CO₂R¹³, —NR¹³CONR¹¹R¹²; —CR¹¹R¹²CN, —NR¹¹SO₂R¹³, —NR¹¹CONR¹¹R¹², —CR¹¹R¹²NR¹¹R¹², CN, and —NR¹¹COR¹²;

at least one R⁶ is ortho to the bond connecting the B ring to the CR⁴R⁵ group of Formula AA; R² is selected from C₁-C₆ alkyl, C₁-C₆ haloalkyl, C₁-C₆ alkoxy, C₁-C₆ haloalkoxy, halo, CN, NO₂, COC₁-C₆ alkyl, CO—C₆-C₁₀ aryl, CO-(5- to 10-membered heteroaryl), CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, NHCOOC₁-C₆ alkyl, NH—(C═NR¹³)NR¹¹R¹², CONR⁸R⁹, SF₅, SC₁-C₆ alkyl, S(O₂)C₁-C₆ alkyl, S(O₂)NR¹¹R¹², S(O)C₁-C₆ alkyl, C₃-C₇ cycloalkyl, and 3- to 7-membered heterocycloalkyl, wherein the C₁-C₆ alkyl, C₁-C₆ haloalkyl, C₃-C₇ cycloalkyl, and 3- to 7-membered heterocycloalkyl is optionally substituted with one or more substituents each independently selected from hydroxy, halo, CN, oxo, C₁-C₆ alkyl, C₁-C₆ alkoxy, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), and OCO(3- to 7-membered heterocycloalkyl);
wherein each C₁-C₆ alkyl substituent and each C₁-C₆, alkoxy substituent of the R² C₃-C₇ cycloalkyl or of the R² 3- to 7-membered heterocycloalkyl is further optionally independently substituted with one to three hydroxy, halo, or oxo;
wherein the 3- to 7-membered heterocycloalkyl, C₆-C₁₀ aryl, and 5- to 10-membered heteroaryl of the R² C₁-C₆ alkyl, the R² C₁-C₆ haloalkyl, the R² C₃-C₇ cycloalkyl, or the R² 3- to 7-membered heterocycloalkyl are optionally substituted with one or more substituents independently selected from halo, C₁-C₆ alkyl, and OC₁-C₆ alkyl;
   R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₁₀ cycloalkyl, 3- to 10-membered heterocycloalkyl, and C₂-C₆ alkenyl,
   wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, O(C₃-C₁₀ cycloalkyl), and S(O₂)C₁-C₆ alkyl; and wherein the C₁-C₆ alkyl or C₁-C₆ alkoxy that R⁶ or R⁷ is substituted with is optionally substituted with one or more hydroxyl, halo, C₆-C₁₀ aryl or NR⁸R⁹, or wherein R⁶ or R⁷ is optionally fused to a five-to-seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹; each of R⁴ and R⁵ is independently selected from hydrogen and C₁-C₆ alkyl;
   R¹⁰ is C₁-C₆ alkyl;
   each of R⁸ and R⁹ at each occurrence is independently selected from hydrogen, C₁-C₆ alkyl, (C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or
   R⁸ and R⁹ taken together with the nitrogen they are attached to form a 3- to 7-membered ring optionally containing one or more heteroatoms in addition to the nitrogen they are attached to;
   R¹³ is C₁-C₆ alkyl, C₆-C₁₀ aryl, or 5- to 10-membered heteroaryl; and each of R¹¹ and R¹² at each occurrence is independently selected from hydrogen and C₁-C₆ alkyl optionally substituted with hydroxy;
   with the proviso that the compound of Formula AA is not a compound selected from the group consisting of:

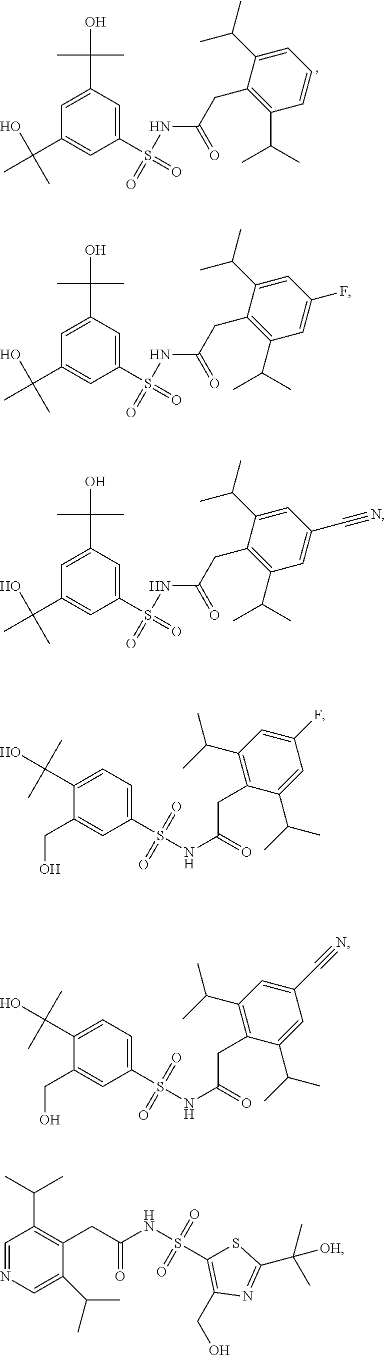 [SMILES: CC(C)c1cccc(C(C)C)c1CC(=O)NS(=O)(=O)c1cc(C(C)(C)O)cc(C(C)(C)O)c1 ; CC(C)c1cc(F)cc(C(C)C)c1CC(=O)NS(=O)(=O)c1cc(C(C)(C)O)cc(C(C)(C)O)c1 ; CC(C)c1cc(C#N)cc(C(C)C)c1CC(=O)NS(=O)(=O)c1cc(C(C)(C)O)cc(C(C)(C)O)c1 ; CC(C)c1cc(F)cc(C(C)C)c1CC(=O)NS(=O)(=O)c1ccc(C(C)(C)O)c(CO)c1 ; CC(C)c1cc(C#N)cc(C(C)C)c1CC(=O)NS(=O)(=O)c1ccc(C(C)(C)O)c(CO)c1 ; CC(C)c1cncc(C(C)C)c1CC(=O)NS(=O)(=O)c1sc(C(C)(C)O)nc1CO]

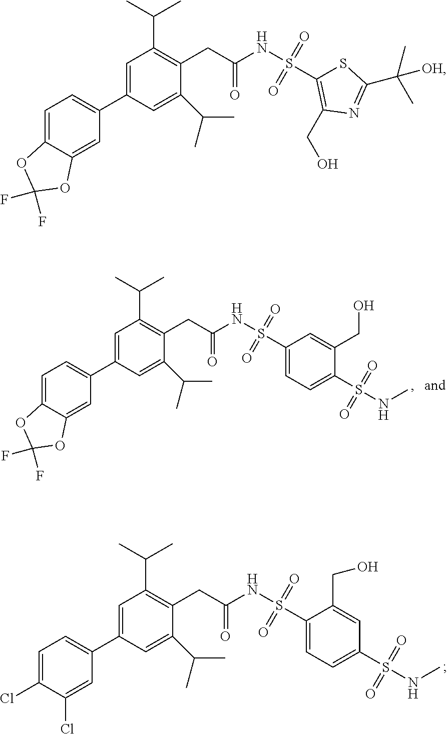 [SMILES: CC(C)c1cc(-c2ccc3c(c2)OC(F)(F)O3)cc(C(C)C)c1CC(=O)NS(=O)(=O)c1sc(C(C)(C)O)nc1CO ; CNS(=O)(=O)c1ccc(S(=O)(=O)NC(=O)Cc2c(C(C)C)cc(-c3ccc4c(c3)OC(F)(F)O4)cc2C(C)C)cc1CO ; CNS(=O)(=O)c1ccc(S(=O)(=O)NC(=O)Cc2c(C(C)C)cc(-c3ccc(Cl)c(Cl)c3)cc2C(C)C)c(CO)c1]

or a pharmaceutically acceptable salt thereof.

Without being bound by theory, it is believed that the presence of the two substituents R^{1a} and R^{1b} result in compounds that cross the intestinal barrier in a limited manner and are therefore result in compounds that are restricted to the gut and provide targeted delivery to the gut. Applicants have surprisingly found that the presence of at least two substituents, and particularly two polar substituents R^{1a} and R^{1b} provide compounds of formula AA that are poorly absorbed into systemic circulation after oral administration and are therefore restricted to the gut. Without being bound by theory, it is further hypothesized that the gut restricted compounds of the present invention can be used for treatment or prevention or alleviation of symptoms of certain gastrointestinal disorders. It is also hypothesized that the targeting of compounds to the gut may reduce the incidence of side effects due to systemic absorption of compounds.

In some embodiments, provided herein is a compound of Formula AA

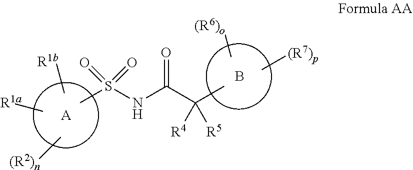

wherein
n=0 or 1;
o=1 or 2;
p=0, 1, 2, or 3;
wherein
A is a 5- to 10-membered heteroaryl or a C₆-C₁₀ aryl;
B is a 5- to 10-membered heteroaryl or a C₆-C₁₀ aryl;
wherein
R^{1a} is a C₁-C₆ alkyl, —CR¹¹R¹²NR¹¹R¹², or —SO₂NR¹¹R¹²;

wherein the C₁-C₆ alkyl is substituted with one or more hydroxy or —OSi(R¹³)₃;

R^{1b} is a C₁-C₆ alkyl substituted with one or more hydroxy, —SO₂NR¹¹R¹², —SO₂R¹³, —CONR¹¹R¹², —OR¹¹, —COR¹³; —CO₂R¹³, —NR¹³CONR¹¹R¹²; —CR¹¹R¹²CN, —NR¹¹SO₂R¹³, —NR¹¹CONR¹¹R¹², —CR¹¹R¹²NR¹¹R¹², CN, and —NR¹¹COR¹²;
at least one R⁶ is ortho to the bond connecting the B ring to the CR⁴R⁵ group of Formula AA;
R² is selected from C₁-C₆ alkyl, C₁-C₆ haloalkyl, C₁-C₆ alkoxy, C₁-C₆ haloalkoxy, halo, CN, NO₂, COC₁-C₆ alkyl, CO—C₆-C₁₀ aryl, CO(5- to 10-membered heteroaryl), CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, NHCOOC₁-C₆ alkyl, NH—(C═NR¹³)NR¹¹R¹², CONR⁸R⁹, SF₅, SC₁-C₆ alkyl, S(O₂)C₁-C₆ alkyl, S(O)C₁-C₆ alkyl, S(O₂)NR¹¹R¹², C₃-C₇ cycloalkyl and 3- to 7-membered heterocycloalkyl, wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), and OCO(3- to 7-membered heterocycloalkyl);
wherein each C₁-C₆ alkyl substituent and each C₁-C₆, alkoxy substituent of the R² C₃-C₇ cycloalkyl or of the R² 3- to 7-membered heterocycloalkyl is further optionally independently substituted with one to three hydroxy, halo, or oxo;
wherein the 3- to 7-membered heterocycloalkyl, C₆-C₁₀ aryl, 5- to 10-membered heteroaryl of the R² C₁-C₆ alkyl, the R² C₁-C₆ haloalkyl, the R² C₃-C₇ cycloalkyl, or the R² 3- to 7-membered heterocycloalkyl are optionally substituted with one or more substituents independently selected from halo, C₁-C₆ alkyl, and OC₁-C₆ alkyl;
   R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₃-C₆ alkyl, C₃-C₁₀ cycloalkyl and 3- to 10-membered heterocycloalkyl, and C₂-C₆ alkenyl,
   wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, halo, C₆-C₁₀ aryl or NR⁸R⁹, or wherein R⁶ or R⁷ is optionally fused to a five-to-seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;
   each of R⁴ and R⁵ is independently selected from hydrogen and C₁-C₆ alkyl;
   or R⁴ and R⁵, together with the carbon atom to which they are attached, form a C₃-C₈ cycloalkyl optionally independently substituted with one or more substituents independently selected from hydroxy, hydroxymethyl, halo, oxo, C₁-C₆ alkyl, C₁-C₆ alkoxy, and NR⁸R⁹;
   R¹⁰ is C₁-C₆ alkyl;
   each of R⁸ and R⁹ at each occurrence is independently selected from hydrogen, C₁-C₆ alkyl, (C═NR¹³)NR¹¹R¹², S(O)₂C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or
   R⁸ and R⁹ taken together with the nitrogen they are attached to form a 3- to 7-membered ring optionally containing one or more heteroatoms in addition to the nitrogen they are attached to;
   R¹³ is C₁-C₆ alkyl, C₆-C₁₀ aryl, or 5- to 10-membered heteroaryl;
   each of R¹¹ and R¹² at each occurrence is independently selected from hydrogen and C₁-C₆ alkyl optionally substituted with hydroxy, OR¹³, or O—(C₁-C₆ alkyl)-R¹³;
   with the proviso that the compound of Formula AA is not a compound selected from the group consisting of:

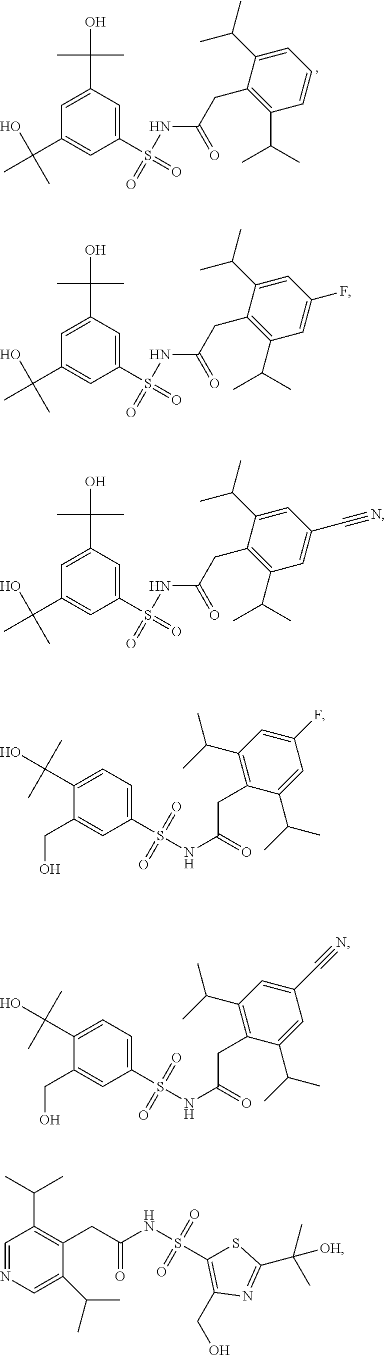 [SMILES: CC(C)c1cccc(C(C)C)c1CC(=O)NS(=O)(=O)c1cc(C(C)(C)O)cc(C(C)(C)O)c1 ; CC(C)c1cc(F)cc(C(C)C)c1CC(=O)NS(=O)(=O)c1cc(C(C)(C)O)cc(C(C)(C)O)c1 ; CC(C)c1cc(C#N)cc(C(C)C)c1CC(=O)NS(=O)(=O)c1cc(C(C)(C)O)cc(C(C)(C)O)c1 ; CC(C)c1cc(F)cc(C(C)C)c1CC(=O)NS(=O)(=O)c1ccc(C(C)(C)O)c(CO)c1 ; CC(C)c1cc(C#N)cc(C(C)C)c1CC(=O)NS(=O)(=O)c1ccc(C(C)(C)O)c(CO)c1 ; CC(C)c1cncc(C(C)C)c1CC(=O)NS(=O)(=O)c1sc(C(C)(C)O)nc1CO]

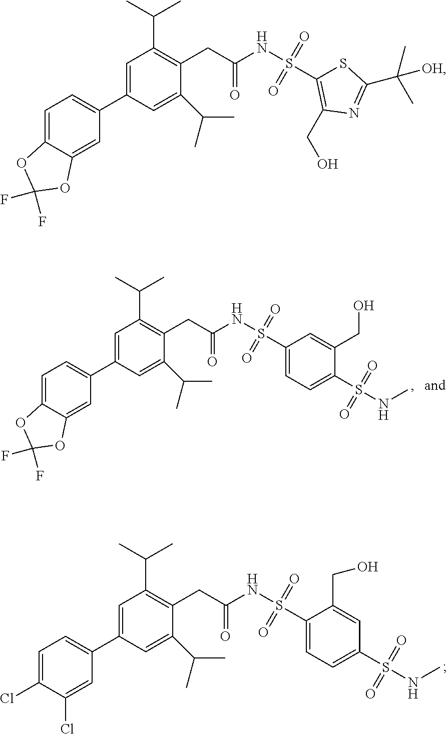 [SMILES: CC(C)c1cc(-c2ccc3c(c2)OC(F)(F)O3)cc(C(C)C)c1CC(=O)NS(=O)(=O)c1sc(C(C)(C)O)nc1CO ; CNS(=O)(=O)c1ccc(S(=O)(=O)NC(=O)Cc2c(C(C)C)cc(-c3ccc4c(c3)OC(F)(F)O4)cc2C(C)C)cc1CO ; CNS(=O)(=O)c1ccc(S(=O)(=O)NC(=O)Cc2c(C(C)C)cc(-c3ccc(Cl)c(Cl)c3)cc2C(C)C)c(CO)c1]

or a pharmaceutically acceptable salt thereof.

In some embodiments, provided herein is a compound of Formula AA

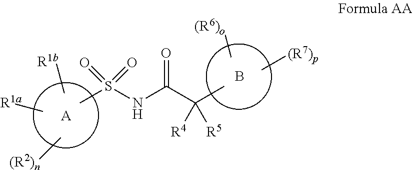

wherein
n=0 or 1;
o=1 or 2;
p=0, 1, 2, or 3;
wherein
A is a 5- to 10-membered heteroaryl or a C₆-C₁₀ aryl;
B is a 5- to 10-membered heteroaryl or a C₆-C₁₀ aryl;
wherein
R^{1a} is a C₁-C₆ alkyl, —CR¹¹R¹²NR¹¹R¹², or —SO₂NR¹¹R¹²;
wherein the C₁-C₆ alkyl is substituted with one or more hydroxy or —OSi(R¹³)₃;
   R^{1b} is a C₁-C₆ alkyl substituted with one or more hydroxy, —SO₂NR¹¹R¹², —SO₂R¹³, —CONR¹¹R¹², —OR¹¹, —COR¹³; —CO₂R¹³, —NR¹³CONR¹¹R¹²; —CR¹¹R¹²CN, —NR¹¹SO₂R¹³, —NR¹¹CONR¹¹R¹², —CR¹¹R¹²NR¹¹R¹², CN, and —NR¹¹COR¹²;
   at least one R⁶ is ortho to the bond connecting the B ring to the CR⁴R⁵ group of Formula AA;
   R² is selected from C₁-C₆ alkyl, C₁-C₆ haloalkyl, C₁-C₆ alkoxy, C₁-C₆ haloalkoxy, halo, CN, NO₂, COC₁-C₆ alkyl, CO—C₆-C₁₀ aryl, CO(5- to 10-membered heteroaryl), CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, NHCOOC₁-C₆ alkyl, NH—(C═NR¹³)NR¹¹R¹², CONR⁸R⁹, SF₅, SC₁-C₆ alkyl, S(O₂)C₁-C₆ alkyl, S(O)C₁-C₆ alkyl, S(O₂)NR¹¹R¹², C₃-C₇ cycloalkyl and 3- to 7-membered heterocycloalkyl, wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), and OCO(3- to 7-membered heterocycloalkyl);
wherein each C₁-C₆ alkyl substituent and each C₁-C₆ alkoxy substituent of the R² C₃-C₇ cycloalkyl or of the R² 3- to 7-membered heterocycloalkyl is further optionally independently substituted with one to three hydroxy, halo, or oxo;
wherein the 3- to 7-membered heterocycloalkyl, C₆-C₁₀ aryl, 5- to 10-membered heteroaryl of the R² C₁-C₆ alkyl, the R² C₁-C₆ haloalkyl, the R² C₃-C₇ cycloalkyl, or the R² 3- to 7-membered heterocycloalkyl are optionally substituted with one or more substituents independently selected from halo, C₁-C₆ alkyl, and OC₁-C₆ alkyl;
   R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₁₀ cycloalkyl and 3- to 10-membered heterocycloalkyl, and C₂-C₆ alkenyl,
   wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, halo, C₆-C₁₀ aryl or NR⁸R⁹, or wherein R⁶ or R⁷ is optionally fused to a five-to-seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;
   each of R⁴ and R⁵ is independently selected from hydrogen and C₁-C₆ alkyl;
   R¹⁰ is C₁-C₆ alkyl;
   each of R⁸ and R⁹ at each occurrence is independently selected from hydrogen, C₁-C₆ alkyl, (C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or
   R⁸ and R⁹ taken together with the nitrogen they are attached to form a 3- to 7-membered ring optionally containing one or more heteroatoms in addition to the nitrogen they are attached to;
   R¹³ is C₁-C₆ alkyl, C₆-C₁₀ aryl, or 5- to 10-membered heteroaryl;
   each of R¹¹ and R¹² at each occurrence is independently selected from hydrogen and C₁-C₆ alkyl optionally substituted with hydroxy;
   with the proviso that the compound of Formula AA is not a compound selected from the group consisting of:

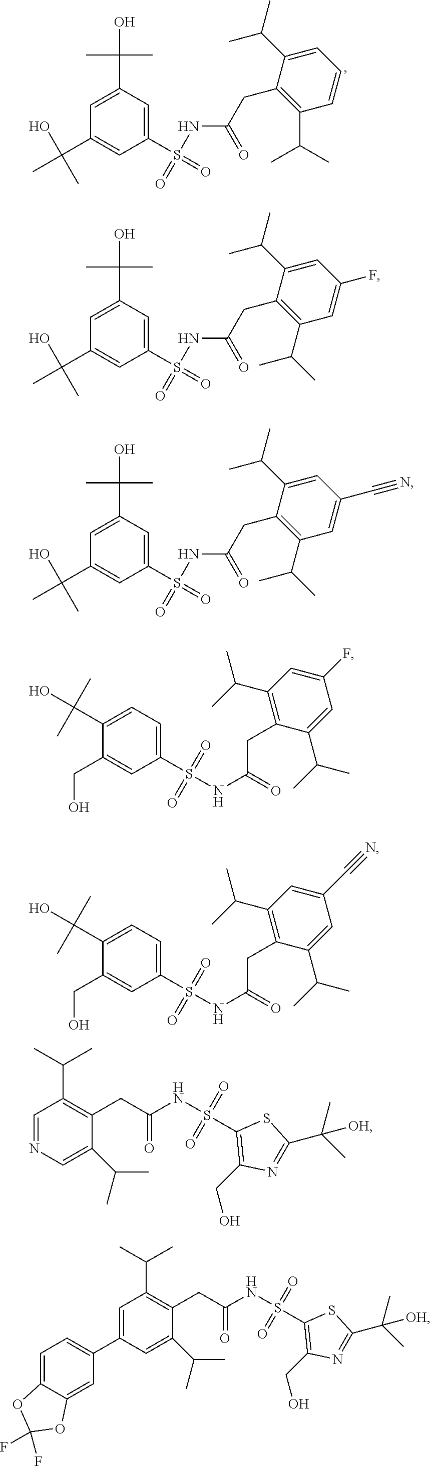

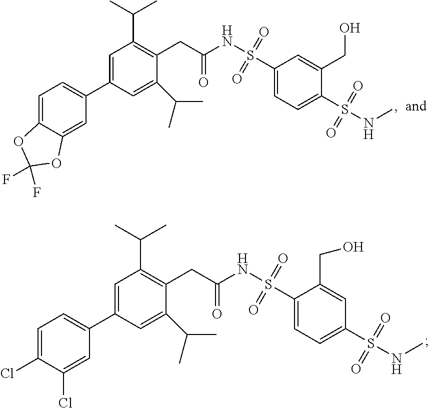 [SMILES: CNS(=O)(=O)c1ccc(S(=O)(=O)NC(=O)Cc2c(C(C)C)cc(-c3ccc4c(c3)OC(F)(F)O4)cc2C(C)C)cc1CO ; ",  and" ; CNS(=O)(=O)c1ccc(S(=O)(=O)NC(=O)Cc2c(C(C)C)cc(-c3ccc(Cl)c(Cl)c3)cc2C(C)C)c(CO)c1]

or a pharmaceutically acceptable salt thereof.

In some embodiments, provided herein is a compound of Formula AA

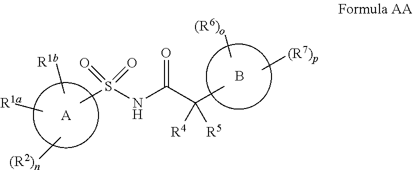

wherein
n=0 or 1;
o=1 or 2;
p=0, 1, 2, or 3;
wherein
A is a 5- to 10-membered heteroaryl or a C₆-C₁₀ aryl;
B is a 5- to 10-membered heteroaryl or a C₆-C₁₀ aryl;
wherein
R^{1a} is —SO₂NR¹¹R¹²;
R^{1b} is a C₁-C₆ alkyl substituted with one or more hydroxy, —SO₂NR¹¹R¹², —SO₂R¹³, —CONR¹¹R¹², —OR¹¹, —COR¹³; —CO₂R¹³, —NR¹³CONR¹¹R¹²; —CR¹¹R¹²CN, —NR¹¹SO₂R¹³, —NR¹¹CONR¹¹R¹², —CR¹¹R¹²NR¹¹R¹², CN, and —NR¹¹COR¹²;
at least one R⁶ is ortho to the bond connecting the B ring to the CR⁴R⁵ group of Formula AA;
R² is selected from C₁-C₆ alkyl, C₁-C₆ haloalkyl, C₁-C₆ alkoxy, C₁-C₆ haloalkoxy, halo, CN, NO₂, COC₁-C₆ alkyl, CO—C₆-C₁₀ aryl, CO(5- to 10-membered heteroaryl), CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, NHCOOC₁-C₆ alkyl, NH—(C═NR¹³)NR¹¹R¹², CONR⁸R⁹, SF₅, SC₁-C₆ alkyl, S(O₂)C₁-C₆ alkyl, S(O)C₁-C₆ alkyl, S(O₂)NR¹¹R¹², C₃-C₇ cycloalkyl and 3- to 7-membered heterocycloalkyl, wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), and OCO(3- to 7-membered heterocycloalkyl);
wherein each C₁-C₆ alkyl substituent and each C₁-C₆, alkoxy substituent of the R² C₃-C₇ cycloalkyl or of the R² 3- to 7-membered heterocycloalkyl is further optionally independently substituted with one to three hydroxy, halo, or oxo;
wherein the 3- to 7-membered heterocycloalkyl, C₆-C₁₀ aryl, 5- to 10-membered heteroaryl of the R² C₁-C₆ alkyl, the R² C₁-C₆ haloalkyl, the R² C₃-C₇ cycloalkyl, or the R² 3- to 7-membered heterocycloalkyl are optionally substituted with one or more substituents independently selected from halo, C₁-C₆ alkyl, and OC₁-C₆ alkyl;
   R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₃-C₆ alkyl, C₃-C₁₀ cycloalkyl and 3- to 10-membered heterocycloalkyl, and C₂-C₆ alkenyl,
   wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, halo, C₆-C₁₀ aryl or NR⁸R⁹, or wherein R⁶ or R⁷ is optionally fused to a five-to-seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;
   each of R⁴ and R⁵ is independently selected from hydrogen and C₁-C₆ alkyl;
   R¹⁰ is C₁-C₆ alkyl;
   each of R⁸ and R⁹ at each occurrence is independently selected from hydrogen, C₁-C₆ alkyl, (C═NR¹³)NR¹¹R¹², S(O)₂C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or
   R⁸ and R⁹ taken together with the nitrogen they are attached to form a 3- to 7-membered ring optionally containing one or more heteroatoms in addition to the nitrogen they are attached to;
   R¹³ is C₁-C₆ alkyl, C₆-C₁₀ aryl, or 5- to 10-membered heteroaryl;
   each of R¹¹ and R¹² at each occurrence is independently selected from hydrogen and C₁-C₆ alkyl optionally substituted with hydroxy;
   with the proviso that the compound of Formula AA is not a compound selected from the group consisting of:

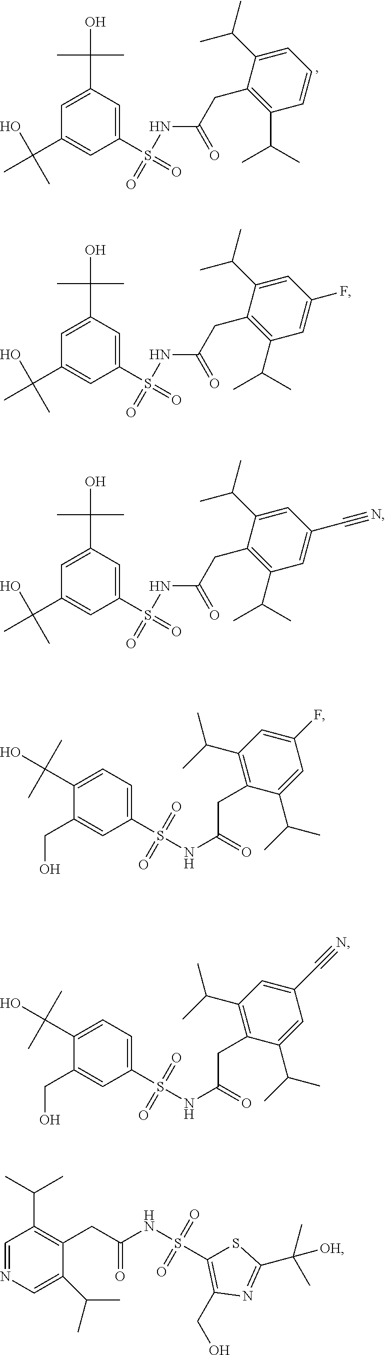 [SMILES: CC(C)c1cccc(C(C)C)c1CC(=O)NS(=O)(=O)c1cc(C(C)(C)O)cc(C(C)(C)O)c1 ; CC(C)c1cc(F)cc(C(C)C)c1CC(=O)NS(=O)(=O)c1cc(C(C)(C)O)cc(C(C)(C)O)c1 ; CC(C)c1cc(C#N)cc(C(C)C)c1CC(=O)NS(=O)(=O)c1cc(C(C)(C)O)cc(C(C)(C)O)c1 ; CC(C)c1cc(F)cc(C(C)C)c1CC(=O)NS(=O)(=O)c1ccc(C(C)(C)O)c(CO)c1 ; CC(C)c1cc(C#N)cc(C(C)C)c1CC(=O)NS(=O)(=O)c1ccc(C(C)(C)O)c(CO)c1 ; CC(C)c1cncc(C(C)C)c1CC(=O)NS(=O)(=O)c1sc(C(C)(C)O)nc1CO]

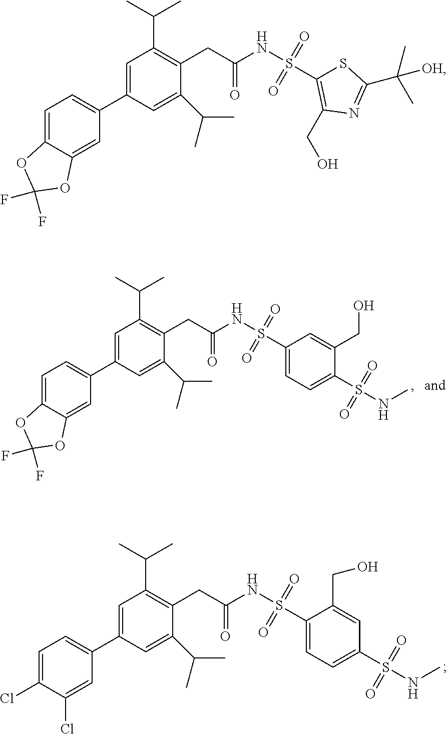 [SMILES: CC(C)c1cc(-c2ccc3c(c2)OC(F)(F)O3)cc(C(C)C)c1CC(=O)NS(=O)(=O)c1sc(C(C)(C)O)nc1CO ; CNS(=O)(=O)c1ccc(S(=O)(=O)NC(=O)Cc2c(C(C)C)cc(-c3ccc4c(c3)OC(F)(F)O4)cc2C(C)C)cc1CO ; CNS(=O)(=O)c1ccc(S(=O)(=O)NC(=O)Cc2c(C(C)C)cc(-c3ccc(Cl)c(Cl)c3)cc2C(C)C)c(CO)c1]

or a pharmaceutically acceptable salt thereof.

In some embodiments, provided herein is a compound of Formula AA

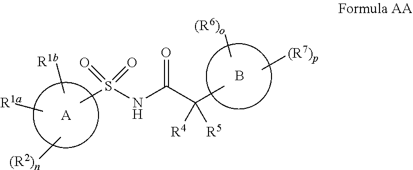

wherein the compound of Formula AA is selected from

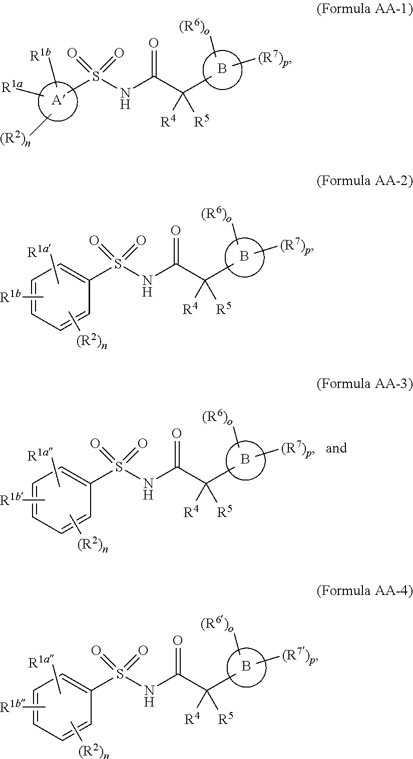

wherein
n=0 or 1;
o=1 or 2;
p=0, 1, 2, or 3;
wherein
A′ is a 5- to 10-membered heteroaryl;
B is a 5- to 10-membered heteroaryl or a C₆-C₁₀ aryl;
wherein
R^{1a} is a C₁-C₆ alkyl, —CR¹¹R¹²NR¹¹R¹² or —SO₂NR¹¹R¹²;
wherein the C₁-C₆ alkyl is substituted with one or more hydroxy or —OSi(R¹³)₃;
   R^{1a′} is a C₁-C₆ alkyl, —CR¹¹R¹²NR¹¹R¹² or —SO₂NR¹¹R¹²;
wherein the C₁-C₆ alkyl is substituted with one or more —OSi(R¹³)₃;
   R^{1a″} is a C₁-C₆ alkyl;
wherein the C₁-C₆ alkyl is substituted with one or more hydroxy;
   R^{1b} is a C₁-C₆ alkyl substituted with one or more hydroxy, —SO₂NR¹¹R¹², —SO₂R¹³, —CONR¹¹R¹², —OR¹¹, —COR¹³; —CO₂R¹³, —NR¹³CONR¹¹R¹²; —CR¹¹R¹²CN, —NR¹¹SO₂R¹³, —NR¹¹CONR¹¹R¹², —CR¹¹R¹²NR¹¹R¹², CN, and —NR¹¹COR¹²;
   R^{1b′} is —SO₂NR¹¹R¹², —SO₂R¹³, —CONR¹¹R¹², —OR¹¹, —COR¹³; —CO₂R¹³, —NR¹³CONR¹¹R¹²; —CR¹¹R¹²CN, —NR¹¹SO₂R¹³, —NR¹¹CONR¹¹R¹², —CR¹¹R¹²NR¹¹R¹², —CN, and —NR¹¹COR¹²;
   R^{1b″} is a C₁-C₆ alkyl;
wherein the C₁-C₆ alkyl is substituted with one or more hydroxy;
   at least one R⁶ is ortho to the bond connecting the B ring to the CR⁴R⁵ group of Formula AA-through Formula AA-1, AA-2, and AA-3;
   at least one R⁶ is ortho to the bond connecting the B ring to the CR⁴R⁵ group of Formula AA-4;
   R² is selected from C₁-C₆ alkyl, C₁-C₆ haloalkyl, C₁-C₆ alkoxy, C₁-C₆ haloalkoxy, halo, CN, NO₂, COC₁-C₆ alkyl, CO—C₆-C₁₀ aryl, CO(5- to 10-membered heteroaryl), CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, NHCOOC₁-C₆ alkyl, NH—(C═NR¹³)NR¹¹R¹², CONR⁸R⁹, SF₅, SC₁-C₆ alkyl, S(O₂)C₁-C₆ alkyl, S(O)C₁-C₆ alkyl, S(O₂)NR¹¹R¹², C₃-C₇ cycloalkyl, and 3- to 7-membered heterocycloalkyl, wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), and OCO(3- to 7-membered heterocycloalkyl);
wherein each C₁-C₆ alkyl substituent and each C₁-C₆, alkoxy substituent of the R² C₃-C₇ cycloalkyl or of the R² 3- to 7-membered heterocycloalkyl is further optionally independently substituted with one to three hydroxy, halo, or oxo;
wherein the 3- to 7-membered heterocycloalkyl, C₆-C₁₀ aryl, 5- to 10-membered heteroaryl of the R² C₁-C₆ alkyl, the R² C₁-C₆ haloalkyl, the R² C₃-C₇ cycloalkyl, or the R² 3- to 7-membered heterocycloalkyl are optionally substituted with one or more substituents independently selected from halo, C₁-C₆ alkyl, and OC₁-C₆ alkyl;
   R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₁₀ cycloalkyl and 3- to 10-membered heterocycloalkyl, and C₂-C₆ alkenyl,
   wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, O(C₃-C₁₀ cycloalkyl), and S(O₂)C₁-C₆ alkyl; and wherein the C₁-C₆ alkyl or C₁-C₆ alkoxy that R⁶ or R⁷ is substituted with is optionally substituted with one or more hydroxyl, halo, C₆-C₁₀ aryl or NR⁸R⁹, or wherein R⁶ or R⁷ is optionally fused to a five-to-seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;
   R^{6′} and R^{7′} are each independently selected from C₁-C₆ alkyl, C₁-C₆ haloalkyl, C₁-C₆ alkoxy, C₁-C₆ haloalkoxy, Cl, Br, I, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₁₀ cycloalkyl and 3- to 10-membered heterocycloalkyl, and C₂-C₆ alkenyl,
   wherein R^{6′} and R^{7′} are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, O(C₃-C₁₀ cycloalkyl), and S(O₂)C₁-C₆ alkyl; and wherein the C₁-C₆ alkyl or C₁-C₆ alkoxy that R⁶′ or R^{7′} is substituted with is optionally substituted with one or more hydroxyl, halo, C₆-C₁₀ aryl or NR⁸R⁹, or wherein R⁶ or R⁷ is optionally fused to a five-to-seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or at least one pair of R^{6′} and R^{7′} on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;
   each of R⁴ and R⁵ is independently selected from hydrogen and C₁-C₆ alkyl;
   or R⁴ and R⁵, together with the carbon atom to which they are attached, form a C₃-C₈ cycloalkyl optionally independently substituted with one or more substituents independently selected from hydroxy, hydroxymethyl, halo, oxo, C₁-C₆ alkyl, C₁-C₆ alkoxy, and NR⁸R⁹;
   R¹⁰ is C₁-C₆ alkyl;
   each of R⁸ and R⁹ at each occurrence is independently selected from hydrogen, C₁-C₆ alkyl, (C═NR¹³)NR¹¹R¹², S(O)₂C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or
   R⁸ and R⁹ taken together with the nitrogen they are attached to form a 3- to 7-membered ring optionally containing one or more heteroatoms in addition to the nitrogen they are attached to;
   R¹³ is C₁-C₆ alkyl, C₆-C₁₀ aryl, or 5- to 10-membered heteroaryl;
   each of R¹¹ and R¹² at each occurrence is independently selected from hydrogen and C₁-C₆ alkyl optionally substituted with hydroxy, OR¹³, or O—(C₁-C₆ alkyl)-R¹³;
   with the proviso that the compound of Formula AA is not a compound selected from the group consisting of:

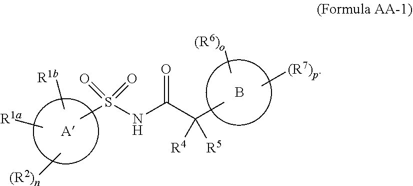

or a pharmaceutically acceptable salt thereof.

In some embodiments the variables shown in the formulae herein are as follows:

### The Formula AA

In some embodiments, Formula AA is Formula AA-1

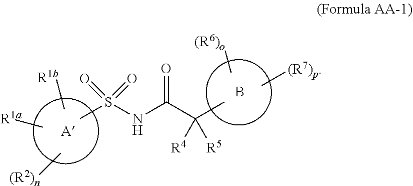

In some embodiments, Formula AA is Formula AA-2

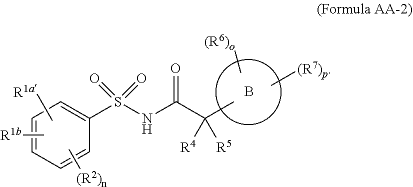

In some embodiments, Formula AA is Formula AA-3

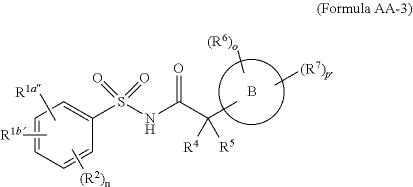

In some embodiments, Formula AA is Formula AA-4

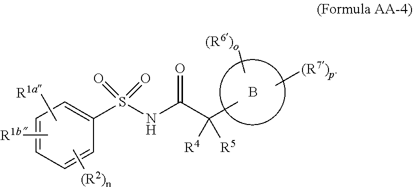

### The Variable n

In some embodiments n=0 or 1. In some embodiments n=0. In some embodiments n=1.

### The Ring a and Substitutions on the Ring A

In some embodiments, A is a 5- to 10-membered heteroaryl. In some embodiments, A is a 5- to 6-membered heteroaryl. In some embodiments, A is 5-membered heteroaryl. In some embodiments, A is 6-membered heteroaryl. In some embodiments, A is 10-membered heteroaryl. In some embodiments, A is a monocyclic heteroaryl. In some embodiments, A is a bicyclic heteroaryl. In some embodiments, A is 5-membered heteroaryl including 1-2 (e.g., 1) nitrogen ring members. In some embodiments, A is 5-membered heteroaryl including 1 nitrogen ring member and 1 oxygen ring member. In some embodiments, A is oxazolyl, and n is 0. In some embodiments, A is isoxazolyl, and n is 0. In some embodiments, A is pyrazolyl, and n is 0. In some embodiments, A is pyrazolyl, and n is 1. In some embodiments, A is imidazolyl, and n is 0. In some embodiments, A is imidazolyl, and n is 1. In some embodiments, A is thiazolyl, and n is 0. In some embodiments, A is a 5- to 6-membered (e.g., 5-membered) heteroaryl containing 1-2 sulfur ring members. In some embodiments, A is a 5-membered heteroaryl containing 1 sulfur ring member. In some embodiments, A is a 5-membered heteroaryl containing a sulfur ring member and one or more nitrogen ring member. In some embodiments, A is a 5-membered heteroaryl containing a sulfur ring member and a nitrogen ring member. In some embodiments, A is thiophenyl, and n is 0. In some embodiments, A is thiophenyl, and n is 1. In some embodiments, A is thiazolyl, and n is 0. In some embodiments, A is isothiazolyl, and n is 0.

In some embodiments, A is C₆-C₁₀ aryl. In some embodiments, A is phenyl. In some embodiments, A is phenyl, and n is 0.

### The Ring A′ and Substitutions on the Ring A′

In some embodiments, A′ is a 5- to 10-membered heteroaryl. In some embodiments, A′ is a 5- to 6-membered heteroaryl. In some embodiments, A′ is 5-membered heteroaryl. In some embodiments, A′ is 6-membered heteroaryl. In some embodiments, A′ is 10-membered heteroaryl. In some embodiments, A′ is a monocyclic heteroaryl. In some embodiments, A′ is a bicyclic heteroaryl. In some embodiments, A′ is 5-membered heteroaryl including 1-2 (e.g., 1) nitrogen ring members. In some embodiments, A′ is 5-membered heteroaryl including 1 nitrogen ring member and 1 oxygen ring member. In some embodiments, A′ is oxazolyl, and n is 0. In some embodiments, A′ is isoxazolyl, and n is 0. In some embodiments, A′ is imidazolyl, and n is 0. In some embodiments, A′ is imidazolyl, and n is 1. In some embodiments, A′ is thiazolyl, and n is 0. In some embodiments, A′ is a 5- to 6-membered (e.g., 5-membered) heteroaryl containing 1-2 sulfur ring members. In some embodiments, A′ is a 5-membered heteroaryl containing 1 sulfur ring member. In some embodiments, A′ is a 5-membered heteroaryl containing a sulfur ring member and one or more nitrogen ring member. In some embodiments, A′ is a 5-membered heteroaryl containing a sulfur ring member and a nitrogen ring member.

In some embodiments, A′ is thiophenyl, and n is 0. In some embodiments, A′ is thiophenyl, and n is 1. In some embodiments, A′ is thiazolyl, and n is 0. In some embodiments, A′ is isothiazolyl, and n is 0.

In some embodiments, the substituted ring A

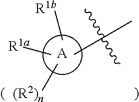

In some embodiments, the substituted ring A is

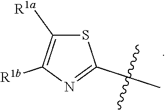

In some embodiments, the substituted ring A is

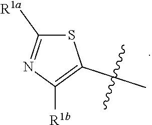

In some embodiments, the substituted ring A is

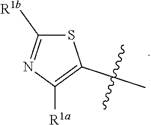

In some embodiments, the substituted ring A is

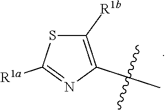

In some embodiments, the substituted ring A is

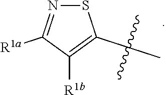

In some embodiments, the substituted ring A is

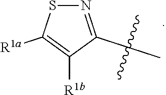

In some embodiments, the substituted ring A is

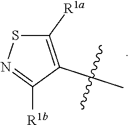

In some embodiments, the substituted ring A is

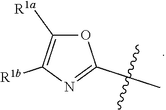

In some embodiments, the substituted ring A is

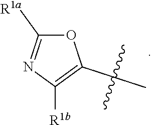

In some embodiments, the substituted ring A is

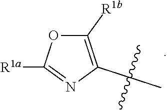

In some embodiments, the substituted ring A is

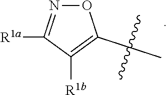

In some embodiments, the substituted ring A is

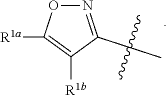

In some embodiments, the substituted ring A is

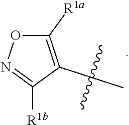

In some embodiments, the substituted ring A is

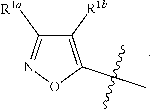

In some embodiments, the substituted ring A is

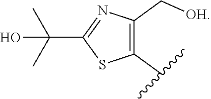 [SMILES: *c1sc(C(C)(C)O)nc1CO]

In some embodiments, the substituted ring A is

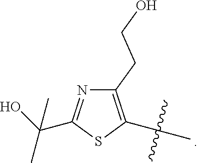 [SMILES: *c1sc(C(C)(C)O)nc1CCO]

In some embodiments, A is C₆-C₁₀ aryl. In some embodiments, A is

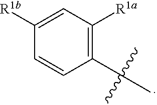

In some embodiments, A is

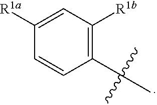

In some embodiments, A is

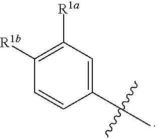

In some embodiments, A is

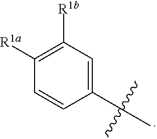

In some embodiments, A is

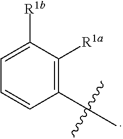

In some embodiments, A is

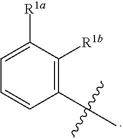

In some embodiments, A is

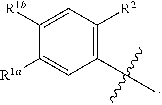

In some embodiments, A is

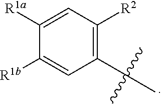

In some embodiments, A is

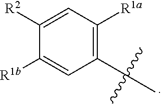

In some embodiments, A is

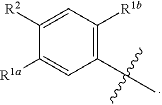

In some embodiments, A is

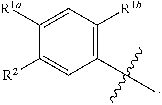

In some embodiments, A is

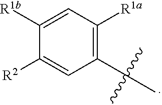

In some embodiments, A is

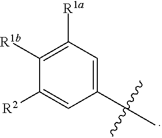

In some embodiments, A is

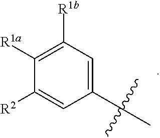

In some embodiments, A is

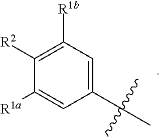

In some embodiments, A is

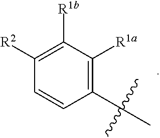

In some embodiments, A is

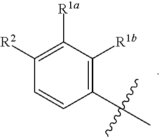

In some embodiments, A is

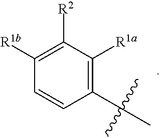

In some embodiments, A is

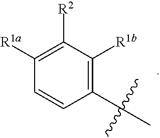

In some embodiments, A is

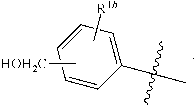

In some embodiments, A is

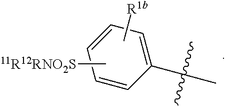

In some embodiments, A is

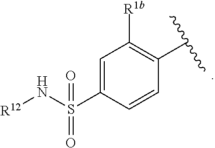

In some embodiments, A is

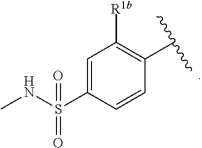

In some embodiments, A is

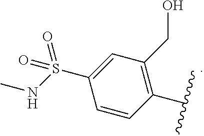 [SMILES: *c1ccc(S(=O)(=O)NC)cc1CO]

In some embodiments, A is

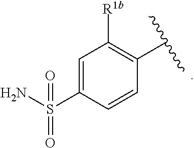

In some embodiments, A is

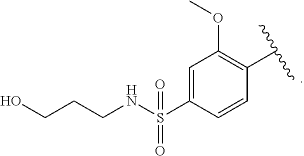 [SMILES: *c1ccc(S(=O)(=O)NCCCO)cc1OC]

In some embodiments, A is

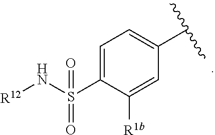

In some embodiments, A is

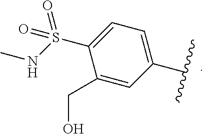 [SMILES: *c1ccc(S(=O)(=O)NC)c(CO)c1]

In some embodiments, A is

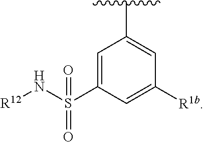

In some embodiments, A is

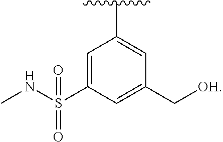 [SMILES: *c1cc(CO)cc(S(=O)(=O)NC)c1]

In some embodiments, A is

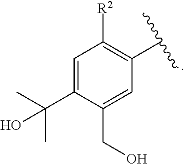

In some embodiments, A is

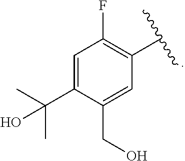 [SMILES: *c1cc(CO)c(C(C)(C)O)cc1F]

In some embodiments, A is

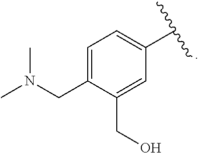 [SMILES: *c1ccc(CN(C)C)c(CO)c1]

In some embodiments, the substituted ring A′ is

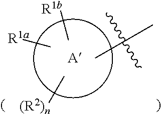

In some embodiments, the substituted ring A′ is

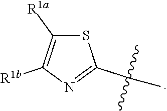

In some embodiments, the substituted ring A′ is

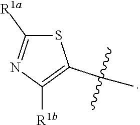

In some embodiments, the substituted ring A′ is

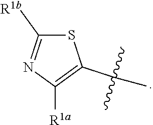

In some embodiments, the substituted ring A′ is

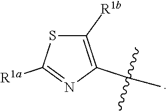

In some embodiments, the substituted ring A′ is

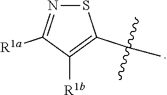

In some embodiments, the substituted ring A′ is

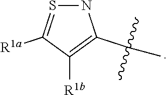

In some embodiments, the substituted ring A′ is

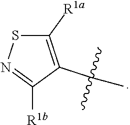

In some embodiments, the substituted ring A′ is

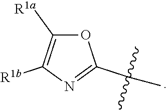

In some embodiments, the substituted ring A′ is

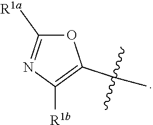

In some embodiments, the substituted ring A′ is

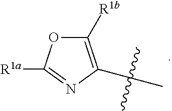

In some embodiments, the substituted ring A′ is

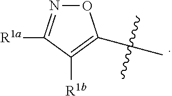

In some embodiments, the substituted ring A′ is

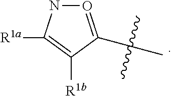

In some embodiments, the substituted ring A′ is

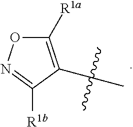

In some embodiments, the substituted ring A′ is

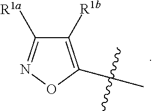

In some embodiments, the substituted ring A′ is

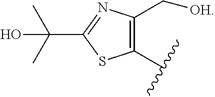 [SMILES: *c1sc(C(C)(C)O)nc1CO]

In some embodiments, the substituted ring A′ is

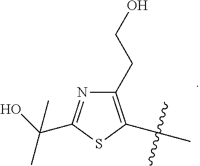 [SMILES: *c1sc(C(C)(C)O)nc1CCO]

The groups R^{1a}, R^{1a′}, R^{1a″}, R^{1b}, R^{1b′}, and R^{1b″}

In some embodiments, R^{1a} is C₁-C₆ alkyl substituted with one or more hydroxy. In some embodiments, R^{1a} is C₁-C₆ alkyl substituted with one or more —OSi(R¹³)₃. In some embodiments, R^{1a} is —CR¹¹R¹²NR¹¹R¹². In some embodiments, R^{1a} is —SO₂NR¹¹R¹².

In some embodiments, R^{1a} is C₁-C₆ alkyl substituted with one or more —OSi(R¹³)₃. In some embodiments, R^{1a} is —CR¹¹R¹²NR¹¹R¹². In some embodiments, R^{1a} is —SO₂NR¹¹R¹².

In some embodiments, R^{1b} is a C₁-C₆ alkyl substituted with one or more hydroxyl. In some embodiments, R^{1b} is —SO₂NR¹¹R¹². In some embodiments, R^{1b} is —SO₂R¹³. In some embodiments, R^{1b} is —CONR¹¹R¹². In some embodiments, R^{1b} is —OR¹¹. In some embodiments, R^{1b} is —COR¹³. In some embodiments, R^{1b} is —CO₂R¹³. In some embodiments, R^{1b} is —NR¹³CONR¹¹R¹¹. In some embodiments, R^{1b} is —CR¹¹R¹²CN. In some embodiments, R^{1b} is —NR¹¹SO₂R¹³. In some embodiments, R^{1b} is —NR¹¹CONR¹¹R¹². In some embodiments, R^{1b} is —CR¹¹R¹²NR¹¹R¹². In some embodiments, R^{1b} is —CN. In some embodiments, R^{1b} is —NR¹¹COR¹².

In some embodiments, one of R^{1a} and R^{1b} is C₁-C₆ alkyl substituted by one hydroxy, and the other one of R^{1a} and R^{1b} is C₁-C₆ alkyl substituted by one hydroxy. In some embodiments, one of R^{1a} and R^{1b} is C₁-C₆ alkyl substituted by two hydroxy, and the other one of R^{1a} and R^{1b} is C₁-C₆ alkyl substituted by one hydroxy. In some embodiments, one of R^{1a} and R^{1b} is C₁-C₅ alkyl substituted by one hydroxy, and the other one of R^{1a} and R^{1b} is C₁-C₅ alkyl substituted by one hydroxy. In some embodiments, one of R^{1a} and R^{1b} is C₁ alkyl substituted by one hydroxy, and the other one of R^{1a} and R^{1b} is C₁ alkyl substituted by one hydroxy. In some embodiments, one of R^{1a} and R^{1b} is C₁ alkyl substituted by one hydroxy, and the other one of R^{1a} and R^{1b} is C₂ alkyl substituted by one hydroxy. In some embodiments, one of R^{1a} and R^{1b} is C₁ alkyl substituted by one hydroxy, and the other one of R^{1a} and R^{1b} is C₃ alkyl substituted by one hydroxy. In some embodiments, one of R^{1a} and R^{1b} is C₁ alkyl substituted by one hydroxy, and the other one of R^{1a} and R^{1b} is C₄ alkyl substituted by one hydroxy. In some embodiments, one of R^{1a} and R^{1b} is C₁ alkyl substituted by one hydroxy, and the other one of R^{1a} and R^{1b} is C₅ alkyl substituted by one hydroxy. In some embodiments, one of R^{1a} and R^{1b} is C₁ alkyl substituted by one hydroxy, and the other one of R^{1a} and R^{1b} is C₆ alkyl substituted by one hydroxy. In some embodiments, one of R^{1a} and R^{1b} is C₂ alkyl substituted by one hydroxy, and the other one of R^{1a} and R^{1b} is C₁ alkyl substituted by one hydroxy. In some embodiments, one of R^{1a} and R^{1b} is C₂ alkyl substituted by one hydroxy, and the other one of R^{1a} and R^{1b} is C₂ alkyl substituted by one hydroxy. In some embodiments, one of R^{1a} and R^{1b} is C₂ alkyl substituted by one hydroxy, and the other one of R^{1a} and R^{1b} is C₃ alkyl substituted by one hydroxy. In some embodiments, one of R^{1a} and R^{1b} is C₂ alkyl substituted by one hydroxy, and the other one of R^{1a} and R^{1b} is C₄ alkyl substituted by one hydroxy. In some embodiments, one of R^{1a} and R^{1b} is C₂ alkyl substituted by one hydroxy, and the other one of R^{1a} and R^{1b} is C₅ alkyl substituted by one hydroxy. In some embodiments, one of R^{1a} and R^{1b} is C₂ alkyl substituted by one hydroxy, and the other one of R^{1a} and R^{1b} is C₆ alkyl substituted by one hydroxy. In some embodiments, one of R^{1a} and R^{1b} is C₃ alkyl substituted by one hydroxy, and the other one of R^{1a} and R^{1b} is C₁ alkyl substituted by one hydroxy. In some embodiments, one of R^{1a} and R^{1b} is C₃ alkyl substituted by one hydroxy, and the other one of R^{1a} and R^{1b} is C₂ alkyl substituted by one hydroxy. In some embodiments, one of R^{1a} and R^{1b} is C₃ alkyl substituted by one hydroxy, and the other one of R^{1a} and R^{1b} is C₃ alkyl substituted by one hydroxy. In some embodiments, one of R^{1a} and R^{1b} is C₃ alkyl substituted by one hydroxy, and the other one of R^{1a} and R^{1b} is C₄ alkyl substituted by one hydroxy. In some embodiments, one of R^{1a} and R^{1b} is C₃ alkyl substituted by one hydroxy, and the other one of R^{1a} and R^{1b} is C₅ alkyl substituted by one hydroxy. In some embodiments, one of R^{1a} and R^{1b} is C₃ alkyl substituted by one hydroxy, and the other one of R^{1a} and R^{1b} is C₆ alkyl substituted by one hydroxy. In some embodiments, one of R^{1a} and R^{1b} is C₄ alkyl substituted by one hydroxy, and the other one of R^{1a} and R^{1b} is C₁ alkyl substituted by one hydroxy. In some embodiments, one of R^{1a} and R^{1b} is C₄ alkyl substituted by one hydroxy, and the other one of R^{1a} and R^{1b} is C₂ alkyl substituted by one hydroxy. In some embodiments, one of R^{1a} and R^{1b} is C₄ alkyl substituted by one hydroxy, and the other one of R^{1a} and R^{1b} is C₃ alkyl substituted by one hydroxy. In some embodiments, one of R^{1a} and R^{1b} is C₄ alkyl substituted by one hydroxy, and the other one of R^{1a} and R^{1b} is C₄ alkyl substituted by one hydroxy. In some embodiments, one of R^{1a} and R^{1b} is C₄ alkyl substituted by one hydroxy, and the other one of R^{1a} and R^{1b} is C₅ alkyl substituted by one hydroxy. In some embodiments, one of R^{1a} and R^{1b} is C₄ alkyl substituted by one hydroxy, and the other one of R^{1a} and R^{1b} is C₆ alkyl substituted by one hydroxy. In some embodiments, one of R^{1a} and R^{1b} is C₅ alkyl substituted by one hydroxy, and the other one of R^{1a} and R^{1b} is C₁ alkyl substituted by one hydroxy. In some embodiments, one of R^{1a} and R^{1b} is C₅ alkyl substituted by one hydroxy, and the other one of R^{1a} and R^{1b} is C₂ alkyl substituted by one hydroxy. In some embodiments, one of R^{1a} and R^{1b} is C₅ alkyl substituted by one hydroxy, and the other one of R^{1a} and R^{1b} is C₃ alkyl substituted by one hydroxy. In some embodiments, one of R^{1a} and R^{1b} is C₅ alkyl substituted by one hydroxy, and the other one of R^{1a} and R^{1b} is C₄ alkyl substituted by one hydroxy. In some embodiments, one of R^{1a} and R^{1b} is C₅ alkyl substituted by one hydroxy, and the other one of R^{1a} and R^{1b} is C₅ alkyl substituted by one hydroxy. In some embodiments, one of R^{1a} and R^{1b} is C₅ alkyl substituted by one hydroxy, and the other one of R^{1a} and R^{1b} is C₆ alkyl substituted by one hydroxy. One of R^{1a} and R^{1b} is C₆ alkyl substituted by one hydroxy, and the other one of R^{1a} and R^{1b} is C₁ alkyl substituted by one hydroxy. In some embodiments, one of R^{1a} and R^{1b} is C₆ alkyl substituted by one hydroxy, and the other one of R^{1a} and R^{1b} is C₂ alkyl substituted by one hydroxy. In some embodiments, one of R^{1a} and R^{1b} is C₆ alkyl substituted by one hydroxy, and the other one of R^{1a} and R^{1b} is C₃ alkyl substituted by one hydroxy. In some embodiments, one of R^{1a} and R^{1b} is C₆ alkyl substituted by one hydroxy, and the other one of R^{1a} and R^{1b} is C₄ alkyl substituted by one hydroxy. In some embodiments, one of R^{1a} and R^{1b} is C₆ alkyl substituted by one hydroxy, and the other one of R^{1a} and R^{1b} is C₅ alkyl substituted by one hydroxy. In some embodiments, one of R^{1a} and R^{1b} is C₆ alkyl substituted by one hydroxy, and the other one of R^{1a} and R^{1b} is C₆ alkyl substituted by one hydroxy. In some embodiments, one of R^{1a} and R^{1b} is C₁ alkyl substituted by one hydroxy, and the other one of R^{1a} and R^{1b} is C₂ alkyl substituted by two hydroxy. In some embodiments, one of R^{1a} and R^{1b} is C₁ alkyl substituted by one hydroxy, and the other one of R^{1a} and R^{1b} is C₃ alkyl substituted by two hydroxy. In some embodiments, one of R^{1a} and R^{1b} is C₁ alkyl substituted by one hydroxy, and the other one of R^{1a} and R^{1b} is C₄ alkyl substituted by two hydroxy. In some embodiments, one of R^{1a} and R^{1b} is C₁ alkyl substituted by one hydroxy, and the other one of R^{1a} and R^{1b} is C₅ alkyl substituted by two hydroxy. In some embodiments, one of R^{1a} and R^{1b} is C₁ alkyl substituted by one hydroxy, and the other one of R^{1a} and R^{1b} is C₆ alkyl substituted by two hydroxy. In some embodiments, one of R^{1a} and R^{1b} is C₂ alkyl substituted by one hydroxy, and the other one of R^{1a} and R^{1b} is C₂ alkyl substituted by two hydroxy. In some embodiments, one of R^{1a} and R^{1b} is C₂ alkyl substituted by one hydroxy, and the other one of R^{1a} and R^{1b} is C₃ alkyl substituted by two hydroxy. In some embodiments, one of R^{1a} and R^{1b} is C₂ alkyl substituted by one hydroxy, and the other one of R^{1a} and R^{1b} is C₄ alkyl substituted by two hydroxy. In some embodiments, one of R^{1a} and R^{1b} is C₂ alkyl substituted by one hydroxy, and the other one of R^{1a} and R^{1b} is C₅ alkyl substituted by two hydroxy. In some embodiments, one of R^{1a} and R^{1b} is C₂ alkyl substituted by one hydroxy, and the other one of R^{1a} and R^{1b} is C₆ alkyl substituted by two hydroxy. In some embodiments, one of R^{1a} and R^{1b} is C₃ alkyl substituted by one hydroxy, and the other one of R^{1a} and R^{1b} is C₂ alkyl substituted by two hydroxy. In some embodiments, one of R^{1a} and R^{1b} is C₃ alkyl substituted by one hydroxy, and the other one of R^{1a} and R^{1b} is C₃ alkyl substituted by two hydroxy. In some embodiments, one of R^{1a} and R^{1b} is C₃ alkyl substituted by one hydroxy, and the other one of R^{1a} and R^{1b} is C₄ alkyl substituted by two hydroxy. In some embodiments, one of R^{1a} and R^{1b} is C₃ alkyl substituted by one hydroxy, and the other one of R^{1a} and R^{1b} is C₅ alkyl substituted by two hydroxy. In some embodiments, one of R^{1a} and R^{1b} is C₃ alkyl substituted by one hydroxy, and the other one of R^{1a} and R^{1b} is C₆ alkyl substituted by two hydroxy. In some embodiments, one of R^{1a} and R^{1b} is C₄ alkyl substituted by one hydroxy, and the other one of R^{1a} and R^{1b} is C₂ alkyl substituted by two hydroxy. In some embodiments, one of R^{1a} and R^{1b} is C₄ alkyl substituted by one hydroxy, and the other one of R^{1a} and R^{1b} is C₃ alkyl substituted by two hydroxy. In some embodiments, one of R^{1a} and R^{1b} is C₄ alkyl substituted by one hydroxy, and the other one of R^{1a} and R^{1b} is C₄ alkyl substituted by two hydroxy. In some embodiments, one of R^{1a} and R^{1b} is C₄ alkyl substituted by one hydroxy, and the other one of R^{1a} and R^{1b} is C₅ alkyl substituted by two hydroxy. In some embodiments, one of R^{1a} and R^{1b} is C₄ alkyl substituted by one hydroxy, and the other one of R^{1a} and R^{1b} is C₆ alkyl substituted by two hydroxy. In some embodiments, one of R^{1a} and R^{1b} is C₅ alkyl substituted by one hydroxy, and the other one of R^{1a} and R^{1b} is C₂ alkyl substituted by two hydroxy. In some embodiments, one of R^{1a} and R^{1b} is C₅ alkyl substituted by one hydroxy, and the other one of R^{1a} and R^{1b} is C₃ alkyl substituted by two hydroxy. In some embodiments, one of R^{1a} and R^{1b} is C₅ alkyl substituted by one hydroxy, and the other one of R^{1a} and R^{1b} is C₄ alkyl substituted by two hydroxy. In some embodiments, one of R^{1a} and R^{1b} is C₅ alkyl substituted by one hydroxy, and the other one of R^{1a} and R^{1b} is C₅ alkyl substituted by two hydroxy. In some embodiments, one of R^{1a} and R^{1b} is C₅ alkyl substituted by one hydroxy, and the other one of R^{1a} and R^{1b} is C₆ alkyl substituted by two hydroxy. In some embodiments, one of R^{1a} and R^{1b} is C₆ alkyl substituted by one hydroxy, and the other one of R^{1a} and R^{1b} is C₂ alkyl substituted by two hydroxy. In some embodiments, one of R^{1a} and R^{1b} is C₆ alkyl substituted by one hydroxy, and the other one of R^{1a} and R^{1b} is C₃ alkyl substituted by two hydroxy. In some embodiments, one of R^{1a} and R^{1b} is C₆ alkyl substituted by one hydroxy, and the other one of R^{1a} and R^{1b} is C₄ alkyl substituted by two hydroxy. In some embodiments, one of R^{1a} and R^{1b} is C₆ alkyl substituted by one hydroxy, and the other one of R^{1a} and R^{1b} is C₅ alkyl substituted by two one hydroxy. In some embodiments, one of R^{1a} and R^{1b} is C₆ alkyl substituted by one hydroxy, and the other one of R^{1a} and R^{1b} is C₆ alkyl substituted by two hydroxy.

In some embodiments of any of the formulae herein, hydroxyethyl is 1-hydroxyethyl. In some embodiments of any of the formulae herein, hydroxyethyl is 2-hydroxy ethyl.

In any of the foregoing embodiments, the R^{1a} and/or R^{1b} C₃ alkyl is n-propyl. In any of the foregoing embodiments, the R^{1a} and/or R^{1b} C₃ alkyl is isopropyl. In any of the foregoing embodiments, the R^{1a} and/or R^{1b} C₄ alkyl is n-butyl. In any of the foregoing embodiments, the R^{1a} and/or R^{1b} C₄ alkyl is isobutyl. In any of the foregoing embodiments, the R^{1a} and/or R^{1b} C₄ alkyl is t-butyl. In any of the foregoing embodiments, the R^{1a} and/or R^{1b} C₅ alkyl is n-pentyl. In any of the foregoing embodiments, the R^{1a} and/or R^{1b} C₅ alkyl is 2-methylbutan-2-yl. In any of the foregoing embodiments, the R^{1a} and/or R^{1b} C₅ alkyl is 2,2-dimethylpropyl. In any of the foregoing embodiments, the R^{1a} and/or R^{1b} C₅ alkyl is 3-methylbutyl. In any of the foregoing embodiments, the R^{1a} and/or R^{1b} C₅ alkyl is pentan-2-yl. In any of the foregoing embodiments, the R^{1a} and/or R^{1b} C₅ alkyl is pentan-3-yl. In any of the foregoing embodiments, the R^{1a} and/or R^{1b} C₅ alkyl is 3-methylbutan-2-yl. In any of the foregoing embodiments, the R^{1a} and/or R^{1b} C₅ alkyl is 2-methylbutyl. In any of the foregoing embodiments, the R^{1a} and/or R^{1b} C₄ alkyl is branched. In any of the foregoing embodiments, the R^{1a} and/or R^{1b} C₅ alkyl is branched. In any of the foregoing embodiments, the R^{1a} and/or R^{1b} C₆ alkyl is branched.

In some embodiments, one of R^{1a} and R^{1b} is hydroxymethyl, and the other one of R^{1a} and R^{1b} is hydroxymethyl. In some embodiments, one of R^{1a} and R^{1b} is hydroxymethyl, and the other one of R^{1a} and R^{1b} is hydroxy ethyl (e.g., 1-hydroxy ethyl or 2-hydroxy ethyl). In some embodiments, one of R^{1a} and R^{1b} is hydroxymethyl, and the other one of R^{1a} and R^{1b} is 2-hydroxy-2-propyl. In some embodiments, one of R^{1a} and R^{1b} is hydroxymethyl, and the other one of R^{1a} and R^{1b} is 3-hydroxy-2-propyl. In some embodiments, one of R^{1a} and R^{1b} is hydroxymethyl, and the other one of R^{1a} and R^{1b} is 1-hydroxy-1-propyl. In some embodiments, one of R^{1a} and R^{1b} is hydroxymethyl, and the other one of R^{1a} and R^{1b} is 2-hydroxy-1-propyl. In some embodiments, one of R^{1a} and R^{1b} is hydroxymethyl, and the other one of R^{1a} and R^{1b} is 3-hydroxy-1-propyl. In some embodiments, one of R^{1a} and R^{1b} is hydroxymethyl, and the other one of R^{1a} and R^{1b} is hydroxybutyl (e.g., 4-hydroxy-1-butyl). In some embodiments, one of R^{1a} and R^{1b} is hydroxymethyl, and the other one of R^{1a} and R^{1b} is hydroxypentyl (e.g., 5-hydroxy-1-pentyl). In some embodiments, one of R^{1a} and R^{1b} is hydroxymethyl, and the other one of R^{1a} and R^{1b} is hydroxyhexyl (e.g., 6-hydroxy-1-hexyl). In some embodiments, one of R^{1a} and R^{1b} is hydroxyethyl, and the other one of R^{1a} and R^{1b} is hydroxymethyl. In some embodiments, one of R^{1a} and R^{1b} is hydroxyethyl, and the other one of R^{1a} and R^{1b} is hydroxyethyl. In some embodiments, one of R^{1a} and R^{1b} is hydroxyethyl, and the other one of R^{1a} and R^{1b} is 2-hydroxy-2-propyl. In some embodiments, one of R^{1a} and R^{1b} is hydroxyethyl, and the other one of R^{1a} and R^{1b} is 3-hydroxy-2-propyl. In some embodiments, one of R^{1a} and R^{1b} is hydroxyethyl, and the other one of R^{1a} and R^{1b} is 1-hydroxy-1-propyl. In some embodiments, one of R^{1a} and R^{1b} is hydroxyethyl, and the other one of R^{1a} and R^{1b} is 2-hydroxy-1-propyl. In some embodiments, one of R^{1a} and R^{1b} is hydroxyethyl, and the other one of R^{1a} and R^{1b} is 3-hydroxy-1-propyl. In some embodiments, one of R^{1a} and R^{1b} is hydroxyethyl, and the other one of R^{1a} and R^{1b} is hydroxybutyl. In some embodiments, one of R^{1a} and R^{1b} is hydroxyethyl, and the other one of R^{1a} and R^{1b} is hydroxypentyl. In some embodiments, one of R^{1a} and R^{1b} is hydroxyethyl, and the other one of R^{1a} and R^{1b} is hydroxyhexyl. In some embodiments, one of R^{1a} and R^{1b} is 2-hydroxy-2-propyl, and the other one of R^{1a} and R^{1b} is hydroxymethyl. In some embodiments, one of R^{1a} and R^{1b} is 2-hydroxy-2-propyl, and the other one of R^{1a} and R^{1b} is hydroxyethyl. In some embodiments, one of R^{1a} and R^{1b} is 2-hydroxy-2-propyl, and the other one of R^{1a} and R^{1b} is 2-hydroxy-2-propyl. In some embodiments, one of R^{1a} and R^{1b} is 2-hydroxy-2-propyl, and the other one of R^{1a} and R^{1b} is 3-hydroxy-2-propyl. In some embodiments, one of R^{1a} and R^{1b} is 2-hydroxy-2-propyl, and the other one of R^{1a} and R^{1b} is 1-hydroxy-1-propyl. In some embodiments, one of R^{1a} and R^{1b} is 2-hydroxy-2-propyl, and the other one of R^{1a} and R^{1b} is 2-hydroxy-1-propyl. In some embodiments, one of R^{1a} and R^{1b} is 2-hydroxy-2-propyl, and the other one of R^{1a} and R^{1b} is 3-hydroxy-1-propyl. In some embodiments, one of R^{1a} and R^{1b} is 2-hydroxy-2-propyl, and the other one of R^{1a} and R^{1b} is hydroxybutyl. In some embodiments, one of R^{1a} and R^{1b} is 2-hydroxy-2-propyl, and the other one of R^{1a} and R^{1b} is hydroxypentyl. In some embodiments, one of R^{1a} and R^{1b} is 2-hydroxy-2-propyl, and the other one of R^{1a} and R^{1b} is hydroxyhexyl.

In some embodiments, R^{1a} is C₁-C₆ alkyl substituted by one hydroxy, and R^{1b} is a C₁-C₆ alkyl substituted with one or more hydroxy, —SO₂NR¹¹R¹², —SO₂R¹³, —CONR¹¹R¹², —OR¹¹, —COR¹³; —CO₂R¹³, —NR¹³CONR¹¹R¹²; —CR¹¹R¹²CN, —NR¹¹SO₂R¹³, —NR¹¹CONR¹¹R¹², —CR¹¹R¹²NR¹¹R¹², —CN, or —NR¹¹COR¹².

In some embodiments, R^{1a} is C₁-C₆ alkyl substituted by one hydroxy, and R^{1b} is a C₁-C₆ alkyl substituted with one or more hydroxyl, —SO₂NR¹¹R¹², —SO₂R¹³, —CONR¹¹R¹², —OR¹¹, —COR¹³; —NR¹³CONR¹¹R¹²; —CR¹¹R¹²CN, —NR¹¹SO₂R¹³, —NR¹¹CONR¹¹R¹², —CR¹¹R¹²NR¹¹R¹², —CN, or —NR¹¹COR¹².

In some embodiments, R^{1a} is C₁-C₆ alkyl substituted by one hydroxy, and R^{1b} is a —SO₂NR¹¹R¹², —SO₂R¹³, —CONR¹¹R¹², —COR¹³, —CO₂R¹³, —NR¹³CONR¹¹R¹²; or —CR¹¹R¹²CN.

In some embodiments, R^{1a} is C₁-C₆ alkyl substituted by one hydroxy, and R^{1b} is a —SO₂NHMe, SO₂NHCH₂CH₂OH, SO₂Me, CONHMe, or OMe.

In some embodiments, R^{1a} is C₁-C₆ alkyl substituted by one hydroxy, and R^{1b} is a —SO₂NHMe or OMe.

In some embodiments, R^{1a} is C₁-C₆ alkyl substituted by one hydroxy, and R^{1b} is a —SO₂NH₂.

In some embodiments, R^{1a} is C₁-C₆ alkyl substituted by one hydroxy, and R^{1b} is a —SO₂NHMe.

In some embodiments, R^{1a} is C₁-C₆ alkyl substituted by one hydroxy, and R^{1b} is a —SO₂NH^{t}Bu.

In some embodiments, R^{1a} is C₁-C₆ alkyl substituted by one hydroxy, and R^{1b} —SO₂NR¹¹R¹², —SO₂R¹³, —CONR¹¹R¹², —OR¹¹, —COR¹³; —CO₂R¹³, —NR¹³CONR¹¹R¹²; —CR¹¹R¹²CN, —NR¹¹SO₂R¹³, —NR¹¹CONR¹¹R¹², CN, or —NR¹¹COR¹².

In some embodiments, R^{1a} is C₁-C₆ alkyl substituted by one hydroxy, and R^{1b} is —SO₂NR¹¹R¹².

In some embodiments, R^{1a} is C₁-C₆ alkyl substituted by one hydroxy, and R^{1b} is —SO₂R¹³.

In some embodiments, R^{1a} is C₁-C₆ alkyl substituted by one hydroxy, and R^{1b} is —CONR¹¹R¹².

In some embodiments, R^{1a} is C₁-C₆ alkyl substituted by one hydroxy, and R^{1b} is —OR¹¹.

In some embodiments, R^{1a} is C₁-C₆ alkyl substituted by one hydroxy, and R^{1b} is —COR¹³.

In some embodiments, R^{1a} is C₁-C₆ alkyl substituted by one hydroxy, and R^{1b} is —CO₂R¹³.

In some embodiments, R^{1a} is C₁-C₆ alkyl substituted by one hydroxy, and R^{1b} is —NR¹³CONR¹¹R¹².

In some embodiments, R^{1a} is C₁-C₆ alkyl substituted by one hydroxy, and R^{1b} is —CR¹¹R¹²CN.

In some embodiments, R^{1a} is C₁-C₆ alkyl substituted by one hydroxy, and R^{1b} is —NR¹¹SO₂R¹³.

In some embodiments, R^{1a} is C₁-C₆ alkyl substituted by one hydroxy, and R^{1b} is —NR¹¹CONR¹¹R¹².

In some embodiments, R^{1a} is C₁-C₆ alkyl substituted by one hydroxy, and R^{1b} is —CN.

In some embodiments, R^{1a} is C₁-C₆ alkyl substituted by one hydroxy, and R^{1b} is —NR¹¹COR¹².

In some embodiments, R^{1a} is C₁-C₆ alkyl substituted by one hydroxy, and R^{1b} is —CR¹¹R¹²NR¹¹R¹².

In some embodiments, R^{1a} is C₁-C₆ alkyl substituted by one —OSi(R¹³)₃, and R^{1b} is a C₁-C₆ alkyl substituted with one or more hydroxy, —SO₂NR¹¹R¹², —SO₂R¹³, —CONR¹¹R¹², —OR¹¹, —COR¹³; —CO₂R¹³, —NR¹³CONR¹¹R¹²; —CR¹¹R¹²CN, —NR¹¹SO₂R¹³, —NR¹¹CONR¹¹R¹², or —NR¹¹COR¹².

In some embodiments, R^{1a} is C₁-C₆ alkyl substituted by one —OSi(R¹³)₃, and R^{1b} is a C₁-C₆ alkyl substituted with one or more hydroxy, —SO₂NR¹¹R¹², —SO₂R¹³, —CONR¹¹R¹², —OR¹¹, —COR¹³; —NR¹³CONR¹¹R¹²; —CR¹¹R¹²CN, —NR¹¹SO₂R¹³, —NR¹¹CONR¹¹R¹², or —NR¹¹COR¹².

In some embodiments, R^{1a} is C₁-C₆ alkyl substituted by one —OSi(R¹³)₃, and R^{1b} is a —SO₂NR¹¹R¹², —SO₂R¹³, —CONR¹¹R¹², —COR¹³, —CO₂R¹³, —NR¹³CONR¹¹R¹²; or —CR¹¹R¹²CN.

In some embodiments, R^{1a} is C₁-C₆ alkyl substituted by one —OSi(R¹³)₃, and R^{1b} is a —SO₂NHMe, SO₂NHCH₂CH₂OH, SO₂Me, CONHMe, or OMe.

In some embodiments, R^{1a} is C₁-C₆ alkyl substituted by one —OSi(R¹³)₃, and R^{1b} is a —SO₂NHMe or OMe.

In some embodiments, R^{1a} is C₁-C₆ alkyl substituted by one —OSi(R¹³)₃, and R^{1b} —SO₂NR¹¹R¹², —SO₂R¹³, —CONR¹¹R¹², —OR¹¹, —COR¹³, —CO₂R¹³, —NR¹³CONR¹¹R¹², —CR¹¹R¹²CN, —NR¹¹SO₂R¹³, —NR¹¹CONR¹¹R¹², or —NR¹¹COR¹².

In some embodiments, R^{1a} is C₁-C₆ alkyl substituted by one —OSi(R¹³)₃, and R^{1b} is —SO₂NR¹¹R¹².

In some embodiments, R^{1a} is C₁-C₆ alkyl substituted by one —OSi(R¹³)₃, and R^{1b} is —SO₂R¹³.

In some embodiments, R^{1a} is C₁-C₆ alkyl substituted by one —OSi(R¹³)₃, and R^{1b} is —CONR¹¹R¹².

In some embodiments, R^{1a} is C₁-C₆ alkyl substituted by one —OSi(R¹³)₃, and R^{1b} is —OR¹¹.

In some embodiments, R^{1a} is C₁-C₆ alkyl substituted by one —OSi(R¹³)₃, and R^{1b} is —COR¹³.

In some embodiments, R^{1a} is C₁-C₆ alkyl substituted by one —OSi(R¹³)₃, and R^{1b} is —CO₂R¹³.

In some embodiments, R^{1a} is C₁-C₆ alkyl substituted by one —OSi(R¹³)₃, and R^{1b} is —NR¹³CONR¹¹R¹².

In some embodiments, R^{1a} is C₁-C₆ alkyl substituted by one —OSi(R¹³)₃, and R^{1b} is —CR¹¹R¹²CN.

In some embodiments, R^{1a} is C₁-C₆ alkyl substituted by one —OSi(R¹³)₃, and R^{1b} is —NR¹¹SO₂R¹³.

In some embodiments, R^{1a} is C₁-C₆ alkyl substituted by one —OSi(R¹³)₃, and R^{1b} is —NR¹¹CONR¹¹R¹².

In some embodiments, R^{1a} is C₁-C₆ alkyl substituted by one —OSi(R¹³)₃, and R^{1b} is —CN.

In some embodiments, R^{1a} is C₁-C₆ alkyl substituted by one —OSi(R¹³)₃, and R^{1b} is —NR¹¹COR¹².

In some embodiments, R^{1a} is C₁-C₆ alkyl substituted by one —OSi(R¹³)₃, and R^{1b} is —CR¹¹R¹²NR¹¹R¹².

In any of the foregoing embodiments that include —OSi(R¹³)₃, Si(R¹³)₃ is selected from trimethylsilyl, triethylsilyl, triisopropylsilyl, tert-butyldimethylsilyl, and tert-butyldiphenylsilyl.

In any of the foregoing embodiments that include —OSi(R¹³)₃, Si(R¹³)₃ is selected from tert-butyldimethyl silyl.

In some embodiments, R^{1a} is —SO₂NR¹¹R¹², and R^{1b} is a C₁-C₆ alkyl substituted with one or more hydroxy, —SO₂NR¹¹R¹², —SO₂R¹³, —CONR¹¹R¹², —OR¹¹, —COR¹³; —CO₂R¹³, —NR¹³CONR¹¹R¹²; —CR¹¹R¹²CN, —NR¹¹SO₂R¹³, —NR¹¹CONR¹¹R¹², —CR¹¹R¹²NR¹¹R¹², —CN, or —NR¹¹COR¹².

In some embodiments, R^{1a} is —SO₂NR¹¹R¹², and R^{1b} is a C₁-C₆ alkyl substituted with one or more hydroxyl, —SO₂NR¹¹R¹², —SO₂R¹³, —CONR¹¹R¹², —OR¹¹, —COR¹³; —NR¹³CONR¹¹R¹²; —CR¹¹R¹²CN, —NR¹¹SO₂R¹³, —NR¹¹CONR¹¹R¹², —CN, or —NR¹¹COR¹².

In some embodiments, R^{1a} is —SO₂NR¹¹R¹², and R^{1b} is a —SO₂NR¹¹R¹², —SO₂R¹³, —CONR¹¹R¹², —COR¹³, —CO₂R¹³, —NR¹³CONR¹¹R¹²; or —CR¹¹R¹²CN.

In some embodiments, R^{1a} is —SO₂NR¹¹R¹², and R^{1b} is a —SO₂NHMe, SO₂NHCH₂CH₂OH, SO₂Me, CONHMe, or OMe.

In some embodiments, R^{1a} is —SO₂NR¹¹R¹², and R^{1b} is a —SO₂NHMe or OMe.

In some embodiments, R^{1a} is —SO₂NR¹¹R¹², and R^{1b} is —SO₂NR¹¹R¹², —SO₂R¹³, —CONR¹¹R¹², —OR¹¹, —COR¹³; —CO₂R¹³, —NR¹³CONR¹¹R¹²; —CR¹¹R¹²CN, —NR¹¹SO₂R¹³, —NR¹¹CONR¹¹R¹², or —NR¹¹COR¹².

In some embodiments, R^{1a} is C₁-C₆ alkyl substituted by one hydroxy, and R^{1b} is —SO₂NR¹¹R¹².

In some embodiments, R^{1a} is —SO₂NR¹¹R¹², and R^{1b} is —SO₂R¹³. In some embodiments, R^{1a} is —SO₂NR¹¹R¹², and R^{1b} is —CONR¹¹R¹². In some embodiments, R^{1a} is —SO₂NR¹¹R¹², and R^{1b} is —OR¹¹. In some embodiments, R^{1a} is —SO₂NR¹¹R¹², and R^{1b} is —COR¹³. In some embodiments, R^{1a} is —SO₂NR¹¹R¹², and R^{1b} is —CO₂R¹³. In some embodiments, R^{1a} is —SO₂NR¹¹R¹², and R^{1b} is —NR¹³CONR¹¹R¹². In some embodiments, R^{1a} is —SO₂NR¹¹R¹², and R^{1b} is —CR¹¹R¹²CN. In some embodiments, R^{1a} is —SO₂NR¹¹R¹², and R^{1b} is —NR¹¹SO₂R¹³. In some embodiments, R^{1a} is —SO₂NR¹¹R¹², and R^{1b} is —NR¹¹CONR¹¹R¹². In some embodiments, R^{1a} is —SO₂NR¹¹R¹², and R^{1b} is —CN. In some embodiments, R^{1a} is —SO₂NR¹¹R¹², and R^{1b} is —NR¹¹COR¹². In some embodiments, R^{1a} is —SO₂NR¹¹R¹², and R^{1b} is —CR¹¹R¹²NR¹¹R¹².

In some embodiments, R^{1a} is 2-hydroxy-2-propyl, and R^{1b} is —OMe. In some embodiments, R^{1a} is 2-hydroxy-2-propyl, and R^{1b} is —OH. In some embodiments, R^{1a} is 2-hydroxy-2-propyl, and R^{1b} is —CO₂Me. In some embodiments, R^{1a} is 2-hydroxy-2-propyl, and R^{1b} is hydroxymethyl. In some embodiments, R^{1a} is 2-hydroxy-2-propyl, and R^{1b} is hydroxy ethyl. In some embodiments, R^{1a} is 2-hydroxy-2-propyl, and R^{1b} is 2-hydroxy-2-propyl. In some embodiments, R^{1a} is 2-hydroxy-2-propyl, and R^{1b} is —SO₂NHCH₂CH₂OH. In some embodiments, R^{1a} is 2-hydroxy-2-propyl, and R^{1b} is —SO₂NH₂. In some embodiments, R^{1a} is 2-hydroxy-2-propyl, and R^{1b} is —SO₂NHMe. In some embodiments, R^{1a} is 2-hydroxy-2-propyl, and R^{1b} is —SO₂NH^{t}Bu.

In some embodiments, R^{1a} is 2-hydroxy-2-propyl, and R^{1b} is —SO₂Me. In some embodiments, R^{1a} is 2-hydroxy-2-propyl, and R^{1b} is CONHMe. In some embodiments, R^{1a} is 2-hydroxy-2-propyl, and R^{1b} is cyanomethyl. In some embodiments, R^{1a} is 2-hydroxy-2-propyl, and R^{1b} is dimethylaminomethyl. In some embodiments, R^{1a} is —SO₂NHMe, and R^{1b} is —OMe. In some embodiments, R^{1a} is —SO₂NHMe, and R^{1b} is —OH. In some embodiments, R^{1a} is —SO₂NHMe, and R^{1b} is —CO₂Me. In some embodiments, R^{1a} is —SO₂NHMe, and R^{1b} is —CO₂H. In some embodiments, R^{1a} is —SO₂NHMe, and R^{1b} is —CN. In some embodiments, R^{1a} is —SO₂NHMe, and R^{1b} is hydroxymethyl. In some embodiments, R^{1a} is —SO₂NHMe, and R^{1b} is hydroxyethyl. In some embodiments, R^{1a} is —SO₂NHMe, and R^{1b} is 2-hydroxy-2-propyl. In some embodiments, R^{1a} is —SO₂NHMe, and R^{1b} is —SO₂NHCH₂CH₂OH. In some embodiments, R^{1a} is —SO₂NHMe, and R^{1b} is —SO₂Me. In some embodiments, R^{1a} is —SO₂NHMe, and R^{1b} is CONHMe. In some embodiments, R^{1a} is —SO₂NHMe, and R^{1b} is CONMe₂. In some embodiments, R^{1a} is —SO₂NHMe, and R^{1b} is CONH₂. In some embodiments, R^{1a} is —SO₂NHMe, and R^{1b} is cyanomethyl. In some embodiments, R^{1a} is —SO₂NHMe, and R^{1b} is dimethylaminomethyl. In some embodiments, R^{1a} is —SO₂NH^{t}Bu, and R^{1b} is hydroxymethyl. In some embodiments, R^{1a} is —SO₂NH₂, and R^{1b} is hydroxymethyl. In some embodiments, R^{1a} is —SO₂NHCH₂CH₂OH, and R^{1b} is OMe.

In some embodiments, R^{1a} is C₁-C₄ alkyl substituted with one —OSi(Me)₂tBu, and R^{1b} is —CO₂Me.

In some embodiments, R^{1a} is —CR¹¹R¹²NR¹¹R¹², and R^{1b} is a C₁-C₆ alkyl substituted with one or more hydroxy, —SO₂NR¹¹R¹², —SO₂R¹³, —CONR¹¹R¹², —OR¹¹, —COR¹³; —CO₂R¹³, —NR¹³CONR¹¹R¹²; —CR¹¹R¹²CN, —NR¹¹SO₂R¹³, —NR¹¹CONR¹¹R¹², —CR¹¹R¹²NR¹¹R¹², —CN, or —NR¹¹COR¹².

In some embodiments, R^{1a} is —CR¹¹R¹²NR¹¹R¹², and R^{1b} is a C₁-C₆ alkyl substituted with one or more hydroxyl, —SO₂NR¹¹R¹², —SO₂R¹³, —CONR¹¹R¹², —OR¹¹, —COR¹³; —NR¹³CONR¹¹R¹²; —CR¹¹R¹²CN, —NR¹¹SO₂R¹³, —NR¹¹CONR¹¹R¹², or —NR¹¹COR¹².

In some embodiments, R^{1a} is —CR¹¹R¹²NR¹¹R¹², and R^{1b} is a —SO₂NR¹¹R¹², —SO₂R¹³, —CONR¹¹R¹², —COR¹³, —CO₂R¹³, —NR¹³CONR¹¹R¹²; or —CR¹¹R¹²CN.

In some embodiments, R^{1a} is —CR¹¹R¹²NR¹¹R¹², and R^{1b} is a —SO₂NHMe, SO₂NHCH₂CH₂OH, SO₂Me, CONHMe, or OMe.

In some embodiments, R^{1a} is —CR¹¹R¹²NR¹¹R¹², and R^{1b} is a a C₁-C₆ alkyl substituted with one or more hydroxyl.

In some embodiments, R^{1a} is —CR¹¹R¹²NR¹¹R¹², and R^{1b} is —SO₂NR¹¹R¹², —SO₂R¹³, —CONR¹¹R¹², —OR¹¹, —COR¹³; —CO₂R¹³, —NR¹³CONR¹¹R¹²; —CR¹¹R¹²CN, —NR¹¹SO₂R¹³, —NR¹¹CONR¹¹R¹², or —NR¹¹COR¹².

In some embodiments, R^{1a} is —CR¹¹R¹²NR¹¹R¹², and R^{1b} is —SO₂R¹³. In some embodiments, R^{1a} is —CR¹¹R¹²NR¹¹R¹², and R^{1b} is —SO₂R¹³. In some embodiments, R^{1a} is —CR¹¹R¹²NR¹¹R¹², and R^{1b} is —CONR¹¹R¹². In some embodiments, R^{1a} is —CR¹¹R¹²NR¹¹R¹², and R^{1b} is —OR¹¹. In some embodiments, R^{1a} is —CR¹¹R¹²NR¹¹R¹², and R^{1b} is —COR¹³. In some embodiments, R^{1a} is —CR¹¹R¹²NR¹¹R¹², and R^{1b} is —CO₂R¹³. In some embodiments, R^{1a} is —CR¹¹R¹²NR¹¹R¹², and R^{1b} is —NR¹³CONR¹¹R¹². In some embodiments, R^{1a} is —CR¹¹R¹²NR¹¹R¹², and R^{1b} is —CR¹¹R¹²CN. In some embodiments, R^{1a} is —CR¹¹R¹²NR¹¹R¹², and R^{1b} is —NR¹¹SO₂R¹³. In some embodiments, R^{1a} is —CR¹¹R¹²NR¹¹R¹², and R^{1b} is —NR¹¹CONR¹¹R¹². In some embodiments, R^{1a} is —CR¹¹R¹²NR¹¹R¹², and R^{1b} is —CN. In some embodiments, R^{1a} is —CR¹¹R¹²NR¹¹R¹², and R^{1b} is —NR¹¹COR¹². In some embodiments, R^{1a} is —CR¹¹R¹²NR¹¹R¹², and R^{1b} is —CR¹¹R¹²NR¹¹R¹².

In some embodiments, R^{1a} is dimethylaminomethyl, and R^{1b} is —OMe. In some embodiments, R^{1a} is dimethylaminomethyl, and R^{1b} is —OH. In some embodiments, R^{1a} is dimethylaminomethyl, and R^{1b} is —CO₂Me. In some embodiments, R^{1a} is dimethylaminomethyl, and R^{1b} is hydroxymethyl. In some embodiments, R^{1a} is dimethylaminomethyl, and R^{1b} is hydroxyethyl. In some embodiments, R^{1a} is dimethylaminomethyl, and R^{1b} is 2-hydroxy-2-propyl. In some embodiments, R^{1a} is dimethylaminomethyl, and R^{1b} is —SO₂NHCH₂CH₂OH. In some embodiments, R^{1a} is dimethylaminomethyl, and R^{1b} is —SO₂Me. In some embodiments, R^{1a} is dimethylaminomethyl, and R^{1b} is —CN. In some embodiments, R^{1a} is dimethylaminomethyl, and R^{1b} is CONHMe. In some embodiments, R^{1a} is dimethylaminomethyl, and R^{1b} is cyanomethyl. In some embodiments, R^{1a} is dimethylaminomethyl, and R^{1b} is dimethylaminomethyl.

In some embodiments, R^{1a′} is C₁-C₆ alkyl substituted by one —OSi(R¹³)₃, and R^{1b} is a C₁-C₆ alkyl substituted with one or more hydroxy, —SO₂NR¹¹R¹², —SO₂R¹³, —CONR¹¹R¹², —OR¹¹, —COR¹³; —CO₂R¹³, —NR¹³CONR¹¹R¹²; —CR¹¹R¹²CN, —NR¹¹SO₂R¹³, —NR¹¹CONR¹¹R¹², —CN, or —NR¹¹COR¹².

In some embodiments, R^{1a′} is C₁-C₆ alkyl substituted by one —OSi(R¹³)₃, and R^{1b} is a C₁-C₆ alkyl substituted with one or more hydroxy, —SO₂NR¹¹R¹², —SO₂R¹³, —CONR¹¹R¹², —OR¹¹, —COR¹³; —NR¹³CONR¹¹R¹²; —CR¹¹R¹²CN, —NR¹¹SO₂R¹³, —NR¹¹CONR¹¹R¹², or —NR¹¹COR¹².

In some embodiments, R^{1a′} is C₁-C₆ alkyl substituted by one —OSi(R¹³)₃, and R^{1b} is a —SO₂NR¹¹R¹², —SO₂R¹³, —CONR¹¹R¹², —COR¹³, —CO₂R¹³, —NR¹³CONR¹¹R¹²; or —CR¹¹R¹²CN.

In some embodiments, R^{1a′} is C₁-C₆ alkyl substituted by one —OSi(R¹³)₃, and R^{1b} is a —SO₂NHMe, SO₂NHCH₂CH₂OH, SO₂Me, CONHMe, or OMe.

In some embodiments, R^{1a′} is C₁-C₆ alkyl substituted by one —OSi(R¹³)₃, and R^{1b} is a —SO₂NHMe or OMe.

In some embodiments, R^{1a′} is C₁-C₆ alkyl substituted by one —OSi(R¹³)₃, and R^{1b} —SO₂NR¹¹R¹², —SO₂R¹³, —CONR¹¹R¹², —OR¹¹, —COR¹³, —CO₂R¹³, —NR¹³CONR¹¹R¹², —CR¹¹R¹²CN, —NR¹¹SO₂R¹³, —NR¹¹CONR¹¹R¹², or —NR¹¹COR¹².

In some embodiments, R^{1a′} is C₁-C₆ alkyl substituted by one —OSi(R¹³)₃, and R^{1b} is —SO₂NR¹¹R¹². In some embodiments, R^{1a′} is C₁-C₆ alkyl substituted by one —OSi(R¹³)₃, and R^{1b} is —SO₂R¹³. In some embodiments, R^{1a′} is C₁-C₆ alkyl substituted by one —OSi(R¹³)₃, and R^{1b} is —CONR¹¹R¹². In some embodiments, R^{1a′} is C₁-C₆ alkyl substituted by one —OSi(R¹³)₃, and R^{1b} is —OR¹¹. In some embodiments, R^{1a′} is C₁-C₆ alkyl substituted by one —OSi(R¹³)₃, and R^{1b} is —COR¹³. In some embodiments, R^{1a′} is C₁-C₆ alkyl substituted by one —OSi(R¹³)₃, and R^{1b} is —CO₂R¹³. In some embodiments, R^{1a′} is C₁-C₆ alkyl substituted by one —OSi(R¹³)₃, and R^{1b} is —NR¹³CONR¹¹R¹². In some embodiments, R^{1a′} is C₁-C₆ alkyl substituted by one —OSi(R¹³)₃, and R^{1b} is —CR¹¹R¹²CN. In some embodiments, R^{1a′} is C₁-C₆ alkyl substituted by one —OSi(R¹³)₃, and R^{1b} is —NR¹¹SO₂R¹³. In some embodiments, R^{1a′} is C₁-C₆ alkyl substituted by one —OSi(R¹³)₃, and R^{1b} is —NR¹¹CONR¹¹R¹². In some embodiments, R^{1a′} is C₁-C₆ alkyl substituted by one —OSi(R¹³)₃, and R^{1b} is —CN. In some embodiments, R^{1a′} is C₁-C₆ alkyl substituted by one —OSi(R¹³)₃, and R^{1b} is —NR¹¹COR¹². In some embodiments, R^{1a′} is C₁-C₆ alkyl substituted by one —OSi(R¹³)₃, and R^{1b} is —CR¹¹R¹²NR¹¹R¹².

In any of the foregoing embodiments that include —OSi(R¹³)₃, Si(R¹³)₃ is selected from trimethylsilyl, triethylsilyl, triisopropylsilyl, tert-butyldimethylsilyl, and tert-butyldiphenylsilyl.

In any of the foregoing embodiments that include —OSi(R¹³)₃, Si(R¹³)₃ is selected from tert-butyldimethyl silyl.

In some embodiments, R^{1a′} is —SO₂NR¹¹R¹², and R^{1b} is a C₁-C₆ alkyl substituted with one or more hydroxy, —SO₂NR¹¹R¹², —SO₂R¹³, —CONR¹¹R¹², —OR¹¹, —COR¹³; —CO₂R¹³, —NR¹³CONR¹¹R¹²; —CR¹¹R¹²CN, —NR¹¹SO₂R¹³, —NR¹¹CONR¹¹R¹², —CR¹¹R¹²NR¹¹R¹², CN, or —NR¹¹COR¹².

In some embodiments, R^{1a′} is —SO₂NR¹¹R¹², and R^{1b} is a C₁-C₆ alkyl substituted with one or more hydroxyl, —SO₂NR¹¹R¹², —SO₂R¹³, —CONR¹¹R¹², —OR¹¹, —COR¹³; —NR¹³CONR¹¹R¹²; —CR¹¹R¹²CN, —NR¹¹SO₂R¹³, —NR¹¹CONR¹¹R¹², or —NR¹¹COR¹².

In some embodiments, R^{1a′} is —SO₂NR¹¹R¹², and R^{1b} is a —SO₂NR¹¹R¹², —SO₂R¹³, —CONR¹¹R¹², —COR¹³, —CO₂R¹³, —NR¹³CONR¹¹R¹²; or —CR¹¹R¹²CN.

In some embodiments, R^{1a′} is —SO₂NR¹¹R¹², and R^{1b} is a —SO₂NHMe, SO₂NHCH₂CH₂OH, SO₂Me, CONHMe, or OMe.

In some embodiments, R^{1a′} is —SO₂NR¹¹R¹², and R^{1b} is a —SO₂NHMe or OMe.

In some embodiments, R^{1a′} is —SO₂NR¹¹R¹², and R^{1b} is —SO₂NR¹¹R¹², —SO₂R¹³, —CONR¹¹R¹², —OR¹¹, —COR¹³; —CO₂R¹³, —NR¹³CONR¹¹R¹²; —CR¹¹R¹²CN, —NR¹¹SO₂R¹³, —NR¹¹CONR¹¹R¹², or —NR¹¹COR¹².

In some embodiments, R^{1a′} is —SO₂NR¹¹R¹², and R^{1b} is —SO₂R¹³. In some embodiments, R^{1a′} is —SO₂NR¹¹R¹², and R^{1b} is —CONR¹¹R¹². In some embodiments, R^{1a′} is —SO₂NR¹¹R¹², and R^{1b} is —OR¹¹. In some embodiments, R^{1a′} is —SO₂NR¹¹R¹², and R^{1b} is —COR¹³. In some embodiments, R^{1a′} is —SO₂NR¹¹R¹², and R^{1b} is —CO₂R¹³. In some embodiments, R^{1a′} is —SO₂NR¹¹R¹², and R^{1b} is —NR¹³CONR¹¹R¹². In some embodiments, R^{1a′} is —SO₂NR¹¹R¹², and R^{1b} is —CR¹¹R¹²CN. In some embodiments, R^{1a′} is —SO₂NR¹¹R¹², and R^{1b} is —NR¹¹SO₂R¹³. In some embodiments, R^{1a′} is —SO₂NR¹¹R¹², and R^{1b} is —NR¹¹CONR¹¹R¹². In some embodiments, R^{1a′} is —SO₂NR¹¹R¹², and R^{1b} is —CN. In some embodiments, R^{1a′} is —SO₂NR¹¹R¹², and R^{1b} is —NR¹¹COR¹². In some embodiments, R^{1a′} is —SO₂NR¹¹R¹², and R^{1b} is —CR¹¹R¹²NR¹¹R¹².

In some embodiments, R^{1a} is —SO₂NHMe, and R^{1b} is —OMe. In some embodiments, R^{1a′} is —SO₂NHMe, and R^{1b} is —OH. In some embodiments, R^{1a′} is —SO₂NHMe, and R^{1b} is —CO₂Me. In some embodiments, R^{1a′} is —SO₂NHMe, and R^{1b} is —CO₂H. In some embodiments, R^{1a′} is —SO₂NHMe, and R^{1b} is hydroxymethyl. In some embodiments, R^{1a′} is —SO₂NHMe, and R^{1b} is hydroxyethyl. In some embodiments, R^{1a′} is —SO₂NHMe, and R^{1b} is 2-hydroxy-2-propyl. In some embodiments, R^{1a′} is —SO₂NHMe, and R^{1b} is —SO₂NHCH₂CH₂OH. In some embodiments, R^{1a′} is —SO₂NHMe, and R^{1b} is —SO₂Me. In some embodiments, R^{1a′} is —SO₂NHMe, and R^{1b} is CONHMe. In some embodiments, R^{1a′} is —SO₂NHMe, and R^{1b} is CONH₂. In some embodiments, R^{1a′} is —SO₂NHMe, and R^{1b} is CONMe₂. In some embodiments, R^{1a′} is —SO₂NHMe, and R^{1b} is cyanomethyl. In some embodiments, R^{1a′} is —SO₂NHMe, and R^{1b} is —CN.

In some embodiments, R^{1a′} is —SO₂NHMe, and R^{1b} is dimethylaminomethyl. In some embodiments, R^{1a′} is —SO₂NH₂, and R^{1b} is hydroxymethyl. In some embodiments, R^{1a} is —SO₂NH^{t}Bu, and R^{1b} is hydroxymethyl. In some embodiments, R^{1a′} is —SO₂NHCH₂CH₂OH, and R^{1b} is OMe.

In some embodiments, R^{1a′} is C₁-C₄ alkyl substituted with one —OSi(Me)₂tBu, and R^{1b} is —CO₂Me.

In some embodiments, R^{1a′} is —CR¹¹R¹²NR¹¹R¹², and R^{1b} is a C₁-C₆ alkyl substituted with one or more hydroxy, —SO₂NR¹¹R¹², —SO₂R¹³, —CONR¹¹R¹², —OR¹¹, —COR¹³; —CO₂R¹³, —NR¹³CONR¹¹R¹²; —CR¹¹R¹²CN, —NR¹¹SO₂R¹³, —NR¹¹CONR¹¹R¹², —CR¹¹R¹²NR¹¹R¹², CN, or —NR¹¹COR¹².

In some embodiments, R^{1a′} is —CR¹¹R¹²NR¹¹R¹², and R^{1b} is a C₁-C₆ alkyl substituted with one or more hydroxyl, —SO₂NR¹¹R¹², —SO₂R¹³, —CONR¹¹R¹², —OR¹¹, —COR¹³; —NR¹³CONR¹¹R¹²; —CR¹¹R¹²CN, —NR¹¹SO₂R¹³, —NR¹¹CONR¹¹R¹², or —NR¹¹COR¹².

In some embodiments, R^{1a′} is —CR¹¹R¹²NR¹¹R¹², and R^{1b} is a —SO₂NR¹¹R¹², —SO₂R¹³, —CONR¹¹R¹², —COR¹³, —CO₂R¹³, —NR¹³CONR¹¹R¹²; or —CR¹¹R¹²CN.

In some embodiments, R^{1a′} is —CR¹¹R¹²NR¹¹R¹², and R^{1b} is a —SO₂NHMe, SO₂NHCH₂CH₂OH, SO₂Me, CONHMe, or OMe.

In some embodiments, R^{1a′} is —CR¹¹R¹²NR¹¹R¹², and R^{1b} is a —SO₂NHMe or OMe.

In some embodiments, R^{1a′} is —CR¹¹R¹²NR¹¹R¹², and R^{1b} is —SO₂NR¹¹R¹², —SO₂R¹³, —CONR¹¹R¹², —OR¹¹, —COR¹³; —CO₂R¹³, —NR¹³CONR¹¹R¹²; —CR¹¹R¹²CN, —NR¹¹SO₂R¹³, —NR¹¹CONR¹¹R¹², or —NR¹¹COR¹².

In some embodiments, R^{1a′} is —CR¹¹R¹²NR¹¹R¹², and R^{1b} is —SO₂R¹³. In some embodiments, R^{1a′} is —CR¹¹R¹²NR¹¹R¹², and R^{1b} is —CONR¹¹R¹². In some embodiments, R^{1a′} is —CR¹¹R¹²NR¹¹R¹², and R^{1b} is —OR¹¹. In some embodiments, R^{1a′} is —CR¹¹R¹²NR¹¹R¹², and R^{1b} is —COR¹³. In some embodiments, R^{1a′} is —CR¹¹R¹²NR¹¹R¹², and R^{1b} is —CO₂R¹³. In some embodiments, R^{1a′} is —CR¹¹R¹²NR¹¹R¹², and R^{1b} is —NR¹³CONR¹¹R¹². In some embodiments, R^{1a′} is —CR¹¹R¹²NR¹¹R¹², and R^{1b} is —CR¹¹R¹²CN. In some embodiments, R^{1a′} is —CR¹¹R¹²NR¹¹R¹², and R^{1b} is —NR¹¹SO₂R¹³. In some embodiments, R^{1a′} is —CR¹¹R¹²NR¹¹R¹², and R^{1b} is —NR¹¹CONR¹¹R¹². In some embodiments, R^{1a′} is —CR¹¹R¹²NR¹¹R¹², and R^{1b} is —CN. In some embodiments, R^{1a′} is —CR¹¹R¹²NR¹¹R¹², and R^{1b} is —NR¹¹COR¹². In some embodiments, R^{1a} is —CR¹¹R¹²NR¹¹R¹², and R^{1b} is —CR¹¹R¹²NR¹¹R¹².

In some embodiments, R^{1a′} is dimethylaminomethyl, and R^{1b} is —OMe. In some embodiments, R^{1a′} is dimethylaminomethyl, and R^{1b} is —OH. In some embodiments, R^{1a′} is dimethylaminomethyl, and R^{1b} is —CO₂Me. In some embodiments, R^{1a′} is dimethylaminomethyl, and R^{1b} is hydroxymethyl. In some embodiments, R^{1a′} is dimethylaminomethyl, and R^{1b} is hydroxyethyl. In some embodiments, R^{1a′} is dimethylaminomethyl, and R^{1b} is 2-hydroxy-2-propyl. In some embodiments, R^{1a′} is dimethylaminomethyl, and R^{1b} is —SO₂NHCH₂CH₂OH. In some embodiments, R^{1a′} is dimethylaminomethyl, and R^{1b} is —SO₂Me. In some embodiments, R^{1a′} is dimethylaminomethyl, and R^{1b} is CONHMe. In some embodiments, R^{1a′} is dimethylaminomethyl, and R^{1b} is —CN. In some embodiments, R^{1a′} is dimethylaminomethyl, and R^{1b} is cyanomethyl. In some embodiments, R^{1a} is dimethylaminomethyl, and R^{1b} is dimethylaminomethyl.

In some embodiments, R^{1a″} is C₁-C₆ alkyl substituted by one hydroxy, and R^{1b′} is —SO₂NR¹¹R¹², —SO₂R¹³, —CONR¹¹R¹², —OR¹¹, —COR¹³; —CO₂R¹³, —NR¹³CONR¹¹R¹²; —CR¹¹R¹²CN, —NR¹¹SO₂R¹³, —NR¹¹CONR¹¹R¹², —CR¹¹R¹²NR¹¹R¹², —CN, or —NR¹¹COR¹².

In some embodiments, R^{1a″} is C₁-C₆ alkyl substituted by one hydroxy, and R^{1b′} is —SO₂NR¹¹R¹², —SO₂R¹³, —CONR¹¹R¹², —OR¹¹, —COR¹³; —NR¹³CONR¹¹R¹²; —CR¹¹R¹²CN, —NR¹¹SO₂R¹³, —NR¹¹CONR¹¹R¹², —CR¹¹R¹²NR¹¹R¹², —CN, or —NR¹¹COR¹².

In some embodiments, R^{1a″} is C₁-C₆ alkyl substituted by one hydroxy, and R^{1b′} is a —SO₂NR¹¹R¹², —SO₂R¹³, —CONR¹¹R¹², —COR¹³, —CO₂R¹³, —NR¹³CONR¹¹R¹²; or —CR¹¹R¹²CN.

In some embodiments, R^{1a″} is C₁-C₆ alkyl substituted by one hydroxy, and R^{1b′} is a —SO₂NHMe, SO₂NHCH₂CH₂OH, SO₂Me, CONHMe, or OMe.

In some embodiments, R^{1a″} is C₁-C₆ alkyl substituted by one hydroxy, and R^{1b′} is a —SO₂NHMe or OMe.

In some embodiments, R^{1a″} is C₁-C₆ alkyl substituted by one hydroxy, and R^{1b′} —SO₂NR¹¹R¹², —SO₂R¹³, —CONR¹¹R¹², —OR¹¹, —COR¹³; —CO₂R¹³, —NR¹³CONR¹¹R¹²; —CR¹¹R¹²CN, —NR¹¹SO₂R¹³, —NR¹¹CONR¹¹R¹², CN, or —NR¹¹COR¹².

In some embodiments, R^{1a″} is C₁-C₆ alkyl substituted by one hydroxy, and R^{1b′} is —SO₂NR¹¹R¹².

In some embodiments, R^{1a″} is C₁-C₆ alkyl substituted by one hydroxy, and R^{1b} is —SO₂R¹³. In some embodiments, R^{1a″} is C₁-C₆ alkyl substituted by one hydroxy, and R^{1b′} is —CONR¹¹R¹². In some embodiments, R^{1a″} is C₁-C₆ alkyl substituted by one hydroxy, and R^{1b′} is —OR¹¹. In some embodiments, R^{1a″} is C₁-C₆ alkyl substituted by one hydroxy, and R^{1b′} is —COR¹³. In some embodiments, R^{1a″} is C₁-C₆ alkyl substituted by one hydroxy, and R^{1b′} is —CO₂R¹³. In some embodiments, R^{1a″} is C₁-C₆ alkyl substituted by one hydroxy, and R^{1b′} is —NR¹³CONR¹¹R¹². In some embodiments, R^{1a″} is C₁-C₆ alkyl substituted by one hydroxy, and R^{1b′} is —CR¹¹R¹²CN. In some embodiments, R^{1a″} is C₁-C₆ alkyl substituted by one hydroxy, and R^{1b′} is —NR¹¹SO₂R¹³. In some embodiments, R^{1a″} is C₁-C₆ alkyl substituted by one hydroxy, and R^{1b′} is —NR¹¹CONR¹¹R¹². In some embodiments, R^{1a″} is C₁-C₆ alkyl substituted by one hydroxy, and R^{1b′} is —CN. In some embodiments, R^{1a″} is C₁-C₆ alkyl substituted by one hydroxy, and R^{1b′} is —NR¹¹COR¹². In some embodiments, R^{1a″} is C₁-C₆ alkyl substituted by one hydroxy, and R^{1b′} is —CR¹¹R¹²NR¹¹R¹².

In some embodiments, R^{1a″} is 2-hydroxy-2-propyl, and R^{1b′} is —OMe. In some embodiments, R^{1a″} is 2-hydroxy-2-propyl, and R^{1b′} is —OH. In some embodiments, R^{1a″} is 2-hydroxy-2-propyl, and R^{1b′} is —CO₂Me. In some embodiments, R^{1a″} is 2-hydroxy-2-propyl, and R^{1b′} is —SO₂NHCH₂CH₂OH. In some embodiments, R^{1a″} is 2-hydroxy-2-propyl, and R^{1b′} is —SO₂Me. In some embodiments, R^{1a″} is 2-hydroxy-2-propyl, and R^{1b′} is CONHMe. In some embodiments, R^{1a″} is 2-hydroxy-2-propyl, and R^{1b′} is cyanomethyl. In some embodiments, R^{1a″} is 2-hydroxy-2-propyl, and R^{1b′} is dimethylaminomethyl.

In some embodiments, R^{1a″} is 2-hydroxy-2-propyl, and R^{1b} is hydroxymethyl.

### The Group R²

In some embodiments,

R² is selected from C₁-C₆ alkyl, C₁-C₆ haloalkyl, C₁-C₆ alkoxy, C₁-C₆ haloalkoxy, halo, CN, NO₂, COC₁-C₆ alkyl, CO—C₆-C₁₀ aryl, CO(5- to 10-membered heteroaryl), CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, S(O₂)NR¹¹R¹², S(O)C₁-C₆ alkyl, C₃-C₇ cycloalkyl and 3- to 7-membered heterocycloalkyl,

wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), and OCO(3- to 7-membered heterocycloalkyl);
wherein each C₁-C₆ alkyl substituent and each C₁-C₆, alkoxy substituent of the R² C₃-C₇ cycloalkyl or of the R² 3- to 7-membered heterocycloalkyl is further optionally independently substituted with one to three hydroxy, halo, or oxo;
wherein the 3- to 7-membered heterocycloalkyl, C₆-C₁₀ aryl, and 5- to 10-membered heteroaryl of the R² C₁-C₆ alkyl, the R² C₁-C₆ haloalkyl, the R² C₃-C₇ cycloalkyl, or the R² 3- to 7-membered heterocycloalkyl are optionally substituted with one or more substituents independently selected from halo, C₁-C₆ alkyl, and OC₁-C₆ alkyl.

In some embodiments,

R² is selected from C₁-C₆ haloalkyl, C₁-C₆ alkoxy, C₁-C₆ haloalkoxy, halo, CN, NO₂, COC₁-C₆ alkyl, CO—C₆-C₁₀ aryl, CO(5- to 10-membered heteroaryl), CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, S(O₂)NR¹¹R¹², S(O)C₁-C₆ alkyl, C₃-C₇ cycloalkyl and 3- to 7-membered heterocycloalkyl,
wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), and OCO(3- to 7-membered heterocycloalkyl);
wherein each C₁-C₆ alkyl substituent and each C₁-C₆ alkoxy substituent of the R² C₃-C₇ cycloalkyl or of the R² 3- to 7-membered heterocycloalkyl is further optionally independently substituted with one to three hydroxy, halo, or oxo;
wherein the 3- to 7-membered heterocycloalkyl, C₆-C₁₀ aryl, 5- to 10-membered heteroaryl of the R² C₁-C₆ alkyl, the R² C₁-C₆ haloalkyl, the R² C₃-C₇ cycloalkyl, or the R² 3- to 7-membered heterocycloalkyl are optionally substituted with one or more substituents independently selected from halo, C₁-C₆ alkyl, and OC₁-C₆ alkyl.

In some embodiments,

R² is selected from C₁-C₆ alkyl, halo, CN, NO₂, COC₁-C₆ alkyl, CO—C₆-C₁₀ aryl, CO(5- to 10-membered heteroaryl), CO₂C₁-C₆ alkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₃-C₆ alkyl, S(O₂)NR¹¹R¹², S(O₂)C₁-C₆ alkyl, C₃-C₇ cycloalkyl and 3- to 7-membered heterocycloalkyl,
wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), and OCO(3- to 7-membered heterocycloalkyl);
wherein each C₁-C₆ alkyl substituent and each C₁-C₆ alkoxy substituent of the R² C₃-C₇ cycloalkyl or of the R² 3- to 7-membered heterocycloalkyl is further optionally independently substituted with one to three hydroxy, halo, or oxo;
wherein the 3- to 7-membered heterocycloalkyl, C₆-C₁₀ aryl, 5- to 10-membered heteroaryl of the R² C₁-C₆ alkyl, the R² C₁-C₆ haloalkyl, the R² C₃-C₇ cycloalkyl, or the R² 3- to 7-membered heterocycloalkyl are optionally substituted with one or more substituents independently selected from halo, C₁-C₆ alkyl, and OC₁-C₆ alkyl.

In some embodiments,

R² is selected from C₁-C₆ alkyl, C₁-C₆ haloalkyl, C₁-C₆ alkoxy, C₁-C₆ haloalkoxy, halo, CN, NO₂, COC₁-C₆ alkyl, CO—C₆-C₁₀ aryl, CO(5- to 10-membered heteroaryl), CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₃-C₆ alkyl, S(O₂)NR¹¹R¹², S(O)C₁-C₆ alkyl, C₃-C₇ cycloalkyl and 3- to 7-membered heterocycloalkyl,
wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, OCOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), and OCO(3- to 7-membered heterocycloalkyl);
wherein each C₁-C₆ alkyl substituent and each C₁-C₆ alkoxy substituent of the R² C₃-C₇ cycloalkyl or of the R² 3- to 7-membered heterocycloalkyl is further optionally independently substituted with one to three hydroxy, halo, or oxo;
wherein the 3- to 7-membered heterocycloalkyl, C₆-C₁₀ aryl, 5- to 10-membered heteroaryl of the R² C₁-C₆ alkyl, the R² C₁-C₆ haloalkyl, the R² C₃-C₇ cycloalkyl, or the R² 3- to 7-membered heterocycloalkyl are optionally substituted with one or more substituents independently selected from halo, C₁-C₆ alkyl, and OC₁-C₆ alkyl;

In some embodiments,

R² is selected from C₁-C₆ alkyl, C₁-C₆ haloalkyl, C₁-C₆ alkoxy, C₁-C₆ haloalkoxy, halo, CN, NO₂, COC₁-C₆ alkyl, CO—C₆-C₁₀ aryl, CO(5- to 10-membered heteroaryl), CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, S(O₂)NR¹¹R¹², S(O)C₁-C₆ alkyl, C₃-C₇ cycloalkyl and 3- to 7-membered heterocycloalkyl, wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl);
wherein the 3- to 7-membered heterocycloalkyl, C₆-C₁₀ aryl, or 5- to 10-membered heteroaryl of the R² C₁-C₆ alkyl, the R² C₁-C₆ haloalkyl, the R² C₃-C₇ cycloalkyl, or the R² 3- to 7-membered heterocycloalkyl are optionally substituted with one or more substituents independently selected from halo, C₁-C₆ alkyl, and OC₁-C₆ alkyl.

In some embodiments,

R² is selected from C₁-C₆ alkyl, C₁-C₆ haloalkyl, C₁-C₆ alkoxy, C₁-C₆ haloalkoxy, halo, CN, NO₂, COC₁-C₆ alkyl, CO—C₆-C₁₀ aryl, CO(5- to 10-membered heteroaryl), CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, S(O₂)NR¹¹R¹², S(O)C₁-C₆ alkyl, C₃-C₇ cycloalkyl and 3- to 7-membered heterocycloalkyl, wherein the C₁-C₆ alkyl, C₁-C₆ haloalkyl, C₃-C₇ cycloalkyl and 3- to 7-membered heterocycloalkyl are each unsubstituted.

In some embodiments,

R² is selected from C₁-C₆ alkyl, halo, CN, COC₁-C₆ alkyl, CO₂C₁-C₆ alkyl, C₆-C₁₀ aryl, S(O)C₁-C₆ alkyl, 5- to 10-membered heteroaryl, and 3- to 7-membered heterocycloalkyl, wherein the C₁-C₆ alkyl and 3- to 7-membered heterocycloalkyl is optionally substituted with one or more substituents each independently selected from hydroxy and oxo.

In some embodiments, n=1; and

R² is selected from C₁-C₆ alkyl, C₁-C₆ haloalkyl, C₁-C₆ alkoxy, C₁-C₆ haloalkoxy, halo, CN, NO₂, COC₁-C₆ alkyl, CO—C₆-C₁₀ aryl, CO(5- to 10-membered heteroaryl), CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, S(O₂)NR¹¹R¹², S(O)C₁-C₆ alkyl, C₃-C₇ cycloalkyl and 3- to 7-membered heterocycloalkyl,
wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), and OCO(3- to 7-membered heterocycloalkyl);
wherein the 3- to 7-membered heterocycloalkyl, C₆-C₁₀ aryl, 5- to 10-membered heteroaryl of the R² C₁-C₆ alkyl, the R² C₁-C₆ haloalkyl, the R² C₃-C₇ cycloalkyl, or the R² 3- to 7-membered heterocycloalkyl are optionally substituted with one or more substituents independently selected from halo, C₁-C₆ alkyl, and OC₁-C₆ alkyl.

In some embodiments, n=1; and,

R² is selected from C₁-C₆ alkyl, halo, CN, COC₁-C₆ alkyl, CO₂C₁-C₆ alkyl, C₆-C₁₀ aryl, 5- to 10-membered heteroaryl, S(O)C₁-C₆ alkyl, and 3- to 7-membered heterocycloalkyl, wherein the C₁-C₆ alkyl and 3- to 7-membered heterocycloalkyl is optionally substituted with one or more substituents each independently selected from hydroxy and oxo.

In some embodiments, n=1; and

R² is selected from C₁-C₆ alkyl, C₁-C₆ haloalkyl, C₁-C₆ alkoxy, C₁-C₆ haloalkoxy, halo, CN, NO₂, COC₁-C₆ alkyl, CO—C₆-C₁₀ aryl, CO(5- to 10-membered heteroaryl), CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, S(O₂)NR¹¹R¹², S(O)C₁-C₆ alkyl, C₃-C₇ cycloalkyl and 3- to 7-membered heterocycloalkyl, wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), and OCO(3- to 7-membered heterocycloalkyl);
wherein the 3- to 7-membered heterocycloalkyl, C₆-C₁₀ aryl, 5- to 10-membered heteroaryl of the R² C₁-C₆ alkyl, the R² C₁-C₆ haloalkyl, the R² C₃-C₇ cycloalkyl, or the R² 3- to 7-membered heterocycloalkyl are optionally substituted with one or more substituents independently selected from halo, C₁-C₆ alkyl, and OC₁-C₆ alkyl.

In some embodiments, n=1; and,

R² is selected from C₁-C₆ alkyl, halo, CN, COC₁-C₆ alkyl, CO₂C₁-C₆ alkyl, C₆-C₁₀ aryl, 5- to 10-membered heteroaryl, S(O)C₁-C₆ alkyl, and 3- to 7-membered heterocycloalkyl, wherein the C₁-C₆ alkyl and 3- to 7-membered heterocycloalkyl is optionally substituted with one or more substituents each independently selected from hydroxy and oxo.

### Particular Embodiments Wherein n=1

In some embodiments, one of R^{1a} and R^{1b} is hydroxymethyl, the other one of R^{1a} and R^{1b} is hydroxymethyl, and R² is C₁-C₆ alkyl optionally substituted with one or more hydroxyl (e.g., methyl, isopropyl, 2-hydroxy-2-propyl, or 1-hydroxyethyl). In some embodiments, one of R^{1a} and R^{1b} is hydroxymethyl, the other one of R^{1a} and R^{1b} is hydroxy ethyl, and R² is C₁-C₆ alkyl optionally substituted with one or more hydroxyl (e.g., methyl, isopropyl, 2-hydroxy-2-propyl, or 1-hydroxyethyl). In some embodiments, one of R^{1a} and R^{1b} is hydroxymethyl, the other one of R^{1a} and R^{1b} is 2-hydroxy-2-propyl, and R² is C₁-C₆ alkyl optionally substituted with one or more hydroxyl (e.g., methyl, isopropyl, 2-hydroxy-2-propyl, or 1-hydroxyethyl). In some embodiments, one of R^{1a} and R^{1b} is hydroxymethyl, the other one of R^{1a} and R^{1b} is 3-hydroxyl-propyl, and R² is C₁-C₆ alkyl optionally substituted with one or more hydroxyl (e.g., methyl, isopropyl, 2-hydroxy-2-propyl, or 1-hydroxyethyl). In some embodiments, one of R^{1a} and R^{1b} is hydroxymethyl, the other one of R^{1a} and R^{1b} is 1-hydroxy-1-propyl, and R² is C₁-C₆ alkyl optionally substituted with one or more hydroxyl (e.g., methyl, isopropyl, 2-hydroxy-2-propyl, or 1-hydroxyethyl). In some embodiments, one of R^{1a} and R^{1b} is hydroxymethyl, the other one of R^{1a} and R^{1b} is 2-hydroxy-1-propyl, and R² is C₁-C₆ alkyl optionally substituted with one or more hydroxyl (e.g., methyl, isopropyl, 2-hydroxy-2-propyl, or 1-hydroxyethyl). In some embodiments, one of R^{1a} and R^{1b} is hydroxymethyl, the other one of R^{1a} and R^{1b} is 3-hydroxy-1-propyl, and R² is C₁-C₆ alkyl optionally substituted with one or more hydroxyl (e.g., methyl, isopropyl, 2-hydroxy-2-propyl, or 1-hydroxyethyl). In some embodiments, one of R^{1a} and R^{1b} is hydroxymethyl, the other one of R^{1a} and R^{1b} is hydroxybutyl, and R² is C₁-C₆ alkyl optionally substituted with one or more hydroxyl (e.g., methyl, isopropyl, 2-hydroxy-2-propyl, or 1-hydroxyethyl). In some embodiments, one of R^{1a} and R^{1b} is hydroxymethyl, the other one of R^{1a} and R^{1b} is hydroxypentyl, and R² is C₁-C₆ alkyl optionally substituted with one or more hydroxyl (e.g., methyl, isopropyl, 2-hydroxy-2-propyl, or 1-hydroxyethyl). In some embodiments, one of R^{1a} and R^{1b} is hydroxymethyl, the other one of R^{1a} and R^{1b} is hydroxyhexyl, and R² is C₁-C₆ alkyl optionally substituted with one or more hydroxyl (e.g., methyl, isopropyl, 2-hydroxy-2-propyl, or 1-hydroxy ethyl). In some embodiments, one of R^{1a} and R^{1b} is hydroxy ethyl, the other one of R^{1a} and R^{1b} is hydroxymethyl, and R² is C₁-C₆ alkyl optionally substituted with one or more hydroxyl (e.g., methyl, isopropyl, 2-hydroxy-2-propyl, or 1-hydroxyethyl). In some embodiments, one of R^{1a} and R^{1b} is hydroxy ethyl, the other one of R^{1a} and R^{1b} is hydroxy ethyl, and R² is C₁-C₆ alkyl optionally substituted with one or more hydroxyl (e.g., methyl, isopropyl, 2-hydroxy-2-propyl, or 1-hydroxyethyl). In some embodiments, one of R^{1a} and R^{1b} is hydroxy ethyl, the other one of R^{1a} and R^{1b} is 2-hydroxy-2-propyl, and R² is C₁-C₆ alkyl optionally substituted with one or more hydroxyl (e.g., methyl, isopropyl, 2-hydroxy-2-propyl, or 1-hydroxy ethyl). In some embodiments, one of R^{1a} and R^{1b} is hydroxy ethyl, the other one of R^{1a} and R^{1b} is 3-hydroxy-2-propyl, and R² is C₁-C₆ alkyl optionally substituted with one or more hydroxyl (e.g., methyl, isopropyl, 2-hydroxy-2-propyl, or 1-hydroxyethyl). In some embodiments, one of R^{1a} and R^{1b} is hydroxyethyl, the other one of R^{1a} and R^{1b} is 1-hydroxy-1-propyl, and R² is C₁-C₆ alkyl optionally substituted with one or more hydroxyl (e.g., methyl, isopropyl, 2-hydroxy-2-propyl, or 1-hydroxyethyl). In some embodiments, one of R^{1a} and R^{1b} is hydroxyethyl, the other one of R^{1a} and R^{1b} is 2-hydroxy-1-propyl, and R² is C₁-C₆ alkyl optionally substituted with one or more hydroxyl (e.g., methyl, isopropyl, 2-hydroxy-2-propyl, or 1-hydroxyethyl). In some embodiments, one of R^{1a} and R^{1b} is hydroxyethyl, the other one of R^{1a} and R^{1b} is 3-hydroxy-1-propyl, and R² is C₁-C₆ alkyl optionally substituted with one or more hydroxyl (e.g., methyl, isopropyl, 2-hydroxy-2-propyl, or 1-hydroxyethyl). In some embodiments, one of R^{1a} and R^{1b} is hydroxy ethyl, the other one of R^{1a} and R^{1b} is hydroxybutyl, and R² is C₁-C₆ alkyl optionally substituted with one or more hydroxyl (e.g., methyl, isopropyl, 2-hydroxy-2-propyl, or 1-hydroxyethyl). In some embodiments, one of R^{1a} and R^{1b} is hydroxyethyl, the other one of R^{1a} and R^{1b} is hydroxypentyl, and R² is C₁-C₆ alkyl optionally substituted with one or more hydroxyl (e.g., methyl, isopropyl, 2-hydroxy-2-propyl, or 1-hydroxyethyl). In some embodiments, one of R^{1a} and R^{1b} is hydroxyethyl, the other one of R^{1a} and R^{1b} is hydroxyhexyl, and R² is C₁-C₆ alkyl optionally substituted with one or more hydroxyl (e.g., methyl, isopropyl, 2-hydroxy-2-propyl, or 1-hydroxyethyl). In some embodiments, one of R^{1a} and R^{1b} is 2-hydroxy-2-propyl, the other one of R^{1a} and R^{1b} is 2-hydroxy-2-propyl, and R² is C₁-C₆ alkyl optionally substituted with one or more hydroxyl (e.g., methyl, isopropyl, 2-hydroxy-2-propyl, or 1-hydroxyethyl). In some embodiments, one of R^{1a} and R^{1b} is 2-hydroxy-2-propyl, the other one of R^{1a} and R^{1b} is 3-hydroxy-2-propyl, and R² is C₁-C₆ alkyl optionally substituted with one or more hydroxyl (e.g., methyl, isopropyl, 2-hydroxy-2-propyl, or 1-hydroxyethyl). In some embodiments, one of R^{1a} and R^{1b} is 2-hydroxy-2-propyl, the other one of R^{1a} and R^{1b} is 1-hydroxy-1-propyl, and R² is C₁-C₆ alkyl optionally substituted with one or more hydroxyl (e.g., methyl, isopropyl, 2-hydroxy-2-propyl, or 1-hydroxyethyl). In some embodiments, one of R^{1a} and R^{1b} is 2-hydroxy-2-propyl, the other one of R^{1a} and R^{1b} is 2-hydroxy-1-propyl, and R² is C₁-C₆ alkyl optionally substituted with one or more hydroxyl (e.g., methyl, isopropyl, 2-hydroxy-2-propyl, or 1-hydroxyethyl). In some embodiments, one of R^{1a} and R^{1b} is 2-hydroxy-2-propyl, the other one of R^{1a} and R^{1b} is 3-hydroxy-1-propyl, and R² is C₁-C₆ alkyl optionally substituted with one or more hydroxyl (e.g., methyl, isopropyl, 2-hydroxy-2-propyl, or 1-hydroxyethyl). In some embodiments, one of R^{1a} and R^{1b} is 2-hydroxy-2-propyl, the other one of R^{1a} and R^{1b} is hydroxybutyl, and R² is C₁-C₆ alkyl optionally substituted with one or more hydroxyl (e.g., methyl, isopropyl, 2-hydroxy-2-propyl, or 1-hydroxyethyl). In some embodiments, one of R^{1a} and R^{1b} is 2-hydroxy-2-propyl, the other one of R^{1a} and R^{1b} is hydroxypentyl, and R² is C₁-C₆ alkyl optionally substituted with one or more hydroxyl (e.g., methyl, isopropyl, 2-hydroxy-2-propyl, or 1-hydroxyethyl). In some embodiments, one of R^{1a} and R^{1b} is 2-hydroxy-2-propyl, the other one of R^{1a} and R^{1b} is hydroxyhexyl, and R² is C₁-C₆, alkyl optionally substituted with one or more hydroxyl (e.g., methyl, isopropyl, 2-hydroxy-2-propyl, or 1-hydroxyethyl). In some embodiments, one of R^{1a} and R^{1b} is hydroxymethyl, the other one of R^{1a} and R^{1b} is hydroxymethyl, and R² is C₆-C₁₀ aryl (e.g., phenyl). In some embodiments, one of R^{1a} and R^{1b} is hydroxymethyl, the other one of R^{1a} and R^{1b} is hydroxy ethyl, and R² is C₆-C₁₀ aryl (e.g., phenyl). In some embodiments, one of R^{1a} and R^{1b} is hydroxymethyl, the other one of R^{1a} and R^{1b} is 2-hydroxy-2-propyl, and R² is C₆-C₁₀ aryl (e.g., phenyl). In some embodiments, one of R^{1a} and R^{1b} is hydroxymethyl, the other one of R^{1a} and R^{1b} is 3-hydroxyl-propyl, and R² is C₆-C₁₀ aryl (e.g., phenyl). In some embodiments, one of R^{1a} and R^{1b} is hydroxymethyl, the other one of R^{1a} and R^{1b} is 1-hydroxy-1-propyl, and R² is C₆-C₁₀ aryl (e.g., phenyl). In some embodiments, one of R^{1a} and R^{1b} is hydroxymethyl, the other one of R^{1a} and R^{1b} is 2-hydroxy-1-propyl, and R² is C₆-C₁₀ aryl (e.g., phenyl). In some embodiments, one of R^{1a} and R^{1b} is hydroxymethyl, the other one of R^{1a} and R^{1b} is 3-hydroxy-1-propyl, and R² is C₆-C₁₀ aryl (e.g., phenyl). In some embodiments, one of R^{1a} and R^{1b} is hydroxymethyl, the other one of R^{1a} and R^{1b} is hydroxybutyl, and R² is C₆-C₁₀ aryl (e.g., phenyl). In some embodiments, one of R^{1a} and R^{1b} is hydroxymethyl, the other one of R^{1a} and R^{1b} is hydroxypentyl, and R² is C₆-C₁₀ aryl (e.g., phenyl). In some embodiments, one of R^{1a} and R^{1b} is hydroxymethyl, the other one of R^{1a} and R^{1b} is hydroxyhexyl, and R² is C₆-C₁₀ aryl (e.g., phenyl). In some embodiments, one of R^{1a} and R^{1b} is hydroxy ethyl, the other one of R^{1a} and R^{1b} is hydroxy ethyl, and R² is C₆-C₁₀ aryl (e.g., phenyl). In some embodiments, one of R^{1a} and R^{1b} is hydroxy ethyl, the other one of R^{1a} and R^{1b} is 2-hydroxy-2-propyl, and R² is C₆-C₁₀ aryl (e.g., phenyl). In some embodiments, one of R^{1a} and R^{1b} is hydroxyethyl, the other one of R^{1a} and R^{1b} is 3-hydroxy-2-propyl, and R² is C₆-C₁₀ aryl (e.g., phenyl). In some embodiments, one of R^{1a} and R^{1b} is hydroxyethyl, the other one of R^{1a} and R^{1b} is 1-hydroxy-1-propyl, and R² is C₆-C₁₀ aryl (e.g., phenyl). In some embodiments, one of R^{1a} and R^{1b} is hydroxyethyl, the other one of R^{1a} and R^{1b} is 2-hydroxy-1-propyl, and R² is C₆-C₁₀ aryl (e.g., phenyl). In some embodiments, one of R^{1a} and R^{1b} is hydroxyethyl, the other one of R^{1a} and R^{1b} is 3-hydroxy-1-propyl, and R² is C₆-C₁₀ aryl (e.g., phenyl). In some embodiments, one of R^{1a} and R^{1b} is hydroxyethyl, the other one of R^{1a} and R^{1b} is hydroxybutyl, and R² is C₆-C₁₀ aryl (e.g., phenyl). In some embodiments, one of R^{1a} and R^{1b} is hydroxyethyl, the other one of R^{1a} and R^{1b} is hydroxypentyl, and R² is C₆-C₁₀ aryl (e.g., phenyl). In some embodiments, one of R^{1a} and R^{1b} is hydroxyethyl, the other one of R^{1a} and R^{1b} is hydroxyhexyl, and R² is C₆-C₁₀ aryl (e.g., phenyl). In some embodiments, one of R^{1a} and R^{1b} is 2-hydroxy-2-propyl, the other one of R^{1a} and R^{1b} is 2-hydroxy-2-propyl, and R² is C₆-C₁₀ aryl (e.g., phenyl). In some embodiments, one of R^{1a} and R^{1b} is 2-hydroxy-2-propyl, the other one of R^{1a} and R^{1b} is 3-hydroxy-2-propyl, and R² is C₆-C₁₀ aryl (e.g., phenyl). In some embodiments, one of R^{1a} and R^{1b} is 2-hydroxy-2-propyl, the other one of R^{1a} and R^{1b} is 1-hydroxy-1-propyl, and R² is C₆-C₁₀ aryl (e.g., phenyl). In some embodiments, one of R^{1a} and R^{1b} is 2-hydroxy-2-propyl, the other one of R^{1a} and R^{1b} is 2-hydroxy-1-propyl, and R² is C₆-C₁₀ aryl (e.g., phenyl). In some embodiments, one of R^{1a} and R^{1b} is 2-hydroxy-2-propyl, the other one of R^{1a} and R^{1b} is 3-hydroxy-1-propyl, and R² is C₆-C₁₀ aryl (e.g., phenyl). In some embodiments, one of R^{1a} and R^{1b} is 2-hydroxy-2-propyl, the other one of R^{1a} and R^{1b} is hydroxybutyl, and R² is C₆-C₁₀ aryl (e.g., phenyl). In some embodiments, one of R^{1a} and R^{1b} is 2-hydroxy-2-propyl, the other one of R^{1a} and R^{1b} is hydroxypentyl, and R² is C₆-C₁₀ aryl (e.g., phenyl). In some embodiments, one of R^{1a} and R^{1b} is 2-hydroxy-2-propyl, the other one of R^{1a} and R^{1b} is hydroxyhexyl, and R² is C₆-C₁₀ aryl (e.g., phenyl). In some embodiments, one of R^{1a} and R^{1b} is hydroxymethyl, the other one of R^{1a} and R^{1b} is hydroxymethyl, and R² is 5- to 10-membered heteroaryl (e.g., pyridyl or pyrazolyl). In some embodiments, one of R^{1a} and R^{1b} is hydroxymethyl, the other one of R^{1a} and R^{1b} is hydroxy ethyl, and R² is 5- to 10-membered heteroaryl (e.g., pyridyl or pyrazolyl). In some embodiments, one of R^{1a} and R^{1b} is hydroxymethyl, the other one of R^{1a} and R^{1b} is 2-hydroxy-2-propyl, and R² is 5- to 10-membered heteroaryl (e.g., pyridyl or pyrazolyl). In some embodiments, one of R^{1a} and R^{1b} is hydroxymethyl, the other one of R^{1a} and R^{1b} is 3-hydroxy-2-propyl, and R² is 5- to 10-membered heteroaryl (e.g., pyridyl or pyrazolyl). In some embodiments, one of R^{1a} and R^{1b} is hydroxymethyl, the other one of R^{1a} and R^{1b} is 1-hydroxy-1-propyl, and R² is 5- to 10-membered heteroaryl (e.g., pyridyl or pyrazolyl). In some embodiments, one of R^{1a} and R^{1b} is hydroxymethyl, the other one of R^{1a} and R^{1b} is 2-hydroxy-1-propyl, and R² is 5- to 10-membered heteroaryl (e.g., pyridyl or pyrazolyl). In some embodiments, one of R^{1a} and R^{1b} is hydroxymethyl, the other one of R^{1a} and R^{1b} is 3-hydroxy-1-propyl, and R² is 5- to 10-membered heteroaryl (e.g., pyridyl or pyrazolyl). In some embodiments, one of R^{1a} and R^{1b} is hydroxymethyl, the other one of R^{1a} and R^{1b} is hydroxybutyl, and R² is 5- to 10-membered heteroaryl (e.g., pyridyl or pyrazolyl). In some embodiments, one of R^{1a} and R^{1b} is hydroxymethyl, the other one of R^{1a} and R^{1b} is hydroxypentyl, and R² is 5- to 10-membered heteroaryl (e.g., pyridyl or pyrazolyl). In some embodiments, one of R^{1a} and R^{1b} is hydroxymethyl, the other one of R^{1a} and R^{1b} is hydroxyhexyl, and R² is 5- to 10-membered heteroaryl (e.g., pyridyl or pyrazolyl). In some embodiments, one of R^{1a} and R^{1b} is hydroxy ethyl, the other one of R^{1a} and R^{1b} is hydroxy ethyl, and R² is 5- to 10-membered heteroaryl (e.g., pyridyl or pyrazolyl). In some embodiments, one of R^{1a} and R^{1b} is hydroxy ethyl, the other one of R^{1a} and R^{1b} is 2-hydroxy-2-propyl, and R² is 5- to 10-membered heteroaryl (e.g., pyridyl or pyrazolyl). In some embodiments, one of R^{1a} and R^{1b} is hydroxyethyl, the other one of R^{1a} and R^{1b} is 3-hydroxy-2-propyl, and R² is 5- to 10-membered heteroaryl (e.g., pyridyl or pyrazolyl). In some embodiments, one of R^{1a} and R^{1b} is hydroxyethyl, the other one of R^{1a} and R^{1b} is 1-hydroxy-1-propyl, and R² is 5- to 10-membered heteroaryl (e.g., pyridyl or pyrazolyl). In some embodiments, one of R^{1a} and R^{1b} is hydroxyethyl, the other one of R^{1a} and R^{1b} is 2-hydroxy-1-propyl, and R² is 5- to 10-membered heteroaryl (e.g., pyridyl or pyrazolyl). In some embodiments, one of R^{1a} and R^{1b} is hydroxyethyl, the other one of R^{1a} and R^{1b} is 3-hydroxy-1-propyl, and R² is 5- to 10-membered heteroaryl (e.g., pyridyl or pyrazolyl). In some embodiments, one of R^{1a} and R^{1b} is hydroxyethyl, the other one of R^{1a} and R^{1b} is hydroxybutyl, and R² is 5- to 10-membered heteroaryl (e.g., pyridyl or pyrazolyl). In some embodiments, one of R^{1a} and R^{1b} is hydroxyethyl, the other one of R^{1a} and R^{1b} is hydroxypentyl, and R² is 5- to 10-membered heteroaryl (e.g., pyridyl or pyrazolyl). In some embodiments, one of R^{1a} and R^{1b} is hydroxyethyl, the other one of R^{1a} and R^{1b} is hydroxyhexyl, and R² is 5- to 10-membered heteroaryl (e.g., pyridyl or pyrazolyl). In some embodiments, one of R^{1a} and R^{1b} is 2-hydroxy-2-propyl, the other one of R^{1a} and R^{1b} is 2-hydroxy-2-propyl, and R² is 5- to 10-membered heteroaryl (e.g., pyridyl or pyrazolyl). In some embodiments, one of R^{1a} and R^{1b} is 2-hydroxy-2-propyl, the other one of R^{1a} and R^{1b} is 3-hydroxy-2-propyl, and R² is 5- to 10-membered heteroaryl (e.g., pyridyl or pyrazolyl). In some embodiments, one of R^{1a} and R^{1b} is 2-hydroxy-2-propyl, the other one of R^{1a} and R^{1b} is 1-hydroxy-1-propyl, and R² is 5- to 10-membered heteroaryl (e.g., pyridyl or pyrazolyl). In some embodiments, one of R^{1a} and R^{1b} is 2-hydroxy-2-propyl, the other one of R^{1a} and R^{1b} is 2-hydroxy-1-propyl, and R² is 5- to 10-membered heteroaryl (e.g., pyridyl or pyrazolyl). In some embodiments, one of R^{1a} and R^{1b} is 2-hydroxy-2-propyl, the other one of R^{1a} and R^{1b} is 3-hydroxy-1-propyl, and R² is 5- to 10-membered heteroaryl (e.g., pyridyl or pyrazolyl). In some embodiments, one of R^{1a} and R^{1b} is 2-hydroxy-2-propyl, the other one of R^{1a} and R^{1b} is hydroxybutyl, and R² is 5- to 10-membered heteroaryl (e.g., pyridyl or pyrazolyl). In some embodiments, one of R^{1a} and R^{1b} is 2-hydroxy-2-propyl, the other one of R^{1a} and R^{1b} is hydroxypentyl, and R² is 5- to 10-membered heteroaryl (e.g., pyridyl or pyrazolyl). In some embodiments, one of R^{1a} and R^{1b} is 2-hydroxy-2-propyl, the other one of R^{1a} and R^{1b} is hydroxyhexyl, and R² is 5- to 10-membered heteroaryl (e.g., pyridyl or pyrazolyl). In some embodiments, one of R^{1a} and R^{1b} is hydroxymethyl, the other one of R^{1a} and R^{1b} is hydroxymethyl, and R² is SC₁-C₆ alkyl. In some embodiments, one of R^{1a} and R^{1b} is hydroxymethyl, the other one of R^{1a} and R^{1b} is hydroxyethyl, and R² is SC₁-C₆ alkyl. In some embodiments, one of R^{1a} and R^{1b} is hydroxymethyl, the other one of R^{1a} and R^{1b} is 2-hydroxy-2-propyl, and R² is SC₁-C₆ alkyl. In some embodiments, one of R^{1a} and R^{1b} is hydroxymethyl, the other one of R^{1a} and R^{1b} is 3-hydroxy-2-propyl, and R² is SC₁-C₆ alkyl. In some embodiments, one of R^{1a} and R^{1b} is hydroxymethyl, the other one of R^{1a} and R^{1b} is 1-hydroxy-1-propyl, and R² is SC₁-C₆ alkyl. In some embodiments, one of R^{1a} and R^{1b} is hydroxymethyl, the other one of R^{1a} and R^{1b} is 2-hydroxy-1-propyl, and R² is SC₁-C₆ alkyl. In some embodiments, one of R^{1a} and R^{1b} is hydroxymethyl, the other one of R^{1a} and R^{1b} is 3-hydroxy-1-propyl, and R² is SC₁-C₆ alkyl. In some embodiments, one of R^{1a} and R^{1b} is hydroxymethyl, the other one of R^{1a} and R^{1b} is hydroxybutyl, and R² is SC₁-C₆ alkyl. In some embodiments, one of R^{1a} and R^{1b} is hydroxymethyl, the other one of R^{1a} and R^{1b} is hydroxypentyl, and R² is SC₁-C₆ alkyl. In some embodiments, one of R^{1a} and R^{1b} is hydroxymethyl, the other one of R^{1a} and R^{1b} is hydroxyhexyl, and R² is SC₁-C₆ alkyl. In some embodiments, one of R^{1a} and R^{1b} is hydroxyethyl, the other one of R^{1a} and R^{1b} is hydroxyethyl, and R² is SC₁-C₆ alkyl. In some embodiments, one of R^{1a} and R^{1b} is hydroxyethyl, the other one of R^{1a} and R^{1b} is 2-hydroxy-2-propyl, and R² is SC₁-C₆ alkyl. In some embodiments, one of R^{1a} and R^{1b} is hydroxyethyl, the other one of R^{1a} and R^{1b} is 3-hydroxy-2-propyl, and R² is SC₁-C₆ alkyl. In some embodiments, one of R^{1a} and R^{1b} is hydroxyethyl, the other one of R^{1a} and R^{1b} is 1-hydroxy-1-propyl, and R² is SC₁-C₆ alkyl. In some embodiments, one of R^{1a} and R^{1b} is hydroxyethyl, the other one of R^{1a} and R^{1b} is 2-hydroxy-1-propyl, and R² is SC₁-C₆ alkyl. In some embodiments, one of R^{1a} and R^{1b} is hydroxyethyl, the other one of R^{1a} and R^{1b} is 3-hydroxy-1-propyl, and R² is SC₁-C₆ alkyl. In some embodiments, one of R^{1a} and R^{1b} is hydroxyethyl, the other one of R^{1a} and R^{1b} is hydroxybutyl, and R² is SC₁-C₆ alkyl. In some embodiments, one of R^{1a} and R^{1b} is hydroxyethyl, the other one of R^{1a} and R^{1b} is hydroxypentyl, and R² is SC₁-C₆ alkyl. In some embodiments, one of R^{1a} and R^{1b} is hydroxyethyl, the other one of R^{1a} and R^{1b} is hydroxyhexyl, and R² is SC₁-C₆ alkyl. In some embodiments, one of R^{1a} and R^{1b} is 2-hydroxy-2-propyl, the other one of R^{1a} and R^{1b} is 2-hydroxy-2-propyl, and R² is SC₁-C₆ alkyl. In some embodiments, one of R^{1a} and R^{1b} is 2-hydroxy-2-propyl, the other one of R^{1a} and R^{1b} is 3-hydroxy-2-propyl, and R² is SC₁-C₆ alkyl. In some embodiments, one of R^{1a} and R^{1b} is 2-hydroxy-2-propyl, the other one of R^{1a} and R^{1b} is 1-hydroxy-1-propyl, and R² is SC₁-C₆ alkyl. In some embodiments, one of R^{1a} and R^{1b} is 2-hydroxy-2-propyl, the other one of R^{1a} and R^{1b} is 2-hydroxy-1-propyl, and R² is SC₁-C₆ alkyl. In some embodiments, one of R^{1a} and R^{1b} is 2-hydroxy-2-propyl, the other one of R^{1a} and R^{1b} is 3-hydroxy-1-propyl, and R² is SC₁-C₆ alkyl. In some embodiments, one of R^{1a} and R^{1b} is 2-hydroxy-2-propyl, the other one of R^{1a} and R^{1b} is hydroxybutyl, and R² is SC₁-C₆ alkyl. In some embodiments, one of R^{1a} and R^{1b} is 2-hydroxy-2-propyl, the other one of R^{1a} and R^{1b} is hydroxypentyl, and R² is SC₁-C₆ alkyl. In some embodiments, one of R^{1a} and R^{1b} is 2-hydroxy-2-propyl, the other one of R^{1a} and R^{1b} is hydroxyhexyl, and R² is SC₁-C₆ alkyl. In some embodiments, one of R^{1a} and R^{1b} is hydroxymethyl, the other one of R^{1a} and R^{1b} is hydroxymethyl, and R² is S(O₂)C₁-C₆ alkyl (e.g., S(O₂)CH₃). In some embodiments, one of R^{1a} and R^{1b} is hydroxymethyl, the other one of R^{1a} and R^{1b} is hydroxyethyl, and R² is S(Cb)C₁-C₆ alkyl (e.g., S(O₂)CH₃). In some embodiments, one of R^{1a} and R^{1b} is hydroxymethyl, the other one of R^{1a} and R^{1b} is 2-hydroxy-2-propyl, and R² is S(O₂)C₁-C₆ alkyl (e.g., S(O₂)CH₃). In some embodiments, one of R^{1a} and R^{1b} is hydroxymethyl, the other one of R^{1a} and R^{1b} is 3-hydroxyl-propyl, and R² is S(O₂)C₁-C₆ alkyl (e.g., S(O₂)CH₃). In some embodiments, one of R^{1a} and R^{1b} is hydroxymethyl, the other one of R^{1a} and R^{1b} is 1-hydroxy-1-propyl, and R² is S(O₂)C₁-C₆ alkyl (e.g., S(O₂)CH₃). In some embodiments, one of R^{1a} and R^{1b} is hydroxymethyl, the other one of R^{1a} and R^{1b} is 2-hydroxy-1-propyl, and R² is S(O₂)C₁-C₆ alkyl (e.g., S(O₂)CH₃). In some embodiments, one of R^{1a} and R^{1b} is hydroxymethyl, the other one of R^{1a} and R^{1b} is 3-hydroxy-1-propyl, and R² is S(O₂)C₁-C₆ alkyl (e.g., S(O₂)CH₃). In some embodiments, one of R^{1a} and R^{1b} is hydroxymethyl, the other one of R^{1a} and R^{1b} is hydroxybutyl, and R² is S(O₂)C₁-C₆ alkyl (e.g., S(O₂)CH₃). In some embodiments, one of R^{1a} and R^{1b} is hydroxymethyl, the other one of R^{1a} and R^{1b} is hydroxypentyl, and R² is S(O₂)C₁-C₆ alkyl (e.g., S(O₂)CH₃). In some embodiments, one of R^{1a} and R^{1b} is hydroxymethyl, the other one of R^{1a} and R^{1b} is hydroxyhexyl, and R² is S(O₂)C₁-C₆ alkyl (e.g., S(O₂)CH₃). In some embodiments, one of R^{1a} and R^{1b} is hydroxyethyl, the other one of R^{1a} and R^{1b} is hydroxyethyl, and R² is S(O₂)C₁-C₆ alkyl (e.g., S(O₂)CH₃). In some embodiments, one of R^{1a} and R^{1b} is hydroxyethyl, the other one of R^{1a} and R^{1b} is 2-hydroxy-2-propyl, and R² is S(O₂)C₁-C₆ alkyl (e.g., S(O₂)CH₃). In some embodiments, one of R^{1a} and R^{1b} is hydroxyethyl, the other one of R^{1a} and R^{1b} is 3-hydroxy-2-propyl, and R² is S(O₂)C₁-C₆ alkyl (e.g., S(O₂)CH₃). In some embodiments, one of R^{1a} and R^{1b} is hydroxyethyl, the other one of R^{1a} and R^{1b} is 1-hydroxy-1-propyl, and R² is S(O₂)C₁-C₆ alkyl (e.g., S(O₂)CH₃). In some embodiments, one of R^{1a} and R^{1b} is hydroxyethyl, the other one of R^{1a} and R^{1b} is 2-hydroxy-1-propyl, and R² is S(O₂)C₁-C₆ alkyl (e.g., S(O₂)CH₃). In some embodiments, one of R^{1a} and R^{1b} is hydroxyethyl, the other one of R^{1a} and R^{1b} is 3-hydroxy-1-propyl, and R² is S(O₂)C₁-C₆ alkyl (e.g., S(O₂)CH₃). In some embodiments, one of R^{1a} and R^{1b} is hydroxyethyl, the other one of R^{1a} and R^{1b} is hydroxybutyl, and R² is S(O₂)C₁-C₆ alkyl (e.g., S(O₂)CH₃). In some embodiments, one of R^{1a} and R^{1b} is hydroxyethyl, the other one of R^{1a} and R^{1b} is hydroxypentyl, and R² is S(O₂)C₁-C₆ alkyl (e.g., S(O₂)CH₃). In some embodiments, one of R^{1a} and R^{1b} is hydroxyethyl, the other one of R^{1a} and R^{1b} is hydroxyhexyl, and R² is S(O₂)C₁-C₆ alkyl (e.g., S(O₂)CH₃). In some embodiments, one of R^{1a} and R^{1b} is 2-hydroxy-2-propyl, the other one of R^{1a} and R^{1b} is 2-hydroxy-2-propyl, and R² is S(O₂)C₁-C₆ alkyl (e.g., S(O₂)CH₃). In some embodiments, one of R^{1a} and R^{1b} is 2-hydroxy-2-propyl, the other one of R^{1a} and R^{1b} is 3-hydroxy-2-propyl, and R² is S(O₂)C₁-C₆ alkyl (e.g., S(O₂)CH₃). In some embodiments, one of R^{1a} and R^{1b} is 2-hydroxy-2-propyl, the other one of R^{1a} and R^{1b} is 1-hydroxy-1-propyl, and R² is S(O₂)C₁-C₆ alkyl (e.g., S(O₂)CH₃). In some embodiments, one of R^{1a} and R^{1b} is 2-hydroxy-2-propyl, the other one of R^{1a} and R^{1b} is 2-hydroxy-1-propyl, and R² is S(O₂)C₁-C₆ alkyl (e.g., S(O₂)CH₃). In some embodiments, one of R^{1a} and R^{1b} is 2-hydroxy-2-propyl, the other one of R^{1a} and R^{1b} is 3-hydroxy-1-propyl, and R² is S(O₂)C₁-C₆ alkyl (e.g., S(O₂)CH₃). In some embodiments, one of R^{1a} and R^{1b} is 2-hydroxy-2-propyl, the other one of R^{1a} and R^{1b} is hydroxybutyl, and R² is S(O₂)C₁-C₆ alkyl (e.g., S(O₂)CH₃). In some embodiments, one of R^{1a} and R^{1b} is 2-hydroxy-2-propyl, the other one of R^{1a} and R^{1b} is hydroxypentyl, and R² is S(O₂)C₁-C₆ alkyl (e.g., S(O₂)CH₃). In some embodiments, one of R^{1a} and R^{1b} is 2-hydroxy-2-propyl, the other one of R^{1a} and R^{1b} is hydroxyhexyl, and R² is S(O₂)C₁-C₆ alkyl (e.g., S(O₂)CH₃). In some embodiments, one of R^{1a} and R^{1b} is hydroxymethyl, the other one of R^{1a} and R^{1b} is hydroxymethyl, and R² is halo (e.g., fluoro or chloro). In some embodiments, one of R^{1a} and R^{1b} is hydroxymethyl, the other one of R^{1a} and R^{1b} is hydroxyethyl, and R² is halo (e.g., fluoro or chloro). In some embodiments, one of R^{1a} and R^{1b} is hydroxymethyl, the other one of R^{1a} and R^{1b} is 2-hydroxy-2-propyl, and R² is halo (e.g., fluoro or chloro). In some embodiments, one of R^{1a} and R^{1b} is hydroxymethyl, the other one of R^{1a} and R^{1b} is 3-hydroxy-2-propyl, and R² is halo (e.g., fluoro or chloro). In some embodiments, one of R^{1a} and R^{1b} is hydroxymethyl, the other one of R^{1a} and R^{1b} is 1-hydroxy-1-propyl, and R² is halo (e.g., fluoro or chloro). In some embodiments, one of R^{1a} and R^{1b} is hydroxymethyl, the other one of R^{1a} and R^{1b} is 2-hydroxy-1-propyl, and R² is halo (e.g., fluoro or chloro). In some embodiments, one of R^{1a} and R^{1b} is hydroxymethyl, the other one of R^{1a} and R^{1b} is 3-hydroxy-1-propyl, and R² is halo (e.g., fluoro or chloro). In some embodiments, one of R^{1a} and R^{1b} is hydroxymethyl, the other one of R^{1a} and R^{1b} is hydroxybutyl, and R² is halo (e.g., fluoro or chloro). In some embodiments, one of R^{1a} and R^{1b} is hydroxymethyl, the other one of R^{1a} and R^{1b} is hydroxypentyl, and R² is halo (e.g., fluoro or chloro). In some embodiments, one of R^{1a} and R^{1b} is hydroxymethyl, the other one of R^{1a} and R^{1b} is hydroxyhexyl, and R² is halo (e.g., fluoro or chloro). In some embodiments, one of R^{1a} and R^{1b} is hydroxyethyl, the other one of R^{1a} and R^{1b} is hydroxyethyl, and R² is halo (e.g., fluoro or chloro). In some embodiments, one of R^{1a} and R^{1b} is hydroxyethyl, the other one of R^{1a} and R^{1b} is 2-hydroxy-2-propyl, and R² is halo (e.g., fluoro or chloro). In some embodiments, one of R^{1a} and R^{1b} is hydroxyethyl, the other one of R^{1a} and R^{1b} is 3-hydroxy-2-propyl, and R² is halo (e.g., fluoro or chloro). In some embodiments, one of R^{1a} and R^{1b} is hydroxyethyl, the other one of R^{1a} and R^{1b} is 1-hydroxy-1-propyl, and R² is halo (e.g., fluoro or chloro). In some embodiments, one of R^{1a} and R^{1b} is hydroxyethyl, the other one of R^{1a} and R^{1b} is 2-hydroxy-1-propyl, and R² is halo (e.g., fluoro or chloro). In some embodiments, one of R^{1a} and R^{1b} is hydroxyethyl, the other one of R^{1a} and R^{1b} is 3-hydroxy-1-propyl, and R² is halo (e.g., fluoro or chloro). In some embodiments, one of R^{1a} and R^{1b} is hydroxyethyl, the other one of R^{1a} and R^{1b} is hydroxybutyl, and R² is halo (e.g., fluoro or chloro). In some embodiments, one of R^{1a} and R^{1b} is hydroxyethyl, the other one of R^{1a} and R^{1b} is hydroxypentyl, and R² is halo (e.g., fluoro or chloro). In some embodiments, one of R^{1a} and R^{1b} is hydroxyethyl, the other one of R^{1a} and R^{1b} is hydroxyhexyl, and R² is halo (e.g., fluoro or chloro). In some embodiments, one of R^{1a} and R^{1b} is 2-hydroxy-2-propyl, the other one of R^{1a} and R^{1b} is 2-hydroxy-2-propyl, and R² is halo (e.g., fluoro or chloro). In some embodiments, one of R^{1a} and R^{1b} is 2-hydroxy-2-propyl, the other one of R^{1a} and R^{1b} is 3-hydroxy-2-propyl, and R² is halo (e.g., fluoro or chloro). In some embodiments, one of R^{1a} and R^{1b} is 2-hydroxy-2-propyl, the other one of R^{1a} and R^{1b} is 1-hydroxy-1-propyl, and R² is halo (e.g., fluoro or chloro). In some embodiments, one of R^{1a} and R^{1b} is 2-hydroxy-2-propyl, the other one of R^{1a} and R^{1b} is 2-hydroxy-1-propyl, and R² is halo (e.g., fluoro or chloro). In some embodiments, one of R^{1a} and R^{1b} is 2-hydroxy-2-propyl, the other one of R^{1a} and R^{1b} is 3-hydroxy-1-propyl, and R² is halo (e.g., fluoro or chloro). In some embodiments, one of R^{1a} and R^{1b} is 2-hydroxy-2-propyl, the other one of R^{1a} and R^{1b} is hydroxybutyl, and R² is halo (e.g., fluoro or chloro). In some embodiments, one of R^{1a} and R^{1b} is 2-hydroxy-2-propyl, the other one of R^{1a} and R^{1b} is hydroxypentyl, and R² is halo (e.g., fluoro or chloro). In some embodiments, one of R^{1a} and R^{1b} is 2-hydroxy-2-propyl, the other one of R^{1a} and R^{1b} is hydroxyhexyl, and R² is halo (e.g., fluoro or chloro). In some embodiments, one of R^{1a} and R^{1b} is hydroxymethyl, the other one of R^{1a} and R^{1b} is hydroxymethyl, and R² is C₃-C₇ cycloalkyl optionally substituted with one or more hydroxy (e.g. 1-hydroxy-1-cyclopropyl, 1-hydroxy-1-cyclobutyl, 1-hydroxy-1-cyclopentyl, or 1-hydroxy-1-cyclohexyl). In some embodiments, one of R^{1a} and R^{1b} is hydroxymethyl, the other one of R^{1a} and R^{1b} is hydroxy ethyl, and R² is C₃-C₇ cycloalkyl optionally substituted with one or more hydroxy (e.g. 1-hydroxy-1-cyclopropyl, 1-hydroxy-1-cyclobutyl, 1-hydroxy-1-cyclopentyl, or 1-hydroxy-1-cyclohexyl). In some embodiments, one of R^{1a} and R^{1b} is hydroxymethyl, the other one of R^{1a} and R^{1b} is 2-hydroxy-2-propyl, and R² is C₃-C₇ cycloalkyl optionally substituted with one or more hydroxy (e.g. 1-hydroxy-1-cyclopropyl, 1-hydroxy-1-cyclobutyl, 1-hydroxy-1-cyclopentyl, or 1-hydroxy-1-cyclohexyl). In some embodiments, one of R^{1a} and R^{1b} is hydroxymethyl, the other one of R^{1a} and R^{1b} is 3-hydroxy-2-propyl, and R² is C₃-C₇ cycloalkyl optionally substituted with one or more hydroxy (e.g. 1-hydroxy-1-cyclopropyl, 1-hydroxy-1-cyclobutyl, 1-hydroxy-1-cyclopentyl, or 1-hydroxy-1-cyclohexyl). In some embodiments, one of R^{1a} and R^{1b} is hydroxymethyl, the other one of R^{1a} and R^{1b} is 1-hydroxy-1-propyl, and R² is C₃-C₇ cycloalkyl optionally substituted with one or more hydroxy (e.g. 1-hydroxy-1-cyclopropyl, 1-hydroxy-1-cyclobutyl, 1-hydroxy-1-cyclopentyl, or 1-hydroxy-1-cyclohexyl). In some embodiments, one of R^{1a} and R^{1b} is hydroxymethyl, the other one of R^{1a} and R^{1b} is 2-hydroxy-1-propyl, and R² is C₃-C₇ cycloalkyl optionally substituted with one or more hydroxy (e.g. 1-hydroxy-1-cyclopropyl, 1-hydroxy-1-cyclobutyl, 1-hydroxy-1-cyclopentyl, or 1-hydroxy-1-cyclohexyl). In some embodiments, one of R^{1a} and R^{1b} is hydroxymethyl, the other one of R^{1a} and R^{1b} is 3-hydroxy-1-propyl, and R² is C₃-C₇ cycloalkyl optionally substituted with one or more hydroxy (e.g. 1-hydroxy-1-cyclopropyl, 1-hydroxy-1-cyclobutyl, 1-hydroxy-1-cyclopentyl, or 1-hydroxy-1-cyclohexyl). In some embodiments, one of R^{1a} and R^{1b} is hydroxymethyl, the other one of R^{1a} and R^{1b} is hydroxybutyl, and R² is C₃-C₇ cycloalkyl optionally substituted with one or more hydroxy (e.g. 1-hydroxy-1-cyclopropyl, 1-hydroxy-1-cyclobutyl, 1-hydroxy-1-cyclopentyl, or 1-hydroxy-1-cyclohexyl). In some embodiments, one of R^{1a} and R^{1b} is hydroxymethyl, the other one of R^{1a} and R^{1b} is hydroxypentyl, and R² is C₃-C₇ cycloalkyl optionally substituted with one or more hydroxy (e.g. 1-hydroxy-1-cyclopropyl, 1-hydroxy-1-cyclobutyl, 1-hydroxy-1-cyclopentyl, or 1-hydroxy-1-cyclohexyl). In some embodiments, one of R^{1a} and R^{1b} is hydroxymethyl, the other one of R^{1a} and R^{1b} is hydroxyhexyl, and R² is C₃-C₇ cycloalkyl optionally substituted with one or more hydroxy (e.g. 1-hydroxy-1-cyclopropyl, 1-hydroxy-1-cyclobutyl, 1-hydroxy-1-cyclopentyl, or 1-hydroxy-1-cyclohexyl). In some embodiments, one of R^{1a} and R^{1b} is hydroxyethyl, the other one of R^{1a} and R^{1b} is hydroxyethyl, and R² is C₃-C₇ cycloalkyl optionally substituted with one or more hydroxy (e.g. 1-hydroxy-1-cyclopropyl, 1-hydroxy-1-cyclobutyl, 1-hydroxy-1-cyclopentyl, or 1-hydroxy-1-cyclohexyl). In some embodiments, one of R^{1a} and R^{1b} is hydroxyethyl, the other one of R^{1a} and R^{1b} is 2-hydroxy-2-propyl, and R² is C₃-C₇ cycloalkyl optionally substituted with one or more hydroxy (e.g. 1-hydroxy-1-cyclopropyl, 1-hydroxy-1-cyclobutyl, 1-hydroxy-1-cyclopentyl, or 1-hydroxy-1-cyclohexyl). In some embodiments, one of R^{1a} and R^{1b} is hydroxyethyl, the other one of R^{1a} and R^{1b} is 3-hydroxy-2-propyl, and R² is C₃-C₇ cycloalkyl optionally substituted with one or more hydroxy (e.g. 1-hydroxy-1-cyclopropyl, 1-hydroxy-1-cyclobutyl, 1-hydroxy-1-cyclopentyl, or 1-hydroxy-1-cyclohexyl). In some embodiments, one of R^{1a} and R^{1b} is hydroxyethyl, the other one of R^{1a} and R^{1b} is 1-hydroxy-1-propyl, and R² is C₃-C₇ cycloalkyl optionally substituted with one or more hydroxy (e.g. 1-hydroxy-1-cyclopropyl, 1-hydroxy-1-cyclobutyl, 1-hydroxy-1-cyclopentyl, or 1-hydroxy-1-cyclohexyl). In some embodiments, one of R^{1a} and R^{1b} is hydroxyethyl, the other one of R^{1a} and R^{1b} is 2-hydroxy-1-propyl, and R² is C₃-C₇ cycloalkyl optionally substituted with one or more hydroxy (e.g. 1-hydroxy-1-cyclopropyl, 1-hydroxy-1-cyclobutyl, 1-hydroxy-1-cyclopentyl, or 1-hydroxy-1-cyclohexyl). In some embodiments, one of R^{1a} and R^{1b} is hydroxyethyl, the other one of R^{1a} and R^{1b} is 3-hydroxy-1-propyl, and R² is C₃-C₇ cycloalkyl optionally substituted with one or more hydroxy (e.g. 1-hydroxy-1-cyclopropyl, 1-hydroxy-1-cyclobutyl, 1-hydroxy-1-cyclopentyl, or 1-hydroxy-1-cyclohexyl). In some embodiments, one of R^{1a} and R^{1b} is hydroxyethyl, the other one of R^{1a} and R^{1b} is hydroxybutyl, and R² is C₃-C₇ cycloalkyl optionally substituted with one or more hydroxy (e.g. 1-hydroxy-1-cyclopropyl, 1-hydroxy-1-cyclobutyl, 1-hydroxy-1-cyclopentyl, or 1-hydroxy-1-cyclohexyl). In some embodiments, one of R^{1a} and R^{1b} is hydroxyethyl, the other one of R^{1a} and R^{1b} is hydroxypentyl, and R² is C₃-C₇ cycloalkyl optionally substituted with one or more hydroxy (e.g. 1-hydroxy-1-cyclopropyl, 1-hydroxy-1-cyclobutyl, 1-hydroxy-1-cyclopentyl, or 1-hydroxy-1-cyclohexyl). In some embodiments, one of R^{1a} and R^{1b} is hydroxy ethyl, the other one of R^{1a} and R^{1b} is hydroxyhexyl, and R² is C₃-C₇ cycloalkyl optionally substituted with one or more hydroxy (e.g. 1-hydroxy-1-cyclopropyl, 1-hydroxy-1-cyclobutyl, 1-hydroxy-1-cyclopentyl, or 1-hydroxy-1-cyclohexyl). In some embodiments, one of R^{1a} and R^{1b} is 2-hydroxy-2-propyl, the other one of R^{1a} and R^{1b} is 2-hydroxy-2-propyl, and R² is C₃-C₇ cycloalkyl optionally substituted with one or more hydroxy (e.g. 1-hydroxy-1-cyclopropyl, 1-hydroxy-1-cyclobutyl, 1-hydroxy-1-cyclopentyl, or 1-hydroxy-1-cyclohexyl). In some embodiments, one of R^{1a} and R^{1b} is 2-hydroxy-2-propyl, the other one of R^{1a} and R^{1b} is 3-hydroxy-2-propyl, and R² is C₃-C₇ cycloalkyl optionally substituted with one or more hydroxy (e.g. 1-hydroxy-1-cyclopropyl, 1-hydroxy-1-cyclobutyl, 1-hydroxy-1-cyclopentyl, or 1-hydroxy-1-cyclohexyl). In some embodiments, one of R^{1a} and R^{1b} is 2-hydroxy-2-propyl, the other one of R^{1a} and R^{1b} is 1-hydroxy-1-propyl, and R² is C₃-C₇ cycloalkyl optionally substituted with one or more hydroxy (e.g. 1-hydroxy-1-cyclopropyl, 1-hydroxy-1-cyclobutyl, 1-hydroxy-1-cyclopentyl, or 1-hydroxy-1-cyclohexyl). In some embodiments, one of R^{1a} and R^{1b} is 2-hydroxy-2-propyl, the other one of R^{1a} and R^{1b} is 2-hydroxy-1-propyl, and R² is C₃-C₇ cycloalkyl optionally substituted with one or more hydroxy (e.g. 1-hydroxy-1-cyclopropyl, 1-hydroxy-1-cyclobutyl, 1-hydroxy-1-cyclopentyl, or 1-hydroxy-1-cyclohexyl). In some embodiments, one of R^{1a} and R^{1b} is 2-hydroxy-2-propyl, the other one of R^{1a} and R^{1b} is 3-hydroxy-1-propyl, and R² is C₃-C₇ cycloalkyl optionally substituted with one or more hydroxy (e.g. 1-hydroxy-1-cyclopropyl, 1-hydroxy-1-cyclobutyl, 1-hydroxy-1-cyclopentyl, or 1-hydroxy-1-cyclohexyl). In some embodiments, one of R^{1a} and R^{1b} is 2-hydroxy-2-propyl, the other one of R^{1a} and R^{1b} is hydroxybutyl, and R² is C₃-C₇ cycloalkyl optionally substituted with one or more hydroxy (e.g. 1-hydroxy-1-cyclopropyl, 1-hydroxy-1-cyclobutyl, 1-hydroxy-1-cyclopentyl, or 1-hydroxy-1-cyclohexyl). In some embodiments, one of R^{1a} and R^{1b} is 2-hydroxy-2-propyl, the other one of R^{1a} and R^{1b} is hydroxypentyl, and R² is C₃-C₇ cycloalkyl optionally substituted with one or more hydroxy (e.g. 1-hydroxy-1-cyclopropyl, 1-hydroxy-1-cyclobutyl, 1-hydroxy-1-cyclopentyl, or 1-hydroxy-1-cyclohexyl). In some embodiments, one of R^{1a} and R^{1b} is 2-hydroxy-2-propyl, the other one of R^{1a} and R^{1b} is hydroxyhexyl, and R² is C₃-C₇ cycloalkyl optionally substituted with one or more hydroxy (e.g. 1-hydroxy-1-cyclopropyl, 1-hydroxy-1-cyclobutyl, 1-hydroxy-1-cyclopentyl, or 1-hydroxy-1-cyclohexyl). In some embodiments, one of R^{1a} and R^{1b} is hydroxymethyl, the other one of R^{1a} and R^{1b} is hydroxymethyl, and R² is 3- to 7-membered heterocycloalkyl optionally substituted with one or more hydroxy (e.g., morpholinyl or 1,3-dioxolan-2-yl). In some embodiments, one of R^{1a} and R^{1b} is hydroxymethyl, the other one of R^{1a} and R^{1b} is hydroxyethyl, and R² is 3- to 7-membered heterocycloalkyl optionally substituted with one or more hydroxy (e.g., morpholinyl or 1,3-dioxolan-2-yl). In some embodiments, one of R^{1a} and R^{1b} is hydroxymethyl, the other one of R^{1a} and R^{1b} is 2-hydroxy-2-propyl, and R² is 3- to 7-membered heterocycloalkyl optionally substituted with one or more hydroxy (e.g., morpholinyl or 1,3-dioxolan-2-yl). In some embodiments, one of R^{1a} and R^{1b} is hydroxymethyl, the other one of R^{1a} and R^{1b} is 3-hydroxy-2-propyl, and R² is 3- to 7-membered heterocycloalkyl optionally substituted with one or more hydroxy (e.g., morpholinyl or 1,3-dioxolan-2-yl). In some embodiments, one of R^{1a} and R^{1b} is hydroxymethyl, the other one of R^{1a} and R^{1b} is 1-hydroxy-1-propyl, and R² is 3- to 7-membered heterocycloalkyl optionally substituted with one or more hydroxy (e.g., morpholinyl or 1,3-dioxolan-2-yl). In some embodiments, one of R^{1a} and R^{1b} is hydroxymethyl, the other one of R^{1a} and R^{1b} is 2-hydroxy-1-propyl, and R² is 3- to 7-membered heterocycloalkyl optionally substituted with one or more hydroxy (e.g., morpholinyl or 1,3-dioxolan-2-yl). In some embodiments, one of R^{1a} and R^{1b} is hydroxymethyl, the other one of R^{1a} and R^{1b} is 3-hydroxy-1-propyl, and R² is 3- to 7-membered heterocycloalkyl optionally substituted with one or more hydroxy (e.g., morpholinyl or 1,3-dioxolan-2-yl). In some embodiments, one of R^{1a} and R^{1b} is hydroxymethyl, the other one of R^{1a} and R^{1b} is hydroxybutyl, and R² is 3- to 7-membered heterocycloalkyl optionally substituted with one or more hydroxy (e.g., morpholinyl or 1,3-dioxolan-2-yl). In some embodiments, one of R^{1a} and R^{1b} is hydroxymethyl, the other one of R^{1a} and R^{1b} is hydroxypentyl, and R² is 3- to 7-membered heterocycloalkyl optionally substituted with one or more hydroxy (e.g., morpholinyl or 1,3-dioxolan-2-yl). In some embodiments, one of R^{1a} and R^{1b} is hydroxymethyl, the other one of R^{1a} and R^{1b} is hydroxyhexyl, and R² is 3- to 7-membered heterocycloalkyl optionally substituted with one or more hydroxy (e.g., morpholinyl or 1,3-dioxolan-2-yl). In some embodiments, one of R^{1a} and R^{1b} is hydroxyethyl, the other one of R^{1a} and R^{1b} is hydroxyethyl, and R² is 3- to 7-membered heterocycloalkyl optionally substituted with one or more hydroxy (e.g., morpholinyl or 1,3-dioxolan-2-yl). In some embodiments, one of R^{1a} and R^{1b} is hydroxyethyl, the other one of R^{1a} and R^{1b} is 2-hydroxy-2-propyl, and R² is 3- to 7-membered heterocycloalkyl optionally substituted with one or more hydroxy (e.g., morpholinyl or 1,3-dioxolan-2-yl). In some embodiments, one of R^{1a} and R^{1b} is hydroxyethyl, the other one of R^{1a} and R^{1b} is 3-hydroxy-2-propyl, and R² is 3- to 7-membered heterocycloalkyl optionally substituted with one or more hydroxy (e.g., morpholinyl or 1,3-dioxolan-2-yl). In some embodiments, one of R^{1a} and R^{1b} is hydroxyethyl, the other one of R^{1a} and R^{1b} is 1-hydroxy-1-propyl, and R² is 3- to 7-membered heterocycloalkyl optionally substituted with one or more hydroxy (e.g., morpholinyl or 1,3-dioxolan-2-yl). In some embodiments, one of R^{1a} and R^{1b} is hydroxyethyl, the other one of R^{1a} and R^{1b} is 2-hydroxy-1-propyl, and R² is 3- to 7-membered heterocycloalkyl optionally substituted with one or more hydroxy (e.g., morpholinyl or 1,3-dioxolan-2-yl). In some embodiments, one of R^{1a} and R^{1b} is hydroxyethyl, the other one of R^{1a} and R^{1b} is 3-hydroxy-1-propyl, and R² is 3- to 7-membered heterocycloalkyl optionally substituted with one or more hydroxy (e.g., morpholinyl or 1,3-dioxolan-2-yl). In some embodiments, one of R^{1a} and R^{1b} is hydroxyethyl, the other one of R^{1a} and R^{1b} is hydroxybutyl, and R² is 3- to 7-membered heterocycloalkyl optionally substituted with one or more hydroxy (e.g., morpholinyl or 1,3-dioxolan-2-yl). In some embodiments, one of R^{1a} and R^{1b} is hydroxyethyl, the other one of R^{1a} and R^{1b} is hydroxypentyl, and R² is 3- to 7-membered heterocycloalkyl optionally substituted with one or more hydroxy (e.g., morpholinyl or 1,3-dioxolan-2-yl). In some embodiments, one of R^{1a} and R^{1b} is hydroxyethyl, the other one of R^{1a} and R^{1b} is hydroxyhexyl, and R² is 3- to 7-membered heterocycloalkyl optionally substituted with one or more hydroxy (e.g., morpholinyl or 1,3-dioxolan-2-yl). In some embodiments, one of R^{1a} and R^{1b} is 2-hydroxy-2-propyl, the other one of R^{1a} and R^{1b} is 2-hydroxy-2-propyl, and R² is 3- to 7-membered heterocycloalkyl optionally substituted with one or more hydroxy (e.g., morpholinyl or 1,3-dioxolan-2-yl). In some embodiments, one of R^{1a} and R^{1b} is 2-hydroxy-2-propyl, the other one of R^{1a} and R^{1b} is 3-hydroxy-2-propyl, and R² is 3- to 7-membered heterocycloalkyl optionally substituted with one or more hydroxy (e.g., morpholinyl or 1,3-dioxolan-2-yl). In some embodiments, one of R^{1a} and R^{1b} is 2-hydroxy-2-propyl, the other one of R^{1a} and R^{1b} is 1-hydroxy-1-propyl, and R² is 3- to 7-membered heterocycloalkyl optionally substituted with one or more hydroxy (e.g., morpholinyl or 1,3-dioxolan-2-yl). In some embodiments, one of R^{1a} and R^{1b} is 2-hydroxy-2-propyl, the other one of R^{1a} and R^{1b} is 2-hydroxy-1-propyl, and R² is 3- to 7-membered heterocycloalkyl optionally substituted with one or more hydroxy (e.g., morpholinyl or 1,3-dioxolan-2-yl). In some embodiments, one of R^{1a} and R^{1b} is 2-hydroxy-2-propyl, the other one of R^{1a} and R^{1b} is 3-hydroxy-1-propyl, and R² is 3- to 7-membered heterocycloalkyl optionally substituted with one or more hydroxy (e.g., morpholinyl or 1,3-dioxolan-2-yl). In some embodiments, one of R^{1a} and R^{1b} is 2-hydroxy-2-propyl, the other one of R^{1a} and R^{1b} is hydroxybutyl, and R² is 3- to 7-membered heterocycloalkyl optionally substituted with one or more hydroxy (e.g., morpholinyl or 1,3-dioxolan-2-yl). In some embodiments, one of R^{1a} and R^{1b} is 2-hydroxy-2-propyl, the other one of R^{1a} and R^{1b} is hydroxypentyl, and R² is 3- to 7-membered heterocycloalkyl optionally substituted with one or more hydroxy (e.g., morpholinyl or 1,3-dioxolan-2-yl). In some embodiments, one of R^{1a} and R^{1b} is 2-hydroxy-2-propyl, the other one of R^{1a} and R^{1b} is hydroxyhexyl, and R² is 3- to 7-membered heterocycloalkyl optionally substituted with one or more hydroxy (e.g., morpholinyl or 1,3-dioxolan-2-yl). In some embodiments, one of R^{1a} and R^{1b} is hydroxymethyl, the other one of R^{1a} and R^{1b} is hydroxymethyl, and R² is COCH₃. In some embodiments, one of R^{1a} and R^{1b} is hydroxymethyl, the other one of R^{1a} and R^{1b} is hydroxy ethyl, and R² is COCH₃. In some embodiments, one of R^{1a} and R^{1b} is hydroxymethyl, the other one of R^{1a} and R^{1b} is 2-hydroxy-2-propyl, and R² is COCH₃. In some embodiments, one of R^{1a} and R^{1b} is hydroxymethyl, the other one of R^{1a} and R^{1b} is 3-hydroxy-2-propyl, and R² is COCH₃. In some embodiments, one of R^{1a} and R^{1b} is hydroxymethyl, the other one of R^{1a} and R^{1b} is 1-hydroxy-1-propyl, and R² is COCH₃. In some embodiments, one of R^{1a} and R^{1b} is hydroxymethyl, the other one of R^{1a} and R^{1b} is 2-hydroxy-1-propyl, and R² is COCH₃. In some embodiments, one of R^{1a} and R^{1b} is hydroxymethyl, the other one of R^{1a} and R^{1b} is 3-hydroxy-1-propyl, and R² is COCH₃. In some embodiments, one of R^{1a} and R^{1b} is hydroxymethyl, the other one of R^{1a} and R^{1b} is hydroxybutyl, and R² is COCH₃. In some embodiments, one of R^{1a} and R^{1b} is hydroxymethyl, the other one of R^{1a} and R^{1b} is hydroxypentyl, and R² is COCH₃. In some embodiments, one of R^{1a} and R^{1b} is hydroxymethyl, the other one of R^{1a} and R^{1b} is hydroxyhexyl, and R² is COCH₃. In some embodiments, one of R^{1a} and R^{1b} is hydroxy ethyl, the other one of R^{1a} and R^{1b} is hydroxyethyl, and R² is COCH₃. In some embodiments, one of R^{1a} and R^{1b} is hydroxyethyl, the other one of R^{1a} and R^{1b} is 2-hydroxy-2-propyl, and R² is COCH₃. In some embodiments, one of R^{1a} and R^{1b} is hydroxyethyl, the other one of R^{1a} and R^{1b} is 3-hydroxy-2-propyl, and R² is COCH₃. In some embodiments, one of R^{1a} and R^{1b} is hydroxyethyl, the other one of R^{1a} and R^{1b} is 1-hydroxy-1-propyl, and R² is COCH₃. In some embodiments, one of R^{1a} and R^{1b} is hydroxyethyl, the other one of R^{1a} and R^{1b} is 2-hydroxy-1-propyl, and R² is COCH₃. In some embodiments, one of R^{1a} and R^{1b} is hydroxyethyl, the other one of R^{1a} and R^{1b} is 3-hydroxy-1-propyl, and R² is COCH₃. In some embodiments, one of R^{1a} and R^{1b} is hydroxyethyl, the other one of R^{1a} and R^{1b} is hydroxybutyl, and R² is COCH₃. In some embodiments, one of R^{1a} and R^{1b} is hydroxyethyl, the other one of R^{1a} and R^{1b} is hydroxypentyl, and R² is COCH₃. In some embodiments, one of R^{1a} and R^{1b} is hydroxyethyl, the other one of R^{1a} and R^{1b} is hydroxyhexyl, and R² is COCH₃. In some embodiments, one of R^{1a} and R^{1b} is 2-hydroxy-2-propyl, the other one of R^{1a} and R^{1b} is 2-hydroxy-2-propyl, and R² is COCH₃. In some embodiments, one of R^{1a} and R^{1b} is 2-hydroxy-2-propyl, the other one of R^{1a} and R^{1b} is 3-hydroxy-2-propyl, and R² is COCH₃. In some embodiments, one of R^{1a} and R^{1b} is 2-hydroxy-2-propyl, the other one of R^{1a} and R^{1b} is 1-hydroxy-1-propyl, and R² is COCH₃. In some embodiments, one of R^{1a} and R^{1b} is 2-hydroxy-2-propyl, the other one of R^{1a} and R^{1b} is 2-hydroxy-1-propyl, and R² is COCH₃. In some embodiments, one of R^{1a} and R^{1b} is 2-hydroxy-2-propyl, the other one of R^{1a} and R^{1b} is 3-hydroxy-1-propyl, and R² is COCH₃. In some embodiments, one of R^{1a} and R^{1b} is 2-hydroxy-2-propyl, the other one of R^{1a} and R^{1b} is hydroxybutyl, and R² is COCH₃. In some embodiments, one of R^{1a} and R^{1b} is 2-hydroxy-2-propyl, the other one of R^{1a} and R^{1b} is hydroxypentyl, and R² is COCH₃. In some embodiments, one of R^{1a} and R^{1b} is 2-hydroxy-2-propyl, the other one of R^{1a} and R^{1b} is hydroxyhexyl, and R² is COCH₃. In some embodiments, one of R^{1a} and R^{1b} is hydroxymethyl, the other one of R^{1a} and R^{1b} is hydroxymethyl, and R² is C₁-C₆ alkyl optionally substituted with one or more C₁-C₆ alkoxy. In some embodiments, one of R^{1a} and R^{1b} is hydroxymethyl, the other one of R^{1a} and R^{1b} is hydroxyethyl, and R² is C₁-C₆ alkyl optionally substituted with one or more C₁-C₆, alkoxy. In some embodiments, one of R^{1a} and R^{1b} is hydroxymethyl, the other one of R^{1a} and R^{1b} is 2-hydroxy-2-propyl, and R² is C₁-C₆ alkyl optionally substituted with one or more C₁-C₆, alkoxy. In some embodiments, one of R^{1a} and R^{1b} is hydroxymethyl, the other one of R^{1a} and R^{1b} is 3-hydroxy-2-propyl, and R² is C₁-C₆ alkyl optionally substituted with one or more C₁-C₆, alkoxy. In some embodiments, one of R^{1a} and R^{1b} is hydroxymethyl, the other one of R^{1a} and R^{1b} is 1-hydroxy-1-propyl, and R² is C₁-C₆ alkyl optionally substituted with one or more C₁-C₆, alkoxy. In some embodiments, one of R^{1a} and R^{1b} is hydroxymethyl, the other one of R^{1a} and R^{1b} is 2-hydroxy-1-propyl, and R² is C₁-C₆ alkyl optionally substituted with one or more C₁-C₆, alkoxy. In some embodiments, one of R^{1a} and R^{1b} is hydroxymethyl, the other one of R^{1a} and R^{1b} is 3-hydroxy-1-propyl, and R² is C₁-C₆ alkyl optionally substituted with one or more C₁-C₆, alkoxy. In some embodiments, one of R^{1a} and R^{1b} is hydroxymethyl, the other one of R^{1a} and R^{1b} is hydroxybutyl, and R² is C₁-C₆ alkyl optionally substituted with one or more C₁-C₆, alkoxy. In some embodiments, one of R^{1a} and R^{1b} is hydroxymethyl, the other one of R^{1a} and R^{1b} is hydroxypentyl, and R² is C₁-C₆ alkyl optionally substituted with one or more C₁-C₆ alkoxy. In some embodiments, one of R^{1a} and R^{1b} is hydroxymethyl, the other one of R^{1a} and R^{1b} is hydroxyhexyl, and R² is C₁-C₆ alkyl optionally substituted with one or more C₁-C₆ alkoxy. In some embodiments, one of R^{1a} and R^{1b} is hydroxy ethyl, the other one of R^{1a} and R^{1b} is hydroxyethyl, and R² is C₁-C₆ alkyl optionally substituted with one or more C₁-C₆ alkoxy. In some embodiments, one of R^{1a} and R^{1b} is hydroxyethyl, the other one of R^{1a} and R^{1b} is 2-hydroxy-2-propyl, and R² is C₁-C₆ alkyl optionally substituted with one or more C₁-C₆, alkoxy. In some embodiments, one of R^{1a} and R^{1b} is hydroxyethyl, the other one of R^{1a} and R^{1b} is 3-hydroxy-2-propyl, and R² is C₁-C₆ alkyl optionally substituted with one or more C₁-C₆, alkoxy. In some embodiments, one of R^{1a} and R^{1b} is hydroxyethyl, the other one of R^{1a} and R^{1b} is 1-hydroxy-1-propyl, and R² is C₁-C₆ alkyl optionally substituted with one or more C₁-C₆, alkoxy. In some embodiments, one of R^{1a} and R^{1b} is hydroxyethyl, the other one of R^{1a} and R^{1b} is 2-hydroxy-1-propyl, and R² is C₁-C₆ alkyl optionally substituted with one or more C₁-C₆, alkoxy. In some embodiments, one of R^{1a} and R^{1b} is hydroxyethyl, the other one of R^{1a} and R^{1b} is 3-hydroxy-1-propyl, and R² is C₁-C₆ alkyl optionally substituted with one or more C₁-C₆, alkoxy. In some embodiments, one of R^{1a} and R^{1b} is hydroxyethyl, the other one of R^{1a} and R^{1b} is hydroxybutyl, and R² is C₁-C₆ alkyl optionally substituted with one or more C₁-C₆, alkoxy. In some embodiments, one of R^{1a} and R^{1b} is hydroxyethyl, the other one of R^{1a} and R^{1b} is hydroxypentyl, and R² is C₁-C₆ alkyl optionally substituted with one or more C₁-C₆ alkoxy. In some embodiments, one of R^{1a} and R^{1b} is hydroxyethyl, the other one of R^{1a} and R^{1b} is hydroxyhexyl, and R² is C₁-C₆ alkyl optionally substituted with one or more C₁-C₆ alkoxy. In some embodiments, one of R^{1a} and R^{1b} is 2-hydroxy-2-propyl, the other one of R^{1a} and R^{1b} is 2-hydroxy-2-propyl, and R² C₁-C₆ alkyl optionally substituted with one or more C₁-C₆ alkoxy. In some embodiments, one of R^{1a} and R^{1b} is 2-hydroxy-2-propyl, the other one of R^{1a} and R^{1b} is 3-hydroxy-2-propyl, and R² C₁-C₆ alkyl optionally substituted with one or more C₁-C₆ alkoxy. In some embodiments, one of R^{1a} and R^{1b} is 2-hydroxy-2-propyl, the other one of R^{1a} and R^{1b} is 1-hydroxy-1-propyl, and R² C₁-C₆ alkyl optionally substituted with one or more C₁-C₆ alkoxy. In some embodiments, one of R^{1a} and R^{1b} is 2-hydroxy-2-propyl, the other one of R^{1a} and R^{1b} is 2-hydroxy-1-propyl, and R² is C₁-C₆ alkyl optionally substituted with one or more C₁-C₆, alkoxy. In some embodiments, one of R^{1a} and R^{1b} is 2-hydroxy-2-propyl, the other one of R^{1a} and R^{1b} is 3-hydroxy-1-propyl, and R² is C₁-C₆ alkyl optionally substituted with one or more C₁-C₆ alkoxy. In some embodiments, one of R^{1a} and R^{1b} is 2-hydroxy-2-propyl, the other one of R^{1a} and R^{1b} is hydroxybutyl, and R² is C₁-C₆ alkyl optionally substituted with one or more C₁-C₆, alkoxy. In some embodiments, one of R^{1a} and R^{1b} is 2-hydroxy-2-propyl, the other one of R^{1a} and R^{1b} is hydroxypentyl, and R² is C₁-C₆ alkyl optionally substituted with one or more C₁-C₆ alkoxy. In some embodiments, one of R^{1a} and R^{1b} is 2-hydroxy-2-propyl, the other one of R^{1a} and R^{1b} is hydroxyhexyl, and R² is C₁-C₆ alkyl optionally substituted with one or more C₁-C₆ alkoxy. In some embodiments, R^{1a} is different from R^{1b}. In some embodiments, R^{1a} is the same as R^{1b}. In some embodiments, R^{1a} and R² are different. In some embodiments, R^{1b} and R² are different. In some embodiments, R^{1a} is the same as R^{1b}, and R^{1a} is different from R². In some embodiments, R^{1a} is different from R^{1b}, and one of R^{1a} and R^{1b} is the same as R². In some embodiments, R^{1a} is different from R^{1b}, and both R^{1a} and R^{1b} are different from R². In some embodiments, R² comprises a carbonyl group. In some embodiments, R² comprises 1 or 2 (e.g., 1) nitrogen atoms. In some embodiments, R² comprises 1 or 2 (e.g., 1) oxygen atoms. In some embodiments, R² comprises a sulfur atom. In some embodiments, R² comprises a carbonyl group. In some embodiments, R² comprises a sulfur atom. In some embodiments, R^{1a} is ortho to R^{1b}. In some embodiments, R^{1a} is meta to R^{1b}. In some embodiments, R^{1a} is para to R^{1b}.

### The Variables o and p

In some embodiments, o=1 or 2. In some embodiments, o=1. In some embodiments, o=2. In some embodiments, p=0, 1, 2, or 3. In some embodiments, p=0. In some embodiments, p=1. In some embodiments, p=2. In some embodiments, o=1 and p=0. In some embodiments, o=2 and p=0. In some embodiments, o=1 and p=1. In some embodiments, o=1 and p=2. In some embodiments, o=2 and p=1. In some embodiments, o=2 and p=2. In some embodiments, o=2 and p=3.

### The Ring B and Substitutions on the Ring B

In some embodiments, B is a 5- to 10-membered monocyclic or bicyclic heteroaryl or a C₆-C₁₀ monocyclic or bicyclic aryl, such as phenyl. In some embodiments, B is a 5- to 6-membered monocyclic heteroaryl or a C₆ monocyclic aryl. In some embodiments, B is a 5- to 10-membered monocyclic or bicyclic heteroaryl. In some embodiments, B is a C₆-C₁₀ monocyclic or bicyclic aryl. In some embodiments, B is phenyl substituted with 1 or 2 R⁶ and optionally substituted with 1, 2, or 3 R⁷. In some embodiments, B is pyridyl substituted with 1 or 2 R⁶ and optionally substituted with 1, 2, or 3 R⁷. In some embodiments, B is phenyl, o is 1 or 2, and p is 0, 1, 2 or 3. In some embodiments, B is pyridyl, o is 1 or 2, and p is 0, 1, 2 or 3. In some embodiments, B is phenyl, o is 1 or 2, and p is 0. In some embodiments, B is pyridyl, o is 1 or 2, and p is 0. In some embodiments, B is phenyl, o is 1 or 2, and p is 1. In some embodiments, B is pyridyl, o is 1 or 2, and p is 1. In some embodiments, B is phenyl, o is 1, and p is 0, 1, 2 or 3. In some embodiments, B is phenyl, o is 2, and p is 0, 1, 2 or 3. In some embodiments, B is pyridyl, o is 1, and p is 0, 1, 2 or 3. In some embodiments, B is pyridyl, o is 2, and p is 0, 1, 2 or 3. In some embodiments, B is phenyl, o is 1, and p is 0 or 1. In some embodiments, B is phenyl, o is 2, and p is 0 or 1. In some embodiments, B is pyridyl, o is 1, and p is 0 or 1. In some embodiments, B is pyridyl, o is 2, and p is 0 or 1. In some embodiments, B is one of the rings disclosed hereinbelow, substituted as disclosed hereinbelow, wherein in each case the bond that is shown as being broken by the wavy line [symbol] connects B to the CR⁴R⁵ group of Formula AA. In some embodiments, the optionally substituted ring B

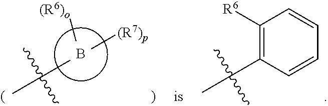

In some embodiments, the optionally substituted ring B

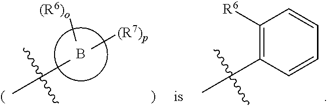

In some embodiments, the optionally substituted ring B is

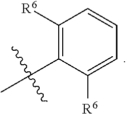

In some embodiments, the optionally substituted ring B is

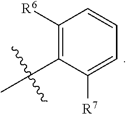

In some embodiments, the optionally substituted ring B is

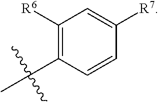

In some embodiments, the optionally substituted ring B is

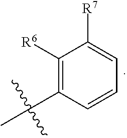

In some embodiments, the optionally substituted ring B is

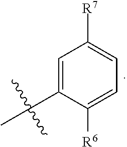

In some embodiments, the optionally substituted ring B is

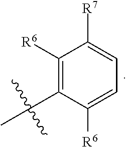

In some embodiments, the optionally substituted ring B is

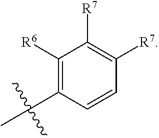

In some embodiments, the optionally substituted ring B is

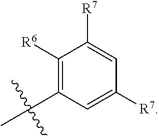

In some embodiments, the optionally substituted ring B is

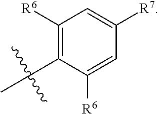

In some embodiments, the optionally substituted ring B is

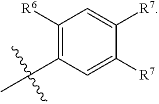

In some embodiments, the optionally substituted ring B is

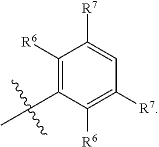

In some embodiments, the optionally substituted ring B is

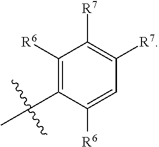

In some embodiments, the optionally substituted ring B is

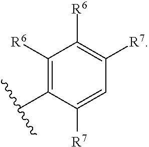

In some embodiments, the optionally substituted ring B is

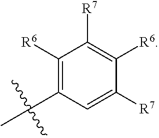

In some embodiments, the optionally substituted ring B is

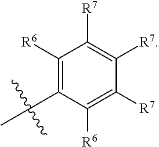

In some embodiments, the optionally substituted ring B is

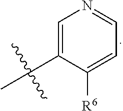

In some embodiments, the optionally substituted ring B is

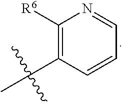

In embodiments, the optionally substituted ring B is

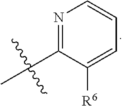

In some embodiments, the optionally substituted ring B is

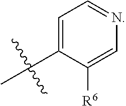

In some embodiments, the optionally substituted ring B is

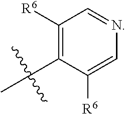

The groups R⁶, R^{6′}, R⁷, and R⁷′

In some embodiments,

R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₃-C₆ alkyl, C₃-C₁₀ cycloalkyl and 3- to 10-membered heterocycloalkyl, and a C₂-C₆ alkenyl,
wherein R⁶ and R⁷ are each optionally substituted with one or more substituents independently selected from
hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl,
C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, halo, C₆-C₁₀ aryl or NR⁸R⁹, or wherein R⁶ or R⁷ is optionally fused to a five-to-seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments,

R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₇ cycloalkyl and 3- to 7-membered heterocycloalkyl,
wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments,

R⁶ and R⁷ are each independently selected from C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₇ cycloalkyl and 3- to 7-membered heterocycloalkyl,
wherein the C₃-C₇ cycloalkyl, C₁-C₆ haloalkyl, and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments,

R⁶ and R⁷ are each independently selected from C₁-C₆ alkyl, halo, CN, NO₂, COC₁-C₆ alkyl, CO₂C₁-C₆ alkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₇ cycloalkyl and 3- to 7-membered heterocycloalkyl,
wherein the C₁-C₆ alkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments,

R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₇ cycloalkyl and 3- to 7-membered heterocycloalkyl,
wherein the C₁-C₆ alkyl, C₁-C₆ haloalkyl, C₃-C₇ cycloalkyl, and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are unsubstituted;
   or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments,

R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₇ cycloalkyl and 3- to 7-membered heterocycloalkyl,
wherein the C₁-C₆ alkyl, C₃-C₇ cycloalkyl and 3- to 7-membered heterocycloalkyl are each unsubstituted;
or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments,

R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
and R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
   or R⁶ and R⁷, taken together with the atoms connecting them, independently form C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments,

R⁶ and R⁷ are each independently selected from C₁-C₆ alkyl, C₁-C₆ alkoxy, halo, CN, NO₂, COC₁-C₆ alkyl, CO₂C₁-C₆ alkyl, C₆-C₁₀ aryl, 5- to 10-membered heteroaryl, CONR⁸R⁹, and 3- to 7-membered heterocycloalkyl,
wherein the C₁-C₆ alkyl and 3- to 7-membered heterocycloalkyl is optionally substituted with one or more substituents each independently selected from hydroxy or oxo, or at least one pair of R⁶ and R⁷ on adjacent atoms, taken together with the atoms connecting them, independently form at least one C₄-C₈ carbocyclic ring, wherein the carbocyclic ring is optionally independently substituted with one or more hydroxy or oxo.

In some embodiments, at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments, R⁶ and R⁷ are each independently selected from CN, C₁-C₆ alkyl, 5- to 10-membered heteroaryl, and 3- to 7-membered heterocycloalkyl;
wherein the C₁-C₆ alkyl is optionally substituted with one or more substituents each independently selected from hydroxyl or C₁-C₆, alkoxy.

In some embodiments, R⁶ is C₁-C₆ alkyl. In some embodiments, R⁶ is isopropyl. In some embodiments, R⁶ is n-propyl. In some embodiments, R⁶ is butyl (e.g., s-butyl, iso-butyl). In some embodiments, R⁶ is C₃-C₁₀ cycloalkyl. In some embodiments, R⁶ is cyclopropyl. In some embodiments, R⁶ is halo. In some embodiments, R⁶ is CN. In some embodiments, R⁶ is C₁-C₆ alkyl substituted with hydroxyl (e.g., hydroxymethyl, hydroxyethyl, or 2-hydroxy-2-propyl. In some embodiments, R⁶ is C₁-C₆ alkyl substituted with C₁-C₆, alkoxy (e.g., methoxymethyl). In some embodiments, R⁶ is C₁-C₆ alkyl substituted with C₁-C₆, alkoxy (e.g., methoxymethyl) In some embodiments, R⁶ is C₁-C₆ alkyl substituted with O(C₃-C₁₀ cycloalkyl)

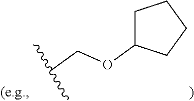 [SMILES: *COC1CCCC1]

In some embodiments, R⁶ is C₆-C₁₀ aryl, optionally fused to a five-to-seven-membered carbocyclic ring or heterocyclic ring containing one or two heteroatoms independently selected from oxygen, sulfur and nitrogen. In some embodiments, R⁶ is phenyl, optionally fused to a five-to-seven-membered carbocyclic ring or heterocyclic ring containing one or two heteroatoms independently selected from oxygen, sulfur and nitrogen. For example, R⁶ is

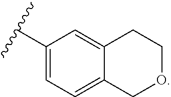 [SMILES: *c1ccc2c(c1)CCOC2]

In some embodiments, R⁶ is imidazolyl. In some embodiments, R⁶ is pyrazolyl. In some embodiments, R⁶ is pyrrolyl. In some embodiments, R⁶ is thiazolyl. In some embodiments, R⁶ is isothiazolyl. In some embodiments, R⁶ is oxazolyl. In some embodiments, R⁶ is isoxazolyl. In some embodiments, R⁶ is pyridyl. In some embodiments, R⁶ is pyrimidinyl. In some embodiments, R⁷ is C₁-C₆ alkyl. In some embodiments, R⁷ is isopropyl. In some embodiments, R⁷ is n-propyl. In some embodiments, R⁷ is butyl (e.g., s-butyl, iso-butyl). In some embodiments, R⁷ is C₃-C₁₀ cycloalkyl. In some embodiments, R⁷ is cyclopropyl. In some embodiments, R⁷ is halo. In some embodiments, R⁷ is CN. In some embodiments, R⁷ is C₁-C₆ alkyl substituted with hydroxyl (e.g., hydroxymethyl, hydroxyethyl, or 2-hydroxy-2-propyl. In some embodiments, R⁷ is C₁-C₆ alkyl substituted with C₁-C₆, alkoxy (e.g., methoxymethyl) In some embodiments, R⁷ is C₁-C₆ alkyl substituted with O(C₃-C₁₀ cycloalkyl)

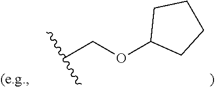 [SMILES: *COC1CCCC1]

In some embodiments, R⁷ is C₆-C₁₀ aryl, optionally fused to a five-to-seven-membered carbocyclic ring or heterocyclic ring containing one or two heteroatoms independently selected from oxygen, sulfur and nitrogen. In some embodiments, R⁷ is phenyl, optionally fused to a five-to-seven-membered carbocyclic ring or heterocyclic ring containing one or two heteroatoms independently selected from oxygen, sulfur and nitrogen. For example, R⁷ is

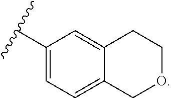 [SMILES: *c1ccc2c(c1)CCOC2]

In some embodiments, R⁷ is imidazolyl. In some embodiments, R⁷ is pyrazolyl. In some embodiments, R⁷ is pyrrolyl. In some embodiments, R⁷ is thiazolyl. In some embodiments, R⁷ is isothiazolyl. In some embodiments, R⁷ is oxazolyl. In some embodiments, R⁷ is isoxazolyl. In some embodiments, R⁷ is pyridyl. In some embodiments, R⁷ is pyrimidinyl. In some embodiments, o=1; p=0; and
R⁶ is selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₇ cycloalkyl and 3- to 7-membered heterocycloalkyl,
wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl.

In some embodiments, o=1; p=0; and

R⁶ is selected from C₁-C₆ alkyl, C₁-C₆ alkoxy, halo, CN, NO₂, COC₁-C₆ alkyl, CO₂C₁-C₆ alkyl, C₆-C₁₀ aryl, 5- to 10-membered heteroaryl, CONR⁸R⁹, and 3- to 7-membered heterocycloalkyl, wherein the C₁-C₆ alkyl and 3- to 7-membered heterocycloalkyl is optionally substituted with one or more substituents each independently selected from hydroxy or oxo.

In some embodiments, o=1 or 2; p=1, 2, or 3; and

R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₇ cycloalkyl and 3- to 7-membered heterocycloalkyl,
wherein the C₁-C₆ alkyl, C₁-C₆ haloalkyl, C₃-C₇ cycloalkyl, and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl.

In some embodiments, o=2; p=1; and

each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
and R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
   or R⁶ and R⁷, taken together with the atoms connecting them, independently form C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2 or 3; and

each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
   or at least one pair of R⁶ and R⁷ on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments, o=1 or 2; p=1, 2, or 3; and

R⁶ and R⁷ are each independently selected from C₁-C₆ alkyl, C₁-C₆ alkoxy, halo, CN, NO₂, COC₁-C₆ alkyl, CO₂C₁-C₆ alkyl, C₆-C₁₀ aryl, 5- to 10-membered heteroaryl, CONR⁸R⁹, and 3- to 7-membered heterocycloalkyl,
wherein the C₁-C₆ alkyl and 3- to 7-membered heterocycloalkyl is optionally substituted with one or more substituents each independently selected from hydroxy or oxo, or at least one pair of R⁶ and R⁷ on adjacent atoms, taken together with the atoms connecting them, independently form at least one C₄-C₈ carbocyclic ring, wherein the carbocyclic ring is optionally independently substituted with one or more hydroxy or oxo.

In some embodiments, o=1 or 2; p=1, 2, or 3; and

R⁶ and R⁷ are each independently selected from C₁-C₆ alkyl, C₁-C₆ alkoxy, halo, CN, NO₂, COC₁-C₆ alkyl, CO₂C₁-C₆ alkyl, C₆-C₁₀ aryl, 5- to 10-membered heteroaryl, CONR⁸R⁹, and 3- to 7-membered heterocycloalkyl,
wherein the C₁-C₆ alkyl and 3- to 7-membered heterocycloalkyl is optionally substituted with one or more substituents each independently selected from hydroxy or oxo.

In some embodiments, o=1 or 2; p=1, 2, or 3; and

one R⁶ and one R⁷ are on adjacent atoms, and taken together with the atoms connecting them, form a C₄-C₈ carbocyclic ring or a 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=1 or 2; p=1, 2, or 3; and

one R⁶ and one R⁷ are on adjacent atoms, and taken together with the atoms connecting them, form a C₆ carbocyclic ring or a 5-to-6-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=1 or 2; p=1, 2, or 3; and

one R⁶ and one R⁷ are on adjacent atoms, and taken together with the atoms connecting them, form a C₄-C₈ carbocyclic ring or a 5- to 8-membered heterocyclic ring containing 1 or 2 heteroatoms independently selected from O, N, and S, wherein the carbocyclic ring or heterocyclic ring is unsubstituted.

In some embodiments, o=2; p=2 or 3; and

two pairs, each of one R⁶ and one R⁷, are on adjacent atoms, and each pair of one R⁶ and one R⁷ taken together with the atoms connecting them independently form a C₄-C₈ carbocyclic ring or a 5- to 8-membered heterocyclic ring containing 1 or 2 heteroatoms independently selected from O, N, and S, wherein each carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2 or 3; and

two pairs, each of one R⁶ and one R⁷, are on adjacent atoms, and each pair of one R⁶ and one R⁷ taken together with the atoms connecting them independently form a C₆ carbocyclic ring or a 5-to-6-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2 or 3; and

two pairs, each of one R⁶ and one R⁷, are on adjacent atoms, and each pair of one R⁶ and one R⁷ taken together with the atoms connecting them independently form a C₄-C₈ carbocyclic ring or a 5- to 8-membered heterocyclic ring containing 1 or 2 heteroatoms independently selected from O, N, and S, wherein the carbocyclic ring or heterocyclic ring is unsubstituted.

### Particular Embodiments Wherein o=1; p=0

In some embodiments, R⁶ is C₁-C₆ alkyl. In some embodiments, R⁶ is isopropyl. In some embodiments, R⁶ is ethyl. In some embodiments, R⁶ is methyl. In some embodiments, R⁶ is isopropyl. In some embodiments, R⁶ is n-propyl. In some embodiments, R⁶ is butyl (e.g., s-butyl, iso-butyl). In some embodiments, R⁶ is C₁-C₆ alkyl substituted with one or more halo. In some embodiments, R⁶ is trifluoromethyl. In some embodiments, R⁶ is trifluoromethoxy. In some embodiments, R⁶ is C₃-C₇ cycloalkyl. In some embodiments, R⁶ is cyclopropyl. In some embodiments, R⁶ is halo. In some embodiments, R⁶ is chloro. In some embodiments, R⁶ is fluoro. In some embodiments, R⁶ is cyano. In some embodiments, R⁶ is attached to a carbon of an aryl ring B. In some embodiments, R⁶ is attached to a carbon of a heteroaryl ring B. In some embodiments, R⁶ is attached to a nitrogen of a heteroaryl ring B.

### Particular Embodiments Wherein o=1 or 2; p=1, 2, or 3

In some embodiments, at least one R⁶ is C₁-C₆ alkyl, and at least one R⁷ is C₁-C₆ alkyl optionally substituted with one or more halo. In some embodiments, at least one R⁶ is C₁-C₆ alkyl and at least one R⁷ is C₁-C₆ alkyl. In some embodiments, at least one R⁶ is isopropyl and at least one R⁷ is methyl. In some embodiments, at least one R⁶ is isopropyl and at least one R⁷ is isopropyl. In some embodiments, at least one R⁶ is isopropyl and at least one R⁷ is n-propyl. In some embodiments, at least one R⁶ is isopropyl and at least one R⁷ is sec-butyl. In some embodiments, at least one R⁶ is isopropyl and at least one R⁷ is iso-butyl. In some embodiments, o=1; p=1; R⁶ is isopropyl; and R⁷ is isopropyl. In some embodiments, o=2; p=1, 2, or 3; one R⁶ is isopropyl; and one R⁷ is isopropyl. In some embodiments, o=2; p=1, 2, or 3; one R⁶ is isopropyl; and one R⁷ is n-propyl. In some embodiments, o=2; p=1, 2, or 3; one R⁶ is isopropyl; and one R⁷ is iso-butyl. In some embodiments, o=2; p=1, 2, or 3; one R⁶ is isopropyl; and one R⁷ is sec-butyl. In certain of the foregoing embodiments (when o=2; p=1, 2, or 3; one R⁶ is isopropyl; and one R⁷ is isopropyl, n-propyl, iso-butyl, or sec-butyl), the other R⁶ is cyano. In certain of the foregoing embodiments (when o=2; p=1, 2, or 3; one R⁶ is isopropyl; and one R⁷ is isopropyl, n-propyl, iso-butyl, or sec-butyl), the other R⁶ is halo. In some embodiments, at least one R⁶ is C₁-C₆ alkyl, and at least one R⁷ is C₁-C₆ alkyl substituted with one or more halo. In some embodiments, at least one R⁶ is isopropyl and at least one R⁷ is trifluoromethyl. In some embodiments, at least one R⁶ is C₁-C₆ alkyl, and at least one R⁷ is C₃-C₇ cycloalkyl. In some embodiments, at least one R⁶ is isopropyl and at least one R⁷ is cyclopropyl. In some embodiments, o=1; p=1; R⁶ is isopropyl; and R⁷ is cyclopropyl. In some embodiments, o=2; p=1, 2, or 3; one R⁶ is isopropyl; and one R⁷ is cyclopropyl. In certain of the foregoing embodiments (when o=2; p=1, 2, or 3; one R⁶ is isopropyl; and one R⁷ is cyclopropyl), the other R⁶ is halo. In some embodiments, at least one R⁶ is C₁-C₆ alkyl, and at least one R⁷ is halo. In some embodiments, at least one R⁶ is isopropyl and at least one R⁷ is halo. In some embodiments, at least one R⁶ is isopropyl and at least one R⁷ is chloro. In some embodiments, at least one R⁶ is isopropyl and at least one R⁷ is fluoro. In some embodiments, o=1; p=1; R⁶ is isopropyl; and R⁷ is chloro. In some embodiments, o=2; p=1; at least one R⁶ is isopropyl; and R⁷ is chloro. In some embodiments, o=1; p=1; R⁶ is isopropyl; and R⁷ is fluoro. In some embodiments, o=2; p=1; at least one R⁶ is isopropyl; and R⁷ is fluoro. In some embodiments, o=2; p=2; at least one R⁶ is isopropyl; and at least one R⁷ is fluoro. In some embodiments, o=2; p=2; at least one R⁶ is isopropyl; one R⁷ is fluoro; and the other R⁷ is cyano. In certain of the foregoing embodiments, the other R⁶ is isopropyl; the other R⁶ is n-propyl; the other R⁶ is iso-butyl; the other R⁶ is cyclopropyl; or the other R⁶ is sec-butyl. In some embodiments, o=2; p=3; at least one R⁶ is isopropyl; two R⁷ are fluoro; and one R⁷ is chloro. In some embodiments, o=2; p=3; at least one R⁶ is isopropyl; two R⁷ are fluoro; and one R⁷ is cyano. In some embodiments, o=2; p=1; at least one R⁶ is ethyl; and R⁷ is fluoro. In some embodiments, o=2; p=1; one R⁶ is isopropyl; the other R⁶ is trifluoromethyl; and R⁷ is chloro. In some embodiments, at least one R⁶ is C₁-C₆ alkyl, and at least one R⁷ is cyano. In some embodiments, at least one R⁶ is isopropyl and at least one R⁷ is cyano. In some embodiments, o=1; p=1; R⁶ is isopropyl; and R⁷ is cyano. In some embodiments, o=2; p=1; at least one R⁶ is isopropyl; and R⁷ is cyano. In some embodiments, at least one R⁶ is C₃-C₇ cycloalkyl, and at least one R⁷ is C₃-C₇ cycloalkyl. In some embodiments, at least one R⁶ is cyclopropyl, and at least one R⁷ is cyclopropyl. In some embodiments, at least one R⁶ is C₃-C₇ cycloalkyl, and at least one R⁷ is halo. In some embodiments, at least one R⁶ is cyclopropyl and at least one R⁷ is halo. In some embodiments, at least one R⁶ is cyclopropyl and at least one R⁷ is chloro. In some embodiments, at least one R⁶ is cyclopropyl and at least one R⁷ is fluoro. In some embodiments, o=1; p=1; R⁶ is cyclopropyl; and R⁷ is chloro. In some embodiments, o=1; p=1; R⁶ is cyclopropyl; and R⁷ is fluoro. In some embodiments, at least one R⁶ is C₁-C₆ alkyl, and at least one R⁷ is C₁-C₆ alkoxy optionally substituted with one or more halo. In some embodiments, at least one R⁶ is isopropyl, and at least one R⁷ is C₁-C₆ alkoxy. In some embodiments, at least one R⁶ is isopropyl, and at least one R⁷ is methoxy. In some embodiments, o=1; p=1; R⁶ is isopropyl, and R⁷ is methoxy. In some embodiments, o=2; p=1; at least one R⁶ is isopropyl, and R⁷ is methoxy. In some embodiments, at least one R⁶ is C₁-C₆ alkyl, and at least one R⁷ is C₁-C₆ alkoxy substituted with one or more halo. In some embodiments, at least one R⁶ is isopropyl, and at least one R⁷ is trifluoromethoxy. In some embodiments, at least one R⁶ is isopropyl, and at least one R⁷ is difluoromethoxy. In some embodiments, at least one R⁶ is halo, and at least one R⁷ is C₁-C₆ haloalkyl optionally substituted with hydroxy. In some embodiments, at least one R⁶ is C₁-C₆ alkyl, and at least one R⁷ is C₁-C₆ alkyl which is optionally substituted with O(C₃-C₁₀ cycloalkyl). In some embodiments, at least one R⁶ is isopropyl, and at least one R⁷ is C₁-C₆ alkyl which is optionally substituted with O(C₃-C₁₀ cycloalkyl). In some embodiments, at least one R⁶ is isopropyl, and at least one R⁷ is

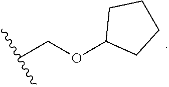 [SMILES: *COC1CCCC1]

In some embodiments, at least one R⁶ is C₁-C₆ alkyl, and at least one R⁷ is C₁-C₆ alkyl which is optionally substituted with C₁-C₆ alkoxy. In some embodiments, at least one R⁶ is isopropyl, and at least one R⁷ is C₁-C₆ alkyl which is optionally substituted with C₁-C₆ alkoxy. In some embodiments, at least one R⁶ is isopropyl, and at least one R⁷ is C₁-C₆ alkyl which is optionally substituted with methoxy. In some embodiments, at least one R⁶ is isopropyl, and at least one R⁷ is C₁-C₆ alkyl which is optionally substituted with methoxymethyl.

In some embodiments, o=1; p=1; R⁶ is chloro, and R⁷ is trifluoromethyl. In some embodiments, at least one R⁶ is halo, and at least one R⁷ is C₁-C₆ haloalkoxy. In some embodiments, at least one R⁶ is chloro, and at least one R⁷ is trifluoromethoxy. In some embodiments, o=1; p=1; R⁶ is chloro, and R⁷ is trifluoromethoxy. In some embodiments, at least one R⁶ is C₁-C₆, alkoxy; and at least one R⁷ is halo. In some embodiments, o=1; p=2; R⁶ is C₁-C₆ alkoxy; and at least one R⁷ is chloro. In some embodiments, at least one R⁶ is C₁-C₆ alkyl, and at least one R⁷ is C₆-C₁₀ aryl, optionally optionally fused to a five-to-seven-membered carbocyclic ring or heterocyclic ring containing one or two heteroatoms independently selected from oxygen, sulfur and nitrogen. In some embodiments, at least one R⁶ is isopropyl, and at least one R⁷ is C₆-C₁₀ aryl, optionally optionally fused to a five-to-seven-membered carbocyclic ring or heterocyclic ring containing one or two heteroatoms independently selected from oxygen, sulfur and nitrogen. In some embodiments, at least one R is isopropyl, and at least one R⁷ is

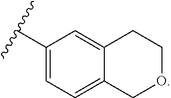 [SMILES: *c1ccc2c(c1)CCOC2]

In some embodiments, at least one R⁷ is C₁-C₆ alkyl, and at least one R⁶ is C₁-C₆ alkyl optionally substituted with one or more halo. In some embodiments, at least one R⁷ is isopropyl and at least one R⁶ is methyl. In some embodiments, at least one R⁷ is isopropyl and at least one R⁶ is isopropyl. In some embodiments, at least one R⁷ is isopropyl and at least one R⁶ is n-propyl. In some embodiments, at least one R⁷ is isopropyl and at least one R⁶ is s-butyl. In some embodiments, at least one R⁷ is isopropyl and at least one R⁶ is iso-butyl. In some embodiments, o=1; p=1; R⁷ is isopropyl; and R⁶ is isopropyl. In some embodiments, o=2; p=1, 2, or 3; one R⁷ is isopropyl; and one R⁶ is isopropyl. In some embodiments, o=2; p=1, 2, or 3; one R⁷ is isopropyl; and one R⁶ is n-propyl. In some embodiments, o=2; p=1, 2, or 3; one R⁷ is isopropyl; and one R⁶ is iso-butyl. In some embodiments, o=2; p=1, 2, or 3; one R⁷ is isopropyl; and one R⁶ is sec-butyl. In certain of the foregoing embodiments (when o=2; p=1, 2, or 3; one R⁷ is isopropyl; and one R⁶ is isopropyl, n-propyl, iso-butyl, or sec-butyl), the other R⁶ is cyano. In certain of the foregoing embodiments (when o=2; p=1, 2, or 3; one R⁷ is isopropyl; and one R⁶ is isopropyl, n-propyl, iso-butyl, or sec-butyl), the other R⁶ is halo. In some embodiments, at least one R⁷ is C₁-C₆ alkyl, and at least one R⁶ is C₁-C₆ alkyl substituted with one or more halo. In some embodiments, at least one R⁷ is isopropyl and at least one R⁶ is trifluoromethyl. In some embodiments, at least one R⁷ is C₁-C₆ alkyl, and at least one R⁶ is C₃-C₇ cycloalkyl. In some embodiments, at least one R⁷ is isopropyl and at least one R⁶ is cyclopropyl. In some embodiments, o=1; p=1; R⁷ is isopropyl; and R⁶ is cyclopropyl. In some embodiments, o=2; p=1, 2, or 3; one R⁷ is isopropyl; and one R⁶ is cyclopropyl. In some embodiments, o=2; p=1, 2, or 3; one R⁷ is isopropyl; and one R⁶ is cyclopropyl. In certain of the foregoing embodiments (when o=2; p=1, 2, or 3; one R⁷ is isopropyl; and one R⁶ is cyclopropyl), the other R⁶ is halo. In some embodiments, at least one R⁷ is C₁-C₆ alkyl, and at least one R⁶ is halo. In some embodiments, at least one R⁷ is isopropyl and at least one R⁶ is halo. In some embodiments, at least one R⁷ is isopropyl and at least one R⁶ is chloro. In some embodiments, at least one R⁷ is isopropyl and at least one R⁶ is fluoro. In some embodiments, o=1; p=1; R⁷ is isopropyl; and R⁶ is chloro. In some embodiments, o=2; p=1; R⁷ is isopropyl; and at least one R⁶ is chloro. In some embodiments, o=1; p=1; R⁷ is isopropyl; and R⁶ is fluoro. In some embodiments, o=2; p=1; R⁷ is isopropyl; and at least one R⁶ is fluoro. In some embodiments, o=2; p=2; at least one R⁷ is isopropyl; and at least one R⁶ is fluoro. In some embodiments, o=2; p=2; at least one R⁷ is isopropyl; one R⁶ is fluoro; and the other R⁶ is cyano. In some embodiments, o=2; p=1; R⁷ is ethyl; and at least one R⁶ is fluoro. In some embodiments, o=1; p=2; one R⁷ is isopropyl; the other R⁷ is trifluoromethyl; and R⁶ is chloro. In some embodiments, at least one R⁷ is C₁-C₆ alkyl, and at least one R⁶ is cyano. In some embodiments, at least one R⁷ is isopropyl and at least one R⁶ is cyano. In some embodiments, o=1; p=1; R⁷ is isopropyl; and R⁶ is cyano. In some embodiments, o=2; p=1; R⁷ is isopropyl; and at least one R⁶ is cyano. In some embodiments, at least one R⁷ is C₃-C₇ cycloalkyl, and at least one R⁶ is C₃-C₇ cycloalkyl. In some embodiments, at least one R⁷ is cyclopropyl, and at least one R⁶ is cyclopropyl. In some embodiments, at least one R⁷ is C₃-C₇ cycloalkyl, and at least one R⁶ is halo. In some embodiments, at least one R⁷ is cyclopropyl and at least one R⁶ is halo. In some embodiments, at least one R⁷ is cyclopropyl and at least one R⁶ is chloro. In some embodiments, at least one R⁷ is cyclopropyl and at least one R⁶ is fluoro. In some embodiments, o=1; p=1; R⁷ is cyclopropyl; and R⁶ is chloro. In some embodiments, o=1; p=1; R⁷ is cyclopropyl; and R⁶ is fluoro. In some embodiments, at least one R⁷ is C₁-C₆ alkyl, and at least one R⁶ is C₁-C₆ alkoxy optionally substituted with one or more halo. In some embodiments, at least one R⁷ is isopropyl, and at least one R⁶ is C₁-C₆ alkoxy. In some embodiments, at least one R⁷ is isopropyl, and at least one R⁶ is methoxy. In some embodiments, o=1; p=1; R⁷ is isopropyl, and R⁶ is methoxy. In some embodiments, o=2; p=1; R⁷ is isopropyl, and at least one R⁶ is methoxy. In some embodiments, at least one R⁷ is C₁-C₆ alkyl, and at least one R⁶ is C₁-C₆ alkoxy substituted with one or more halo. In some embodiments, at least one R⁷ is isopropyl, and at least one R⁶ is trifluoromethoxy. In some embodiments, at least one R⁷ is halo, and at least one R⁶ is C₁-C₆ haloalkyl optionally substituted with one or more hydroxy. In some embodiments, at least one R⁶ is C₁-C₆ alkyl, and at least one R⁷ is C₁-C₆ alkyl which is optionally substituted with O(C₃-C₁₀ cycloalkyl). In some embodiments, at least one R⁶ is isopropyl, and at least one R⁷ is C₁-C₆ alkyl which is optionally substituted with O(C₃-C₁₀ cycloalkyl). In some embodiments, at least one R⁶ is isopropyl, and at least one R⁷ is

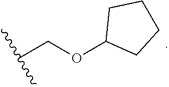 [SMILES: *COC1CCCC1]

In some embodiments, o=1; p=1; R⁷ is chloro, and R⁶ is trifluoromethyl. In some embodiments, at least one R⁷ is halo, and at least one R⁶ is C₁-C₆ haloalkoxy. In some embodiments, at least one R⁷ is chloro, and at least one R⁶ is trifluoromethoxy. In some embodiments, o=1; p=1; R⁷ is chloro, and R⁶ is trifluoromethoxy. In some embodiments, at least one R⁷ is C₁-C₆, alkoxy; and at least one R⁶ is halo. In some embodiments, o=1; p=2; at least one R⁷ is C₁-C₆ alkoxy; and R⁶ is chloro. In some embodiments, at least one R⁷ is C₁-C₆ alkyl, and at least one R⁶ is C₆-C₁₀ aryl, optionally optionally fused to a five-to-seven-membered carbocyclic ring or heterocyclic ring containing one or two heteroatoms independently selected from oxygen, sulfur and nitrogen. In some embodiments, at least one R⁷ is isopropyl, and at least one R⁶ is C₆-C₁₀ aryl, optionally optionally fused to a five-to-seven-membered carbocyclic ring or heterocyclic ring containing one or two heteroatoms independently selected from oxygen, sulfur and nitrogen. In some embodiments, at least one R⁷ is isopropyl, and at least one R⁶ is

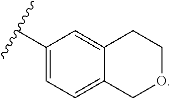 [SMILES: *c1ccc2c(c1)CCOC2]

In some embodiments, R⁶ and R⁷ are each attached to a carbon of an aryl ring B. In some embodiments, R⁶ and R⁷ are each attached to a carbon of a heteroaryl ring B. In some embodiments, R⁶ is attached to a carbon and R⁷ is attached to a nitrogen of a heteroaryl ring B. In some embodiments, R⁷ is attached to a carbon and R⁶ is attached to a nitrogen of a heteroaryl ring B. In some embodiments, one R⁶ and one R⁷ are on adjacent atoms, and taken together with the atoms connecting them, form a C₅ carbocyclic ring optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹. In some embodiments, R⁶ and R⁷ are on adjacent atoms, and taken together with the atoms connecting them, form a C₅ aliphatic carbocyclic ring. In some embodiments, R⁶ and R⁷ are on adjacent atoms, and taken together with the atoms connecting them, form a C₆ carbocyclic ring optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹. In some embodiments, R⁶ and R⁷ are on adjacent atoms, and taken together with the atoms connecting them, form a C₆ aliphatic carbocyclic ring. In some embodiments, R⁶ and R⁷ are on adjacent atoms, and taken together with the atoms connecting them, form a C₆ aromatic carbocyclic ring. In some embodiments, R⁶ and R⁷ are on adjacent atoms, and taken together with the atoms connecting them, form a 5-membered heterocyclic ring containing 1 or 2 heteroatoms independently selected from O, N, and S, optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹. In some embodiments, R⁶ and R⁷ are on adjacent atoms, and taken together with the atoms connecting them, form a 5-membered aliphatic heterocyclic ring containing 1 or 2 heteroatoms independently selected from O, N, and S. In some embodiments, R⁶ and R⁷ are on adjacent atoms, and taken together with the atoms connecting them, form a 5-membered heteroaromatic ring containing 1 or 2 heteroatoms independently selected from O, N, and S. In some embodiments, R⁶ and R⁷ are on adjacent atoms, and taken together with the atoms connecting them, form a 6-membered heterocyclic ring containing 1 or 2 heteroatoms independently selected from O, N, and S, optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹. In some embodiments, R⁶ and R⁷ are on adjacent atoms, and taken together with the atoms connecting them, form a 6-membered aliphatic heterocyclic ring containing 1 or 2 heteroatoms independently selected from O, N, and S. In some embodiments, R⁶ and R⁷ are on adjacent atoms, and taken together with the atoms connecting them, form a 6-membered heteroaromatic ring containing 1 or 2 heteroatoms independently selected from O, N, and S. In some embodiments, one R⁶ and one R⁷ are on adjacent atoms, and taken together with the atoms connecting them, form a C₄-C₈ carbocyclic ring or a 5- to 8-membered heterocyclic ring containing 1 or 2 heteroatoms independently selected from O, N, and S, wherein the ring is fused to the B ring at the 2- and 3-positions relative to the bond connecting the B ring to the CR⁴R⁵ group. In some embodiments, o=2; p=2 or 3; and two pairs, each of one R⁶ and one R⁷, are on adjacent atoms, and each pair of one R⁶ and one R⁷ taken together with the atoms connecting them form a C₅ carbocyclic ring optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹. In some embodiments, o=2; p=2 or 3; and two pairs, each of one R⁶ and one R⁷, are on adjacent atoms, and each pair of one R⁶ and one R⁷ taken together with the atoms connecting them form a C₅ aliphatic carbocyclic ring. In some embodiments, o=2; p=2 or 3; and two pairs, each of one R⁶ and one R⁷, are on adjacent atoms, and each pair of one R⁶ and one R⁷ taken together with the atoms connecting them form a C₆ carbocyclic ring optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹. In some embodiments, o=2; p=2 or 3; and two pairs, each of one R⁶ and one R⁷, are on adjacent atoms, and each pair of one R⁶ and one R⁷ taken together with the atoms connecting them form a C₆ aliphatic carbocyclic ring. In some embodiments, o=2; p=2 or 3; and two pairs, each of one R⁶ and one R⁷, are on adjacent atoms, and each pair of one R⁶ and one R⁷ taken together with the atoms connecting them form a C₆ aromatic carbocyclic ring. In some embodiments, o=2; p=2 or 3; and two pairs, each of one R⁶ and one R⁷, are on adjacent atoms, and each pair of one R⁶ and one R⁷ taken together with the atoms connecting them form a 5-membered heterocyclic ring containing 1 or 2 heteroatoms independently selected from O, N, and S, optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹. In some embodiments, o=2; p=2 or 3; and two pairs, each of one R⁶ and one R⁷, are on adjacent atoms, and each pair of one R⁶ and one R⁷ taken together with the atoms connecting them form a 5-membered aliphatic heterocyclic ring containing 1 or 2 heteroatoms independently selected from O, N, and S. In some embodiments, o=2; p=2 or 3; and two pairs, each of one R⁶ and one R⁷, are on adjacent atoms, and each pair of one R⁶ and one R⁷ taken together with the atoms connecting them form a 5-membered heteroaromatic ring containing 1 or 2 heteroatoms independently selected from O, N, and S. In some embodiments, o=2; p=2 or 3; and two pairs, each of one R⁶ and one R⁷, are on adjacent atoms, and each pair of one R⁶ and one R⁷ taken together with the atoms connecting them form a 6-membered heterocyclic ring containing 1 or 2 heteroatoms independently selected from O, N, and S, optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹. In some embodiments, o=2; p=2 or 3; and two pairs, each of one R⁶ and one R⁷, are on adjacent atoms, and each pair of one R⁶ and one R⁷ taken together with the atoms connecting them form a 6-membered aliphatic heterocyclic ring containing 1 or 2 heteroatoms independently selected from O, N, and S. In some embodiments, o=2; p=2 or 3; and two pairs, each of one R⁶ and one R⁷, are on adjacent atoms, and each pair of one R⁶ and one R⁷ taken together with the atoms connecting them form a 6-membered heteroaromatic ring containing 1 or 2 heteroatoms independently selected from O, N, and S. In some embodiments, o=2; p=2 or 3; and two pairs, each of one R⁶ and one R⁷, are on adjacent atoms, and each pair of one R⁶ and one R⁷ taken together with the atoms connecting them independently form a C₄-C₈ carbocyclic ring or a 5- to 8-membered heterocyclic ring containing 1 or 2 heteroatoms independently selected from O, N, and S, wherein one of the two rings is fused to the B ring at the 2- and 3-positions relative to the bond connecting the B ring to the CR⁴R⁵ group, and the other of the two rings is fused to the B ring at the 5- and 6-positions relative to the bond connecting the B ring to the CR⁴R⁵ group. In some embodiments, o=2; p=2; and two pairs, each of one R⁶ and one R⁷, are on adjacent atoms, and each pair of one R⁶ and one R⁷ taken together with the atoms connecting them form a C₅ aliphatic carbocyclic ring. In some embodiments, o=2; p=3; and two pairs, each of one R⁶ and one R⁷, are on adjacent atoms, and each pair of one R⁶ and one R⁷ taken together with the atoms connecting them form a C₅ aliphatic carbocyclic ring; and one R⁷ is halo (e.g., Cl or F). In some embodiments, o=2; p=3; and two pairs, each of one R⁶ and one R⁷, are on adjacent atoms, and each pair of one R⁶ and one R⁷ taken together with the atoms connecting them form a C₅ aliphatic carbocyclic ring; and one R⁷ is CN. In some embodiments, one R⁷ is pyrazolyl and is para to the bond connecting the B ring to the CR⁴R⁵ group of Formula AA. In some embodiments, one R⁷ is 3-pyrazolyl and is para to the bond connecting the B ring to the CR⁴R⁵ group of Formula AA. In some embodiments, one R⁷ is 4-pyrazolyl and is para to the bond connecting the B ring to the CR⁴R⁵ group of Formula AA. In some embodiments, one R⁷ is 5-pyrazolyl and is para to the bond connecting the B ring to the CR⁴R⁵ group of Formula AA. In some embodiments, one R⁷ is thiazolyl and is para to the bond connecting the B ring to the CR⁴R⁵ group of Formula AA. In some embodiments, one R⁷ is 4-thiazolyl and is para to the bond connecting the B ring to the CR⁴R⁵ group of Formula AA. In some embodiments, one R⁷ is 5-thiazolyl and is para to the bond connecting the B ring to the CR⁴R⁵ group of Formula AA. In some embodiments, one R⁷ is furyl and is para to the bond connecting the B ring to the CR⁴R⁵ group of Formula AA. In some embodiments, one R⁷ is 2-furyl and is para to the bond connecting the B ring to the CR⁴R⁵ group of Formula AA. In some embodiments, one R⁷ is thiophenyl and is para to the bond connecting the B ring to the CR⁴R⁵ group of Formula AA. In some embodiments, one R⁷ is 2-thiophenyl and is para to the bond connecting the B ring to the CR⁴R⁵ group of Formula AA. In some embodiments, one R⁷ is phenyl and is para to the bond connecting the B ring to the CR⁴R⁵ group of Formula AA. In some embodiments, one R⁷ is cycloalkenyl (e.g., cyclopentenyl, e.g., 1-cyclopentenyl) and is para to the bond connecting the B ring to the CR⁴R⁵ group of Formula AA. In some embodiments, one R⁷ is phenyl optionally substituted with one or more C₁-C₆ alkyl (e.g., methyl or propyl, e.g., 2-propyl) optionally substituted with one or more hydroxyl, NR⁸R⁹ (e.g., dimethylamino), or C₆-C₁₀ aryl (e.g., phenyl, naphthyl, or methylenedioxyphenyl) and is para to the bond connecting the B ring to the CR⁴R⁵ group of Formula AA. In some embodiments, one R⁷ is phenyl optionally substituted with one or more C₁-C₆ alkoxy (e.g., methoxy) optionally substituted with one or more hydroxyl, NR⁸R⁹ (e.g., dimethylamino), or C₆-C₁₀ aryl (e.g., phenyl, naphthyl, or methylenedioxyphenyl) and is para to the bond connecting the B ring to the CR⁴R⁵ group of Formula AA. In some embodiments, one R⁷ is phenyl optionally substituted with one or more C₆-C₁₀ aryloxy (e.g., phenoxy) and is para to the bond connecting the B ring to the CR⁴R⁵ group of Formula AA. In some embodiments, one R⁷ is phenyl optionally substituted with one or more CN and is para to the bond connecting the B ring to the CR⁴R⁵ group of Formula AA. In some embodiments, one R⁷ is phenyl optionally substituted with one or more halo (e.g., F, Cl) and is para to the bond connecting the B ring to the CR⁴R⁵ group of Formula AA and is para to the bond connecting the B ring to the CR⁴R⁵ group of Formula AA. In some embodiments, one R⁷ is phenyl optionally substituted with one or more COOC₁-C₆ alkyl (e.g., CO₂t-Bu) and is para to the bond connecting the B ring to the CR⁴R⁵ group of Formula AA. In some embodiments, one R⁷ is phenyl optionally substituted with one or more S(O₂)C₁-C₆ alkyl (e.g., S(O₂)methyl) and is para to the bond connecting the B ring to the CR⁴R⁵ group of Formula AA. In some embodiments, one R⁷ is phenyl optionally substituted with one or more 3- to 7-membered heterocycloalkyl (e.g., morpholinyl) and is para to the bond connecting the B ring to the CR⁴R⁵ group of Formula AA. In some embodiments, one R⁷ is phenyl optionally substituted with one or more CONR⁸R⁹ (e.g., unsubstituted amido) and is para to the bond connecting the B ring to the CR⁴R⁵ group of Formula AA. In some embodiments, one R⁷ is phenyl optionally substituted with one or more C₁-C₆ alkyl (e.g., methyl or propyl, e.g., 2-propyl) and with one or more halo (e.g., F, Cl) and is para to the bond connecting the B ring to the CR⁴R⁵ group of Formula AA and is para to the bond connecting the B ring to the CR⁴R⁵ group of Formula AA.

In some embodiments, R⁶ and R⁷ are each attached to a carbon of an aryl ring B. In some embodiments, R⁶ and R⁷ are each attached to a carbon of a heteroaryl ring B. In some embodiments, R⁶ is attached to a carbon and R⁷ is attached to a nitrogen of a heteroaryl ring B. In some embodiments, R⁷ is attached to a carbon and R⁶ is attached to a nitrogen of a heteroaryl ring B.

In some embodiments, the optionally substituted ring B is

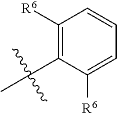

and each R⁶ is independently selected from the group consisting of: C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl, wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆, alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl.

In some embodiments, the optionally substituted ring B is

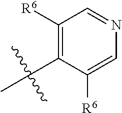

and each R⁶ is independently selected from the group consisting of: C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl, wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆, alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl.

In some embodiments, the optionally substituted ring B is

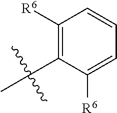

and each R⁶ is independently selected from the group consisting of: C₁-C₆ alkyl, C₃-C₇ cycloalkyl, C₁-C₆ haloalkyl, C₁-C₆ alkoxy, C₁-C₆ haloalkoxy, wherein the C₁-C₆ alkyl, C₁-C₆ haloalkyl, and C₃-C₇ cycloalkyl is optionally substituted with one or more substituents each independently selected from hydroxy, halo, CN, or oxo.

In some embodiments, the optionally substituted ring B is

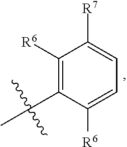

wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
   or R⁶ and R⁷, taken together with the atoms connecting them, independently form C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.
   In some embodiments, the optionally substituted ring B is

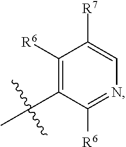

wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
   or R⁶ and R⁷, taken together with the atoms connecting them, independently form C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, the optionally substituted ring B is

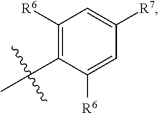

wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy.
In some embodiments, the optionally substituted ring B is

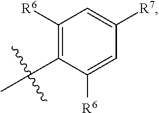

R⁶, Wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;

wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy, and wherein R⁷ is optionally fused to a five-to-seven-membered carbocyclic ring or heterocyclic ring containing one or two heteroatoms independently selected from oxygen, sulfur and nitrogen.

In some embodiments, the optionally substituted ring B is

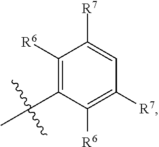

wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
   or at least one pair of R⁶ and R⁷ on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, the optionally substituted ring B is

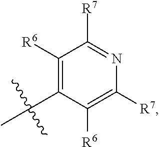

wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
   or at least one pair of R⁶ and R⁷ on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, the optionally substituted ring B is

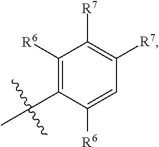

wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
or R⁶ and R⁷, taken together with the atoms connecting them, independently form a C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, the optionally substituted ring B is

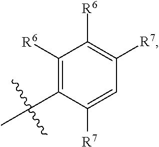

wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
or R⁶ and R⁷, taken together with the atoms connecting them, independently form a C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, the optionally substituted ring B is

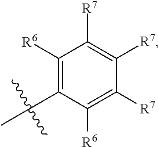

wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
or at least one pair of R⁶ and R⁷ on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.
   The Groups R^{6′} and R^{7′}

In some embodiments,

R⁶′ and R⁷′ are each independently selected from C₁-C₆ alkyl, C₁-C₆ haloalkyl, C₁-C₆ alkoxy, C₁-C₆ haloalkoxy, Cl, Br, I, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₁₀ cycloalkyl and 3- to 10-membered heterocycloalkyl, and a C₂-C₆ alkenyl,
wherein R⁶′ and R⁷′ are each optionally substituted with one or more substituents independently selected from
hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl,
C₆-C₁₀ aryloxy, and S(O₂)C₁-C₆ alkyl; and wherein the C₁-C₆ alkyl or C₁-C₆ alkoxy that R⁶′ or R⁷′ is substituted with is optionally substituted with one or more hydroxyl, halo, C₆-C₁₀ aryl or NR⁸R⁹, or wherein R⁶′ or R⁷′ is optionally fused to a five-to-seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or at least one pair of R⁶′ and R⁷′ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments,

R⁶′ and R⁷′ are each independently selected from C₁-C₆ alkyl, C₁-C₆ haloalkyl, C₁-C₆, alkoxy, C₁-C₆ haloalkoxy, Cl, Br, I, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₇ cycloalkyl and 3- to 7-membered heterocycloalkyl,
wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or at least one pair of R⁶′ and R⁷′ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments,

R⁶′ and R⁷′ are each independently selected from C₁-C₆ haloalkyl, C₁-C₆ alkoxy, C₁-C₆, haloalkoxy, Cl, Br, I, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₇ cycloalkyl and 3- to 7-membered heterocycloalkyl,
wherein the C₃-C₇ cycloalkyl, C₁-C₆ haloalkyl, and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or at least one pair of R⁶′ and R⁷′ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments,

R⁶′ and R⁷′ are each independently selected from C₁-C₆ alkyl, Cl, Br, I, NO₂, COC₁-C₆ alkyl, CO₂C₁-C₆ alkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₇ cycloalkyl and 3- to 7-membered heterocycloalkyl,
wherein the C₁-C₆ alkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or at least one pair of R⁶′ and R⁷′ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments,

R⁶′ and R⁷′ are each independently selected from C₁-C₆ alkyl, C₁-C₆ haloalkyl, C₁-C₆, alkoxy, C₁-C₆ haloalkoxy, Cl, Br, I, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₇ cycloalkyl and 3- to 7-membered heterocycloalkyl,
wherein the C₁-C₆ alkyl, C₁-C₆ haloalkyl, C₃-C₇ cycloalkyl, and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are unsubstituted;
   or at least one pair of R⁶′ and R⁷′ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments,

R⁶′ and R⁷′ are each independently selected from C₁-C₆ alkyl, C₁-C₆ haloalkyl, C₁-C₆ alkoxy, C₁-C₆ haloalkoxy, Cl, Br, I, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₇ cycloalkyl and 3- to 7-membered heterocycloalkyl,
wherein the C₁-C₆ alkyl, C₃-C₇ cycloalkyl and 3- to 7-membered heterocycloalkyl are each unsubstituted;
or at least one pair of R⁶′ and R⁷′ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments,

R⁶′ is independently selected from C₁-C₆ alkyl, C₃-C₇ cycloalkyl, C₁-C₆ haloalkyl, C₁-C₆ alkoxy, C₁-C₆ haloalkoxy, Cl, Br, I, C₆-C₁₀ aryl, 5- to 10-membered heteroaryl, CO—C₁-C₆ alkyl; CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
and R⁷′ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
   or R⁶′ and R⁷′, taken together with the atoms connecting them, independently form C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments,

R⁶′ and R⁷′ are each independently selected from C₁-C₆ alkyl, C₁-C₆ alkoxy, Cl, Br, I, NO₂, COC₁-C₆ alkyl, CO₂C₁-C₆ alkyl, C₆-C₁₀ aryl, 5- to 10-membered heteroaryl, CONR⁸R⁹, and 3- to 7-membered heterocycloalkyl,
wherein the C₁-C₆ alkyl and 3- to 7-membered heterocycloalkyl is optionally substituted with one or more substituents each independently selected from hydroxy or oxo, or at least one pair of R⁶′ and R⁷′ on adjacent atoms, taken together with the atoms connecting them, independently form at least one C₄-C₈ carbocyclic ring, wherein the carbocyclic ring is optionally independently substituted with one or more hydroxy or oxo.

In some embodiments, at least one pair of R⁶′ and R⁷′ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments, R⁶′ and R⁷′ are each independently selected from C₁-C₆ alkyl, 5- to 10-membered heteroaryl, and 3- to 7-membered heterocycloalkyl;
wherein the C₁-C₆ alkyl is optionally substituted with one or more substituents each independently selected from hydroxyl or C₁-C₆ alkoxy.

In some embodiments, R⁶′ is C₁-C₆ alkyl. In some embodiments, R⁶′ is isopropyl. In some embodiments, R⁶′ is n-propyl. In some embodiments, R⁶ is butyl (e.g., s-butyl, iso-butyl). In some embodiments, R⁶′ is C₃-C₁₀ cycloalkyl. In some embodiments, R⁶′ is cyclopropyl. In some embodiments, R⁶′ is Cl, Br, or I. In some embodiments, R⁶′ is CN. In some embodiments, R⁶′ is C₁-C₆ alkyl substituted with hydroxyl (e.g., hydroxymethyl, hydroxyethyl, or 2-hydroxy-2-propyl. In some embodiments, R⁶′ is C₁-C₆ alkyl substituted with C₁-C₆ alkoxy (e.g., methoxymethyl. In some embodiments, R⁶′ is C₁-C₆ alkyl substituted with O(C₃-C₁₀ cycloalkyl)

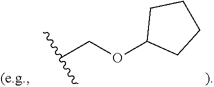 [SMILES: *COC1CCCC1]

In some embodiments, R⁶′ is C₆-C₁₀ aryl, optionally fused to a five-to-seven-membered carbocyclic ring or heterocyclic ring containing one or two heteroatoms independently selected from oxygen, sulfur and nitrogen. In some embodiments, R⁶′ is phenyl, optionally fused to a five-to-seven-membered carbocyclic ring or heterocyclic ring containing one or two heteroatoms independently selected from oxygen, sulfur and nitrogen. For example, R⁶′ is

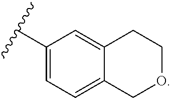 [SMILES: *c1ccc2c(c1)CCOC2]

In some embodiments, R⁶′ is imidazolyl. In some embodiments, R⁶′ is pyrazolyl. In some embodiments, R⁶′ is pyrrolyl. In some embodiments, R⁶′ is thiazolyl. In some embodiments, R⁶′ is isothiazolyl. In some embodiments, R⁶′ is oxazolyl. In some embodiments, R⁶′ is isoxazolyl. In some embodiments, R⁶′ is pyridyl. In some embodiments, R⁶′ is pyrimidinyl. In some embodiments, R⁷′ is C₁-C₆ alkyl. In some embodiments, R⁷′ is isopropyl. In some embodiments, R⁷′ is n-propyl. In some embodiments, R⁷ is butyl (e.g., s-butyl, iso-butyl). In some embodiments, R⁷′ is C₃-C₁₀ cycloalkyl. In some embodiments, R⁷′ is cyclopropyl. In some embodiments, R⁷′ is Cl, Br, or I. In some embodiments, R⁷′ is CN. In some embodiments, R⁷′ is C₁-C₆ alkyl substituted with hydroxyl (e.g., hydroxymethyl, hydroxyethyl, or 2-hydroxy-2-propyl. In some embodiments, R⁷′ is C₁-C₆, alkyl substituted with C₁-C₆ alkoxy (e.g., methoxymethyl). In some embodiments, R⁷′ is C₁-C₆ alkyl substituted with O(C₃-C₁₀ cycloalkyl)

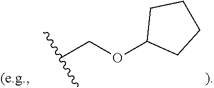 [SMILES: *COC1CCCC1]

In some embodiments, R⁷′ is C₆-C₁₀ aryl, optionally fused to a five-to-seven-membered carbocyclic ring or heterocyclic ring containing one or two heteroatoms independently selected from oxygen, sulfur and nitrogen. In some embodiments, R⁷′ is phenyl, optionally fused to a five-to-seven-membered carbocyclic ring or heterocyclic ring containing one or two heteroatoms independently selected from oxygen, sulfur and nitrogen. For example, R⁷′ is

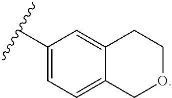 [SMILES: *c1ccc2c(c1)CCOC2]

In some embodiments, R⁷′ is imidazolyl. In some embodiments, R⁷′ is pyrazolyl. In some embodiments, R⁷′ is pyrrolyl. In some embodiments, R⁷′ is thiazolyl. In some embodiments, R⁷′ is isothiazolyl. In some embodiments, R⁷′ is oxazolyl. In some embodiments, R⁷′ is isoxazolyl. In some embodiments, R⁷′ is pyridyl. In some embodiments, R⁷′ is pyrimidinyl.

In some embodiments, o=1; p=0; and

R⁶′ is selected from C₁-C₆ alkyl, C₁-C₆ haloalkyl, C₁-C₆ alkoxy, C₁-C₆ haloalkoxy, Cl, Br, I, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₇ cycloalkyl and 3- to 7-membered heterocycloalkyl, wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl.

In some embodiments, o=1; p=0; and

R⁶′ is selected from C₁-C₆ alkyl, C₁-C₆ alkoxy, Cl, Br, I, NO₂, COC₁-C₆ alkyl, CO₂C₁-C₆ alkyl, C₆-C₁₀ aryl, 5- to 10-membered heteroaryl, CONR⁸R⁹, and 3- to 7-membered heterocycloalkyl, wherein the C₁-C₆ alkyl and 3- to 7-membered heterocycloalkyl is optionally substituted with one or more substituents each independently selected from hydroxy or oxo.

In some embodiments, o=1 or 2; p=1, 2, or 3; and

R⁶′ and R⁷′ are each independently selected from C₁-C₆ alkyl, C₁-C₆ haloalkyl, C₁-C₆, alkoxy, C₁-C₆ haloalkoxy, Cl, Br, I, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₇ cycloalkyl and 3- to 7-membered heterocycloalkyl,
wherein the C₁-C₆ alkyl, C₁-C₆ haloalkyl, C₃-C₇ cycloalkyl, and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl.

In some embodiments, o=2; p=1; and

each R⁶′ is independently selected from C₁-C₆ alkyl, C₃-C₇ cycloalkyl, C₁-C₆ haloalkyl, C₁-C₆ alkoxy, C₁-C₆ haloalkoxy, Cl, Br, I, C₆-C₁₀ aryl, 5- to 10-membered heteroaryl, CO—C₁-C₆ alkyl; CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
and R⁷′ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
   or R⁶′ and R⁷′, taken together with the atoms connecting them, independently form C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2 or 3; and

each R⁶′ is independently selected from C₁-C₆ alkyl, C₃-C₇ cycloalkyl, C₁-C₆ haloalkyl, C₁-C₆ alkoxy, C₁-C₆ haloalkoxy, Cl, Br, I, C₆-C₁₀ aryl, 5- to 10-membered heteroaryl, CO—C₁-C₆ alkyl; CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein each R⁷′ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
   or at least one pair of R⁶′ and R⁷′ on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments, o=1 or 2; p=1, 2, or 3; and

R⁶′ and R⁷′ are each independently selected from C₁-C₆ alkyl, C₁-C₆ alkoxy, Cl, Br, I, NO₂, COC₁-C₆ alkyl, CO₂C₁-C₆ alkyl, C₆-C₁₀ aryl, 5- to 10-membered heteroaryl, CONR⁸R⁹, and 3- to 7-membered heterocycloalkyl,
wherein the C₁-C₆ alkyl and 3- to 7-membered heterocycloalkyl is optionally substituted with one or more substituents each independently selected from hydroxy or oxo, or at least one pair of R⁶′ and R⁷′ on adjacent atoms, taken together with the atoms connecting them, independently form at least one C₄-C₈ carbocyclic ring, wherein the carbocyclic ring is optionally independently substituted with one or more hydroxy or oxo.

In some embodiments, o=1 or 2; p=1, 2, or 3; and

R⁶′ and R⁷′ are each independently selected from C₁-C₆ alkyl, C₁-C₆ alkoxy, Cl, Br, I, NO₂, COC₁-C₆ alkyl, CO₂C₁-C₆ alkyl, C₆-C₁₀ aryl, 5- to 10-membered heteroaryl, CONR⁸R⁹, and 3- to 7-membered heterocycloalkyl, wherein the C₁-C₆ alkyl and 3- to 7-membered heterocycloalkyl is optionally substituted with one or more substituents each independently selected from hydroxy or oxo.

In some embodiments, o=1 or 2; p=1, 2, or 3; and

one R⁶′ and one R⁷′ are on adjacent atoms, and taken together with the atoms connecting them, form a C₄-C₈ carbocyclic ring or a 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=1 or 2; p=1, 2, or 3; and

one R⁶′ and one R⁷′ are on adjacent atoms, and taken together with the atoms connecting them, form a C₆ carbocyclic ring or a 5-to-6-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=1 or 2; p=1, 2, or 3; and

one R⁶′ and one R⁷′ are on adjacent atoms, and taken together with the atoms connecting them, form a C₄-C₈ carbocyclic ring or a 5- to 8-membered heterocyclic ring containing 1 or 2 heteroatoms independently selected from O, N, and S, wherein the carbocyclic ring or heterocyclic ring is unsubstituted.

In some embodiments, o=2; p=2 or 3; and

two pairs, each of one R⁶′ and one R⁷′, are on adjacent atoms, and each pair of one R⁶′ and one R⁷′ taken together with the atoms connecting them independently form a C₄-C₈ carbocyclic ring or a 5- to 8-membered heterocyclic ring containing 1 or 2 heteroatoms independently selected from O, N, and S, wherein each carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2 or 3; and

two pairs, each of one R⁶′ and one R⁷′, are on adjacent atoms, and each pair of one R⁶′ and one R⁷′ taken together with the atoms connecting them independently form a C₆ carbocyclic ring or a 5-to-6-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2 or 3; and

two pairs, each of one R⁶′ and one R⁷′, are on adjacent atoms, and each pair of one R⁶′ and one R⁷′ taken together with the atoms connecting them independently form a C₄-C₈ carbocyclic ring or a 5- to 8-membered heterocyclic ring containing 1 or 2 heteroatoms independently selected from O, N, and S, wherein the carbocyclic ring or heterocyclic ring is unsubstituted.

### Particular Embodiments Wherein o=1; p=0

In some embodiments, R⁶′ is C₁-C₆ alkyl. In some embodiments, R⁶′ is isopropyl. In some embodiments, R⁶ is n-propyl. In some embodiments, R⁶ is butyl (e.g., sec-butyl, iso-butyl). In some embodiments, R⁶′ is ethyl. In some embodiments, R⁶′ is methyl. In some embodiments, R⁶′ is C₁-C₆ alkyl substituted with one or more halo. In some embodiments, R⁶′ is trifluoromethyl. In some embodiments, R⁶′ is trifluoromethoxy. In some embodiments, R⁶′ is C₃-C₇ cycloalkyl. In some embodiments, R⁶′ is cyclopropyl. In some embodiments, R⁶′ is Cl, Br, or I, In some embodiments, R⁶′ is chloro. In some embodiments, R⁶′ is attached to a carbon of an aryl ring B. In some embodiments, R⁶′ is attached to a carbon of a heteroaryl ring B. In some embodiments, R⁶′ is attached to a nitrogen of a heteroaryl ring B.

### Particular Embodiments Wherein o=1 or 2; p=1, 2. Or 3

In some embodiments, at least one R⁶′ is C₁-C₆ alkyl, and at least one R⁷′ is C₁-C₆ alkyl optionally substituted with one or more halo. In some embodiments, at least one R⁶′ is C₁-C₆, alkyl and at least one R⁷′ is C₁-C₆ alkyl. In some embodiments, at least one R⁶′ is isopropyl and at least one R⁷′ is methyl. In some embodiments, at least one R^{6′} is isopropyl and at least one R^{7′} is isopropyl. In some embodiments, at least one R^{6′} is isopropyl and at least one R^{7′} is n-propyl. In some embodiments, at least one R^{6′} is isopropyl and at least one R^{7′} is s-butyl. In some embodiments, at least one R^{6′} is isopropyl and at least one R^{7′} is iso-butyl. In some embodiments, o=1; p=1; R^{6′} is isopropyl; and R^{7′} is isopropyl. In some embodiments, o=2; p=1, 2, or 3; one R^{6′} is isopropyl; and one R^{6′} is isopropyl. In some embodiments, o=2; p=1, 2, or 3; one R^{6′} is isopropyl; and one R^{7′} is n-propyl. In some embodiments, o=2; p=1, 2, or 3; one R^{6′} is isopropyl; and one R^{7′} is iso-butyl. In some embodiments, o=2; p=1, 2, or 3; one R^{6′} is isopropyl; and one R^{7′} is sec-butyl. In certain of the foregoing embodiments (when o=2; p=1, 2, or 3; one R^{6′} is isopropyl; and one R^{7′} is isopropyl, n-propyl, iso-butyl, or sec-butyl), the other R^{6′} is cyano.

In certain of the foregoing embodiments (when o=2; p=1, 2, or 3; one R^{6′} is isopropyl; and one R^{7′} is isopropyl, n-propyl, iso-butyl, or sec-butyl), the other R⁶ is Cl, Br, or I.

In some embodiments, at least one R⁶′ is C₁-C₆ alkyl, and at least one R⁷′ is C₁-C₆ alkyl substituted with one or more halo.

In some embodiments, at least one R⁶′ is isopropyl and at least one R⁷′ is trifluoromethyl. In some embodiments, at least one R⁶′ is C₁-C₆ alkyl, and at least one R⁷′ is C₃-C₇ cycloalkyl. In some embodiments, at least one R⁶′ is isopropyl and at least one R⁷′ is cyclopropyl. In some embodiments, o=1; p=1; R⁶′ is isopropyl; and R⁷′ is cyclopropyl. In some embodiments, o=2; p=1, 2, or 3; one R^{6′} is isopropyl; and one R^{7′} is cyclopropyl. In some embodiments, o=2; p=1, 2, or 3; one R^{6′} is isopropyl; and one R^{7′} is cyclopropyl. In some embodiments, o=2; p=1, 2, or 3; one R^{6′} is isopropyl; and one R^{7′} is cyclopropyl. In certain of the foregoing embodiments (when o=2; p=1, 2, or 3; one R^{6′} is isopropyl; and one R^{7′} is cyclopropyl), the other R^{6′} is halo. In some embodiments, at least one R⁶′ is C₁-C₆ alkyl, and at least one R⁷′ is Cl, Br, or I, In some embodiments, at least one R⁶′ is isopropyl and at least one R⁷′ is Cl, Br, or I, In some embodiments, at least one R⁶′ is isopropyl and at least one R⁷′ is chloro. In some embodiments, o=1; p=1; R⁶′ is isopropyl; and R⁷′ is chloro. In some embodiments, o=2; p=1; at least one R^{6′} is isopropyl; and R⁷′ is chloro. In some embodiments, o=2; p=1; one R⁶′ is isopropyl; the other R⁶′ is trifluoromethyl; and R⁷′ is chloro. In some embodiments, at least one R⁶′ is C₃-C₇ cycloalkyl, and at least one R⁷′ is C₃-C₇ cycloalkyl. In some embodiments, at least one R⁶′ is cyclopropyl, and at least one R⁷′ is cyclopropyl. In some embodiments, at least one R⁶′ is C₃-C₇ cycloalkyl, and at least one R⁷′ is Cl, Br, or I, In some embodiments, at least one R⁶′ is cyclopropyl and at least one R⁷′ is Cl, Br, or I, In some embodiments, at least one R⁶′ is cyclopropyl and at least one R⁷′ is chloro. In some embodiments, o=1; p=1; R⁶′ is cyclopropyl; and R⁷′ is chloro. In some embodiments, at least one R⁶′ is C₁-C₆ alkyl, and at least one R⁷′ is C₁-C₆ alkoxy optionally substituted with one or more halo. In some embodiments, at least one R⁶′ is isopropyl, and at least one R⁷′ is C₁-C₆ alkoxy. In some embodiments, at least one R⁶′ is isopropyl, and at least one R⁷′ is methoxy. In some embodiments, o=1; p=1; R⁶′ is isopropyl, and R⁷′ is methoxy. In some embodiments, o=2; p=1; at least one R⁶′ is isopropyl, and R⁷′ is methoxy. In some embodiments, at least one R⁶′ is C₁-C₆ alkyl, and at least one R⁷′ is C₁-C₆ alkoxy substituted with one or more halo. In some embodiments, at least one R⁶′ is isopropyl, and at least one R⁷′ is trifluoromethoxy. In some embodiments, at least one R⁶′ is isopropyl, and at least one R⁷′ is difluoromethoxy. In some embodiments, at least one R⁶′ is Cl, Br, or I, and at least one R⁷′ is C₁-C₆ haloalkyl optionally substituted with hydroxy. In some embodiments, at least one R⁶′ is C₁-C₆ alkyl, and at least one R⁷′ is C₁-C₆ alkyl which is optionally substituted with O(C₃-C₁₀ cycloalkyl). In some embodiments, at least one R⁶′ is isopropyl, and at least one R⁷′ is C₁-C₆ alkyl which is optionally substituted with O(C₃-C₁₀ cycloalkyl). In some embodiments, at least one R⁶′ is isopropyl, and at least one R⁷′ is

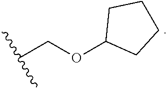 [SMILES: *COC1CCCC1]

In some embodiments, at least one R^{6′} is C₁-C₆ alkyl, and at least one R⁷′ is C₁-C₆ alkyl which is optionally substituted with C₁-C₆ alkoxy. In some embodiments, at least one R^{6′} is isopropyl, and at least one R^{7′} is C₁-C₆ alkyl which is optionally substituted with C₁-C₆ alkoxy. In some embodiments, at least one R^{6′} is isopropyl, and at least one R^{7′} is C₁-C₆ alkyl which is optionally substituted with methoxy. In some embodiments, at least one R^{6′} is isopropyl, and at least one R^{7′} is C₁-C₆ alkyl which is optionally substituted with methoxymethyl.

In some embodiments, o=1; p=1; R⁶′ is chloro, and R⁷′ is trifluoromethyl. In some embodiments, at least one R⁶′ is Cl, Br, or I, and at least one R⁷′ is C₁-C₆ haloalkoxy. In some embodiments, at least one R⁶′ is chloro, and at least one R⁷′ is trifluoromethoxy. In some embodiments, o=1; p=1; R⁶′ is chloro, and R⁷′ is trifluoromethoxy. In some embodiments, at least one R⁶′ is C₁-C₆ alkoxy; and at least one R⁷′ is Cl, Br, or I, In some embodiments, o=1; p=2; R⁶′ is C₁-C₆ alkoxy; and at least one R⁷′ is chloro.

In some embodiments, at least one R⁶ is C₁-C₆ alkyl, and at least one R⁷ is C₆-C₁₀ aryl, optionally optionally fused to a five-to-seven-membered carbocyclic ring or heterocyclic ring containing one or two heteroatoms independently selected from oxygen, sulfur and nitrogen. In some embodiments, at least one R⁶ is isopropyl, and at least one R⁷ is C₆-C₁₀ aryl, optionally optionally fused to a five-to-seven-membered carbocyclic ring or heterocyclic ring containing one or two heteroatoms independently selected from oxygen, sulfur and nitrogen.

In some embodiments, at least one R^{6′} is isopropyl, and at least one R^{7′} is

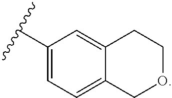 [SMILES: *c1ccc2c(c1)CCOC2]

In some embodiments, at least one R⁷′ is C₁-C₆ alkyl, and at least one R⁶′ is C₁-C₆ alkyl optionally substituted with one or more halo. In some embodiments, at least one R⁷′ is isopropyl and at least one R⁶′ is methyl. In some embodiments, at least one R^{7′} is isopropyl and at least one R^{6′} is isopropyl. In some embodiments, at least one R^{7′} is isopropyl and at least one R^{6′} is s-butyl. In some embodiments, at least one R^{7′} is isopropyl and at least one R^{6′} is iso-butyl. In some embodiments, o=1; p=1; R^{7′} is isopropyl; and R^{6′} is isopropyl. In some embodiments, o=2; p=1, 2, or 3; one R^{7′} is isopropyl; and one R^{6′} is isopropyl. In some embodiments, o=2; p=1, 2, or 3; one R^{7′} is isopropyl; and one R^{6′} is n-propyl. In some embodiments, o=2; p=1, 2, or 3; one R^{7′} is isopropyl; and one R^{6′} is iso-butyl. In some embodiments, o=2; p=1, 2, or 3; one R^{7′} is isopropyl; and one R^{6′} is sec-butyl. In certain of the foregoing embodiments (when o=2; p=1, 2, or 3; one R^{7′} is isopropyl; and one R^{6′} is isopropyl, n-propyl, iso-butyl, or sec-butyl), the other R^{6′} is cyano. In certain of the foregoing embodiments (when o=2; p=1, 2, or 3; one R^{7′} is isopropyl; and one R^{6′} is isopropyl, n-propyl, iso-butyl, or sec-butyl), the other R^{6′} is halo. In some embodiments, at least one R⁷′ is C₁-C₆ alkyl, and at least one R⁶′ is C₁-C₆ alkyl substituted with one or more halo. In some embodiments, at least one R⁷′ is isopropyl and at least one R⁶′ is trifluoromethyl. In some embodiments, at least one R⁷′ is C₁-C₆ alkyl, and at least one R⁶′ is C₃-C₇ cycloalkyl. In some embodiments, at least one R⁷′ is isopropyl and at least one R⁶′ is cyclopropyl. In some embodiments, o=1; p=1; R⁷′ is isopropyl; and R⁶′ is cyclopropyl. In some embodiments, o=2; p=1, 2, or 3; one R^{7′} is isopropyl; and one R^{6′} is cyclopropyl. In some embodiments, o=2; p=1, 2, or 3; one R^{7′} is isopropyl; and one R^{6′} is cyclopropyl. In certain of the foregoing embodiments (when o=2; p=1, 2, or 3; one R^{7′} is isopropyl; and one R^{6′} is cyclopropyl), the other R^{6′} is halo. In some embodiments, at least one R⁷′ is C₁-C₆ alkyl, and at least one R⁶′ is Cl, Br, or I, In some embodiments, at least one R⁷′ is isopropyl and at least one R⁶′ is Cl, Br, or I, In some embodiments, at least one R⁷′ is isopropyl and at least one R⁶′ is chloro. In some embodiments, o=1; p=2; one R⁷′ is isopropyl; the other R⁷′ is trifluoromethyl; and R⁶′ is chloro. In some embodiments, at least one R⁷′ is C₃-C₇ cycloalkyl, and at least one R⁶′ is C₃-C₇ cycloalkyl. In some embodiments, at least one R⁷′ is cyclopropyl, and at least one R⁶′ is cyclopropyl. In some embodiments, at least one R⁷′ is C₃-C₇ cycloalkyl, and at least one R⁶′ is Cl, Br, or I, In some embodiments, at least one R⁷′ is cyclopropyl and at least one R⁶′ is Cl, Br, or I, In some embodiments, at least one R⁷′ is cyclopropyl and at least one R⁶′ is chloro. In some embodiments, o=1; p=1; R⁷′ is cyclopropyl; and R⁶′ is chloro. In some embodiments, at least one R⁷′ is C₁-C₆ alkyl, and at least one R⁶′ is C₁-C₆ alkoxy optionally substituted with one or more halo. In some embodiments, at least one R⁷′ is isopropyl, and at least one R⁶′ is C₁-C₆ alkoxy. In some embodiments, at least one R⁷′ is isopropyl, and at least one R⁶′ is methoxy. In some embodiments, o=1; p=1; R⁷′ is isopropyl, and R⁶′ is methoxy. In some embodiments, o=2; p=1; R⁷′ is isopropyl, and at least one R⁶′ is methoxy. In some embodiments, at least one R⁷′ is C₁-C₆ alkyl, and at least one R⁶′ is C₁-C₆ alkoxy substituted with one or more halo. In some embodiments, at least one R⁷′ is isopropyl, and at least one R⁶′ is trifluoromethoxy. In some embodiments, at least one R⁷′ is Cl, Br, or I, and at least one R⁶′ is C₁-C₆ haloalkyl optionally substituted with one or more hydroxy. In some embodiments, at least one R⁷ is C₁-C₆ alkyl, and at least one R⁶ is C₁-C₆ alkyl which is optionally substituted with O(C₃-C₁₀ cycloalkyl).

In some embodiments, at least one R⁷ is isopropyl, and at least one R⁶ is C₁-C₆ alkyl which is optionally substituted with O(C₃-C₁₀ cycloalkyl) In some embodiments, at least one R⁷ is isopropyl, and at least one R^{6′} is

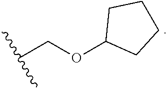 [SMILES: *COC1CCCC1]

In some embodiments, at least one R^{7′} is C₁-C₆ alkyl, and at least one R^{6′} is C₁-C₆ alkyl which is optionally substituted with C₁-C₆ alkoxy. In some embodiments, at least one R^{7′} is isopropyl, and at least one R^{6′} is C₁-C₆ alkyl which is optionally substituted with C₁-C₆ alkoxy. In some embodiments, at least one R⁷ is isopropyl, and at least one R^{6′} is C₁-C₆ alkyl which is optionally substituted with methoxy. In some embodiments, at least one R^{7′} is isopropyl, and at least one R^{6′} is C₁-C₆ alkyl which is optionally substituted with methoxymethyl. In some embodiments, o=1; p=1; R⁷′ is chloro, and R⁶′ is trifluoromethyl. In some embodiments, at least one R⁷′ is Cl, Br, or I, and at least one R⁶′ is C₁-C₆ haloalkoxy. In some embodiments, at least one R⁷′ is chloro, and at least one R⁶′ is trifluoromethoxy. In some embodiments, o=1; p=1; R⁷′ is chloro, and R⁶′ is trifluoromethoxy.

In some embodiments, at least one R⁷′ is C₁-C₆ alkoxy; and at least one R⁶′ is Cl, Br, or I, In some embodiments, o=1; p=2; at least one R⁷′ is C₁-C₆ alkoxy; and R⁶′ is chloro. In some embodiments, at least one R^{7′} is C₁-C₆ alkyl, and at least one R^{6′} is C₆-C₁₀ aryl, optionally optionally fused to a five-to-seven-membered carbocyclic ring or heterocyclic ring containing one or two heteroatoms independently selected from oxygen, sulfur and nitrogen. In some embodiments, at least one R^{7′} is isopropyl, and at least one R^{6′} is C₆-C₁₀ aryl, optionally optionally fused to a five-to-seven-membered carbocyclic ring or heterocyclic ring containing one or two heteroatoms independently selected from oxygen, sulfur and nitrogen. In some embodiments, at least one R^{7′} is isopropyl, and at least one R^{6′} is

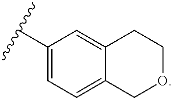 [SMILES: *c1ccc2c(c1)CCOC2]

In some embodiments, R⁶′ and R⁷′ are each attached to a carbon of an aryl ring B. In some embodiments, R⁶′ and R⁷′ are each attached to a carbon of a heteroaryl ring B. In some embodiments, R⁶′ is attached to a carbon and R⁷′ is attached to a nitrogen of a heteroaryl ring B. In some embodiments, R⁷′ is attached to a carbon and R⁶′ is attached to a nitrogen of a heteroaryl ring B.

In some embodiments, one R⁶′ and one R⁷′ are on adjacent atoms, and taken together with the atoms connecting them, form a C₅ carbocyclic ring optionally substituted with one or more substituents independently selected from hydroxy, halo, oxo, C₁-C₆ alkyl, C₁-C₆, alkoxy, NR⁸R⁹, ═NR¹⁰, COOC₁-C₆ alkyl, C₆-C₁₀ aryl, and CONR⁸R⁹. In some embodiments, R⁶′ and R⁷′ are on adjacent atoms, and taken together with the atoms connecting them, form a C₅ aliphatic carbocyclic ring. In some embodiments, R⁶′ and R⁷′ are on adjacent atoms, and taken together with the atoms connecting them, form a C₆ carbocyclic ring optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹. In some embodiments, R⁶′ and R⁷′ are on adjacent atoms, and taken together with the atoms connecting them, form a C₆ aliphatic carbocyclic ring. In some embodiments, R⁶′ and R⁷′ are on adjacent atoms, and taken together with the atoms connecting them, form a C₆ aromatic carbocyclic ring. In some embodiments, R⁶′ and R⁷′ are on adjacent atoms, and taken together with the atoms connecting them, form a 5-membered heterocyclic ring containing 1 or 2 heteroatoms independently selected from O, N, and S, optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹. In some embodiments, R⁶′ and R⁷′ are on adjacent atoms, and taken together with the atoms connecting them, form a 5-membered aliphatic heterocyclic ring containing 1 or 2 heteroatoms independently selected from O, N, and S. In some embodiments, R⁶′ and R⁷′ are on adjacent atoms, and taken together with the atoms connecting them, form a 5-membered heteroaromatic ring containing 1 or 2 heteroatoms independently selected from O, N, and S. In some embodiments, R⁶′ and R⁷′ are on adjacent atoms, and taken together with the atoms connecting them, form a 6-membered heterocyclic ring containing 1 or 2 heteroatoms independently selected from O, N, and S, optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹. In some embodiments, R⁶′ and R⁷′ are on adjacent atoms, and taken together with the atoms connecting them, form a 6-membered aliphatic heterocyclic ring containing 1 or 2 heteroatoms independently selected from O, N, and S. In some embodiments, R⁶′ and R⁷′ are on adjacent atoms, and taken together with the atoms connecting them, form a 6-membered heteroaromatic ring containing 1 or 2 heteroatoms independently selected from O, N, and S. In some embodiments, one R⁶′ and one R⁷′ are on adjacent atoms, and taken together with the atoms connecting them, form a C₄-C₈ carbocyclic ring or a 5- to 8-membered heterocyclic ring containing 1 or 2 heteroatoms independently selected from O, N, and S, wherein the ring is fused to the B ring at the 2- and 3-positions relative to the bond connecting the B ring to the CR⁴R⁵ group. In some embodiments, o=2; p=2 or 3; and two pairs, each of one R⁶′ and one R⁷′, are on adjacent atoms, and each pair of one R⁶′ and one R⁷′ taken together with the atoms connecting them form a C₅ carbocyclic ring optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹. In some embodiments, o=2; p=2 or 3; and two pairs, each of one R⁶′ and one R⁷′, are on adjacent atoms, and each pair of one R⁶′ and one R⁷′ taken together with the atoms connecting them form a C₅ aliphatic carbocyclic ring. In some embodiments, o=2; p=2 or 3; and two pairs, each of one R⁶′ and one R⁷′, are on adjacent atoms, and each pair of one R⁶′ and one R⁷′ taken together with the atoms connecting them form a C₆ carbocyclic ring optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹. In some embodiments, o=2; p=2 or 3; and two pairs, each of one R⁶′ and one R⁷′, are on adjacent atoms, and each pair of one R⁶′ and one R⁷′ taken together with the atoms connecting them form a C₆ aliphatic carbocyclic ring. In some embodiments, o=2; p=2 or 3; and two pairs, each of one R⁶′ and one R⁷′, are on adjacent atoms, and each pair of one R⁶′ and one R⁷′ taken together with the atoms connecting them form a C₆ aromatic carbocyclic ring. In some embodiments, o=2; p=2 or 3; and two pairs, each of one R⁶′ and one R⁷′, are on adjacent atoms, and each pair of one R⁶′ and one R⁷′ taken together with the atoms connecting them form a 5-membered heterocyclic ring containing 1 or 2 heteroatoms independently selected from O, N, and S, optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹. In some embodiments, o=2; p=2 or 3; and two pairs, each of one R⁶′ and one R⁷′, are on adjacent atoms, and each pair of one R⁶′ and one R⁷′ taken together with the atoms connecting them form a 5-membered aliphatic heterocyclic ring containing 1 or 2 heteroatoms independently selected from O, N, and S. In some embodiments, o=2; p=2 or 3; and two pairs, each of one R⁶′ and one R⁷′, are on adjacent atoms, and each pair of one R⁶′ and one R⁷′ taken together with the atoms connecting them form a 5-membered heteroaromatic ring containing 1 or 2 heteroatoms independently selected from O, N, and S. In some embodiments, o=2; p=2 or 3; and two pairs, each of one R⁶′ and one R⁷′, are on adjacent atoms, and each pair of one R⁶′ and one R⁷′ taken together with the atoms connecting them form a 6-membered heterocyclic ring containing 1 or 2 heteroatoms independently selected from O, N, and S, optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹. In some embodiments, o=2; p=2 or 3; and two pairs, each of one R⁶′ and one R⁷′, are on adjacent atoms, and each pair of one R⁶′ and one R⁷′ taken together with the atoms connecting them form a 6-membered aliphatic heterocyclic ring containing 1 or 2 heteroatoms independently selected from O, N, and S. In some embodiments, o=2; p=2 or 3; and two pairs, each of one R⁶′ and one R⁷′, are on adjacent atoms, and each pair of one R⁶′ and one R⁷′ taken together with the atoms connecting them form a 6-membered heteroaromatic ring containing 1 or 2 heteroatoms independently selected from O, N, and S. In some embodiments, o=2; p=2 or 3; and two pairs, each of one R⁶′ and one R⁷′, are on adjacent atoms, and each pair of one R⁶′ and one R⁷′ taken together with the atoms connecting them independently form a C₄-C₈ carbocyclic ring or a 5- to 8-membered heterocyclic ring containing 1 or 2 heteroatoms independently selected from O, N, and S, wherein one of the two rings is fused to the B ring at the 2- and 3-positions relative to the bond connecting the B ring to the CR⁴R⁵ group, and the other of the two rings is fused to the B ring at the 5- and 6-positions relative to the bond connecting the B ring to the CR⁴R⁵ group. In some embodiments, o=2; p=2; and two pairs, each of one R⁶′ and one R⁷′, are on adjacent atoms, and each pair of one R⁶′ and one R⁷′ taken together with the atoms connecting them form a C₅ aliphatic carbocyclic ring. In some embodiments, o=2; p=3; and two pairs, each of one R⁶′ and one R⁷′, are on adjacent atoms, and each pair of one R⁶′ and one R⁷′ taken together with the atoms connecting them form a C₅ aliphatic carbocyclic ring; and one R⁷′ is Cl, Br, or I, (e.g., Cl). In some embodiments, one R⁷′ is pyrazolyl and is para to the bond connecting the B ring to the CR⁴R⁵ group of Formula AA. In some embodiments, one R⁷′ is 3-pyrazolyl and is para to the bond connecting the B ring to the CR⁴R⁵ group of Formula AA. In some embodiments, one R⁷′ is 4-pyrazolyl and is para to the bond connecting the B ring to the CR⁴R⁵ group of Formula AA. In some embodiments, one R⁷′ is 5-pyrazolyl and is para to the bond connecting the B ring to the CR⁴R⁵ group of Formula AA. In some embodiments, one R⁷′ is thiazolyl and is para to the bond connecting the B ring to the CR⁴R⁵ group of Formula AA. In some embodiments, one R⁷′ is 4-thiazolyl and is para to the bond connecting the B ring to the CR⁴R⁵ group of Formula AA. In some embodiments, one R⁷′ is 5-thiazolyl and is para to the bond connecting the B ring to the CR⁴R⁵ group of Formula AA. In some embodiments, one R⁷′ is furyl and is para to the bond connecting the B ring to the CR⁴R⁵ group of Formula AA. In some embodiments, one R⁷′ is 2-furyl and is para to the bond connecting the B ring to the CR⁴R⁵ group of Formula AA. In some embodiments, one R⁷′ is thiophenyl and is para to the bond connecting the B ring to the CR⁴R⁵ group of Formula AA. In some embodiments, one R⁷′ is 2-thiophenyl and is para to the bond connecting the B ring to the CR⁴R⁵ group of Formula AA. In some embodiments, one R⁷′ is phenyl and is para to the bond connecting the B ring to the CR⁴R⁵ group of Formula AA. In some embodiments, one R⁷′ is cycloalkenyl (e.g., cyclopentenyl, e.g., 1-cyclopentenyl) and is para to the bond connecting the B ring to the CR⁴R⁵ group of Formula AA. In some embodiments, one R⁷′ is phenyl optionally substituted with one or more C₁-C₆ alkyl (e.g., methyl or propyl, e.g., 2-propyl) optionally substituted with one or more hydroxyl, NR⁸R⁹ (e.g., dimethylamino), or C₆-C₁₀ aryl (e.g., phenyl, naphthyl, or methylenedioxyphenyl) and is para to the bond connecting the B ring to the CR⁴R⁵ group of Formula AA. In some embodiments, one R⁷′ is phenyl optionally substituted with one or more C₁-C₆ alkoxy (e.g., methoxy) optionally substituted with one or more hydroxyl, NR⁸R⁹ (e.g., dimethylamino), or C₆-C₁₀ aryl (e.g., phenyl, naphthyl, or methylenedioxyphenyl) and is para to the bond connecting the B ring to the CR⁴R⁵ group of Formula AA. In some embodiments, one R⁷′ is phenyl optionally substituted with one or more C₆-C₁₀ aryloxy (e.g., phenoxy) and is para to the bond connecting the B ring to the CR⁴R⁵ group of Formula AA. In some embodiments, one R⁷′ is phenyl optionally substituted with one or more CN and is para to the bond connecting the B ring to the CR⁴R⁵ group of Formula AA. In some embodiments, one R⁷′ is phenyl optionally substituted with one or more halo (e.g., F, Cl) and is para to the bond connecting the B ring to the CR⁴R⁵ group of Formula AA and is para to the bond connecting the B ring to the CR⁴R⁵ group of Formula AA. In some embodiments, one R⁷′ is phenyl optionally substituted with one or more COOC₁-C₆ alkyl (e.g., CO₂t-Bu) and is para to the bond connecting the B ring to the CR⁴R⁵ group of Formula AA. In some embodiments, one R⁷′ is phenyl optionally substituted with one or more S(O₂)C₁-C₆ alkyl (e.g., S(O₂)methyl) and is para to the bond connecting the B ring to the CR⁴R⁵ group of Formula AA. In some embodiments, one R⁷′ is phenyl optionally substituted with one or more 3- to 7-membered heterocycloalkyl (e.g., morpholinyl) and is para to the bond connecting the B ring to the CR⁴R⁵ group of Formula AA. In some embodiments, one R⁷′ is phenyl optionally substituted with one or more CONR⁸R⁹ (e.g., unsubstituted amido) and is para to the bond connecting the B ring to the CR⁴R⁵ group of Formula AA. In some embodiments, one R⁷′ is phenyl optionally substituted with one or more C₁-C₆ alkyl (e.g., methyl or propyl, e.g., 2-propyl) and with one or more halo (e.g., F, Cl) and is para to the bond connecting the B ring to the CR⁴R⁵ group of Formula AA and is para to the bond connecting the B ring to the CR⁴R⁵ group of Formula AA.

In some embodiments, R⁶′ and R⁷′ are each attached to a carbon of an aryl ring B. In some embodiments, R⁶′ and R⁷′ are each attached to a carbon of a heteroaryl ring B. In some embodiments, R⁶′ is attached to a carbon and R⁷′ is attached to a nitrogen of a heteroaryl ring B. In some embodiments, R⁷′ is attached to a carbon and R⁶′ is attached to a nitrogen of a heteroaryl ring B.

In some embodiments, the optionally substituted ring B is

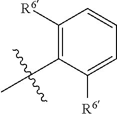

and each R^{6′} is independently selected from the group consisting of: C₁-C₆ alkyl, C₁-C₆ haloalkyl, C₁-C₆ alkoxy, C₁-C₆ haloalkoxy, Cl, Br, I, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₁₀ cycloalkyl and 3- to 10-membered heterocycloalkyl, and C₂-C₆ alkenyl,
wherein R^{6′} is optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, O(C₃-C₁₀ cycloalkyl), and S(O₂)C₁-C₆ alkyl; and wherein the C₁-C₆ alkyl or C₁-C₆ alkoxy that R⁶ is substituted with is optionally substituted with one or more hydroxyl, halo, C₆-C₁₀ aryl or NR⁸R⁹, or wherein R⁶ is optionally fused to a five-to-seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;

In some embodiments, the optionally substituted ring B is

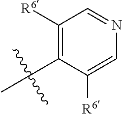

and each R⁶′ is independently selected from the group consisting of: C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl, wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆, alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl.

In some embodiments, the optionally substituted ring B

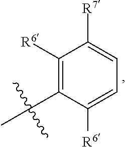

wherein each R^{6′} and R^{7′} are independently selected from C₁-C₆ alkyl, C₁-C₆ haloalkyl, C₁-C₆ alkoxy, C₁-C₆ haloalkoxy, Cl, Br, I, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₁₀ cycloalkyl and 3- to 10-membered heterocycloalkyl, and C₂-C₆ alkenyl,
wherein R^{6′} and R^{7′} are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, O(C₃-C₁₀ cycloalkyl), and S(O₂)C₁-C₆ alkyl; and wherein the C₁-C₆ alkyl or C₁-C₆ alkoxy that R^{6′} or R^{7′} is substituted with is optionally substituted with one or more hydroxyl, halo, C₆-C₁₀ aryl or NR⁸R⁹, or wherein R^{6′} or R^{7′} is optionally fused to a five-to-seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or one pair of R^{6′} and R^{7′} on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments, the optionally substituted ring B is

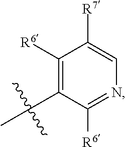

wherein each R^{6′} and R^{7′} are independently selected from C₁-C₆ alkyl, C₁-C₆ haloalkyl, C₁-C₆ alkoxy, C₁-C₆ haloalkoxy, Cl, Br, I, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₁₀ cycloalkyl and 3- to 10-membered heterocycloalkyl, and C₂-C₆ alkenyl,
wherein R^{6′} and R^{7′} are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, O(C₃-C₁₀ cycloalkyl), and S(O₂)C₁-C₆ alkyl; and wherein the C₁-C₆ alkyl or C₁-C₆ alkoxy that R^{6′} or R^{7′} is substituted with is optionally substituted with one or more hydroxyl, halo, C₆-C₁₀ aryl or NR⁸R⁹, or wherein R^{6′} or R^{7′} is optionally fused to a five-to-seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or one pair of R^{6′} and R^{7′} on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments, the optionally substituted ring B is

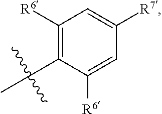

wherein each R^{6′} and R^{7′} are independently selected from C₁-C₆ alkyl, C₁-C₆ haloalkyl, C₁-C₆ alkoxy, C₁-C₆ haloalkoxy, Cl, Br, I, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₁₀ cycloalkyl and 3- to 10-membered heterocycloalkyl, and C₂-C₆ alkenyl,
wherein R^{6′} and R^{7′} are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, O(C₃-C₁₀ cycloalkyl), and S(O₂)C₁-C₆ alkyl; and wherein the C₁-C₆ alkyl or C₁-C₆ alkoxy that R^{6′} or R^{7′} is substituted with is optionally substituted with one or more hydroxyl, halo, C₆-C₁₀ aryl or NR⁸R⁹, or wherein R^{6′} or R^{7′} is optionally fused to a five-to-seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl.

In some embodiments, the optionally substituted ring B is

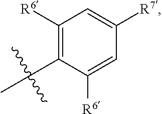

wherein each R⁶′ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;

wherein R^{7′} is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy, and wherein R⁷ is optionally fused to a five-to-seven-membered carbocyclic ring or heterocyclic ring containing one or two heteroatoms independently selected from oxygen, sulfur and nitrogen.

In some embodiments, the optionally substituted ring B is

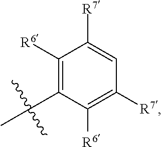

wherein each R^{6′} is independently selected from R^{6′} and R^{7′} are independently selected from C₁-C₆ alkyl, C₁-C₆, haloalkyl, C₁-C₆ alkoxy, C₁-C₆ haloalkoxy, Cl, Br, I, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₁₀ cycloalkyl and 3- to 10-membered heterocycloalkyl, and C₂-C₆ alkenyl,
wherein R^{6′} and R^{7′} are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, O(C₃-C₁₀ cycloalkyl), and S(O₂)C₁-C₆ alkyl; and wherein the C₁-C₆ alkyl or C₁-C₆ alkoxy that R^{6′} or R^{7′} is substituted with is optionally substituted with one or more hydroxyl, halo, C₆-C₁₀ aryl or NR⁸R⁹, or wherein R^{6′} or R^{7′} is optionally fused to a five-to-seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
or at least one pair of R^{6′} and R^{7′} on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments, the optionally substituted ring B is

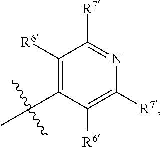

wherein each R^{6′} and R^{7′} are independently selected from C₁-C₆ alkyl, C₁-C₆ haloalkyl, C₁-C₆ alkoxy, C₁-C₆ haloalkoxy, Cl, Br, I, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₁₀ cycloalkyl and 3- to 10-membered heterocycloalkyl, and C₂-C₆ alkenyl,
wherein R^{6′} and R^{7′} are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, O(C₃-C₁₀ cycloalkyl), and S(O₂)C₁-C₆ alkyl; and wherein the C₁-C₆ alkyl or C₁-C₆ alkoxy that R^{6′} or R^{7′} is substituted with is optionally substituted with one or more hydroxyl, halo, C₆-C₁₀ aryl or NR⁸R⁹, or wherein R^{6′} or R^{7′} is optionally fused to a five-to-seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
or at least one pair of R^{6′} and R^{7′} on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments, the optionally substituted ring B is

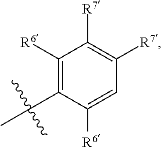

wherein each R^{6′} and R^{7′} are independently selected from C₁-C₆ alkyl, C₁-C₆ haloalkyl, C₁-C₆ alkoxy, C₁-C₆ haloalkoxy, Cl, Br, I, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₁₀ cycloalkyl and 3- to 10-membered heterocycloalkyl, and C₂-C₆ alkenyl,
wherein R^{6′} and R^{7′} are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, O(C₃-C₁₀ cycloalkyl), and S(O₂)C₁-C₆ alkyl; and wherein the C₁-C₆ alkyl or C₁-C₆ alkoxy that R^{6′} or R^{7′} is substituted with is optionally substituted with one or more hydroxyl, halo, C₆-C₁₀ aryl or NR⁸R⁹, or wherein R^{6′} or R^{7′} is optionally fused to a five-to-seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or at least one pair of R^{6′} and R^{7′} on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments, the optionally substituted ring B is

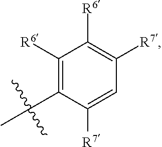

wherein each R^{6′} and R^{7′} are independently selected from C₁-C₆ alkyl, C₁-C₆ haloalkyl, C₁-C₆ alkoxy, C₁-C₆ haloalkoxy, Cl, Br, I, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₁₀ cycloalkyl and 3- to 10-membered heterocycloalkyl, and C₂-C₆ alkenyl,
wherein R^{6′} and R^{7′} are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, O(C₃-C₁₀ cycloalkyl), and S(O₂)C₁-C₆ alkyl; and wherein the C₁-C₆ alkyl or C₁-C₆ alkoxy that R^{6′} or R^{7′} is substituted with is optionally substituted with one or more hydroxyl, halo, C₆-C₁₀ aryl or NR⁸R⁹, or wherein R^{6′} or R^{7′} is optionally fused to a five-to-seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or one pair of R^{6′} and R^{7′} on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments, the optionally substituted ring B is

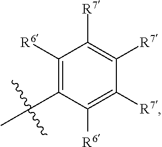

wherein each R^{6′} R^{6′} and R^{7′} are independently selected from C₁-C₆ alkyl, C₁-C₆ haloalkyl, C₁-C₆ alkoxy, C₁-C₆ haloalkoxy, Cl, Br, I, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₁₀ cycloalkyl and 3- to 10-membered heterocycloalkyl, and C₂-C₆ alkenyl,
wherein R^{6′} and R^{7′} are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, O(C₃-C₁₀ cycloalkyl), and S(O₂)C₁-C₆ alkyl; and wherein the C₁-C₆ alkyl or C₁-C₆ alkoxy that R^{6′} or R^{7′} is substituted with is optionally substituted with one or more hydroxyl, halo, C₆-C₁₀ aryl or NR⁸R⁹, or wherein R^{6′} or R^{7′} is optionally fused to a five-to-seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
or at least one pair of R^{6′} and R^{7′} on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.
   The Groups R⁴ and R⁵

In some embodiments, each of R⁴ and R⁵ is independently selected from hydrogen and C₁-C₆ alkyl; or R⁴ and R⁵, together with the carbon atom to which they are attached, form a C₃-C₈ cycloalkyl optionally independently substituted with one or more substituents independently selected from hydroxy, hydroxymethyl, halo, oxo, C₁-C₆ alkyl, C₁-C₆ alkoxy, and NR⁸R⁹. In some embodiments, each of R⁴ and R⁵ is independently selected from hydrogen and C₁-C₆, alkyl.

In some embodiments, R⁴ is hydrogen. In some embodiments, R⁵ is hydrogen. In some embodiments, each of R⁴ and R⁵ is hydrogen. In some embodiments, R⁴ is C₁-C₆ alkyl. In some embodiments, R⁵ is C₁-C₆ alkyl. In some embodiments, each of R⁴ and R⁵ is C₁-C₆ alkyl, In some embodiments, R⁴ is hydrogen and R⁵ is C₁-C₆ alkyl. In some embodiments, R⁴ is hydrogen and R⁵ is C₁-C₆ alkyl, and the carbon bonded to R⁴ and R⁵ has (S) stereochemistry. In some embodiments, R⁴ is hydrogen and R⁵ is C₁-C₆ alkyl, and the carbon bonded to R⁴ and R⁵ has (R) stereochemistry. In some embodiments, R⁴ and R⁵, together with the carbon atom to which they are attached, form a C₃-C₈ cycloalkyl optionally independently substituted with one or more substituents independently selected from hydroxy, hydroxymethyl, halo, oxo, C₁-C₆ alkyl, C₁-C₆ alkoxy, and NR⁸R⁹

### The Group R¹⁰

In some embodiments, R¹⁰ is C₁-C₆ alkyl. In some embodiments, R¹⁰ is methyl. In some embodiments, R¹⁰ is ethyl.

The Groups R⁸ and R⁹

In some embodiments, each of R⁸ and R⁹ at each occurrence is independently selected from hydrogen, C₁-C₆ alkyl, (C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R⁸ and R⁹ taken together with the nitrogen they are attached to form a 3- to 7-membered ring optionally containing one or more heteroatoms in addition to the nitrogen they are attached to. In some embodiments, each of R⁸ and R⁹ at each occurrence is hydrogen, In some embodiments, each R⁸ at each occurrence is hydrogen and each R⁹ at each occurrence is C₁-C₆ alkyl. In some embodiments, each R⁸ at each occurrence is hydrogen and each R⁹ at each occurrence is methyl. In some embodiments, each R⁸ at each occurrence is hydrogen and each R⁹ at each occurrence is ethyl. In some embodiments, each of R⁸ and R⁹ at each occurrence is methyl. In some embodiments, each of R⁸ and R⁹ at each occurrence is ethyl. In some embodiments, R⁸ and R⁹ taken together with the nitrogen they are attached to form a 3-membered ring. In some embodiments, R⁸ and R⁹ taken together with the nitrogen they are attached to form a 4-membered ring. In some embodiments, R⁸ and R⁹ taken together with the nitrogen they are attached to form a 5-membered ring. In some embodiments, R⁸ and R⁹ taken together with the nitrogen they are attached to form a 6-membered ring optionally containing one or more oxygen atoms in addition to the nitrogen they are attached to. In some embodiments, R⁸ and R⁹ taken together with the nitrogen they are attached to form a 6-membered ring optionally containing one or more nitrogen atoms in addition to the nitrogen they are attached to. In some embodiments, R⁸ and R⁹ taken together with the nitrogen they are attached to form a 7-membered ring.

### The Group R¹³

In some embodiments, R¹³ is C₁-C₆ alkyl. In some embodiments, R¹³ is methyl. In some embodiments, R¹³ is ethyl. In some embodiments, R¹³ is C₆-C₁₀ aryl. In some embodiments, R¹³ is phenyl. In some embodiments, R¹³ is 5- to 10-membered heteroaryl.

The Groups R¹¹ and R¹²

In some embodiments, each of R¹¹ and R¹² at each occurrence is independently selected from hydrogen and C₁-C₆ alkyl optionally substituted with hydroxy, OR¹³, or O—(C₁-C₆ alkyl)-R¹³. In some embodiments, each of R¹¹ and R¹² at each occurrence is independently selected from hydrogen and C₁-C₆ alkyl optionally substituted with hydroxy. In some embodiments, each of R¹¹ and R¹² at each occurrence is independently selected from hydrogen and unsubstituted C₁-C₆, alkyl In some embodiments, each of R¹¹ and R¹² at each occurrence is hydrogen, In some embodiments, each R¹¹ at each occurrence is hydrogen and each R¹² at each occurrence is C₁-C₆ alkyl. In some embodiments, each R¹¹ at each occurrence is hydrogen and each R¹² at each occurrence is methyl. In some embodiments, each R¹¹ at each occurrence is hydrogen and each R¹² at each occurrence is ethyl. In some embodiments, each R¹¹ at each occurrence is hydrogen and each R¹² at each occurrence is hydroxyethyl. In some embodiments, each of R¹¹ and R¹² at each occurrence is methyl. In some embodiments, each of R¹¹ and R¹² at each occurrence is ethyl.

In some embodiments of the compound of formula AA,

the substituted ring A is

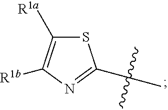

and R^{1a} and R^{1b} are one of the following combinations:
R^{1a} is hydroxymethyl, and R^{1b} is hydroxymethyl; R^{1a} is hydroxymethyl, and R^{1b} is hydroxyethyl; R^{1a} is hydroxymethyl, and R^{1b} is 2-hydroxy-2-propyl; R^{1a} is hydroxymethyl, and R^{1b} is 3-hydroxy-2-propyl; R^{1a} is hydroxymethyl, and R^{1b} is 1-hydroxy-1-propyl; R^{1a} is hydroxymethyl, and R^{1b} is 2-hydroxy-1-propyl; R^{1a} is hydroxymethyl, and R^{1b} is 3-hydroxy-1-propyl; R^{1a} is hydroxymethyl, and R^{1b} is hydroxybutyl; R^{1a} is hydroxymethyl, and R^{1b} is hydroxypentyl; R^{1a} is hydroxymethyl, and R^{1b} is hydroxyhexyl; R^{1a} is hydroxyethyl, and R^{1b} is hydroxymethyl; R^{1a} is hydroxyethyl, and R^{1b} is hydroxyethyl; R^{1a} is hydroxyethyl, and R^{1b} is 2-hydroxy-2-propyl; R^{1a} is hydroxyethyl, and R^{1b} is 3-hydroxy-2-propyl; R^{1a} is hydroxyethyl, and R^{1b} is 1-hydroxy-1-propyl; R^{1a} is hydroxyethyl, and R^{1b} is 2-hydroxy-1-propyl; R^{1a} is hydroxyethyl, and R^{1b} is 3-hydroxy-1-propyl; R^{1a} is hydroxyethyl, and R^{1b} is hydroxybutyl; R^{1a} is hydroxyethyl, and R^{1b} is hydroxypentyl; R^{1a} is hydroxyethyl, and R^{1b} is hydroxyhexyl; R^{1a} is 2-hydroxy-2-propyl, and R^{1b} is hydroxymethyl; R^{1a} is 2-hydroxy-2-propyl, and R^{1b} is hydroxyethyl; R^{1a} is 2-hydroxy-2-propyl, and R^{1b} is 2-hydroxy-2-propyl; R^{1a} is 2-hydroxy-2-propyl, and R^{1b} is 3-hydroxy-2-propyl; R^{1a} is 2-hydroxy-2-propyl, and R^{1b} is 1-hydroxy-1-propyl; R^{1a} is 2-hydroxy-2-propyl, and R^{1b} is 2-hydroxy-1-propyl; R^{1a} is 2-hydroxy-2-propyl, and R^{1b} is 3-hydroxy-1-propyl; R^{1a} is 2-hydroxy-2-propyl, and R^{1b} is hydroxybutyl; R^{1a} is 2-hydroxy-2-propyl, and R^{1b} is hydroxypentyl; R^{1a} is 2-hydroxy-2-propyl, and R^{1b} is hydroxyhexyl; R^{1b} is hydroxymethyl, and R^{1a} is hydroxymethyl; R^{1b} is hydroxymethyl, and R^{1a} is hydroxy ethyl; R^{1b} is hydroxymethyl, and R^{1a} is 2-hydroxy-2-propyl; R^{1b} is hydroxymethyl, and R^{1a} is 3-hydroxy-2-propyl; R^{1b} is hydroxymethyl, and R^{1a} is 1-hydroxy-1-propyl; R^{1b} is hydroxymethyl, and R^{1a} is 2-hydroxy-1-propyl; R^{1b} is hydroxymethyl, and R^{1a} is 3-hydroxy-1-propyl; R^{1b} is hydroxymethyl, and R^{1a} is hydroxybutyl; R^{1b} is hydroxymethyl, and R^{1a} is hydroxypentyl; R^{1b} is hydroxymethyl, and R^{1a} is hydroxyhexyl; R^{1b} is hydroxy ethyl, and R^{1a} is hydroxymethyl; R^{1b} is hydroxy ethyl, and R^{1a} is hydroxyethyl; R^{1b} is hydroxyethyl, and R^{1a} is 2-hydroxy-2-propyl; R^{1b} is hydroxyethyl, and R^{1a} is 3-hydroxy-2-propyl; R^{1b} is hydroxyethyl, and R^{1a} is 1-hydroxy-1-propyl; R^{1b} is hydroxyethyl, and R^{1a} is 2-hydroxy-1-propyl; R^{1b} is hydroxyethyl, and R^{1a} is 3-hydroxy-1-propyl; R^{1b} is hydroxyethyl, and R^{1a} is hydroxybutyl; R^{1b} is hydroxyethyl, and R^{1a} is hydroxypentyl; R^{1b} is hydroxyethyl, and R^{1a} is hydroxyhexyl; R^{1b} is 2-hydroxy-2-propyl, and R^{1a} is hydroxymethyl; R^{1b} is 2-hydroxy-2-propyl, and R^{1a} is hydroxyethyl; R^{1b} is 2-hydroxy-2-propyl, and R^{1a} is 2-hydroxy-2-propyl; R^{1b} is 2-hydroxy-2-propyl, and R^{1a} is 3-hydroxy-2-propyl; R^{1b} is 2-hydroxy-2-propyl, and R^{1a} is 1-hydroxy-1-propyl; R^{1b} is 2-hydroxy-2-propyl, and R^{1a} is 2-hydroxy-1-propyl; R^{1b} is 2-hydroxy-2-propyl, and R^{1a} is 3-hydroxy-1-propyl; R^{1b} is 2-hydroxy-2-propyl, and R^{1a} is hydroxybutyl; R^{1b} is 2-hydroxy-2-propyl, and R^{1a} is hydroxypentyl; and R^{1b} is 2-hydroxy-2-propyl, and R^{1a} is hydroxyhexyl.

In some embodiments of the compound of formula AA,

the substituted ring A is

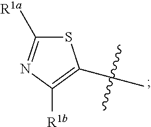

and R^{1a} and R^{1b} are one of the following combinations:
R^{1a} is hydroxymethyl, and R^{1b} is hydroxymethyl; R^{1a} is hydroxymethyl, and R^{1b} is hydroxyethyl; R^{1a} is hydroxymethyl, and R^{1b} is 2-hydroxy-2-propyl; R^{1a} is hydroxymethyl, and R^{1b} is 3-hydroxy-2-propyl; R^{1a} is hydroxymethyl, and R^{1b} is 1-hydroxy-1-propyl; R^{1a} is hydroxymethyl, and R^{1b} is 2-hydroxy-1-propyl; R^{1a} is hydroxymethyl, and R^{1b} is 3-hydroxy-1-propyl; R^{1a} is hydroxymethyl, and R^{1b} is hydroxybutyl; R^{1a} is hydroxymethyl, and R^{1b} is hydroxypentyl; R^{1a} is hydroxymethyl, and R^{1b} is hydroxyhexyl; R^{1a} is hydroxyethyl, and R^{1b} is hydroxymethyl; R^{1a} is hydroxy ethyl, and R^{1b} is hydroxy ethyl; R^{1a} is hydroxy ethyl, and R^{1b} is 2-hydroxy-2-propyl; R^{1a} is hydroxy ethyl, and R^{1b} is 3-hydroxy-2-propyl; R^{1a} is hydroxyethyl, and R^{1b} is 1-hydroxy-1-propyl; R^{1a} is hydroxyethyl, and R^{1b} is 2-hydroxy-1-propyl; R^{1a} is hydroxyethyl, and R^{1b} is 3-hydroxy-1-propyl; R^{1a} is hydroxyethyl, and R^{1b} is hydroxybutyl; R^{1a} is hydroxyethyl, and R^{1b} is hydroxypentyl; R^{1a} is hydroxyethyl, and R^{1b} is hydroxyhexyl; R^{1a} is 2-hydroxy-2-propyl, and R^{1b} is hydroxymethyl; R^{1a} is 2-hydroxy-2-propyl, and R^{1b} is hydroxyethyl; R^{1a} is 2-hydroxy-2-propyl, and R^{1b} is 2-hydroxy-2-propyl; R^{1a} is 2-hydroxy-2-propyl, and R^{1b} is 3-hydroxy-2-propyl; R^{1a} is 2-hydroxy-2-propyl, and R^{1b} is 1-hydroxy-1-propyl; R^{1a} is 2-hydroxy-2-propyl, and R^{1b} is 2-hydroxy-1-propyl; R^{1a} is 2-hydroxy-2-propyl, and R^{1b} is 3-hydroxy-1-propyl; R^{1a} is 2-hydroxy-2-propyl, and R^{1b} is hydroxybutyl; R^{1a} is 2-hydroxy-2-propyl, and R^{1b} is hydroxypentyl; R^{1a} is 2-hydroxy-2-propyl, and R^{1b} is hydroxyhexyl; R^{1b} is hydroxymethyl, and R^{1a} is hydroxymethyl; R^{1b} is hydroxymethyl, and R^{1a} is hydroxyethyl; R^{1b} is hydroxymethyl, and R^{1a} is 2-hydroxy-2-propyl; R^{1b} is hydroxymethyl, and R^{1a} is 3-hydroxy-2-propyl; R^{1b} is hydroxymethyl, and R^{1a} is 1-hydroxy-1-propyl; R^{1b} is hydroxymethyl, and R^{1a} is 2-hydroxy-1-propyl; R^{1b} is hydroxymethyl, and R^{1a} is 3-hydroxy-1-propyl; R^{1b} is hydroxymethyl, and R^{1a} is hydroxybutyl; R^{1b} is hydroxymethyl, and R^{1a} is hydroxypentyl; R^{1b} is hydroxymethyl, and R^{1a} is hydroxyhexyl; R^{1b} is hydroxyethyl, and R^{1a} is hydroxymethyl; R^{1b} is hydroxyethyl, and R^{1a} is hydroxyethyl; R^{1b} is hydroxyethyl, and R^{1a} is 2-hydroxy-2-propyl; R^{1b} is hydroxyethyl, and R^{1a} is 3-hydroxy-2-propyl; R^{1b} is hydroxyethyl, and R^{1a} is 1-hydroxy-1-propyl; R^{1b} is hydroxyethyl, and R^{1a} is 2-hydroxy-1-propyl; R^{1b} is hydroxyethyl, and R^{1a} is 3-hydroxy-1-propyl; R^{1b} is hydroxyethyl, and R^{1a} is hydroxybutyl; R^{1b} is hydroxyethyl, and R^{1a} is hydroxypentyl; R^{1b} is hydroxyethyl, and R^{1a} is hydroxyhexyl; R^{1b} is 2-hydroxy-2-propyl, and R^{1a} is hydroxymethyl; R^{1b} is 2-hydroxy-2-propyl, and R^{1a} is hydroxyethyl; R^{1b} is 2-hydroxy-2-propyl, and R^{1a} is 2-hydroxy-2-propyl; R^{1b} is 2-hydroxy-2-propyl, and R^{1a} is 3-hydroxy-2-propyl; R^{1b} is 2-hydroxy-2-propyl, and R^{1a} is 1-hydroxy-1-propyl; R^{1b} is 2-hydroxy-2-propyl, and R^{1a} is 2-hydroxy-1-propyl; R^{1b} is 2-hydroxy-2-propyl, and R^{1a} is 3-hydroxy-1-propyl; R^{1b} is 2-hydroxy-2-propyl, and R^{1a} is hydroxybutyl; R^{1b} is 2-hydroxy-2-propyl, and R^{1a} is hydroxypentyl; and R^{1b} is 2-hydroxy-2-propyl, and R^{1a} is hydroxyhexyl.

In some embodiments of the compound of formula AA,

the substituted ring A is

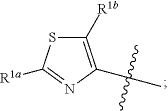

and R^{1a} and R^{1b} are one of the following combinations:
R^{1a} is hydroxymethyl, and R^{1b} is hydroxymethyl; R^{1a} is hydroxymethyl, and R^{1b} is hydroxy ethyl; R^{1a} is hydroxymethyl, and R^{1b} is 2-hydroxy-2-propyl; R^{1a} is hydroxymethyl, and R^{1b} is 3-hydroxy-2-propyl; R^{1a} is hydroxymethyl, and R^{1b} is 1-hydroxy-1-propyl; R^{1a} is hydroxymethyl, and R^{1b} is 2-hydroxy-1-propyl; R^{1a} is hydroxymethyl, and R^{1b} is 3-hydroxy-1-propyl; R^{1a} is hydroxymethyl, and R^{1b} is hydroxybutyl; R^{1a} is hydroxymethyl, and R^{1b} is hydroxypentyl; R^{1a} is hydroxymethyl, and R^{1b} is hydroxyhexyl; R^{1a} is hydroxy ethyl, and R^{1b} is hydroxymethyl; R^{1a} is hydroxy ethyl, and R^{1b} is hydroxy ethyl; R^{1a} is hydroxy ethyl, and R^{1b} is 2-hydroxy-2-propyl; R^{1a} is hydroxy ethyl, and R^{1b} is 3-hydroxy-2-propyl; R^{1a} is hydroxyethyl, and R^{1b} is 1-hydroxy-1-propyl; R^{1a} is hydroxyethyl, and R^{1b} is 2-hydroxy-1-propyl; R^{1a} is hydroxyethyl, and R^{1b} is 3-hydroxy-1-propyl; R^{1a} is hydroxyethyl, and R^{1b} is hydroxybutyl; R^{1a} is hydroxyethyl, and R^{1b} is hydroxypentyl; R^{1a} is hydroxyethyl, and R^{1b} is hydroxyhexyl; R^{1a} is 2-hydroxy-2-propyl, and R^{1b} is hydroxymethyl; R^{1a} is 2-hydroxy-2-propyl, and R^{1b} is hydroxyethyl; R^{1a} is 2-hydroxy-2-propyl, and R^{1b} is 2-hydroxy-2-propyl; R^{1a} is 2-hydroxy-2-propyl, and R^{1b} is 3-hydroxy-2-propyl; R^{1a} is 2-hydroxy-2-propyl, and R^{1b} is 1-hydroxy-1-propyl; R^{1a} is 2-hydroxy-2-propyl, and R^{1b} is 2-hydroxy-1-propyl; R^{1a} is 2-hydroxy-2-propyl, and R^{1b} is 3-hydroxy-1-propyl; R^{1a} is 2-hydroxy-2-propyl, and R^{1b} is hydroxybutyl; R^{1a} is 2-hydroxy-2-propyl, and R^{1b} is hydroxypentyl; R^{1a} is 2-hydroxy-2-propyl, and R^{1b} is hydroxyhexyl; R^{1b} is hydroxymethyl, and R^{1a} is hydroxymethyl; R^{1b} is hydroxymethyl, and R^{1a} is hydroxyethyl; R^{1b} is hydroxymethyl, and R^{1a} is 2-hydroxy-2-propyl; R^{1b} is hydroxymethyl, and R^{1a} is 3-hydroxy-2-propyl; R^{1b} is hydroxymethyl, and R^{1a} is 1-hydroxy-1-propyl; R^{1b} is hydroxymethyl, and R^{1a} is 2-hydroxy-1-propyl; R^{1b} is hydroxymethyl, and R^{1a} is 3-hydroxy-1-propyl; R^{1b} is hydroxymethyl, and R^{1a} is hydroxybutyl; R^{1b} is hydroxymethyl, and R^{1a} is hydroxypentyl; R^{1b} is hydroxymethyl, and R^{1a} is hydroxyhexyl; R^{1b} is hydroxyethyl, and R^{1a} is hydroxymethyl; R^{1b} is hydroxyethyl, and R^{1a} is hydroxyethyl; R^{1b} is hydroxyethyl, and R^{1a} is 2-hydroxy-2-propyl; R^{1b} is hydroxyethyl, and R^{1a} is 3-hydroxy-2-propyl; R^{1b} is hydroxyethyl, and R^{1a} is 1-hydroxy-1-propyl; R^{1b} is hydroxyethyl, and R^{1a} is 2-hydroxy-1-propyl; R^{1b} is hydroxyethyl, and R^{1a} is 3-hydroxy-1-propyl; R^{1b} is hydroxy ethyl, and R^{1a} is hydroxybutyl; R^{1b} is hydroxy ethyl, and R^{1a} is hydroxypentyl; R^{1b} is hydroxyethyl, and R^{1a} is hydroxyhexyl; R^{1b} is 2-hydroxy-2-propyl, and R^{1a} is hydroxymethyl; R^{1b} is 2-hydroxy-2-propyl, and R^{1a} is hydroxyethyl; R^{1b} is 2-hydroxy-2-propyl, and R^{1a} is 2-hydroxy-2-propyl; R^{1b} is 2-hydroxy-2-propyl, and R^{1a} is 3-hydroxy-2-propyl; R^{1b} is 2-hydroxy-2-propyl, and R^{1a} is 1-hydroxy-1-propyl; R^{1b} is 2-hydroxy-2-propyl, and R^{1a} is 2-hydroxy-1-propyl; R^{1b} is 2-hydroxy-2-propyl, and R^{1a} is 3-hydroxy-1-propyl; R^{1b} is 2-hydroxy-2-propyl, and R^{1a} is hydroxybutyl; R^{1b} is 2-hydroxy-2-propyl, and R^{1a} is hydroxypentyl; and R^{1b} is 2-hydroxy-2-propyl, and R^{1a} is hydroxyhexyl.

In some embodiments of the compound of formula AA,

the substituted ring A is

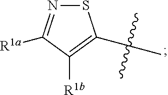

and R^{1a} and R^{1b} are one of the following combinations:
R^{1a} is hydroxymethyl, and R^{1b} is hydroxymethyl; R^{1a} is hydroxymethyl, and R^{1b} is hydroxyethyl; R^{1a} is hydroxymethyl, and R^{1b} is 2-hydroxy-2-propyl; R^{1a} is hydroxymethyl, and R^{1b} is 3-hydroxy-2-propyl; R^{1a} is hydroxymethyl, and R^{1b} is 1-hydroxy-1-propyl; R^{1a} is hydroxymethyl, and R^{1b} is 2-hydroxy-1-propyl; R^{1a} is hydroxymethyl, and R^{1b} is 3-hydroxy-1-propyl; R^{1a} is hydroxymethyl, and R^{1b} is hydroxybutyl; R^{1a} is hydroxymethyl, and R^{1b} is hydroxypentyl; R^{1a} is hydroxymethyl, and R^{1b} is hydroxyhexyl; R^{1a} is hydroxyethyl, and R^{1b} is hydroxymethyl; R^{1a} is hydroxyethyl, and R^{1b} is hydroxyethyl; R^{1a} is hydroxyethyl, and R^{1b} is 2-hydroxy-2-propyl; R^{1a} is hydroxyethyl, and R^{1b} is 3-hydroxy-2-propyl; R^{1a} is hydroxyethyl, and R^{1b} is 1-hydroxy-1-propyl; R^{1a} is hydroxyethyl, and R^{1b} is 2-hydroxy-1-propyl; R^{1a} is hydroxyethyl, and R^{1b} is 3-hydroxy-1-propyl; R^{1a} is hydroxyethyl, and R^{1b} is hydroxybutyl; R^{1a} is hydroxyethyl, and R^{1b} is hydroxypentyl; R^{1a} is hydroxyethyl, and R^{1b} is hydroxyhexyl; R^{1a} is 2-hydroxy-2-propyl, and R^{1b} is hydroxymethyl; R^{1a} is 2-hydroxy-2-propyl, and R^{1b} is hydroxyethyl; R^{1a} is 2-hydroxy-2-propyl, and R^{1b} is 2-hydroxy-2-propyl; R^{1a} is 2-hydroxy-2-propyl, and R^{1b} is 3-hydroxy-2-propyl; R^{1a} is 2-hydroxy-2-propyl, and R^{1b} is 1-hydroxy-1-propyl; R^{1a} is 2-hydroxy-2-propyl, and R^{1b} is 2-hydroxy-1-propyl; R^{1a} is 2-hydroxy-2-propyl, and R^{1b} is 3-hydroxy-1-propyl; R^{1a} is 2-hydroxy-2-propyl, and R^{1b} is hydroxybutyl; R^{1a} is 2-hydroxy-2-propyl, and R^{1b} is hydroxypentyl; R^{1a} is 2-hydroxy-2-propyl, and R^{1b} is hydroxyhexyl; R^{1b} is hydroxymethyl, and R^{1a} is hydroxymethyl; R^{1b} is hydroxymethyl, and R^{1a} is hydroxy ethyl; R^{1b} is hydroxymethyl, and R^{1a} is 2-hydroxy-2-propyl; R^{1b} is hydroxymethyl, and R^{1a} is 3-hydroxy-2-propyl; R^{1b} is hydroxymethyl, and R^{1a} is 1-hydroxy-1-propyl; R^{1b} is hydroxymethyl, and R^{1a} is 2-hydroxy-1-propyl; R^{1b} is hydroxymethyl, and R^{1a} is 3-hydroxy-1-propyl; R^{1b} is hydroxymethyl, and R^{1a} is hydroxybutyl; R^{1b} is hydroxymethyl, and R^{1a} is hydroxypentyl; R^{1b} is hydroxymethyl, and R^{1a} is hydroxyhexyl; R^{1b} is hydroxy ethyl, and R^{1a} is hydroxymethyl; R^{1b} is hydroxy ethyl, and R^{1a} is hydroxyethyl; R^{1b} is hydroxyethyl, and R^{1a} is 2-hydroxy-2-propyl; R^{1b} is hydroxyethyl, and R^{1a} is 3-hydroxy-2-propyl; R^{1b} is hydroxyethyl, and R^{1a} is 1-hydroxy-1-propyl; R^{1b} is hydroxyethyl, and R^{1a} is 2-hydroxy-1-propyl; R^{1b} is hydroxyethyl, and R^{1a} is 3-hydroxy-1-propyl; R^{1b} is hydroxyethyl, and R^{1a} is hydroxybutyl; R^{1b} is hydroxyethyl, and R^{1a} is hydroxypentyl; R^{1b} is hydroxyethyl, and R^{1a} is hydroxyhexyl; R^{1b} is 2-hydroxy-2-propyl, and R^{1a} is hydroxymethyl; R^{1b} is 2-hydroxy-2-propyl, and R^{1a} is hydroxyethyl; R^{1b} is 2-hydroxy-2-propyl, and R^{1a} is 2-hydroxy-2-propyl; R^{1b} is 2-hydroxy-2-propyl, and R^{1a} is 3-hydroxy-2-propyl; R^{1b} is 2-hydroxy-2-propyl, and R^{1a} is 1-hydroxy-1-propyl; R^{1b} is 2-hydroxy-2-propyl, and R^{1a} is 2-hydroxy-1-propyl; R^{1b} is 2-hydroxy-2-propyl, and R^{1a} is 3-hydroxy-1-propyl; R^{1b} is 2-hydroxy-2-propyl, and R^{1a} is hydroxybutyl; R^{1b} is 2-hydroxy-2-propyl, and R^{1a} is hydroxypentyl; and R^{1b} is 2-hydroxy-2-propyl, and R^{1a} is hydroxyhexyl.

In some embodiments of the compound of formula AA, the substituted ring A is

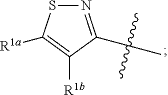

and R^{1a} and R^{1b} are one of the following combinations:
R^{1a} is hydroxymethyl, and R^{1b} is hydroxymethyl; R^{1a} is hydroxymethyl, and R^{1b} is hydroxyethyl; R^{1a} is hydroxymethyl, and R^{1b} is 2-hydroxy-2-propyl; R^{1a} is hydroxymethyl, and R^{1b} is 3-hydroxy-2-propyl; R^{1a} is hydroxymethyl, and R^{1b} is 1-hydroxy-1-propyl; R^{1a} is hydroxymethyl, and R^{1b} is 2-hydroxy-1-propyl; R^{1a} is hydroxymethyl, and R^{1b} is 3-hydroxy-1-propyl; R^{1a} is hydroxymethyl, and R^{1b} is hydroxybutyl; R^{1a} is hydroxymethyl, and R^{1b} is hydroxypentyl; R^{1a} is hydroxymethyl, and R^{1b} is hydroxyhexyl; R^{1a} is hydroxyethyl, and R^{1b} is hydroxymethyl; R^{1a} is hydroxyethyl, and R^{1b} is hydroxyethyl; R^{1a} is hydroxyethyl, and R^{1b} is 2-hydroxy-2-propyl; R^{1a} is hydroxy ethyl, and R^{1b} is 3-hydroxy-2-propyl; R^{1a} is hydroxyethyl, and R^{1b} is 1-hydroxy-1-propyl; R^{1a} is hydroxyethyl, and R^{1b} is 2-hydroxy-1-propyl; R^{1a} is hydroxyethyl, and R^{1b} is 3-hydroxy-1-propyl; R^{1a} is hydroxyethyl, and R^{1b} is hydroxybutyl; R^{1a} is hydroxyethyl, and R^{1b} is hydroxypentyl; R^{1a} is hydroxyethyl, and R^{1b} is hydroxyhexyl; R^{1a} is 2-hydroxy-2-propyl, and R^{1b} is hydroxymethyl; R^{1a} is 2-hydroxy-2-propyl, and R^{1b} is hydroxyethyl; R^{1a} is 2-hydroxy-2-propyl, and R^{1b} is 2-hydroxy-2-propyl; R^{1a} is 2-hydroxy-2-propyl, and R^{1b} is 3-hydroxy-2-propyl; R^{1a} is 2-hydroxy-2-propyl, and R^{1b} is 1-hydroxy-1-propyl; R^{1a} is 2-hydroxy-2-propyl, and R^{1b} is 2-hydroxy-1-propyl; R^{1a} is 2-hydroxy-2-propyl, and R^{1b} is 3-hydroxy-1-propyl; R^{1a} is 2-hydroxy-2-propyl, and R^{1b} is hydroxybutyl; R^{1a} is 2-hydroxy-2-propyl, and R^{1b} is hydroxypentyl; R^{1a} is 2-hydroxy-2-propyl, and R^{1b} is hydroxyhexyl; R^{1b} is hydroxymethyl, and R^{1a} is hydroxymethyl; R^{1b} is hydroxymethyl, and R^{1a} is hydroxyethyl; R^{1b} is hydroxymethyl, and R^{1a} is 2-hydroxy-2-propyl; R^{1b} is hydroxymethyl, and R^{1a} is 3-hydroxy-2-propyl; R^{1b} is hydroxymethyl, and R^{1a} is 1-hydroxy-1-propyl; R^{1b} is hydroxymethyl, and R^{1a} is 2-hydroxy-1-propyl; R^{1b} is hydroxymethyl, and R^{1a} is 3-hydroxy-1-propyl; R^{1b} is hydroxymethyl, and R^{1a} is hydroxybutyl; R^{1b} is hydroxymethyl, and R^{1a} is hydroxypentyl; R^{1b} is hydroxymethyl, and R^{1a} is hydroxyhexyl; R^{1b} is hydroxyethyl, and R^{1a} is hydroxymethyl; R^{1b} is hydroxyethyl, and R^{1a} is hydroxyethyl; R^{1b} is hydroxyethyl, and R^{1a} is 2-hydroxy-2-propyl; R^{1b} is hydroxyethyl, and R^{1a} is 3-hydroxy-2-propyl; R^{1b} is hydroxyethyl, and R^{1a} is 1-hydroxy-1-propyl; R^{1b} is hydroxyethyl, and R^{1a} is 2-hydroxy-1-propyl; R^{1b} is hydroxyethyl, and R^{1a} is 3-hydroxy-1-propyl; R^{1b} is hydroxyethyl, and R^{1a} is hydroxybutyl; R^{1b} is hydroxyethyl, and R^{1a} is hydroxypentyl; R^{1b} is hydroxyethyl, and R^{1a} is hydroxyhexyl; R^{1b} is 2-hydroxy-2-propyl, and R^{1a} is hydroxymethyl; R^{1b} is 2-hydroxy-2-propyl, and R^{1a} is hydroxyethyl; R^{1b} is 2-hydroxy-2-propyl, and R^{1a} is 2-hydroxy-2-propyl; R^{1b} is 2-hydroxy-2-propyl, and R^{1a} is 3-hydroxy-2-propyl; R^{1b} is 2-hydroxy-2-propyl, and R^{1a} is 1-hydroxy-1-propyl; R^{1b} is 2-hydroxy-2-propyl, and R^{1a} is 2-hydroxy-1-propyl; R^{1b} is 2-hydroxy-2-propyl, and R^{1a} is 3-hydroxy-1-propyl; R^{1b} is 2-hydroxy-2-propyl, and R^{1a} is hydroxybutyl; R^{1b} is 2-hydroxy-2-propyl, and R^{1a} is hydroxypentyl; and R^{1b} is 2-hydroxy-2-propyl, and R^{1a} is hydroxyhexyl.

In some embodiments of the compound of formula AA,

the substituted ring A is

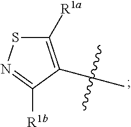

and R^{1a} and R^{1b} are one of the following combinations:
R^{1a} is hydroxymethyl, and R^{1b} is hydroxymethyl; R^{1a} is hydroxymethyl, and R^{1b} is hydroxy ethyl; R^{1a} is hydroxymethyl, and R^{1b} is 2-hydroxy-2-propyl; R^{1a} is hydroxymethyl, and R^{1b} is 3-hydroxy-2-propyl; R^{1a} is hydroxymethyl, and R^{1b} is 1-hydroxy-1-propyl; R^{1a} is hydroxymethyl, and R^{1b} is 2-hydroxy-1-propyl; R^{1a} is hydroxymethyl, and R^{1b} is 3-hydroxy-1-propyl; R^{1a} is hydroxymethyl, and R^{1b} is hydroxybutyl; R^{1a} is hydroxymethyl, and R^{1b} is hydroxypentyl; R^{1a} is hydroxymethyl, and R^{1b} is hydroxyhexyl; R^{1a} is hydroxy ethyl, and R^{1b} is hydroxymethyl; R^{1a} is hydroxy ethyl, and R^{1b} is hydroxy ethyl; R^{1a} is hydroxy ethyl, and R^{1b} is 2-hydroxy-2-propyl; R^{1a} is hydroxy ethyl, and R^{1b} is 3-hydroxy-2-propyl; R^{1a} is hydroxyethyl, and R^{1b} is 1-hydroxy-1-propyl; R^{1a} is hydroxyethyl, and R^{1b} is 2-hydroxy-1-propyl; R^{1a} is hydroxyethyl, and R^{1b} is 3-hydroxy-1-propyl; R^{1a} is hydroxyethyl, and R^{1b} is hydroxybutyl; R^{1a} is hydroxyethyl, and R^{1b} is hydroxypentyl; R^{1a} is hydroxyethyl, and R^{1b} is hydroxyhexyl; R^{1a} is 2-hydroxy-2-propyl, and R^{1b} is hydroxymethyl; R^{1a} is 2-hydroxy-2-propyl, and R^{1b} is hydroxyethyl; R^{1a} is 2-hydroxy-2-propyl, and R^{1b} is 2-hydroxy-2-propyl; R^{1a} is 2-hydroxy-2-propyl, and R^{1b} is 3-hydroxy-2-propyl; R^{1a} is 2-hydroxy-2-propyl, and R^{1b} is 1-hydroxy-1-propyl; R^{1a} is 2-hydroxy-2-propyl, and R^{1b} is 2-hydroxy-1-propyl; R^{1a} is 2-hydroxy-2-propyl, and R^{1b} is 3-hydroxy-1-propyl; R^{1a} is 2-hydroxy-2-propyl, and R^{1b} is hydroxybutyl; R^{1a} is 2-hydroxy-2-propyl, and R^{1b} is hydroxypentyl; R^{1a} is 2-hydroxy-2-propyl, and R^{1b} is hydroxyhexyl; R^{1b} is hydroxymethyl, and R^{1a} is hydroxymethyl; R^{1b} is hydroxymethyl, and R^{1a} is hydroxyethyl; R^{1b} is hydroxymethyl, and R^{1a} is 2-hydroxy-2-propyl; R^{1b} is hydroxymethyl, and R^{1a} is 3-hydroxy-2-propyl; R^{1b} is hydroxymethyl, and R^{1a} is 1-hydroxy-1-propyl; R^{1b} is hydroxymethyl, and R^{1a} is 2-hydroxy-1-propyl; R^{1b} is hydroxymethyl, and R^{1a} is 3-hydroxy-1-propyl; R^{1b} is hydroxymethyl, and R^{1a} is hydroxybutyl; R^{1b} is hydroxymethyl, and R^{1a} is hydroxypentyl; R^{1b} is hydroxymethyl, and R^{1a} is hydroxyhexyl; R^{1b} is hydroxyethyl, and R^{1a} is hydroxymethyl; R^{1b} is hydroxyethyl, and R^{1a} is hydroxyethyl; R^{1b} is hydroxyethyl, and R^{1a} is 2-hydroxy-2-propyl; R^{1b} is hydroxyethyl, and R^{1a} is 3-hydroxy-2-propyl; R^{1b} is hydroxyethyl, and R^{1a} is 1-hydroxy-1-propyl; R^{1b} is hydroxy ethyl, and R^{1a} is 2-hydroxy-1-propyl; R^{1b} is hydroxy ethyl, and R^{1a} is 3-hydroxy-1-propyl; R^{1b} is hydroxy ethyl, and R^{1a} is hydroxybutyl; R^{1b} is hydroxy ethyl, and R^{1a} is hydroxypentyl; R^{1b} is hydroxyethyl, and R^{1a} is hydroxyhexyl; R^{1b} is 2-hydroxy-2-propyl, and R^{1a} is hydroxymethyl; R^{1b} is 2-hydroxy-2-propyl, and R^{1a} is hydroxyethyl; R^{1b} is 2-hydroxy-2-propyl, and R^{1a} is 2-hydroxy-2-propyl; R^{1b} is 2-hydroxy-2-propyl, and R^{1a} is 3-hydroxy-2-propyl; R^{1b} is 2-hydroxy-2-propyl, and R^{1a} is 1-hydroxy-1-propyl; R^{1b} is 2-hydroxy-2-propyl, and R^{1a} is 2-hydroxy-1-propyl; R^{1b} is 2-hydroxy-2-propyl, and R^{1a} is 3-hydroxy-1-propyl; R^{1b} is 2-hydroxy-2-propyl, and R^{1a} is hydroxybutyl; R^{1b} is 2-hydroxy-2-propyl, and R^{1a} is hydroxypentyl; and R^{1b} is 2-hydroxy-2-propyl, and R^{1a} is hydroxyhexyl.

In some embodiments of the compound of formula AA,

the substituted ring A is

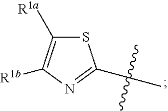

R^{1a} and R^{1b} are one of the following combinations:
R^{1a} is C₁-C₆ alkyl substituted with one or more hydroxy, and R^{1b} is C₁-C₆ alkyl substituted with one or more hydroxy; R^{1a} is C₁-C₆ alkyl substituted with one or more hydroxy, and R^{1b} is —SO₂NR¹¹R¹²; R^{1a} is C₁-C₆ alkyl substituted with one or more hydroxy, and R^{1b} is —SO₂R¹³; R^{1a} is C₁-C₆ alkyl substituted with one or more hydroxy, and R^{1b} is —CONR¹¹R¹²; R^{1a} is C₁-C₆ alkyl substituted with one or more hydroxy, and R^{1b} is —OR¹¹; R^{1a} is C₁-C₆ alkyl substituted with one or more hydroxy, and R^{1b} is —COR¹³; R^{1a} is C₁-C₆ alkyl substituted with one or more hydroxy, and R^{1b} is —CO₂R¹³; R^{1a} is C₁-C₆ alkyl substituted with one or more hydroxy, and R^{1b} is —NR¹³CONR¹¹R¹²; R^{1a} is C₁-C₆ alkyl substituted with one or more hydroxy, and R^{1b} is —CR¹¹R¹²CN; R^{1a} is C₁-C₆ alkyl substituted with one or more hydroxy, and R^{1b} is —NR¹¹SO₂R¹³; R^{1a} is C₁-C₆ alkyl substituted with one or more hydroxy, and R^{1b} is —NR¹¹CONR¹¹R¹²; R^{1a} is C₁-C₆ alkyl substituted with one or more hydroxy, and R^{1b} is —CN; R^{1a} is C₁-C₆ alkyl substituted with one or more hydroxy, and R^{1b} is —NR¹¹COR¹²; R^{1a} is C₁-C₆ alkyl substituted with one or more hydroxy, and R^{1b} is —CR¹¹R¹²NR¹¹R¹²; R^{1a} is C₁-C₆ alkyl substituted with one or more —OSi(R¹³)₃, and R^{1b} is C₁-C₆ alkyl substituted with one or more hydroxy; R^{1a} is C₁-C₆ alkyl substituted with one or more —OSi(R¹³)₃, and R^{1b} is —SO₂NR¹¹R¹²; R^{1a} is C₁-C₆ alkyl substituted with one or more —OSi(R¹³)₃, and R^{1b} is —SO₂R¹³; R^{1a} is C₁-C₆ alkyl substituted with one or more —OSi(R¹³)₃, and R^{1b} is —CONR¹¹R¹²; R^{1a} is C₁-C₆ alkyl substituted with one or more —OSi(R¹³)₃, and R^{1b} is —OR¹¹; R^{1a} is C₁-C₆ alkyl substituted with one or more —OSi(R¹³)₃, and R^{1b} is —COR¹³; R^{1a} is C₁-C₆ alkyl substituted with one or more —OSi(R¹³)₃, and R^{1b} is —CO₂R¹³; R^{1a} is C₁-C₆ alkyl substituted with one or more —OSi(R¹³)₃, and R^{1b} is —NR¹³CONR¹¹R¹²; R^{1a} is C₁-C₆ alkyl substituted with one or more —OSi(R¹³)₃, and R^{1b} is —CR¹¹R¹²CN; R^{1a} is C₁-C₆ alkyl substituted with one or more —OSi(R¹³)₃, and R^{1b} is —NR¹¹SO₂R¹³; R^{1a} is C₁-C₆ alkyl substituted with one or more —OSi(R¹³)₃, and R^{1b} is —NR¹¹CONR¹¹R¹²; R^{1a} is C₁-C₆ alkyl substituted with one or more —OSi(R¹³)₃, and R^{1b} is —CN; R^{1a} is C₁-C₆ alkyl substituted with one or more —OSi(R¹³)₃, and R^{1b} is —NR¹¹COR¹²; R^{1a} is C₁-C₆ alkyl substituted with one or more —OSi(R¹³)₃, and R^{1b} is —CR¹¹R¹²NR¹¹R¹²; R^{1a} is —SO₂NR¹¹R¹², and R^{1b} is C₁-C₆ alkyl substituted with one or more hydroxy; R^{1a} is —SO₂NR¹¹R¹², and R^{1b} is —SO₂NR¹¹R¹²; R^{1a} is —SO₂NR¹¹R¹², and R^{1b} is —SO₂R¹³; R^{1a} is —SO₂NR¹¹R¹², and R^{1b} is —CONR¹¹R¹²; R^{1a} is —SO₂NR¹¹R¹², and R^{1b} is —OR¹¹; R^{1a} is —SO₂NR¹¹R¹², and R^{1b} is —COR¹³; R^{1a} is —SO₂NR¹¹R¹², and R^{1b} is —CO₂R¹³; R^{1a} is —SO₂NR¹¹R¹², and R^{1b} is —NR¹³CONR¹¹R¹²; R^{1a} is —SO₂NR¹¹R¹², and R^{1b} is —CR¹¹R¹²CN; R^{1a} is —SO₂NR¹¹R¹², and R^{1b} is —NR¹¹SO₂R¹³; R^{1a} is —SO₂NR¹¹R¹², and R^{1b} is —CR¹¹R¹²NR¹¹R¹²; R^{1a} is —SO₂NR¹¹R¹², and R^{1b} is —CN; R^{1a} is —SO₂NR¹¹R¹², and R^{1b} is —NR¹¹CONR¹¹R¹²; R^{1a} is —SO₂NR¹¹R¹², and R^{1b} is —NR¹¹COR¹²; R^{1a} is —CR¹¹R¹²NR¹¹R¹², and R^{1b} is C₁-C₆ alkyl substituted with one or more hydroxy; R^{1a} is —CR¹¹R¹²NR¹¹R¹², and R^{1b} is —SO₂NR¹¹R¹²; R^{1a} is —CR¹¹R¹²NR¹¹R¹², and R^{1b} is —SO₂R¹³; R^{1a} is —CR¹¹R¹²NR¹¹R¹², and R^{1b} is —CONR¹¹R¹²; R^{1a} is —CR¹¹R¹²NR¹¹R¹², and R^{1b} is —OR¹¹; R^{1a} is —CR¹¹R¹²NR¹¹R¹², and R^{1b} is —COR¹³; R^{1a} is —CR¹¹R¹²NR¹¹R¹², and R^{1b} is —CO₂R¹³; R^{1a} is —CR¹¹R¹²NR¹¹R¹², and R^{1b} is —NR¹³CONR¹¹R¹²; R^{1a} is —CR¹¹R¹²NR¹¹R¹², and R^{1b} is —CR¹¹R¹²CN; R^{1a} is —CR¹¹R¹²NR¹¹R¹², and R^{1b} is —NR¹¹SO₂R¹³; R^{1a} is —CR¹¹R¹²NR¹¹R¹², and R^{1b} is —CR¹¹R¹²NR¹¹R¹²; R^{1a} is —CR¹¹R¹²NR¹¹R¹², and R^{1b} is —CN; R^{1a} is —CR¹¹R¹²NR¹¹R¹², and R^{1b} is —NR¹¹CONR¹¹R¹²; and R^{1a} is —CR¹¹R¹²NR¹¹R¹², and R^{1b} is —NR¹¹COR¹².

In some embodiments of the compound of formula AA,

the substituted ring A is

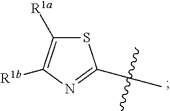

R^{1a} and R^{1b} are one of the following combinations:

In some embodiments, R^{1a} is 2-hydroxy-2-propyl, and R^{1b} is —OMe; In some embodiments, R^{1a} is 2-hydroxy-2-propyl, and R^{1b} is —OH; In some embodiments, R^{1a} is 2-hydroxy-2-propyl, and R^{1b} is —CO₂Me; In some embodiments, R^{1a} is 2-hydroxy-2-propyl, and R^{1b} is hydroxymethyl; In some embodiments, R^{1a} is 2-hydroxy-2-propyl, and R^{1b} is hydroxy ethyl; In some embodiments, R^{1a} is 2-hydroxy-2-propyl, and R^{1b} is 2-hydroxy-2-propyl; In some embodiments, R^{1a} is 2-hydroxy-2-propyl, and R^{1b} is —SO₂NHCH₂CH₂OH; In some embodiments, R^{1a} is 2-hydroxy-2-propyl, and R^{1b} is —SO₂Me; In some embodiments, R^{1a} is 2-hydroxy-2-propyl, and R^{1b} is CONHMe; In some embodiments, R^{1a} is 2-hydroxy-2-propyl, and R^{1b} is cyanomethyl; In some embodiments, R^{1a} is 2-hydroxy-2-propyl, and R^{1b} is —CN; In some embodiments, R^{1a} is 2-hydroxy-2-propyl, and R^{1b} is dimethylaminomethyl; In some embodiments, R^{1a} is dimethylaminomethyl, and R^{1b} is —OMe; In some embodiments, R^{1a} is dimethylaminomethyl, and R^{1b} is —OH; In some embodiments, R^{1a} is dimethylaminomethyl, and R^{1b} is —CO₂Me; In some embodiments, R^{1a} is dimethylaminomethyl, and R^{1b} is hydroxymethyl; In some embodiments, R^{1a} is dimethylaminomethyl, and R^{1b} is hydroxy ethyl; In some embodiments, R^{1a} is dimethylaminomethyl, and R^{1b} is 2-hydroxy-2-propyl; In some embodiments, R^{1a} is dimethylaminomethyl, and R^{1b} is —SO₂NHCH₂CH₂OH; In some embodiments, R^{1a} is dimethylaminomethyl, and R^{1b} is —SO₂Me; In some embodiments, R^{1a} is dimethylaminomethyl, and R^{1b} is CONHMe; In some embodiments, R^{1a} is dimethylaminomethyl, and R^{1b} is cyanomethyl; In some embodiments, R^{1a} is dimethylaminomethyl, and R^{1b} is dimethylaminomethyl; In some embodiments, R^{1a} is dimethylaminomethyl, and R^{1b} is CN. In some embodiments, R^{1a} is —SO₂NHMe, and R^{1b} is —OMe; In some embodiments, R^{1a} is —SO₂NHMe, and R^{1b} is —OH; In some embodiments, R^{1a} is —SO₂NHMe, and R^{1b} is —CO₂Me; In some embodiments, R^{1a} is —SO₂NHMe, and R^{1b} is hydroxymethyl; In some embodiments, R^{1a} is —SO₂NHMe, and R^{1b} is hydroxyethyl; In some embodiments, R^{1a} is —SO₂NHMe, and R^{1b} is 2-hydroxy-2-propyl; In some embodiments, R^{1a} is —SO₂NHMe, and R^{1b} is —SO₂NHCH₂CH₂OH; In some embodiments, R^{1a} is —SO₂NHMe, and R^{1b} is —SO₂Me; In some embodiments, R^{1a} is —SO₂NHMe, and R^{1b} is CONHMe; In some embodiments, R^{1a} is —SO₂NHMe, and R^{1b} is dimethylaminomethyl; In some embodiments, R^{1a} is —SO₂NHMe, and R^{1b} is —CN; In some embodiments, R^{1a} is —SO₂NHMe, and R^{1b} is cyanomethyl; and In some embodiments, R^{1a} is C₁-C₄ alkyl substituted with one —OSi(Me)₂tBu, and R^{1b} is —CO₂Me.

In some embodiments of the compound of formula AA,

the substituted ring A is

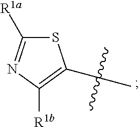

R^{1a} and R^{1b} are one of the following combinations:
R^{1a} is C₁-C₆ alkyl substituted with one or more hydroxy, and R^{1b} is C₁-C₆ alkyl substituted with one or more hydroxy; R^{1a} is C₁-C₆ alkyl substituted with one or more hydroxy, and R^{1b} is —SO₂NR¹¹R¹²; R^{1a} is C₁-C₆ alkyl substituted with one or more hydroxy, and R^{1b} is —SO₂R¹³; R^{1a} is C₁-C₆ alkyl substituted with one or more hydroxy, and R^{1b} is —CONR¹¹R¹²; R^{1a} is C₁-C₆ alkyl substituted with one or more hydroxy, and R^{1b} is —OR¹¹; R^{1a} is C₁-C₆ alkyl substituted with one or more hydroxy, and R^{1b} is —COR¹³; R^{1a} is C₁-C₆ alkyl substituted with one or more hydroxy, and R^{1b} is —CO₂R¹³; R^{1a} is C₁-C₆ alkyl substituted with one or more hydroxy, and R^{1b} is —NR¹³CONR¹¹R¹²; R^{1a} is C₁-C₆ alkyl substituted with one or more hydroxy, and R^{1b} is —CR¹¹R¹²CN; R^{1a} is C₁-C₆ alkyl substituted with one or more hydroxy, and R^{1b} is —NR¹¹SO₂R¹³; R^{1a} is C₁-C₆ alkyl substituted with one or more hydroxy, and R^{1b} is —NR¹¹CONR¹¹R¹²; R^{1a} is C₁-C₆ alkyl substituted with one or more hydroxy, and R^{1b} is —CN; R^{1a} is C₁-C₆ alkyl substituted with one or more hydroxy, and R^{1b} is —NR¹¹COR¹²; R^{1a} is C₁-C₆ alkyl substituted with one or more hydroxy, and R^{1b} is —CR¹¹R¹²NR¹¹R¹²; R^{1a} is C₁-C₆ alkyl substituted with one or more —OSi(R¹³)₃, and R^{1b} is C₁-C₆ alkyl substituted with one or more hydroxy; R^{1a} is C₁-C₆ alkyl substituted with one or more —OSi(R¹³)₃, and R^{1b} is —SO₂NR¹¹R¹²; R^{1a} is C₁-C₆ alkyl substituted with one or more —OSi(R¹³)₃, and R^{1b} is —SO₂R¹³; R^{1a} is C₁-C₆ alkyl substituted with one or more —OSi(R¹³)₃, and R^{1b} is —CONR¹¹R¹²; R^{1a} is C₁-C₆ alkyl substituted with one or more —OSi(R¹³)₃, and R^{1b} is —CN; R^{1a} is C₁-C₆ alkyl substituted with one or more —OSi(R¹³)₃, and R^{1b} is —OR¹¹; R^{1a} is C₁-C₆ alkyl substituted with one or more —OSi(R¹³)₃, and R^{1b} is —COR¹³; R^{1a} is C₁-C₆ alkyl substituted with one or more —OSi(R¹³)₃, and R^{1b} is —CO₂R¹³; R^{1a} is C₁-C₆ alkyl substituted with one or more —OSi(R¹³)₃, and R^{1b} is —NR¹³CONR¹¹R¹²; R^{1a} is C₁-C₆ alkyl substituted with one or more —OSi(R¹³)₃, and R^{1b} is —CR¹¹R¹²CN; R^{1a} is C₁-C₆ alkyl substituted with one or more —OSi(R¹³)₃, and R^{1b} is —NR¹¹SO₂R¹³; R^{1a} is C₁-C₆ alkyl substituted with one or more —OSi(R¹³)₃, and R^{1b} is —NR¹¹CONR¹¹R¹²; R^{1a} is C₁-C₆ alkyl substituted with one or more —OSi(R¹³)₃, and R^{1b} is —NR¹¹COR¹²; R^{1a} is C₁-C₆ alkyl substituted with one or more —OSi(R¹³)₃, and R^{1b} is —CR¹¹R¹²NR¹¹R¹²; R^{1a} is —SO₂NR¹¹R¹², and R^{1b} is C₁-C₆ alkyl substituted with one or more hydroxy; R^{1a} is —SO₂NR¹¹R¹², and R^{1b} is —SO₂NR¹¹R¹²; R^{1a} is —SO₂NR¹¹R¹², and R^{1b} is —SO₂R¹³; R^{1a} is —SO₂NR¹¹R¹², and R^{1b} is —CONR¹¹R¹²; R^{1a} is —SO₂NR¹¹R¹², and R^{1b} is —OR¹¹; R^{1a} is —SO₂NR¹¹R¹², and R^{1b} is —COR¹³; R^{1a} is —SO₂NR¹¹R¹², and R^{1b} is —CO₂R¹³; R^{1a} is —SO₂NR¹¹R¹², and R^{1b} is —NR¹³CONR¹¹R¹²; R^{1a} is —SO₂NR¹¹R¹², and R^{1b} is —CR¹¹R¹²CN; R^{1a} is —SO₂NR¹¹R¹², and R^{1b} is —NR¹¹SO₂R¹³; R^{1a} is —SO₂NR¹¹R¹², and R^{1b} is —CR¹¹R¹²NR¹¹R¹²; R^{1a} is —SO₂NR¹¹R¹², and R^{1b} is —CN; R^{1a} is —SO₂NR¹¹R¹², and R^{1b} is —NR¹¹CONR¹¹R¹²; R^{1a} is —SO₂NR¹¹R¹², and R^{1b} is —NR¹¹COR¹²; R^{1a} is —CR¹¹R¹²NR¹¹R¹², and R^{1b} is C₁-C₆ alkyl substituted with one or more hydroxy; R^{1a} is —CR¹¹R¹²NR¹¹R¹², and R^{1b} is —SO₂NR¹¹R¹²; R^{1a} is —CR¹¹R¹²NR¹¹R¹², and R^{1b} is —SO₂R¹³; R^{1a} is —CR¹¹R¹²NR¹¹R¹², and R^{1b} is —CONR¹¹R¹²; R^{1a} is —CR¹¹R¹²NR¹¹R¹², and R^{1b} is —OR¹¹; R^{1a} is —CR¹¹R¹²NR¹¹R¹², and R^{1b} is —COR¹³; R^{1a} is —CR¹¹R¹²NR¹¹R¹², and R^{1b} is —CO₂R¹³; R^{1a} is —CR¹¹R¹²NR¹¹R¹², and R^{1b} is —NR¹³CONR¹¹R¹²; R^{1a} is —CR¹¹R¹²NR¹¹R¹², and R^{1b} is —CR¹¹R¹²CN; R^{1a} is —CR¹¹R¹²NR¹¹R¹², and R^{1b} is —NR¹¹SO₂R¹³; R^{1a} is —CR¹¹R¹²NR¹¹R¹², and R^{1b} is —CR¹¹R¹²NR¹¹R¹²; R^{1a} is —CR¹¹R¹²NR¹¹R¹², and R^{1b} is —CN; R^{1a} is —CR¹¹R¹²NR¹¹R¹², and R^{1b} is —NR¹¹CONR¹¹R¹²; and R^{1a} is —CR¹¹R¹²NR¹¹R¹², and R^{1b} is —NR¹¹COR¹².

In some embodiments of the compound of formula AA,

the substituted ring A is

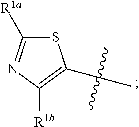

R^{1a} and R^{1b} are one of the following combinations:

In some embodiments, R^{1a} is 2-hydroxy-2-propyl, and R^{1b} is —OMe; In some embodiments, R^{1a} is 2-hydroxy-2-propyl, and R^{1b} is —OH; In some embodiments, R^{1a} is 2-hydroxy-2-propyl, and R^{1b} is —CO₂Me; In some embodiments, R^{1a} is 2-hydroxy-2-propyl, and R^{1b} is hydroxymethyl; In some embodiments, R^{1a} is 2-hydroxy-2-propyl, and R^{1b} is hydroxy ethyl; In some embodiments, R^{1a} is 2-hydroxy-2-propyl, and R^{1b} is 2-hydroxy-2-propyl; In some embodiments, R^{1a} is 2-hydroxy-2-propyl, and R^{1b} is —SO₂NHCH₂CH₂OH; In some embodiments, R^{1a} is 2-hydroxy-2-propyl, and R^{1b} is —SO₂Me; In some embodiments, R^{1a} is 2-hydroxy-2-propyl, and R^{1b} is CONHMe; In some embodiments, R^{1a} is 2-hydroxy-2-propyl, and R^{1b} is cyanomethyl; In some embodiments, R^{1a} is 2-hydroxy-2-propyl, and R^{1b} is —CN; In some embodiments, R^{1a} is 2-hydroxy-2-propyl, and R^{1b} is dimethylaminomethyl; In some embodiments, R^{1a} is dimethylaminomethyl, and R^{1b} is —OMe; In some embodiments, R^{1a} is dimethylaminomethyl, and R^{1b} is —OH; In some embodiments, R^{1a} is dimethylaminomethyl, and R^{1b} is —CO₂Me; In some embodiments, R^{1a} is dimethylaminomethyl, and R^{1b} is hydroxymethyl; In some embodiments, R^{1a} is dimethylaminomethyl, and R^{1b} is hydroxy ethyl; In some embodiments, R^{1a} is dimethylaminomethyl, and R^{1b} is 2-hydroxy-2-propyl; In some embodiments, R^{1a} is dimethylaminomethyl, and R^{1b} is —SO₂NHCH₂CH₂OH; In some embodiments, R^{1a} is dimethylaminomethyl, and R^{1b} is —SO₂Me; In some embodiments, R^{1a} is dimethylaminomethyl, and R^{1b} is CONHMe; In some embodiments, R^{1a} is dimethylaminomethyl, and R^{1b} is cyanomethyl; In some embodiments, R^{1a} is dimethylaminomethyl, and R^{1b} is dimethylaminomethyl; In some embodiments, R^{1a} is dimethylaminomethyl, and R^{1b} is CN; In some embodiments, R^{1a} is —SO₂NHMe, and R^{1b} is —OMe; In some embodiments, R^{1a} is —SO₂NHMe, and R^{1b} is —OH; In some embodiments, R^{1a} is —SO₂NHMe, and R^{1b} is —CO₂Me; In some embodiments, R^{1a} is —SO₂NHMe, and R^{1b} is hydroxymethyl; In some embodiments, R^{1a} is —SO₂NHMe, and R^{1b} is hydroxyethyl; In some embodiments, R^{1a} is —SO₂NHMe, and R^{1b} is 2-hydroxy-2-propyl; In some embodiments, R^{1a} is —SO₂NHMe, and R^{1b} is —SO₂NHCH₂CH₂OH; In some embodiments, R^{1a} is —SO₂NHMe, and R^{1b} is —SO₂Me; In some embodiments, R^{1a} is —SO₂NHMe, and R^{1b} is CONHMe; In some embodiments, R^{1a} is —SO₂NHMe, and R^{1b} is dimethylaminomethyl; In some embodiments, R^{1a} is —SO₂NHMe, and R^{1b} is —CN; In some embodiments, R^{1a} is —SO₂NHMe, and R^{1b} is cyanomethyl; and In some embodiments, R^{1a} is C₁-C₄ alkyl substituted with one —OSi(Me)₂tBu, and R^{1b} is —CO₂Me.

In some embodiments of the compound of formula AA,

the substituted ring A is

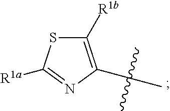

R^{1a} and R^{1b} are one of the following combinations:
R^{1a} is C₁-C₆ alkyl substituted with one or more hydroxy, and R^{1b} is C₁-C₆ alkyl substituted with one or more hydroxy; R^{1a} is C₁-C₆ alkyl substituted with one or more hydroxy, and R^{1b} is —SO₂NR¹¹R¹²; R^{1a} is C₁-C₆ alkyl substituted with one or more hydroxy, and R^{1b} is —SO₂R¹³; R^{1a} is C₁-C₆ alkyl substituted with one or more hydroxy, and R^{1b} is —CONR¹¹R¹²; R^{1a} is C₁-C₆ alkyl substituted with one or more hydroxy, and R^{1b} is —OR¹¹; R^{1a} is C₁-C₆ alkyl substituted with one or more hydroxy, and R^{1b} is —COR¹³; R^{1a} is C₁-C₆ alkyl substituted with one or more hydroxy, and R^{1b} is —CO₂R¹³; R^{1a} is C₁-C₆ alkyl substituted with one or more hydroxy, and R^{1b} is —NR¹³CONR¹¹R¹²; R^{1a} is C₁-C₆ alkyl substituted with one or more hydroxy, and R^{1b} is —CR¹¹R¹²CN; R^{1a} is C₁-C₆ alkyl substituted with one or more hydroxy, and R^{1b} is —NR¹¹SO₂R¹³; R^{1a} is C₁-C₆ alkyl substituted with one or more hydroxy, and R^{1b} is —NR¹¹CONR¹¹R¹²; R^{1a} is C₁-C₆ alkyl substituted with one or more hydroxy, and R^{1b} is —CN; R^{1a} is C₁-C₆ alkyl substituted with one or more hydroxy, and R^{1b} is —NR¹¹COR¹²; R^{1a} is C₁-C₆ alkyl substituted with one or more hydroxy, and R^{1b} is —CR¹¹R¹²NR¹¹R¹²; R^{1a} is C₁-C₆ alkyl substituted with one or more —OSi(R¹³)₃, and R^{1b} is C₁-C₆ alkyl substituted with one or more hydroxy; R^{1a} is C₁-C₆ alkyl substituted with one or more —OSi(R¹³)₃, and R^{1b} is —SO₂NR¹¹R¹²; R^{1a} is C₁-C₆ alkyl substituted with one or more —OSi(R¹³)₃, and R^{1b} is —SO₂R¹³; R^{1a} is C₁-C₆ alkyl substituted with one or more —OSi(R¹³)₃, and R^{1b} is —CONR¹¹R¹²; R^{1a} is C₁-C₆ alkyl substituted with one or more —OSi(R¹³)₃, and R^{1b} is —OR¹¹; R^{1a} is C₁-C₆ alkyl substituted with one or more —OSi(R¹³)₃, and R^{1b} is —COR¹³; R^{1a} is C₁-C₆ alkyl substituted with one or more —OSi(R¹³)₃, and R^{1b} is —CO₂R¹³; R^{1a} is C₁-C₆ alkyl substituted with one or more —OSi(R¹³)₃, and R^{1b} is —NR¹³CONR¹¹R¹²; R^{1a} is C₁-C₆ alkyl substituted with one or more —OSi(R¹³)₃, and R^{1b} is —CR¹¹R¹²CN; R^{1a} is C₁-C₆ alkyl substituted with one or more —OSi(R¹³)₃, and R^{1b} is —NR¹¹SO₂R¹³; R^{1a} is C₁-C₆ alkyl substituted with one or more —OSi(R¹³)₃, and R^{1b} is —NR¹¹CONR¹¹R¹²; R^{1a} is C₁-C₆ alkyl substituted with one or more —OSi(R¹³)₃, and R^{1b} is —CN; R^{1a} is C₁-C₆ alkyl substituted with one or more —OSi(R¹³)₃, and R^{1b} is —NR¹¹COR¹²; R^{1a} is C₁-C₆ alkyl substituted with one or more —OSi(R¹³)₃, and R^{1b} is —CR¹¹R¹²NR¹¹R¹²; R^{1a} is —SO₂NR¹¹R¹², and R^{1b} is C₁-C₆ alkyl substituted with one or more hydroxy; R^{1a} is —SO₂NR¹¹R¹², and R^{1b} is —SO₂NR¹¹R¹²; R^{1a} is —SO₂NR¹¹R¹², and R^{1b} is —SO₂R¹³; R^{1a} is —SO₂NR¹¹R¹², and R^{1b} is —CONR¹¹R¹²; R^{1a} is —SO₂NR¹¹R¹², and R^{1b} is —OR¹¹; R^{1a} is —SO₂NR¹¹R¹², and R^{1b} is —COR¹³; R^{1a} is —SO₂NR¹¹R¹², and R^{1b} is —CO₂R¹³; R^{1a} is —SO₂NR¹¹R¹², and R^{1b} is —NR¹³CONR¹¹R¹²; R^{1a} is —SO₂NR¹¹R¹², and R^{1b} is —CR¹¹R¹²CN; R^{1a} is —SO₂NR¹¹R¹², and R^{1b} is —NR¹¹SO₂R¹³; R^{1a} is —SO₂NR¹¹R¹², and R^{1b} is —CR¹¹R¹²NR¹¹R¹²; R^{1a} is —SO₂NR¹¹R¹², and R^{1b} is —CN; R^{1a} is —SO₂NR¹¹R¹², and R^{1b} is —NR¹¹CONR¹¹R¹²; R^{1a} is —SO₂NR¹¹R¹², and R^{1b} is —NR¹¹COR¹²; R^{1a} is —CR¹¹R¹²NR¹¹R¹², and R^{1b} is C₁-C₆ alkyl substituted with one or more hydroxy; R^{1a} is —CR¹¹R¹²NR¹¹R¹², and R^{1b} is —SO₂NR¹¹R¹²; R^{1a} is —CR¹¹R¹²NR¹¹R¹², and R^{1b} is —SO₂R¹³; R^{1a} is —CR¹¹R¹²NR¹¹R¹², and R^{1b} is —CONR¹¹R¹²; R^{1a} is —CR¹¹R¹²NR¹¹R¹², and R^{1b} is —OR¹¹; R^{1a} is —CR¹¹R¹²NR¹¹R¹², and R^{1b} is —COR¹³; R^{1a} is —CR¹¹R¹²NR¹¹R¹², and R^{1b} is —CO₂R¹³; R^{1a} is —CR¹¹R¹²NR¹¹R¹², and R^{1b} is —NR¹³CONR¹¹R¹²; R^{1a} is —CR¹¹R¹²NR¹¹R¹², and R^{1b} is —CR¹¹R¹²CN; R^{1a} is —CR¹¹R¹²NR¹¹R¹², and R^{1b} is —NR¹¹SO₂R¹³; R^{1a} is —CR¹¹R¹²NR¹¹R¹², and R^{1b} is —CR¹¹R¹²NR¹¹R¹²; R^{1a} is —CR¹¹R¹²NR¹¹R¹², and R^{1b} is —CN; R^{1a} is —CR¹¹R¹²NR¹¹R¹², and R^{1b} is —NR¹¹CONR¹¹R¹²; and R^{1a} is —CR¹¹R¹²NR¹¹R¹², and R^{1b} is —NR¹¹COR¹².

In some embodiments of the compound of formula AA,

the substituted ring A is

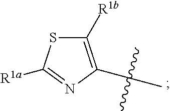

R^{1a} and R^{1b} are one of the following combinations:
In some embodiments, R^{1a} is 2-hydroxy-2-propyl, and R^{1b} is —OMe; In some embodiments, R^{1a} is 2-hydroxy-2-propyl, and R^{1b} is —OH; In some embodiments, R^{1a} is 2-hydroxy-2-propyl, and R^{1b} is —CO₂Me; In some embodiments, R^{1a} is 2-hydroxy-2-propyl, and R^{1b} is hydroxymethyl; In some embodiments, R^{1a} is 2-hydroxy-2-propyl, and R^{1b} is hydroxy ethyl; In some embodiments, R^{1a} is 2-hydroxy-2-propyl, and R^{1b} is 2-hydroxy-2-propyl; In some embodiments, R^{1a} is 2-hydroxy-2-propyl, and R^{1b} is —SO₂NHCH₂CH₂OH; In some embodiments, R^{1a} is 2-hydroxy-2-propyl, and R^{1b} is —SO₂Me; In some embodiments, R^{1a} is 2-hydroxy-2-propyl, and R^{1b} is CONHMe; In some embodiments, R^{1a} is 2-hydroxy-2-propyl, and R^{1b} is cyanomethyl; In some embodiments, R^{1a} is 2-hydroxy-2-propyl, and R^{1b} is —CN; In some embodiments, R^{1a} is 2-hydroxy-2-propyl, and R^{1b} is dimethylaminomethyl; In some embodiments, R^{1a} is dimethylaminomethyl, and R^{1b} is —OMe; In some embodiments, R^{1a} is dimethylaminomethyl, and R^{1b} is —OH; In some embodiments, R^{1a} is dimethylaminomethyl, and R^{1b} is —CO₂Me; In some embodiments, R^{1a} is dimethylaminomethyl, and R^{1b} is hydroxymethyl; In some embodiments, R^{1a} is dimethylaminomethyl, and R^{1b} is hydroxy ethyl; In some embodiments, R^{1a} is dimethylaminomethyl, and R^{1b} is 2-hydroxy-2-propyl; In some embodiments, R^{1a} is dimethylaminomethyl, and R^{1b} is —SO₂NHCH₂CH₂OH; In some embodiments, R^{1a} is dimethylaminomethyl, and R^{1b} is —SO₂Me; In some embodiments, R^{1a} is dimethylaminomethyl, and R^{1b} is CONHMe; In some embodiments, R^{1a} is dimethylaminomethyl, and R^{1b} is cyanomethyl; In some embodiments, R^{1a} is dimethylaminomethyl, and R^{1b} is dimethylaminomethyl; In some embodiments, R^{1a} is dimethylaminomethyl, and R^{1b} is CN. In some embodiments, R^{1a} is —SO₂NHMe, and R^{1b} is —OMe; In some embodiments, R^{1a} is —SO₂NHMe, and R^{1b} is —OH; In some embodiments, R^{1a} is —SO₂NHMe, and R^{1b} is —CO₂Me; In some embodiments, R^{1a} is —SO₂NHMe, and R^{1b} is hydroxymethyl; In some embodiments, R^{1a} is —SO₂NHMe, and R^{1b} is hydroxyethyl; In some embodiments, R^{1a} is —SO₂NHMe, and R^{1b} is 2-hydroxy-2-propyl; In some embodiments, R^{1a} is —SO₂NHMe, and R^{1b} is —SO₂NHCH₂CH₂OH; In some embodiments, R^{1a} is —SO₂NHMe, and R^{1b} is —SO₂Me; In some embodiments, R^{1a} is —SO₂NHMe, and R^{1b} is CONHMe; In some embodiments, R^{1a} is —SO₂NHMe, and R^{1b} is —CN; In some embodiments, R^{1a} is —SO₂NHMe, and R^{1b} is cyanomethyl; and In some embodiments, R^{1a} is —SO₂NHMe, and R^{1b} is dimethylaminomethyl; In some embodiments, R^{1a} is C₁-C₄ alkyl substituted with one —OSi(Me)₂tBu, and R^{1b} is —CO₂Me.

In some embodiments of the compound of formula AA,

the substituted ring A is

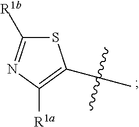

R^{1a} and R^{1b} are one of the following combinations:
R^{1a} is C₁-C₆ alkyl substituted with one or more hydroxy, and R^{1b} is C₁-C₆ alkyl substituted with one or more hydroxy; R^{1a} is C₁-C₆ alkyl substituted with one or more hydroxy, and R^{1b} is —SO₂NR¹¹R¹²; R^{1a} is C₁-C₆ alkyl substituted with one or more hydroxy, and R^{1b} is —SO₂R¹³; R^{1a} is C₁-C₆ alkyl substituted with one or more hydroxy, and R^{1b} is —CONR¹¹R¹²; R^{1a} is C₁-C₆ alkyl substituted with one or more hydroxy, and R^{1b} is —OR¹¹; R^{1a} is C₁-C₆ alkyl substituted with one or more hydroxy, and R^{1b} is —COR¹³; R^{1a} is C₁-C₆ alkyl substituted with one or more hydroxy, and R^{1b} is —CO₂R¹³; R^{1a} is C₁-C₆ alkyl substituted with one or more hydroxy, and R^{1b} is —NR¹³CONR¹¹R¹²; R^{1a} is C₁-C₆ alkyl substituted with one or more hydroxy, and R^{1b} is —CR¹¹R¹²CN; R^{1a} is C₁-C₆ alkyl substituted with one or more hydroxy, and R^{1b} is —NR¹¹SO₂R¹³; R^{1a} is C₁-C₆ alkyl substituted with one or more hydroxy, and R^{1b} is —NR¹¹CONR¹¹R¹²; R^{1a} is C₁-C₆ alkyl substituted with one or more hydroxy, and R^{1b} is —CN; R^{1a} is C₁-C₆ alkyl substituted with one or more hydroxy, and R^{1b} is —NR¹¹COR¹²; R^{1a} is C₁-C₆ alkyl substituted with one or more hydroxy, and R^{1b} is —CR¹¹R¹²NR¹¹R¹²; R^{1a} is C₁-C₆ alkyl substituted with one or more —OSi(R¹³)₃, and R^{1b} is C₁-C₆ alkyl substituted with one or more hydroxy; R^{1a} is C₁-C₆ alkyl substituted with one or more —OSi(R¹³)₃, and R^{1b} is —SO₂NR¹¹R¹²; R^{1a} is C₁-C₆ alkyl substituted with one or more —OSi(R¹³)₃, and R^{1b} is —SO₂R¹³; R^{1a} is C₁-C₆ alkyl substituted with one or more —OSi(R¹³)₃, and R^{1b} is —CONR¹¹R¹²; R^{1a} is C₁-C₆ alkyl substituted with one or more —OSi(R¹³)₃, and R^{1b} is —OR¹¹; R^{1a} is C₁-C₆ alkyl substituted with one or more —OSi(R¹³)₃, and R^{1b} is —COR¹³; R^{1a} is C₁-C₆ alkyl substituted with one or more —OSi(R¹³)₃, and R^{1b} is —CO₂R¹³; R^{1a} is C₁-C₆ alkyl substituted with one or more —OSi(R¹³)₃, and R^{1b} is —NR¹³CONR¹¹R¹²; R^{1a} is C₁-C₆ alkyl substituted with one or more —OSi(R¹³)₃, and R^{1b} is —CR¹¹R¹²CN; R^{1a} is C₁-C₆ alkyl substituted with one or more —OSi(R¹³)₃, and R^{1b} is —NR¹¹SO₂R¹³; R^{1a} is C₁-C₆ alkyl substituted with one or more —OSi(R¹³)₃, and R^{1b} is —NR¹¹CONR¹¹R¹²; R^{1a} is C₁-C₆ alkyl substituted with one or more —OSi(R¹³)₃, and R^{1b} is —CN; R^{1a} is C₁-C₆ alkyl substituted with one or more —OSi(R¹³)₃, and R^{1b} is —NR¹¹COR¹²; R^{1a} is C₁-C₆ alkyl substituted with one or more —OSi(R¹³)₃, and R^{1b} is —CR¹¹R¹²NR¹¹R¹²; R^{1a} is —SO₂NR¹¹R¹², and R^{1b} is C₁-C₆ alkyl substituted with one or more hydroxy; R^{1a} is —SO₂NR¹¹R¹², and R^{1b} is —SO₂NR¹¹R¹²; R^{1a} is —SO₂NR¹¹R¹², and R^{1b} is —SO₂R¹³; R^{1a} is —SO₂NR¹¹R¹², and R^{1b} is —CONR¹¹R¹²; R^{1a} is —SO₂NR¹¹R¹², and R^{1b} is —OR¹¹; R^{1a} is —SO₂NR¹¹R¹², and R^{1b} is —COR¹³; R^{1a} is —SO₂NR¹¹R¹², and R^{1b} is —CO₂R¹³; R^{1a} is —SO₂NR¹¹R¹², and R^{1b} is —NR¹³CONR¹¹R¹²; R^{1a} is —SO₂NR¹¹R¹², and R^{1b} is —CR¹¹R¹²CN; R^{1a} is —SO₂NR¹¹R¹², and R^{1b} is —NR¹¹SO₂R¹³; R^{1a} is —SO₂NR¹¹R¹², and R^{1b} is —CR¹¹R¹²NR¹¹R¹²; R^{1a} is —SO₂NR¹¹R¹², and R^{1b} is —CN; R^{1a} is —SO₂NR¹¹R¹², and R^{1b} is —NR¹¹CONR¹¹R¹²; R^{1a} is —SO₂NR¹¹R¹², and R^{1b} is —NR¹¹COR¹²; R^{1a} is —CR¹¹R¹²NR¹¹R¹², and R^{1b} is C₁-C₆ alkyl substituted with one or more hydroxy; R^{1a} is —CR¹¹R¹²NR¹¹R¹², and R^{1b} is —SO₂NR¹¹R¹²; R^{1a} is —CR¹¹R¹²NR¹¹R¹², and R^{1b} is —SO₂R¹³; R^{1a} is —CR¹¹R¹²NR¹¹R¹², and R^{1b} is —CONR¹¹R¹²; R^{1a} is —CR¹¹R¹²NR¹¹R¹², and R^{1b} is —OR¹¹; R^{1a} is —CR¹¹R¹²NR¹¹R¹², and R^{1b} is —COR¹³; R^{1a} is —CR¹¹R¹²NR¹¹R¹², and R^{1b} is —CO₂R¹³; R^{1a} is —CR¹¹R¹²NR¹¹R¹², and R^{1b} is —NR¹³CONR¹¹R¹²; R^{1a} is —CR¹¹R¹²NR¹¹R¹², and R^{1b} is —CR¹¹R¹²CN; R^{1a} is —CR¹¹R¹²NR¹¹R¹², and R^{1b} is —NR¹¹SO₂R¹³; R^{1a} is —CR¹¹R¹²NR¹¹R¹², and R^{1b} is —CR¹¹R¹²NR¹¹R¹²; R^{1a} is —CR¹¹R¹²NR¹¹R¹², and R^{1b} is —CN; R^{1a} is —CR¹¹R¹²NR¹¹R¹², and R^{1b} is —NR¹¹CONR¹¹R¹²; and R^{1a} is —CR¹¹R¹²NR¹¹R¹², and R^{1b} is —NR¹¹COR¹².

In some embodiments of the compound of formula AA,

the substituted ring A is

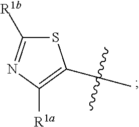

R^{1a} and R^{1b} are one of the following combinations:

In some embodiments, R^{1a} is 2-hydroxy-2-propyl, and R^{1b} is —OMe; In some embodiments, R^{1a} is 2-hydroxy-2-propyl, and R^{1b} is —OH; In some embodiments, R^{1a} is 2-hydroxy-2-propyl, and R^{1b} is —CO₂Me; In some embodiments, R^{1a} is 2-hydroxy-2-propyl, and R^{1b} is hydroxymethyl; In some embodiments, R^{1a} is 2-hydroxy-2-propyl, and R^{1b} is hydroxy ethyl; In some embodiments, R^{1a} is 2-hydroxy-2-propyl, and R^{1b} is 2-hydroxy-2-propyl; In some embodiments, R^{1a} is 2-hydroxy-2-propyl, and R^{1b} is —SO₂NHCH₂CH₂OH; In some embodiments, R^{1a} is 2-hydroxy-2-propyl, and R^{1b} is —SO₂Me; In some embodiments, R^{1a} is 2-hydroxy-2-propyl, and R^{1b} is CONHMe; In some embodiments, R^{1a} is 2-hydroxy-2-propyl, and R^{1b} is cyanomethyl; In some embodiments, R^{1a} is 2-hydroxy-2-propyl, and R^{1b} is —CN; In some embodiments, R^{1a} is 2-hydroxy-2-propyl, and R^{1b} is dimethylaminomethyl; In some embodiments, R^{1a} is dimethylaminomethyl, and R^{1b} is —OMe; In some embodiments, R^{1a} is dimethylaminomethyl, and R^{1b} is —OH; In some embodiments, R^{1a} is dimethylaminomethyl, and R^{1b} is —CO₂Me; In some embodiments, R^{1a} is dimethylaminomethyl, and R^{1b} is hydroxymethyl; In some embodiments, R^{1a} is dimethylaminomethyl, and R^{1b} is hydroxy ethyl; In some embodiments, R^{1a} is dimethylaminomethyl, and R^{1b} is 2-hydroxy-2-propyl; In some embodiments, R^{1a} is dimethylaminomethyl, and R^{1b} is —SO₂NHCH₂CH₂OH; In some embodiments, R^{1a} is dimethylaminomethyl, and R^{1b} is —SO₂Me; In some embodiments, R^{1a} is dimethylaminomethyl, and R^{1b} is CONHMe; In some embodiments, R^{1a} is dimethylaminomethyl, and R^{1b} is cyanomethyl; In some embodiments, R^{1a} is dimethylaminomethyl, and R^{1b} is dimethylaminomethyl; In some embodiments, R^{1a} is dimethylaminomethyl, and R^{1b} is CN. In some embodiments, R^{1a} is —SO₂NHMe, and R^{1b} is —OMe; In some embodiments, R^{1a} is —SO₂NHMe, and R^{1b} is —OH; In some embodiments, R^{1a} is —SO₂NHMe, and R^{1b} is —CO₂Me; In some embodiments, R^{1a} is —SO₂NHMe, and R^{1b} is hydroxymethyl; In some embodiments, R^{1a} is —SO₂NHMe, and R^{1b} is hydroxyethyl; In some embodiments, R^{1a} is —SO₂NHMe, and R^{1b} is 2-hydroxy-2-propyl; In some embodiments, R^{1a} is —SO₂NHMe, and R^{1b} is —SO₂NHCH₂CH₂OH; In some embodiments, R^{1a} is —SO₂NHMe, and R^{1b} is —SO₂Me; In some embodiments, R^{1a} is —SO₂NHMe, and R^{1b} is CONHMe; In some embodiments, R^{1a} is —SO₂NHMe, and R^{1b} is dimethylaminomethyl; In some embodiments, R^{1a} is —SO₂NHMe, and R^{1b} is —CN; In some embodiments, R^{1a} is —SO₂NHMe, and R^{1b} is cyanomethyl; and In some embodiments, R^{1a} is C₁-C₄ alkyl substituted with one —OSi(Me)₂tBu, and R^{1b} is —CO₂Me.

In some embodiments of the compound of formula AA,

the substituted ring A is

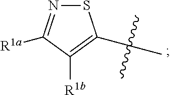

R^{1a} and R^{1b} are one of the following combinations:
R^{1a} is C₁-C₆ alkyl substituted with one or more hydroxy, and R^{1b} is C₁-C₆ alkyl substituted with one or more hydroxy; R^{1a} is C₁-C₆ alkyl substituted with one or more hydroxy, and R^{1b} is —SO₂NR¹¹R¹²; R^{1a} is C₁-C₆ alkyl substituted with one or more hydroxy, and R^{1b} is —SO₂R¹³; R^{1a} is C₁-C₆ alkyl substituted with one or more hydroxy, and R^{1b} is —CONR¹¹R¹²; R^{1a} is C₁-C₆ alkyl substituted with one or more hydroxy, and R^{1b} is —OR¹¹; R^{1a} is C₁-C₆ alkyl substituted with one or more hydroxy, and R^{1b} is —COR¹³; R^{1a} is C₁-C₆ alkyl substituted with one or more hydroxy, and R^{1b} is —CO₂R¹³; R^{1a} is C₁-C₆ alkyl substituted with one or more hydroxy, and R^{1b} is —NR¹³CONR¹¹R¹²; R^{1a} is C₁-C₆ alkyl substituted with one or more hydroxy, and R^{1b} is —CR¹¹R¹²CN; R^{1a} is C₁-C₆ alkyl substituted with one or more hydroxy, and R^{1b} is —NR¹¹SO₂R¹³; R^{1a} is C₁-C₆ alkyl substituted with one or more hydroxy, and R^{1b} is —NR¹¹CONR¹¹R¹²; R^{1a} is C₁-C₆ alkyl substituted with one or more hydroxy, and R^{1b} is —CN; R^{1a} is C₁-C₆ alkyl substituted with one or more hydroxy, and R^{1b} is —NR¹¹COR¹²; R^{1a} is C₁-C₆ alkyl substituted with one or more hydroxy, and R^{1b} is —CR¹¹R¹²NR¹¹R¹²; R^{1a} is C₁-C₆ alkyl substituted with one or more —OSi(R¹³)₃, and R^{1b} is C₁-C₆ alkyl substituted with one or more hydroxy; R^{1a} is C₁-C₆ alkyl substituted with one or more —OSi(R¹³)₃, and R^{1b} is —SO₂NR¹¹R¹²; R^{1a} is C₁-C₆ alkyl substituted with one or more —OSi(R¹³)₃, and R^{1b} is —SO₂R¹³; R^{1a} is C₁-C₆ alkyl substituted with one or more —OSi(R¹³)₃, and R^{1b} is —CONR¹¹R¹²; R^{1a} is C₁-C₆ alkyl substituted with one or more —OSi(R¹³)₃, and R^{1b} is —OR¹¹; R^{1a} is C₁-C₆ alkyl substituted with one or more —OSi(R¹³)₃, and R^{1b} is —COR¹³; R^{1a} is C₁-C₆ alkyl substituted with one or more —OSi(R¹³)₃, and R^{1b} is —CO₂R¹³; R^{1a} is C₁-C₆ alkyl substituted with one or more —OSi(R¹³)₃, and R^{1b} is —NR¹³CONR¹¹R¹²; R^{1a} is C₁-C₆ alkyl substituted with one or more —OSi(R¹³)₃, and R^{1b} is —CR¹¹R¹²CN; R^{1a} is C₁-C₆ alkyl substituted with one or more —OSi(R¹³)₃, and R^{1b} is —NR¹¹SO₂R¹³; R^{1a} is C₁-C₆ alkyl substituted with one or more —OSi(R¹³)₃, and R^{1b} is —NR¹¹CONR¹¹R¹²; R^{1a} is C₁-C₆ alkyl substituted with one or more —OSi(R¹³)₃, and R^{1b} is —CN; R^{1a} is C₁-C₆ alkyl substituted with one or more —OSi(R¹³)₃, and R^{1b} is —NR¹¹COR¹²; R^{1a} is C₁-C₆ alkyl substituted with one or more —OSi(R¹³)₃, and R^{1b} is —CR¹¹R¹²NR¹¹R¹²; R^{1a} is —SO₂NR¹¹R¹², and R^{1b} is C₁-C₆ alkyl substituted with one or more hydroxy; R^{1a} is —SO₂NR¹¹R¹², and R^{1b} is —SO₂NR¹¹R¹²; R^{1a} is —SO₂NR¹¹R¹², and R^{1b} is —SO₂R¹³; R^{1a} is —SO₂NR¹¹R¹², and R^{1b} is —CONR¹¹R¹²; R^{1a} is —SO₂NR¹¹R¹², and R^{1b} is —OR¹¹; R^{1a} is —SO₂NR¹¹R¹², and R^{1b} is —COR¹³; R^{1a} is —SO₂NR¹¹R¹², and R^{1b} is —CO₂R¹³; R^{1a} is —SO₂NR¹¹R¹², and R^{1b} is —NR¹³CONR¹¹R¹²; R^{1a} is —SO₂NR¹¹R¹², and R^{1b} is —CR¹¹R¹²CN; R^{1a} is —SO₂NR¹¹R¹², and R^{1b} is —NR¹¹SO₂R¹³; R^{1a} is —SO₂NR¹¹R¹², and R^{1b} is —CR¹¹R¹²NR¹¹R¹²; R^{1a} is —SO₂NR¹¹R¹², and R^{1b} is —CN; R^{1a} is —SO₂NR¹¹R¹², and R^{1b} is —NR¹¹CONR¹¹R¹²; R^{1a} is —SO₂NR¹¹R¹², and R^{1b} is —NR¹¹COR¹²; R^{1a} is —CR¹¹R¹²NR¹¹R¹², and R^{1b} is C₁-C₆ alkyl substituted with one or more hydroxy; R^{1a} is —CR¹¹R¹²NR¹¹R¹², and R^{1b} is —SO₂NR¹¹R¹²; R^{1a} is —CR¹¹R¹²NR¹¹R¹², and R^{1b} is —SO₂R¹³; R^{1a} is —CR¹¹R¹²NR¹¹R¹², and R^{1b} is —CONR¹¹R¹²; R^{1a} is —CR¹¹R¹²NR¹¹R¹², and R^{1b} is —OR¹¹; R^{1a} is —CR¹¹R¹²NR¹¹R¹², and R^{1b} is —COR¹³; R^{1a} is —CR¹¹R¹²NR¹¹R¹², and R^{1b} is —CO₂R¹³; R^{1a} is —CR¹¹R¹²NR¹¹R¹², and R^{1b} is —NR¹³CONR¹¹R¹²; R^{1a} is —CR¹¹R¹²NR¹¹R¹², and R^{1b} is —CR¹¹R¹²CN; R^{1a} is —CR¹¹R¹²NR¹¹R¹², and R^{1b} is —NR¹¹SO₂R¹³; R^{1a} is —CR¹¹R¹²NR¹¹R¹², and R^{1b} is —CR¹¹R¹²NR¹¹R¹²; R^{1a} is —CR¹¹R¹²NR¹¹R¹², and R^{1b} is —CN; R^{1a} is —CR¹¹R¹²NR¹¹R¹², and R^{1b} is —NR¹¹CONR¹¹R¹²; and R^{1a} is —CR¹¹R¹²NR¹¹R¹², and R^{1b} is —NR¹¹COR¹².

In some embodiments of the compound of formula AA,

the substituted ring A is

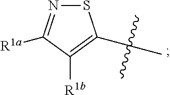

R^{1a} and R^{1b} are one of the following combinations:

In some embodiments, R^{1a} is 2-hydroxy-2-propyl, and R^{1b} is —OMe; In some embodiments, R^{1a} is 2-hydroxy-2-propyl, and R^{1b} is —OH; In some embodiments, R^{1a} is 2-hydroxy-2-propyl, and R^{1b} is —CO₂Me; In some embodiments, R^{1a} is 2-hydroxy-2-propyl, and R^{1b} is hydroxymethyl; In some embodiments, R^{1a} is 2-hydroxy-2-propyl, and R^{1b} is hydroxy ethyl; In some embodiments, R^{1a} is 2-hydroxy-2-propyl, and R^{1b} is 2-hydroxy-2-propyl; In some embodiments, R^{1a} is 2-hydroxy-2-propyl, and R^{1b} is —SO₂NHCH₂CH₂OH; In some embodiments, R^{1a} is 2-hydroxy-2-propyl, and R^{1b} is —SO₂Me; In some embodiments, R^{1a} is 2-hydroxy-2-propyl, and R^{1b} is CONHMe; In some embodiments, R^{1a} is 2-hydroxy-2-propyl, and R^{1b} is cyanomethyl; In some embodiments, R^{1a} is 2-hydroxy-2-propyl, and R^{1b} is —CN; In some embodiments, R^{1a} is 2-hydroxy-2-propyl, and R^{1b} is dimethylaminomethyl; In some embodiments, R^{1a} is dimethylaminomethyl, and R^{1b} is —OMe; In some embodiments, R^{1a} is dimethylaminomethyl, and R^{1b} is —OH; In some embodiments, R^{1a} is dimethylaminomethyl, and R^{1b} is —CO₂Me; In some embodiments, R^{1a} is dimethylaminomethyl, and R^{1b} is hydroxymethyl; In some embodiments, R^{1a} is dimethylaminomethyl, and R^{1b} is hydroxy ethyl; In some embodiments, R^{1a} is dimethylaminomethyl, and R^{1b} is 2-hydroxy-2-propyl; In some embodiments, R^{1a} is dimethylaminomethyl, and R^{1b} is —SO₂NHCH₂CH₂OH; In some embodiments, R^{1a} is dimethylaminomethyl, and R^{1b} is —SO₂Me; In some embodiments, R^{1a} is dimethylaminomethyl, and R^{1b} is CONHMe; In some embodiments, R^{1a} is dimethylaminomethyl, and R^{1b} is cyanomethyl; In some embodiments, R^{1a} is dimethylaminomethyl, and R^{1b} is dimethylaminomethyl; In some embodiments, R^{1a} is dimethylaminomethyl, and R^{1b} is CN. In some embodiments, R^{1a} is —SO₂NHMe, and R^{1b} is —OMe; In some embodiments, R^{1a} is —SO₂NHMe, and R^{1b} is —OH; In some embodiments, R^{1a} is —SO₂NHMe, and R^{1b} is —CO₂Me; In some embodiments, R^{1a} is —SO₂NHMe, and R^{1b} is hydroxymethyl; In some embodiments, R^{1a} is —SO₂NHMe, and R^{1b} is hydroxyethyl; In some embodiments, R^{1a} is —SO₂NHMe, and R^{1b} is 2-hydroxy-2-propyl; In some embodiments, R^{1a} is —SO₂NHMe, and R^{1b} is —SO₂NHCH₂CH₂OH; In some embodiments, R^{1a} is —SO₂NHMe, and R^{1b} is —SO₂Me; In some embodiments, R^{1a} is —SO₂NHMe, and R^{1b} is CONHMe; In some embodiments, R^{1a} is —SO₂NHMe, and R^{1b} is dimethylaminomethyl; In some embodiments, R^{1a} is —SO₂NHMe, and R^{1b} is —CN; In some embodiments, R^{1a} is —SO₂NHMe, and R^{1b} is cyanomethyl; and In some embodiments, R^{1a} is C₁-C₄ alkyl substituted with one —OSi(Me)₂tBu, and R^{1b} is —CO₂Me.

In some embodiments of the compound of formula AA,

the substituted ring A is

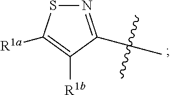

R^{1a} and R^{1b} are one of the following combinations:
R^{1a} is C₁-C₆ alkyl substituted with one or more hydroxy, and R^{1b} is C₁-C₆ alkyl substituted with one or more hydroxy; R^{1a} is C₁-C₆ alkyl substituted with one or more hydroxy, and R^{1b} is —SO₂NR¹¹R¹²; R^{1a} is C₁-C₆ alkyl substituted with one or more hydroxy, and R^{1b} is —SO₂R¹³; R^{1a} is C₁-C₆ alkyl substituted with one or more hydroxy, and R^{1b} is —CONR¹¹R¹²; R^{1a} is C₁-C₆ alkyl substituted with one or more hydroxy, and R^{1b} is —OR¹¹; R^{1a} is C₁-C₆ alkyl substituted with one or more hydroxy, and R^{1b} is —COR¹³; R^{1a} is C₁-C₆ alkyl substituted with one or more hydroxy, and R^{1b} is —CO₂R¹³; R^{1a} is C₁-C₆ alkyl substituted with one or more hydroxy, and R^{1b} is —NR¹³CONR¹¹R¹²; R^{1a} is C₁-C₆ alkyl substituted with one or more hydroxy, and R^{1b} is —CR¹¹R¹²CN; R^{1a} is C₁-C₆ alkyl substituted with one or more hydroxy, and R^{1b} is —NR¹¹SO₂R¹³; R^{1a} is C₁-C₆ alkyl substituted with one or more hydroxy, and R^{1b} is —NR¹¹CONR¹¹R¹²; R^{1a} is C₁-C₆ alkyl substituted with one or more hydroxy, and R^{1b} is —CN; R^{1a} is C₁-C₆ alkyl substituted with one or more hydroxy, and R^{1b} is —NR¹¹COR¹²; R^{1a} is C₁-C₆ alkyl substituted with one or more hydroxy, and R^{1b} is —CR¹¹R¹²NR¹¹R¹²; R^{1a} is C₁-C₆ alkyl substituted with one or more —OSi(R¹³)₃, and R^{1b} is C₁-C₆ alkyl substituted with one or more hydroxy; R^{1a} is C₁-C₆ alkyl substituted with one or more —OSi(R¹³)₃, and R^{1b} is —SO₂NR¹¹R¹²; R^{1a} is C₁-C₆ alkyl substituted with one or more —OSi(R¹³)₃, and R^{1b} is —SO₂R¹³; R^{1a} is C₁-C₆ alkyl substituted with one or more —OSi(R¹³)₃, and R^{1b} is —CONR¹¹R¹²; R^{1a} is C₁-C₆ alkyl substituted with one or more —OSi(R¹³)₃, and R^{1b} is —OR¹¹; R^{1a} is C₁-C₆ alkyl substituted with one or more —OSi(R¹³)₃, and R^{1b} is —COR¹³; R^{1a} is C₁-C₆ alkyl substituted with one or more —OSi(R¹³)₃, and R^{1b} is —CO₂R¹³; R^{1a} is C₁-C₆ alkyl substituted with one or more —OSi(R¹³)₃, and R^{1b} is —NR¹³CONR¹¹R¹²; R^{1a} is C₁-C₆ alkyl substituted with one or more —OSi(R¹³)₃, and R^{1b} is —CR¹¹R¹²CN; R^{1a} is C₁-C₆ alkyl substituted with one or more —OSi(R¹³)₃, and R^{1b} is —NR¹¹SO₂R¹³; R^{1a} is C₁-C₆ alkyl substituted with one or more —OSi(R¹³)₃, and R^{1b} is —NR¹¹CONR¹¹R¹²; R^{1a} is C₁-C₆ alkyl substituted with one or more —OSi(R¹³)₃, and R^{1b} is —CN; R^{1a} is C₁-C₆ alkyl substituted with one or more —OSi(R¹³)₃, and R^{1b} is —NR¹¹COR¹²; R^{1a} is C₁-C₆ alkyl substituted with one or more —OSi(R¹³)₃, and R^{1b} is —CR¹¹R¹²NR¹¹R¹²; R^{1a} is —SO₂NR¹¹R¹², and R^{1b} is C₁-C₆ alkyl substituted with one or more hydroxy; R^{1a} is —SO₂NR¹¹R¹², and R^{1b} is —SO₂NR¹¹R¹²; R^{1a} is —SO₂NR¹¹R¹², and R^{1b} is —SO₂R¹³; R^{1a} is —SO₂NR¹¹R¹², and R^{1b} is —CONR¹¹R¹²; R^{1a} is —SO₂NR¹¹R¹², and R^{1b} is —OR¹¹; R^{1a} is —SO₂NR¹¹R¹², and R^{1b} is —COR¹³; R^{1a} is —SO₂NR¹¹R¹², and R^{1b} is —CO₂R¹³; R^{1a} is —SO₂NR¹¹R¹², and R^{1b} is —NR¹³CONR¹¹R¹²; R^{1a} is —SO₂NR¹¹R¹², and R^{1b} is —CR¹¹R¹²CN; R^{1a} is —SO₂NR¹¹R¹², and R^{1b} is —NR¹¹SO₂R¹³; R^{1a} is —SO₂NR¹¹R¹², and R^{1b} is —CR¹¹R¹²NR¹¹R¹²; R^{1a} is —SO₂NR¹¹R¹², and R^{1b} is —CN; R^{1a} is —SO₂NR¹¹R¹², and R^{1b} is —NR¹¹CONR¹¹R¹²; R^{1a} is —SO₂NR¹¹R¹², and R^{1b} is —NR¹¹COR¹²; R^{1a} is —CR¹¹R¹²NR¹¹R¹², and R^{1b} is C₁-C₆ alkyl substituted with one or more hydroxy; R^{1a} is —CR¹¹R¹²NR¹¹R¹², and R^{1b} is —SO₂NR¹¹R¹²; R^{1a} is —CR¹¹R¹²NR¹¹R¹², and R^{1b} is —SO₂R¹³; R^{1a} is —CR¹¹R¹²NR¹¹R¹², and R^{1b} is —CONR¹¹R¹²; R^{1a} is —CR¹¹R¹²NR¹¹R¹², and R^{1b} is —OR¹¹; R^{1a} is —CR¹¹R¹²NR¹¹R¹², and R^{1b} is —COR¹³; R^{1a} is —CR¹¹R¹²NR¹¹R¹², and R^{1b} is —CO₂R¹³; R^{1a} is —CR¹¹R¹²NR¹¹R¹², and R^{1b} is —NR¹³CONR¹¹R¹²; R^{1a} is —CR¹¹R¹²NR¹¹R¹², and R^{1b} is —CR¹¹R¹²CN; R^{1a} is —CR¹¹R¹²NR¹¹R¹², and R^{1b} is —NR¹¹SO₂R¹³; R^{1a} is —CR¹¹R¹²NR¹¹R¹², and R^{1b} is —CR¹¹R¹²NR¹¹R¹²; R^{1a} is —CR¹¹R¹²NR¹¹R¹², and R^{1b} is —CN; R^{1a} is —CR¹¹R¹²NR¹¹R¹², and R^{1b} is —NR¹¹CONR¹¹R¹²; and R^{1a} is —CR¹¹R¹²NR¹¹R¹², and R^{1b} is —NR¹¹COR¹².

In some embodiments of the compound of formula AA,

the substituted ring A is

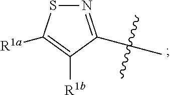

R^{1a} and R^{1b} are one of the following combinations:

In some embodiments, R^{1a} is 2-hydroxy-2-propyl, and R^{1b} is —OMe; In some embodiments, R^{1a} is 2-hydroxy-2-propyl, and R^{1b} is —OH; In some embodiments, R^{1a} is 2-hydroxy-2-propyl, and R^{1b} is —CO₂Me; In some embodiments, R^{1a} is 2-hydroxy-2-propyl, and R^{1b} is hydroxymethyl; In some embodiments, R^{1a} is 2-hydroxy-2-propyl, and R^{1b} is hydroxy ethyl; In some embodiments, R^{1a} is 2-hydroxy-2-propyl, and R^{1b} is 2-hydroxy-2-propyl; In some embodiments, R^{1a} is 2-hydroxy-2-propyl, and R^{1b} is —SO₂NHCH₂CH₂OH; In some embodiments, R^{1a} is 2-hydroxy-2-propyl, and R^{1b} is —SO₂Me; In some embodiments, R^{1a} is 2-hydroxy-2-propyl, and R^{1b} is CONHMe; In some embodiments, R^{1a} is 2-hydroxy-2-propyl, and R^{1b} is cyanomethyl; In some embodiments, R^{1a} is 2-hydroxy-2-propyl, and R^{1b} is —CN; In some embodiments, R^{1a} is 2-hydroxy-2-propyl, and R^{1b} is dimethylaminomethyl; In some embodiments, R^{1a} is dimethylaminomethyl, and R^{1b} is —OMe; In some embodiments, R^{1a} is dimethylaminomethyl, and R^{1b} is —OH; In some embodiments, R^{1a} is dimethylaminomethyl, and R^{1b} is —CO₂Me; In some embodiments, R^{1a} is dimethylaminomethyl, and R^{1b} is hydroxymethyl; In some embodiments, R^{1a} is dimethylaminomethyl, and R^{1b} is hydroxy ethyl; In some embodiments, R^{1a} is dimethylaminomethyl, and R^{1b} is 2-hydroxy-2-propyl; In some embodiments, R^{1a} is dimethylaminomethyl, and R^{1b} is —SO₂NHCH₂CH₂OH; In some embodiments, R^{1a} is dimethylaminomethyl, and R^{1b} is —SO₂Me; In some embodiments, R^{1a} is dimethylaminomethyl, and R^{1b} is CONHMe; In some embodiments, R^{1a} is dimethylaminomethyl, and R^{1b} is cyanomethyl; In some embodiments, R^{1a} is dimethylaminomethyl, and R^{1b} is dimethylaminomethyl; In some embodiments, R^{1a} is dimethylaminomethyl, and R^{1b} is CN. In some embodiments, R^{1a} is —SO₂NHMe, and R^{1b} is —OMe; In some embodiments, R^{1a} is —SO₂NHMe, and R^{1b} is —OH; In some embodiments, R^{1a} is —SO₂NHMe, and R^{1b} is —CO₂Me; In some embodiments, R^{1a} is —SO₂NHMe, and R^{1b} is hydroxymethyl; In some embodiments, R^{1a} is —SO₂NHMe, and R^{1b} is hydroxyethyl; In some embodiments, R^{1a} is —SO₂NHMe, and R^{1b} is 2-hydroxy-2-propyl; In some embodiments, R^{1a} is —SO₂NHMe, and R^{1b} is —SO₂NHCH₂CH₂OH; In some embodiments, R^{1a} is —SO₂NHMe, and R^{1b} is —SO₂Me; In some embodiments, R^{1a} is —SO₂NHMe, and R^{1b} is CONHMe; In some embodiments, R^{1a} is —SO₂NHMe, and R^{1b} is dimethylaminomethyl; In some embodiments, R^{1a} is —SO₂NHMe, and R^{1b} is —CN; In some embodiments, R^{1a} is —SO₂NHMe, and R^{1b} is cyanomethyl; and In some embodiments, R^{1a} is C₁-C₄ alkyl substituted with one —OSi(Me)₂tBu, and R^{1b} is —CO₂Me.

In some embodiments of the compound of formula AA,

the substituted ring A is

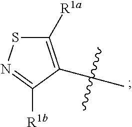

R^{1a} and R^{1b} are one of the following combinations:
R^{1a} is C₁-C₆ alkyl substituted with one or more hydroxy, and R^{1b} is C₁-C₆ alkyl substituted with one or more hydroxy; R^{1a} is C₁-C₆ alkyl substituted with one or more hydroxy, and R^{1b} is —SO₂NR¹¹R¹²; R^{1a} is C₁-C₆ alkyl substituted with one or more hydroxy, and R^{1b} is —SO₂R¹³; R^{1a} is C₁-C₆ alkyl substituted with one or more hydroxy, and R^{1b} is —CONR¹¹R¹²; R^{1a} is C₁-C₆ alkyl substituted with one or more hydroxy, and R^{1b} is —OR¹¹; R^{1a} is C₁-C₆ alkyl substituted with one or more hydroxy, and R^{1b} is —COR¹³; R^{1a} is C₁-C₆ alkyl substituted with one or more hydroxy, and R^{1b} is —CO₂R¹³; R^{1a} is C₁-C₆ alkyl substituted with one or more hydroxy, and R^{1b} is —NR¹³CONR¹¹R¹²; R^{1a} is C₁-C₆ alkyl substituted with one or more hydroxy, and R^{1b} is —CR¹¹R¹²CN; R^{1a} is C₁-C₆ alkyl substituted with one or more hydroxy, and R^{1b} is —NR¹¹SO₂R¹³; R^{1a} is C₁-C₆ alkyl substituted with one or more hydroxy, and R^{1b} is —NR¹¹CONR¹¹R¹²; R^{1a} is C₁-C₆ alkyl substituted with one or more hydroxy, and R^{1b} is —CN; R^{1a} is C₁-C₆ alkyl substituted with one or more hydroxy, and R^{1b} is —NR¹¹COR¹²; R^{1a} is C₁-C₆ alkyl substituted with one or more hydroxy, and R^{1b} is —CR¹¹R¹²NR¹¹R¹²; R^{1a} is C₁-C₆ alkyl substituted with one or more —OSi(R¹³)₃, and R^{1b} is C₁-C₆ alkyl substituted with one or more hydroxy; R^{1a} is C₁-C₆ alkyl substituted with one or more —OSi(R¹³)₃, and R^{1b} is —SO₂NR¹¹R¹²; R^{1a} is C₁-C₆ alkyl substituted with one or more —OSi(R¹³)₃, and R^{1b} is —SO₂R¹³; R^{1a} is C₁-C₆ alkyl substituted with one or more —OSi(R¹³)₃, and R^{1b} is —CONR¹¹R¹²; R^{1a} is C₁-C₆ alkyl substituted with one or more —OSi(R¹³)₃, and R^{1b} is —OR¹¹; R^{1a} is C₁-C₆ alkyl substituted with one or more —OSi(R¹³)₃, and R^{1b} is —COR¹³; R^{1a} is C₁-C₆ alkyl substituted with one or more —OSi(R¹³)₃, and R^{1b} is —CO₂R¹³; R^{1a} is C₁-C₆ alkyl substituted with one or more —OSi(R¹³)₃, and R^{1b} is —NR¹³CONR¹¹R¹²; R^{1a} is C₁-C₆ alkyl substituted with one or more —OSi(R¹³)₃, and R^{1b} is —CR¹¹R¹²CN; R^{1a} is C₁-C₆ alkyl substituted with one or more —OSi(R¹³)₃, and R^{1b} is —NR¹¹SO₂R¹³; R^{1a} is C₁-C₆ alkyl substituted with one or more —OSi(R¹³)₃, and R^{1b} is —NR¹¹CONR¹¹R¹²; R^{1a} is C₁-C₆ alkyl substituted with one or more —OSi(R¹³)₃, and R^{1b} is —CN; R^{1a} is C₁-C₆ alkyl substituted with one or more —OSi(R¹³)₃, and R^{1b} is —NR¹¹COR¹²; R^{1a} is C₁-C₆ alkyl substituted with one or more —OSi(R¹³)₃, and R^{1b} is —CR¹¹R¹²NR¹¹R¹²; R^{1a} is —SO₂NR¹¹R¹², and R^{1b} is C₁-C₆ alkyl substituted with one or more hydroxy; R^{1a} is —SO₂NR¹¹R¹², and R^{1b} is —SO₂NR¹¹R¹²; R^{1a} is —SO₂NR¹¹R¹², and R^{1b} is —SO₂R¹³; R^{1a} is —SO₂NR¹¹R¹², and R^{1b} is —CONR¹¹R¹²; R^{1a} is —SO₂NR¹¹R¹², and R^{1b} is —OR¹¹; R^{1a} is —SO₂NR¹¹R¹², and R^{1b} is —COR¹³; R^{1a} is —SO₂NR¹¹R¹², and R^{1b} is —CO₂R¹³; R^{1a} is —SO₂NR¹¹R¹², and R^{1b} is —NR¹³CONR¹¹R¹²; R^{1a} is —SO₂NR¹¹R¹², and R^{1b} is —CR¹¹R¹²CN; R^{1a} is —SO₂NR¹¹R¹², and R^{1b} is —NR¹¹SO₂R¹³; R^{1a} is —SO₂NR¹¹R¹², and R^{1b} is —CR¹¹R¹²NR¹¹R¹²; R^{1a} is —SO₂NR¹¹R¹², and R^{1b} is —CN; R^{1a} is —SO₂NR¹¹R¹², and R^{1b} is —NR¹¹CONR¹¹R¹²; R^{1a} is —SO₂NR¹¹R¹², and R^{1b} is —NR¹¹COR¹²; R^{1a} is —CR¹¹R¹²NR¹¹R¹², and R^{1b} is C₁-C₆ alkyl substituted with one or more hydroxy; R^{1a} is —CR¹¹R¹²NR¹¹R¹², and R^{1b} is —SO₂NR¹¹R¹²; R^{1a} is —CR¹¹R¹²NR¹¹R¹², and R^{1b} is —SO₂R¹³; R^{1a} is —CR¹¹R¹²NR¹¹R¹², and R^{1b} is —CONR¹¹R¹²; R^{1a} is —CR¹¹R¹²NR¹¹R¹², and R^{1b} is —OR¹¹; R^{1a} is —CR¹¹R¹²NR¹¹R¹², and R^{1b} is —COR¹³; R^{1a} is —CR¹¹R¹²NR¹¹R¹², and R^{1b} is —CO₂R¹³; R^{1a} is —CR¹¹R¹²NR¹¹R¹², and R^{1b} is —NR¹³CONR¹¹R¹²; R^{1a} is —CR¹¹R¹²NR¹¹R¹², and R^{1b} is —CR¹¹R¹²CN; R^{1a} is —CR¹¹R¹²NR¹¹R¹², and R^{1b} is —NR¹¹SO₂R¹³; R^{1a} is —CR¹¹R¹²NR¹¹R¹², and R^{1b} is —CR¹¹R¹²NR¹¹R¹²; R^{1a} is —CR¹¹R¹²NR¹¹R¹², and R^{1b} is —CN; R^{1a} is —CR¹¹R¹²NR¹¹R¹², and R^{1b} is —NR¹¹CONR¹¹R¹²; and R^{1a} is —CR¹¹R¹²NR¹¹R¹², and R^{1b} is —NR¹¹COR¹².

In some embodiments of the compound of formula AA,

the substituted ring A is

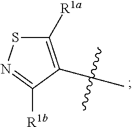

R^{1a} and R^{1b} are one of the following combinations:

In some embodiments, R^{1a} is 2-hydroxy-2-propyl, and R^{1b} is —OMe; In some embodiments, R^{1a} is 2-hydroxy-2-propyl, and R^{1b} is —OH; In some embodiments, R^{1a} is 2-hydroxy-2-propyl, and R^{1b} is —CO₂Me; some embodiments, R^{1a} is 2-hydroxy-2-propyl, and R^{1b} is hydroxymethyl; In some embodiments, R^{1a} is 2-hydroxy-2-propyl, and R^{1b} is hydroxyethyl; some embodiments, R^{1a} is 2-hydroxy-2-propyl, and R^{1b} is 2-hydroxy-2-propyl; In some embodiments, R^{1a} is 2-hydroxy-2-propyl, and R^{1b} is —SO₂NHCH₂CH₂OH; In some embodiments, R^{1a} is 2-hydroxy-2-propyl, and R^{1b} is —SO₂Me; In some embodiments, R^{1a} is 2-hydroxy-2-propyl, and R^{1b} is CONHMe; In some embodiments, R^{1a} is 2-hydroxy-2-propyl, and R^{1b} is cyanomethyl; In some embodiments, R^{1a} is 2-hydroxy-2-propyl, and R^{1b} is —CN; In some embodiments, R^{1a} is 2-hydroxy-2-propyl, and R^{1b} is dimethylaminomethyl; In some embodiments, R^{1a} is dimethylaminomethyl, and R^{1b} is —OMe; In some embodiments, R^{1a} is dimethylaminomethyl, and R^{1b} is —OH; In some embodiments, R^{1a} is dimethylaminomethyl, and R^{1b} is —CO₂Me; In some embodiments, R^{1a} is dimethylaminomethyl, and R^{1b} is hydroxymethyl; In some embodiments, R^{1a} is dimethylaminomethyl, and R^{1b} is hydroxyethyl; In some embodiments, R^{1a} is dimethylaminomethyl, and R^{1b} is 2-hydroxy-2-propyl; In some embodiments, R^{1a} is dimethylaminomethyl, and R^{1b} is —SO₂NHCH₂CH₂OH; In some embodiments, R^{1a} is dimethylaminomethyl, and R^{1b} is —SO₂Me; In some embodiments, R^{1a} is dimethylaminomethyl, and R^{1b} is CONHMe; In some embodiments, R^{1a} is dimethylaminomethyl, and R^{1b} is cyanomethyl; In some embodiments, R^{1a} is dimethylaminomethyl, and R^{1b} is dimethylaminomethyl; In some embodiments, R^{1a} is dimethylaminomethyl, and R^{1b} is CN. In some embodiments, R^{1a} is —SO₂NHMe, and R^{1b} is —OMe; In some embodiments, R^{1a} is —SO₂NHMe, and R^{1b} is —OH; In some embodiments, R^{1a} is —SO₂NHMe, and R^{1b} is —CO₂Me; In some embodiments, R^{1a} is —SO₂NHMe, and R^{1b} is hydroxymethyl; In some embodiments, R^{1a} is —SO₂NHMe, and R^{1b} is hydroxyethyl; In some embodiments, R^{1a} is —SO₂NHMe, and R^{1b} is 2-hydroxy-2-propyl; In some embodiments, R^{1a} is —SO₂NHMe, and R^{1b} is —SO₂NHCH₂CH₂OH; In some embodiments, R^{1a} is —SO₂NHMe, and R^{1b} is —SO₂Me; In some embodiments, R^{1a} is —SO₂NHMe, and R^{1b} is CONHMe; In some embodiments, R^{1a} is —SO₂NHMe, and R^{1b} is cyanomethyl; In some embodiments, R^{1a} is —SO₂NHMe, and R^{1b} is dimethylaminomethyl; In some embodiments, R^{1a} is —SO₂NHMe, and R^{1b} is —CN; and In some embodiments, R^{1a} is C₁-C₄ alkyl substituted with one —OSi(Me)₂tBu, and R^{1b} is —CO₂Me.

In some embodiments of the compound of formula AA,

the substituted ring A is

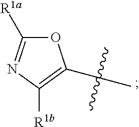

R^{1a} and R^{1b} are one of the following combinations:

In some embodiments, R^{1a} is 2-hydroxy-2-propyl, and R^{1b} is —OMe; In some embodiments, R^{1a} is 2-hydroxy-2-propyl, and R^{1b} is —OH; In some embodiments, R^{1a} is 2-hydroxy-2-propyl, and R^{1b} is —CO₂Me; In some embodiments, R^{1a} is 2-hydroxy-2-propyl, and R^{1b} is hydroxymethyl; In some embodiments, R^{1a} is 2-hydroxy-2-propyl, and R^{1b} is hydroxyethyl; In some embodiments, R^{1a} is 2-hydroxy-2-propyl, and R^{1b} is 2-hydroxy-2-propyl; In some embodiments, R^{1a} is 2-hydroxy-2-propyl, and R^{1b} is —SO₂NHCH₂CH₂OH; In some embodiments, R^{1a} is 2-hydroxy-2-propyl, and R^{1b} is —SO₂Me; In some embodiments, R^{1a} is 2-hydroxy-2-propyl, and R^{1b} is CONHMe; In some embodiments, R^{1a} is 2-hydroxy-2-propyl, and R^{1b} is cyanomethyl; In some embodiments, R^{1a} is 2-hydroxy-2-propyl, and R^{1b} is —CN; In some embodiments, R^{1a} is 2-hydroxy-2-propyl, and R^{1b} is dimethylaminomethyl; In some embodiments, R^{1a} is dimethylaminomethyl, and R^{1b} is —OMe; In some embodiments, R^{1a} is dimethylaminomethyl, and R^{1b} is —OH; In some embodiments, R^{1a} is dimethylaminomethyl, and R^{1b} is —CO₂Me; In some embodiments, R^{1a} is dimethylaminomethyl, and R^{1b} is hydroxymethyl; In some embodiments, R^{1a} is dimethylaminomethyl, and R^{1b} is hydroxy ethyl; In some embodiments, R^{1a} is dimethylaminomethyl, and R^{1b} is 2-hydroxy-2-propyl; In some embodiments, R^{1a} is dimethylaminomethyl, and R^{1b} is —SO₂NHCH₂CH₂OH; In some embodiments, R^{1a} is dimethylaminomethyl, and R^{1b} is —SO₂Me; In some embodiments, R^{1a} is dimethylaminomethyl, and R^{1b} is CONHMe; In some embodiments, R^{1a} is dimethylaminomethyl, and R^{1b} is cyanomethyl; In some embodiments, R^{1a} is dimethylaminomethyl, and R^{1b} is dimethylaminomethyl; In some embodiments, R^{1a} is dimethylaminomethyl, and R^{1b} is CN. In some embodiments, R^{1a} is —SO₂NHMe, and R^{1b} is —OMe; In some embodiments, R^{1a} is —SO₂NHMe, and R^{1b} is —OH; In some embodiments, R^{1a} is —SO₂NHMe, and R^{1b} is —CO₂Me; In some embodiments, R^{1a} is —SO₂NHMe, and R^{1b} is hydroxymethyl; In some embodiments, R^{1a} is —SO₂NHMe, and R^{1b} is hydroxyethyl; In some embodiments, R^{1a} is —SO₂NHMe, and R^{1b} is 2-hydroxy-2-propyl; In some embodiments, R^{1a} is —SO₂NHMe, and R^{1b} is —SO₂NHCH₂CH₂OH; In some embodiments, R^{1a} is —SO₂NHMe, and R^{1b} is —SO₂Me; In some embodiments, R^{1a} is —SO₂NHMe, and R^{1b} is CONHMe; In some embodiments, R^{1a} is —SO₂NHMe, and R^{1b} is dimethylaminomethyl; In some embodiments, R^{1a} is —SO₂NHMe, and R^{1b} is —CN; In some embodiments, R^{1a} is —SO₂NHMe, and R^{1b} is cyanomethyl; and In some embodiments, R^{1a} is C₁-C₄ alkyl substituted with one —OSi(Me)₂tBu, and R^{1b} is —CO₂Me.

In some embodiments of the compound of formula AA,

the substituted ring A is

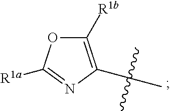

R^{1a} and R^{1b} are one of the following combinations:
R^{1a} is C₁-C₆ alkyl substituted with one or more hydroxy, and R^{1b} is C₁-C₆ alkyl substituted with one or more hydroxy; R^{1a} is C₁-C₆ alkyl substituted with one or more hydroxy, and R^{1b} is —SO₂NR¹¹R¹²; R^{1a} is C₁-C₆ alkyl substituted with one or more hydroxy, and R^{1b} is —SO₂R¹³; R^{1a} is C₁-C₆ alkyl substituted with one or more hydroxy, and R^{1b} is —CONR¹¹R¹²; R^{1a} is C₁-C₆ alkyl substituted with one or more hydroxy, and R^{1b} is —OR¹¹; R^{1a} is C₁-C₆ alkyl substituted with one or more hydroxy, and R^{1b} is —COR¹³; R^{1a} is C₁-C₆ alkyl substituted with one or more hydroxy, and R^{1b} is —CO₂R¹³; R^{1a} is C₁-C₆ alkyl substituted with one or more hydroxy, and R^{1b} is —NR¹³CONR¹¹R¹²; R^{1a} is C₁-C₆ alkyl substituted with one or more hydroxy, and R^{1b} is —CR¹¹R¹²CN; R^{1a} is C₁-C₆ alkyl substituted with one or more hydroxy, and R^{1b} is —NR¹¹SO₂R¹³; R^{1a} is C₁-C₆ alkyl substituted with one or more hydroxy, and R^{1b} is —NR¹¹CONR¹¹R¹²; R^{1a} is C₁-C₆ alkyl substituted with one or more hydroxy, and R^{1b} is —CN; R^{1a} is C₁-C₆ alkyl substituted with one or more hydroxy, and R^{1b} is —NR¹¹COR¹²; R^{1a} is C₁-C₆ alkyl substituted with one or more hydroxy, and R^{1b} is —CR¹¹R¹²NR¹¹R¹²; R^{1a} is C₁-C₆ alkyl substituted with one or more —OSi(R¹³)₃, and R^{1b} is C₁-C₆ alkyl substituted with one or more hydroxy; R^{1a} is C₁-C₆ alkyl substituted with one or more —OSi(R¹³)₃, and R^{1b} is —SO₂NR¹¹R¹²; R^{1a} is C₁-C₆ alkyl substituted with one or more —OSi(R¹³)₃, and R^{1b} is —SO₂R¹³; R^{1a} is C₁-C₆ alkyl substituted with one or more —OSi(R¹³)₃, and R^{1b} is —CONR¹¹R¹²; R^{1a} is C₁-C₆ alkyl substituted with one or more —OSi(R¹³)₃, and R^{1b} is —OR¹¹; R^{1a} is C₁-C₆ alkyl substituted with one or more —OSi(R¹³)₃, and R^{1b} is —COR¹³; R^{1a} is C₁-C₆ alkyl substituted with one or more —OSi(R¹³)₃, and R^{1b} is —CO₂R¹³; R^{1a} is C₁-C₆ alkyl substituted with one or more —OSi(R¹³)₃, and R^{1b} is —NR¹³CONR¹¹R¹²; R^{1a} is C₁-C₆ alkyl substituted with one or more —OSi(R¹³)₃, and R^{1b} is —CR¹¹R¹²CN; R^{1a} is C₁-C₆ alkyl substituted with one or more —OSi(R¹³)₃, and R^{1b} is —NR¹¹SO₂R¹³; R^{1a} is C₁-C₆ alkyl substituted with one or more —OSi(R¹³)₃, and R^{1b} is —NR¹¹CONR¹¹R¹²; R^{1a} is C₁-C₆ alkyl substituted with one or more —OSi(R¹³)₃, and R^{1b} is —CN; R^{1a} is C₁-C₆ alkyl substituted with one or more —OSi(R¹³)₃, and R^{1b} is —NR¹¹COR¹²; R^{1a} is C₁-C₆ alkyl substituted with one or more —OSi(R¹³)₃, and R^{1b} is —CR¹¹R¹²NR¹¹R¹²; R^{1a} is —SO₂NR¹¹R¹², and R^{1b} is C₁-C₆ alkyl substituted with one or more hydroxy; R^{1a} is —SO₂NR¹¹R¹², and R^{1b} is —SO₂NR¹¹R¹²; R^{1a} is —SO₂NR¹¹R¹², and R^{1b} is —SO₂R¹³; R^{1a} is —SO₂NR¹¹R¹², and R^{1b} is —CONR¹¹R¹²; R^{1a} is —SO₂NR¹¹R¹², and R^{1b} is —OR¹¹; R^{1a} is —SO₂NR¹¹R¹², and R^{1b} is —COR¹³; R^{1a} is —SO₂NR¹¹R¹², and R^{1b} is —CO₂R¹³; R^{1a} is —SO₂NR¹¹R¹², and R^{1b} is —NR¹³CONR¹¹R¹²; R^{1a} is —SO₂NR¹¹R¹², and R^{1b} is —CR¹¹R¹²CN; R^{1a} is —SO₂NR¹¹R¹², and R^{1b} is —NR¹¹SO₂R¹³; R^{1a} is —SO₂NR¹¹R¹², and R^{1b} is —CR¹¹R¹²NR¹¹R¹²; R^{1a} is —SO₂NR¹¹R¹², and R^{1b} is —CN; R^{1a} is —SO₂NR¹¹R¹², and R^{1b} is —NR¹¹CONR¹¹R¹²; R^{1a} is —SO₂NR¹¹R¹², and R^{1b} is —NR¹¹COR¹²; R^{1a} is —CR¹¹R¹²NR¹¹R¹², and R^{1b} is C₁-C₆ alkyl substituted with one or more hydroxy; R^{1a} is —CR¹¹R¹²NR¹¹R¹², and R^{1b} is —SO₂NR¹¹R¹²; R^{1a} is —CR¹¹R¹²NR¹¹R¹², and R^{1b} is —SO₂R¹³; R^{1a} is —CR¹¹R¹²NR¹¹R¹², and R^{1b} is —CONR¹¹R¹²; R^{1a} is —CR¹¹R¹²NR¹¹R¹², and R^{1b} is —OR¹¹; R^{1a} is —CR¹¹R¹²NR¹¹R¹², and R^{1b} is —COR¹³; R^{1a} is —CR¹¹R¹²NR¹¹R¹², and R^{1b} is —CO₂R¹³; R^{1a} is —CR¹¹R¹²NR¹¹R¹², and R^{1b} is —NR¹³CONR¹¹R¹²; R^{1a} is —CR¹¹R¹²NR¹¹R¹², and R^{1b} is —CR¹¹R¹²CN; R^{1a} is —CR¹¹R¹²NR¹¹R¹², and R^{1b} is —NR¹¹SO₂R¹³; R^{1a} is —CR¹¹R¹²NR¹¹R¹², and R^{1b} is —CR¹¹R¹²NR¹¹R¹²; R^{1a} is —CR¹¹R¹²NR¹¹R¹², and R^{1b} is —CN; R^{1a} is —CR¹¹R¹²NR¹¹R¹², and R^{1b} is —NR¹¹CONR¹¹R¹²; and R^{1a} is —CR¹¹R¹²NR¹¹R¹², and R^{1b} is —NR¹¹COR¹².

In some embodiments of the compound of formula AA,

the substituted ring A is

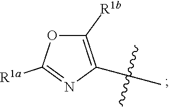

R^{1a} and R^{1b} are one of the following combinations:

In some embodiments, R^{1a} is 2-hydroxy-2-propyl, and R^{1b} is —OMe; In some embodiments, R^{1a} is 2-hydroxy-2-propyl, and R^{1b} is —OH; In some embodiments, R^{1a} is 2-hydroxy-2-propyl, and R^{1b} is —CO₂Me; In some embodiments, R^{1a} is 2-hydroxy-2-propyl, and R^{1b} is hydroxymethyl; In some embodiments, R^{1a} is 2-hydroxy-2-propyl, and R^{1b} is hydroxy ethyl; In some embodiments, R^{1a} is 2-hydroxy-2-propyl, and R^{1b} is 2-hydroxy-2-propyl; In some embodiments, R^{1a} is 2-hydroxy-2-propyl, and R^{1b} is —SO₂NHCH₂CH₂OH; In some embodiments, R^{1a} is 2-hydroxy-2-propyl, and R^{1b} is —SO₂Me; In some embodiments, R^{1a} is 2-hydroxy-2-propyl, and R^{1b} is CONHMe; In some embodiments, R^{1a} is 2-hydroxy-2-propyl, and R^{1b} is cyanomethyl; In some embodiments, R^{1a} is 2-hydroxy-2-propyl, and R^{1b} is —CN; In some embodiments, R^{1a} is 2-hydroxy-2-propyl, and R^{1b} is dimethylaminomethyl; In some embodiments, R^{1a} is dimethylaminomethyl, and R^{1b} is —OMe; In some embodiments, R^{1a} is dimethylaminomethyl, and R^{1b} is —OH; In some embodiments, R^{1a} is dimethylaminomethyl, and R^{1b} is —CO₂Me; In some embodiments, R^{1a} is dimethylaminomethyl, and R^{1b} is hydroxymethyl; In some embodiments, R^{1a} is dimethylaminomethyl, and R^{1b} is hydroxy ethyl; In some embodiments, R^{1a} is dimethylaminomethyl, and R^{1b} is 2-hydroxy-2-propyl; In some embodiments, R^{1a} is dimethylaminomethyl, and R^{1b} is —SO₂NHCH₂CH₂OH; In some embodiments, R^{1a} is dimethylaminomethyl, and R^{1b} is —SO₂Me; In some embodiments, R^{1a} is dimethylaminomethyl, and R^{1b} is CONHMe; In some embodiments, R^{1a} is dimethylaminomethyl, and R^{1b} is cyanomethyl; In some embodiments, R^{1a} is dimethylaminomethyl, and R^{1b} is dimethylaminomethyl; In some embodiments, R^{1a} is dimethylaminomethyl, and R^{1b} is CN. In some embodiments, R^{1a} is —SO₂NHMe, and R^{1b} is —OMe; In some embodiments, R^{1a} is —SO₂NHMe, and R^{1b} is —OH; In some embodiments, R^{1a} is —SO₂NHMe, and R^{1b} is —CO₂Me; In some embodiments, R^{1a} is —SO₂NHMe, and R^{1b} is hydroxymethyl; In some embodiments, R^{1a} is —SO₂NHMe, and R^{1b} is hydroxyethyl; In some embodiments, R^{1a} is —SO₂NHMe, and R^{1b} is 2-hydroxy-2-propyl; In some embodiments, R^{1a} is —SO₂NHMe, and R^{1b} is —SO₂NHCH₂CH₂OH; In some embodiments, R^{1a} is —SO₂NHMe, and R^{1b} is —SO₂Me; In some embodiments, R^{1a} is —SO₂NHMe, and R^{1b} is CONHMe; In some embodiments, R^{1a} is —SO₂NHMe, and R^{1b} is dimethylaminomethyl; In some embodiments, R^{1a} is —SO₂NHMe, and R^{1b} is —CN; In some embodiments, R^{1a} is —SO₂NHMe, and R^{1b} is cyanomethyl; and In some embodiments, R^{1a} is C₁-C₄ alkyl substituted with one —OSi(Me)₂tBu, and R^{1b} is —CO₂Me.

In some embodiments of the compound of formula AA,

the substituted ring A is

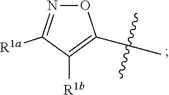

R^{1a} and R^{1b} are one of the following combinations:
R^{1a} is C₁-C₆ alkyl substituted with one or more hydroxy, and R^{1b} is C₁-C₆ alkyl substituted with one or more hydroxy; R^{1a} is C₁-C₆ alkyl substituted with one or more hydroxy, and R^{1b} is —SO₂NR¹¹R¹²; R^{1a} is C₁-C₆ alkyl substituted with one or more hydroxy, and R^{1b} is —SO₂R¹³; R^{1a} is C₁-C₆ alkyl substituted with one or more hydroxy, and R^{1b} is —CONR¹¹R¹²; R^{1a} is C₁-C₆ alkyl substituted with one or more hydroxy, and R^{1b} is —OR¹¹; R^{1a} is C₁-C₆ alkyl substituted with one or more hydroxy, and R^{1b} is —COR¹³; R^{1a} is C₁-C₆ alkyl substituted with one or more hydroxy, and R^{1b} is —CO₂R¹³; R^{1a} is C₁-C₆ alkyl substituted with one or more hydroxy, and R^{1b} is —NR¹³CONR¹¹R¹²; R^{1a} is C₁-C₆ alkyl substituted with one or more hydroxy, and R^{1b} is —CR¹¹R¹²CN; R^{1a} is C₁-C₆ alkyl substituted with one or more hydroxy, and R^{1b} is —NR¹¹SO₂R¹³; R^{1a} is C₁-C₆ alkyl substituted with one or more hydroxy, and R^{1b} is —NR¹¹CONR¹¹R¹²; R^{1a} is C₁-C₆ alkyl substituted with one or more hydroxy, and R^{1b} is —CN; R^{1a} is C₁-C₆ alkyl substituted with one or more hydroxy, and R^{1b} is —NR¹¹COR¹²; R^{1a} is C₁-C₆ alkyl substituted with one or more hydroxy, and R^{1b} is —CR¹¹R¹²NR¹¹R¹²; R^{1a} is C₁-C₆ alkyl substituted with one or more —OSi(R¹³)₃, and R^{1b} is C₁-C₆ alkyl substituted with one or more hydroxy; R^{1a} is C₁-C₆ alkyl substituted with one or more —OSi(R¹³)₃, and R^{1b} is —SO₂NR¹¹R¹²; R^{1a} is C₁-C₆ alkyl substituted with one or more —OSi(R¹³)₃, and R^{1b} is —SO₂R¹³; R^{1a} is C₁-C₆ alkyl substituted with one or more —OSi(R¹³)₃, and R^{1b} is —CONR¹¹R¹²; R^{1a} is C₁-C₆ alkyl substituted with one or more —OSi(R¹³)₃, and R^{1b} is —OR¹¹; R^{1a} is C₁-C₆ alkyl substituted with one or more —OSi(R¹³)₃, and R^{1b} is —COR¹³; R^{1a} is C₁-C₆ alkyl substituted with one or more —OSi(R¹³)₃, and R^{1b} is —CO₂R¹³; R^{1a} is C₁-C₆ alkyl substituted with one or more —OSi(R¹³)₃, and R^{1b} is —NR¹³CONR¹¹R¹²; R^{1a} is C₁-C₆ alkyl substituted with one or more —OSi(R¹³)₃, and R^{1b} is —CR¹¹R¹²CN; R^{1a} is C₁-C₆ alkyl substituted with one or more —OSi(R¹³)₃, and R^{1b} is —NR¹¹SO₂R¹³; R^{1a} is C₁-C₆ alkyl substituted with one or more —OSi(R¹³)₃, and R^{1b} is —NR¹¹CONR¹¹R¹²; R^{1a} is C₁-C₆ alkyl substituted with one or more —OSi(R¹³)₃, and R^{1b} is —CN; R^{1a} is C₁-C₆ alkyl substituted with one or more —OSi(R¹³)₃, and R^{1b} is —NR¹¹COR¹²; R^{1a} is C₁-C₆ alkyl substituted with one or more —OSi(R¹³)₃, and R^{1b} is —CR¹¹R¹²NR¹¹R¹²; R^{1a} is —SO₂NR¹¹R¹², and R^{1b} is C₁-C₆ alkyl substituted with one or more hydroxy; R^{1a} is —SO₂NR¹¹R¹², and R^{1b} is —SO₂NR¹¹R¹²; R^{1a} is —SO₂NR¹¹R¹², and R^{1b} is —SO₂R¹³; R^{1a} is —SO₂NR¹¹R¹², and R^{1b} is —CONR¹¹R¹²; R^{1a} is —SO₂NR¹¹R¹², and R^{1b} is —OR¹¹; R^{1a} is —SO₂NR¹¹R¹², and R^{1b} is —COR¹³; R^{1a} is —SO₂NR¹¹R¹², and R^{1b} is —CO₂R¹³; R^{1a} is —SO₂NR¹¹R¹², and R^{1b} is —NR¹³CONR¹¹R¹²; R^{1a} is —SO₂NR¹¹R¹², and R^{1b} is —CN; R^{1a} is —SO₂NR¹¹R¹², and R^{1b} is —CR¹¹R¹²CN; R^{1a} is —SO₂NR¹¹R¹², and R^{1b} is —NR¹¹SO₂R¹³; R^{1a} is —SO₂NR¹¹R¹², and R^{1b} is —CR¹¹R¹²NR¹¹R¹²; R^{1a} is —SO₂NR¹¹R¹², and R^{1b} is —NR¹¹CONR¹¹R¹²; R^{1a} is —SO₂NR¹¹R¹², and R^{1b} is —NR¹¹COR¹²; R^{1a} is —CR¹¹R¹²NR¹¹R¹², and R^{1b} is C₁-C₆ alkyl substituted with one or more hydroxy; R^{1a} is —CR¹¹R¹²NR¹¹R¹², and R^{1b} is —SO₂NR¹¹R¹²; R^{1a} is —CR¹¹R¹²NR¹¹R¹², and R^{1b} is —SO₂R¹³; R^{1a} is —CR¹¹R¹²NR¹¹R¹², and R^{1b} is —CONR¹¹R¹²; R^{1a} is —CR¹¹R¹²NR¹¹R¹², and R^{1b} is —OR¹¹; R^{1a} is —CR¹¹R¹²NR¹¹R¹², and R^{1b} is —COR¹³; R^{1a} is —CR¹¹R¹²NR¹¹R¹², and R^{1b} is —CO₂R¹³; R^{1a} is —CR¹¹R¹²NR¹¹R¹², and R^{1b} is —NR¹³CONR¹¹R¹²; R^{1a} is —CR¹¹R¹²NR¹¹R¹², and R^{1b} is —CR¹¹R¹²CN; R^{1a} is —CR¹¹R¹²NR¹¹R¹², and R^{1b} is —NR¹¹SO₂R¹³; R^{1a} is —CR¹¹R¹²NR¹¹R¹², and R^{1b} is —CR¹¹R¹²NR¹¹R¹²; R^{1a} is —CR¹¹R¹²NR¹¹R¹², and R^{1b} is —CN; R^{1a} is —CR¹¹R¹²NR¹¹R¹², and R^{1b} is —NR¹¹CONR¹¹R¹²; and R^{1a} is —CR¹¹R¹²NR¹¹R¹², and R^{1b} is —NR¹¹COR¹².

In some embodiments of the compound of formula AA,

the substituted ring A is

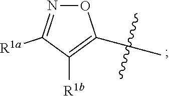

R^{1a} and R^{1b} are one of the following combinations:

In some embodiments, R^{1a} is 2-hydroxy-2-propyl, and R^{1b} is —OMe; In some embodiments, R^{1a} is 2-hydroxy-2-propyl, and R^{1b} is —OH; In some embodiments, R^{1a} is 2-hydroxy-2-propyl, and R^{1b} is —CO₂Me; In some embodiments, R^{1a} is 2-hydroxy-2-propyl, and R^{1b} is hydroxymethyl; In some embodiments, R^{1a} is 2-hydroxy-2-propyl, and R^{1b} is hydroxy ethyl; In some embodiments, R^{1a} is 2-hydroxy-2-propyl, and R^{1b} is 2-hydroxy-2-propyl; In some embodiments, R^{1a} is 2-hydroxy-2-propyl, and R^{1b} is —SO₂NHCH₂CH₂OH; In some embodiments, R^{1a} is 2-hydroxy-2-propyl, and R^{1b} is —SO₂Me; In some embodiments, R^{1a} is 2-hydroxy-2-propyl, and R^{1b} is CONHMe; In some embodiments, R^{1a} is 2-hydroxy-2-propyl, and R^{1b} is cyanomethyl; In some embodiments, R^{1a} is 2-hydroxy-2-propyl, and R^{1b} is —CN; In some embodiments, R^{1a} is 2-hydroxy-2-propyl, and R^{1b} is dimethylaminomethyl; In some embodiments, R^{1a} is dimethylaminomethyl, and R^{1b} is —OMe; In some embodiments, R^{1a} is dimethylaminomethyl, and R^{1b} is —OH; In some embodiments, R^{1a} is dimethylaminomethyl, and R^{1b} is —CO₂Me; In some embodiments, R^{1a} is dimethylaminomethyl, and R^{1b} is hydroxymethyl; In some embodiments, R^{1a} is dimethylaminomethyl, and R^{1b} is hydroxy ethyl; In some embodiments, R^{1a} is dimethylaminomethyl, and R^{1b} is 2-hydroxy-2-propyl; In some embodiments, R^{1a} is dimethylaminomethyl, and R^{1b} is —SO₂NHCH₂CH₂OH; In some embodiments, R^{1a} is dimethylaminomethyl, and R^{1b} is —SO₂Me; In some embodiments, R^{1a} is dimethylaminomethyl, and R^{1b} is CONHMe; In some embodiments, R^{1a} is dimethylaminomethyl, and R^{1b} is cyanomethyl; In some embodiments, R^{1a} is dimethylaminomethyl, and R^{1b} is dimethylaminomethyl; In some embodiments, R^{1a} is dimethylaminomethyl, and R^{1b} is CN. In some embodiments, R^{1a} is —SO₂NHMe, and R^{1b} is —OMe; In some embodiments, R^{1a} is —SO₂NHMe, and R^{1b} is —OH; In some embodiments, R^{1a} is —SO₂NHMe, and R^{1b} is —CO₂Me; In some embodiments, R^{1a} is —SO₂NHMe, and R^{1b} is hydroxymethyl; In some embodiments, R^{1a} is —SO₂NHMe, and R^{1b} is hydroxyethyl; In some embodiments, R^{1a} is —SO₂NHMe, and R^{1b} is 2-hydroxy-2-propyl; In some embodiments, R^{1a} is —SO₂NHMe, and R^{1b} is —SO₂NHCH₂CH₂OH; In some embodiments, R^{1a} is —SO₂NHMe, and R^{1b} is —SO₂Me; In some embodiments, R^{1a} is —SO₂NHMe, and R^{1b} is CONHMe; In some embodiments, R^{1a} is —SO₂NHMe, and R^{1b} is dimethylaminomethyl; In some embodiments, R^{1a} is —SO₂NHMe, and R^{1b} is —CN; In some embodiments, R^{1a} is —SO₂NHMe, and R^{1b} is cyanomethyl; and In some embodiments, R^{1a} is C₁-C₄ alkyl substituted with one —OSi(Me)₂tBu, and R^{1b} is —CO₂Me.

In some embodiments of the compound of formula AA,

the substituted ring A is

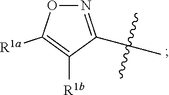

R^{1a} and R^{1b} are one of the following combinations:
R^{1a} is C₁-C₆ alkyl substituted with one or more hydroxy, and R^{1b} is C₁-C₆ alkyl substituted with one or more hydroxy; R^{1a} is C₁-C₆ alkyl substituted with one or more hydroxy, and R^{1b} is —SO₂NR¹¹R¹²; R^{1a} is C₁-C₆ alkyl substituted with one or more hydroxy, and R^{1b} is —SO₂R¹³; R^{1a} is C₁-C₆ alkyl substituted with one or more hydroxy, and R^{1b} is —CONR¹¹R¹²; R^{1a} is C₁-C₆ alkyl substituted with one or more hydroxy, and R^{1b} is —OR¹¹; R^{1a} is C₁-C₆ alkyl substituted with one or more hydroxy, and R^{1b} is —COR¹³; R^{1a} is C₁-C₆ alkyl substituted with one or more hydroxy, and R^{1b} is —CO₂R¹³; R^{1a} is C₁-C₆ alkyl substituted with one or more hydroxy, and R^{1b} is —NR¹³CONR¹¹R¹²; R^{1a} is C₁-C₆ alkyl substituted with one or more hydroxy, and R^{1b} is —CR¹¹R¹²CN; R^{1a} is C₁-C₆ alkyl substituted with one or more hydroxy, and R^{1b} is —NR¹¹SO₂R¹³; R^{1a} is C₁-C₆ alkyl substituted with one or more hydroxy, and R^{1b} is —NR¹¹CONR¹¹R¹²; R^{1a} is C₁-C₆ alkyl substituted with one or more hydroxy, and R^{1b} is —CN; R^{1a} is C₁-C₆ alkyl substituted with one or more hydroxy, and R^{1b} is —NR¹¹COR¹²; R^{1a} is C₁-C₆ alkyl substituted with one or more hydroxy, and R^{1b} is —CR¹¹R¹²NR¹¹R¹²; R^{1a} is C₁-C₆ alkyl substituted with one or more —OSi(R¹³)₃, and R^{1b} is C₁-C₆ alkyl substituted with one or more hydroxy; R^{1a} is C₁-C₆ alkyl substituted with one or more —OSi(R¹³)₃, and R^{1b} is —SO₂NR¹¹R¹²; R^{1a} is C₁-C₆ alkyl substituted with one or more —OSi(R¹³)₃, and R^{1b} is —SO₂R¹³; R^{1a} is C₁-C₆ alkyl substituted with one or more —OSi(R¹³)₃, and R^{1b} is —CONR¹¹R¹²; R^{1a} is C₁-C₆ alkyl substituted with one or more —OSi(R¹³)₃, and R^{1b} is —OR¹¹; R^{1a} is C₁-C₆ alkyl substituted with one or more —OSi(R¹³)₃, and R^{1b} is —COR¹³; R^{1a} is C₁-C₆ alkyl substituted with one or more —OSi(R¹³)₃, and R^{1b} is —CO₂R¹³; R^{1a} is C₁-C₆ alkyl substituted with one or more —OSi(R¹³)₃, and R^{1b} is —NR¹³CONR¹¹R¹²; R^{1a} is C₁-C₆ alkyl substituted with one or more —OSi(R¹³)₃, and R^{1b} is —CR¹¹R¹²CN; R^{1a} is C₁-C₆ alkyl substituted with one or more —OSi(R¹³)₃, and R^{1b} is —NR¹¹SO₂R¹³; R^{1a} is C₁-C₆ alkyl substituted with one or more —OSi(R¹³)₃, and R^{1b} is —NR¹¹CONR¹¹R¹²; R^{1a} is C₁-C₆ alkyl substituted with one or more —OSi(R¹³)₃, and R^{1b} is —CN; R^{1a} is C₁-C₆ alkyl substituted with one or more —OSi(R¹³)₃, and R^{1b} is —NR¹¹COR¹²; R^{1a} is C₁-C₆ alkyl substituted with one or more —OSi(R¹³)₃, and R^{1b} is —CR¹¹R¹²NR¹¹R¹²; R^{1a} is —SO₂NR¹¹R¹², and R^{1b} is C₁-C₆ alkyl substituted with one or more hydroxy; R^{1a} is —SO₂NR¹¹R¹², and R^{1b} is —SO₂NR¹¹R¹²; R^{1a} is —SO₂NR¹¹R¹², and R^{1b} is —SO₂R¹³; R^{1a} is —SO₂NR¹¹R¹², and R^{1b} is —CONR¹¹R¹²; R^{1a} is —SO₂NR¹¹R¹², and R^{1b} is —OR¹¹; R^{1a} is —SO₂NR¹¹R¹², and R^{1b} is —COR¹³; R^{1a} is —SO₂NR¹¹R¹², and R^{1b} is —CO₂R¹³; R^{1a} is —SO₂NR¹¹R¹², and R^{1b} is —NR¹³CONR¹¹R¹²; R^{1a} is —SO₂NR¹¹R¹², and R^{1b} is —CR¹¹R¹²CN; R^{1a} is —SO₂NR¹¹R¹², and R^{1b} is —NR¹¹SO₂R¹³; R^{1a} is —SO₂NR¹¹R¹², and R^{1b} is —CR¹¹R¹²NR¹¹R¹²; R^{1a} is —SO₂NR¹¹R¹², and R^{1b} is —CN; R^{1a} is —SO₂NR¹¹R¹², and R^{1b} is —NR¹¹CONR¹¹R¹²; R^{1a} is —SO₂NR¹¹R¹², and R^{1b} is —NR¹¹COR¹²; R^{1a} is —CR¹¹R¹²NR¹¹R¹², and R^{1b} is C₁-C₆ alkyl substituted with one or more hydroxy; R^{1a} is —CR¹¹R¹²NR¹¹R¹², and R^{1b} is —SO₂NR¹¹R¹²; R^{1a} is —CR¹¹R¹²NR¹¹R¹², and R^{1b} is —SO₂R¹³; R^{1a} is —CR¹¹R¹²NR¹¹R¹², and R^{1b} is —CONR¹¹R¹²; R^{1a} is —CR¹¹R¹²NR¹¹R¹², and R^{1b} is —OR¹¹; R^{1a} is —CR¹¹R¹²NR¹¹R¹², and R^{1b} is —COR¹³; R^{1a} is —CR¹¹R¹²NR¹¹R¹², and R^{1b} is —CO₂R¹³; R^{1a} is —CR¹¹R¹²NR¹¹R¹², and R^{1b} is —NR¹³CONR¹¹R¹²; R^{1a} is —CR¹¹R¹²NR¹¹R¹², and R^{1b} is —CR¹¹R¹²CN; R^{1a} is —CR¹¹R¹²NR¹¹R¹², and R^{1b} is —NR¹¹SO₂R¹³; R^{1a} is —CR¹¹R¹²NR¹¹R¹², and R^{1b} is —CR¹¹R¹²NR¹¹R¹²; R^{1a} is —CR¹¹R¹²NR¹¹R¹², and R^{1b} is —CN; R^{1a} is —CR¹¹R¹²NR¹¹R¹², and R^{1b} is —NR¹¹CONR¹¹R¹²; and R^{1a} is —CR¹¹R¹²NR¹¹R¹², and R^{1b} is —NR¹¹COR¹².

In some embodiments of the compound of formula AA,

the substituted ring A is

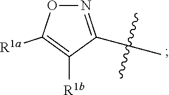

R^{1a} and R^{1b} are one of the following combinations:

In some embodiments, R^{1a} is 2-hydroxy-2-propyl, and R^{1b} is —OMe; In some embodiments, R^{1a} is 2-hydroxy-2-propyl, and R^{1b} is —OH; In some embodiments, R^{1a} is 2-hydroxy-2-propyl, and R^{1b} is —CO₂Me; In some embodiments, R^{1a} is 2-hydroxy-2-propyl, and R^{1b} is hydroxymethyl; In some embodiments, R^{1a} is 2-hydroxy-2-propyl, and R^{1b} is hydroxy ethyl; In some embodiments, R^{1a} is 2-hydroxy-2-propyl, and R^{1b} is 2-hydroxy-2-propyl; In some embodiments, R^{1a} is 2-hydroxy-2-propyl, and R^{1b} is —SO₂NHCH₂CH₂OH; In some embodiments, R^{1a} is 2-hydroxy-2-propyl, and R^{1b} is —SO₂Me; In some embodiments, R^{1a} is 2-hydroxy-2-propyl, and R^{1b} is CONHMe; In some embodiments, R^{1a} is 2-hydroxy-2-propyl, and R^{1b} is cyanomethyl; In some embodiments, R^{1a} is 2-hydroxy-2-propyl, and R^{1b} is —CN; In some embodiments, R^{1a} is 2-hydroxy-2-propyl, and R^{1b} is dimethylaminomethyl; In some embodiments, R^{1a} is dimethylaminomethyl, and R^{1b} is —OMe; In some embodiments, R^{1a} is dimethylaminomethyl, and R^{1b} is —OH; In some embodiments, R^{1a} is dimethylaminomethyl, and R^{1b} is —CO₂Me; In some embodiments, R^{1a} is dimethylaminomethyl, and R^{1b} is hydroxymethyl; In some embodiments, R^{1a} is dimethylaminomethyl, and R^{1b} is hydroxy ethyl; In some embodiments, R^{1a} is dimethylaminomethyl, and R^{1b} is 2-hydroxy-2-propyl; In some embodiments, R^{1a} is dimethylaminomethyl, and R^{1b} is —SO₂NHCH₂CH₂OH; In some embodiments, R^{1a} is dimethylaminomethyl, and R^{1b} is —SO₂Me; In some embodiments, R^{1a} is dimethylaminomethyl, and R^{1b} is CONHMe; In some embodiments, R^{1a} is dimethylaminomethyl, and R^{1b} is cyanomethyl; In some embodiments, R^{1a} is dimethylaminomethyl, and R^{1b} is dimethylaminomethyl; In some embodiments, R^{1a} is dimethylaminomethyl, and R^{1b} is CN. In some embodiments, R^{1a} is —SO₂NHMe, and R^{1b} is —OMe; In some embodiments, R^{1a} is —SO₂NHMe, and R^{1b} is —OH; In some embodiments, R^{1a} is —SO₂NHMe, and R^{1b} is —CO₂Me; In some embodiments, R^{1a} is —SO₂NHMe, and R^{1b} is hydroxymethyl; In some embodiments, R^{1a} is —SO₂NHMe, and R^{1b} is hydroxyethyl; In some embodiments, R^{1a} is —SO₂NHMe, and R^{1b} is 2-hydroxy-2-propyl; In some embodiments, R^{1a} is —SO₂NHMe, and R^{1b} is —SO₂NHCH₂CH₂OH; In some embodiments, R^{1a} is —SO₂NHMe, and R^{1b} is —SO₂Me; In some embodiments, R^{1a} is —SO₂NHMe, and R^{1b} is CONHMe; In some embodiments, R^{1a} is —SO₂NHMe, and R^{1b} is dimethylaminomethyl; In some embodiments, R^{1a} is —SO₂NHMe, and R^{1b} is —CN; In some embodiments, R^{1a} is —SO₂NHMe, and R^{1b} is cyanomethyl; and In some embodiments, R^{1a} is C₁-C₄ alkyl substituted with one —OSi(Me)₂tBu, and R^{1b} is —CO₂Me.

In some embodiments of the compound of formula AA,

the substituted ring A is

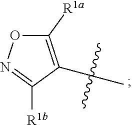

R^{1a} and R^{1b} are one of the following combinations:
R^{1a} is C₁-C₆ alkyl substituted with one or more hydroxy, and R^{1b} is C₁-C₆ alkyl substituted with one or more hydroxy; R^{1a} is C₁-C₆ alkyl substituted with one or more hydroxy, and R^{1b} is —SO₂NR¹¹R¹²; R^{1a} is C₁-C₆ alkyl substituted with one or more hydroxy, and R^{1b} is —SO₂R¹³; R^{1a} is C₁-C₆ alkyl substituted with one or more hydroxy, and R^{1b} is —CONR¹¹R¹²; R^{1a} is C₁-C₆ alkyl substituted with one or more hydroxy, and R^{1b} is —OR¹¹; R^{1a} is C₁-C₆ alkyl substituted with one or more hydroxy, and R^{1b} is —COR¹³; R^{1a} is C₁-C₆ alkyl substituted with one or more hydroxy, and R^{1b} is —CO₂R¹³; R^{1a} is C₁-C₆ alkyl substituted with one or more hydroxy, and R^{1b} is —NR¹³CONR¹¹R¹²; R^{1a} is C₁-C₆ alkyl substituted with one or more hydroxy, and R^{1b} is —CR¹¹R¹²CN; R^{1a} is C₁-C₆ alkyl substituted with one or more hydroxy, and R^{1b} is —NR¹¹SO₂R¹³; R^{1a} is C₁-C₆ alkyl substituted with one or more hydroxy, and R^{1b} is —NR¹¹CONR¹¹R¹²; R^{1a} is C₁-C₆ alkyl substituted with one or more hydroxy, and R^{1b} is —NR¹¹COR¹²; R^{1a} is C₁-C₆ alkyl substituted with one or more hydroxy, and R^{1b} is —CR¹¹R¹²NR¹¹R¹²; R^{1a} is C₁-C₆ alkyl substituted with one or more —OSi(R¹³)₃, and R^{1b} is —SO₂NR¹¹R¹²; R^{1a} is C₁-C₆ alkyl substituted with one or more —OSi(R¹³)₃, and R^{1b} is —SO₂R¹³; R^{1a} is C₁-C₆ alkyl substituted with one or more —OSi(R¹³)₃, and R^{1b} is —CONR¹¹R¹²; R^{1a} is C₁-C₆ alkyl substituted with one or more —OSi(R¹³)₃, and R^{1b} is —OR¹¹; R^{1a} is C₁-C₆ alkyl substituted with one or more —OSi(R¹³)₃, and R^{1b} is —COR¹³; R^{1a} is C₁-C₆ alkyl substituted with one or more —OSi(R¹³)₃, and R^{1b} is —CO₂R¹³; R^{1a} is C₁-C₆ alkyl substituted with one or more —OSi(R¹³)₃, and R^{1b} is —NR¹³CONR¹¹R¹²; R^{1a} is C₁-C₆ alkyl substituted with one or more —OSi(R¹³)₃, and R^{1b} is —CR¹¹R¹²CN; R^{1a} is C₁-C₆ alkyl substituted with one or more —OSi(R¹³)₃, and R^{1b} is —NR¹¹SO₂R¹³; R^{1a} is C₁-C₆ alkyl substituted with one or more —OSi(R¹³)₃, and R^{1b} is —NR¹¹CONR¹¹R¹²; R^{1a} is C₁-C₆ alkyl substituted with one or more hydroxy, and R^{1b} is —CN; R^{1a} is C₁-C₆ alkyl substituted with one or more —OSi(R¹³)₃, and R^{1b} is —NR¹¹COR¹²; R^{1a} is C₁-C₆ alkyl substituted with one or more —OSi(R¹³)₃, and R^{1b} is —CR¹¹R¹²NR¹¹R¹²; R^{1a} is C₁-C₆ alkyl substituted with one or more —OSi(R¹³)₃, and R^{1b} is C₁-C₆ alkyl substituted with one or more hydroxy; R^{1a} is —SO₂NR¹¹R¹², and R^{1b} is —SO₂NR¹¹R¹²; R^{1a} is —SO₂NR¹¹R¹², and R^{1b} is —SO₂R¹³; R^{1a} is —SO₂NR¹¹R¹², and R^{1b} is —CONR¹¹R¹²; R^{1a} is —SO₂NR¹¹R¹², and R^{1b} is —OR¹¹; R^{1a} is —SO₂NR¹¹R¹², and R^{1b} is —COR¹³; R^{1a} is —SO₂NR¹¹R¹², and R^{1b} is —CO₂R¹³; R^{1a} is —SO₂NR¹¹R¹², and R^{1b} is —NR¹³CONR¹¹R¹²; R^{1a} is —SO₂NR¹¹R¹², and R^{1b} is —CR¹¹R¹²CN; R^{1a} is —SO₂NR¹¹R¹², and R^{1b} is —NR¹¹SO₂R¹³; R^{1a} is —SO₂NR¹¹R¹², and R^{1b} is —CR¹¹R¹²NR¹¹R¹²; R^{1a} is —SO₂NR¹¹R¹², and R^{1b} is —CN; R^{1a} is —SO₂NR¹¹R¹², and R^{1b} is —NR¹¹CONR¹¹R¹²; R^{1a} is —SO₂NR¹¹R¹², and R^{1b} is —NR¹¹COR¹²; R^{1a} is —CR¹¹R¹²NR¹¹R¹², and R^{1b} is C₁-C₆ alkyl substituted with one or more hydroxy; R^{1a} is —CR¹¹R¹²NR₁₁R¹², and R^{1b} is —SO₂NR¹¹R¹²; R^{1a} is —CR¹¹R¹²NR¹¹R¹², and R^{1b} is —SO₂R¹³; R^{1a} is —CR¹¹R¹²NR¹¹R¹², and R^{1b} is —CONR¹¹R¹²; R^{1a} is —CR¹¹R¹²NR¹¹R¹², and R^{1b} is —OR¹¹; R^{1a} is —CR¹¹R¹²NR¹¹R¹², and R^{1b} is —COR¹³; R^{1a} is —CR¹¹R¹²NR¹¹R¹², and R^{1b} is —CO₂R¹³; R^{1a} is —CR¹¹R¹²NR¹¹R¹², and R^{1b} is —NR¹³CONR¹¹R¹²; R^{1a} is —CR¹¹R¹²NR¹¹R¹², and R^{1b} is —CR¹¹R¹²CN; R^{1a} is —CR¹¹R¹²NR¹¹R¹², and R^{1b} is —NR¹¹SO₂R¹³; R^{1a} is —CR¹¹R¹²NR¹¹R¹², and R^{1b} is —CR¹¹R¹²NR¹¹R¹²; R^{1a} is —CR¹¹R¹²NR¹¹R¹², and R^{1b} is —CN; R^{1a} is —CR¹¹R¹²NR¹¹R¹², and R^{1b} is —NR¹¹CONR¹¹R¹²; and R^{1a} is —CR¹¹R¹²NR¹¹R¹², and R^{1b} is —NR¹¹COR¹².

In some embodiments of the compound of formula AA,

the substituted ring A is

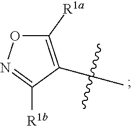

R^{1a} and R^{1b} are one of the following combinations:

In some embodiments, R^{1a} is 2-hydroxy-2-propyl, and R^{1b} is —OMe; In some embodiments, R^{1a} is 2-hydroxy-2-propyl, and R^{1b} is —OH; In some embodiments, R^{1a} is 2-hydroxy-2-propyl, and R^{1b} is —CO₂Me; In some embodiments, R^{1a} is 2-hydroxy-2-propyl, and R^{1b} is hydroxymethyl; In some embodiments, R^{1a} is 2-hydroxy-2-propyl, and R^{1b} is hydroxy ethyl; In some embodiments, R^{1a} is 2-hydroxy-2-propyl, and R^{1b} is 2-hydroxy-2-propyl; In some embodiments, R^{1a} is 2-hydroxy-2-propyl, and R^{1b} is —SO₂NHCH₂CH₂OH; In some embodiments, R^{1a} is 2-hydroxy-2-propyl, and R^{1b} is —SO₂Me; In some embodiments, R^{1a} is 2-hydroxy-2-propyl, and R^{1b} is CONHMe; In some embodiments, R^{1a} is 2-hydroxy-2-propyl, and R^{1b} is cyanomethyl; In some embodiments, R^{1a} is 2-hydroxy-2-propyl, and R^{1b} is —CN; In some embodiments, R^{1a} is 2-hydroxy-2-propyl, and R^{1b} is dimethylaminomethyl; In some embodiments, R^{1a} is dimethylaminomethyl, and R^{1b} is —OMe; In some embodiments, R^{1a} is dimethylaminomethyl, and R^{1b} is —OH; In some embodiments, R^{1a} is dimethylaminomethyl, and R^{1b} is —CO₂Me; In some embodiments, R^{1a} is dimethylaminomethyl, and R^{1b} is hydroxymethyl; In some embodiments, R^{1a} is dimethylaminomethyl, and R^{1b} is hydroxy ethyl; In some embodiments, R^{1a} is dimethylaminomethyl, and R^{1b} is 2-hydroxy-2-propyl; In some embodiments, R^{1a} is dimethylaminomethyl, and R^{1b} is —SO₂NHCH₂CH₂OH; In some embodiments, R^{1a} is dimethylaminomethyl, and R^{1b} is —SO₂Me; In some embodiments, R^{1a} is dimethylaminomethyl, and R^{1b} is CONHMe; In some embodiments, R^{1a} is dimethylaminomethyl, and R^{1b} is cyanomethyl; In some embodiments, R^{1a} is dimethylaminomethyl, and R^{1b} is dimethylaminomethyl; In some embodiments, R^{1a} is dimethylaminomethyl, and R^{1b} is CN; In some embodiments, R^{1a} is —SO₂NHMe, and R^{1b} is —OMe; In some embodiments, R^{1a} is —SO₂NHMe, and R^{1b} is —OH; In some embodiments, R^{1a} is —SO₂NHMe, and R^{1b} is —CO₂Me; In some embodiments, R^{1a} is —SO₂NHMe, and R^{1b} is hydroxymethyl; In some embodiments, R^{1a} is —SO₂NHMe, and R^{1b} is hydroxyethyl; In some embodiments, R^{1a} is —SO₂NHMe, and R^{1b} is 2-hydroxy-2-propyl; In some embodiments, R^{1a} is —SO₂NHMe, and R^{1b} is —SO₂NHCH₂CH₂OH; In some embodiments, R^{1a} is —SO₂NHMe, and R^{1b} is —SO₂Me; In some embodiments, R^{1a} is —SO₂NHMe, and R^{1b} is CONHMe; In some embodiments, R^{1a} is —SO₂NHMe, and R^{1b} is dimethylaminomethyl; In some embodiments, R^{1a} is —SO₂NHMe, and R^{1b} is —CN; In some embodiments, R^{1a} is —SO₂NHMe, and R^{1b} is cyanomethyl; and In some embodiments, R^{1a} is C₁-C₄ alkyl substituted with one —OSi(Me)₂tBu, and R^{1b} is —CO₂Me.

In some embodiments of the compound of formula AA,

the substituted ring A is

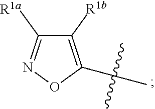

R^{1a} and R^{1b} are one of the following combinations:
R^{1a} is C₁-C₆ alkyl substituted with one or more hydroxy, and R^{1b} is C₁-C₆ alkyl substituted with one or more hydroxy; R^{1a} is C₁-C₆ alkyl substituted with one or more hydroxy, and R^{1b} is —SO₂NR¹¹R¹²; R^{1a} is C₁-C₆ alkyl substituted with one or more hydroxy, and R^{1b} is —SO₂R¹³; R^{1a} is C₁-C₆ alkyl substituted with one or more hydroxy, and R^{1b} is —CONR¹¹R¹²; R^{1a} is C₁-C₆ alkyl substituted with one or more hydroxy, and R^{1b} is —OR¹¹; R^{1a} is C₁-C₆ alkyl substituted with one or more hydroxy, and R^{1b} is —COR¹³; R^{1a} is C₁-C₆ alkyl substituted with one or more hydroxy, and R^{1b} is —CO₂R¹³; R^{1a} is C₁-C₆ alkyl substituted with one or more hydroxy, and R^{1b} is —NR¹³CONR¹¹R¹²; R^{1a} is C₁-C₆ alkyl substituted with one or more hydroxy, and R^{1b} is —CR¹¹R¹²CN; R^{1a} is C₁-C₆ alkyl substituted with one or more hydroxy, and R^{1b} is —NR¹¹SO₂R¹³; R^{1a} is C₁-C₆ alkyl substituted with one or more hydroxy, and R^{1b} is —NR¹¹CONR¹¹R¹²; R^{1a} is C₁-C₆ alkyl substituted with one or more hydroxy, and R^{1b} is —CN; R^{1a} is C₁-C₆ alkyl substituted with one or more hydroxy, and R^{1b} is —NR¹¹COR¹²; R^{1a} is C₁-C₆ alkyl substituted with one or more hydroxy, and R^{1b} is —CR¹¹R¹²NR¹¹R¹²; R^{1a} is C₁-C₆ alkyl substituted with one or more —OSi(R¹³)₃, and R^{1b} is C₁-C₆ alkyl substituted with one or more hydroxy; R^{1a} is C₁-C₆ alkyl substituted with one or more —OSi(R¹³)₃, and R^{1b} is —SO₂NR¹¹R¹²; R^{1a} is C₁-C₆ alkyl substituted with one or more —OSi(R¹³)₃, and R^{1b} is —SO₂R¹³; R^{1a} is C₁-C₆ alkyl substituted with one or more —OSi(R¹³)₃, and R^{1b} is —CONR¹¹R¹²; R^{1a} is C₁-C₆ alkyl substituted with one or more —OSi(R¹³)₃, and R^{1b} is —OR¹¹; R^{1a} is C₁-C₆ alkyl substituted with one or more —OSi(R¹³)₃, and R^{1b} is —COR¹³; R^{1a} is C₁-C₆ alkyl substituted with one or more —OSi(R¹³)₃, and R^{1b} is —CO₂R¹³; R^{1a} is C₁-C₆ alkyl substituted with one or more —OSi(R¹³)₃, and R^{1b} is —NR¹³CONR¹¹R¹²; R^{1a} is C₁-C₆ alkyl substituted with one or more —OSi(R¹³)₃, and R^{1b} is —CR¹¹R¹²CN; R^{1a} is C₁-C₆ alkyl substituted with one or more —OSi(R¹³)₃, and R^{1b} is —NR¹¹SO₂R¹³; R^{1a} is C₁-C₆ alkyl substituted with one or more —OSi(R¹³)₃, and R^{1b} is —NR¹¹CONR¹¹R¹²; R^{1a} is C₁-C₆ alkyl substituted with one or more —OSi(R¹³)₃, and R^{1b} is —CN; R^{1a} is C₁-C₆ alkyl substituted with one or more —OSi(R¹³)₃, and R^{1b} is —NR¹¹COR¹²; R^{1a} is C₁-C₆ alkyl substituted with one or more —OSi(R¹³)₃, and R^{1b} is —CR¹¹R¹²NR¹¹R¹²; R^{1a} is —SO₂NR¹¹R¹², and R^{1b} is C₁-C₆ alkyl substituted with one or more hydroxyl; R^{1a} is —SO₂NR¹¹R¹², and R^{1b} is —SO₂NR¹¹R¹²; R^{1a} is —SO₂NR¹¹R¹², and R^{1b} is —SO₂R¹³; R^{1a} is —SO₂NR¹¹R¹², and R^{1b} is —CONR¹¹R¹²; R^{1a} is —SO₂NR¹¹R¹², and R^{1b} is —OR¹¹; R^{1a} is —SO₂NR¹¹R¹², and R^{1b} is —COR¹³; R^{1a} is —SO₂NR¹¹R¹², and R^{1b} is —CO₂R¹³; R^{1a} is —SO₂NR¹¹R¹², and R^{1b} is —NR¹³CONR¹¹R¹²; R^{1a} is —SO₂NR¹¹R¹², and R^{1b} is —CR¹¹R¹²CN; R^{1a} is —SO₂NR¹¹R¹², and R^{1b} is —NR¹¹SO₂R¹³; R^{1a} is —SO₂NR¹¹R¹², and R^{1b} is —CR¹¹R¹²NR¹¹R¹²; R^{1a} is —SO₂NR¹¹R¹², and R^{1b} is —CN; R^{1a} is —SO₂NR¹¹R¹², and R^{1b} is —NR¹¹CONR¹¹R¹²; R^{1a} is —SO₂NR¹¹R¹², and R^{1b} is —NR¹¹COR¹²; R^{1a} is —CR¹¹R¹²NR¹¹R¹², and R^{1b} is C₁-C₆ alkyl substituted with one or more hydroxy; R^{1a} is —CR¹¹R¹²NR¹¹R¹², and R^{1b} is —SO₂NR¹¹R¹²; R^{1a} is —CR¹¹R¹²NR¹¹R¹², and R^{1b} is —SO₂R¹³; R^{1a} is —CR¹¹R¹²NR¹¹R¹², and R^{1b} is —CONR¹¹R¹²; R^{1a} is —CR¹¹R¹²NR¹¹R¹², and R^{1b} is —OR¹¹; R^{1a} is —CR¹¹R¹²NR¹¹R¹², and R^{1b} is —COR¹³; R^{1a} is —CR¹¹R¹²NR¹¹R¹², and R^{1b} is —CO₂R¹³; R^{1a} is —CR¹¹R¹²NR¹¹R¹², and R^{1b} is —NR¹³CONR¹¹R¹²; R^{1a} is —CR¹¹R¹²NR¹¹R¹², and R^{1b} is —CR¹¹R¹²CN; R^{1a} is —CR¹¹R¹²NR¹¹R¹², and R^{1b} is —NR¹¹SO₂R¹³; R^{1a} is —CR¹¹R¹²NR¹¹R¹², and R^{1b} is —CR¹¹R¹²NR¹¹R¹²; R^{1a} is —CR¹¹R¹²NR¹¹R¹², and R^{1b} is —CN; R^{1a} is —CR¹¹R¹²NR¹¹R¹², and R^{1b} is —NR¹¹CONR¹¹R¹²; and R^{1a} is —CR¹¹R¹²NR¹¹R¹², and R^{1b} is —NR¹¹COR¹².

In some embodiments of the compound of formula AA,

the substituted ring A is

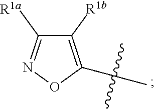

R^{1a} and R^{1b} are one of the following combinations:

In some embodiments, R^{1a} is 2-hydroxy-2-propyl, and R^{1b} is —OMe; In some embodiments, R^{1a} is 2-hydroxy-2-propyl, and R^{1b} is —OH; In some embodiments, R^{1a} is 2-hydroxy-2-propyl, and R^{1b} is —CO₂Me; In some embodiments, R^{1a} is 2-hydroxy-2-propyl, and R^{1b} is hydroxymethyl; In some embodiments, R^{1a} is 2-hydroxy-2-propyl, and R^{1b} is hydroxy ethyl; In some embodiments, R^{1a} is 2-hydroxy-2-propyl, and R^{1b} is 2-hydroxy-2-propyl; In some embodiments, R^{1a} is 2-hydroxy-2-propyl, and R^{1b} is —SO₂NHCH₂CH₂OH; In some embodiments, R^{1a} is 2-hydroxy-2-propyl, and R^{1b} is —SO₂Me; In some embodiments, R^{1a} is 2-hydroxy-2-propyl, and R^{1b} is CONHMe; In some embodiments, R^{1a} is 2-hydroxy-2-propyl, and R^{1b} is cyanomethyl; In some embodiments, R^{1a} is 2-hydroxy-2-propyl, and R^{1b} is —CN; In some embodiments, R^{1a} is 2-hydroxy-2-propyl, and R^{1b} is dimethylaminomethyl; In some embodiments, R^{1a} is dimethylaminomethyl, and R^{1b} is —OMe; In some embodiments, R^{1a} is dimethylaminomethyl, and R^{1b} is —OH; In some embodiments, R^{1a} is dimethylaminomethyl, and R^{1b} is —CO₂Me; In some embodiments, R^{1a} is dimethylaminomethyl, and R^{1b} is hydroxymethyl; In some embodiments, R^{1a} is dimethylaminomethyl, and R^{1b} is hydroxy ethyl; In some embodiments, R^{1a} is dimethylaminomethyl, and R^{1b} is 2-hydroxy-2-propyl; In some embodiments, R^{1a} is dimethylaminomethyl, and R^{1b} is —SO₂NHCH₂CH₂OH; In some embodiments, R^{1a} is dimethylaminomethyl, and R^{1b} is —SO₂Me; In some embodiments, R^{1a} is dimethylaminomethyl, and R^{1b} is CONHMe; In some embodiments, R^{1a} is dimethylaminomethyl, and R^{1b} is cyanomethyl; In some embodiments, R^{1a} is dimethylaminomethyl, and R^{1b} is dimethylaminomethyl; In some embodiments, R^{1a} is dimethylaminomethyl, and R^{1b} is CN; In some embodiments, R^{1a} is —SO₂NHMe, and R^{1b} is —OMe; In some embodiments, R^{1a} is —SO₂NHMe, and R^{1b} is —OH; In some embodiments, R^{1a} is —SO₂NHMe, and R^{1b} is —CO₂Me; In some embodiments, R^{1a} is —SO₂NHMe, and R^{1b} is hydroxymethyl; In some embodiments, R^{1a} is —SO₂NHMe, and R^{1b} is hydroxyethyl; In some embodiments, R^{1a} is —SO₂NHMe, and R^{1b} is 2-hydroxy-2-propyl; In some embodiments, R^{1a} is —SO₂NHMe, and R^{1b} is —SO₂NHCH₂CH₂OH; In some embodiments, R^{1a} is —SO₂NHMe, and R^{1b} is —SO₂Me; In some embodiments, R^{1a} is —SO₂NHMe, and R^{1b} is CONHMe; In some embodiments, R^{1a} is —SO₂NHMe, and R^{1b} is dimethylaminomethyl; In some embodiments, R^{1a} is —SO₂NHMe, and R^{1b} is —CN; In some embodiments, R^{1a} is —SO₂NHMe, and R^{1b} is cyanomethyl; and In some embodiments, R^{1a} is C₁-C₄ alkyl substituted with one —OSi(Me)₂tBu, and R^{1b} is —CO₂Me.

In some embodiments of the compound of formula AA,

the substituted ring A is

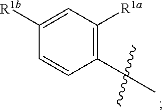

R^{1a} and R^{1b} are one of the following combinations:

In some embodiments, R^{1a} is 2-hydroxy-2-propyl, and R^{1b} is —OMe; In some embodiments, R^{1a} is 2-hydroxy-2-propyl, and R^{1b} is —OH; In some embodiments, R^{1a} is 2-hydroxy-2-propyl, and R^{1b} is —CO₂Me; In some embodiments, R^{1a} is 2-hydroxy-2-propyl, and R^{1b} is hydroxymethyl; In some embodiments, R^{1a} is 2-hydroxy-2-propyl, and R^{1b} is hydroxy ethyl; In some embodiments, R^{1a} is 2-hydroxy-2-propyl, and R^{1b} is 2-hydroxy-2-propyl; In some embodiments, R^{1a} is 2-hydroxy-2-propyl, and R^{1b} is —SO₂NHCH₂CH₂OH; In some embodiments, R^{1a} is 2-hydroxy-2-propyl, and R^{1b} is —SO₂Me; In some embodiments, R^{1a} is 2-hydroxy-2-propyl, and R^{1b} is CONHMe; In some embodiments, R^{1a} is 2-hydroxy-2-propyl, and R^{1b} is cyanomethyl; In some embodiments, R^{1a} is 2-hydroxy-2-propyl, and R^{1b} is —CN; In some embodiments, R^{1a} is 2-hydroxy-2-propyl, and R^{1b} is dimethylaminomethyl; In some embodiments, R^{1a} is dimethylaminomethyl, and R^{1b} is —OMe; In some embodiments, R^{1a} is dimethylaminomethyl, and R^{1b} is —OH; In some embodiments, R^{1a} is dimethylaminomethyl, and R^{1b} is —CO₂Me; In some embodiments, R^{1a} is dimethylaminomethyl, and R^{1b} is hydroxymethyl; In some embodiments, R^{1a} is dimethylaminomethyl, and R^{1b} is hydroxy ethyl; In some embodiments, R^{1a} is dimethylaminomethyl, and R^{1b} is 2-hydroxy-2-propyl; In some embodiments, R^{1a} is dimethylaminomethyl, and R^{1b} is —SO₂NHCH₂CH₂OH; In some embodiments, R^{1a} is dimethylaminomethyl, and R^{1b} is —SO₂Me; In some embodiments, R^{1a} is dimethylaminomethyl, and R^{1b} is CONHMe; In some embodiments, R^{1a} is dimethylaminomethyl, and R^{1b} is cyanomethyl; In some embodiments, R^{1a} is dimethylaminomethyl, and R^{1b} is dimethylaminomethyl; In some embodiments, R^{1a} is dimethylaminomethyl, and R^{1b} is CN; In some embodiments, R^{1a} is —SO₂NHMe, and R^{1b} is —OMe; In some embodiments, R^{1a} is —SO₂NHMe, and R^{1b} is —OH; In some embodiments, R^{1a} is —SO₂NHMe, and R^{1b} is —CO₂Me; In some embodiments, R^{1a} is —SO₂NHMe, and R^{1b} is hydroxymethyl; In some embodiments, R^{1a} is —SO₂NHMe, and R^{1b} is hydroxyethyl; In some embodiments, R^{1a} is —SO₂NHMe, and R^{1b} is 2-hydroxy-2-propyl; In some embodiments, R^{1a} is —SO₂NHMe, and R^{1b} is —SO₂NHCH₂CH₂OH; In some embodiments, R^{1a} is —SO₂NHMe, and R^{1b} is —SO₂Me; In some embodiments, R^{1a} is —SO₂NHMe, and R^{1b} is CONHMe; In some embodiments, R^{1a} is —SO₂NHMe, and R^{1b} is dimethylaminomethyl; In some embodiments, R^{1a} is —SO₂NHMe, and R^{1b} is —CN; In some embodiments, R^{1a} is —SO₂NHMe, and R^{1b} is cyanomethyl; and In some embodiments, R^{1a} is C₁-C₄ alkyl substituted with one —OSi(Me)₂tBu, and R^{1b} is —CO₂Me.

In some embodiments of the compound of formula AA,

the substituted ring A is

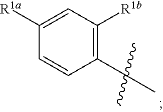

R^{1a} and R^{1b} are one of the following combinations:
R^{1a} is C₁-C₆ alkyl substituted with one or more hydroxy, and R^{1b} is C₁-C₆ alkyl substituted with one or more hydroxy; R^{1a} is C₁-C₆ alkyl substituted with one or more hydroxy, and R^{1b} is —SO₂NR¹¹R¹²; R^{1a} is C₁-C₆ alkyl substituted with one or more hydroxy, and R^{1b} is —SO₂R¹³; R^{1a} is C₁-C₆ alkyl substituted with one or more hydroxy, and R^{1b} is —CONR¹¹R¹²; R^{1a} is C₁-C₆ alkyl substituted with one or more hydroxy, and R^{1b} is —OR¹¹; R^{1a} is C₁-C₆ alkyl substituted with one or more hydroxy, and R^{1b} is —COR¹³; R^{1a} is C₁-C₆ alkyl substituted with one or more hydroxy, and R^{1b} is —CO₂R¹³; R^{1a} is C₁-C₆ alkyl substituted with one or more hydroxy, and R^{1b} is —NR¹³CONR¹¹R¹²; R^{1a} is C₁-C₆ alkyl substituted with one or more hydroxy, and R^{1b} is —CR¹¹R¹²CN; R^{1a} is C₁-C₆ alkyl substituted with one or more hydroxy, and R^{1b} is —NR¹¹SO₂R¹³; R^{1a} is C₁-C₆ alkyl substituted with one or more hydroxy, and R^{1b} is —NR¹¹CONR¹¹R¹²; R^{1a} is C₁-C₆ alkyl substituted with one or more hydroxy, and R^{1b} is —CN; R^{1a} is C₁-C₆ alkyl substituted with one or more hydroxy, and R^{1b} is —NR¹¹COR¹²; R^{1a} is C₁-C₆ alkyl substituted with one or more hydroxy, and R^{1b} is —CR¹¹R¹²NR¹¹R¹²; R^{1a} is C₁-C₆ alkyl substituted with one or more —OSi(R¹³)₃, and R^{1b} is C₁-C₆ alkyl substituted with one or more hydroxy; R^{1a} is C₁-C₆ alkyl substituted with one or more —OSi(R¹³)₃, and R^{1b} is —SO₂NR¹¹R¹²; R^{1a} is C₁-C₆ alkyl substituted with one or more —OSi(R¹³)₃, and R^{1b} is —SO₂R¹³; R^{1a} is C₁-C₆ alkyl substituted with one or more —OSi(R¹³)₃, and R^{1b} is —CONR¹¹R¹²; R^{1a} is C₁-C₆ alkyl substituted with one or more —OSi(R¹³)₃, and R^{1b} is —OR¹¹; R^{1a} is C₁-C₆ alkyl substituted with one or more —OSi(R¹³)₃, and R^{1b} is —COR¹³; R^{1a} is C₁-C₆ alkyl substituted with one or more —OSi(R¹³)₃, and R^{1b} is —CO₂R¹³; R^{1a} is C₁-C₆ alkyl substituted with one or more —OSi(R¹³)₃, and R^{1b} is —NR¹³CONR¹¹R¹²; R^{1a} is C₁-C₆ alkyl substituted with one or more —OSi(R¹³)₃, and R^{1b} is —CR¹¹R¹²CN; R^{1a} is C₁-C₆ alkyl substituted with one or more —OSi(R¹³)₃, and R^{1b} is —NR¹¹SO₂R¹³; R^{1a} is C₁-C₆ alkyl substituted with one or more —OSi(R¹³)₃, and R^{1b} is —NR¹¹CONR¹¹R¹²; R^{1a} is C₁-C₆ alkyl substituted with one or more —OSi(R¹³)₃, and R^{1b} is —CN; R^{1a} is C₁-C₆ alkyl substituted with one or more —OSi(R¹³)₃, and R^{1b} is —NR¹¹COR¹²; R^{1a} is C₁-C₆ alkyl substituted with one or more —OSi(R¹³)₃, and R^{1b} is —CR¹¹R¹²NR¹¹R¹²; R^{1a} is —SO₂NR¹¹R¹², and R^{1b} is C₁-C₆ alkyl substituted with one or more hydroxy; R^{1a} is —SO₂NR¹¹R¹², and R^{1b} is —SO₂NR¹¹R¹²; R^{1a} is —SO₂NR¹¹R¹², and R^{1b} is —SO₂R¹³; R^{1a} is —SO₂NR¹¹R¹², and R^{1b} is —CONR¹¹R¹²; R^{1a} is —SO₂NR¹¹R¹², and R^{1b} is —OR¹¹; R^{1a} is —SO₂NR¹¹R¹², and R^{1b} is —COR¹³; R^{1a} is —SO₂NR¹¹R¹², and R^{1b} is —CO₂R¹³; R^{1a} is —SO₂NR¹¹R¹², and R^{1b} is —NR¹³CONR¹¹R¹²; R^{1a} is —SO₂NR¹¹R¹², and R^{1b} is —CR¹¹R¹²CN; R^{1a} is —SO₂NR¹¹R¹², and R^{1b} is —NR¹¹SO₂R¹³; R^{1a} is —SO₂NR¹¹R¹², and R^{1b} is —CR¹¹R¹²NR¹¹R¹²; R^{1a} is —SO₂NR¹¹R¹², and R^{1b} is —CN; R^{1a} is —SO₂NR¹¹R¹², and R^{1b} is —NR¹¹CONR¹¹R¹²; R^{1a} is —SO₂NR¹¹R¹², and R^{1b} is —NR¹¹COR¹²; R^{1a} is —CR¹¹R¹²NR¹¹R¹², and R^{1b} is C₁-C₆ alkyl substituted with one or more hydroxy; R^{1a} is —CR¹¹R¹²NR¹¹R¹², and R^{1b} is —SO₂NR¹¹R¹²; R^{1a} is —CR¹¹R¹²NR¹¹R¹², and R^{1b} is —SO₂R¹³; R^{1a} is —CR¹¹R¹²NR¹¹R¹², and R^{1b} is —CONR¹¹R¹²; R^{1a} is —CR¹¹R¹²NR¹¹R¹², and R^{1b} is —OR¹¹; R^{1a} is —CR¹¹R¹²NR¹¹R¹², and R^{1b} is —COR¹³; R^{1a} is —CR¹¹R¹²NR¹¹R¹², and R^{1b} is —CO₂R¹³; R^{1a} is —CR¹¹R¹²NR¹¹R¹², and R^{1b} is —NR¹³CONR¹¹R¹²; R^{1a} is —CR¹¹R¹²NR¹¹R¹², and R^{1b} is —CR¹¹R¹²CN; R^{1a} is —CR¹¹R¹²NR¹¹R¹², and R^{1b} is —NR¹¹SO₂R¹³; R^{1a} is —CR¹¹R¹²NR¹¹R¹², and R^{1b} is —CR¹¹R¹²NR¹¹R¹²; R^{1a} is —CR¹¹R¹²NR¹¹R¹², and R^{1b} is —CN; R^{1a} is —CR¹¹R¹²NR¹¹R¹², and R^{1b} is —NR¹¹CONR¹¹R¹²; and R^{1a} is —CR¹¹R¹²NR¹¹R¹², and R^{1b} is —NR¹¹COR¹².

In some embodiments of the compound of formula AA,

the substituted ring A is

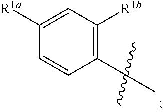

R^{1a} and R^{1b} are one of the following combinations:

In some embodiments, R^{1a} is 2-hydroxy-2-propyl, and R^{1b} is —OMe; In some embodiments, R^{1a} is 2-hydroxy-2-propyl, and R^{1b} is —OH; In some embodiments, R^{1a} is 2-hydroxy-2-propyl, and R^{1b} is —CO₂Me; In some embodiments, R^{1a} is 2-hydroxy-2-propyl, and R^{1b} is hydroxymethyl; In some embodiments, R^{1a} is 2-hydroxy-2-propyl, and R^{1b} is hydroxy ethyl; In some embodiments, R^{1a} is 2-hydroxy-2-propyl, and R^{1b} is 2-hydroxy-2-propyl; In some embodiments, R^{1a} is 2-hydroxy-2-propyl, and R^{1b} is —SO₂NHCH₂CH₂OH; In some embodiments, R^{1a} is 2-hydroxy-2-propyl, and R^{1b} is —SO₂Me; In some embodiments, R^{1a} is 2-hydroxy-2-propyl, and R^{1b} is CONHMe; In some embodiments, R^{1a} is 2-hydroxy-2-propyl, and R^{1b} is cyanomethyl; In some embodiments, R^{1a} is 2-hydroxy-2-propyl, and R^{1b} is —CN; In some embodiments, R^{1a} is 2-hydroxy-2-propyl, and R^{1b} is dimethylaminomethyl; In some embodiments, R^{1a} is dimethylaminomethyl, and R^{1b} is —OMe; In some embodiments, R^{1a} is dimethylaminomethyl, and R^{1b} is —OH; In some embodiments, R^{1a} is dimethylaminomethyl, and R^{1b} is —CO₂Me; In some embodiments, R^{1a} is dimethylaminomethyl, and R^{1b} is hydroxymethyl; In some embodiments, R^{1a} is dimethylaminomethyl, and R^{1b} is hydroxy ethyl; In some embodiments, R^{1a} is dimethylaminomethyl, and R^{1b} is 2-hydroxy-2-propyl; In some embodiments, R^{1a} is dimethylaminomethyl, and R^{1b} is —SO₂NHCH₂CH₂OH; In some embodiments, R^{1a} is dimethylaminomethyl, and R^{1b} is —SO₂Me; In some embodiments, R^{1a} is dimethylaminomethyl, and R^{1b} is CONHMe; In some embodiments, R^{1a} is dimethylaminomethyl, and R^{1b} is cyanomethyl; In some embodiments, R^{1a} is dimethylaminomethyl, and R^{1b} is dimethylaminomethyl; In some embodiments, R^{1a} is dimethylaminomethyl, and R^{1b} is CN; In some embodiments, R^{1a} is —SO₂NHMe, and R^{1b} is —OMe; In some embodiments, R^{1a} is —SO₂NHMe, and R^{1b} is —OH; In some embodiments, R^{1a} is —SO₂NHMe, and R^{1b} is —CO₂Me; In some embodiments, R^{1a} is —SO₂NHMe, and R^{1b} is hydroxymethyl; In some embodiments, R^{1a} is —SO₂NHMe, and R^{1b} is hydroxyethyl; In some embodiments, R^{1a} is —SO₂NHMe, and R^{1b} is 2-hydroxy-2-propyl; In some embodiments, R^{1a} is —SO₂NHMe, and R^{1b} is —SO₂NHCH₂CH₂OH; In some embodiments, R^{1a} is —SO₂NHMe, and R^{1b} is —SO₂Me; In some embodiments, R^{1a} is —SO₂NHMe, and R^{1b} is CONHMe; In some embodiments, R^{1a} is —SO₂NHMe, and R^{1b} is dimethylaminomethyl; In some embodiments, R^{1a} is —SO₂NHMe, and R^{1b} is —CN; In some embodiments, R^{1a} is —SO₂NHMe, and R^{1b} is cyanomethyl; and In some embodiments, R^{1a} is C₁-C₄ alkyl substituted with one —OSi(Me)₂tBu, and R^{1b} is —CO₂Me.

In some embodiments of the compound of formula AA,

the substituted ring A is

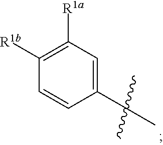

R^{1a} and R^{1b} are one of the following combinations:
R^{1a} is C₁-C₆ alkyl substituted with one or more hydroxy, and R^{1b} is C₁-C₆ alkyl substituted with one or more hydroxy; R^{1a} is C₁-C₆ alkyl substituted with one or more hydroxy, and R^{1b} is —SO₂NR¹¹R¹²; R^{1a} is C₁-C₆ alkyl substituted with one or more hydroxy, and R^{1b} is —SO₂R¹³; R^{1a} is C₁-C₆ alkyl substituted with one or more hydroxy, and R^{1b} is —CONR¹¹R¹²; R^{1a} is C₁-C₆ alkyl substituted with one or more hydroxy, and R^{1b} is —OR¹¹; R^{1a} is C₁-C₆ alkyl substituted with one or more hydroxy, and R^{1b} is —COR¹³; R^{1a} is C₁-C₆ alkyl substituted with one or more hydroxy, and R^{1b} is —CO₂R¹³; R^{1a} is C₁-C₆ alkyl substituted with one or more hydroxy, and R^{1b} is —NR¹³CONR¹¹R¹²; R^{1a} is C₁-C₆ alkyl substituted with one or more hydroxy, and R^{1b} is —CR¹¹R¹²CN; R^{1a} is C₁-C₆ alkyl substituted with one or more hydroxy, and R^{1b} is —NR¹¹SO₂R¹³; R^{1a} is C₁-C₆ alkyl substituted with one or more hydroxy, and R^{1b} is —NR¹¹CONR¹¹R¹²; R^{1a} is C₁-C₆ alkyl substituted with one or more hydroxy, and R^{1b} is —CN; R^{1a} is C₁-C₆ alkyl substituted with one or more hydroxy, and R^{1b} is —NR¹¹COR¹²; R^{1a} is C₁-C₆ alkyl substituted with one or more hydroxy, and R^{1b} is —CR¹¹R¹²NR¹¹R¹²; R^{1a} is C₁-C₆ alkyl substituted with one or more —OSi(R¹³)₃, and R^{1b} is C₁-C₆ alkyl substituted with one or more hydroxy; R^{1a} is C₁-C₆ alkyl substituted with one or more —OSi(R¹³)₃, and R^{1b} is —SO₂NR¹¹R¹²; R^{1a} is C₁-C₆ alkyl substituted with one or more —OSi(R¹³)₃, and R^{1b} is —SO₂R¹³; R^{1a} is C₁-C₆ alkyl substituted with one or more —OSi(R¹³)₃, and R^{1b} is —CONR¹¹R¹²; R^{1a} is C₁-C₆ alkyl substituted with one or more —OSi(R¹³)₃, and R^{1b} is —OR¹¹; R^{1a} is C₁-C₆ alkyl substituted with one or more —OSi(R¹³)₃, and R^{1b} is —COR¹³; R^{1a} is C₁-C₆ alkyl substituted with one or more —OSi(R¹³)₃, and R^{1b} is —CO₂R¹³; R^{1a} is C₁-C₆ alkyl substituted with one or more —OSi(R¹³)₃, and R^{1b} is —NR¹³CONR¹¹R¹²; R^{1a} is C₁-C₆ alkyl substituted with one or more —OSi(R¹³)₃, and R^{1b} is —CR¹¹R¹²CN; R^{1a} is C₁-C₆ alkyl substituted with one or more —OSi(R¹³)₃, and R^{1b} is —NR¹¹SO₂R¹³; R^{1a} is C₁-C₆ alkyl substituted with one or more —OSi(R¹³)₃, and R^{1b} is —NR¹¹CONR¹¹R¹²; R^{1a} is C₁-C₆ alkyl substituted with one or more —OSi(R¹³)₃, and R^{1b} is —CN; R^{1a} is C₁-C₆ alkyl substituted with one or more —OSi(R¹³)₃, and R^{1b} is —NR¹¹COR¹²; R^{1a} is C₁-C₆ alkyl substituted with one or more —OSi(R¹³)₃, and R^{1b} is —CR¹¹R¹²NR¹¹R¹²; R^{1a} is —SO₂NR¹¹R¹², and R^{1b} is C₁-C₆ alkyl substituted with one or more hydroxy; R^{1a} is —SO₂NR¹¹R¹², and R^{1b} is —SO₂NR¹¹R¹²; R^{1a} is —SO₂NR¹¹R¹², and R^{1b} is —SO₂R¹³; R^{1a} is —SO₂NR¹¹R¹², and R^{1b} is —CONR¹¹R¹²; R^{1a} is —SO₂NR¹¹R¹², and R^{1b} is —OR¹¹; R^{1a} is —SO₂NR¹¹R¹², and R^{1b} is —COR¹³; R^{1a} is —SO₂NR¹¹R¹², and R^{1b} is —CO₂R¹³; R^{1a} is —SO₂NR¹¹R¹², and R^{1b} is —NR¹³CONR¹¹R¹²; R^{1a} is —SO₂NR¹¹R¹², and R^{1b} is —CR¹¹R¹²CN; R^{1a} is —SO₂NR¹¹R¹², and R^{1b} is —NR¹¹SO₂R¹³; R^{1a} is —SO₂NR¹¹R¹², and R^{1b} is —CR¹¹R¹²NR¹¹R¹²; R^{1a} is —SO₂NR¹¹R¹², and R^{1b} is —CN; R^{1a} is —SO₂NR¹¹R¹², and R^{1b} is —NR¹¹CONR¹¹R¹²; R^{1a} is —SO₂NR¹¹R¹², and R^{1b} is —NR¹¹COR¹²; R^{1a} is —CR¹¹R¹²NR¹¹R¹², and R^{1b} is C₁-C₆ alkyl substituted with one or more hydroxy; R^{1a} is —CR¹¹R¹²NR¹¹R¹², and R^{1b} is —SO₂NR¹¹R¹²; R^{1a} is —CR¹¹R¹²NR¹¹R¹², and R^{1b} is —SO₂R¹³; R^{1a} is —CR¹¹R¹²NR¹¹R¹², and R^{1b} is —CONR¹¹R¹²; R^{1a} is —CR¹¹R¹²NR¹¹R¹², and R^{1b} is —OR¹¹; R^{1a} is —CR¹¹R¹²NR¹¹R¹², and R^{1b} is —COR¹³; R^{1a} is —CR¹¹R¹²NR¹¹R¹², and R^{1b} is —CO₂R¹³; R^{1a} is —CR¹¹R¹²NR¹¹R¹², and R^{1b} is —NR¹³CONR¹¹R¹²; R^{1a} is —CR¹¹R¹²NR¹¹R¹², and R^{1b} is —CR¹¹R¹²CN; R^{1a} is —CR¹¹R¹²NR¹¹R¹², and R^{1b} is —NR¹¹SO₂R¹³; R^{1a} is —CR¹¹R¹²NR¹¹R¹², and R^{1b} is —CR¹¹R¹²NR¹¹R¹²; R^{1a} is —CR¹¹R¹²NR¹¹R¹², and R^{1b} is —CN; R^{1a} is —CR¹¹R¹²NR¹¹R¹², and R^{1b} is —NR¹¹CONR¹¹R¹²; and R^{1a} is —CR¹¹R¹²NR¹¹R¹², and R^{1b} is —NR¹¹COR¹².

In some embodiments of the compound of formula AA,

the substituted ring A is

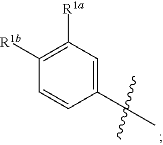

R^{1a} and R^{1b} are one of the following combinations:

In some embodiments, R^{1a} is 2-hydroxy-2-propyl, and R^{1b} is —OMe; In some embodiments, R^{1a} is 2-hydroxy-2-propyl, and R^{1b} is —OH; In some embodiments, R^{1a} is 2-hydroxy-2-propyl, and R^{1b} is —CO₂Me; In some embodiments, R^{1a} is 2-hydroxy-2-propyl, and R^{1b} is hydroxymethyl; In some embodiments, R^{1a} is 2-hydroxy-2-propyl, and R^{1b} is hydroxy ethyl; In some embodiments, R^{1a} is 2-hydroxy-2-propyl, and R^{1b} is 2-hydroxy-2-propyl; In some embodiments, R^{1a} is 2-hydroxy-2-propyl, and R^{1b} is —SO₂NHCH₂CH₂OH; In some embodiments, R^{1a} is 2-hydroxy-2-propyl, and R^{1b} is —SO₂Me; In some embodiments, R^{1a} is 2-hydroxy-2-propyl, and R^{1b} is CONHMe; In some embodiments, R^{1a} is 2-hydroxy-2-propyl, and R^{1b} is cyanomethyl; In some embodiments, R^{1a} is 2-hydroxy-2-propyl, and R^{1b} is —CN; In some embodiments, R^{1a} is 2-hydroxy-2-propyl, and R^{1b} is dimethylaminomethyl; In some embodiments, R^{1a} is dimethylaminomethyl, and R^{1b} is —OMe; In some embodiments, R^{1a} is dimethylaminomethyl, and R^{1b} is —OH; In some embodiments, R^{1a} is dimethylaminomethyl, and R^{1b} is —CO₂Me; In some embodiments, R^{1a} is dimethylaminomethyl, and R^{1b} is hydroxymethyl; In some embodiments, R^{1a} is dimethylaminomethyl, and R^{1b} is hydroxy ethyl; In some embodiments, R^{1a} is dimethylaminomethyl, and R^{1b} is 2-hydroxy-2-propyl; In some embodiments, R^{1a} is dimethylaminomethyl, and R^{1b} is —SO₂NHCH₂CH₂OH; In some embodiments, R^{1a} is dimethylaminomethyl, and R^{1b} is —SO₂Me; In some embodiments, R^{1a} is dimethylaminomethyl, and R^{1b} is CONHMe; In some embodiments, R^{1a} is dimethylaminomethyl, and R^{1b} is cyanomethyl; In some embodiments, R^{1a} is dimethylaminomethyl, and R^{1b} is dimethylaminomethyl; In some embodiments, R^{1a} is dimethylaminomethyl, and R^{1b} is CN; In some embodiments, R^{1a} is —SO₂NHMe, and R^{1b} is —OMe; In some embodiments, R^{1a} is —SO₂NHMe, and R^{1b} is —OH; In some embodiments, R^{1a} is —SO₂NHMe, and R^{1b} is —CO₂Me; In some embodiments, R^{1a} is —SO₂NHMe, and R^{1b} is hydroxymethyl; In some embodiments, R^{1a} is —SO₂NHMe, and R^{1b} is hydroxyethyl; In some embodiments, R^{1a} is —SO₂NHMe, and R^{1b} is 2-hydroxy-2-propyl; In some embodiments, R^{1a} is —SO₂NHMe, and R^{1b} is —SO₂NHCH₂CH₂OH; In some embodiments, R^{1a} is —SO₂NHMe, and R^{1b} is —SO₂Me; In some embodiments, R^{1a} is —SO₂NHMe, and R^{1b} is CONHMe; In some embodiments, R^{1a} is —SO₂NHMe, and R^{1b} is dimethylaminomethyl; In some embodiments, R^{1a} is —SO₂NHMe, and R^{1b} is —CN; In some embodiments, R^{1a} is —SO₂NHMe, and R^{1b} is cyanomethyl; and In some embodiments, R^{1a} is C₁-C₄ alkyl substituted with one —OSi(Me)₂tBu, and R^{1b} is —CO₂Me.

In some embodiments of the compound of formula AA,

the substituted ring A is

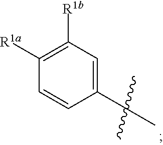

R^{1a} and R^{1b} are one of the following combinations:

In some embodiments, R^{1a} is 2-hydroxy-2-propyl, and R^{1b} is —OMe; In some embodiments, R^{1a} is 2-hydroxy-2-propyl, and R^{1b} is —OH; In some embodiments, R^{1a} is 2-hydroxy-2-propyl, and R^{1b} is —CO₂Me; In some embodiments, R^{1a} is 2-hydroxy-2-propyl, and R^{1b} is hydroxymethyl; In some embodiments, R^{1a} is 2-hydroxy-2-propyl, and R^{1b} is hydroxy ethyl; In some embodiments, R^{1a} is 2-hydroxy-2-propyl, and R^{1b} is 2-hydroxy-2-propyl; In some embodiments, R^{1a} is 2-hydroxy-2-propyl, and R^{1b} is —SO₂NHCH₂CH₂OH; In some embodiments, R^{1a} is 2-hydroxy-2-propyl, and R^{1b} is —SO₂Me; In some embodiments, R^{1a} is 2-hydroxy-2-propyl, and R^{1b} is CONHMe; In some embodiments, R^{1a} is 2-hydroxy-2-propyl, and R^{1b} is cyanomethyl; In some embodiments, R^{1a} is 2-hydroxy-2-propyl, and R^{1b} is —CN; In some embodiments, R^{1a} is 2-hydroxy-2-propyl, and R^{1b} is dimethylaminomethyl; In some embodiments, R^{1a} is dimethylaminomethyl, and R^{1b} is —OMe; In some embodiments, R^{1a} is dimethylaminomethyl, and R^{1b} is —OH; In some embodiments, R^{1a} is dimethylaminomethyl, and R^{1b} is —CO₂Me; In some embodiments, R^{1a} is dimethylaminomethyl, and R^{1b} is hydroxymethyl; In some embodiments, R^{1a} is dimethylaminomethyl, and R^{1b} is hydroxy ethyl; In some embodiments, R^{1a} is dimethylaminomethyl, and R^{1b} is 2-hydroxy-2-propyl; In some embodiments, R^{1a} is dimethylaminomethyl, and R^{1b} is —SO₂NHCH₂CH₂OH; In some embodiments, R^{1a} is dimethylaminomethyl, and R^{1b} is —SO₂Me; In some embodiments, R^{1a} is dimethylaminomethyl, and R^{1b} is CONHMe; In some embodiments, R^{1a} is dimethylaminomethyl, and R^{1b} is cyanomethyl; In some embodiments, R^{1a} is dimethylaminomethyl, and R^{1b} is dimethylaminomethyl; In some embodiments, R^{1a} is dimethylaminomethyl, and R^{1b} is CN; In some embodiments, R^{1a} is —SO₂NHMe, and R^{1b} is —OMe; In some embodiments, R^{1a} is —SO₂NHMe, and R^{1b} is —OH; In some embodiments, R^{1a} is —SO₂NHMe, and R^{1b} is —CO₂Me; In some embodiments, R^{1a} is —SO₂NHMe, and R^{1b} is hydroxymethyl; In some embodiments, R^{1a} is —SO₂NHMe, and R^{1b} is hydroxyethyl; In some embodiments, R^{1a} is —SO₂NHMe, and R^{1b} is 2-hydroxy-2-propyl; In some embodiments, R^{1a} is —SO₂NHMe, and R^{1b} is —SO₂NHCH₂CH₂OH; In some embodiments, R^{1a} is —SO₂NHMe, and R^{1b} is —SO₂Me; In some embodiments, R^{1a} is —SO₂NHMe, and R^{1b} is CONHMe; In some embodiments, R^{1a} is —SO₂NHMe, and R^{1b} is dimethylaminomethyl; In some embodiments, R^{1a} is —SO₂NHMe, and R^{1b} is —CN; In some embodiments, R^{1a} is —SO₂NHMe, and R^{1b} is cyanomethyl; and In some embodiments, R^{1a} is C₁-C₄ alkyl substituted with one —OSi(Me)₂tBu, and R^{1b} is —CO₂Me.

In some embodiments of the compound of formula AA,

the substituted ring A is

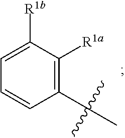

R^{1a} and R^{1b} are one of the following combinations:
R^{1a} is C₁-C₆ alkyl substituted with one or more hydroxy, and R^{1b} is C₁-C₆ alkyl substituted with one or more hydroxy; R^{1a} is C₁-C₆ alkyl substituted with one or more hydroxy, and R^{1b} is —SO₂NR¹¹R¹²; R^{1a} is C₁-C₆ alkyl substituted with one or more hydroxy, and R^{1b} is —SO₂R¹³; R^{1a} is C₁-C₆ alkyl substituted with one or more hydroxy, and R^{1b} is —CONR¹¹R¹²; R^{1a} is C₁-C₆ alkyl substituted with one or more hydroxy, and R^{1b} is —OR¹¹; R^{1a} is C₁-C₆ alkyl substituted with one or more hydroxy, and R^{1b} is —COR¹³; R^{1a} is C₁-C₆ alkyl substituted with one or more hydroxy, and R^{1b} is —CO₂R¹³; R^{1a} is C₁-C₆ alkyl substituted with one or more hydroxy, and R^{1b} is —NR¹³CONR¹¹R¹²; R^{1a} is C₁-C₆ alkyl substituted with one or more hydroxy, and R^{1b} is —CR¹¹R¹²CN; R^{1a} is C₁-C₆ alkyl substituted with one or more hydroxy, and R^{1b} is —NR¹¹SO₂R¹³; R^{1a} is C₁-C₆ alkyl substituted with one or more hydroxy, and R^{1b} is —NR¹¹CONR¹¹R¹²; R^{1a} is C₁-C₆ alkyl substituted with one or more hydroxy, and R^{1b} is —CN; R^{1a} is C₁-C₆ alkyl substituted with one or more hydroxy, and R^{1b} is —NR¹¹COR¹²; R^{1a} is C₁-C₆ alkyl substituted with one or more hydroxy, and R^{1b} is —CR¹¹R¹²NR¹¹R¹²; R^{1a} is C₁-C₆ alkyl substituted with one or more —OSi(R¹³)₃, and R^{1b} is C₁-C₆ alkyl substituted with one or more hydroxy; R^{1a} is C₁-C₆ alkyl substituted with one or more —OSi(R¹³)₃, and R^{1b} is —SO₂NR¹¹R¹²; R^{1a} is C₁-C₆ alkyl substituted with one or more —OSi(R¹³)₃, and R^{1b} is —SO₂R¹³; R^{1a} is C₁-C₆ alkyl substituted with one or more —OSi(R¹³)₃, and R^{1b} is —CONR¹¹R¹²; R^{1a} is C₁-C₆ alkyl substituted with one or more —OSi(R¹³)₃, and R^{1b} is —OR¹¹; R^{1a} is C₁-C₆ alkyl substituted with one or more —OSi(R¹³)₃, and R^{1b} is —COR¹³; R^{1a} is C₁-C₆ alkyl substituted with one or more —OSi(R¹³)₃, and R^{1b} is —CO₂R¹³; R^{1a} is C₁-C₆ alkyl substituted with one or more —OSi(R¹³)₃, and R^{1b} is —NR¹³CONR¹¹R¹²; R^{1a} is C₁-C₆ alkyl substituted with one or more —OSi(R¹³)₃, and R^{1b} is —CR¹¹R¹²CN; R^{1a} is C₁-C₆ alkyl substituted with one or more —OSi(R¹³)₃, and R^{1b} is —NR¹¹SO₂R¹³; R^{1a} is C₁-C₆ alkyl substituted with one or more —OSi(R¹³)₃, and R^{1b} is —NR¹¹CONR¹¹R¹²; R^{1a} is C₁-C₆ alkyl substituted with one or more —OSi(R¹³)₃, and R^{1b} is —CN; R^{1a} is C₁-C₆ alkyl substituted with one or more —OSi(R¹³)₃, and R^{1b} is —NR¹¹COR¹²; R^{1a} is C₁-C₆ alkyl substituted with one or more —OSi(R¹³)₃, and R^{1b} is —CR¹¹R¹²NR¹¹R¹²; R^{1a} is —SO₂NR¹¹R¹², and R^{1b} is C₁-C₆ alkyl substituted with one or more hydroxy; R^{1a} is —SO₂NR¹¹R¹², and R^{1b} is —SO₂NR¹¹R¹²; R^{1a} is —SO₂NR¹¹R¹², and R^{1b} is —SO₂R¹³; R^{1a} is —SO₂NR¹¹R¹², and R^{1b} is —CONR¹¹R¹²; R^{1a} is —SO₂NR¹¹R¹², and R^{1b} is —OR¹¹; R^{1a} is —SO₂NR¹¹R¹², and R^{1b} is —COR¹³; R^{1a} is —SO₂NR¹¹R¹², and R^{1b} is —CO₂R¹³; R^{1a} is —SO₂NR¹¹R¹², and R^{1b} is —NR¹³CONR¹¹R¹²; R^{1a} is —SO₂NR¹¹R¹², and R^{1b} is —CR¹¹R¹²CN; R^{1a} is —SO₂NR¹¹R¹², and R^{1b} is —NR¹¹SO₂R¹³; R^{1a} is —SO₂NR¹¹R¹², and R^{1b} is —CR¹¹R¹²NR¹¹R¹²; R^{1a} is —SO₂NR¹¹R¹², and R^{1b} is —CN; R^{1a} is —SO₂NR¹¹R¹², and R^{1b} is —NR¹¹CONR¹¹R¹²; R^{1a} is —SO₂NR¹¹R¹², and R^{1b} is —NR¹¹COR¹²; R^{1a} is —CR¹¹R¹²NR¹¹R¹², and R^{1b} is C₁-C₆ alkyl substituted with one or more hydroxy; R^{1a} is —CR¹¹R¹²NR¹¹R¹², and R^{1b} is —SO₂NR¹¹R¹²; R^{1a} is —CR¹¹R¹²NR¹¹R¹², and R^{1b} is —SO₂R¹³; R^{1a} is —CR¹¹R¹²NR¹¹R¹², and R^{1b} is —CONR¹¹R¹²; R^{1a} is —CR¹¹R¹²NR¹¹R¹², and R^{1b} is —OR¹¹; R^{1a} is —CR¹¹R¹²NR¹¹R¹², and R^{1b} is —COR¹³; R^{1a} is —CR¹¹R¹²NR¹¹R¹², and R^{1b} is —CO₂R¹³; R^{1a} is —CR¹¹R¹²NR¹¹R¹², and R^{1b} is —NR¹³CONR¹¹R¹²; R^{1a} is —CR¹¹R¹²NR¹¹R¹², and R^{1b} is —CR¹¹R¹²CN; R^{1a} is —CR¹¹R¹²NR¹¹R¹², and R^{1b} is —NR¹¹SO₂R¹³; R^{1a} is —CR¹¹R¹²NR¹¹R¹², and R^{1b} is —CR¹¹R¹²NR¹¹R¹²; R^{1a} is —CR¹¹R¹²NR¹¹R¹², and R^{1b} is —CN; R^{1a} is —CR¹¹R¹²NR¹¹R¹², and R^{1b} is —NR¹¹CONR¹¹R¹²; and R^{1a} is —CR¹¹R¹²NR¹¹R¹², and R^{1b} is —NR¹¹COR¹².

In some embodiments of the compound of formula AA,

the substituted ring A is

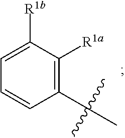

R^{1a} and R^{1b} are one of the following combinations:

In some embodiments, R^{1a} is 2-hydroxy-2-propyl, and R^{1b} is —OMe; In some embodiments, R^{1a} is 2-hydroxy-2-propyl, and R^{1b} is —OH; In some embodiments, R^{1a} is 2-hydroxy-2-propyl, and R^{1b} is —CO₂Me; In some embodiments, R^{1a} is 2-hydroxy-2-propyl, and R^{1b} is hydroxymethyl; In some embodiments, R^{1a} is 2-hydroxy-2-propyl, and R^{1b} is hydroxy ethyl; In some embodiments, R^{1a} is 2-hydroxy-2-propyl, and R^{1b} is 2-hydroxy-2-propyl; In some embodiments, R^{1a} is 2-hydroxy-2-propyl, and R^{1b} is —SO₂NHCH₂CH₂OH; In some embodiments, R^{1a} is 2-hydroxy-2-propyl, and R^{1b} is —SO₂Me; In some embodiments, R^{1a} is 2-hydroxy-2-propyl, and R^{1b} is CONHMe; In some embodiments, R^{1a} is 2-hydroxy-2-propyl, and R^{1b} is cyanomethyl; In some embodiments, R^{1a} is 2-hydroxy-2-propyl, and R^{1b} is —CN; In some embodiments, R^{1a} is 2-hydroxy-2-propyl, and R^{1b} is dimethylaminomethyl; In some embodiments, R^{1a} is dimethylaminomethyl, and R^{1b} is —OMe; In some embodiments, R^{1a} is dimethylaminomethyl, and R^{1b} is —OH; In some embodiments, R^{1a} is dimethylaminomethyl, and R^{1b} is —CO₂Me; In some embodiments, R^{1a} is dimethylaminomethyl, and R^{1b} is hydroxymethyl; In some embodiments, R^{1a} is dimethylaminomethyl, and R^{1b} is hydroxy ethyl; In some embodiments, R^{1a} is dimethylaminomethyl, and R^{1b} is 2-hydroxy-2-propyl; In some embodiments, R^{1a} is dimethylaminomethyl, and R^{1b} is —SO₂NHCH₂CH₂OH; In some embodiments, R^{1a} is dimethylaminomethyl, and R^{1b} is —SO₂Me; In some embodiments, R^{1a} is dimethylaminomethyl, and R^{1b} is CONHMe; In some embodiments, R^{1a} is dimethylaminomethyl, and R^{1b} is cyanomethyl; In some embodiments, R^{1a} is dimethylaminomethyl, and R^{1b} is dimethylaminomethyl; In some embodiments, R^{1a} is dimethylaminomethyl, and R^{1b} is CN; In some embodiments, R^{1a} is —SO₂NHMe, and R^{1b} is —OMe; In some embodiments, R^{1a} is —SO₂NHMe, and R^{1b} is —OH; In some embodiments, R^{1a} is —SO₂NHMe, and R^{1b} is —CO₂Me; In some embodiments, R^{1a} is —SO₂NHMe, and R^{1b} is hydroxymethyl; In some embodiments, R^{1a} is —SO₂NHMe, and R^{1b} is hydroxyethyl; In some embodiments, R^{1a} is —SO₂NHMe, and R^{1b} is 2-hydroxy-2-propyl; In some embodiments, R^{1a} is —SO₂NHMe, and R^{1b} is —SO₂NHCH₂CH₂OH; In some embodiments, R^{1a} is —SO₂NHMe, and R^{1b} is —SO₂Me; In some embodiments, R^{1a} is —SO₂NHMe, and R^{1b} is CONHMe; In some embodiments, R^{1a} is —SO₂NHMe, and R^{1b} is dimethylaminomethyl; In some embodiments, R^{1a} is —SO₂NHMe, and R^{1b} is —CN; In some embodiments, R^{1a} is —SO₂NHMe, and R^{1b} is cyanomethyl; and In some embodiments, R^{1a} is Q-C₄ alkyl substituted with one —OSi(Me)₂tBu, and R^{1b} is —CO₂Me.

In some embodiments of the compound of formula AA,

the substituted ring A is

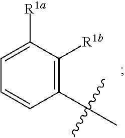

R^{1a} and R^{1b} are one of the following combinations:
R^{1a} is C₁-C₆ alkyl substituted with one or more hydroxy, and R^{1b} is C₁-C₆ alkyl substituted with one or more hydroxy; R^{1a} is C₁-C₆ alkyl substituted with one or more hydroxy, and R^{1b} is —SO₂NR¹¹R¹²; R^{1a} is C₁-C₆ alkyl substituted with one or more hydroxy, and R^{1b} is —SO₂R¹³; R^{1a} is C₁-C₆ alkyl substituted with one or more hydroxy, and R^{1b} is —CONR¹¹R¹²; R^{1a} is C₁-C₆ alkyl substituted with one or more hydroxy, and R^{1b} is —OR¹¹; R^{1a} is C₁-C₆ alkyl substituted with one or more hydroxy, and R^{1b} is —COR¹³; R^{1a} is C₁-C₆ alkyl substituted with one or more hydroxy, and R^{1b} is —CO₂R¹³; R^{1a} is C₁-C₆ alkyl substituted with one or more hydroxy, and R^{1b} is —NR¹³CONR¹¹R¹²; R^{1a} is C₁-C₆ alkyl substituted with one or more hydroxy, and R^{1b} is —CR¹¹R¹²CN; R^{1a} is C₁-C₆ alkyl substituted with one or more hydroxy, and R^{1b} is —NR¹¹SO₂R¹³; R^{1a} is C₁-C₆ alkyl substituted with one or more hydroxy, and R^{1b} is —NR¹¹CONR¹¹R¹²; R^{1a} is C₁-C₆ alkyl substituted with one or more hydroxy, and R^{1b} is —CN; R^{1a} is C₁-C₆ alkyl substituted with one or more hydroxy, and R^{1b} is —NR¹¹COR¹²; R^{1a} is C₁-C₆ alkyl substituted with one or more hydroxy, and R^{1b} is —CR¹¹R¹²NR¹¹R¹²; R^{1a} is C₁-C₆ alkyl substituted with one or more —OSi(R¹³)₃, and R^{1b} is C₁-C₆ alkyl substituted with one or more hydroxy; R^{1a} is C₁-C₆ alkyl substituted with one or more —OSi(R¹³)₃, and R^{1b} is —SO₂NR¹¹R¹²; R^{1a} is C₁-C₆ alkyl substituted with one or more —OSi(R¹³)₃, and R^{1b} is —SO₂R¹³; R^{1a} is C₁-C₆ alkyl substituted with one or more —OSi(R¹³)₃, and R^{1b} is —CONR¹¹R¹²; R^{1a} is C₁-C₆ alkyl substituted with one or more —OSi(R¹³)₃, and R^{1b} is —OR¹¹; R^{1a} is C₁-C₆ alkyl substituted with one or more —OSi(R¹³)₃, and R^{1b} is —COR¹³; R^{1a} is C₁-C₆ alkyl substituted with one or more —OSi(R¹³)₃, and R^{1b} is —CO₂R¹³; R^{1a} is C₁-C₆ alkyl substituted with one or more —OSi(R¹³)₃, and R^{1b} is —NR¹³CONR¹¹R¹²; R^{1a} is C₁-C₆ alkyl substituted with one or more —OSi(R¹³)₃, and R^{1b} is —CR¹¹R¹²CN; R^{1a} is C₁-C₆ alkyl substituted with one or more —OSi(R¹³)₃, and R^{1b} is —NR¹¹SO₂R¹³; R^{1a} is C₁-C₆ alkyl substituted with one or more —OSi(R¹³)₃, and R^{1b} is —NR¹¹CONR¹¹R¹²; R^{1a} is C₁-C₆ alkyl substituted with one or more —OSi(R¹³)₃, and R^{1b} is —CN; R^{1a} is C₁-C₆ alkyl substituted with one or more —OSi(R¹³)₃, and R^{1b} is —NR¹¹COR¹²; R^{1a} is C₁-C₆ alkyl substituted with one or more —OSi(R¹³)₃, and R^{1b} is —CR¹¹R¹²NR¹¹R¹²; R^{1a} is —SO₂NR¹¹R¹², and R^{1b} is C₁-C₆ alkyl substituted with one or more hydroxy; R^{1a} is —SO₂NR¹¹R¹², and R^{1b} is —SO₂NR¹¹R¹²; R^{1a} is —SO₂NR¹¹R¹², and R^{1b} is —SO₂R¹³; R^{1a} is —SO₂NR¹¹R¹², and R^{1b} is —CONR¹¹R¹²; R^{1a} is —SO₂NR¹¹R¹², and R^{1b} is —OR¹¹; R^{1a} is —SO₂NR¹¹R¹², and R^{1b} is —COR¹³; R^{1a} is —SO₂NR¹¹R¹², and R^{1b} is —CO₂R¹³; R^{1a} is —SO₂NR¹¹R¹², and R^{1b} is —NR¹³CONR¹¹R¹²; R^{1a} is —SO₂NR¹¹R¹², and R^{1b} is —CR¹¹R¹²CN; R^{1a} is —SO₂NR¹¹R¹², and R^{1b} is —NR¹¹SO₂R¹³; R^{1a} is —SO₂NR¹¹R¹², and R^{1b} is —CR¹¹R¹²NR¹¹R¹²; R^{1a} is —SO₂NR¹¹R¹², and R^{1b} is —CN; R^{1a} is —SO₂NR¹¹R¹², and R^{1b} is —NR¹¹CONR¹¹R¹²; R^{1a} is —SO₂NR¹¹R¹², and R^{1b} is —NR¹¹COR¹²; R^{1a} is —CR¹¹R¹²NR¹¹R¹², and R^{1b} is C₁-C₆ alkyl substituted with one or more hydroxy; R^{1a} is —CR¹¹R¹²NR¹¹R¹², and R^{1b} is —SO₂NR¹¹R¹²; R^{1a} is —CR¹¹R¹²NR¹¹R¹², and R^{1b} is —SO₂R¹³; R^{1a} is —CR¹¹R¹²NR¹¹R¹², and R^{1b} is —CONR¹¹R¹²; R^{1a} is —CR¹¹R¹²NR¹¹R¹², and R^{1b} is —OR¹¹; R^{1a} is —CR¹¹R¹²NR¹¹R¹², and R^{1b} is —COR¹³; R^{1a} is —CR¹¹R¹²NR¹¹R¹², and R^{1b} is —CO₂R¹³; R^{1a} is —CR¹¹R¹²NR¹¹R¹², and R^{1b} is —NR¹³CONR¹¹R¹²; R^{1a} is —CR¹¹R¹²NR¹¹R¹², and R^{1b} is —CR¹¹R¹²CN; R^{1a} is —CR¹¹R¹²NR¹¹R¹², and R^{1b} is —NR¹¹SO₂R¹³; R^{1a} is —CR¹¹R¹²NR¹¹R¹², and R^{1b} is —CR¹¹R¹²NR¹¹R¹²; R^{1a} is —CR¹¹R¹²NR¹¹R¹², and R^{1b} is —CN; R^{1a} is —CR¹¹R¹²NR¹¹R¹², and R^{1b} is —NR¹¹CONR¹¹R¹²; and R^{1a} is —CR¹¹R¹²NR¹¹R¹², and R^{1b} is —NR¹¹COR¹².

In some embodiments of the compound of formula AA,

the substituted ring A is

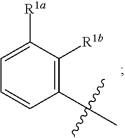

R^{1a} and R^{1b} are one of the following combinations:

In some embodiments, R^{1a} is 2-hydroxy-2-propyl, and R^{1b} is —OMe; In some embodiments, R^{1a} is 2-hydroxy-2-propyl, and R^{1b} is —OH; In some embodiments, R^{1a} is 2-hydroxy-2-propyl, and R^{1b} is —CO₂Me; In some embodiments, R^{1a} is 2-hydroxy-2-propyl, and R^{1b} is hydroxymethyl; In some embodiments, R^{1a} is 2-hydroxy-2-propyl, and R^{1b} is hydroxy ethyl; In some embodiments, R^{1a} is 2-hydroxy-2-propyl, and R^{1b} is 2-hydroxy-2-propyl; In some embodiments, R^{1a} is 2-hydroxy-2-propyl, and R^{1b} is —SO₂NHCH₂CH₂OH; In some embodiments, R^{1a} is 2-hydroxy-2-propyl, and R^{1b} is —SO₂Me; In some embodiments, R^{1a} is 2-hydroxy-2-propyl, and R^{1b} is CONHMe; In some embodiments, R^{1a} is 2-hydroxy-2-propyl, and R^{1b} is cyanomethyl; In some embodiments, R^{1a} is 2-hydroxy-2-propyl, and R^{1b} is —CN; In some embodiments, R^{1a} is 2-hydroxy-2-propyl, and R^{1b} is dimethylaminomethyl; In some embodiments, R^{1a} is dimethylaminomethyl, and R^{1b} is —OMe; In some embodiments, R^{1a} is dimethylaminomethyl, and R^{1b} is —OH; In some embodiments, R^{1a} is dimethylaminomethyl, and R^{1b} is —CO₂Me; In some embodiments, R^{1a} is dimethylaminomethyl, and R^{1b} is hydroxymethyl; In some embodiments, R^{1a} is dimethylaminomethyl, and R^{1b} is hydroxy ethyl; In some embodiments, R^{1a} is dimethylaminomethyl, and R^{1b} is 2-hydroxy-2-propyl; In some embodiments, R^{1a} is dimethylaminomethyl, and R^{1b} is —SO₂NHCH₂CH₂OH; In some embodiments, R^{1a} is dimethylaminomethyl, and R^{1b} is —SO₂Me; In some embodiments, R^{1a} is dimethylaminomethyl, and R^{1b} is CONHMe; In some embodiments, R^{1a} is dimethylaminomethyl, and R^{1b} is cyanomethyl; In some embodiments, R^{1a} is dimethylaminomethyl, and R^{1b} is dimethylaminomethyl; In some embodiments, R^{1a} is dimethylaminomethyl, and R^{1b} is CN; In some embodiments, R^{1a} is —SO₂NHMe, and R^{1b} is —OMe; In some embodiments, R^{1a} is —SO₂NHMe, and R^{1b} is —OH; In some embodiments, R^{1a} is —SO₂NHMe, and R^{1b} is —CO₂Me; In some embodiments, R^{1a} is —SO₂NHMe, and R^{1b} is hydroxymethyl; In some embodiments, R^{1a} is —SO₂NHMe, and R^{1b} is hydroxyethyl; In some embodiments, R^{1a} is —SO₂NHMe, and R^{1b} is 2-hydroxy-2-propyl; In some embodiments, R^{1a} is —SO₂NHMe, and R^{1b} is —SO₂NHCH₂CH₂OH; In some embodiments, R^{1a} is —SO₂NHMe, and R^{1b} is —SO₂Me; In some embodiments, R^{1a} is —SO₂NHMe, and R^{1b} is CONHMe; In some embodiments, R^{1a} is —SO₂NHMe, and R^{1b} is dimethylaminomethyl; In some embodiments, R^{1a} is —SO₂NHMe, and R^{1b} is —CN; In some embodiments, R^{1a} is —SO₂NHMe, and R^{1b} is cyanomethyl; and In some embodiments, R^{1a} is C₁-C₄ alkyl substituted with one —OSi(Me)₂tBu, and R^{1b} is —CO₂Me.

In some embodiments of the compound of formula AA,

the substituted ring A is

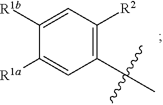

R^{1a} and R^{1b} are one of the following combinations:
R^{1a} is C₁-C₆ alkyl substituted with one or more hydroxy, and R^{1b} is C₁-C₆ alkyl substituted with one or more hydroxy; R^{1a} is C₁-C₆ alkyl substituted with one or more hydroxy, and R^{1b} is —SO₂NR¹¹R¹²; R^{1a} is C₁-C₆ alkyl substituted with one or more hydroxy, and R^{1b} is —SO₂R¹³; R^{1a} is C₁-C₆ alkyl substituted with one or more hydroxy, and R^{1b} is —CONR¹¹R¹²; R^{1a} is C₁-C₆ alkyl substituted with one or more hydroxy, and R^{1b} is —OR¹¹; R^{1a} is C₁-C₆ alkyl substituted with one or more hydroxy, and R^{1b} is —COR¹³; R^{1a} is C₁-C₆ alkyl substituted with one or more hydroxy, and R^{1b} is —CO₂R¹³; R^{1a} is C₁-C₆ alkyl substituted with one or more hydroxy, and R^{1b} is —NR¹³CONR¹¹R¹²; R^{1a} is C₁-C₆ alkyl substituted with one or more hydroxy, and R^{1b} is —CR¹¹R¹²CN; R^{1a} is C₁-C₆ alkyl substituted with one or more hydroxy, and R^{1b} is —NR¹¹SO₂R¹³; R^{1a} is C₁-C₆ alkyl substituted with one or more hydroxy, and R^{1b} is —NR¹¹CONR¹¹R¹²; R^{1a} is C₁-C₆ alkyl substituted with one or more hydroxy, and R^{1b} is —CN; R^{1a} is C₁-C₆ alkyl substituted with one or more hydroxy, and R^{1b} is —NR¹¹COR¹²; R^{1a} is C₁-C₆ alkyl substituted with one or more hydroxy, and R^{1b} is —CR¹¹R¹²NR¹¹R¹²; R^{1a} is C₁-C₆ alkyl substituted with one or more —OSi(R¹³)₃, and R^{1b} is C₁-C₆ alkyl substituted with one or more hydroxy; R^{1a} is C₁-C₆ alkyl substituted with one or more —OSi(R¹³)₃, and R^{1b} is —SO₂NR¹¹R¹²; R^{1a} is C₁-C₆ alkyl substituted with one or more —OSi(R¹³)₃, and R^{1b} is —SO₂R¹³; R^{1a} is C₁-C₆ alkyl substituted with one or more —OSi(R¹³)₃, and R^{1b} is —CONR¹¹R¹²; R^{1a} is C₁-C₆ alkyl substituted with one or more —OSi(R¹³)₃, and R^{1b} is —OR¹¹; R^{1a} is C₁-C₆ alkyl substituted with one or more —OSi(R¹³)₃, and R^{1b} is —COR¹³; R^{1a} is C₁-C₆ alkyl substituted with one or more —OSi(R¹³)₃, and R^{1b} is —CO₂R¹³; R^{1a} is C₁-C₆ alkyl substituted with one or more —OSi(R¹³)₃, and R^{1b} is —NR¹³CONR¹¹R¹²; R^{1a} is C₁-C₆ alkyl substituted with one or more —OSi(R¹³)₃, and R^{1b} is —CR¹¹R¹²CN; R^{1a} is C₁-C₆ alkyl substituted with one or more —OSi(R¹³)₃, and R^{1b} is —NR¹¹SO₂R¹³; R^{1a} is C₁-C₆ alkyl substituted with one or more —OSi(R¹³)₃, and R^{1b} is —NR¹¹CONR¹¹R¹²; R^{1a} is C₁-C₆ alkyl substituted with one or more —OSi(R¹³)₃, and R^{1b} is —CN; R^{1a} is C₁-C₆ alkyl substituted with one or more —OSi(R¹³)₃, and R^{1b} is —NR¹¹COR¹²; R^{1a} is C₁-C₆ alkyl substituted with one or more —OSi(R¹³)₃, and R^{1b} is —CR¹¹R¹²NR¹¹R¹²; R^{1a} is —SO₂NR¹¹R¹², and R^{1b} is C₁-C₆ alkyl substituted with one or more hydroxy; R^{1a} is —SO₂NR¹¹R¹², and R^{1b} is —SO₂NR¹¹R¹²; R^{1a} is —SO₂NR¹¹R¹², and R^{1b} is —SO₂R¹³; R^{1a} is —SO₂NR¹¹R¹², and R^{1b} is —CONR¹¹R¹²; R^{1a} is —SO₂NR¹¹R¹², and R^{1b} is —OR¹¹; R^{1a} is —SO₂NR¹¹R¹², and R^{1b} is —COR¹³; R^{1a} is —SO₂NR¹¹R¹², and R^{1b} is —CO₂R¹³; R^{1a} is —SO₂NR¹¹R¹², and R^{1b} is —NR¹³CONR¹¹R¹²; R^{1a} is —SO₂NR¹¹R¹², and R^{1b} is —CR¹¹R¹²CN; R^{1a} is —SO₂NR¹¹R¹², and R^{1b} is —NR¹¹SO₂R¹³; R^{1a} is —SO₂NR¹¹R¹², and R^{1b} is —CR¹¹R¹²NR¹¹R¹²; R^{1a} is —SO₂NR¹¹R¹², and R^{1b} is —CN; R^{1a} is —SO₂NR¹¹R¹², and R^{1b} is —NR¹¹CONR¹¹R¹²; R^{1a} is —SO₂NR¹¹R¹², and R^{1b} is —NR¹¹COR¹²; R^{1a} is —CR¹¹R¹²NR¹¹R¹², and R^{1b} is C₁-C₆ alkyl substituted with one or more hydroxy; R^{1a} is —CR¹¹R¹²NR¹¹R¹², and R^{1b} is —SO₂NR¹¹R¹²; R^{1a} is —CR¹¹R¹²NR¹¹R¹², and R^{1b} is —SO₂R¹³; R^{1a} is —CR¹¹R¹²NR¹¹R¹², and R^{1b} is —CONR¹¹R¹²; R^{1a} is —CR¹¹R¹²NR¹¹R¹², and R^{1b} is —OR¹¹; R^{1a} is —CR¹¹R¹²NR¹¹R¹², and R^{1b} is —COR¹³; R^{1a} is —CR¹¹R¹²NR¹¹R¹², and R^{1b} is —CO₂R¹³; R^{1a} is —CR¹¹R¹²NR¹¹R¹², and R^{1b} is —NR¹³CONR¹¹R¹²; R^{1a} is —CR¹¹R¹²NR¹¹R¹², and R^{1b} is —CR¹¹R¹²CN; R^{1a} is —CR¹¹R¹²NR¹¹R¹², and R^{1b} is —NR¹¹SO₂R¹³; R^{1a} is —CR¹¹R¹²NR¹¹R¹², and R^{1b} is —CR¹¹R¹²NR¹¹R¹²; R^{1a} is —CR¹¹R¹²NR¹¹R¹², and R^{1b} is —CN; R^{1a} is —CR¹¹R¹²NR¹¹R¹², and R^{1b} is —NR¹¹CONR¹¹R¹²; and R^{1a} is —CR¹¹R¹²NR¹¹R¹², and R^{1b} is —NR¹¹COR¹².

In some embodiments of the compound of formula AA,

the substituted ring A is

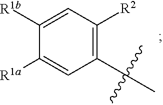

R^{1a} and R^{1b} are one of the following combinations:

In some embodiments, R^{1a} is 2-hydroxy-2-propyl, and R^{1b} is —OMe; In some embodiments, R^{1a} is 2-hydroxy-2-propyl, and R^{1b} is —OH; In some embodiments, R^{1a} is 2-hydroxy-2-propyl, and R^{1b} is —CO₂Me; In some embodiments, R^{1a} is 2-hydroxy-2-propyl, and R^{1b} is hydroxymethyl; In some embodiments, R^{1a} is 2-hydroxy-2-propyl, and R^{1b} is hydroxy ethyl; In some embodiments, R^{1a} is 2-hydroxy-2-propyl, and R^{1b} is 2-hydroxy-2-propyl; In some embodiments, R^{1a} is 2-hydroxy-2-propyl, and R^{1b} is —SO₂NHCH₂CH₂OH; In some embodiments, R^{1a} is 2-hydroxy-2-propyl, and R^{1b} is —SO₂Me; In some embodiments, R^{1a} is 2-hydroxy-2-propyl, and R^{1b} is CONHMe; In some embodiments, R^{1a} is 2-hydroxy-2-propyl, and R^{1b} is cyanomethyl; In some embodiments, R^{1a} is 2-hydroxy-2-propyl, and R^{1b} is —CN; In some embodiments, R^{1a} is 2-hydroxy-2-propyl, and R^{1b} is dimethylaminomethyl; In some embodiments, R^{1a} is dimethylaminomethyl, and R^{1b} is —OMe; In some embodiments, R^{1a} is dimethylaminomethyl, and R^{1b} is —OH; In some embodiments, R^{1a} is dimethylaminomethyl, and R^{1b} is —CO₂Me; In some embodiments, R^{1a} is dimethylaminomethyl, and R^{1b} is hydroxymethyl; In some embodiments, R^{1a} is dimethylaminomethyl, and R^{1b} is hydroxy ethyl; In some embodiments, R^{1a} is dimethylaminomethyl, and R^{1b} is 2-hydroxy-2-propyl; In some embodiments, R^{1a} is dimethylaminomethyl, and R^{1b} is —SO₂NHCH₂CH₂OH; In some embodiments, R^{1a} is dimethylaminomethyl, and R^{1b} is —SO₂Me; In some embodiments, R^{1a} is dimethylaminomethyl, and R^{1b} is CONHMe; In some embodiments, R^{1a} is dimethylaminomethyl, and R^{1b} is cyanomethyl; In some embodiments, R^{1a} is dimethylaminomethyl, and R^{1b} is dimethylaminomethyl; In some embodiments, R^{1a} is dimethylaminomethyl, and R^{1b} is CN; In some embodiments, R^{1a} is —SO₂NHMe, and R^{1b} is —OMe; In some embodiments, R^{1a} is —SO₂NHMe, and R^{1b} is —OH; In some embodiments, R^{1a} is —SO₂NHMe, and R^{1b} is —CO₂Me; In some embodiments, R^{1a} is —SO₂NHMe, and R^{1b} is hydroxymethyl; In some embodiments, R^{1a} is —SO₂NHMe, and R^{1b} is hydroxyethyl; In some embodiments, R^{1a} is —SO₂NHMe, and R^{1b} is 2-hydroxy-2-propyl; In some embodiments, R^{1a} is —SO₂NHMe, and R^{1b} is —SO₂NHCH₂CH₂OH; In some embodiments, R^{1a} is —SO₂NHMe, and R^{1b} is —SO₂Me; In some embodiments, R^{1a} is —SO₂NHMe, and R^{1b} is CONHMe; In some embodiments, R^{1a} is —SO₂NHMe, and R^{1b} is dimethylaminomethyl; In some embodiments, R^{1a} is —SO₂NHMe, and R^{1b} is —CN; In some embodiments, R^{1a} is —SO₂NHMe, and R^{1b} is cyanomethyl; and In some embodiments, R^{1a} is C₁-C₄ alkyl substituted with one —OSi(Me)₂tBu, and R^{1b} is —CO₂Me.

In some embodiments of the compound of formula AA,

the substituted ring A is

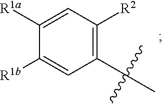

R^{1a} and R^{1b} are one of the following combinations:
R^{1a} is C₁-C₆ alkyl substituted with one or more hydroxy, and R^{1b} is C₁-C₆ alkyl substituted with one or more hydroxy; R^{1a} is C₁-C₆ alkyl substituted with one or more hydroxy, and R^{1b} is —SO₂NR¹¹R¹²; R^{1a} is C₁-C₆ alkyl substituted with one or more hydroxy, and R^{1b} is —SO₂R¹³; R^{1a} is C₁-C₆ alkyl substituted with one or more hydroxy, and R^{1b} is —CONR¹¹R¹²; R^{1a} is C₁-C₆ alkyl substituted with one or more hydroxy, and R^{1b} is —OR¹¹; R^{1a} is C₁-C₆ alkyl substituted with one or more hydroxy, and R^{1b} is —COR¹³; R^{1a} is C₁-C₆ alkyl substituted with one or more hydroxy, and R^{1b} is —CO₂R¹³; R^{1a} is C₁-C₆ alkyl substituted with one or more hydroxy, and R^{1b} is —NR¹³CONR¹¹R¹²; R^{1a} is C₁-C₆ alkyl substituted with one or more hydroxy, and R^{1b} is —CR¹¹R¹²CN; R^{1a} is C₁-C₆ alkyl substituted with one or more hydroxy, and R^{1b} is —NR¹¹SO₂R¹³; R^{1a} is C₁-C₆ alkyl substituted with one or more hydroxy, and R^{1b} is —NR¹¹CONR¹¹R¹²; R^{1a} is C₁-C₆ alkyl substituted with one or more hydroxy, and R^{1b} is —CN; R^{1a} is C₁-C₆ alkyl substituted with one or more hydroxy, and R^{1b} is —NR¹¹COR¹²; R^{1a} is C₁-C₆ alkyl substituted with one or more hydroxy, and R^{1b} is —CR¹¹R¹²NR¹¹R¹²; R^{1a} is C₁-C₆ alkyl substituted with one or more —OSi(R¹³)₃, and R^{1b} is C₁-C₆ alkyl substituted with one or more hydroxy; R^{1a} is C₁-C₆ alkyl substituted with one or more —OSi(R¹³)₃, and R^{1b} is —SO₂NR¹¹R¹²; R^{1a} is C₁-C₆ alkyl substituted with one or more —OSi(R¹³)₃, and R^{1b} is —SO₂R¹³; R^{1a} is C₁-C₆ alkyl substituted with one or more —OSi(R¹³)₃, and R^{1b} is —CONR¹¹R¹²; R^{1a} is C₁-C₆ alkyl substituted with one or more —OSi(R¹³)₃, and R^{1b} is —OR¹¹; R^{1a} is C₁-C₆ alkyl substituted with one or more —OSi(R¹³)₃, and R^{1b} is —COR¹³; R^{1a} is C₁-C₆ alkyl substituted with one or more —OSi(R¹³)₃, and R^{1b} is —CO₂R¹³; R^{1a} is C₁-C₆ alkyl substituted with one or more —OSi(R¹³)₃, and R^{1b} is —NR¹³CONR¹¹R¹²; R^{1a} is C₁-C₆ alkyl substituted with one or more —OSi(R¹³)₃, and R^{1b} is —CR¹¹R¹²CN; R^{1a} is C₁-C₆ alkyl substituted with one or more —OSi(R¹³)₃, and R^{1b} is —NR¹¹SO₂R¹³; R^{1a} is C₁-C₆ alkyl substituted with one or more —OSi(R¹³)₃, and R^{1b} is —NR¹¹CONR¹¹R¹²; R^{1a} is C₁-C₆ alkyl substituted with one or more —OSi(R¹³)₃, and R^{1b} is —CN; R^{1a} is C₁-C₆ alkyl substituted with one or more —OSi(R¹³)₃, and R^{1b} is —NR¹¹COR¹²; R^{1a} is C₁-C₆ alkyl substituted with one or more —OSi(R¹³)₃, and R^{1b} is —CR¹¹R¹²NR¹¹R¹²; R^{1a} is —SO₂NR¹¹R¹², and R^{1b} is C₁-C₆ alkyl substituted with one or more hydroxy; R^{1a} is —SO₂NR¹¹R¹², and R^{1b} is —SO₂NR¹¹R¹²; R^{1a} is —SO₂NR¹¹R¹², and R^{1b} is —SO₂R¹³; R^{1a} is —SO₂NR¹¹R¹², and R^{1b} is —CONR¹¹R¹²; R^{1a} is —SO₂NR¹¹R¹², and R^{1b} is —OR¹¹; R^{1a} is —SO₂NR¹¹R¹², and R^{1b} is —COR¹³; R^{1a} is —SO₂NR¹¹R¹², and R^{1b} is —CO₂R¹³; R^{1a} is —SO₂NR¹¹R¹², and R^{1b} is —NR¹³CONR¹¹R¹²; R^{1a} is —SO₂NR¹¹R¹², and R^{1b} is —CR¹¹R¹²CN; R^{1a} is —SO₂NR¹¹R¹², and R^{1b} is —NR¹¹SO₂R¹³; R^{1a} is —SO₂NR¹¹R¹², and R^{1b} is —CR¹¹R¹²NR¹¹R¹²; R^{1a} is —SO₂NR¹¹R¹², and R^{1b} is —CN; R^{1a} is —SO₂NR¹¹R¹², and R^{1b} is —NR¹¹CONR¹¹R¹²; R^{1a} is —SO₂NR¹¹R¹², and R^{1b} is —NR¹¹COR¹²; R^{1a} is —CR¹¹R¹²NR¹¹R¹², and R^{1b} is C₁-C₆ alkyl substituted with one or more hydroxy; R^{1a} is —CR¹¹R¹²NR¹¹R¹², and R^{1b} is —SO₂NR¹¹R¹²; R^{1a} is —CR¹¹R¹²NR¹¹R¹², and R^{1b} is —SO₂R¹³; R^{1a} is —CR¹¹R¹²NR¹¹R¹², and R^{1b} is —CONR¹¹R¹²; R^{1a} is —CR¹¹R¹²NR¹¹R¹², and R^{1b} is —OR¹¹; R^{1a} is —CR¹¹R¹²NR¹¹R¹², and R^{1b} is —COR¹³; R^{1a} is —CR¹¹R¹²NR¹¹R¹², and R^{1b} is —CO₂R¹³; R^{1a} is —CR¹¹R¹²NR¹¹R¹², and R^{1b} is —NR¹³CONR¹¹R¹²; R^{1a} is —CR¹¹R¹²NR¹¹R¹², and R^{1b} is —CR¹¹R¹²CN; R^{1a} is —CR¹¹R¹²NR¹¹R¹², and R^{1b} is —NR¹¹SO₂R¹³; R^{1a} is —CR¹¹R¹²NR¹¹R¹², and R^{1b} is —CR¹¹R¹²NR¹¹R¹²; R^{1a} is —CR¹¹R¹²NR¹¹R¹², and R^{1b} is —CN; R^{1a} is —CR¹¹R¹²NR¹¹R¹², and R^{1b} is —NR¹¹CONR¹¹R¹²; and R^{1a} is —CR¹¹R¹²NR¹¹R¹², and R^{1b} is —NR¹¹COR¹².

In some embodiments of the compound of formula AA,

the substituted ring A is

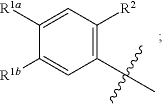

R^{1a} and R^{1b} are one of the following combinations:

In some embodiments, R^{1a} is 2-hydroxy-2-propyl, and R^{1b} is —OMe; In some embodiments, R^{1a} is 2-hydroxy-2-propyl, and R^{1b} is —OH; In some embodiments, R^{1a} is 2-hydroxy-2-propyl, and R^{1b} is —CO₂Me; In some embodiments, R^{1a} is 2-hydroxy-2-propyl, and R^{1b} is hydroxymethyl; In some embodiments, R^{1a} is 2-hydroxy-2-propyl, and R^{1b} is hydroxy ethyl; In some embodiments, R^{1a} is 2-hydroxy-2-propyl, and R^{1b} is 2-hydroxy-2-propyl; In some embodiments, R^{1a} is 2-hydroxy-2-propyl, and R^{1b} is —SO₂NHCH₂CH₂OH; In some embodiments, R^{1a} is 2-hydroxy-2-propyl, and R^{1b} is —SO₂Me; In some embodiments, R^{1a} is 2-hydroxy-2-propyl, and R^{1b} is CONHMe; In some embodiments, R^{1a} is 2-hydroxy-2-propyl, and R^{1b} is cyanomethyl; In some embodiments, R^{1a} is 2-hydroxy-2-propyl, and R^{1b} is —CN; In some embodiments, R^{1a} is 2-hydroxy-2-propyl, and R^{1b} is dimethylaminomethyl; In some embodiments, R^{1a} is dimethylaminomethyl, and R^{1b} is —OMe; In some embodiments, R^{1a} is dimethylaminomethyl, and R^{1b} is —OH; In some embodiments, R^{1a} is dimethylaminomethyl, and R^{1b} is —CO₂Me; In some embodiments, R^{1a} is dimethylaminomethyl, and R^{1b} is hydroxymethyl; In some embodiments, R^{1a} is dimethylaminomethyl, and R^{1b} is hydroxy ethyl; In some embodiments, R^{1a} is dimethylaminomethyl, and R^{1b} is 2-hydroxy-2-propyl; In some embodiments, R^{1a} is dimethylaminomethyl, and R^{1b} is —SO₂NHCH₂CH₂OH; In some embodiments, R^{1a} is dimethylaminomethyl, and R^{1b} is —SO₂Me; In some embodiments, R^{1a} is dimethylaminomethyl, and R^{1b} is CONHMe; In some embodiments, R^{1a} is dimethylaminomethyl, and R^{1b} is cyanomethyl; In some embodiments, R^{1a} is dimethylaminomethyl, and R^{1b} is dimethylaminomethyl; In some embodiments, R^{1a} is dimethylaminomethyl, and R^{1b} is CN; In some embodiments, R^{1a} is —SO₂NHMe, and R^{1b} is —OMe; In some embodiments, R^{1a} is —SO₂NHMe, and R^{1b} is —OH; In some embodiments, R^{1a} is —SO₂NHMe, and R^{1b} is —CO₂Me; In some embodiments, R^{1a} is —SO₂NHMe, and R^{1b} is hydroxymethyl; In some embodiments, R^{1a} is —SO₂NHMe, and R^{1b} is hydroxyethyl; In some embodiments, R^{1a} is —SO₂NHMe, and R^{1b} is 2-hydroxy-2-propyl; In some embodiments, R^{1a} is —SO₂NHMe, and R^{1b} is —SO₂NHCH₂CH₂OH; In some embodiments, R^{1a} is —SO₂NHMe, and R^{1b} is —SO₂Me; In some embodiments, R^{1a} is —SO₂NHMe, and R^{1b} is CONHMe; In some embodiments, R^{1a} is —SO₂NHMe, and R^{1b} is dimethylaminomethyl; In some embodiments, R^{1a} is —SO₂NHMe, and R^{1b} is —CN; In some embodiments, R^{1a} is —SO₂NHMe, and R^{1b} is cyanomethyl; and In some embodiments, R^{1a} is C₁-C₄ alkyl substituted with one —OSi(Me)₂tBu, and R^{1b} is —CO₂Me.

In some embodiments of the compound of formula AA,

the substituted ring A is

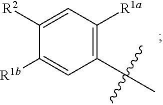

R^{1a} and R^{1b} are one of the following combinations:
R^{1a} is C₁-C₆ alkyl substituted with one or more hydroxy, and R^{1b} is C₁-C₆ alkyl substituted with one or more hydroxy; R^{1a} is C₁-C₆ alkyl substituted with one or more hydroxy, and R^{1b} is —SO₂NR¹¹R¹²; R^{1a} is C₁-C₆ alkyl substituted with one or more hydroxy, and R^{1b} is —SO₂R¹³; R^{1a} is C₁-C₆ alkyl substituted with one or more hydroxy, and R^{1b} is —CONR¹¹R¹²; R^{1a} is C₁-C₆ alkyl substituted with one or more hydroxy, and R^{1b} is —OR¹¹; R^{1a} is C₁-C₆ alkyl substituted with one or more hydroxy, and R^{1b} is —COR¹³; R^{1a} is C₁-C₆ alkyl substituted with one or more hydroxy, and R^{1b} is —CO₂R¹³; R^{1a} is C₁-C₆ alkyl substituted with one or more hydroxy, and R^{1b} is —NR¹³CONR¹¹R¹²; R^{1a} is C₁-C₆ alkyl substituted with one or more hydroxy, and R^{1b} is —CR¹¹R¹²CN; R^{1a} is C₁-C₆ alkyl substituted with one or more hydroxy, and R^{1b} is —NR¹¹SO₂R¹³; R^{1a} is C₁-C₆ alkyl substituted with one or more hydroxy, and R^{1b} is —NR¹¹CONR¹¹R¹²; R^{1a} is C₁-C₆ alkyl substituted with one or more hydroxy, and R^{1b} is —CN; R^{1a} is C₁-C₆ alkyl substituted with one or more hydroxy, and R^{1b} is —NR¹¹COR¹²; R^{1a} is C₁-C₆ alkyl substituted with one or more hydroxy, and R^{1b} is —CR¹¹R¹²NR¹¹R¹²; R^{1a} is C₁-C₆ alkyl substituted with one or more —OSi(R¹³)₃, and R^{1b} is C₁-C₆ alkyl substituted with one or more hydroxy; R^{1a} is C₁-C₆ alkyl substituted with one or more —OSi(R¹³)₃, and R^{1b} is —SO₂NR¹¹R¹²; R^{1a} is C₁-C₆ alkyl substituted with one or more —OSi(R¹³)₃, and R^{1b} is —SO₂R¹³; R^{1a} is C₁-C₆ alkyl substituted with one or more —OSi(R¹³)₃, and R^{1b} is —CONR¹¹R¹²; R^{1a} is C₁-C₆ alkyl substituted with one or more —OSi(R¹³)₃, and R^{1b} is —OR¹¹; R^{1a} is C₁-C₆ alkyl substituted with one or more —OSi(R¹³)₃, and R^{1b} is —COR¹³; R^{1a} is C₁-C₆ alkyl substituted with one or more —OSi(R¹³)₃, and R^{1b} is —CO₂R¹³; R^{1a} is C₁-C₆ alkyl substituted with one or more —OSi(R¹³)₃, and R^{1b} is —NR¹³CONR¹¹R¹²; R^{1a} is C₁-C₆ alkyl substituted with one or more —OSi(R¹³)₃, and R^{1b} is —CR¹¹R¹²CN; R^{1a} is C₁-C₆ alkyl substituted with one or more —OSi(R¹³)₃, and R^{1b} is —NR¹¹SO₂R¹³; R^{1a} is C₁-C₆ alkyl substituted with one or more —OSi(R¹³)₃, and R^{1b} is —NR¹¹CONR¹¹R¹²; R^{1a} is C₁-C₆ alkyl substituted with one or more —OSi(R¹³)₃, and R^{1b} is —CN; R^{1a} is C₁-C₆ alkyl substituted with one or more —OSi(R¹³)₃, and R^{1b} is —NR¹¹COR¹²; R^{1a} is C₁-C₆ alkyl substituted with one or more —OSi(R¹³)₃, and R^{1b} is —CR¹¹R¹²NR¹¹R¹²; R^{1a} is —SO₂NR¹¹R¹², and R^{1b} is C₁-C₆ alkyl substituted with one or more hydroxyl; R^{1a} is —SO₂NR¹¹R¹², and R^{1b} is —SO₂NR¹¹R¹²; R^{1a} is —SO₂NR¹¹R¹², and R^{1b} is —SO₂R¹³; R^{1a} is —SO₂NR¹¹R¹², and R^{1b} is —CONR¹¹R¹²; R^{1a} is —SO₂NR¹¹R¹², and R^{1b} is —OR¹¹; R^{1a} is —SO₂NR¹¹R¹², and R^{1b} is —COR¹³; R^{1a} is —SO₂NR¹¹R¹², and R^{1b} is —CO₂R¹³; R^{1a} is —SO₂NR¹¹R¹², and R^{1b} is —NR¹³CONR¹¹R¹²; R^{1a} is —SO₂NR¹¹R¹², and R^{1b} is —CR¹¹R¹²CN; R^{1a} is —SO₂NR¹¹R¹², and R^{1b} is —NR¹¹SO₂R¹³; R^{1a} is —SO₂NR¹¹R¹², and R^{1b} is —CR¹¹R¹²NR¹¹R¹²; R^{1a} is —SO₂NR¹¹R¹², and R^{1b} is —CN; R^{1a} is —SO₂NR¹¹R¹², and R^{1b} is —NR¹¹CONR¹¹R¹²; R^{1a} is —SO₂NR¹¹R¹², and R^{1b} is —NR¹¹COR¹²; R^{1a} is —CR¹¹R¹²NR¹¹R¹², and R^{1b} is C₁-C₆ alkyl substituted with one or more hydroxy; R^{1a} is —CR¹¹R¹²NR¹¹R¹², and R^{1b} is —SO₂NR¹¹R¹²; R^{1a} is —CR¹¹R¹²NR¹¹R¹², and R^{1b} is —SO₂R¹³; R^{1a} is —CR¹¹R¹²NR¹¹R¹², and R^{1b} is —CONR¹¹R¹²; R^{1a} is —CR¹¹R¹²NR¹¹R¹², and R^{1b} is —OR¹¹; R^{1a} is —CR¹¹R¹²NR¹¹R¹², and R^{1b} is —COR¹³; R^{1a} is —CR¹¹R¹²NR¹¹R¹², and R^{1b} is —CO₂R¹³; R^{1a} is —CR¹¹R¹²NR¹¹R¹², and R^{1b} is —NR¹³CONR¹¹R¹²; R^{1a} is —CR¹¹R¹²NR¹¹R¹², and R^{1b} is —CR¹¹R¹²CN; R^{1a} is —CR¹¹R¹²NR¹¹R¹², and R^{1b} is —NR¹¹SO₂R¹³; R^{1a} is —CR¹¹R¹²NR¹¹R¹², and R^{1b} is —CR¹¹R¹²NR¹¹R¹²; R^{1a} is —CR¹¹R¹²NR¹¹R¹², and R^{1b} is —CN; R^{1a} is —CR¹¹R¹²NR¹¹R¹², and R^{1b} is —NR¹¹CONR¹¹R¹²; and R^{1a} is —CR¹¹R¹²NR¹¹R¹², and R^{1b} is —NR¹¹COR¹².

In some embodiments of the compound of formula AA,

the substituted ring A is

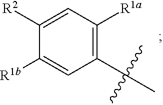

R^{1a} and R^{1b} are one of the following combinations:

In some embodiments, R^{1a} is 2-hydroxy-2-propyl, and R^{1b} is —OMe; In some embodiments, R^{1a} is 2-hydroxy-2-propyl, and R^{1b} is —OH; In some embodiments, R^{1a} is 2-hydroxy-2-propyl, and R^{1b} is —CO₂Me; In some embodiments, R^{1a} is 2-hydroxy-2-propyl, and R^{1b} is hydroxymethyl; In some embodiments, R^{1a} is 2-hydroxy-2-propyl, and R^{1b} is hydroxy ethyl; In some embodiments, R^{1a} is 2-hydroxy-2-propyl, and R^{1b} is 2-hydroxy-2-propyl; In some embodiments, R^{1a} is 2-hydroxy-2-propyl, and R^{1b} is —SO₂NHCH₂CH₂OH; In some embodiments, R^{1a} is 2-hydroxy-2-propyl, and R^{1b} is —SO₂Me; In some embodiments, R^{1a} is 2-hydroxy-2-propyl, and R^{1b} is CONHMe; In some embodiments, R^{1a} is 2-hydroxy-2-propyl, and R^{1b} is cyanomethyl; In some embodiments, R^{1a} is 2-hydroxy-2-propyl, and R^{1b} is —CN; In some embodiments, R^{1a} is 2-hydroxy-2-propyl, and R^{1b} is dimethylaminomethyl; In some embodiments, R^{1a} is dimethylaminomethyl, and R^{1b} is —OMe; In some embodiments, R^{1a} is dimethylaminomethyl, and R^{1b} is —OH; In some embodiments, R^{1a} is dimethylaminomethyl, and R^{1b} is —CO₂Me; In some embodiments, R^{1a} is dimethylaminomethyl, and R^{1b} is hydroxymethyl; In some embodiments, R^{1a} is dimethylaminomethyl, and R^{1b} is hydroxy ethyl; In some embodiments, R^{1a} is dimethylaminomethyl, and R^{1b} is 2-hydroxy-2-propyl; In some embodiments, R^{1a} is dimethylaminomethyl, and R^{1b} is —SO₂NHCH₂CH₂OH; In some embodiments, R^{1a} is dimethylaminomethyl, and R^{1b} is —SO₂Me; In some embodiments, R^{1a} is dimethylaminomethyl, and R^{1b} is CONHMe; In some embodiments, R^{1a} is dimethylaminomethyl, and R^{1b} is cyanomethyl; In some embodiments, R^{1a} is dimethylaminomethyl, and R^{1b} is dimethylaminomethyl; In some embodiments, R^{1a} is dimethylaminomethyl, and R^{1b} is CN; In some embodiments, R^{1a} is —SO₂NHMe, and R^{1b} is —OMe; In some embodiments, R^{1a} is —SO₂NHMe, and R^{1b} is —OH; In some embodiments, R^{1a} is —SO₂NHMe, and R^{1b} is —CO₂Me; In some embodiments, R^{1a} is —SO₂NHMe, and R^{1b} is hydroxymethyl; In some embodiments, R^{1a} is —SO₂NHMe, and R^{1b} is hydroxyethyl; In some embodiments, R^{1a} is —SO₂NHMe, and R^{1b} is 2-hydroxy-2-propyl; In some embodiments, R^{1a} is —SO₂NHMe, and R^{1b} is —SO₂NHCH₂CH₂OH; In some embodiments, R^{1a} is —SO₂NHMe, and R^{1b} is —SO₂Me; In some embodiments, R^{1a} is —SO₂NHMe, and R^{1b} is CONHMe; In some embodiments, R^{1a} is —SO₂NHMe, and R^{1b} is dimethylaminomethyl; In some embodiments, R^{1a} is —SO₂NHMe, and R^{1b} is —CN; In some embodiments, R^{1a} is —SO₂NHMe, and R^{1b} is cyanomethyl; and In some embodiments, R^{1a} is C₁-C₄ alkyl substituted with one —OSi(Me)₂tBu, and R^{1b} is —CO₂Me.

In some embodiments of the compound of formula AA,

the substituted ring A is

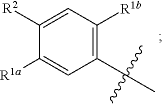

R^{1a} and R^{1b} are one of the following combinations:
R^{1a} is C₁-C₆ alkyl substituted with one or more hydroxy, and R^{1b} is C₁-C₆ alkyl substituted with one or more hydroxy; R^{1a} is C₁-C₆ alkyl substituted with one or more hydroxy, and R^{1b} is —SO₂NR¹¹R¹²; R^{1a} is C₁-C₆ alkyl substituted with one or more hydroxy, and R^{1b} is —SO₂R¹³; R^{1a} is C₁-C₆ alkyl substituted with one or more hydroxy, and R^{1b} is —CONR¹¹R¹²; R^{1a} is C₁-C₆ alkyl substituted with one or more hydroxy, and R^{1b} is —OR¹¹; R^{1a} is C₁-C₆ alkyl substituted with one or more hydroxy, and R^{1b} is —COR¹³; R^{1a} is C₁-C₆ alkyl substituted with one or more hydroxy, and R^{1b} is —CO₂R¹³; R^{1a} is C₁-C₆ alkyl substituted with one or more hydroxy, and R^{1b} is —NR¹³CONR¹¹R¹²; R^{1a} is C₁-C₆ alkyl substituted with one or more hydroxy, and R^{1b} is —CR¹¹R¹²CN; R^{1a} is C₁-C₆ alkyl substituted with one or more hydroxy, and R^{1b} is —NR¹¹SO₂R¹³; R^{1a} is C₁-C₆ alkyl substituted with one or more hydroxy, and R^{1b} is —NR¹¹CONR¹¹R¹²; R^{1a} is C₁-C₆ alkyl substituted with one or more hydroxy, and R^{1b} is —CN; R^{1a} is C₁-C₆ alkyl substituted with one or more hydroxy, and R^{1b} is —NR¹¹COR¹²; R^{1a} is C₁-C₆ alkyl substituted with one or more hydroxy, and R^{1b} is —CR¹¹R¹²NR¹¹R¹²; R^{1a} is C₁-C₆ alkyl substituted with one or more —OSi(R¹³)₃, and R^{1b} is C₁-C₆ alkyl substituted with one or more hydroxy; R^{1a} is C₁-C₆ alkyl substituted with one or more —OSi(R¹³)₃, and R^{1b} is —SO₂NR¹¹R¹²; R^{1a} is C₁-C₆ alkyl substituted with one or more —OSi(R¹³)₃, and R^{1b} is —SO₂R¹³; R^{1a} is C₁-C₆ alkyl substituted with one or more —OSi(R¹³)₃, and R^{1b} is —CONR¹¹R¹²; R^{1a} is C₁-C₆ alkyl substituted with one or more —OSi(R¹³)₃, and R^{1b} is —OR¹¹; R^{1a} is C₁-C₆ alkyl substituted with one or more —OSi(R¹³)₃, and R^{1b} is —COR¹³; R^{1a} is C₁-C₆ alkyl substituted with one or more —OSi(R¹³)₃, and R^{1b} is —CO₂R¹³; R^{1a} is C₁-C₆ alkyl substituted with one or more —OSi(R¹³)₃, and R^{1b} is —NR¹³CONR¹¹R¹²; R^{1a} is C₁-C₆ alkyl substituted with one or more —OSi(R¹³)₃, and R^{1b} is —CR¹¹R¹²CN; R^{1a} is C₁-C₆ alkyl substituted with one or more —OSi(R¹³)₃, and R^{1b} is —NR¹¹SO₂R¹³; R^{1a} is C₁-C₆ alkyl substituted with one or more —OSi(R¹³)₃, and R^{1b} is —NR¹¹CONR¹¹R¹²; R^{1a} is C₁-C₆ alkyl substituted with one or more —OSi(R¹³)₃, and R^{1b} is —CN; R^{1a} is C₁-C₆ alkyl substituted with one or more —OSi(R¹³)₃, and R^{1b} is —NR¹¹COR¹²; R^{1a} is C₁-C₆ alkyl substituted with one or more —OSi(R¹³)₃, and R^{1b} is —CR¹¹R¹²NR¹¹R¹²; R^{1a} is —SO₂NR¹¹R¹², and R^{1b} is —SO₂NR¹¹R¹²; R^{1a} is —SO₂NR¹¹R¹², and R^{1b} is —SO₂R¹³; R^{1a} is —SO₂NR¹¹R¹², and R^{1b} is —CONR¹¹R¹²; R^{1a} is —SO₂NR¹¹R¹², and R^{1b} is —OR¹¹; R^{1a} is —SO₂NR¹¹R¹², and R^{1b} is —COR¹³; R^{1a} is —SO₂NR¹¹R¹², and R^{1b} is —CO₂R¹³; R^{1a} is —SO₂NR¹¹R¹², and R^{1b} is —NR¹³CONR¹¹R¹²; R^{1a} is —SO₂NR¹¹R¹², and R^{1b} is —CR¹¹R¹²CN; R^{1a} is —SO₂NR¹¹R¹², and R^{1b} is —NR¹¹SO₂R¹³; R^{1a} is —SO₂NR¹¹R¹², and R^{1b} is —CR¹¹R¹²NR¹¹R¹²; R^{1a} is —SO₂NR¹¹R¹², and R^{1b} is —CN; R^{1a} is —SO₂NR¹¹R¹², and R^{1b} is —NR¹¹CONR¹¹R¹²; R^{1a} is —SO₂NR¹¹R¹², and R^{1b} is —NR¹¹COR¹²; R^{1a} is —CR¹¹R¹²NR¹¹R¹², and R^{1b} is C₁-C₆ alkyl substituted with one or more hydroxy; R^{1a} is —CR¹¹R¹²NR¹¹R¹², and R^{1b} is —SO₂NR¹¹R¹²; R^{1a} is —CR¹¹R¹²NR¹¹R¹², and R^{1b} is —SO₂R¹³; R^{1a} is —CR¹¹R¹²NR¹¹R¹², and R^{1b} is —CONR¹¹R¹²; R^{1a} is —CR¹¹R¹²NR¹¹R¹², and R^{1b} is —OR¹¹; R^{1a} is —CR¹¹R¹²NR¹¹R¹², and R^{1b} is —COR¹³; R^{1a} is —CR¹¹R¹²NR¹¹R¹², and R^{1b} is —CO₂R¹³; R^{1a} is —CR¹¹R¹²NR¹¹R¹², and R^{1b} is —NR¹³CONR¹¹R¹²; R^{1a} is —CR¹¹R¹²NR¹¹R¹², and R^{1b} is —CR¹¹R¹²CN; R^{1a} is —CR¹¹R¹²NR¹¹R¹², and R^{1b} is —NR¹¹SO₂R¹³; R^{1a} is —CR¹¹R¹²NR¹¹R¹², and R^{1b} is —CR¹¹R¹²NR¹¹R¹²; R^{1a} is —CR¹¹R¹²NR¹¹R¹², and R^{1b} is —CN; R^{1a} is —CR¹¹R¹²NR¹¹R¹², and R^{1b} is —NR¹¹CONR¹¹R¹²; and R^{1a} is —CR¹¹R¹²NR¹¹R¹², and R^{1b} is —NR¹¹COR¹².

In some embodiments of the compound of formula AA,

the substituted ring A is

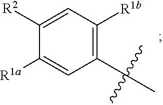

R^{1a} and R^{1b} are one of the following combinations:

In some embodiments, R^{1a} is 2-hydroxy-2-propyl, and R^{1b} is —OMe; In some embodiments, R^{1a} is 2-hydroxy-2-propyl, and R^{1b} is —OH; In some embodiments, R^{1a} is 2-hydroxy-2-propyl, and R^{1b} is —CO₂Me; In some embodiments, R^{1a} is 2-hydroxy-2-propyl, and R^{1b} is hydroxymethyl; In some embodiments, R^{1a} is 2-hydroxy-2-propyl, and R^{1b} is hydroxy ethyl; In some embodiments, R^{1a} is 2-hydroxy-2-propyl, and R^{1b} is 2-hydroxy-2-propyl; In some embodiments, R^{1a} is 2-hydroxy-2-propyl, and R^{1b} is —SO₂NHCH₂CH₂OH; In some embodiments, R^{1a} is 2-hydroxy-2-propyl, and R^{1b} is —SO₂Me; In some embodiments, R^{1a} is 2-hydroxy-2-propyl, and R^{1b} is CONHMe; In some embodiments, R^{1a} is 2-hydroxy-2-propyl, and R^{1b} is cyanomethyl; In some embodiments, R^{1a} is 2-hydroxy-2-propyl, and R^{1b} is —CN; In some embodiments, R^{1a} is 2-hydroxy-2-propyl, and R^{1b} is dimethylaminomethyl; In some embodiments, R^{1a} is dimethylaminomethyl, and R^{1b} is —OMe; In some embodiments, R^{1a} is dimethylaminomethyl, and R^{1b} is —OH; In some embodiments, R^{1a} is dimethylaminomethyl, and R^{1b} is —CO₂Me; In some embodiments, R^{1a} is dimethylaminomethyl, and R^{1b} is hydroxymethyl; In some embodiments, R^{1a} is dimethylaminomethyl, and R^{1b} is hydroxy ethyl; In some embodiments, R^{1a} is dimethylaminomethyl, and R^{1b} is 2-hydroxy-2-propyl; In some embodiments, R^{1a} is dimethylaminomethyl, and R^{1b} is —SO₂NHCH₂CH₂OH; In some embodiments, R^{1a} is dimethylaminomethyl, and R^{1b} is —SO₂Me; In some embodiments, R^{1a} is dimethylaminomethyl, and R^{1b} is CONHMe; In some embodiments, R^{1a} is dimethylaminomethyl, and R^{1b} is cyanomethyl; In some embodiments, R^{1a} is dimethylaminomethyl, and R^{1b} is dimethylaminomethyl; In some embodiments, R^{1a} is dimethylaminomethyl, and R^{1b} is CN In some embodiments, R^{1a} is —SO₂NHMe, and R^{1b} is —OMe; In some embodiments, R^{1a} is —SO₂NHMe, and R^{1b} is —OH; In some embodiments, R^{1a} is —SO₂NHMe, and R^{1b} is —CO₂Me; In some embodiments, R^{1a} is —SO₂NHMe, and R^{1b} is hydroxymethyl; In some embodiments, R^{1a} is —SO₂NHMe, and R^{1b} is hydroxyethyl; In some embodiments, R^{1a} is —SO₂NHMe, and R^{1b} is 2-hydroxy-2-propyl; In some embodiments, R^{1a} is —SO₂NHMe, and R^{1b} is —SO₂NHCH₂CH₂OH; In some embodiments, R^{1a} is —SO₂NHMe, and R^{1b} is —SO₂Me; In some embodiments, R^{1a} is —SO₂NHMe, and R^{1b} is CONHMe; In some embodiments, R^{1a} is —SO₂NHMe, and R^{1b} is dimethylaminomethyl; In some embodiments, R^{1a} is —SO₂NHMe, and R^{1b} is —CN; In some embodiments, R^{1a} is —SO₂NHMe, and R^{1b} is cyanomethyl; and In some embodiments, R^{1a} is C₁-C₄ alkyl substituted with one —OSi(Me)₂tBu, and R^{1b} is —CO₂Me.

In some embodiments of the compound of formula AA,

the substituted ring A is

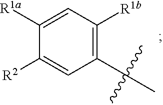

R^{1a} and R^{1b} are one of the following combinations:
R^{1a} is C₁-C₆ alkyl substituted with one or more hydroxy, and R^{1b} is C₁-C₆ alkyl substituted with one or more hydroxy; R^{1a} is C₁-C₆ alkyl substituted with one or more hydroxy, and R^{1b} is —SO₂NR¹¹R¹²; R^{1a} is C₁-C₆ alkyl substituted with one or more hydroxy, and R^{1b} is —SO₂R¹³; R^{1a} is C₁-C₆ alkyl substituted with one or more hydroxy, and R^{1b} is —CONR¹¹R¹²; R^{1a} is C₁-C₆ alkyl substituted with one or more hydroxy, and R^{1b} is —OR¹¹; R^{1a} is C₁-C₆ alkyl substituted with one or more hydroxy, and R^{1b} is —COR¹³; R^{1a} is C₁-C₆ alkyl substituted with one or more hydroxy, and R^{1b} is —CO₂R¹³; R^{1a} is C₁-C₆ alkyl substituted with one or more hydroxy, and R^{1b} is —NR¹³CONR¹¹R¹²; R^{1a} is C₁-C₆ alkyl substituted with one or more hydroxy, and R^{1b} is —CR¹¹R¹²CN; R^{1a} is C₁-C₆ alkyl substituted with one or more hydroxy, and R^{1b} is —NR¹¹SO₂R¹³; R^{1a} is C₁-C₆ alkyl substituted with one or more hydroxy, and R^{1b} is —NR¹¹CONR¹¹R¹²; R^{1a} is C₁-C₆ alkyl substituted with one or more hydroxy, and R^{1b} is —CN; R^{1a} is C₁-C₆ alkyl substituted with one or more hydroxy, and R^{1b} is —NR¹¹COR¹²; R^{1a} is C₁-C₆ alkyl substituted with one or more hydroxy, and R^{1b} is —CR¹¹R¹²NR¹¹R¹²; R^{1a} is C₁-C₆ alkyl substituted with one or more —OSi(R¹³)₃, and R^{1b} is C₁-C₆ alkyl substituted with one or more hydroxy; R^{1a} is C₁-C₆ alkyl substituted with one or more —OSi(R¹³)₃, and R^{1b} is —SO₂NR¹¹R¹²; R^{1a} is C₁-C₆ alkyl substituted with one or more —OSi(R¹³)₃, and R^{1b} is —SO₂R¹³; R^{1a} is C₁-C₆ alkyl substituted with one or more —OSi(R¹³)₃, and R^{1b} is —CONR¹¹R¹²; R^{1a} is C₁-C₆ alkyl substituted with one or more —OSi(R¹³)₃, and R^{1b} is —OR¹¹; R^{1a} is C₁-C₆ alkyl substituted with one or more —OSi(R¹³)₃, and R^{1b} is —COR¹³; R^{1a} is C₁-C₆ alkyl substituted with one or more —OSi(R¹³)₃, and R^{1b} is —CO₂R¹³; R^{1a} is C₁-C₆ alkyl substituted with one or more —OSi(R¹³)₃, and R^{1b} is —NR¹³CONR¹¹R¹²; R^{1a} is C₁-C₆ alkyl substituted with one or more —OSi(R¹³)₃, and R^{1b} is —CR¹¹R¹²CN; R^{1a} is C₁-C₆ alkyl substituted with one or more —OSi(R¹³)₃, and R^{1b} is —NR¹¹SO₂R¹³; R^{1a} is C₁-C₆ alkyl substituted with one or more —OSi(R¹³)₃, and R^{1b} is —NR¹¹CONR¹¹R¹²; R^{1a} is C₁-C₆ alkyl substituted with one or more —OSi(R¹³)₃, and R^{1b} is —CN; R^{1a} is C₁-C₆ alkyl substituted with one or more —OSi(R¹³)₃, and R^{1b} is —NR¹¹COR¹²; R^{1a} is C₁-C₆ alkyl substituted with one or more —OSi(R¹³)₃, and R^{1b} is —CR¹¹R¹²NR¹¹R¹²; R^{1a} is —SO₂NR¹¹R¹², and R^{1b} is C₁-C₆ alkyl substituted with one or more hydroxy; R^{1a} is —SO₂NR¹¹R¹², and R^{1b} is —SO₂NR¹¹R¹²; R^{1a} is —SO₂NR¹¹R¹², and R^{1b} is —SO₂R¹³; R^{1a} is —SO₂NR¹¹R¹², and R^{1b} is —CONR¹¹R¹²; R^{1a} is —SO₂NR¹¹R¹², and R^{1b} is —OR¹¹; R^{1a} is —SO₂NR¹¹R¹², and R^{1b} is —COR¹³; R^{1a} is —SO₂NR¹¹R¹², and R^{1b} is —CO₂R¹³; R^{1a} is —SO₂NR¹¹R¹², and R^{1b} is —NR¹³CONR¹¹R¹²; R^{1a} is —SO₂NR¹¹R¹², and R^{1b} is —CR¹¹R¹²CN; R^{1a} is —SO₂NR¹¹R¹², and R^{1b} is —NR¹¹SO₂R¹³; R^{1a} is —SO₂NR¹¹R¹², and R^{1b} is —CR¹¹R¹²NR¹¹R¹²; R^{1a} is —SO₂NR¹¹R¹², and R^{1b} is —CN; R^{1a} is —SO₂NR¹¹R¹², and R^{1b} is —NR¹¹CONR¹¹R¹²; R^{1a} is —SO₂NR¹¹R¹², and R^{1b} is —NR¹¹COR¹²; R^{1a} is —CR¹¹R¹²NR¹¹R¹², and R^{1b} is C₁-C₆ alkyl substituted with one or more hydroxy; R^{1a} is —CR¹¹R¹²NR¹¹R¹², and R^{1b} is —SO₂NR¹¹R¹²; R^{1a} is —CR¹¹R¹²NR¹¹R¹², and R^{1b} is —SO₂R¹³; R^{1a} is —CR¹¹R¹²NR¹¹R¹², and R^{1b} is —CONR¹¹R¹²; R^{1a} is —CR¹¹R¹²NR¹¹R¹², and R^{1b} is —OR¹¹; R^{1a} is —CR¹¹R¹²NR¹¹R¹², and R^{1b} is —COR¹³; R^{1a} is —CR¹¹R¹²NR¹¹R¹², and R^{1b} is —CO₂R¹³; R^{1a} is —CR¹¹R¹²NR¹¹R¹², and R^{1b} is —NR¹³CONR¹¹R¹²; R^{1a} is —CR¹¹R¹²NR¹¹R¹², and R^{1b} is —CR¹¹R¹²CN; R^{1a} is —CR¹¹R¹²NR¹¹R¹², and R^{1b} is —NR¹¹SO₂R¹³; R^{1a} is —CR¹¹R¹²NR¹¹R¹², and R^{1b} is —CR¹¹R¹²NR¹¹R¹²; R^{1a} is —CR¹¹R¹²NR¹¹R¹², and R^{1b} is —CN; R^{1a} is —CR¹¹R¹²NR¹¹R¹², and R^{1b} is —NR¹¹CONR¹¹R¹²; and R^{1a} is —CR¹¹R¹²NR¹¹R¹², and R^{1b} is —NR¹¹COR¹².

In some embodiments of the compound of formula AA,

the substituted ring A is

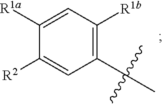

R^{1a} and R^{1b} are one of the following combinations:

In some embodiments, R^{1a} is 2-hydroxy-2-propyl, and R^{1b} is —OMe; In some embodiments, R^{1a} is 2-hydroxy-2-propyl, and R^{1b} is —OH; In some embodiments, R^{1a} is 2-hydroxy-2-propyl, and R^{1b} is —CO₂Me; In some embodiments, R^{1a} is 2-hydroxy-2-propyl, and R^{1b} is hydroxymethyl; In some embodiments, R^{1a} is 2-hydroxy-2-propyl, and R^{1b} is hydroxy ethyl; In some embodiments, R^{1a} is 2-hydroxy-2-propyl, and R^{1b} is 2-hydroxy-2-propyl; In some embodiments, R^{1a} is 2-hydroxy-2-propyl, and R^{1b} is —SO₂NHCH₂CH₂OH; In some embodiments, R^{1a} is 2-hydroxy-2-propyl, and R^{1b} is —SO₂Me; In some embodiments, R^{1a} is 2-hydroxy-2-propyl, and R^{1b} is CONHMe; In some embodiments, R^{1a} is 2-hydroxy-2-propyl, and R^{1b} is cyanomethyl; In some embodiments, R^{1a} is 2-hydroxy-2-propyl, and R^{1b} is —CN; In some embodiments, R^{1a} is 2-hydroxy-2-propyl, and R^{1b} is dimethylaminomethyl; In some embodiments, R^{1a} is dimethylaminomethyl, and R^{1b} is —OMe; In some embodiments, R^{1a} is dimethylaminomethyl, and R^{1b} is —OH; In some embodiments, R^{1a} is dimethylaminomethyl, and R^{1b} is —CO₂Me; In some embodiments, R^{1a} is dimethylaminomethyl, and R^{1b} is hydroxymethyl; In some embodiments, R^{1a} is dimethylaminomethyl, and R^{1b} is hydroxy ethyl; In some embodiments, R^{1a} is dimethylaminomethyl, and R^{1b} is 2-hydroxy-2-propyl; In some embodiments, R^{1a} is dimethylaminomethyl, and R^{1b} is —SO₂NHCH₂CH₂OH; In some embodiments, R^{1a} is dimethylaminomethyl, and R^{1b} is —SO₂Me; In some embodiments, R^{1a} is dimethylaminomethyl, and R^{1b} is CONHMe; In some embodiments, R^{1a} is dimethylaminomethyl, and R^{1b} is cyanomethyl; In some embodiments, R^{1a} is dimethylaminomethyl, and R^{1b} is dimethylaminomethyl; In some embodiments, R^{1a} is dimethylaminomethyl, and R^{1b} is CN In some embodiments, R^{1a} is —SO₂NHMe, and R^{1b} is —OMe; In some embodiments, R^{1a} is —SO₂NHMe, and R^{1b} is —OH; In some embodiments, R^{1a} is —SO₂NHMe, and R^{1b} is —CO₂Me; In some embodiments, R^{1a} is —SO₂NHMe, and R^{1b} is hydroxymethyl; In some embodiments, R^{1a} is —SO₂NHMe, and R^{1b} is hydroxyethyl; In some embodiments, R^{1a} is —SO₂NHMe, and R^{1b} is 2-hydroxy-2-propyl; In some embodiments, R^{1a} is —SO₂NHMe, and R^{1b} is —SO₂NHCH₂CH₂OH; In some embodiments, R^{1a} is —SO₂NHMe, and R^{1b} is —SO₂Me; In some embodiments, R^{1a} is —SO₂NHMe, and R^{1b} is CONHMe; In some embodiments, R^{1a} is —SO₂NHMe, and R^{1b} is dimethylaminomethyl; In some embodiments, R^{1a} is —SO₂NHMe, and R^{1b} is —CN; In some embodiments, R^{1a} is —SO₂NHMe, and R^{1b} is cyanomethyl; and In some embodiments, R^{1a} is C₁-C₄ alkyl substituted with one —OSi(Me)₂tBu, and R^{1b} is —CO₂Me.

In some embodiments of the compound of formula AA,

the substituted ring A is

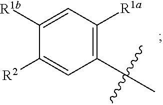

R^{1a} and R^{1b} are one of the following combinations:
R^{1a} is C₁-C₆ alkyl substituted with one or more hydroxy, and R^{1b} is C₁-C₆ alkyl substituted with one or more hydroxy; R^{1a} is C₁-C₆ alkyl substituted with one or more hydroxy, and R^{1b} is —SO₂NR¹¹R¹²; R^{1a} is C₁-C₆ alkyl substituted with one or more hydroxy, and R^{1b} is —SO₂R¹³; R^{1a} is C₁-C₆ alkyl substituted with one or more hydroxy, and R^{1b} is —CONR¹¹R¹²; R^{1a} is C₁-C₆ alkyl substituted with one or more hydroxy, and R^{1b} is —OR¹¹; R^{1a} is C₁-C₆ alkyl substituted with one or more hydroxy, and R^{1b} is —COR¹³; R^{1a} is C₁-C₆ alkyl substituted with one or more hydroxy, and R^{1b} is —CO₂R¹³; R^{1a} is C₁-C₆ alkyl substituted with one or more hydroxy, and R^{1b} is —NR¹³CONR¹¹R¹²; R^{1a} is C₁-C₆ alkyl substituted with one or more hydroxy, and R^{1b} is —CR¹¹R¹²CN; R^{1a} is C₁-C₆ alkyl substituted with one or more hydroxy, and R^{1b} is —NR¹¹SO₂R¹³; R^{1a} is C₁-C₆ alkyl substituted with one or more hydroxy, and R^{1b} is —NR¹¹CONR¹¹R¹²; R^{1a} is C₁-C₆ alkyl substituted with one or more hydroxy, and R^{1b} is —CN; R^{1a} is C₁-C₆ alkyl substituted with one or more hydroxy, and R^{1b} is —NR¹¹COR¹²; R^{1a} is C₁-C₆ alkyl substituted with one or more hydroxy, and R^{1b} is —CR¹¹R¹²NR¹¹R¹²; R^{1a} is C₁-C₆ alkyl substituted with one or more —OSi(R¹³)₃, and R^{1b} is C₁-C₆ alkyl substituted with one or more hydroxy; R^{1a} is C₁-C₆ alkyl substituted with one or more —OSi(R¹³)₃, and R^{1b} is —SO₂NR¹¹R¹²; R^{1a} is C₁-C₆ alkyl substituted with one or more —OSi(R¹³)₃, and R^{1b} is —SO₂R¹³; R^{1a} is C₁-C₆ alkyl substituted with one or more —OSi(R¹³)₃, and R^{1b} is —CONR¹¹R¹²; R^{1a} is C₁-C₆ alkyl substituted with one or more —OSi(R¹³)₃, and R^{1b} is —OR¹¹; R^{1a} is C₁-C₆ alkyl substituted with one or more —OSi(R¹³)₃, and R^{1b} is —COR¹³; R^{1a} is C₁-C₆ alkyl substituted with one or more —OSi(R¹³)₃, and R^{1b} is —CO₂R¹³; R^{1a} is C₁-C₆ alkyl substituted with one or more —OSi(R¹³)₃, and R^{1b} is —NR¹³CONR¹¹R¹²; R^{1a} is C₁-C₆ alkyl substituted with one or more —OSi(R¹³)₃, and R^{1b} is —CR¹¹R¹²CN; R^{1a} is C₁-C₆ alkyl substituted with one or more —OSi(R¹³)₃, and R^{1b} is —NR¹¹SO₂R¹³; R^{1a} is C₁-C₆ alkyl substituted with one or more —OSi(R¹³)₃, and R^{1b} is —NR¹¹CONR¹¹R¹²; R^{1a} is C₁-C₆ alkyl substituted with one or more —OSi(R¹³)₃, and R^{1b} is —CN; R^{1a} is C₁-C₆ alkyl substituted with one or more —OSi(R¹³)₃, and R^{1b} is —NR¹¹COR¹²; R^{1a} is C₁-C₆ alkyl substituted with one or more —OSi(R¹³)₃, and R^{1b} is —CR¹¹R¹²NR¹¹R¹²; R^{1a} is —SO₂NR¹¹R¹², and R^{1b} is C₁-C₆ alkyl substituted with one or more hydroxy; R^{1a} is —SO₂NR¹¹R¹², and R^{1b} is —SO₂NR¹¹R¹²; R^{1a} is —SO₂NR¹¹R¹², and R^{1b} is —SO₂R¹³; R^{1a} is —SO₂NR¹¹R¹², and R^{1b} is —CONR¹¹R¹²; R^{1a} is —SO₂NR¹¹R¹², and R^{1b} is —OR¹¹; R^{1a} is —SO₂NR¹¹R¹², and R^{1b} is —COR¹³; R^{1a} is —SO₂NR¹¹R¹², and R^{1b} is —CO₂R¹³; R^{1a} is —SO₂NR¹¹R¹², and R^{1b} is —NR¹³CONR¹¹R¹²; R^{1a} is —SO₂NR¹¹R¹², and R^{1b} is —CR¹¹R¹²CN; R^{1a} is —SO₂NR¹¹R¹², and R^{1b} is —NR¹¹SO₂R¹³; R^{1a} is —SO₂NR¹¹R¹², and R^{1b} is —CR¹¹R¹²NR¹¹R¹²; R^{1a} is —SO₂NR¹¹R¹², and R^{1b} is —CN; R^{1a} is —SO₂NR¹¹R¹², and R^{1b} is —NR¹¹CONR¹¹R¹²; R^{1a} is —SO₂NR¹¹R¹², and R^{1b} is —NR¹¹COR¹²; R^{1a} is —CR¹¹R¹²NR¹¹R¹², and R^{1b} is C₁-C₆ alkyl substituted with one or more hydroxy; R^{1a} is —CR¹¹R¹²NR¹¹R¹², and R^{1b} is —SO₂NR¹¹R¹²; R^{1a} is —CR¹¹R¹²NR¹¹R¹², and R^{1b} is —SO₂R¹³; R^{1a} is —CR¹¹R¹²NR¹¹R¹², and R^{1b} is —CONR¹¹R¹²; R^{1a} is —CR¹¹R¹²NR¹¹R¹², and R^{1b} is —OR¹¹; R^{1a} is —CR¹¹R¹²NR¹¹R¹², and R^{1b} is —COR¹³; R^{1a} is —CR¹¹R¹²NR¹¹R¹², and R^{1b} is —CO₂R¹³; R^{1a} is —CR¹¹R¹²NR¹¹R¹², and R^{1b} is —NR¹³CONR¹¹R¹²; R^{1a} is —CR¹¹R¹²NR¹¹R¹², and R^{1b} is —CR¹¹R¹²CN; R^{1a} is —CR¹¹R¹²NR¹¹R¹², and R^{1b} is —NR¹¹SO₂R¹³; R^{1a} is —CR¹¹R¹²NR¹¹R¹², and R^{1b} is —CR¹¹R¹²NR¹¹R¹²; R^{1a} is —CR¹¹R¹²NR¹¹R¹², and R^{1b} is —CN; R^{1a} is —CR¹¹R¹²NR¹¹R¹², and R^{1b} is —NR¹¹CONR¹¹R¹²; and R^{1a} is —CR¹¹R¹²NR¹¹R¹², and R^{1b} is —NR¹¹COR¹².

In some embodiments of the compound of formula AA,

the substituted ring A is

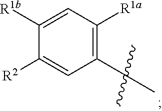

R^{1a} and R^{1b} are one of the following combinations:

In some embodiments, R^{1a} is 2-hydroxy-2-propyl, and R^{1b} is —OMe; In some embodiments, R^{1a} is 2-hydroxy-2-propyl, and R^{1b} is —OH; In some embodiments, R^{1a} is 2-hydroxy-2-propyl, and R^{1b} is —CO₂Me; In some embodiments, R^{1a} is 2-hydroxy-2-propyl, and R^{1b} is hydroxymethyl; In some embodiments, R^{1a} is 2-hydroxy-2-propyl, and R^{1b} is hydroxy ethyl; In some embodiments, R^{1a} is 2-hydroxy-2-propyl, and R^{1b} is 2-hydroxy-2-propyl; In some embodiments, R^{1a} is 2-hydroxy-2-propyl, and R^{1b} is —SO₂NHCH₂CH₂OH; In some embodiments, R^{1a} is 2-hydroxy-2-propyl, and R^{1b} is —SO₂Me; In some embodiments, R^{1a} is 2-hydroxy-2-propyl, and R^{1b} is CONHMe; In some embodiments, R^{1a} is 2-hydroxy-2-propyl, and R^{1b} is cyanomethyl; In some embodiments, R^{1a} is 2-hydroxy-2-propyl, and R^{1b} is —CN; In some embodiments, R^{1a} is 2-hydroxy-2-propyl, and R^{1b} is dimethylaminomethyl; In some embodiments, R^{1a} is dimethylaminomethyl, and R^{1b} is —OMe; In some embodiments, R^{1a} is dimethylaminomethyl, and R^{1b} is —OH; In some embodiments, R^{1a} is dimethylaminomethyl, and R^{1b} is —CO₂Me; In some embodiments, R^{1a} is dimethylaminomethyl, and R^{1b} is hydroxymethyl; In some embodiments, R^{1a} is dimethylaminomethyl, and R^{1b} is hydroxy ethyl; In some embodiments, R^{1a} is dimethylaminomethyl, and R^{1b} is 2-hydroxy-2-propyl; In some embodiments, R^{1a} is dimethylaminomethyl, and R^{1b} is —SO₂NHCH₂CH₂OH; In some embodiments, R^{1a} is dimethylaminomethyl, and R^{1b} is —SO₂Me; In some embodiments, R^{1a} is dimethylaminomethyl, and R^{1b} is CONHMe; In some embodiments, R^{1a} is dimethylaminomethyl, and R^{1b} is cyanomethyl; In some embodiments, R^{1a} is dimethylaminomethyl, and R^{1b} is dimethylaminomethyl; In some embodiments, R^{1a} is dimethylaminomethyl, and R^{1b} is CN In some embodiments, R^{1a} is —SO₂NHMe, and R^{1b} is —OMe; In some embodiments, R^{1a} is —SO₂NHMe, and R^{1b} is —OH; In some embodiments, R^{1a} is —SO₂NHMe, and R^{1b} is —CO₂Me; In some embodiments, R^{1a} is —SO₂NHMe, and R^{1b} is hydroxymethyl; In some embodiments, R^{1a} is —SO₂NHMe, and R^{1b} is hydroxyethyl; In some embodiments, R^{1a} is —SO₂NHMe, and R^{1b} is 2-hydroxy-2-propyl; In some embodiments, R^{1a} is —SO₂NHMe, and R^{1b} is —SO₂NHCH₂CH₂OH; In some embodiments, R^{1a} is —SO₂NHMe, and R^{1b} is —SO₂Me; In some embodiments, R^{1a} is —SO₂NHMe, and R^{1b} is CONHMe; In some embodiments, R^{1a} is —SO₂NHMe, and R^{1b} is dimethylaminomethyl; In some embodiments, R^{1a} is —SO₂NHMe, and R^{1b} is —CN; In some embodiments, R^{1a} is —SO₂NHMe, and R^{1b} is cyanomethyl; and In some embodiments, R^{1a} is C₁-C₄ alkyl substituted with one —OSi(Me)₂tBu, and R^{1b} is —CO₂Me.

In some embodiments of the compound of formula AA,

the substituted ring A is

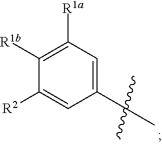

R^{1a} and R^{1b} are one of the following combinations:
R^{1a} is C₁-C₆ alkyl substituted with one or more hydroxy, and R^{1b} is C₁-C₆ alkyl substituted with one or more hydroxy; R^{1a} is C₁-C₆ alkyl substituted with one or more hydroxy, and R^{1b} is —SO₂NR¹¹R¹²; R^{1a} is C₁-C₆ alkyl substituted with one or more hydroxy, and R^{1b} is —SO₂R¹³; R^{1a} is C₁-C₆ alkyl substituted with one or more hydroxy, and R^{1b} is —CONR¹¹R¹²; R^{1a} is C₁-C₆ alkyl substituted with one or more hydroxy, and R^{1b} is —OR¹¹; R^{1a} is C₁-C₆ alkyl substituted with one or more hydroxy, and R^{1b} is —COR¹³; R^{1a} is C₁-C₆ alkyl substituted with one or more hydroxy, and R^{1b} is —CO₂R¹³; R^{1a} is C₁-C₆ alkyl substituted with one or more hydroxy, and R^{1b} is —NR¹³CONR¹¹R¹²; R^{1a} is C₁-C₆ alkyl substituted with one or more hydroxy, and R^{1b} is —CR¹¹R¹²CN; R^{1a} is C₁-C₆ alkyl substituted with one or more hydroxy, and R^{1b} is —NR¹¹SO₂R¹³; R^{1a} is C₁-C₆ alkyl substituted with one or more hydroxy, and R^{1b} is —NR¹¹CONR¹¹R¹²; R^{1a} is C₁-C₆ alkyl substituted with one or more hydroxy, and R^{1b} is —CN; R^{1a} is C₁-C₆ alkyl substituted with one or more hydroxy, and R^{1b} is —NR¹¹COR¹²; R^{1a} is C₁-C₆ alkyl substituted with one or more hydroxy, and R^{1b} is —CR¹¹R¹²NR¹¹R¹²; R^{1a} is C₁-C₆ alkyl substituted with one or more —OSi(R¹³)₃, and R^{1b} is C₁-C₆ alkyl substituted with one or more hydroxy; R^{1a} is C₁-C₆ alkyl substituted with one or more —OSi(R¹³)₃, and R^{1b} is —SO₂NR¹¹R¹²; R^{1a} is C₁-C₆ alkyl substituted with one or more —OSi(R¹³)₃, and R^{1b} is —SO₂R¹³; R^{1a} is C₁-C₆ alkyl substituted with one or more —OSi(R¹³)₃, and R^{1b} is —CONR¹¹R¹²; R^{1a} is C₁-C₆ alkyl substituted with one or more —OSi(R¹³)₃, and R^{1b} is —OR¹¹; R^{1a} is C₁-C₆ alkyl substituted with one or more —OSi(R¹³)₃, and R^{1b} is —COR¹³; R^{1a} is C₁-C₆ alkyl substituted with one or more —OSi(R¹³)₃, and R^{1b} is —CO₂R¹³; R^{1a} is C₁-C₆ alkyl substituted with one or more —OSi(R¹³)₃, and R^{1b} is —NR¹³CONR¹¹R¹²; R^{1a} is C₁-C₆ alkyl substituted with one or more —OSi(R¹³)₃, and R^{1b} is —CR¹¹R¹²CN; R^{1a} is C₁-C₆ alkyl substituted with one or more —OSi(R¹³)₃, and R^{1b} is —NR¹¹SO₂R¹³; R^{1a} is C₁-C₆ alkyl substituted with one or more —OSi(R¹³)₃, and R^{1b} is —NR¹¹CONR¹¹R¹²; R^{1a} is C₁-C₆ alkyl substituted with one or more —OSi(R¹³)₃, and R^{1b} is —CN; R^{1a} is C₁-C₆ alkyl substituted with one or more —OSi(R¹³)₃, and R^{1b} is —NR¹¹COR¹²; R^{1a} is C₁-C₆ alkyl substituted with one or more —OSi(R¹³)₃, and R^{1b} is —CR¹¹R¹²NR¹¹R¹²; R^{1a} is —SO₂NR¹¹R¹², and R^{1b} is C₁-C₆ alkyl substituted with one or more hydroxy; R^{1a} is —SO₂NR¹¹R¹², and R^{1b} is —SO₂NR¹¹R¹²; R^{1a} is —SO₂NR¹¹R¹², and R^{1b} is —SO₂R¹³; R^{1a} is —SO₂NR¹¹R¹², and R^{1b} is —CONR¹¹R¹²; R^{1a} is —SO₂NR¹¹R¹², and R^{1b} is —OR¹¹; R^{1a} is —SO₂NR¹¹R¹², and R^{1b} is —COR¹³; R^{1a} is —SO₂NR¹¹R¹², and R^{1b} is —CO₂R¹³; R^{1a} is —SO₂NR¹¹R¹², and R^{1b} is —NR¹³CONR¹¹R¹²; R^{1a} is —SO₂NR¹¹R¹², and R^{1b} is —CR¹¹R¹²CN; R^{1a} is —SO₂NR¹¹R¹², and R^{1b} is —NR¹¹SO₂R¹³; R^{1a} is —SO₂NR¹¹R¹², and R^{1b} is —CR¹¹R¹²NR¹¹R¹²; R^{1a} is —SO₂NR¹¹R¹², and R^{1b} is —CN; R^{1a} is —SO₂NR¹¹R¹², and R^{1b} is —NR¹¹CONR¹¹R¹²; R^{1a} is —SO₂NR¹¹R¹², and R^{1b} is —NR¹¹COR¹²; R^{1a} is —CR¹¹R¹²NR¹¹R¹², and R^{1b} is C₁-C₆ alkyl substituted with one or more hydroxy; R^{1a} is —CR¹¹R¹²NR¹¹R¹², and R^{1b} is —SO₂NR¹¹R¹²; R^{1a} is —CR¹¹R¹²NR¹¹R¹², and R^{1b} is —SO₂R¹³; R^{1a} is —CR¹¹R¹²NR¹¹R¹², and R^{1b} is —CONR¹¹R¹²; R^{1a} is —CR¹¹R¹²NR¹¹R¹², and R^{1b} is —OR¹¹; R^{1a} is —CR¹¹R¹²NR¹¹R¹², and R^{1b} is —COR¹³; R^{1a} is —CR¹¹R¹²NR¹¹R¹², and R^{1b} is —CO₂R¹³; R^{1a} is —CR¹¹R¹²NR¹¹R¹², and R^{1b} is —NR¹³CONR¹¹R¹²; R^{1a} is —CR¹¹R¹²NR¹¹R¹², and R^{1b} is —CR¹¹R¹²CN; R^{1a} is —CR¹¹R¹²NR¹¹R¹², and R^{1b} is —NR¹¹SO₂R¹³; R^{1a} is —CR¹¹R¹²NR¹¹R¹², and R^{1b} is —CR¹¹R¹²NR¹¹R¹²; R^{1a} is —CR¹¹R¹²NR¹¹R¹², and R^{1b} is —CN; R^{1a} is —CR¹¹R¹²NR¹¹R¹², and R^{1b} is —NR¹¹CONR¹¹R¹²; and R^{1a} is —CR¹¹R¹²NR¹¹R¹², and R^{1b} is —NR¹¹COR¹².

In some embodiments of the compound of formula AA,

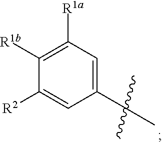

the substituted ring A is
R^{1a} and R^{1b} are one of the following combinations:

In some embodiments, R^{1a} is 2-hydroxy-2-propyl, and R^{1b} is —OMe; In some embodiments, R^{1a} is 2-hydroxy-2-propyl, and R^{1b} is —OH; In some embodiments, R^{1a} is 2-hydroxy-2-propyl, and R^{1b} is —CO₂Me; In some embodiments, R^{1a} is 2-hydroxy-2-propyl, and R^{1b} is hydroxymethyl; In some embodiments, R^{1a} is 2-hydroxy-2-propyl, and R^{1b} is hydroxy ethyl; In some embodiments, R^{1a} is 2-hydroxy-2-propyl, and R^{1b} is 2-hydroxy-2-propyl; In some embodiments, R^{1a} is 2-hydroxy-2-propyl, and R^{1b} is —SO₂NHCH₂CH₂OH; In some embodiments, R^{1a} is 2-hydroxy-2-propyl, and R^{1b} is —SO₂Me; In some embodiments, R^{1a} is 2-hydroxy-2-propyl, and R^{1b} is CONHMe; In some embodiments, R^{1a} is 2-hydroxy-2-propyl, and R^{1b} is cyanomethyl; In some embodiments, R^{1a} is 2-hydroxy-2-propyl, and R^{1b} is —CN; In some embodiments, R^{1a} is 2-hydroxy-2-propyl, and R^{1b} is dimethylaminomethyl; In some embodiments, R^{1a} is dimethylaminomethyl, and R^{1b} is —OMe; In some embodiments, R^{1a} is dimethylaminomethyl, and R^{1b} is —OH; In some embodiments, R^{1a} is dimethylaminomethyl, and R^{1b} is —CO₂Me; In some embodiments, R^{1a} is dimethylaminomethyl, and R^{1b} is hydroxymethyl; In some embodiments, R^{1a} is dimethylaminomethyl, and R^{1b} is hydroxy ethyl; In some embodiments, R^{1a} is dimethylaminomethyl, and R^{1b} is 2-hydroxy-2-propyl; In some embodiments, R^{1a} is dimethylaminomethyl, and R^{1b} is —SO₂NHCH₂CH₂OH; In some embodiments, R^{1a} is dimethylaminomethyl, and R^{1b} is —SO₂Me; In some embodiments, R^{1a} is dimethylaminomethyl, and R^{1b} is CONHMe; In some embodiments, R^{1a} is dimethylaminomethyl, and R^{1b} is cyanomethyl; In some embodiments, R^{1a} is dimethylaminomethyl, and R^{1b} is dimethylaminomethyl; In some embodiments, R^{1a} is dimethylaminomethyl, and R^{1b} is CN In some embodiments, R^{1a} is —SO₂NHMe, and R^{1b} is —OMe; In some embodiments, R^{1a} is —SO₂NHMe, and R^{1b} is —OH; In some embodiments, R^{1a} is —SO₂NHMe, and R^{1b} is —CO₂Me; In some embodiments, R^{1a} is —SO₂NHMe, and R^{1b} is hydroxymethyl; In some embodiments, R^{1a} is —SO₂NHMe, and R^{1b} is hydroxyethyl; In some embodiments, R^{1a} is —SO₂NHMe, and R^{1b} is 2-hydroxy-2-propyl; In some embodiments, R^{1a} is —SO₂NHMe, and R^{1b} is —SO₂NHCH₂CH₂OH; In some embodiments, R^{1a} is —SO₂NHMe, and R^{1b} is —SO₂Me; In some embodiments, R^{1a} is —SO₂NHMe, and R^{1b} is CONHMe; In some embodiments, R^{1a} is —SO₂NHMe, and R^{1b} is dimethylaminomethyl; In some embodiments, R^{1a} is —SO₂NHMe, and R^{1b} is —CN; In some embodiments, R^{1a} is —SO₂NHMe, and R^{1b} is cyanomethyl; and In some embodiments, R^{1a} is C₁-C₄ alkyl substituted with one —OSi(Me)₂tBu, and R^{1b} is —CO₂Me.

In some embodiments of the compound of formula AA,

the substituted ring A is

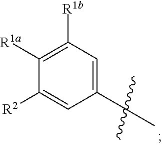

R^{1a} and R^{1b} are one of the following combinations:
R^{1a} is C₁-C₆ alkyl substituted with one or more hydroxy, and R^{1b} is C₁-C₆ alkyl substituted with one or more hydroxy; R^{1a} is C₁-C₆ alkyl substituted with one or more hydroxy, and R^{1b} is —SO₂NR¹¹R¹²; R^{1a} is C₁-C₆ alkyl substituted with one or more hydroxy, and R^{1b} is —SO₂R¹³; R^{1a} is C₁-C₆ alkyl substituted with one or more hydroxy, and R^{1b} is —CONR¹¹R¹²; R^{1a} is C₁-C₆ alkyl substituted with one or more hydroxy, and R^{1b} is —OR¹¹; R^{1a} is C₁-C₆ alkyl substituted with one or more hydroxy, and R^{1b} is —COR¹³; R^{1a} is C₁-C₆ alkyl substituted with one or more hydroxy, and R^{1b} is —CO₂R¹³; R^{1a} is C₁-C₆ alkyl substituted with one or more hydroxy, and R^{1b} is —NR¹³CONR¹¹R¹²; R^{1a} is C₁-C₆ alkyl substituted with one or more hydroxy, and R^{1b} is —CR¹¹R¹²CN; R^{1a} is C₁-C₆ alkyl substituted with one or more hydroxy, and R^{1b} is —NR¹¹SO₂R¹³; R^{1a} is C₁-C₆ alkyl substituted with one or more hydroxy, and R^{1b} is —NR¹¹CONR¹¹R¹²; R^{1a} is C₁-C₆ alkyl substituted with one or more hydroxy, and R^{1b} is —CN; R^{1a} is C₁-C₆ alkyl substituted with one or more hydroxy, and R^{1b} is —NR¹¹COR¹²; R^{1a} is C₁-C₆ alkyl substituted with one or more hydroxy, and R^{1b} is —CR¹¹R¹²NR¹¹R¹²; R^{1a} is C₁-C₆ alkyl substituted with one or more —OSi(R¹³)₃, and R^{1b} is C₁-C₆ alkyl substituted with one or more hydroxy; R^{1a} is C₁-C₆ alkyl substituted with one or more —OSi(R¹³)₃, and R^{1b} is —SO₂NR¹¹R¹²; R^{1a} is C₁-C₆ alkyl substituted with one or more —OSi(R¹³)₃, and R^{1b} is —SO₂R¹³; R^{1a} is C₁-C₆ alkyl substituted with one or more —OSi(R¹³)₃, and R^{1b} is —CONR¹¹R¹²; R^{1a} is C₁-C₆ alkyl substituted with one or more —OSi(R¹³)₃, and R^{1b} is —OR¹¹; R^{1a} is C₁-C₆ alkyl substituted with one or more —OSi(R¹³)₃, and R^{1b} is —COR¹³; R^{1a} is C₁-C₆ alkyl substituted with one or more —OSi(R¹³)₃, and R^{1b} is —CO₂R¹³; R^{1a} is C₁-C₆ alkyl substituted with one or more —OSi(R¹³)₃, and R^{1b} is —NR¹³CONR¹¹R¹²; R^{1a} is C₁-C₆ alkyl substituted with one or more —OSi(R¹³)₃, and R^{1b} is —CR¹¹R¹²CN; R^{1a} is C₁-C₆ alkyl substituted with one or more —OSi(R¹³)₃, and R^{1b} is —NR¹¹SO₂R¹³; R^{1a} is C₁-C₆ alkyl substituted with one or more —OSi(R¹³)₃, and R^{1b} is —NR¹¹CONR¹¹R¹²; R^{1a} is C₁-C₆ alkyl substituted with one or more —OSi(R¹³)₃, and R^{1b} is —CN; R^{1a} is C₁-C₆ alkyl substituted with one or more —OSi(R¹³)₃, and R^{1b} is —NR¹¹COR¹²; R^{1a} is C₁-C₆ alkyl substituted with one or more —OSi(R¹³)₃, and R^{1b} is —CR¹¹R¹²NR¹¹R¹²; R^{1a} is —SO₂NR¹¹R¹², and R^{1b} is C₁-C₆ alkyl substituted with one or more hydroxy; R^{1a} is —SO₂NR¹¹R¹², and R^{1b} is —SO₂NR¹¹R¹²; R^{1a} is —SO₂NR¹¹R¹², and R^{1b} is —SO₂R¹³; R^{1a} is —SO₂NR¹¹R¹², and R^{1b} is —CONR¹¹R¹²; R^{1a} is —SO₂NR¹¹R¹², and R^{1b} is —OR¹¹; R^{1a} is —SO₂NR¹¹R¹², and R^{1b} is —COR¹³; R^{1a} is —SO₂NR¹¹R¹², and R^{1b} is —CO₂R¹³; R^{1a} is —SO₂NR¹¹R¹², and R^{1b} is —NR¹³CONR¹¹R¹²; R^{1a} is —SO₂NR¹¹R¹², and R^{1b} is —CR¹¹R¹²CN; R^{1a} is —SO₂NR¹¹R¹², and R^{1b} is —NR¹¹SO₂R¹³; R^{1a} is —SO₂NR¹¹R¹², and R^{1b} is —CR¹¹R¹²NR¹¹R¹²; R^{1a} is —SO₂NR¹¹R¹², and R^{1b} is —CN; R^{1a} is —SO₂NR¹¹R¹², and R^{1b} is —NR¹¹CONR¹¹R¹²; R^{1a} is —SO₂NR¹¹R¹², and R^{1b} is —NR¹¹COR¹²; R^{1a} is —CR¹¹R¹²NR¹¹R¹², and R^{1b} is C₁-C₆ alkyl substituted with one or more hydroxy; R^{1a} is —CR¹¹R¹²NR¹¹R¹², and R^{1b} is —SO₂NR¹¹R¹²; R^{1a} is —CR¹¹R¹²NR¹¹R¹², and R^{1b} is —SO₂R¹³; R^{1a} is —CR¹¹R¹²NR¹¹R¹², and R^{1b} is —CONR¹¹R¹²; R^{1a} is —CR¹¹R¹²NR¹¹R¹², and R^{1b} is —OR¹¹; R^{1a} is —CR¹¹R¹²NR¹¹R¹², and R^{1b} is —COR¹³; R^{1a} is —CR¹¹R¹²NR¹¹R¹², and R^{1b} is —CO₂R¹³; R^{1a} is —CR¹¹R¹²NR¹¹R¹², and R^{1b} is —NR¹³CONR¹¹R¹²; R^{1a} is —CR¹¹R¹²NR¹¹R¹², and R^{1b} is —CR¹¹R¹²CN; R^{1a} is —CR¹¹R¹²NR¹¹R¹², and R^{1b} is —NR¹¹SO₂R¹³; R^{1a} is —CR¹¹R¹²NR¹¹R¹², and R^{1b} is —CR¹¹R¹²NR¹¹R¹²; R^{1a} is —CR¹¹R¹²NR¹¹R¹², and R^{1b} is —CN; R^{1a} is —CR¹¹R¹²NR¹¹R¹², and R^{1b} is —NR¹¹CONR¹¹R¹²; and R^{1a} is —CR¹¹R¹²NR¹¹R¹², and R^{1b} is —NR¹¹COR¹².

In some embodiments of the compound of formula AA,

the substituted ring A is

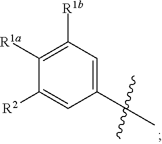

R^{1a} and R^{1b} are one of the following combinations:

In some embodiments, R^{1a} is 2-hydroxy-2-propyl, and R^{1b} is —OMe; In some embodiments, R^{1a} is 2-hydroxy-2-propyl, and R^{1b} is —OH; In some embodiments, R^{1a} is 2-hydroxy-2-propyl, and R^{1b} is —CO₂Me; In some embodiments, R^{1a} is 2-hydroxy-2-propyl, and R^{1b} is hydroxymethyl; In some embodiments, R^{1a} is 2-hydroxy-2-propyl, and R^{1b} is hydroxy ethyl; In some embodiments, R^{1a} is 2-hydroxy-2-propyl, and R^{1b} is 2-hydroxy-2-propyl; In some embodiments, R^{1a} is 2-hydroxy-2-propyl, and R^{1b} is —SO₂NHCH₂CH₂OH; In some embodiments, R^{1a} is 2-hydroxy-2-propyl, and R^{1b} is —SO₂Me; In some embodiments, R^{1a} is 2-hydroxy-2-propyl, and R^{1b} is CONHMe; In some embodiments, R^{1a} is 2-hydroxy-2-propyl, and R^{1b} is cyanomethyl; In some embodiments, R^{1a} is 2-hydroxy-2-propyl, and R^{1b} is —CN; In some embodiments, R^{1a} is 2-hydroxy-2-propyl, and R^{1b} is dimethylaminomethyl; In some embodiments, R^{1a} is dimethylaminomethyl, and R^{1b} is —OMe; In some embodiments, R^{1a} is dimethylaminomethyl, and R^{1b} is —OH; In some embodiments, R^{1a} is dimethylaminomethyl, and R^{1b} is —CO₂Me; In some embodiments, R^{1a} is dimethylaminomethyl, and R^{1b} is hydroxymethyl; In some embodiments, R^{1a} is dimethylaminomethyl, and R^{1b} is hydroxy ethyl; In some embodiments, R^{1a} is dimethylaminomethyl, and R^{1b} is 2-hydroxy-2-propyl; In some embodiments, R^{1a} is dimethylaminomethyl, and R^{1b} is —SO₂NHCH₂CH₂OH; In some embodiments, R^{1a} is dimethylaminomethyl, and R^{1b} is —SO₂Me; In some embodiments, R^{1a} is dimethylaminomethyl, and R^{1b} is CONHMe; In some embodiments, R^{1a} is dimethylaminomethyl, and R^{1b} is cyanomethyl; In some embodiments, R^{1a} is dimethylaminomethyl, and R^{1b} is dimethylaminomethyl; In some embodiments, R^{1a} is dimethylaminomethyl, and R^{1b} is CN In some embodiments, R^{1a} is —SO₂NHMe, and R^{1b} is —OMe; In some embodiments, R^{1a} is —SO₂NHMe, and R^{1b} is —OH; In some embodiments, R^{1a} is —SO₂NHMe, and R^{1b} is —CO₂Me; In some embodiments, R^{1a} is —SO₂NHMe, and R^{1b} is hydroxymethyl; In some embodiments, R^{1a} is —SO₂NHMe, and R^{1b} is hydroxyethyl; In some embodiments, R^{1a} is —SO₂NHMe, and R^{1b} is 2-hydroxy-2-propyl; In some embodiments, R^{1a} is —SO₂NHMe, and R^{1b} is —SO₂NHCH₂CH₂OH; In some embodiments, R^{1a} is —SO₂NHMe, and R^{1b} is —SO₂Me; In some embodiments, R^{1a} is —SO₂NHMe, and R^{1b} is CONHMe; In some embodiments, R^{1a} is —SO₂NHMe, and R^{1b} is dimethylaminomethyl; In some embodiments, R^{1a} is —SO₂NHMe, and R^{1b} is —CN; In some embodiments, R^{1a} is —SO₂NHMe, and R^{1b} is cyanomethyl; and In some embodiments, R^{1a} is C₁-C₄ alkyl substituted with one —OSi(Me)₂tBu, and R^{1b} is —CO₂Me.

In some embodiments of the compound of formula AA,

the substituted ring A is

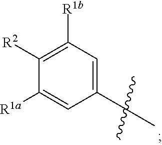

R^{1a} and R^{1b} are one of the following combinations:
R^{1a} is C₁-C₆ alkyl substituted with one or more hydroxy, and R^{1b} is C₁-C₆ alkyl substituted with one or more hydroxy; R^{1a} is C₁-C₆ alkyl substituted with one or more hydroxy, and R^{1b} is —SO₂NR¹¹R¹²; R^{1a} is C₁-C₆ alkyl substituted with one or more hydroxy, and R^{1b} is —SO₂R¹³; R^{1a} is C₁-C₆ alkyl substituted with one or more hydroxy, and R^{1b} is —CONR¹¹R¹²; R^{1a} is C₁-C₆ alkyl substituted with one or more hydroxy, and R^{1b} is —OR¹¹; R^{1a} is C₁-C₆ alkyl substituted with one or more hydroxy, and R^{1b} is —COR¹³; R^{1a} is C₁-C₆ alkyl substituted with one or more hydroxy, and R^{1b} is —CO₂R¹³; R^{1a} is C₁-C₆ alkyl substituted with one or more hydroxy, and R^{1b} is —NR¹³CONR¹¹R¹²; R^{1a} is C₁-C₆ alkyl substituted with one or more hydroxy, and R^{1b} is —CR¹¹R¹²CN; R^{1a} is C₁-C₆ alkyl substituted with one or more hydroxy, and R^{1b} is —NR¹¹SO₂R¹³; R^{1a} is C₁-C₆ alkyl substituted with one or more hydroxy, and R^{1b} is —NR¹¹CONR¹¹R¹²; R^{1a} is C₁-C₆ alkyl substituted with one or more hydroxy, and R^{1b} is —CN; R^{1a} is C₁-C₆ alkyl substituted with one or more hydroxy, and R^{1b} is —NR¹¹COR¹²; R^{1a} is C₁-C₆ alkyl substituted with one or more hydroxy, and R^{1b} is —CR¹¹R¹²NR¹¹R¹²; R^{1a} is C₁-C₆ alkyl substituted with one or more —OSi(R¹³)₃, and R^{1b} is C₁-C₆ alkyl substituted with one or more hydroxy; R^{1a} is C₁-C₆ alkyl substituted with one or more —OSi(R¹³)₃, and R^{1b} is —SO₂NR¹¹R¹²; R^{1a} is C₁-C₆ alkyl substituted with one or more —OSi(R¹³)₃, and R^{1b} is —SO₂R¹³; R^{1a} is C₁-C₆ alkyl substituted with one or more —OSi(R¹³)₃, and R^{1b} is —CONR¹¹R¹²; R^{1a} is C₁-C₆ alkyl substituted with one or more —OSi(R¹³)₃, and R^{1b} is —OR¹¹; R^{1a} is C₁-C₆ alkyl substituted with one or more —OSi(R¹³)₃, and R^{1b} is —COR¹³; R^{1a} is C₁-C₆ alkyl substituted with one or more —OSi(R¹³)₃, and R^{1b} is —CO₂R¹³; R^{1a} is C₁-C₆ alkyl substituted with one or more —OSi(R¹³)₃, and R^{1b} is —NR¹³CONR¹¹R¹²; R^{1a} is C₁-C₆ alkyl substituted with one or more —OSi(R¹³)₃, and R^{1b} is —CR¹¹R¹²CN; R^{1a} is C₁-C₆ alkyl substituted with one or more —OSi(R¹³)₃, and R^{1b} is —NR¹¹SO₂R¹³; R^{1a} is C₁-C₆ alkyl substituted with one or more —OSi(R¹³)₃, and R^{1b} is —NR¹¹CONR¹¹R¹²; R^{1a} is C₁-C₆ alkyl substituted with one or more —OSi(R¹³)₃, and R^{1b} is —CN; R^{1a} is C₁-C₆ alkyl substituted with one or more —OSi(R¹³)₃, and R^{1b} is —NR¹¹COR¹²; R^{1a} is C₁-C₆ alkyl substituted with one or more —OSi(R¹³)₃, and R^{1b} is —CR¹¹R¹²NR¹¹R¹²; R^{1a} is —SO₂NR¹¹R¹², and R^{1b} is C₁-C₆ alkyl substituted with one or more hydroxy; R^{1a} is —SO₂NR¹¹R¹², and R^{1b} is —SO₂NR¹¹R¹²; R^{1a} is —SO₂NR¹¹R¹², and R^{1b} is —SO₂R¹³; R^{1a} is —SO₂NR¹¹R¹², and R^{1b} is —CONR¹¹R¹²; R^{1a} is —SO₂NR¹¹R¹², and R^{1b} is —OR¹¹; R^{1a} is —SO₂NR¹¹R¹², and R^{1b} is —COR¹³; R^{1a} is —SO₂NR¹¹R¹², and R^{1b} is —CO₂R¹³; R^{1a} is —SO₂NR¹¹R¹², and R^{1b} is —NR¹³CONR¹¹R¹²; R^{1a} is —SO₂NR¹¹R¹², and R^{1b} is —CR¹¹R¹²CN; R^{1a} is —SO₂NR¹¹R¹², and R^{1b} is —NR¹¹SO₂R¹³; R^{1a} is —SO₂NR¹¹R¹², and R^{1b} is —CR¹¹R¹²NR¹¹R¹²; R^{1a} is —SO₂NR¹¹R¹², and R^{1b} is —CN; R^{1a} is —SO₂NR¹¹R¹², and R^{1b} is —NR¹¹CONR¹¹R¹²; R^{1a} is —SO₂NR¹¹R¹², and R^{1b} is —NR¹¹COR¹²; R^{1a} is —CR¹¹R¹²NR¹¹R¹², and R^{1b} is C₁-C₆ alkyl substituted with one or more hydroxy; R^{1a} is —CR¹¹R¹²NR¹¹R¹², and R^{1b} is —SO₂NR¹¹R¹²; R^{1a} is —CR¹¹R¹²NR¹¹R¹², and R^{1b} is —SO₂R¹³; R^{1a} is —CR¹¹R¹²NR¹¹R¹², and R^{1b} is —CONR¹¹R¹²; R^{1a} is —CR¹¹R¹²NR¹¹R¹², and R^{1b} is —OR¹¹; R^{1a} is —CR¹¹R¹²NR¹¹R¹², and R^{1b} is —COR¹³; R^{1a} is —CR¹¹R¹²NR¹¹R¹², and R^{1b} is —CO₂R¹³; R^{1a} is —CR¹¹R¹²NR¹¹R¹², and R^{1b} is —NR¹³CONR¹¹R¹²; R^{1a} is —CR¹¹R¹²NR¹¹R¹², and R^{1b} is —CR¹¹R¹²CN; R^{1a} is —CR¹¹R¹²NR¹¹R¹², and R^{1b} is —NR¹¹SO₂R¹³; R^{1a} is —CR¹¹R¹²NR¹¹R¹², and R^{1b} is —CR¹¹R¹²NR¹¹R¹²; R^{1a} is —CR¹¹R¹²NR¹¹R¹², and R^{1b} is —CN; R^{1a} is —CR¹¹R¹²NR¹¹R¹², and R^{1b} is —NR¹¹CONR¹¹R¹²; and R^{1a} is —CR¹¹R¹²NR¹¹R¹², and R^{1b} is —NR¹¹COR¹².

In some embodiments of the compound of formula AA,

the substituted ring A is

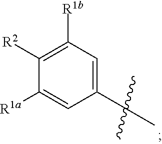

R^{1a} and R^{1b} are one of the following combinations:

In some embodiments, R^{1a} is 2-hydroxy-2-propyl, and R^{1b} is —OMe; In some embodiments, R^{1a} is 2-hydroxy-2-propyl, and R^{1b} is —OH; In some embodiments, R^{1a} is 2-hydroxy-2-propyl, and R^{1b} is —CO₂Me; In some embodiments, R^{1a} is 2-hydroxy-2-propyl, and R^{1b} is hydroxymethyl; In some embodiments, R^{1a} is 2-hydroxy-2-propyl, and R^{1b} is hydroxy ethyl; In some embodiments, R^{1a} is 2-hydroxy-2-propyl, and R^{1b} is 2-hydroxy-2-propyl; In some embodiments, R^{1a} is 2-hydroxy-2-propyl, and R^{1b} is —SO₂NHCH₂CH₂OH; In some embodiments, R^{1a} is 2-hydroxy-2-propyl, and R^{1b} is —SO₂Me; In some embodiments, R^{1a} is 2-hydroxy-2-propyl, and R^{1b} is CONHMe; In some embodiments, R^{1a} is 2-hydroxy-2-propyl, and R^{1b} is cyanomethyl; In some embodiments, R^{1a} is 2-hydroxy-2-propyl, and R^{1b} is —CN; In some embodiments, R^{1a} is 2-hydroxy-2-propyl, and R^{1b} is dimethylaminomethyl; In some embodiments, R^{1a} is dimethylaminomethyl, and R^{1b} is —OMe; In some embodiments, R^{1a} is dimethylaminomethyl, and R^{1b} is —OH; In some embodiments, R^{1a} is dimethylaminomethyl, and R^{1b} is —CO₂Me; In some embodiments, R^{1a} is dimethylaminomethyl, and R^{1b} is hydroxymethyl; In some embodiments, R^{1a} is dimethylaminomethyl, and R^{1b} is hydroxy ethyl; In some embodiments, R^{1a} is dimethylaminomethyl, and R^{1b} is 2-hydroxy-2-propyl; In some embodiments, R^{1a} is dimethylaminomethyl, and R^{1b} is —SO₂NHCH₂CH₂OH; In some embodiments, R^{1a} is dimethylaminomethyl, and R^{1b} is —SO₂Me; In some embodiments, R^{1a} is dimethylaminomethyl, and R^{1b} is CONHMe; In some embodiments, R^{1a} is dimethylaminomethyl, and R^{1b} is cyanomethyl; In some embodiments, R^{1a} is dimethylaminomethyl, and R^{1b} is dimethylaminomethyl; In some embodiments, R^{1a} is dimethylaminomethyl, and R^{1b} is CN In some embodiments, R^{1a} is —SO₂NHMe, and R^{1b} is —OMe; In some embodiments, R^{1a} is —SO₂NHMe, and R^{1b} is —OH; In some embodiments, R^{1a} is —SO₂NHMe, and R^{1b} is —CO₂Me; In some embodiments, R^{1a} is —SO₂NHMe, and R^{1b} is hydroxymethyl; In some embodiments, R^{1a} is —SO₂NHMe, and R^{1b} is hydroxyethyl; In some embodiments, R^{1a} is —SO₂NHMe, and R^{1b} is 2-hydroxy-2-propyl; In some embodiments, R^{1a} is —SO₂NHMe, and R^{1b} is —SO₂NHCH₂CH₂OH; In some embodiments, R^{1a} is —SO₂NHMe, and R^{1b} is —SO₂Me; In some embodiments, R^{1a} is —SO₂NHMe, and R^{1b} is CONHMe; In some embodiments, R^{1a} is —SO₂NHMe, and R^{1b} is dimethylaminomethyl; In some embodiments, R^{1a} is —SO₂NHMe, and R^{1b} is —CN; In some embodiments, R^{1a} is —SO₂NHMe, and R^{1b} is cyanomethyl; and In some embodiments, R^{1a} is C₁-C₄ alkyl substituted with one —OSi(Me)₂tBu, and R^{1b} is —CO₂Me.

In some embodiments of the compound of formula AA,

the substituted ring A is

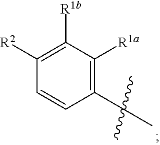

R^{1a} and R^{1b} are one of the following combinations:
R^{1a} is C₁-C₆ alkyl substituted with one or more hydroxy, and R^{1b} is C₁-C₆ alkyl substituted with one or more hydroxy; R^{1a} is C₁-C₆ alkyl substituted with one or more hydroxy, and R^{1b} is —SO₂NR¹¹R¹²; R^{1a} is C₁-C₆ alkyl substituted with one or more hydroxy, and R^{1b} is —SO₂R¹³; R^{1a} is C₁-C₆ alkyl substituted with one or more hydroxy, and R^{1b} is —CONR¹¹R¹²; R^{1a} is C₁-C₆ alkyl substituted with one or more hydroxy, and R^{1b} is —OR¹¹; R^{1a} is C₁-C₆ alkyl substituted with one or more hydroxy, and R^{1b} is —COR¹³; R^{1a} is C₁-C₆ alkyl substituted with one or more hydroxy, and R^{1b} is —CO₂R¹³; R^{1a} is C₁-C₆ alkyl substituted with one or more hydroxy, and R^{1b} is —NR¹³CONR¹¹R¹²; R^{1a} is C₁-C₆ alkyl substituted with one or more hydroxy, and R^{1b} is —CR¹¹R¹²CN; R^{1a} is C₁-C₆ alkyl substituted with one or more hydroxy, and R^{1b} is —NR¹¹SO₂R¹³; R^{1a} is C₁-C₆ alkyl substituted with one or more hydroxy, and R^{1b} is —NR¹¹CONR¹¹R¹²; R^{1a} is C₁-C₆ alkyl substituted with one or more hydroxy, and R^{1b} is —CN; R^{1a} is C₁-C₆ alkyl substituted with one or more hydroxy, and R^{1b} is —NR¹¹COR¹²; R^{1a} is C₁-C₆ alkyl substituted with one or more hydroxy, and R^{1b} is —CR¹¹R¹²NR¹¹R¹²; R^{1a} is C₁-C₆ alkyl substituted with one or more —OSi(R¹³)₃, and R^{1b} is C₁-C₆ alkyl substituted with one or more hydroxy; R^{1a} is C₁-C₆ alkyl substituted with one or more —OSi(R¹³)₃, and R^{1b} is —SO₂NR¹¹R¹²; R^{1a} is C₁-C₆ alkyl substituted with one or more —OSi(R¹³)₃, and R^{1b} is —SO₂R¹³; R^{1a} is C₁-C₆ alkyl substituted with one or more —OSi(R¹³)₃, and R^{1b} is —CONR¹¹R¹²; R^{1a} is C₁-C₆ alkyl substituted with one or more —OSi(R¹³)₃, and R^{1b} is —OR¹¹; R^{1a} is C₁-C₆ alkyl substituted with one or more —OSi(R¹³)₃, and R^{1b} is —COR¹³; R^{1a} is C₁-C₆ alkyl substituted with one or more —OSi(R¹³)₃, and R^{1b} is —CO₂R¹³; R^{1a} is C₁-C₆ alkyl substituted with one or more —OSi(R¹³)₃, and R^{1b} is —NR¹³CONR¹¹R¹²; R^{1a} is C₁-C₆ alkyl substituted with one or more —OSi(R¹³)₃, and R^{1b} is —CR¹¹R¹²CN; R^{1a} is C₁-C₆ alkyl substituted with one or more —OSi(R¹³)₃, and R^{1b} is —NR¹¹SO₂R¹³; R^{1a} is C₁-C₆ alkyl substituted with one or more —OSi(R¹³)₃, and R^{1b} is —NR¹¹CONR¹¹R¹²; R^{1a} is C₁-C₆ alkyl substituted with one or more —OSi(R¹³)₃, and R^{1b} is —CN; R^{1a} is C₁-C₆ alkyl substituted with one or more —OSi(R¹³)₃, and R^{1b} is —NR¹¹COR¹²; R^{1a} is C₁-C₆ alkyl substituted with one or more —OSi(R¹³)₃, and R^{1b} is —CR¹¹R¹²NR¹¹R¹²; R^{1a} is —SO₂NR¹¹R¹², and R^{1b} is C₁-C₆ alkyl substituted with one or more hydroxy; R^{1a} is —SO₂NR¹¹R¹², and R^{1b} is —SO₂NR¹¹R¹²; R^{1a} is —SO₂NR¹¹R¹², and R^{1b} is —SO₂R¹³; R^{1a} is —SO₂NR¹¹R¹², and R^{1b} is —CONR¹¹R¹²; R^{1a} is —SO₂NR¹¹R¹², and R^{1b} is —OR¹¹; R^{1a} is —SO₂NR¹¹R¹², and R^{1b} is —COR¹³; R^{1a} is —SO₂NR¹¹R¹², and R^{1b} is —CO₂R¹³; R^{1a} is —SO₂NR¹¹R¹², and R^{1b} is —NR¹³CONR¹¹R¹²; R^{1a} is —SO₂NR¹¹R¹², and R^{1b} is —CR¹¹R¹²CN; R^{1a} is —SO₂NR¹¹R¹², and R^{1b} is —NR¹¹SO₂R¹³; R^{1a} is —SO₂NR¹¹R¹², and R^{1b} is —CR¹¹R¹²NR¹¹R¹²; R^{1a} is —SO₂NR¹¹R¹², and R^{1b} is —CN; R^{1a} is —SO₂NR¹¹R¹², and R^{1b} is —NR¹¹CONR¹¹R¹²; R^{1a} is —SO₂NR¹¹R¹², and R^{1b} is —NR¹¹COR¹². R^{1a} is —CR¹¹R¹²NR¹¹R¹², and R^{1b} is C₁-C₆ alkyl substituted with one or more hydroxy; R^{1a} is —CR¹¹R¹²NR¹¹R¹², and R^{1b} is —SO₂NR¹¹R¹²; R^{1a} is —CR¹¹R¹²NR¹¹R¹², and R^{1b} is —SO₂R¹³; R^{1a} is —CR¹¹R¹²NR¹¹R¹², and R^{1b} is —CONR¹¹R¹²; R^{1a} is —CR¹¹R¹²NR¹¹R¹², and R^{1b} is —OR¹¹; R^{1a} is —CR¹¹R¹²NR¹¹R¹², and R^{1b} is —COR¹³; R^{1a} is —CR¹¹R¹²NR¹¹R¹², and R^{1b} is —CO₂R¹³; R^{1a} is —CR¹¹R¹²NR¹¹R¹², and R^{1b} is —NR¹³CONR¹¹R¹²; R^{1a} is —CR¹¹R¹²NR¹¹R¹², and R^{1b} is —CR¹¹R¹²CN; R^{1a} is —CR¹¹R¹²NR¹¹R¹², and R^{1b} is —NR¹¹SO₂R¹³; R^{1a} is —CR¹¹R¹²NR¹¹R¹², and R^{1b} is —CR¹¹R¹²NR¹¹R¹²; R^{1a} is —CR¹¹R¹²NR¹¹R¹², and R^{1b} is —CN; R^{1a} is —CR¹¹R¹²NR¹¹R¹², and R^{1b} is —NR¹¹CONR¹¹R¹²; and R^{1a} is —CR¹¹R¹²NR¹¹R¹², and R^{1b} is —NR¹¹COR¹².

In some embodiments of the compound of formula AA,

the substituted ring A is

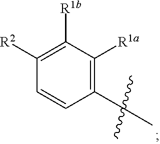

R^{1a} and R^{1b} are one of the following combinations:

In some embodiments, R^{1a} is 2-hydroxy-2-propyl, and R^{1b} is —OMe; In some embodiments, R^{1a} is 2-hydroxy-2-propyl, and R^{1b} is —OH; In some embodiments, R^{1a} is 2-hydroxy-2-propyl, and R^{1b} is —CO₂Me; In some embodiments, R^{1a} is 2-hydroxy-2-propyl, and R^{1b} is hydroxymethyl; In some embodiments, R^{1a} is 2-hydroxy-2-propyl, and R^{1b} is hydroxy ethyl; In some embodiments, R^{1a} is 2-hydroxy-2-propyl, and R^{1b} is 2-hydroxy-2-propyl; In some embodiments, R^{1a} is 2-hydroxy-2-propyl, and R^{1b} is —SO₂NHCH₂CH₂OH; In some embodiments, R^{1a} is 2-hydroxy-2-propyl, and R^{1b} is —SO₂Me; In some embodiments, R^{1a} is 2-hydroxy-2-propyl, and R^{1b} is CONHMe; In some embodiments, R^{1a} is 2-hydroxy-2-propyl, and R^{1b} is cyanomethyl; In some embodiments, R^{1a} is 2-hydroxy-2-propyl, and R^{1b} is —CN; In some embodiments, R^{1a} is 2-hydroxy-2-propyl, and R^{1b} is dimethylaminomethyl; In some embodiments, R^{1a} is dimethylaminomethyl, and R^{1b} is —OMe; In some embodiments, R^{1a} is dimethylaminomethyl, and R^{1b} is —OH; In some embodiments, R^{1a} is dimethylaminomethyl, and R^{1b} is —CO₂Me; In some embodiments, R^{1a} is dimethylaminomethyl, and R^{1b} is hydroxymethyl; In some embodiments, R^{1a} is dimethylaminomethyl, and R^{1b} is hydroxy ethyl; In some embodiments, R^{1a} is dimethylaminomethyl, and R^{1b} is 2-hydroxy-2-propyl; In some embodiments, R^{1a} is dimethylaminomethyl, and R^{1b} is —SO₂NHCH₂CH₂OH; In some embodiments, R^{1a} is dimethylaminomethyl, and R^{1b} is —SO₂Me; In some embodiments, R^{1a} is dimethylaminomethyl, and R^{1b} is CONHMe; In some embodiments, R^{1a} is dimethylaminomethyl, and R^{1b} is cyanomethyl; In some embodiments, R^{1a} is dimethylaminomethyl, and R^{1b} is dimethylaminomethyl; In some embodiments, R^{1a} is dimethylaminomethyl, and R^{1b} is CN In some embodiments, R^{1a} is —SO₂NHMe, and R^{1b} is —OMe; In some embodiments, R^{1a} is —SO₂NHMe, and R^{1b} is —OH; In some embodiments, R^{1a} is —SO₂NHMe, and R^{1b} is —CO₂Me; In some embodiments, R^{1a} is —SO₂NHMe, and R^{1b} is hydroxymethyl; In some embodiments, R^{1a} is —SO₂NHMe, and R^{1b} is hydroxyethyl; In some embodiments, R^{1a} is —SO₂NHMe, and R^{1b} is 2-hydroxy-2-propyl; In some embodiments, R^{1a} is —SO₂NHMe, and R^{1b} is —SO₂NHCH₂CH₂OH; In some embodiments, R^{1a} is —SO₂NHMe, and R^{1b} is —SO₂Me; In some embodiments, R^{1a} is —SO₂NHMe, and R^{1b} is CONHMe; In some embodiments, R^{1a} is —SO₂NHMe, and R^{1b} is dimethylaminomethyl; In some embodiments, R^{1a} is —SO₂NHMe, and R^{1b} is —CN; In some embodiments, R^{1a} is —SO₂NHMe, and R^{1b} is cyanomethyl; and In some embodiments, R^{1a} is C₁-C₄ alkyl substituted with one —OSi(Me)₂tBu, and R^{1b} is —CO₂Me.

In some embodiments of the compound of formula AA,

the substituted ring A is

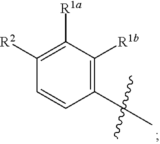

R^{1a} and R^{1b} are one of the following combinations:
R^{1a} is C₁-C₆ alkyl substituted with one or more hydroxy, and R^{1b} is C₁-C₆ alkyl substituted with one or more hydroxy; R^{1a} is C₁-C₆ alkyl substituted with one or more hydroxy, and R^{1b} is —SO₂NR¹¹R¹²; R^{1a} is C₁-C₆ alkyl substituted with one or more hydroxy, and R^{1b} is —SO₂R¹³; R^{1a} is C₁-C₆ alkyl substituted with one or more hydroxy, and R^{1b} is —CONR¹¹R¹²; R^{1a} is C₁-C₆ alkyl substituted with one or more hydroxy, and R^{1b} is —OR¹¹; R^{1a} is C₁-C₆ alkyl substituted with one or more hydroxy, and R^{1b} is —COR¹³; R^{1a} is C₁-C₆ alkyl substituted with one or more hydroxy, and R^{1b} is —CO₂R¹³; R^{1a} is C₁-C₆ alkyl substituted with one or more hydroxy, and R^{1b} is —NR¹³CONR¹¹R¹²; R^{1a} is C₁-C₆ alkyl substituted with one or more hydroxy, and R^{1b} is —CR¹¹R¹²CN; R^{1a} is C₁-C₆ alkyl substituted with one or more hydroxy, and R^{1b} is —NR¹¹SO₂R¹³; R^{1a} is C₁-C₆ alkyl substituted with one or more hydroxy, and R^{1b} is —NR¹¹CONR¹¹R¹²; R^{1a} is C₁-C₆ alkyl substituted with one or more hydroxy, and R^{1b} is —CN; R^{1a} is C₁-C₆ alkyl substituted with one or more hydroxy, and R^{1b} is —NR¹¹COR¹²; R^{1a} is C₁-C₆ alkyl substituted with one or more hydroxy, and R^{1b} is —CR¹¹R¹²NR¹¹R¹²; R^{1a} is C₁-C₆ alkyl substituted with one or more —OSi(R¹³)₃, and R^{1b} is C₁-C₆ alkyl substituted with one or more hydroxy; R^{1a} is C₁-C₆ alkyl substituted with one or more —OSi(R¹³)₃, and R^{1b} is —SO₂NR¹¹R¹²; R^{1a} is C₁-C₆ alkyl substituted with one or more —OSi(R¹³)₃, and R^{1b} is —SO₂R¹³; R^{1a} is C₁-C₆ alkyl substituted with one or more —OSi(R¹³)₃, and R^{1b} is —CONR¹¹R¹²; R^{1a} is C₁-C₆ alkyl substituted with one or more —OSi(R¹³)₃, and R^{1b} is —OR¹¹; R^{1a} is C₁-C₆ alkyl substituted with one or more —OSi(R¹³)₃, and R^{1b} is —COR¹³; R^{1a} is C₁-C₆ alkyl substituted with one or more —OSi(R¹³)₃, and R^{1b} is —CO₂R¹³; R^{1a} is C₁-C₆ alkyl substituted with one or more —OSi(R¹³)₃, and R^{1b} is —NR¹³CONR¹¹R¹²; R^{1a} is C₁-C₆ alkyl substituted with one or more —OSi(R¹³)₃, and R^{1b} is —CR¹¹R¹²CN; R^{1a} is C₁-C₆ alkyl substituted with one or more —OSi(R¹³)₃, and R^{1b} is —NR¹¹SO₂R¹³; R^{1a} is C₁-C₆ alkyl substituted with one or more —OSi(R¹³)₃, and R^{1b} is —NR¹¹CONR¹¹R¹²; R^{1a} is C₁-C₆ alkyl substituted with one or more —OSi(R¹³)₃, and R^{1b} is —CN; R^{1a} is C₁-C₆ alkyl substituted with one or more —OSi(R¹³)₃, and R^{1b} is —NR¹¹COR¹²; R^{1a} is C₁-C₆ alkyl substituted with one or more —OSi(R¹³)₃, and R^{1b} is —CR¹¹R¹²NR¹¹R¹²; R^{1a} is —SO₂NR¹¹R¹², and R^{1b} is C₁-C₆ alkyl substituted with one or more hydroxy; R^{1a} is —SO₂NR¹¹R¹², and R^{1b} is —SO₂NR¹¹R¹²; R^{1a} is —SO₂NR¹¹R¹², and R^{1b} is —SO₂R¹³; R^{1a} is —SO₂NR¹¹R¹², and R^{1b} is —CONR¹¹R¹²; R^{1a} is —SO₂NR¹¹R¹², and R^{1b} is —OR¹¹; R^{1a} is —SO₂NR¹¹R¹², and R^{1b} is —COR¹³; R^{1a} is —SO₂NR¹¹R¹², and R^{1b} is —CO₂R¹³; R^{1a} is —SO₂NR¹¹R¹², and R^{1b} is —NR¹³CONR¹¹R¹²; R^{1a} is —SO₂NR¹¹R¹², and R^{1b} is —CR¹¹R¹²CN; R^{1a} is —SO₂NR¹¹R¹², and R^{1b} is —NR¹¹SO₂R¹³; R^{1a} is —SO₂NR¹¹R¹², and R^{1b} is —CR¹¹R¹²NR¹¹R¹²; R^{1a} is —SO₂NR¹¹R¹², and R^{1b} is —CN; R^{1a} is —SO₂NR¹¹R¹², and R^{1b} is —NR¹¹CONR¹¹R¹²; R^{1a} is —SO₂NR¹¹R¹², and R^{1b} is —NR¹¹COR¹². R^{1a} is —CR¹¹R¹²NR¹¹R¹², and R^{1b} is C₁-C₆ alkyl substituted with one or more hydroxy; R^{1a} is —CR¹¹R¹²NR¹¹R¹², and R^{1b} is —SO₂NR¹¹R¹²; R^{1a} is —CR¹¹R¹²NR¹¹R¹², and R^{1b} is —SO₂R¹³; R^{1a} is —CR¹¹R¹²NR¹¹R¹², and R^{1b} is —CONR¹¹R¹²; R^{1a} is —CR¹¹R¹²NR¹¹R¹², and R^{1b} is —OR¹¹; R^{1a} is —CR¹¹R¹²NR¹¹R¹², and R^{1b} is —COR¹³; R^{1a} is —CR¹¹R¹²NR¹¹R¹², and R^{1b} is —CO₂R¹³; R^{1a} is —CR¹¹R¹²NR¹¹R¹², and R^{1b} is —NR¹³CONR¹¹R¹²; R^{1a} is —CR¹¹R¹²NR¹¹R¹², and R^{1b} is —CR¹¹R¹²CN; R^{1a} is —CR¹¹R¹²NR¹¹R¹², and R^{1b} is —NR¹¹SO₂R¹³; R^{1a} is —CR¹¹R¹²NR¹¹R¹², and R^{1b} is —CR¹¹R¹²NR¹¹R¹²; R^{1a} is —CR¹¹R¹²NR¹¹R¹², and R^{1b} is —CN; R^{1a} is —CR¹¹R¹²NR¹¹R¹², and R^{1b} is —NR¹¹CONR¹¹R¹²; and R^{1a} is —CR¹¹R¹²NR¹¹R¹², and R^{1b} is —NR¹¹COR¹².

In some embodiments of the compound of formula AA,

the substituted ring A is

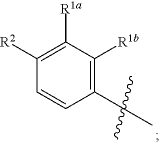

R^{1a} and R^{1b} are one of the following combinations:

In some embodiments, R^{1a} is 2-hydroxy-2-propyl, and R^{1b} is —OMe; In some embodiments, R^{1a} is 2-hydroxy-2-propyl, and R^{1b} is —OH; In some embodiments, R^{1a} is 2-hydroxy-2-propyl, and R^{1b} is —CO₂Me; In some embodiments, R^{1a} is 2-hydroxy-2-propyl, and R^{1b} is hydroxymethyl; In some embodiments, R^{1a} is 2-hydroxy-2-propyl, and R^{1b} is hydroxy ethyl; In some embodiments, R^{1a} is 2-hydroxy-2-propyl, and R^{1b} is 2-hydroxy-2-propyl; In some embodiments, R^{1a} is 2-hydroxy-2-propyl, and R^{1b} is —SO₂NHCH₂CH₂OH; In some embodiments, R^{1a} is 2-hydroxy-2-propyl, and R^{1b} is —SO₂Me; In some embodiments, R^{1a} is 2-hydroxy-2-propyl, and R^{1b} is CONHMe; In some embodiments, R^{1a} is 2-hydroxy-2-propyl, and R^{1b} is cyanomethyl; In some embodiments, R^{1a} is 2-hydroxy-2-propyl, and R^{1b} is —CN; In some embodiments, R^{1a} is 2-hydroxy-2-propyl, and R^{1b} is dimethylaminomethyl; In some embodiments, R^{1a} is dimethylaminomethyl, and R^{1b} is —OMe; In some embodiments, R^{1a} is dimethylaminomethyl, and R^{1b} is —OH; In some embodiments, R^{1a} is dimethylaminomethyl, and R^{1b} is —CO₂Me; In some embodiments, R^{1a} is dimethylaminomethyl, and R^{1b} is hydroxymethyl; In some embodiments, R^{1a} is dimethylaminomethyl, and R^{1b} is hydroxy ethyl; In some embodiments, R^{1a} is dimethylaminomethyl, and R^{1b} is 2-hydroxy-2-propyl; In some embodiments, R^{1a} is dimethylaminomethyl, and R^{1b} is —SO₂NHCH₂CH₂OH; In some embodiments, R^{1a} is dimethylaminomethyl, and R^{1b} is —SO₂Me; In some embodiments, R^{1a} is dimethylaminomethyl, and R^{1b} is CONHMe; In some embodiments, R^{1a} is dimethylaminomethyl, and R^{1b} is cyanomethyl; In some embodiments, R^{1a} is dimethylaminomethyl, and R^{1b} is dimethylaminomethyl; In some embodiments, R^{1a} is dimethylaminomethyl, and R^{1b} is CN; In some embodiments, R^{1a} is —SO₂NHMe, and R^{1b} is —OMe; In some embodiments, R^{1a} is —SO₂NHMe, and R^{1b} is —OH; In some embodiments, R^{1a} is —SO₂NHMe, and R^{1b} is —CO₂Me; In some embodiments, R^{1a} is —SO₂NHMe, and R^{1b} is hydroxymethyl; In some embodiments, R^{1a} is —SO₂NHMe, and R^{1b} is hydroxyethyl; In some embodiments, R^{1a} is —SO₂NHMe, and R^{1b} is 2-hydroxy-2-propyl; In some embodiments, R^{1a} is —SO₂NHMe, and R^{1b} is —SO₂NHCH₂CH₂OH; In some embodiments, R^{1a} is —SO₂NHMe, and R^{1b} is —SO₂Me; In some embodiments, R^{1a} is —SO₂NHMe, and R^{1b} is CONHMe; In some embodiments, R^{1a} is —SO₂NHMe, and R^{1b} is dimethylaminomethyl; In some embodiments, R^{1a} is —SO₂NHMe, and R^{1b} is —CN; In some embodiments, R^{1a} is —SO₂NHMe, and R^{1b} is cyanomethyl; and In some embodiments, R^{1a} is C₁-C₄ alkyl substituted with one —OSi(Me)₂tBu, and R^{1b} is —CO₂Me.

In some embodiments of the compound of formula AA,

the substituted ring A is

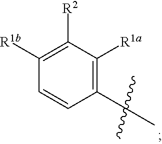

R^{1a} and R^{1b} are one of the following combinations:
R^{1a} is C₁-C₆ alkyl substituted with one or more hydroxy, and R^{1b} is C₁-C₆ alkyl substituted with one or more hydroxy; R^{1a} is C₁-C₆ alkyl substituted with one or more hydroxy, and R^{1b} is —SO₂NR¹¹R¹²; R^{1a} is C₁-C₆ alkyl substituted with one or more hydroxy, and R^{1b} is —SO₂R¹³; R^{1a} is C₁-C₆ alkyl substituted with one or more hydroxy, and R^{1b} is —CONR¹¹R¹²; R^{1a} is C₁-C₆ alkyl substituted with one or more hydroxy, and R^{1b} is —OR¹¹; R^{1a} is C₁-C₆ alkyl substituted with one or more hydroxy, and R^{1b} is —COR¹³; R^{1a} is C₁-C₆ alkyl substituted with one or more hydroxy, and R^{1b} is —CO₂R¹³; R^{1a} is C₁-C₆ alkyl substituted with one or more hydroxy, and R^{1b} is —NR¹³CONR¹¹R¹²; R^{1a} is C₁-C₆ alkyl substituted with one or more hydroxy, and R^{1b} is —CR¹¹R¹²CN; R^{1a} is C₁-C₆ alkyl substituted with one or more hydroxy, and R^{1b} is —NR¹¹SO₂R¹³; R^{1a} is C₁-C₆ alkyl substituted with one or more hydroxy, and R^{1b} is —NR¹¹CONR¹¹R¹²; R^{1a} is C₁-C₆ alkyl substituted with one or more hydroxy, and R^{1b} is —CN; R^{1a} is C₁-C₆ alkyl substituted with one or more hydroxy, and R^{1b} is —NR¹¹COR¹²; R^{1a} is C₁-C₆ alkyl substituted with one or more hydroxy, and R^{1b} is —CR¹¹R¹²NR¹¹R¹²; R^{1a} is C₁-C₆ alkyl substituted with one or more —OSi(R¹³)₃, and R^{1b} is C₁-C₆ alkyl substituted with one or more hydroxy; R^{1a} is C₁-C₆ alkyl substituted with one or more —OSi(R¹³)₃, and R^{1b} is —SO₂NR¹¹R¹²; R^{1a} is C₁-C₆ alkyl substituted with one or more —OSi(R¹³)₃, and R^{1b} is —SO₂R¹³; R^{1a} is C₁-C₆ alkyl substituted with one or more —OSi(R¹³)₃, and R^{1b} is —CONR¹¹R¹²; R^{1a} is C₁-C₆ alkyl substituted with one or more —OSi(R¹³)₃, and R^{1b} is —OR¹¹; R^{1a} is C₁-C₆ alkyl substituted with one or more —OSi(R¹³)₃, and R^{1b} is —COR¹³; R^{1a} is C₁-C₆ alkyl substituted with one or more —OSi(R¹³)₃, and R^{1b} is —CO₂R¹³; R^{1a} is C₁-C₆ alkyl substituted with one or more —OSi(R¹³)₃, and R^{1b} is —NR¹³CONR¹¹R¹²; R^{1a} is C₁-C₆ alkyl substituted with one or more —OSi(R¹³)₃, and R^{1b} is —CR¹¹R¹²CN; R^{1a} is C₁-C₆ alkyl substituted with one or more —OSi(R¹³)₃, and R^{1b} is —NR¹¹SO₂R¹³; R^{1a} is C₁-C₆ alkyl substituted with one or more —OSi(R¹³)₃, and R^{1b} is —NR¹¹CONR¹¹R¹²; R^{1a} is C₁-C₆ alkyl substituted with one or more —OSi(R¹³)₃, and R^{1b} is —CN; R^{1a} is C₁-C₆ alkyl substituted with one or more —OSi(R¹³)₃, and R^{1b} is —NR¹¹COR¹²; R^{1a} is C₁-C₆ alkyl substituted with one or more —OSi(R¹³)₃, and R^{1b} is —CR¹¹R¹²NR¹¹R¹²; R^{1a} is —SO₂NR¹¹R¹², and R^{1b} is C₁-C₆ alkyl substituted with one or more hydroxy; R^{1a} is —SO₂NR¹¹R¹², and R^{1b} is —SO₂NR¹¹R¹²; R^{1a} is —SO₂NR¹¹R¹², and R^{1b} is —SO₂R¹³; R^{1a} is —SO₂NR¹¹R¹², and R^{1b} is —CONR¹¹R¹²; R^{1a} is —SO₂NR¹¹R¹², and R^{1b} is —OR¹¹; R^{1a} is —SO₂NR¹¹R¹², and R^{1b} is —COR¹³; R^{1a} is —SO₂NR¹¹R¹², and R^{1b} is —CO₂R¹³; R^{1a} is —SO₂NR¹¹R¹², and R^{1b} is —NR¹³CONR¹¹R¹²; R^{1a} is —SO₂NR¹¹R¹², and R^{1b} is —CR¹¹R¹²CN; R^{1a} is —SO₂NR¹¹R¹², and R^{1b} is —NR¹¹SO₂R¹³; R^{1a} is —SO₂NR¹¹R¹², and R^{1b} is —CR¹¹R¹²NR¹¹R¹²; R^{1a} is —SO₂NR¹¹R¹², and R^{1b} is —CN R^{1a} is —SO₂NR¹¹R¹², and R^{1b} is —NR¹¹CONR¹¹R¹²; R^{1a} is —SO₂NR¹¹R¹², and R^{1b} is —NR¹¹COR¹². R^{1a} is —CR¹¹R¹²NR¹¹R¹², and R^{1b} is C₁-C₆ alkyl substituted with one or more hydroxy; R^{1a} is —CR¹¹R¹²NR¹¹R¹², and R^{1b} is —SO₂NR¹¹R¹²; R^{1a} is —CR¹¹R¹²NR¹¹R¹², and R^{1b} is —SO₂R¹³; R^{1a} is —CR¹¹R¹²NR¹¹R¹², and R^{1b} is —CONR¹¹R¹²; R^{1a} is —CR¹¹R¹²NR¹¹R¹², and R^{1b} is —OR¹¹; R^{1a} is —CR¹¹R¹²NR¹¹R¹², and R^{1b} is —COR¹³; R^{1a} is —CR¹¹R¹²NR¹¹R¹², and R^{1b} is —CO₂R¹³; R^{1a} is —CR¹¹R¹²NR¹¹R¹², and R^{1b} is —NR¹³CONR¹¹R¹²; R^{1a} is —CR¹¹R¹²NR¹¹R¹², and R^{1b} is —CR¹¹R¹²CN; R^{1a} is —CR¹¹R¹²NR¹¹R¹², and R^{1b} is —NR¹¹SO₂R¹³; R^{1a} is —CR¹¹R¹²NR¹¹R¹², and R^{1b} is —CR¹¹R¹²NR¹¹R¹²; R^{1a} is —CR¹¹R¹²NR¹¹R¹², and R^{1b} is —CN; R^{1a} is —CR¹¹R¹²NR¹¹R¹², and R^{1b} is —NR¹¹CONR¹¹R¹²; and R^{1a} is —CR¹¹R¹²NR¹¹R¹², and R^{1b} is —NR¹¹COR¹².

In some embodiments of the compound of formula AA,

the substituted ring A is

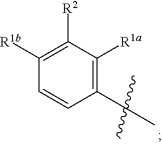

R^{1a} and R^{1b} are one of the following combinations:

In some embodiments, R^{1a} is 2-hydroxy-2-propyl, and R^{1b} is —OMe; In some embodiments, R^{1a} is 2-hydroxy-2-propyl, and R^{1b} is —OH; In some embodiments, R^{1a} is 2-hydroxy-2-propyl, and R^{1b} is —CO₂Me; In some embodiments, R^{1a} is 2-hydroxy-2-propyl, and R^{1b} is hydroxymethyl; In some embodiments, R^{1a} is 2-hydroxy-2-propyl, and R^{1b} is hydroxy ethyl; In some embodiments, R^{1a} is 2-hydroxy-2-propyl, and R^{1b} is 2-hydroxy-2-propyl; In some embodiments, R^{1a} is 2-hydroxy-2-propyl, and R^{1b} is —SO₂NHCH₂CH₂OH; In some embodiments, R^{1a} is 2-hydroxy-2-propyl, and R^{1b} is —SO₂Me; In some embodiments, R^{1a} is 2-hydroxy-2-propyl, and R^{1b} is CONHMe; In some embodiments, R^{1a} is 2-hydroxy-2-propyl, and R^{1b} is cyanomethyl; In some embodiments, R^{1a} is 2-hydroxy-2-propyl, and R^{1b} is —CN; In some embodiments, R^{1a} is 2-hydroxy-2-propyl, and R^{1b} is dimethylaminomethyl; In some embodiments, R^{1a} is dimethylaminomethyl, and R^{1b} is —OMe; In some embodiments, R^{1a} is dimethylaminomethyl, and R^{1b} is —OH; In some embodiments, R^{1a} is dimethylaminomethyl, and R^{1b} is —CO₂Me; In some embodiments, R^{1a} is dimethylaminomethyl, and R^{1b} is hydroxymethyl; In some embodiments, R^{1a} is dimethylaminomethyl, and R^{1b} is hydroxy ethyl; In some embodiments, R^{1a} is dimethylaminomethyl, and R^{1b} is 2-hydroxy-2-propyl; In some embodiments, R^{1a} is dimethylaminomethyl, and R^{1b} is —SO₂NHCH₂CH₂OH; In some embodiments, R^{1a} is dimethylaminomethyl, and R^{1b} is —SO₂Me; In some embodiments, R^{1a} is dimethylaminomethyl, and R^{1b} is CONHMe; In some embodiments, R^{1a} is dimethylaminomethyl, and R^{1b} is cyanomethyl; In some embodiments, R^{1a} is dimethylaminomethyl, and R^{1b} is dimethylaminomethyl; In some embodiments, R^{1a} is dimethylaminomethyl, and R^{1b} is CN; In some embodiments, R^{1a} is —SO₂NHMe, and R^{1b} is —OMe; In some embodiments, R^{1a} is —SO₂NHMe, and R^{1b} is —OH; In some embodiments, R^{1a} is —SO₂NHMe, and R^{1b} is —CO₂Me; In some embodiments, R^{1a} is —SO₂NHMe, and R^{1b} is hydroxymethyl; In some embodiments, R^{1a} is —SO₂NHMe, and R^{1b} is hydroxyethyl; In some embodiments, R^{1a} is —SO₂NHMe, and R^{1b} is 2-hydroxy-2-propyl; In some embodiments, R^{1a} is —SO₂NHMe, and R^{1b} is —SO₂NHCH₂CH₂OH; In some embodiments, R^{1a} is —SO₂NHMe, and R^{1b} is —SO₂Me; In some embodiments, R^{1a} is —SO₂NHMe, and R^{1b} is CONHMe; In some embodiments, R^{1a} is —SO₂NHMe, and R^{1b} is dimethylaminomethyl; In some embodiments, R^{1a} is —SO₂NHMe, and R^{1b} is —CN; In some embodiments, R^{1a} is —SO₂NHMe, and R^{1b} is cyanomethyl; and In some embodiments, R^{1a} is C₁-C₄ alkyl substituted with one —OSi(Me)₂tBu, and R^{1b} is —CO₂Me.

In some embodiments of the compound of formula AA,

the substituted ring A is

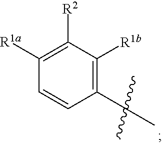

R^{1a} and R^{1b} are one of the following combinations:
R^{1a} is C₁-C₆ alkyl substituted with one or more hydroxy, and R^{1b} is C₁-C₆ alkyl substituted with one or more hydroxy; R^{1a} is C₁-C₆ alkyl substituted with one or more hydroxy, and R^{1b} is —SO₂NR¹¹R¹²; R^{1a} is C₁-C₆ alkyl substituted with one or more hydroxy, and R^{1b} is —SO₂R¹³; R^{1a} is C₁-C₆ alkyl substituted with one or more hydroxy, and R^{1b} is —CONR¹¹R¹²; R^{1a} is C₁-C₆ alkyl substituted with one or more hydroxy, and R^{1b} is —OR¹¹; R^{1a} is C₁-C₆ alkyl substituted with one or more hydroxy, and R^{1b} is —COR¹³; R^{1a} is C₁-C₆ alkyl substituted with one or more hydroxy, and R^{1b} is —CO₂R¹³; R^{1a} is C₁-C₆ alkyl substituted with one or more hydroxy, and R^{1b} is —NR¹³CONR¹¹R¹²; R^{1a} is C₁-C₆ alkyl substituted with one or more hydroxy, and R^{1b} is —CR¹¹R¹²CN; R^{1a} is C₁-C₆ alkyl substituted with one or more hydroxy, and R^{1b} is —NR¹¹SO₂R¹³; R^{1a} is C₁-C₆ alkyl substituted with one or more hydroxy, and R^{1b} is —NR¹¹CONR¹¹R¹²; R^{1a} is C₁-C₆ alkyl substituted with one or more hydroxy, and R^{1b} is —CN; R^{1a} is C₁-C₆ alkyl substituted with one or more hydroxy, and R^{1b} is —NR¹¹COR¹²; R^{1a} is C₁-C₆ alkyl substituted with one or more hydroxy, and R^{1b} is —CR¹¹R¹²NR¹¹R¹²; R^{1a} is C₁-C₆ alkyl substituted with one or more —OSi(R¹³)₃, and R^{1b} is C₁-C₆ alkyl substituted with one or more hydroxy; R^{1a} is C₁-C₆ alkyl substituted with one or more —OSi(R¹³)₃, and R^{1b} is —SO₂NR¹¹R¹²; R^{1a} is C₁-C₆ alkyl substituted with one or more —OSi(R¹³)₃, and R^{1b} is —SO₂R¹³; R^{1a} is C₁-C₆ alkyl substituted with one or more —OSi(R¹³)₃, and R^{1b} is —CONR¹¹R¹²; R^{1a} is C₁-C₆ alkyl substituted with one or more —OSi(R¹³)₃, and R^{1b} is —OR¹¹; R^{1a} is C₁-C₆ alkyl substituted with one or more —OSi(R¹³)₃, and R^{1b} is —COR¹³; R^{1a} is C₁-C₆ alkyl substituted with one or more —OSi(R¹³)₃, and R^{1b} is —CO₂R¹³; R^{1a} is C₁-C₆ alkyl substituted with one or more —OSi(R¹³)₃, and R^{1b} is —NR¹³CONR¹¹R¹²; R^{1a} is C₁-C₆ alkyl substituted with one or more —OSi(R¹³)₃, and R^{1b} is —CR¹¹R¹²CN; R^{1a} is C₁-C₆ alkyl substituted with one or more —OSi(R¹³)₃, and R^{1b} is —NR¹¹SO₂R¹³; R^{1a} is C₁-C₆ alkyl substituted with one or more —OSi(R¹³)₃, and R^{1b} is —NR¹¹CONR¹¹R¹²; R^{1a} is C₁-C₆ alkyl substituted with one or more —OSi(R¹³)₃, and R^{1b} is —CN; R^{1a} is C₁-C₆ alkyl substituted with one or more —OSi(R¹³)₃, and R^{1b} is —NR¹¹COR¹²; R^{1a} is C₁-C₆ alkyl substituted with one or more —OSi(R¹³)₃, and R^{1b} is —CR¹¹R¹²NR¹¹R¹²; R^{1a} is —SO₂NR¹¹R¹², and R^{1b} is C₁-C₆ alkyl substituted with one or more hydroxy; R^{1a} is —SO₂NR¹¹R¹², and R^{1b} is —SO₂NR¹¹R¹²; R^{1a} is —SO₂NR¹¹R¹², and R^{1b} is —SO₂R¹³; R^{1a} is —SO₂NR¹¹R¹², and R^{1b} is —CONR¹¹R¹²; R^{1a} is —SO₂NR¹¹R¹², and R^{1b} is —OR¹¹; R^{1a} is —SO₂NR¹¹R¹², and R^{1b} is —COR¹³; R^{1a} is —SO₂NR¹¹R¹², and R^{1b} is —CO₂R¹³; R^{1a} is —SO₂NR¹¹R¹², and R^{1b} is —NR¹³CONR¹¹R¹²; R^{1a} is —SO₂NR¹¹R¹², and R^{1b} is —CR¹¹R¹²CN; R^{1a} is —SO₂NR¹¹R¹², and R^{1b} is —NR¹¹SO₂R¹³; R^{1a} is —SO₂NR¹¹R¹², and R^{1b} is —CR¹¹R¹²NR¹¹R¹²; R^{1a} is —SO₂NR¹¹R¹², and R^{1b} is —CN; R^{1a} is —SO₂NR¹¹R¹², and R^{1b} is —NR¹¹CONR¹¹R¹²; and R^{1a} is —SO₂NR¹¹R¹², and R^{1b} is —NR¹¹COR¹²; R^{1a} is —CR¹¹R¹²NR¹¹R¹², and R^{1b} is C₁-C₆ alkyl substituted with one or more hydroxy; R^{1a} is —CR¹¹R¹²NR¹¹R¹², and R^{1b} is —SO₂NR¹¹R¹²; R^{1a} is —CR¹¹R¹²NR¹¹R¹², and R^{1b} is —SO₂R¹³; R^{1a} is —CR¹¹R¹²NR¹¹R¹², and R^{1b} is —CONR¹¹R¹²; R^{1a} is —CR¹¹R¹²NR¹¹R¹², and R^{1b} is —OR¹¹; R^{1a} is —CR¹¹R¹²NR¹¹R¹², and R^{1b} is —COR¹³; R^{1a} is —CR¹¹R¹²NR¹¹R¹², and R^{1b} is —CO₂R¹³; R^{1a} is —CR¹¹R¹²NR¹¹R¹², and R^{1b} is —NR¹³CONR¹¹R¹²; R^{1a} is —CR¹¹R¹²NR¹¹R¹², and R^{1b} is —CR¹¹R¹²CN; R^{1a} is —CR¹¹R¹²NR¹¹R¹², and R^{1b} is —NR¹¹SO₂R¹³; R^{1a} is —CR¹¹R¹²NR¹¹R¹², and R^{1b} is —CR¹¹R¹²NR¹¹R¹²; R^{1a} is —CR¹¹R¹²NR¹¹R¹², and R^{1b} is —CN; R^{1a} is —CR¹¹R¹²NR¹¹R¹², and R^{1b} is —NR¹¹CONR¹¹R¹²; and R^{1a} is —CR¹¹R¹²NR¹¹R¹², and R^{1b} is —NR¹¹COR¹².

In some embodiments of the compound of formula AA, the substituted ring A is

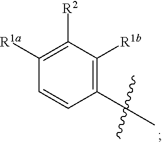

R^{1a} and R^{1b} are one of the following combinations:

In some embodiments, R^{1a} is 2-hydroxy-2-propyl, and R^{1b} is —OMe; In some embodiments, R^{1a} is 2-hydroxy-2-propyl, and R^{1b} is —OH; In some embodiments, R^{1a} is 2-hydroxy-2-propyl, and R^{1b} is —CO₂Me; In some embodiments, R^{1a} is 2-hydroxy-2-propyl, and R^{1b} is hydroxymethyl; In some embodiments, R^{1a} is 2-hydroxy-2-propyl, and R^{1b} is hydroxy ethyl; In some embodiments, R^{1a} is 2-hydroxy-2-propyl, and R^{1b} is 2-hydroxy-2-propyl; In some embodiments, R^{1a} is 2-hydroxy-2-propyl, and R^{1b} is —SO₂NHCH₂CH₂OH; In some embodiments, R^{1a} is 2-hydroxy-2-propyl, and R^{1b} is —SO₂Me; In some embodiments, R^{1a} is 2-hydroxy-2-propyl, and R^{1b} is CONHMe; In some embodiments, R^{1a} is 2-hydroxy-2-propyl, and R^{1b} is cyanomethyl; In some embodiments, R^{1a} is 2-hydroxy-2-propyl, and R^{1b} is —CN; In some embodiments, R^{1a} is 2-hydroxy-2-propyl, and R^{1b} is dimethylaminomethyl; In some embodiments, R^{1a} is dimethylaminomethyl, and R^{1b} is —OMe; In some embodiments, R^{1a} is dimethylaminomethyl, and R^{1b} is —OH; In some embodiments, R^{1a} is dimethylaminomethyl, and R^{1b} is —CO₂Me; In some embodiments, R^{1a} is dimethylaminomethyl, and R^{1b} is hydroxymethyl; In some embodiments, R^{1a} is dimethylaminomethyl, and R^{1b} is hydroxy ethyl; In some embodiments, R^{1a} is dimethylaminomethyl, and R^{1b} is 2-hydroxy-2-propyl; In some embodiments, R^{1a} is dimethylaminomethyl, and R^{1b} is —SO₂NHCH₂CH₂OH; In some embodiments, R^{1a} is dimethylaminomethyl, and R^{1b} is —SO₂Me; In some embodiments, R^{1a} is dimethylaminomethyl, and R^{1b} is CONHMe; In some embodiments, R^{1a} is dimethylaminomethyl, and R^{1b} is cyanomethyl; In some embodiments, R^{1a} is dimethylaminomethyl, and R^{1b} is dimethylaminomethyl; In some embodiments, R^{1a} is dimethylaminomethyl, and R^{1b} is CN; In some embodiments, R^{1a} is —SO₂NHMe, and R^{1b} is —OMe; In some embodiments, R^{1a} is —SO₂NHMe, and R^{1b} is —OH; In some embodiments, R^{1a} is —SO₂NHMe, and R^{1b} is —CO₂Me; In some embodiments, R^{1a} is —SO₂NHMe, and R^{1b} is hydroxymethyl; In some embodiments, R^{1a} is —SO₂NHMe, and R^{1b} is hydroxyethyl; In some embodiments, R^{1a} is —SO₂NHMe, and R^{1b} is 2-hydroxy-2-propyl; In some embodiments, R^{1a} is —SO₂NHMe, and R^{1b} is —SO₂NHCH₂CH₂OH; In some embodiments, R^{1a} is —SO₂NHMe, and R^{1b} is —SO₂Me; In some embodiments, R^{1a} is —SO₂NHMe, and R^{1b} is CONHMe; In some embodiments, R^{1a} is —SO₂NHMe, and R^{1b} is cyanomethyl; and In some embodiments, R^{1a} is —SO₂NHMe, and R^{1b} is dimethylaminomethyl; In some embodiments, R^{1a} is —SO₂NHMe, and R^{1b} is —CN; In some embodiments, R^{1a} is C₁-C₄ alkyl substituted with one —OSi(Me)₂tBu, and R^{1b} is —CO₂Me.

In some embodiments of the compound of formula AA,

the optionally substituted ring B is

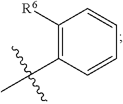

and R⁶ is selected from:

C₁-C₆ alkyl, C₁-C₆ alkyl substituted with one or more halo, C₁-C₆ alkoxy, C₁-C₆ alkoxy substituted with one or more halo, C₃-C₇ cycloalkyl, halo, and cyano.

In some embodiments of the compound of formula AA,

the optionally substituted ring B is

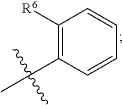

and R⁶ is selected from:

isopropyl, ethyl, methyl, trifluoromethyl, trifluoromethoxy, cyclopropyl, chloro, and fluoro.

In some embodiments of the compound of formula AA,

the optionally substituted ring B is

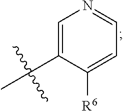

and R⁶ is selected from:

C₁-C₆ alkyl, C₁-C₆ alkyl substituted with one or more halo, C₁-C₆ alkoxy, C₁-C₆ alkoxy substituted with one or more halo, C₃-C₇ cycloalkyl, halo, and cyano.

In some embodiments of the compound of formula AA,

the optionally substituted ring B is

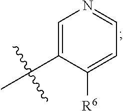

and R⁶ is selected from:

isopropyl, ethyl, methyl, trifluoromethyl, trifluoromethoxy, cyclopropyl, chloro, and fluoro.

In some embodiments of the compound of formula AA,

the optionally substituted ring B is

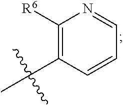

and R⁶ is selected from:

C₁-C₆ alkyl, C₁-C₆ alkyl substituted with one or more halo, C₁-C₆ alkoxy, C₁-C₆ alkoxy substituted with one or more halo, C₃-C₇ cycloalkyl, halo, and cyano.

In some embodiments of the compound of formula AA,

the optionally substituted ring B is

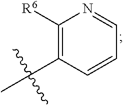

and R⁶ is selected from:

isopropyl, ethyl, methyl, trifluoromethyl, trifluoromethoxy, cyclopropyl, chloro, and fluoro.

In some embodiments of the compound of formula AA,

the optionally substituted ring B is

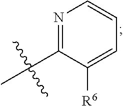

and R⁶ is selected from:

C₁-C₆ alkyl, C₁-C₆ alkyl substituted with one or more halo, C₁-C₆ alkoxy, C₁-C₆ alkoxy substituted with one or more halo, C₃-C₇ cycloalkyl, halo, and cyano.

In some embodiments of the compound of formula AA,

the optionally substituted ring B is

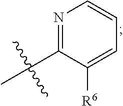

and R⁶ is selected from:

isopropyl, ethyl, methyl, trifluoromethyl, trifluoromethoxy, cyclopropyl, chloro, and fluoro.

In some embodiments of the compound of formula AA,

the optionally substituted ring B is

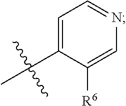

and R⁶ is selected from:

C₁-C₆ alkyl, C₁-C₆ alkyl substituted with one or more halo, C₁-C₆ alkoxy, C₁-C₆ alkoxy substituted with one or more halo, C₃-C₇ cycloalkyl, halo, and cyano.

In some embodiments of the compound of formula AA,

the optionally substituted ring B is

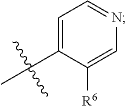

and R⁶ is selected from:

isopropyl, ethyl, methyl, trifluoromethyl, trifluoromethoxy, cyclopropyl, chloro, and fluoro.

In some embodiments, of the compound of formula AA,

the optionally substituted ring B is

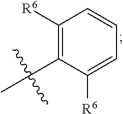

and the two R⁶ are one of the following combinations:
One R⁶ is C₁-C₆ alkyl, and the other R⁶ is C₁-C₆ alkyl optionally substituted with one or more halo; One R⁶ is C₁-C₆ alkyl and the other R⁶ is C₁-C₆ alkyl; One R⁶ is C₁-C₆ alkyl, and the other R⁶ is C₁-C₆ alkyl substituted with one or more halo; One R⁶ is C₁-C₆ alkyl, and the other R⁶ is C₃-C₇ cycloalkyl; One R⁶ is C₁-C₆ alkyl, and the other R⁶ is halo; One R⁶ is C₁-C₆ alkyl, and the other R⁶ is cyano; One R⁶ is C₃-C₇ cycloalkyl, and the other R⁶ is C₃-C₇ cycloalkyl; One R⁶ is C₃-C₇ cycloalkyl, and the other R⁶ is halo; One R⁶ is cyclopropyl and the other R⁶ is halo; One R⁶ is C₁-C₆ alkyl, and the other R⁶ is C₁-C₆ alkoxy optionally substituted with one or more halo; One R⁶ is C₁-C₆ alkyl, and the other R⁶ is C₁-C₆ alkoxy; One R⁶ is C₁-C₆ alkyl, and the other R⁶ is C₁-C₆, alkoxy substituted with one or more halo; One R⁶ is halo, and the other R⁶ is C₁-C₆ haloalkyl; One R⁶ is halo, and the other R⁶ is C₁-C₆ haloalkoxy; One R⁶ is C₁-C₆ alkoxy; and the other R⁶ is halo; One R⁶ is C₁-C₆ alkoxy; and the other R⁶ is chloro.

In some embodiments, of the compound of formula AA,

the optionally substituted ring B is

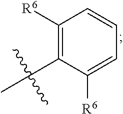

and the two R⁶ are one of the following combinations:
One R⁶ is isopropyl; and the other R⁶ is methyl; One R⁶ is isopropyl; and the other R⁶ is n-propyl; One R⁶ is isopropyl; and the other R⁶ is isopropyl; One R⁶ is isopropyl; and the other R⁶ is trifluoromethyl; One R⁶ is isopropyl; and the other R⁶ is cyclopropyl; One R⁶ is isopropyl; and the other R⁶ is chloro; One R⁶ is isopropyl; and the other R⁶ is fluoro; One R⁶ is ethyl; and the other R⁶ is fluoro; One R⁶ is isopropyl; and the other R⁶ is cyano; One R⁶ is cyclopropyl; and the other R⁶ is cyclopropyl; One R⁶ is cyclopropyl; and the other R⁶ is chloro; One R⁶ is cyclopropyl; and the other R⁶ is fluoro; One R⁶ is isopropyl; and the other R⁶ is methoxy; One R⁶ is isopropyl; and the other R⁶ is methoxy; or One R⁶ is isopropyl; and the other R⁶ is trifluoromethoxy.

In some embodiments, of the compound of formula AA,

the optionally substituted ring B is

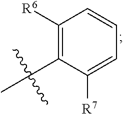

and R⁶ and R⁷ are one of the following combinations:
R⁶ is isopropyl; and R⁷ is methyl; R⁶ is isopropyl; and R⁷ is isopropyl; R⁶ is isopropyl; and R⁷ is trifluoromethyl; R⁶ is isopropyl; and R⁷ is cyclopropyl; R⁶ is isopropyl; and R⁷ is chloro; R⁶ is isopropyl; and R⁷ is fluoro; R⁶ is ethyl; and R⁷ is fluoro; R⁶ is isopropyl; and R⁷ is cyano; R⁶ is cyclopropyl; and R⁷ is cyclopropyl; R⁶ is cyclopropyl; and R⁷ is chloro; R⁶ is cyclopropyl; and R⁷ is fluoro; R⁶ is isopropyl; and R⁷ is methoxy; R⁶ is isopropyl; and R⁷ is trifluoromethoxy; R⁶ is chloro; and R⁷ is trifluoromethyl; R⁶ is chloro; and R⁷ is trifluoromethoxy; R⁷ is isopropyl; and R⁶ is methyl; R⁷ is isopropyl; and R⁶ is trifluoromethyl; R⁷ is isopropyl; and R⁶ is cyclopropyl; R⁷ is isopropyl; and R⁶ is chloro; R⁷ is ethyl; and R⁶ is fluoro; R⁷ is isopropyl; and R⁶ is cyano; R⁷ is cyclopropyl; and R⁶ is cyclopropyl; R⁷ is cyclopropyl; and R⁶ is chloro; R⁷ is cyclopropyl; and R⁶ is fluoro; R⁷ is isopropyl; and R⁶ is methoxy; R⁷ is isopropyl; and R⁶ is trifluoromethoxy; R⁷ is chloro; and R⁶ is trifluoromethyl; or R⁷ is chloro; and R⁶ is trifluoromethoxy.

In some embodiments, of the compound of formula AA,

the optionally substituted ring B is

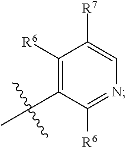

and R⁶ and R⁷ are one of the following combinations:
each R⁶ is independently C₁-C₆ alkyl, and R⁷ is C₁-C₆ alkyl optionally substituted with one or more halo; each R⁶ is independently C₁-C₆ alkyl and R⁷ is C₁-C₆ alkyl; each R⁶ is independently C₁-C₆ alkyl, and R⁷ is C₁-C₆ alkyl substituted with one or more halo; each R⁶ is independently C₁-C₆ alkyl, and R⁷ is C₃-C₇ cycloalkyl; each R⁶ is independently C₁-C₆ alkyl, and R⁷ is halo; each R⁶ is independently C₁-C₆ alkyl, and R⁷ is cyano; each R⁶ is independently C₃-C₇ cycloalkyl, and R⁷ is C₃-C₇ cycloalkyl; each R⁶ is independently C₃-C₇ cycloalkyl, and R⁷ is halo; each R⁶ is independently cyclopropyl, and R⁷ is halo; each R⁶ is independently C₁-C₆ alkyl, and R⁷ is C₁-C₆ alkoxy optionally substituted with one or more halo; each R⁶ is independently C₁-C₆ alkyl, and R⁷ is C₁-C₆ alkoxy; each R⁶ is independently C₁-C₆ alkyl, and R⁷ is C₁-C₆ alkoxy substituted with one or more halo; each R⁶ is independently halo, and R⁷ is C₁-C₆ haloalkyl; each R⁶ is independently halo, and R⁷ is C₁-C₆ haloalkoxy; each R⁶ is independently C₁-C₆ alkoxy; and R⁷ is halo; each R⁶ is independently C₁-C₆ alkoxy; and R⁷ is chloro; R⁷ is C₁-C₆ alkyl, and each R⁶ is independently C₁-C₆ alkyl optionally substituted with one or more halo; R⁷ is C₁-C₆ alkyl, and each R⁶ is independently C₁-C₆ alkyl substituted with one or more halo; R⁷ is C₁-C₆ alkyl, and each R⁶ is independently C₃-C₇ cycloalkyl; R⁷ is C₁-C₆ alkyl, and each R⁶ is independently halo; R⁷ is C₁-C₆ alkyl and each R⁶ is independently halo; R⁷ is C₁-C₆ alkyl, and R⁶ is cyano; R⁷ is C₃-C₇ cycloalkyl, and each R⁶ is independently C₃-C₇ cycloalkyl; R⁷ is C₃-C₇ cycloalkyl, and each R⁶ is independently halo; R⁷ is C₃-C₇ cycloalkyl and each R⁶ is independently halo; R⁷ is C₁-C₆ alkyl, and each R⁶ is independently C₁-C₆ alkoxy optionally substituted with one or more halo; R⁷ is C₁-C₆ alkyl, and each R⁶ is independently C₁-C₆ alkoxy; R⁷ is C₁-C₆ alkyl, and each R⁶ is independently C₁-C₆ alkoxy substituted with one or more halo; R⁷ is halo, and each R⁶ is independently C₁-C₆ haloalkyl; R⁷ is halo, and each R⁶ is independently C₁-C₆ haloalkoxy; R⁷ is C₁-C₆ alkoxy; and each R⁶ is independently halo; or R⁷ is C₁-C₆ alkoxy; and R⁶ is chloro.

In some embodiments, of the compound of formula AA,

the optionally substituted ring B is

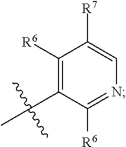

and R⁶ and R⁷ are one of the following combinations:
each R⁶ is isopropyl; and R⁷ is methyl; each R⁶ is isopropyl; and R⁷ is isopropyl; each R⁶ is isopropyl; and R⁷ is trifluoromethyl; each R⁶ is isopropyl; and R⁷ is cyclopropyl; each R⁶ is isopropyl; and R⁷ is chloro; each R⁶ is isopropyl; and R⁷ is fluoro; each R⁶ is ethyl; and R⁷ is fluoro; each R⁶ is isopropyl; and R⁷ is cyano; each R⁶ is cyclopropyl; and R⁷ is cyclopropyl; each R⁶ is cyclopropyl; and R⁷ is chloro; each R⁶ is cyclopropyl; and R⁷ is fluoro; each R⁶ is isopropyl; and R⁷ is methoxy; each R⁶ is isopropyl; and R⁷ is trifluoromethoxy; each R⁶ is chloro; and R⁷ is trifluoromethyl; each R⁶ is chloro; and R⁷ is trifluoromethoxy; R⁷ is isopropyl; and each R⁶ is methyl; R⁷ is isopropyl; and each R⁶ is trifluoromethyl; R⁷ is isopropyl; and each R⁶ is cyclopropyl; R⁷ is isopropyl; and each R⁶ is chloro; R⁷ is ethyl; and each R⁶ is fluoro; R⁷ is isopropyl; and each R⁶ is cyano; R⁷ is cyclopropyl; and each R⁶ is cyclopropyl; R⁷ is cyclopropyl; and each R⁶ is chloro; R⁷ is cyclopropyl; and each R⁶ is fluoro; R⁷ is isopropyl; and each R⁶ is methoxy; R⁷ is isopropyl; and each R⁶ is trifluoromethoxy; R⁷ is chloro; and each R⁶ is trifluoromethyl; R⁷ is chloro; and each R⁶ is trifluoromethoxy; or one R⁶ is isopropyl; the other R⁶ is trifluoromethyl; and R⁷ is chloro.

In some embodiments, of the compound of formula AA,

the optionally substituted ring B is

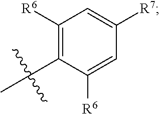

and R⁶ and R⁷ are one of the following combinations:
each R⁶ is independently C₁-C₆ alkyl, and R⁷ is C₁-C₆ alkyl optionally substituted with one or more halo; each R⁶ is independently C₁-C₆ alkyl and R⁷ is C₁-C₆ alkyl; each R⁶ is independently C₁-C₆ alkyl, and R⁷ is C₁-C₆ alkyl substituted with one or more halo; each R⁶ is independently C₁-C₆ alkyl, and R⁷ is C₃-C₇ cycloalkyl; each R⁶ is independently C₁-C₆ alkyl, and R⁷ is halo; each R⁶ is independently C₁-C₆ alkyl, and R⁷ is cyano; each R⁶ is independently C₃-C₇ cycloalkyl, and R⁷ is C₃-C₇ cycloalkyl; each R⁶ is independently C₃-C₇ cycloalkyl, and R⁷ is halo; each R⁶ is independently cyclopropyl and R⁷ is halo; each R⁶ is independently C₁-C₆ alkyl, and R⁷ is C₁-C₆ alkoxy optionally substituted with one or more halo; each R⁶ is independently C₁-C₆ alkyl, and R⁷ is C₁-C₆ alkoxy; each R⁶ is independently C₁-C₆ alkyl, and R⁷ is C₁-C₆ alkoxy substituted with one or more halo; each R⁶ is independently halo, and R⁷ is C₁-C₆ haloalkyl; each R⁶ is independently halo, and R⁷ is C₁-C₆ haloalkoxy; each R⁶ is independently C₁-C₆ alkoxy; and R⁷ is halo; each R⁶ is independently C₁-C₆ alkoxy; and R⁷ is chloro; R⁷ is C₁-C₆ alkyl, and each R⁶ is independently C₁-C₆ alkyl optionally substituted with one or more halo; R⁷ is C₁-C₆ alkyl, and each R⁶ is independently C₁-C₆ alkyl substituted with one or more halo; R⁷ is C₁-C₆ alkyl, and each R⁶ is independently C₃-C₇ cycloalkyl; R⁷ is C₁-C₆ alkyl, and each R⁶ is independently halo; R⁷ is C₁-C₆ alkyl and each R⁶ is independently halo; R⁷ is C₁-C₆ alkyl, and R⁶ is cyano; R⁷ is C₃-C₇ cycloalkyl, and each R⁶ is independently C₃-C₇ cycloalkyl; R⁷ is C₃-C₇ cycloalkyl, and each R⁶ is independently halo; R⁷ is C₃-C₇ cycloalkyl and each R⁶ is independently halo; R⁷ is C₁-C₆ alkyl, and each R⁶ is independently C₁-C₆, alkoxy optionally substituted with one or more halo; R⁷ is C₁-C₆ alkyl, and each R⁶ is independently C₁-C₆ alkoxy; R⁷ is C₁-C₆ alkyl, and each R⁶ is independently C₁-C₆ alkoxy substituted with one or more halo; R⁷ is halo, and each R⁶ is independently C₁-C₆ haloalkyl; R⁷ is halo, and each R⁶ is independently C₁-C₆ haloalkoxy; R⁷ is C₁-C₆ alkoxy; and each R⁶ is independently halo; or R⁷ is C₁-C₆ alkoxy; and R⁶ is chloro.

In some embodiments, of the compound of formula AA,

the optionally substituted ring B is

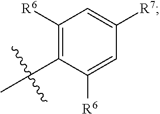

and R⁶ and R⁷ are one of the following combinations:
each R⁶ is isopropyl; and R⁷ is methyl; each R⁶ is isopropyl; and R⁷ is isopropyl; each R⁶ is isopropyl; and R⁷ is trifluoromethyl; each R⁶ is isopropyl; and R⁷ is cyclopropyl; each R⁶ is isopropyl; and R⁷ is chloro; each R⁶ is isopropyl; and R⁷ is fluoro; each R⁶ is ethyl; and R⁷ is fluoro; each R⁶ is isopropyl; and R⁷ is cyano; each R⁶ is cyclopropyl; and R⁷ is cyclopropyl; each R⁶ is cyclopropyl; and R⁷ is chloro; each R⁶ is cyclopropyl; and R⁷ is fluoro; each R⁶ is isopropyl; and R⁷ is methoxy; each R⁶ is isopropyl; and R⁷ is trifluoromethoxy; each R⁶ is chloro; and R⁷ is trifluoromethyl; each R⁶ is chloro; and R⁷ is trifluoromethoxy; R⁷ is isopropyl; and each R⁶ is methyl; R⁷ is isopropyl; and each R⁶ is trifluoromethyl; R⁷ is isopropyl; and each R⁶ is cyclopropyl; R⁷ is isopropyl; and each R⁶ is chloro; R⁷ is ethyl; and each R⁶ is fluoro; R⁷ is isopropyl; and each R⁶ is cyano; R⁷ is cyclopropyl; and each R⁶ is cyclopropyl; R⁷ is cyclopropyl; and each R⁶ is chloro; R⁷ is cyclopropyl; and each R⁶ is fluoro; R⁷ is isopropyl; and each R⁶ is methoxy; R⁷ is isopropyl; and each R⁶ is trifluoromethoxy; R⁷ is chloro; and each R⁶ is trifluoromethyl; R⁷ is chloro; and each R⁶ is trifluoromethoxy; or one R⁶ is isopropyl; the other R⁶ is trifluoromethyl; and R⁷ is chloro.

In some embodiments, of the compound of formula AA,

the optionally substituted ring B is

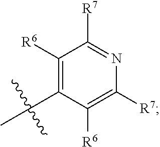

and R⁶ and R⁷ are one of the following combinations:
each R⁶ is independently C₁-C₆ alkyl, and each R⁷ is independently C₁-C₆ alkyl optionally substituted with one or more halo; each R⁶ is independently C₁-C₆ alkyl and each R⁷ is independently C₁-C₆ alkyl; each R⁶ is independently C₁-C₆ alkyl, and each R⁷ is independently C₁-C₆ alkyl substituted with one or more halo; each R⁶ is independently C₁-C₆ alkyl, and each R⁷ is independently C₃-C₇ cycloalkyl; each R⁶ is independently C₁-C₆ alkyl, and each R⁷ is independently halo; each R⁶ is independently C₁-C₆ alkyl, and R⁷ is cyano; each R⁶ is independently C₃-C₇ cycloalkyl, and each R⁷ is independently C₃-C₇ cycloalkyl; each R⁶ is independently C₃-C₇ cycloalkyl, and each R⁷ is independently halo; each R⁶ is independently cyclopropyl and each R⁷ is independently halo; each R⁶ is independently C₁-C₆ alkyl, and each R⁷ is independently C₁-C₆ alkoxy optionally substituted with one or more halo; each R⁶ is independently C₁-C₆ alkyl, and each R⁷ is independently C₁-C₆ alkoxy; each R⁶ is independently C₁-C₆ alkyl, and each R⁷ is independently C₁-C₆ alkoxy substituted with one or more halo; each R⁶ is independently halo, and each R⁷ is independently C₁-C₆ haloalkyl; each R⁶ is independently halo, and each R⁷ is independently C₁-C₆ haloalkoxy; each R⁶ is independently C₁-C₆ alkoxy; and each R⁷ is independently halo; each R⁶ is independently C₁-C₆ alkoxy; and R⁷ is chloro; each R⁷ is independently C₁-C₆ alkyl, and each R⁶ is independently C₁-C₆ alkyl optionally substituted with one or more halo; each R⁷ is independently C₁-C₆ alkyl, and each R⁶ is independently C₁-C₆ alkyl substituted with one or more halo; each R⁷ is independently C₁-C₆ alkyl, and each R⁶ is independently C₃-C₇ cycloalkyl; each R⁷ is independently C₁-C₆ alkyl, and each R⁶ is independently halo; each R⁷ is independently C₁-C₆ alkyl and each R⁶ is independently halo; each R⁷ is independently C₁-C₆ alkyl, and R⁶ is cyano; each R⁷ is independently C₃-C₇ cycloalkyl, and each R⁶ is independently C₃-C₇ cycloalkyl; each R⁷ is independently C₃-C₇ cycloalkyl, and each R⁶ is independently halo; each R⁷ is independently C₃-C₇ cycloalkyl and each R⁶ is independently halo; each R⁷ is independently C₁-C₆ alkyl, and each R⁶ is independently C₁-C₆ alkoxy optionally substituted with one or more halo; each R⁷ is independently C₁-C₆ alkyl, and each R⁶ is independently C₁-C₆ alkoxy; each R⁷ is independently C₁-C₆ alkyl, and each R⁶ is independently C₁-C₆ alkoxy substituted with one or more halo; each R⁷ is independently halo, and each R⁶ is independently C₁-C₆ haloalkyl; each R⁷ is independently halo, and each R⁶ is independently C₁-C₆ haloalkoxy; each R⁷ is independently C₁-C₆ alkoxy; and each R⁶ is independently halo; each R⁷ is independently C₁-C₆ alkoxy; and R⁶ is chloro; or two pairs, each of one R⁶ and one R⁷, are on adjacent atoms, and each pair of one R⁶ and one R⁷ taken together with the atoms connecting them form a C₄-C₈ aliphatic carbocyclic ring.

In some embodiments, of the compound of formula AA,

the optionally substituted ring B is

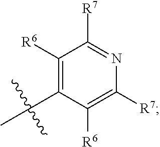

and R⁶ and R⁷ are one of the following combinations:
each R⁶ is isopropyl; and each R⁷ is methyl; each R⁶ is isopropyl; and each R⁷ is isopropyl; each R⁶ is isopropyl; and each R⁷ is trifluoromethyl; each R⁶ is isopropyl; and each R⁷ is cyclopropyl; each R⁶ is isopropyl; and each R⁷ is chloro; each R⁶ is isopropyl; and each R⁷ is fluoro; each R⁶ is ethyl; and each R⁷ is fluoro; each R⁶ is isopropyl; and each R⁷ is cyano; each R⁶ is cyclopropyl; and each R⁷ is cyclopropyl; each R⁶ is cyclopropyl; and each R⁷ is chloro; each R⁶ is cyclopropyl; and each R⁷ is fluoro; each R⁶ is isopropyl; and each R⁷ is methoxy; each R⁶ is isopropyl; and each R⁷ is trifluoromethoxy; each R⁶ is chloro; and each R⁷ is trifluoromethyl; each R⁶ is chloro; and each R⁷ is trifluoromethoxy; each R⁷ is isopropyl; and each R⁶ is methyl; each R⁷ is isopropyl; and each R⁶ is trifluoromethyl; each R⁷ is isopropyl; and each R⁶ is cyclopropyl; each R⁷ is isopropyl; and each R⁶ is chloro; each R⁷ is ethyl; and each R⁶ is fluoro; each R⁷ is isopropyl; and each R⁶ is cyano; each R⁷ is cyclopropyl; and each R⁶ is cyclopropyl; each R⁷ is cyclopropyl; and each R⁶ is chloro; each R⁷ is cyclopropyl; and each R⁶ is fluoro; each R⁷ is isopropyl; and each R⁶ is methoxy; each R⁷ is isopropyl; and each R⁶ is trifluoromethoxy; each R⁷ is chloro; and each R⁶ is trifluoromethyl; each R⁷ is chloro; and each R⁶ is trifluoromethoxy; one R⁶ is isopropyl; the other R⁶ is trifluoromethyl; and each R⁷ is chloro; each R⁶ is isopropyl; one R⁷ is fluoro; and the other R⁷ is cyano; or two pairs, each of one R⁶ and one R⁷, are on adjacent atoms, and each pair of one R⁶ and one R⁷ taken together with the atoms connecting them form a C₅ aliphatic carbocyclic ring.

In some embodiments, of the compound of formula AA,

the optionally substituted ring B is

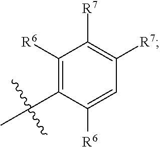

and R⁶ and R⁷ are one of the following combinations:
each R⁶ is independently C₁-C₆ alkyl, and each R⁷ is independently C₁-C₆ alkyl optionally substituted with one or more halo; each R⁶ is independently C₁-C₆ alkyl and each R⁷ is independently C₁-C₆ alkyl; each R⁶ is independently C₁-C₆ alkyl, and each R⁷ is independently C₁-C₆ alkyl substituted with one or more halo; each R⁶ is independently C₁-C₆ alkyl, and each R⁷ is independently C₃-C₇ cycloalkyl; each R⁶ is independently C₁-C₆ alkyl, and each R⁷ is independently halo; each R⁶ is independently C₁-C₆ alkyl, and R⁷ is cyano; each R⁶ is independently C₃-C₇ cycloalkyl, and each R⁷ is independently C₃-C₇ cycloalkyl; each R⁶ is independently C₃-C₇ cycloalkyl, and each R⁷ is independently halo; each R⁶ is independently cyclopropyl and each R⁷ is independently halo; each R⁶ is independently C₁-C₆ alkyl, and each R⁷ is independently C₁-C₆ alkoxy optionally substituted with one or more halo; each R⁶ is independently C₁-C₆ alkyl, and each R⁷ is independently C₁-C₆ alkoxy; each R⁶ is independently C₁-C₆ alkyl, and each R⁷ is independently C₁-C₆ alkoxy substituted with one or more halo; each R⁶ is independently halo, and each R⁷ is independently C₁-C₆ haloalkyl; each R⁶ is independently halo, and each R⁷ is independently C₁-C₆ haloalkoxy; each R⁶ is independently C₁-C₆ alkoxy; and each R⁷ is independently halo; each R⁶ is independently C₁-C₆ alkoxy; and R⁷ is chloro; each R⁷ is independently C₁-C₆ alkyl, and each R⁶ is independently C₁-C₆ alkyl optionally substituted with one or more halo; each R⁷ is independently C₁-C₆ alkyl, and each R⁶ is independently C₁-C₆ alkyl substituted with one or more halo; each R⁷ is independently C₁-C₆ alkyl, and each R⁶ is independently C₃-C₇ cycloalkyl; each R⁷ is independently C₁-C₆ alkyl, and each R⁶ is independently halo; each R⁷ is independently C₁-C₆ alkyl and each R⁶ is independently halo; each R⁷ is independently C₁-C₆ alkyl, and R⁶ is cyano; each R⁷ is independently C₃-C₇ cycloalkyl, and each R⁶ is independently C₃-C₇ cycloalkyl; each R⁷ is independently C₃-C₇ cycloalkyl, and each R⁶ is independently halo; each R⁷ is independently C₃-C₇ cycloalkyl and each R⁶ is independently halo; each R⁷ is independently C₁-C₆ alkyl, and each R⁶ is independently C₁-C₆ alkoxy optionally substituted with one or more halo; each R⁷ is independently C₁-C₆ alkyl, and each R⁶ is independently C₁-C₆ alkoxy; each R⁷ is independently C₁-C₆ alkyl, and each R⁶ is independently C₁-C₆, alkoxy substituted with one or more halo; each R⁷ is independently halo, and each R⁶ is independently C₁-C₆ haloalkyl; each R⁷ is independently halo, and each R⁶ is independently C₁-C₆ haloalkoxy; each R⁷ is independently C₁-C₆ alkoxy; and each R⁶ is independently halo; or each R⁷ is independently C₁-C₆ alkoxy; and R⁶ is chloro.

In some embodiments, of the compound of formula AA,

the optionally substituted ring B is

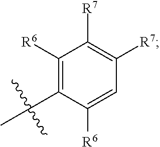

and R⁶ and R⁷ are one of the following combinations:
each R⁶ is isopropyl; and each R⁷ is methyl; each R⁶ is isopropyl; and each R⁷ is isopropyl; each R⁶ is isopropyl; and each R⁷ is trifluoromethyl; each R⁶ is isopropyl; and each R⁷ is cyclopropyl; each R⁶ is isopropyl; and each R⁷ is chloro; each R⁶ is isopropyl; and each R⁷ is fluoro; each R⁶ is ethyl; and each R⁷ is fluoro; each R⁶ is isopropyl; and each R⁷ is cyano; each R⁶ is cyclopropyl; and each R⁷ is cyclopropyl; each R⁶ is cyclopropyl; and each R⁷ is chloro; each R⁶ is cyclopropyl; and each R⁷ is fluoro; each R⁶ is isopropyl; and each R⁷ is methoxy; each R⁶ is isopropyl; and each R⁷ is trifluoromethoxy; each R⁶ is chloro; and each R⁷ is trifluoromethyl; each R⁶ is chloro; and each R⁷ is trifluoromethoxy; each R⁷ is isopropyl; and each R⁶ is methyl; each R⁷ is isopropyl; and each R⁶ is trifluoromethyl; each R⁷ is isopropyl; and each R⁶ is cyclopropyl; each R⁷ is isopropyl; and each R⁶ is chloro; each R⁷ is ethyl; and each R⁶ is fluoro; each R⁷ is isopropyl; and each R⁶ is cyano; each R⁷ is cyclopropyl; and each R⁶ is cyclopropyl; each R⁷ is cyclopropyl; and each R⁶ is chloro; each R⁷ is cyclopropyl; and each R⁶ is fluoro; each R⁷ is isopropyl; and each R⁶ is methoxy; each R⁷ is isopropyl; and each R⁶ is trifluoromethoxy; each R⁷ is chloro; and each R⁶ is trifluoromethyl; each R⁷ is chloro; and each R⁶ is trifluoromethoxy; one R⁶ is isopropyl; the other R⁶ is trifluoromethyl; and R⁷ is chloro; or R⁶ is isopropyl; one R⁷ is fluoro; and the other R⁷ is cyano.

In some embodiments, of the compound of formula AA,

the optionally substituted ring B is

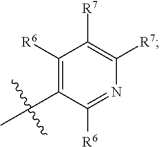

and R⁶ and R⁷ are one of the following combinations:
each R⁶ is independently C₁-C₆ alkyl, and each R⁷ is independently C₁-C₆ alkyl optionally substituted with one or more halo; each R⁶ is independently C₁-C₆ alkyl and each R⁷ is independently C₁-C₆ alkyl; each R⁶ is independently C₁-C₆ alkyl, and each R⁷ is independently C₁-C₆ alkyl substituted with one or more halo; each R⁶ is independently C₁-C₆, alkyl, and each R⁷ is independently C₃-C₇ cycloalkyl; each R⁶ is independently C₁-C₆ alkyl, and each R⁷ is independently halo; each R⁶ is independently C₁-C₆ alkyl, and R⁷ is cyano; each R⁶ is independently C₃-C₇ cycloalkyl, and each R⁷ is independently C₃-C₇ cycloalkyl; each R⁶ is independently C₃-C₇ cycloalkyl, and each R⁷ is independently halo; each R⁶ is independently cyclopropyl and each R⁷ is independently halo; each R⁶ is independently C₁-C₆ alkyl, and each R⁷ is independently C₁-C₆ alkoxy optionally substituted with one or more halo; each R⁶ is independently C₁-C₆ alkyl, and each R⁷ is independently C₁-C₆ alkoxy; each R⁶ is independently C₁-C₆ alkyl, and each R⁷ is independently C₁-C₆ alkoxy substituted with one or more halo; each R⁶ is independently halo, and each R⁷ is independently C₁-C₆ haloalkyl; each R⁶ is independently halo, and each R⁷ is independently C₁-C₆ haloalkoxy; each R⁶ is independently C₁-C₆ alkoxy; and each R⁷ is independently halo; each R⁶ is independently C₁-C₆ alkoxy; and R⁷ is chloro; each R⁷ is independently C₁-C₆ alkyl, and each R⁶ is independently C₁-C₆ alkyl optionally substituted with one or more halo; each R⁷ is independently C₁-C₆ alkyl, and each R⁶ is independently C₁-C₆ alkyl substituted with one or more halo; each R⁷ is independently C₁-C₆ alkyl, and each R⁶ is independently C₃-C₇ cycloalkyl; each R⁷ is independently C₁-C₆ alkyl, and each R⁶ is independently halo; each R⁷ is independently C₁-C₆ alkyl and each R⁶ is independently halo; each R⁷ is independently C₁-C₆ alkyl, and R⁶ is cyano; each R⁷ is independently C₃-C₇ cycloalkyl, and each R⁶ is independently C₃-C₇ cycloalkyl; each R⁷ is independently C₃-C₇ cycloalkyl, and each R⁶ is independently halo; each R⁷ is independently C₃-C₇ cycloalkyl and each R⁶ is independently halo; each R⁷ is independently C₁-C₆ alkyl, and each R⁶ is independently C₁-C₆ alkoxy optionally substituted with one or more halo; each R⁷ is independently C₁-C₆ alkyl, and each R⁶ is independently C₁-C₆ alkoxy; each R⁷ is independently C₁-C₆ alkyl, and each R⁶ is independently C₁-C₆ alkoxy substituted with one or more halo; each R⁷ is independently halo, and each R⁶ is independently C₁-C₆ haloalkyl; each R⁷ is independently halo, and each R⁶ is independently C₁-C₆ haloalkoxy; each R⁷ is independently C₁-C₆ alkoxy; and each R⁶ is independently halo; or each R⁷ is independently C₁-C₆ alkoxy; and R⁶ is chloro.

In some embodiments, of the compound of formula AA,

the optionally substituted ring B is

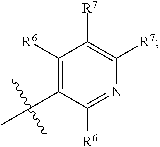

and R⁶ and R⁷ are one of the following combinations:
each R⁶ is isopropyl; and each R⁷ is methyl; each R⁶ is isopropyl; and each R⁷ is isopropyl; each R⁶ is isopropyl; and each R⁷ is trifluoromethyl; each R⁶ is isopropyl; and each R⁷ is cyclopropyl; each R⁶ is isopropyl; and each R⁷ is chloro; each R⁶ is isopropyl; and each R⁷ is fluoro; each R⁶ is ethyl; and each R⁷ is fluoro; each R⁶ is isopropyl; and each R⁷ is cyano; each R⁶ is cyclopropyl; and each R⁷ is cyclopropyl; each R⁶ is cyclopropyl; and each R⁷ is chloro; each R⁶ is cyclopropyl; and each R⁷ is fluoro; each R⁶ is isopropyl; and each R⁷ is methoxy; each R⁶ is isopropyl; and each R⁷ is trifluoromethoxy; each R⁶ is chloro; and each R⁷ is trifluoromethyl; each R⁶ is chloro; and each R⁷ is trifluoromethoxy; each R⁷ is isopropyl; and each R⁶ is methyl; each R⁷ is isopropyl; and each R⁶ is trifluoromethyl; each R⁷ is isopropyl; and each R⁶ is cyclopropyl; each R⁷ is isopropyl; and each R⁶ is chloro; each R⁷ is ethyl; and each R⁶ is fluoro; each R⁷ is isopropyl; and each R⁶ is cyano; each R⁷ is cyclopropyl; and each R⁶ is cyclopropyl; each R⁷ is cyclopropyl; and each R⁶ is chloro; each R⁷ is cyclopropyl; and each R⁶ is fluoro; each R⁷ is isopropyl; and each R⁶ is methoxy; each R⁷ is isopropyl; and each R⁶ is trifluoromethoxy; each R⁷ is chloro; and each R⁶ is trifluoromethyl; each R⁷ is chloro; and each R⁶ is trifluoromethoxy; one R⁶ is isopropyl; the other R⁶ is trifluoromethyl; and each R⁷ is chloro; or each R⁶ is isopropyl; one R⁷ is fluoro; and the other R⁷ is cyano.

In some embodiments, of the compound of formula AA,

the optionally substituted ring B is

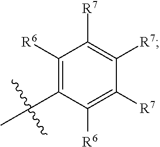

and R⁶ and R⁷ are one of the following combinations:
each R⁶ is independently C₁-C₆ alkyl, and R⁷ is C₁-C₆ alkyl optionally substituted with one or more halo; each R⁶ is independently C₁-C₆ alkyl and R⁷ is C₁-C₆ alkyl; each R⁶ is independently C₁-C₆ alkyl, and R⁷ is C₁-C₆ alkyl substituted with one or more halo; each R⁶ is independently C₁-C₆ alkyl, and R⁷ is C₃-C₇ cycloalkyl; each R⁶ is independently C₁-C₆ alkyl, and R⁷ is halo; each R⁶ is independently C₁-C₆ alkyl, and R⁷ is cyano; each R⁶ is independently C₃-C₇ cycloalkyl, and R⁷ is C₃-C₇ cycloalkyl; each R⁶ is independently C₃-C₇ cycloalkyl, and R⁷ is halo; each R⁶ is independently cyclopropyl and R⁷ is halo; each R⁶ is independently C₁-C₆ alkyl, and R⁷ is C₁-C₆ alkoxy optionally substituted with one or more halo; each R⁶ is independently C₁-C₆ alkyl, and R⁷ is C₁-C₆ alkoxy; each R⁶ is independently C₁-C₆ alkyl, and R⁷ is C₁-C₆ alkoxy substituted with one or more halo; each R⁶ is independently halo, and R⁷ is C₁-C₆ haloalkyl; each R⁶ is independently halo, and R⁷ is C₁-C₆ haloalkoxy; each R⁶ is independently C₁-C₆ alkoxy; and R⁷ is halo; each R⁶ is independently C₁-C₆ alkoxy; and R⁷ is chloro; R⁷ is C₁-C₆ alkyl, and each R⁶ is independently C₁-C₆ alkyl optionally substituted with one or more halo; R⁷ is C₁-C₆ alkyl, and each R⁶ is independently C₁-C₆ alkyl substituted with one or more halo; R⁷ is C₁-C₆ alkyl, and each R⁶ is independently C₃-C₇ cycloalkyl; R⁷ is C₁-C₆ alkyl, and each R⁶ is independently halo; R⁷ is C₁-C₆ alkyl and each R⁶ is independently halo; R⁷ is C₁-C₆ alkyl, and R⁶ is cyano; R⁷ is C₃-C₇ cycloalkyl, and each R⁶ is independently C₃-C₇ cycloalkyl; R⁷ is C₃-C₇ cycloalkyl, and each R⁶ is independently halo; R⁷ is C₃-C₇ cycloalkyl and each R⁶ is independently halo; R⁷ is C₁-C₆ alkyl, and each R⁶ is independently C₁-C₆, alkoxy optionally substituted with one or more halo; R⁷ is C₁-C₆ alkyl, and each R⁶ is independently C₁-C₆ alkoxy; R⁷ is C₁-C₆ alkyl, and each R⁶ is independently C₁-C₆ alkoxy substituted with one or more halo; R⁷ is halo, and each R⁶ is independently C₁-C₆ haloalkyl; R⁷ is halo, and each R⁶ is independently C₁-C₆ haloalkoxy; R⁷ is C₁-C₆ alkoxy; and each R⁶ is independently halo; R⁷ is C₁-C₆ alkoxy; and R⁶ is chloro; two pairs, each of one R⁶ and one R⁷, are on adjacent atoms, and each pair of one R⁶ and one R⁷ taken together with the atoms connecting them form a C₄-C₈ aliphatic carbocyclic ring; and one R⁷ is halo; or two pairs, each of one R⁶ and one R⁷, are on adjacent atoms, and each pair of one R⁶ and one R⁷ taken together with the atoms connecting them form a C₄-C₈ aliphatic carbocyclic ring; and one R⁷ is cyano.

In some embodiments, of the compound of formula AA,

the optionally substituted ring B is

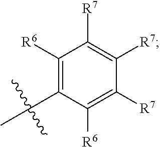

and R⁶ and R⁷ are one of the following combinations:
each R⁶ is isopropyl; and each R⁷ is methyl; each R⁶ is isopropyl; and each R⁷ is isopropyl; each R⁶ is isopropyl; and each R⁷ is trifluoromethyl; each R⁶ is isopropyl; and each R⁷ is cyclopropyl; each R⁶ is isopropyl; and each R⁷ is chloro; each R⁶ is isopropyl; and each R⁷ is fluoro; each R⁶ is ethyl; and each R⁷ is fluoro; each R⁶ is isopropyl; and each R⁷ is cyano; each R⁶ is cyclopropyl; and each R⁷ is cyclopropyl; each R⁶ is cyclopropyl; and each R⁷ is chloro; each R⁶ is cyclopropyl; and each R⁷ is fluoro; each R⁶ is isopropyl; and each R⁷ is methoxy; each R⁶ is isopropyl; and each R⁷ is trifluoromethoxy; each R⁶ is chloro; and each R⁷ is trifluoromethyl; each R⁶ is chloro; and each R⁷ is trifluoromethoxy; each R⁷ is isopropyl; and each R⁶ is methyl; each R⁷ is isopropyl; and each R⁶ is trifluoromethyl; each R⁷ is isopropyl; and each R⁶ is cyclopropyl; each R⁷ is isopropyl; and each R⁶ is chloro; each R⁷ is ethyl; and each R⁶ is fluoro; each R⁷ is isopropyl; and each R⁶ is cyano; each R⁷ is cyclopropyl; and each R⁶ is cyclopropyl; each R⁷ is cyclopropyl; and each R⁶ is chloro; each R⁷ is cyclopropyl; and each R⁶ is fluoro; each R⁷ is isopropyl; and each R⁶ is methoxy; each R⁷ is isopropyl; and each R⁶ is trifluoromethoxy; each R⁷ is chloro; and each R⁶ is trifluoromethyl; each R⁷ is chloro; and each R⁶ is trifluoromethoxy; each R⁶ is isopropyl; two R⁷ are fluoro; and one R⁷ is chloro; two pairs, each of one R⁶ and one R⁷, are on adjacent atoms, and each pair of one R⁶ and one R⁷ taken together with the atoms connecting them form a C₅ aliphatic carbocyclic ring; and one R⁷ is chloro; or two pairs, each of one R⁶ and one R⁷, are on adjacent atoms, and each pair of one R⁶ and one R⁷ taken together with the atoms connecting them form a C₅ aliphatic carbocyclic ring; and one R⁷ is fluoro.

In some embodiments, the compound of formula AA is a compound of Formula Va

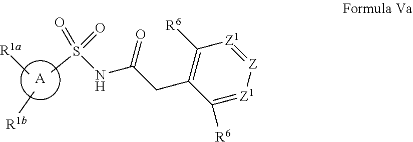

wherein
A is thiazolyl;
R^{1a} is a C₁-C₆ alkyl substituted with one or more hydroxy or —OSi(R¹³)₃;
R^{1b} is a C₁-C₆ alkyl substituted with one or more hydroxy;

### Z is N, CH, or CR⁷;

each R⁶ is independently hydrogen, C₁-C₆ alkoxy, halo, C₁-C₆ haloalkyl, C₃-C₇ cycloalkyl, or C₁-C₆ alkyl optionally substituted with hydroxy;
each Z¹ is independently N, CH or CR⁷, each R⁷ is independently hydrogen, C₁-C₆ alkoxy, halo, C₁-C₆ haloalkyl, CN, C₁-C₆ haloalkoxy, C₃-C₇ cycloalkyl, or C₁-C₆ alkyl optionally substituted with hydroxy;
or at least one pair of R⁶ and R⁷ in adjacent positions, taken together with the carbon atoms connecting them, form a four-membered to seven-membered carbocyclic or heterocyclic ring containing 1 or 2 heteroatoms independently selected from O, N, and S; and wherein the four-membered to seven-membered carbocyclic or heterocyclic ring is optionally independently substituted with one or more substituents selected from H, F, C₁-C₆ alkyl, C₁-C₆, alkoxy, NR⁸R⁹, oxo, and ═NR¹⁰.

In some embodiments of the compound of formula Va, A is 2-thiazolyl. In some embodiments of the compound of formula Va, A is 4-thiazolyl. In some embodiments of the compound of formula Va, A is 5-thiazolyl.

In some embodiments, the compound of Formula Va is a compound of Formula Va-i:

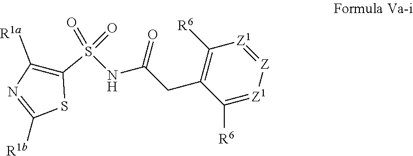

In some embodiments, the compound of Formula Va is a compound of Formula Va-ii:

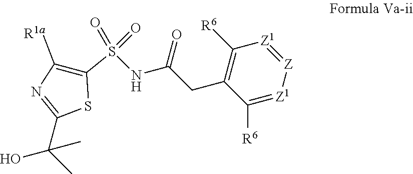

In some embodiments, the compound of Formula Va is a compound of Formula Va-iii:

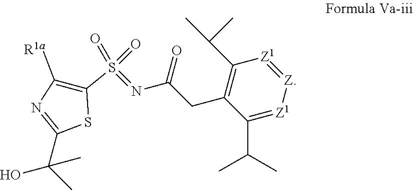

In some embodiments, the compound of Formula Va is a compound of Formula Va-iv:

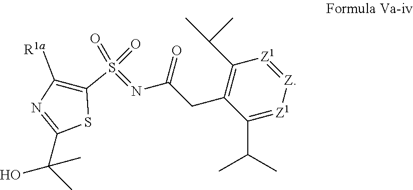

wherein Z¹ is CH or CR⁷; and
R^{1a} is an unbranched C₁-C₆ alkyl substituted with one hydroxy.

In some embodiments of the compound of formula Va, Va-i, Va-ii, and Va-iii, Z is N.

In some embodiments of the compound of formula Va, Va-i, Va-ii, Va-iii, and Va-iv, Z is CR⁷.

In some embodiments of the compound of formula Va, Va-i, Va-ii, Va-iii, and Va-iv, Z is CH.

In some embodiments of the compound of formula Va, Va-i, Va-ii, Va-iii, and Va-vi, R^{1a} is hydroxymethyl. In some embodiments of the compound of formula Va, Va-i, Va-ii, Va-iii, and Va-vi, R^{1a} is hydroxyethyl. In some embodiments of the compound of formula Va, Va-i, Va-ii, Va-iii, and Va-vi, R^{1a} is 3-hydroxy-1-propyl. In some embodiments of the compound of formula Va, Va-i, Va-ii, and Va-iii, R^{1a} is 2-hydroxy-2-propyl. In some embodiments of the compound of formula Va, Va-i, Va-ii, and Va-iii, R^{1a} is 3-hydroxy-2-propyl. In some embodiments of the compound of formula Va, Va-i, Va-ii, and Va-iii, R^{1a} is 1-hydroxy-1-propyl. In some embodiments of the compound of formula Va, Va-i, Va-ii, and Va-iii, R^{1a} is 2-hydroxy-1-propyl. In some embodiments of the compound of formula Va, Va-i, Va-ii, and Va-iii, R^{1a} is hydroxybutyl. In some embodiments of the compound of formula Va, Va-i, Va-ii, and Va-iii, R^{1a} is hydroxypentyl. In some embodiments of the compound of formula Va, Va-i, Va-ii, and Va-iii, R^{1a} is hydroxyhexyl. In some embodiments of the compound of formula Va, Va-i, Va-ii, and Va-iii, R^{1a} is an unbranched C₁-C₆ alkyl substituted with one hydroxy. In some embodiments of the compound of formula Va, Va-i, Va-ii, and Va-iii, R^{1a} is a branched C₁-C₆ alkyl substituted with one hydroxy.

In some embodiments of the compound of formula Va and Va-i, R^{1b} is hydroxymethyl. In some embodiments of the compound of formula Va and Va-i, R^{1b} is hydroxyethyl. In some embodiments of the compound of formula Va and Va-i, R^{1b} is 2-hydroxy-2-propyl. In some embodiments of the compound of formula Va and Va-i, R^{1b} is 3-hydroxy-2-propyl. In some embodiments of the compound of formula Va and Va-i, R^{1b} is 1-hydroxy-1-propyl. In some embodiments of the compound of formula Va and Va-i, R^{1b} is 2-hydroxy-1-propyl. In some embodiments of the compound of formula Va and Va-i, R^{1b} is hydroxybutyl. In some embodiments of the compound of formula Va and Va-i, R^{1b} is hydroxypentyl. In some embodiments of the compound of formula Va and Va-i, R^{1b} is hydroxyhexyl. In some embodiments of the compound of formula Va and Va-i, R^{1b} is an unbranched C₁-C₆ alkyl substituted with one hydroxy. In some embodiments of the compound of formula Va and Va-i, R^{1b} is a branched C₁-C₆ alkyl substituted with one hydroxy.

In some embodiments of the compound of formula Va, Va-i, Va-ii, Va-iii, and Va-iv, each Z¹ is CH. In some embodiments of the compound of formula Va, Va-i, Va-ii, Va-iii, and Va-iv, one Z¹ is CH and the other Z¹ is CR⁷. In some embodiments of the compound of formula Va, Va-i, Va-ii, Va-iii, and Va-iv, each Z¹ is CR⁷. In some embodiments of the compound of formula Va, Va-i, Va-ii, Va-iii, and Va-iv, Z is CR⁷ wherein R⁷ is CN. In some embodiments of the compound of formula Va, Va-i, Va-ii, Va-iii, and Va-iv, Z is CR⁷ wherein R⁷ is halo (e.g., F). In some embodiments of the compound of formula Va, Va-i, Va-ii, Va-iii, and Va-iv, Z is CR⁷ wherein R⁷ is CO₂C₁-C₆ alkyl. In some embodiments of the compound of formula Va, Va-i, Va-ii, Va-iii, and Va-iv, Z is CR⁷ wherein R⁷ is CONR¹¹R¹²; In some embodiments of the compound of formula Va, Va-i, Va-ii, Va-iii, and Va-iv, Z is CR⁷ wherein R⁷ is C₁-C₆ alkyl. In some embodiments of the compound of formula Va, Va-i, Va-ii, Va-iii, and Va-iv, Z is CR⁷ wherein R⁷ is C₁-C₆ alkoxy; In some embodiments of the compound of formula Va, Va-i, Va-ii, Va-iii, and Va-iv, Z is CR⁷ wherein R⁷ is C₁-C₆ haloalkyl. In some embodiments of the compound of formula Va, Va-i, Va-ii, Va-iii, and Va-iv, each R⁷ that is meta to the CR⁴R⁵ group is hydrogen. In some embodiments of the compound of formula Va, Va-i, Va-ii, Va-iii, and Va-iv, each R⁷ that is meta to the CR⁴R⁵ group is C₁-C₆, alkoxy. In some embodiments of the compound of formula Va, Va-i, Va-ii, Va-iii, and Va-iv, each R⁷ that is meta to the CR⁴R⁵ group is halo. In some embodiments of the compound of formula Va, Va-i, Va-ii, Va-iii, and Va-iv, each R⁷ that is meta to the CR⁴R⁵ group is fluoro. In some embodiments of the compound of formula Va, Va-i, Va-ii, Va-iii, and Va-iv, each R⁷ that is meta to the CR⁴R⁵ group is C₁-C₆ haloalkyl. In some embodiments of the compound of formula Va, Va-i, Va-ii, Va-iii, and Va-iv, each R⁷ that is meta to the CR⁴R⁵ group is CN. In some embodiments of the compound of formula Va, Va-i, Va-ii, Va-iii, and Va-iv, each R⁷ that is meta to the CR⁴R⁵ group is C₃-C₇ cycloalkyl. In some embodiments of the compound of formula Va, Va-i, Va-ii, Va-iii, and Va-iv, each R⁷ that is meta to the CR⁴R⁵ group is C₁-C₆ alkyl optionally substituted with hydroxyl. In some embodiments of the compound of formula Va, Va-i, Va-ii, Va-iii, and Va-iv, each R⁷ that is meta to the CR⁴R⁵ group is unsubstituted C₁-C₆ alkyl. In some embodiments of the compound of formula Va, Va-i, Va-ii, Va-iii, and Va-iv, one R⁷ that is meta to the CR⁴R⁵ group is hydrogen and is different from the other R⁷ that is meta to the CR⁴R⁵ group. In some embodiments of the compound of formula Va, Va-i, Va-ii, Va-iii, and Va-iv, one R⁷ that is meta to the CR⁴R⁵ group is C₁-C₆, alkoxy and is different from the other R⁷ that is meta to the CR⁴R⁵ group. In some embodiments of the compound of formula Va, Va-i, Va-ii, Va-iii, and Va-iv, one R⁷ that is meta to the CR⁴R⁵ group is halo and is different from the other R⁷ that is meta to the CR⁴R⁵ group. In some embodiments of the compound of formula Va, Va-i, Va-ii, Va-iii, and Va-iv, one R⁷ that is meta to the CR⁴R⁵ group is C₁-C₆ haloalkyl and is different from the other R⁷ that is meta to the CR⁴R⁵ group. In some embodiments of the compound of formula Va, Va-i, Va-ii, Va-iii, and Va-iv, one R⁷ that is meta to the CR⁴R⁵ group is CN and is different from the other R⁷ that is meta to the CR⁴R⁵ group. In some embodiments of the compound of formula Va, Va-i, Va-ii, Va-iii, and Va-iv, one R⁷ that is meta to the CR⁴R⁵ group is C₃-C₇ cycloalkyl and is different from the other R⁷ that is meta to the CR⁴R⁵ group. In some embodiments of the compound of formula Va, Va-i, Va-ii, Va-iii, and Va-iv, one R⁷ that is meta to the CR⁴R⁵ group is C₁-C₆ alkyl optionally substituted with hydroxyl and is different from the other R⁷ that is meta to the CR⁴R⁵ group. In some embodiments of the compound of formula Va, Va-i, Va-ii, Va-iii, and Va-iv, one R⁷ that is meta to the CR⁴R⁵ group is unsubstituted C₁-C₆, alkyl and is different from the other R⁷ that is meta to the CR⁴R⁵ group.

In some embodiments of the compound of formula Va, Va-i, and Va-ii, each R⁶ is C₁-C₆ alkoxy. In some embodiments of the compound of formula Va, Va-i, and Va-ii, each R⁶ is halo. In some embodiments of the compound of formula Va, Va-i, and Va-ii, each R⁶ is C₁-C₆ haloalkyl. In some embodiments of the compound of formula Va, Va-i, and Va-ii, each R⁶ is CN. In some embodiments of the compound of formula Va, Va-i, and Va-ii, each R⁶ is C₃-C₇ cycloalkyl. In some embodiments of the compound of formula Va, Va-i, and Va-ii, each R⁶ is C₁-C₆ alkyl optionally substituted with hydroxyl (e.g., 2-hydroxy-2-propyl). In some embodiments of the compound of formula Va, Va-i, and Va-ii, each R⁶ is unsubstituted C₁-C₆ alkyl. In some embodiments of the compound of formula Va, Va-i, and Va-ii, one R⁶ is hydrogen and is different from the other R⁶. In some embodiments of the compound of formula Va, Va-i, and Va-ii, one R⁶ is C₁-C₆ alkoxy and is different from the other R⁶. In some embodiments of the compound of formula Va, Va-i, and Va-ii, one R⁶ is halo and is different from the other R⁶. In some embodiments of the compound of formula Va, Va-i, and Va-ii, one R⁶ is C₁-C₆ haloalkyl and is different from the other R⁶. In some embodiments of the compound of formula Va, Va-i, and Va-ii, one R⁶ is CN and is different from the other R⁶. In some embodiments of the compound of formula Va, Va-i, and Va-ii, one R⁶ is C₃-C₇ cycloalkyl and is different from the other R⁶. In some embodiments of the compound of formula Va, Va-i, and Va-ii, one R⁶ is C₁-C₆ alkyl optionally substituted with hydroxyl and is different from the other R⁶. In some embodiments of the compound of formula Va, Va-i, and Va-ii, one R⁶ is unsubstituted C₁-C₆ alkyl and is different from the other R⁶.

In some embodiments of the compound of formula Va, Va-i, and Va-ii, at least one pair of R⁶ and R⁷ in adjacent positions, taken together with the carbon atoms connecting them, form a four-membered to seven-membered carbocyclic or heterocyclic ring containing 1 or 2 heteroatoms independently selected from O, N, and S;

wherein the four-membered to seven-membered carbocyclic or heterocyclic ring is optionally independently substituted with one or more substituents selected from F, C₁-C₆ alkyl, C₁-C₆ alkoxy, NR⁸R⁹, oxo, and ═NR¹⁰.

In some more embodiments of the compound of formula Va, Va-i, and Va-ii, the optionally independently substituted four-membered to seven-membered carbocyclic or heterocyclic ring is an optionally independently substituted five-membered carbocyclic ring optionally substituted with one or more F or methyl.

In some more embodiments of the compound of formula Va, Va-i, and Va-ii, the optionally independently substituted four-membered to seven-membered carbocyclic or heterocyclic ring is an optionally independently substituted four-membered carbocyclic ring.

In some embodiments of the compound of formula Va, Va-i, and Va-ii, both pairs of R⁶ and R⁷ in adjacent positions, taken together with the carbon atoms connecting them, each form a four-membered to seven-membered carbocyclic or heterocyclic ring containing 1 or 2 heteroatoms independently selected from O, N, and S;

wherein each four-membered to seven-membered carbocyclic or heterocyclic ring is optionally independently substituted with one or more substituents selected from F, C₁-C₆ alkyl, C₁-C₆ alkoxy, NR⁸R⁹, oxo, and ═NR¹⁰.

In some more embodiments of the compound of formula Va, Va-i, and Va-ii, each optionally independently substituted four-membered to seven-membered carbocyclic or heterocyclic ring is an optionally independently substituted five-membered carbocyclic ring optionally substituted with one or more F or methyl.

In some more embodiments of the compound of formula Va, Va-i, and Va-ii, one optionally independently substituted four-membered to seven-membered carbocyclic or heterocyclic ring is an optionally independently substituted four-membered carbocyclic ring, and the other optionally independently substituted four-membered to seven-membered carbocyclic or heterocyclic ring is an optionally independently substituted five-membered carbocyclic ring optionally substituted with one or more F or methyl.

In some more embodiments of the compound of formula Va, Va-i, and Va-ii, one optionally independently substituted four-membered to seven-membered carbocyclic or heterocyclic ring is an optionally independently substituted five-membered carbocyclic ring, and the other optionally independently substituted four-membered to seven-membered carbocyclic or heterocyclic ring is an optionally independently substituted five-membered carbocyclic ring optionally substituted with one or more F or methyl.

In some embodiments, the compound of formula AA is a compound of Formula Va

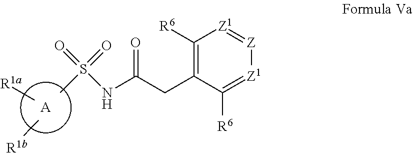

wherein
A is phenyl;
R^{1a} is —SO₂NR¹¹R¹²;
R^{1b} is —OR¹¹;

### Z is N, CH, or CR⁷;

each R⁶ is independently C₁-C₆ alkoxy, halo, C₁-C₆ haloalkyl, C₃-C₇ cycloalkyl, or C₁-C₆ alkyl optionally substituted with hydroxy;
each Z¹ is independently N, CH or CR⁷, each R⁷ is independently C₁-C₆ alkoxy, halo, C₁-C₆ haloalkyl, CN, C₁-C₆ haloalkoxy, C₃-C₇ cycloalkyl, or C₁-C₆ alkyl optionally substituted with hydroxy;
or at least one pair of R⁶ and R⁷ in adjacent positions, taken together with the carbon atoms connecting them, form a four-membered to seven-membered carbocyclic or heterocyclic ring containing 1 or 2 heteroatoms independently selected from O, N, and S; and wherein the four-membered to seven-membered carbocyclic or heterocyclic ring is optionally independently substituted with one or more substituents selected from H, F, C₁-C₆ alkyl, C₁-C₆, alkoxy, NR⁸R⁹, oxo, and ═NR¹⁰.

In some embodiments, the compound of Formula Va is a compound of Formula Va-i:

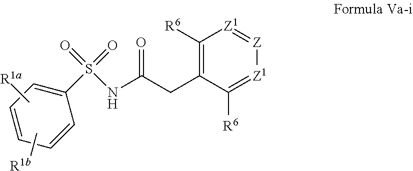

In some embodiments, the compound of Formula Va is a compound of Formula Va-ii:

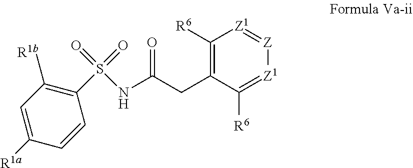

In some embodiments, the compound of Formula Va is a compound of Formula Va-iii:

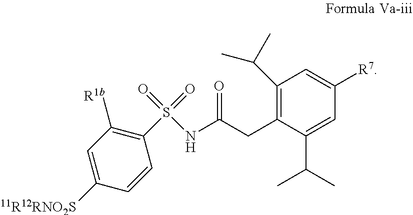

In some embodiments of the compound of formula Va, Va-i, and Va-ii, Z is N. In some embodiments of the compound of formula Va, Va-i, and Va-ii, Z is CR⁷. In some embodiments of the compound of formula Va, Va-i, and Va-ii, Z is CH.

In some embodiments of the compound of formula Va, Va-i, and Va-ii, R^{1a} is methanesulfonamido. In some embodiments of the compound of formula Va, Va-i, and Va-ii, R^{1a} is hydroxymethyl. In some embodiments of the compound of formula Va, Va-i, and Va-ii, R^{1a} is hydroxyethyl. In some embodiments of the compound of formula Va, Va-i, and Va-ii, R^{1a} is 3-hydroxy-1-propyl. In some embodiments of the compound of formula Va, Va-i, and Va-ii, R^{1a} is 2-hydroxy-2-propyl. In some embodiments of the compound of formula Va, Va-i, and Va-ii, R^{1a} is 3-hydroxy-2-propyl. In some embodiments of the compound of formula Va, Va-i, and Va-ii, R^{1a} is 1-hydroxy-1-propyl. In some embodiments of the compound of formula Va, Va-i, and Va-ii, R^{1a} is 2-hydroxy-1-propyl. In some embodiments of the compound of formula Va, Va-i, and Va-ii, R^{1a} is hydroxybutyl. In some embodiments of the compound of formula Va, Va-i, and Va-ii, R^{1a} is hydroxypentyl. In some embodiments of the compound of formula Va, Va-i, and Va-ii, R^{1a} is hydroxyhexyl. In some embodiments of the compound of formula Va, Va-i, and Va-ii, R^{1a} is an unbranched C₁-C₆ alkyl substituted with one hydroxy. In some embodiments of the compound of formula Va, Va-i, and Va-ii, R^{1a} is a branched C₁-C₆ alkyl substituted with one hydroxy.

In some embodiments of the compound of formula Va, Va-i, Va-ii, and Va-iii, R^{1b} is methoxy. In some embodiments of the compound of formula Va, Va-i, Va-ii, and Va-iii, R^{1b} is hydroxymethyl. In some embodiments of the compound of formula Va, Va-i, Va-ii, and Va-iii, R^{1b} is hydroxyethyl. In some embodiments of the compound of formula Va, Va-i, Va-ii, and Va-iii, R^{1b} is 2-hydroxy-2-propyl. In some embodiments of the compound of formula Va, Va-i, Va-ii, and Va-iii, R^{1b} is 3-hydroxy-2-propyl. In some embodiments of the compound of formula Va, Va-i, Va-ii, and Va-iii, R^{1b} is 1-hydroxy-1-propyl. In some embodiments of the compound of formula Va, Va-i, Va-ii, and Va-iii, R^{1b} is 2-hydroxy-1-propyl. In some embodiments of the compound of formula Va, Va-i, Va-ii, and Va-iii, R^{1b} is hydroxybutyl. In some embodiments of the compound of formula Va, Va-i, Va-ii, and Va-iii, R^{1b} is hydroxypentyl. In some embodiments of the compound of formula Va, Va-i, Va-ii, and Va-iii, R^{1b} is hydroxyhexyl. In some embodiments of the compound of formula Va, Va-i, Va-ii, and Va-iii, R^{1b} is an unbranched C₁-C₆ alkyl substituted with one hydroxy. In some embodiments of the compound of formula Va, Va-i, Va-ii, and Va-iii, R^{1b} is a branched C₁-C₆ alkyl substituted with one hydroxy.

In some embodiments of the compound of formula Va, Va-i, Va-ii, Va-iii, and Va-iv, each Z¹ is CH. In some embodiments of the compound of formula Va, Va-i, Va-ii, Va-iii, and Va-iv, one Z¹ is CH and the other Z¹ is CR⁷. In some embodiments of the compound of formula Va, Va-i, and Va-ii, each Z¹ is CR⁷. In some embodiments of the compound of formula Va, Va-i, and Va-ii, Z is CR⁷ wherein R⁷ is CN. In some embodiments of the compound of formula Va, Va-i, and Va-ii, Z is CR⁷ wherein R⁷ is halo (e.g., F). In some embodiments of the compound of formula Va, Va-i, and Va-ii, Z is CR⁷ wherein R⁷ is CO₂C₁-C₆ alkyl. In some embodiments of the compound of formula Va, Va-i, and Va-ii, Z is CR⁷ wherein R⁷ is CONR¹¹R¹²; In some embodiments of the compound of formula Va, Va-i, and Va-ii, Z is CR⁷ wherein R⁷ is C₁-C₆ alkyl. In some embodiments of the compound of formula Va, Va-i, and Va-ii, Z is CR⁷ wherein R⁷ is C₁-C₆ alkoxy; In some embodiments of the compound of formula Va, Va-i, and Va-ii, Z is CR⁷ wherein R⁷ is C₁-C₆, haloalkyl. In some embodiments of the compound of formula Va, Va-i, and Va-ii, each R⁷ that is meta to the CR⁴R⁵ group is C₁-C₆ alkoxy. In some embodiments of the compound of formula Va, Va-i, and Va-ii, each R⁷ that is meta to the CR⁴R⁵ group is halo. In some embodiments of the compound of formula Va, Va-i, and Va-ii, each R⁷ that is meta to the CR⁴R⁵ group is fluoro. In some embodiments of the compound of formula Va, Va-i, and Va-ii, each R⁷ that is meta to the CR⁴R⁵ group is C₁-C₆ haloalkyl. In some embodiments of the compound of formula Va, Va-i, and Va-ii, each R⁷ that is meta to the CR⁴R⁵ group is CN. In some embodiments of the compound of formula Va, Va-i, and Va-ii, each R⁷ that is meta to the CR⁴R⁵ group is C₃-C₇ cycloalkyl. In some embodiments of the compound of formula Va, Va-i, and Va-ii, each R⁷ that is meta to the CR⁴R⁵ group is C i-C₆ alkyl optionally substituted with hydroxyl. In some embodiments of the compound of formula Va, Va-i, and Va-ii, each R⁷ that is meta to the CR⁴R⁵ group is unsubstituted C₁-C₆, alkyl. In some embodiments of the compound of formula Va, Va-i, and Va-ii, one R⁷ that is meta to the CR⁴R⁵ group is hydrogen and is different from the other R⁷ that is meta to the CR⁴R⁵ group. In some embodiments of the compound of formula Va, Va-i, and Va-ii, one R⁷ that is meta to the CR⁴R⁵ group is C₁-C₆ alkoxy and is different from the other R⁷ that is meta to the CR⁴R⁵ group. In some embodiments of the compound of formula Va, Va-i, and Va-ii, one R⁷ that is meta to the CR⁴R⁵ group is halo and is different from the other R⁷ that is meta to the CR⁴R⁵ group. In some embodiments of the compound of formula Va, Va-i, and Va-ii, one R⁷ that is meta to the CR⁴R⁵ group is C₁-C₆ haloalkyl and is different from the other R⁷ that is meta to the CR⁴R⁵ group. In some embodiments of the compound of formula Va, Va-i, and Va-ii, one R⁷ that is meta to the CR⁴R⁵ group is CN and is different from the other R⁷ that is meta to the CR⁴R⁵ group. In some embodiments of the compound of formula Va, Va-i, and Va-ii, one R⁷ that is meta to the CR⁴R⁵ group is C₃-C₇ cycloalkyl and is different from the other R⁷ that is meta to the CR⁴R⁵ group. In some embodiments of the compound of formula Va, Va-i, and Va-ii, one R⁷ that is meta to the CR⁴R⁵ group is C₁-C₆ alkyl optionally substituted with hydroxyl and is different from the other R⁷ that is meta to the CR⁴R⁵ group. In some embodiments of the compound of formula Va, Va-i, and Va-ii, one R⁷ that is meta to the CR⁴R⁵ group is unsubstituted C₁-C₆ alkyl and is different from the other R⁷ that is meta to the CR⁴R⁵ group.

In some embodiments of the compound of formula Va, Va-i, and Va-ii, each R⁶ is C₁-C₆ alkoxy. In some embodiments of the compound of formula Va, Va-i, and Va-ii, each R⁶ is halo. In some embodiments of the compound of formula Va, Va-i, and Va-ii, each R⁶ is C₁-C₆ haloalkyl. In some embodiments of the compound of formula Va, Va-i, and Va-ii, each R⁶ is CN. In some embodiments of the compound of formula Va, Va-i, and Va-ii, each R⁶ is C₃-C₇ cycloalkyl. In some embodiments of the compound of formula Va, Va-i, and Va-ii, each R⁶ is C₁-C₆ alkyl optionally substituted with hydroxyl (e.g., 2-hydroxy-2-propyl). In some embodiments of the compound of formula Va, Va-i, and Va-ii, each R⁶ is unsubstituted C₁-C₆ alkyl. In some embodiments of the compound of formula Va, Va-i, and Va-ii, one R⁶ is C₁-C₆ alkoxy and is different from the other R⁶. In some embodiments of the compound of formula Va, Va-i, and Va-ii, one R⁶ is halo and is different from the other R⁶. In some embodiments of the compound of formula Va, Va-i, and Va-ii, one R⁶ is C₁-C₆ haloalkyl and is different from the other R⁶. In some embodiments of the compound of formula Va, Va-i, and Va-ii, one R⁶ is CN and is different from the other R⁶. In some embodiments of the compound of formula Va, Va-i, and Va-ii, one R⁶ is C₃-C₇ cycloalkyl and is different from the other R⁶. In some embodiments of the compound of formula Va, Va-i, and Va-ii, one R⁶ is C₁-C₆ alkyl optionally substituted with hydroxyl and is different from the other R⁶. In some embodiments of the compound of formula Va, Va-i, and Va-ii, one R⁶ is unsubstituted C₁-C₆ alkyl and is different from the other R⁶.

In some embodiments of the compound of formula Va, Va-i, and Va-ii, at least one pair of R⁶ and R⁷ in adjacent positions, taken together with the carbon atoms connecting them, form a four-membered to seven-membered carbocyclic or heterocyclic ring containing 1 or 2 heteroatoms independently selected from O, N, and S;

wherein the four-membered to seven-membered carbocyclic or heterocyclic ring is optionally independently substituted with one or more substituents selected from F, C₁-C₆ alkyl, C₁-C₆ alkoxy, NR⁸R⁹, oxo, and ═NR¹⁰.

In some more embodiments of the compound of formula Va, Va-i, and Va-ii, the optionally independently substituted four-membered to seven-membered carbocyclic or heterocyclic ring is an optionally independently substituted five-membered carbocyclic ring optionally substituted with one or more F or methyl.

In some more embodiments of the compound of formula Va, Va-i, and Va-ii, the optionally independently substituted four-membered to seven-membered carbocyclic or heterocyclic ring is an optionally independently substituted four-membered carbocyclic ring.

In some embodiments of the compound of formula Va, Va-i, and Va-ii, both pairs of R⁶ and R⁷ in adjacent positions, taken together with the carbon atoms connecting them, each form a four-membered to seven-membered carbocyclic or heterocyclic ring containing 1 or 2 heteroatoms independently selected from O, N, and S;

wherein each four-membered to seven-membered carbocyclic or heterocyclic ring is optionally independently substituted with one or more substituents selected from F, C₁-C₆ alkyl, C₁-C₆ alkoxy, NR⁸R⁹, oxo, and ═NR¹⁰.

In some more embodiments of the compound of formula Va, Va-i, and Va-ii, each optionally independently substituted four-membered to seven-membered carbocyclic or heterocyclic ring is an optionally independently substituted five-membered carbocyclic ring optionally substituted with one or more F or methyl.

In some more embodiments of the compound of formula Va, Va-i, and Va-ii, one optionally independently substituted four-membered to seven-membered carbocyclic or heterocyclic ring is an optionally independently substituted four-membered carbocyclic ring, and the other optionally independently substituted four-membered to seven-membered carbocyclic or heterocyclic ring is an optionally independently substituted five-membered carbocyclic ring optionally substituted with one or more F or methyl.

In some more embodiments of the compound of formula Va, Va-i, and Va-ii, one optionally independently substituted four-membered to seven-membered carbocyclic or heterocyclic ring is an optionally independently substituted five-membered carbocyclic ring, and the other optionally independently substituted four-membered to seven-membered carbocyclic or heterocyclic ring is an optionally independently substituted five-membered carbocyclic ring optionally substituted with one or more F or methyl.

### Non-Limiting Combinations of Substituted Ring a and Substituted Ring B

In some embodiments, the compound of formula AA is a compound wherein the substituted ring

### A is

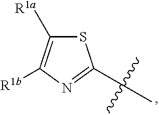

the optionally optionally substituted ring B is

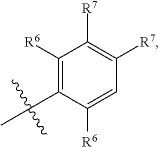

and wherein:
R^{1a} is hydroxymethyl, and R^{1b} is hydroxymethyl; R^{1a} is hydroxymethyl, and R^{1b} is hydroxy ethyl; R^{1a} is hydroxymethyl, and R^{1b} is 2-hydroxy-2-propyl; R^{1a} is hydroxymethyl, and R^{1b} is 3-hydroxy-2-propyl; R^{1a} is hydroxymethyl, and R^{1b} is 1-hydroxy-1-propyl; R^{1a} is hydroxymethyl, and R^{1b} is 2-hydroxy-1-propyl; R^{1a} is hydroxymethyl, and R^{1b} is 3-hydroxy-1-propyl; R^{1a} is hydroxymethyl, and R^{1b} is hydroxybutyl; R^{1a} is hydroxymethyl, and R^{1b} is hydroxypentyl; R^{1a} is hydroxymethyl, and R^{1b} is hydroxyhexyl; R^{1a} is hydroxy ethyl, and R^{1b} is hydroxymethyl; R^{1a} is hydroxy ethyl, and R^{1b} is hydroxy ethyl; R^{1a} is hydroxy ethyl, and R^{1b} is 2-hydroxy-2-propyl; R^{1a} is hydroxy ethyl, and R^{1b} is 3-hydroxy-2-propyl; R^{1a} is hydroxyethyl, and R^{1b} is 1-hydroxy-1-propyl; R^{1a} is hydroxyethyl, and R^{1b} is 2-hydroxy-1-propyl; R^{1a} is hydroxyethyl, and R^{1b} is 3-hydroxy-1-propyl; R^{1a} is hydroxyethyl, and R^{1b} is hydroxybutyl; R^{1a} is hydroxyethyl, and R^{1b} is hydroxypentyl; R^{1a} is hydroxyethyl, and R^{1b} is hydroxyhexyl; R^{1a} is 2-hydroxy-2-propyl, and R^{1b} is hydroxymethyl; R^{1a} is 2-hydroxy-2-propyl, and R^{1b} is hydroxyethyl; R^{1a} is 2-hydroxy-2-propyl, and R^{1b} is 2-hydroxy-2-propyl; R^{1a} is 2-hydroxy-2-propyl, and R^{1b} is 3-hydroxy-2-propyl; R^{1a} is 2-hydroxy-2-propyl, and R^{1b} is 1-hydroxy-1-propyl; R^{1a} is 2-hydroxy-2-propyl, and R^{1b} is 2-hydroxy-1-propyl; R^{1a} is 2-hydroxy-2-propyl, and R^{1b} is 3-hydroxy-1-propyl; R^{1a} is 2-hydroxy-2-propyl, and R^{1b} is hydroxybutyl; R^{1a} is 2-hydroxy-2-propyl, and R^{1b} is hydroxypentyl; R^{1a} is 2-hydroxy-2-propyl, and R^{1b} is hydroxyhexyl; R^{1b} is hydroxymethyl, and R^{1a} is hydroxymethyl; R^{1b} is hydroxymethyl, and R^{1a} is hydroxyethyl; R^{1b} is hydroxymethyl, and R^{1a} is 2-hydroxy-2-propyl; R^{1b} is hydroxymethyl, and R^{1a} is 3-hydroxy-2-propyl; R^{1b} is hydroxymethyl, and R^{1a} is 1-hydroxy-1-propyl; R^{1b} is hydroxymethyl, and R^{1a} is 2-hydroxy-1-propyl; R^{1b} is hydroxymethyl, and R^{1a} is 3-hydroxy-1-propyl; R^{1b} is hydroxymethyl, and R^{1a} is hydroxybutyl; R^{1b} is hydroxymethyl, and R^{1a} is hydroxypentyl; R^{1b} is hydroxymethyl, and R^{1a} is hydroxyhexyl; R^{1b} is hydroxyethyl, and R^{1a} is hydroxymethyl; R^{1b} is hydroxyethyl, and R^{1a} is hydroxyethyl; R^{1b} is hydroxyethyl, and R^{1a} is 2-hydroxy-2-propyl; R^{1b} is hydroxyethyl, and R^{1a} is 3-hydroxy-2-propyl; R^{1b} is hydroxyethyl, and R^{1a} is 1-hydroxy-1-propyl; R^{1b} is hydroxyethyl, and R^{1a} is 2-hydroxy-1-propyl; R^{1b} is hydroxyethyl, and R^{1a} is 3-hydroxy-1-propyl; R^{1b} is hydroxyethyl, and R^{1a} is hydroxybutyl; R^{1b} is hydroxyethyl, and R^{1a} is hydroxypentyl; R^{1b} is hydroxyethyl, and R^{1a} is hydroxyhexyl; R^{1b} is 2-hydroxy-2-propyl, and R^{1a} is hydroxymethyl; R^{1b} is 2-hydroxy-2-propyl, and R^{1a} is hydroxyethyl; R^{1b} is 2-hydroxy-2-propyl, and R^{1a} is 2-hydroxy-2-propyl; R^{1b} is 2-hydroxy-2-propyl, and R^{1a} is 3-hydroxy-2-propyl; R^{1b} is 2-hydroxy-2-propyl, and R^{1a} is 1-hydroxy-1-propyl; R^{1b} is 2-hydroxy-2-propyl, and R^{1a} is 2-hydroxy-1-propyl; R^{1b} is 2-hydroxy-2-propyl, and R^{1a} is 3-hydroxy-1-propyl; R^{1b} is 2-hydroxy-2-propyl, and R^{1a} is hydroxybutyl; R^{1b} is 2-hydroxy-2-propyl, and R^{1a} is hydroxypentyl; R^{1b} is 2-hydroxy-2-propyl, and R^{1a} is hydroxyhexyl;
   and R⁶ and R⁷ are one of the following combinations:
each R⁶ is independently C₁-C₆ alkyl, and R⁷ is C₁-C₆ alkyl optionally substituted with one or more halo; each R⁶ is independently C₁-C₆ alkyl and R⁷ is C₁-C₆ alkyl; each R⁶ is independently C₁-C₆ alkyl, and R⁷ is C₁-C₆ alkyl substituted with one or more halo; each R⁶ is independently C₁-C₆ alkyl, and R⁷ is C₃-C₇ cycloalkyl; each R⁶ is independently C₁-C₆ alkyl, and R⁷ is halo; each R⁶ is independently C₁-C₆ alkyl, and R⁷ is cyano; each R⁶ is independently C₃-C₇ cycloalkyl, and R⁷ is C₃-C₇ cycloalkyl; each R⁶ is independently C₃-C₇ cycloalkyl, and R⁷ is halo; each R⁶ is independently cyclopropyl and R⁷ is halo; each R⁶ is independently C₁-C₆ alkyl, and R⁷ is C₁-C₆ alkoxy optionally substituted with one or more halo; each R⁶ is independently C₁-C₆ alkyl, and R⁷ is C₁-C₆ alkoxy; each R⁶ is independently C₁-C₆ alkyl, and R⁷ is C₁-C₆ alkoxy substituted with one or more halo; each R⁶ is independently halo, and R⁷ is C₁-C₆ haloalkyl; each R⁶ is independently halo, and R⁷ is C₁-C₆ haloalkoxy; each R⁶ is independently C₁-C₆ alkoxy; and R⁷ is halo; each R⁶ is independently C₁-C₆ alkoxy; and R⁷ is chloro; R⁷ is C₁-C₆ alkyl, and each R⁶ is independently C₁-C₆ alkyl optionally substituted with one or more halo; R⁷ is C₁-C₆ alkyl, and each R⁶ is independently C₁-C₆ alkyl substituted with one or more halo; R⁷ is C₁-C₆ alkyl, and each R⁶ is independently C₃-C₇ cycloalkyl; R⁷ is C₁-C₆ alkyl, and each R⁶ is independently halo; R⁷ is C₁-C₆ alkyl and each R⁶ is independently halo; R⁷ is C₁-C₆ alkyl, and R⁶ is cyano; R⁷ is C₃-C₇ cycloalkyl, and each R⁶ is independently C₃-C₇ cycloalkyl; R⁷ is C₃-C₇ cycloalkyl, and each R⁶ is independently halo; R⁷ is C₃-C₇ cycloalkyl and each R⁶ is independently halo; R⁷ is C₁-C₆ alkyl, and each R⁶ is independently C₁-C₆ alkoxy optionally substituted with one or more halo; R⁷ is C₁-C₆ alkyl, and each R⁶ is independently C₁-C₆ alkoxy; R⁷ is C₁-C₆ alkyl, and each R⁶ is independently C₁-C₆ alkoxy substituted with one or more halo; R⁷ is halo, and each R⁶ is independently C₁-C₆ haloalkyl; R⁷ is halo, and each R⁶ is independently C₁-C₆ haloalkoxy; R⁷ is C₁-C₆ alkoxy; and each R⁶ is independently halo; R⁷ is C₁-C₆ alkoxy; and R⁶ is chloro; R⁶ and R⁷ on adjacent atoms taken together with the atoms connecting them form a C₅ aliphatic carbocyclic ring.

In some embodiments, the compound of formula AA is a compound wherein the substituted ring

### A is

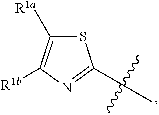

the optionally optionally substituted ring B is

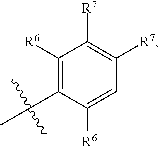

and wherein:
R^{1a} is hydroxymethyl, and R^{1b} is hydroxymethyl; R^{1a} is hydroxymethyl, and R^{1b} is hydroxy ethyl; R^{1a} is hydroxymethyl, and R^{1b} is 2-hydroxy-2-propyl; R^{1a} is hydroxymethyl, and R^{1b} is 3-hydroxy-2-propyl; R^{1a} is hydroxymethyl, and R^{1b} is 1-hydroxy-1-propyl; R^{1a} is hydroxymethyl, and R^{1b} is 2-hydroxy-1-propyl; R^{1a} is hydroxymethyl, and R^{1b} is 3-hydroxy-1-propyl; R^{1a} is hydroxymethyl, and R^{1b} is hydroxybutyl; R^{1a} is hydroxymethyl, and R^{1b} is hydroxypentyl; R^{1a} is hydroxymethyl, and R^{1b} is hydroxyhexyl; R^{1a} is hydroxy ethyl, and R^{1b} is hydroxymethyl; R^{1a} is hydroxy ethyl, and R^{1b} is hydroxy ethyl; R^{1a} is hydroxy ethyl, and R^{1b} is 2-hydroxy-2-propyl; R^{1a} is hydroxy ethyl, and R^{1b} is 3-hydroxy-2-propyl; R^{1a} is hydroxyethyl, and R^{1b} is 1-hydroxy-1-propyl; R^{1a} is hydroxyethyl, and R^{1b} is 2-hydroxy-1-propyl; R^{1a} is hydroxyethyl, and R^{1b} is 3-hydroxy-1-propyl; R^{1a} is hydroxyethyl, and R^{1b} is hydroxybutyl; R^{1a} is hydroxyethyl, and R^{1b} is hydroxypentyl; R^{1a} is hydroxyethyl, and R^{1b} is hydroxyhexyl; R^{1a} is 2-hydroxy-2-propyl, and R^{1b} is hydroxymethyl; R^{1a} is 2-hydroxy-2-propyl, and R^{1b} is hydroxyethyl; R^{1a} is 2-hydroxy-2-propyl, and R^{1b} is 2-hydroxy-2-propyl; R^{1a} is 2-hydroxy-2-propyl, and R^{1b} is 3-hydroxy-2-propyl; R^{1a} is 2-hydroxy-2-propyl, and R^{1b} is 1-hydroxy-1-propyl; R^{1a} is 2-hydroxy-2-propyl, and R^{1b} is 2-hydroxy-1-propyl; R^{1a} is 2-hydroxy-2-propyl, and R^{1b} is 3-hydroxy-1-propyl; R^{1a} is 2-hydroxy-2-propyl, and R^{1b} is hydroxybutyl; R^{1a} is 2-hydroxy-2-propyl, and R^{1b} is hydroxypentyl; R^{1a} is 2-hydroxy-2-propyl, and R^{1b} is hydroxyhexyl; R^{1b} is hydroxymethyl, and R^{1a} is hydroxymethyl; R^{1b} is hydroxymethyl, and R^{1a} is hydroxyethyl; R^{1b} is hydroxymethyl, and R^{1a} is 2-hydroxy-2-propyl; R^{1b} is hydroxymethyl, and R^{1a} is 3-hydroxy-2-propyl; R^{1b} is hydroxymethyl, and R^{1a} is 1-hydroxy-1-propyl; R^{1b} is hydroxymethyl, and R^{1a} is 2-hydroxy-1-propyl; R^{1b} is hydroxymethyl, and R^{1a} is 3-hydroxy-1-propyl; R^{1b} is hydroxymethyl, and R^{1a} is hydroxybutyl; R^{1b} is hydroxymethyl, and R^{1a} is hydroxypentyl; R^{1b} is hydroxymethyl, and R^{1a} is hydroxyhexyl; R^{1b} is hydroxyethyl, and R^{1a} is hydroxymethyl; R^{1b} is hydroxyethyl, and R^{1a} is hydroxyethyl; R^{1b} is hydroxyethyl, and R^{1a} is 2-hydroxy-2-propyl; R^{1b} is hydroxyethyl, and R^{1a} is 3-hydroxy-2-propyl; R^{1b} is hydroxyethyl, and R^{1a} is 1-hydroxy-1-propyl; R^{1b} is hydroxyethyl, and R^{1a} is 2-hydroxy-1-propyl; R^{1b} is hydroxyethyl, and R^{1a} is 3-hydroxy-1-propyl; R^{1b} is hydroxyethyl, and R^{1a} is hydroxybutyl; R^{1b} is hydroxyethyl, and R^{1a} is hydroxypentyl; R^{1b} is hydroxyethyl, and R^{1a} is hydroxyhexyl; R^{1b} is 2-hydroxy-2-propyl, and R^{1a} is hydroxymethyl; R^{1b} is 2-hydroxy-2-propyl, and R^{1a} is hydroxyethyl; R^{1b} is 2-hydroxy-2-propyl, and R^{1a} is 2-hydroxy-2-propyl; R^{1b} is 2-hydroxy-2-propyl, and R^{1a} is 3-hydroxy-2-propyl; R^{1b} is 2-hydroxy-2-propyl, and R^{1a} is 1-hydroxy-1-propyl; R^{1b} is 2-hydroxy-2-propyl, and R^{1a} is 2-hydroxy-1-propyl; R^{1b} is 2-hydroxy-2-propyl, and R^{1a} is 3-hydroxy-1-propyl; R^{1b} is 2-hydroxy-2-propyl, and R^{1a} is hydroxybutyl; R^{1b} is 2-hydroxy-2-propyl, and R^{1a} is hydroxypentyl; R^{1b} is 2-hydroxy-2-propyl, and R^{1a} is hydroxyhexyl;
   and R⁶ and R⁷ are one of the following combinations:
each R⁶ is isopropyl; and R⁷ is methyl; each R⁶ is isopropyl; and R⁷ is isopropyl; each R⁶ is isopropyl; and R⁷ is trifluoromethyl; each R⁶ is isopropyl; and R⁷ is cyclopropyl; each R⁶ is isopropyl; and R⁷ is chloro; each R⁶ is isopropyl; and R⁷ is fluoro; each R⁶ is ethyl; and R⁷ is fluoro; each R⁶ is isopropyl; and R⁷ is cyano; each R⁶ is cyclopropyl; and R⁷ is cyclopropyl; each R⁶ is cyclopropyl; and R⁷ is chloro; each R⁶ is cyclopropyl; and R⁷ is fluoro; each R⁶ is isopropyl; and R⁷ is methoxy; each R⁶ is isopropyl; and R⁷ is trifluoromethoxy; each R⁶ is chloro; and R⁷ is trifluoromethyl; each R⁶ is chloro; and R⁷ is trifluoromethoxy; R⁷ is isopropyl; and each R⁶ is methyl; R⁷ is isopropyl; and each R⁶ is trifluoromethyl; R⁷ is isopropyl; and each R⁶ is cyclopropyl; R⁷ is isopropyl; and each R⁶ is chloro; R⁷ is ethyl; and each R⁶ is fluoro; R⁷ is isopropyl; and each R⁶ is cyano; R⁷ is cyclopropyl; and each R⁶ is cyclopropyl; R⁷ is cyclopropyl; and each R⁶ is chloro; R⁷ is cyclopropyl; and each R⁶ is fluoro; R⁷ is isopropyl; and each R⁶ is methoxy; R⁷ is isopropyl; and each R⁶ is trifluoromethoxy; R⁷ is chloro; and each R⁶ is trifluoromethyl; R⁷ is chloro; and each R⁶ is trifluoromethoxy; one R⁶ is isopropyl; the other R⁶ is trifluoromethyl; and R⁷ is chloro; R⁶ and R⁷ on adjacent atoms taken together with the atoms connecting them form a C₅ aliphatic carbocyclic ring; and one R⁶ is fluoro, chloro, or cyano.

In some embodiments, the compound of formula AA is a compound wherein the substituted ring

### A is

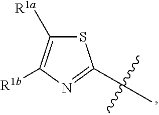

the optionally optionally substituted ring B is

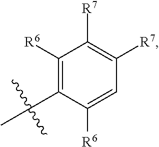

and wherein:
R^{1a} is hydroxymethyl, and R^{1b} is hydroxymethyl; R^{1a} is hydroxymethyl, and R^{1b} is hydroxy ethyl; R^{1a} is hydroxymethyl, and R^{1b} is 2-hydroxy-2-propyl; R^{1a} is hydroxymethyl, and R^{1b} is 3-hydroxy-2-propyl; R^{1a} is hydroxymethyl, and R^{1b} is 1-hydroxy-1-propyl; R^{1a} is hydroxymethyl, and R^{1b} is 2-hydroxy-1-propyl; R^{1a} is hydroxymethyl, and R^{1b} is 3-hydroxy-1-propyl; R^{1a} is hydroxymethyl, and R^{1b} is hydroxybutyl; R^{1a} is hydroxymethyl, and R^{1b} is hydroxypentyl; R^{1a} is hydroxymethyl, and R^{1b} is hydroxyhexyl; R^{1a} is hydroxy ethyl, and R^{1b} is hydroxymethyl; R^{1a} is hydroxy ethyl, and R^{1b} is hydroxy ethyl; R^{1a} is hydroxy ethyl, and R^{1b} is 2-hydroxy-2-propyl; R^{1a} is hydroxy ethyl, and R^{1b} is 3-hydroxy-2-propyl; R^{1a} is hydroxyethyl, and R^{1b} is 1-hydroxy-1-propyl; R^{1a} is hydroxyethyl, and R^{1b} is 2-hydroxy-1-propyl; R^{1a} is hydroxyethyl, and R^{1b} is 3-hydroxy-1-propyl; R^{1a} is hydroxyethyl, and R^{1b} is hydroxybutyl; R^{1a} is hydroxyethyl, and R^{1b} is hydroxypentyl; R^{1a} is hydroxyethyl, and R^{1b} is hydroxyhexyl; R^{1a} is 2-hydroxy-2-propyl, and R^{1b} is hydroxymethyl; R^{1a} is 2-hydroxy-2-propyl, and R^{1b} is hydroxyethyl; R^{1a} is 2-hydroxy-2-propyl, and R^{1b} is 2-hydroxy-2-propyl; R^{1a} is 2-hydroxy-2-propyl, and R^{1b} is 3-hydroxy-2-propyl; R^{1a} is 2-hydroxy-2-propyl, and R^{1b} is 1-hydroxy-1-propyl; R^{1a} is 2-hydroxy-2-propyl, and R^{1b} is 2-hydroxy-1-propyl; R^{1a} is 2-hydroxy-2-propyl, and R^{1b} is 3-hydroxy-1-propyl; R^{1a} is 2-hydroxy-2-propyl, and R^{1b} is hydroxybutyl; R^{1a} is 2-hydroxy-2-propyl, and R^{1b} is hydroxypentyl; R^{1a} is 2-hydroxy-2-propyl, and R^{1b} is hydroxyhexyl; R^{1b} is hydroxymethyl, and R^{1a} is hydroxymethyl; R^{1b} is hydroxymethyl, and R^{1a} is hydroxyethyl; R^{1b} is hydroxymethyl, and R^{1a} is 2-hydroxy-2-propyl; R^{1b} is hydroxymethyl, and R^{1a} is 3-hydroxy-2-propyl; R^{1b} is hydroxymethyl, and R^{1a} is 1-hydroxy-1-propyl; R^{1b} is hydroxymethyl, and R^{1a} is 2-hydroxy-1-propyl; R^{1b} is hydroxymethyl, and R^{1a} is 3-hydroxy-1-propyl; R^{1b} is hydroxymethyl, and R^{1a} is hydroxybutyl; R^{1b} is hydroxymethyl, and R^{1a} is hydroxypentyl; R^{1b} is hydroxymethyl, and R^{1a} is hydroxyhexyl; R^{1b} is hydroxyethyl, and R^{1a} is hydroxymethyl; R^{1b} is hydroxyethyl, and R^{1a} is hydroxyethyl; R^{1b} is hydroxyethyl, and R^{1a} is 2-hydroxy-2-propyl; R^{1b} is hydroxyethyl, and R^{1a} is 3-hydroxy-2-propyl; R^{1b} is hydroxyethyl, and R^{1a} is 1-hydroxy-1-propyl; R^{1b} is hydroxyethyl, and R^{1a} is 2-hydroxy-1-propyl; R^{1b} is hydroxyethyl, and R^{1a} is 3-hydroxy-1-propyl; R^{1b} is hydroxyethyl, and R^{1a} is hydroxybutyl; R^{1b} is hydroxyethyl, and R^{1a} is hydroxypentyl; R^{1b} is hydroxyethyl, and R^{1a} is hydroxyhexyl; R^{1b} is 2-hydroxy-2-propyl, and R^{1a} is hydroxymethyl; R^{1b} is 2-hydroxy-2-propyl, and R^{1a} is hydroxyethyl; R^{1b} is 2-hydroxy-2-propyl, and R^{1a} is 2-hydroxy-2-propyl; R^{1b} is 2-hydroxy-2-propyl, and R^{1a} is 3-hydroxy-2-propyl; R^{1b} is 2-hydroxy-2-propyl, and R^{1a} is 1-hydroxy-1-propyl; R^{1b} is 2-hydroxy-2-propyl, and R^{1a} is 2-hydroxy-1-propyl; R^{1b} is 2-hydroxy-2-propyl, and R^{1a} is 3-hydroxy-1-propyl; R^{1b} is 2-hydroxy-2-propyl, and R^{1a} is hydroxybutyl; R^{1b} is 2-hydroxy-2-propyl, and R^{1a} is hydroxypentyl; R^{1b} is 2-hydroxy-2-propyl, and R^{1a} is hydroxyhexyl;
   and R⁶ and R⁷ are one of the following combinations:
each R⁶ is independently C₁-C₆ alkyl, and R⁷ is C₁-C₆ alkyl optionally substituted with one or more halo; each R⁶ is independently C₁-C₆ alkyl and R⁷ is C₁-C₆ alkyl; each R⁶ is independently C₁-C₆ alkyl, and R⁷ is C₁-C₆ alkyl substituted with one or more halo; each R⁶ is independently C₁-C₆ alkyl, and R⁷ is C₃-C₇ cycloalkyl; each R⁶ is independently C₁-C₆ alkyl, and R⁷ is halo; each R⁶ is independently C₁-C₆ alkyl, and R⁷ is cyano; each R⁶ is independently C₃-C₇ cycloalkyl, and R⁷ is C₃-C₇ cycloalkyl; each R⁶ is independently C₃-C₇ cycloalkyl, and R⁷ is halo; each R⁶ is independently cyclopropyl and R⁷ is halo; each R⁶ is independently C₁-C₆ alkyl, and R⁷ is C₁-C₆ alkoxy optionally substituted with one or more halo; each R⁶ is independently C₁-C₆ alkyl, and R⁷ is C₁-C₆ alkoxy; each R⁶ is independently C₁-C₆ alkyl, and R⁷ is C₁-C₆ alkoxy substituted with one or more halo; each R⁶ is independently halo, and R⁷ is C₁-C₆ haloalkyl; each R⁶ is independently halo, and R⁷ is C₁-C₆ haloalkoxy; each R⁶ is independently C₁-C₆ alkoxy; and R⁷ is halo; each R⁶ is independently C₁-C₆ alkoxy; and R⁷ is chloro; R⁷ is C₁-C₆ alkyl, and each R⁶ is independently C₁-C₆ alkyl optionally substituted with one or more halo; R⁷ is C₁-C₆ alkyl, and each R⁶ is independently C₁-C₆ alkyl substituted with one or more halo; R⁷ is C₁-C₆ alkyl, and each R⁶ is independently C₃-C₇ cycloalkyl; R⁷ is C₁-C₆ alkyl, and each R⁶ is independently halo; R⁷ is C₁-C₆ alkyl and each R⁶ is independently halo; R⁷ is C₁-C₆ alkyl, and R⁶ is cyano; R⁷ is C₃-C₇ cycloalkyl, and each R⁶ is independently C₃-C₇ cycloalkyl; R⁷ is C₃-C₇ cycloalkyl, and each R⁶ is independently halo; R⁷ is C₃-C₇ cycloalkyl and each R⁶ is independently halo; R⁷ is C₁-C₆ alkyl, and each R⁶ is independently C₁-C₆, alkoxy optionally substituted with one or more halo; R⁷ is C₁-C₆ alkyl, and each R⁶ is independently C₁-C₆ alkoxy; R⁷ is C₁-C₆ alkyl, and each R⁶ is independently C₁-C₆ alkoxy substituted with one or more halo; R⁷ is halo, and each R⁶ is independently C₁-C₆ haloalkyl; R⁷ is halo, and each R⁶ is independently C₁-C₆ haloalkoxy; R⁷ is C₁-C₆ alkoxy; and each R⁶ is independently halo; R⁷ is C₁-C₆ alkoxy; and R⁶ is chloro; R⁶ and R⁷ on adjacent atoms taken together with the atoms connecting them form a C₅ aliphatic carbocyclic ring.

In some embodiments, the compound of formula AA is a compound wherein the substituted ring

### A is

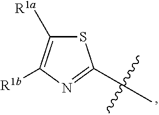

the optionally optionally substituted ring B is

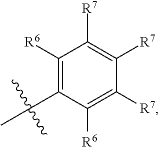

and wherein:
R^{1a} is hydroxymethyl, and R^{1b} is hydroxymethyl; R^{1a} is hydroxymethyl, and R^{1b} is hydroxy ethyl; R^{1a} is hydroxymethyl, and R^{1b} is 2-hydroxy-2-propyl; R^{1a} is hydroxymethyl, and R^{1b} is 3-hydroxy-2-propyl; R^{1a} is hydroxymethyl, and R^{1b} is 1-hydroxy-1-propyl; R^{1a} is hydroxymethyl, and R^{1b} is 2-hydroxy-1-propyl; R^{1a} is hydroxymethyl, and R^{1b} is 3-hydroxy-1-propyl; R^{1a} is hydroxymethyl, and R^{1b} is hydroxybutyl; R^{1a} is hydroxymethyl, and R^{1b} is hydroxypentyl; R^{1a} is hydroxymethyl, and R^{1b} is hydroxyhexyl; R^{1a} is hydroxy ethyl, and R^{1b} is hydroxymethyl; R^{1a} is hydroxy ethyl, and R^{1b} is hydroxy ethyl; R^{1a} is hydroxy ethyl, and R^{1b} is 2-hydroxy-2-propyl; R^{1a} is hydroxy ethyl, and R^{1b} is 3-hydroxy-2-propyl; R^{1a} is hydroxyethyl, and R^{1b} is 1-hydroxy-1-propyl; R^{1a} is hydroxyethyl, and R^{1b} is 2-hydroxy-1-propyl; R^{1a} is hydroxyethyl, and R^{1b} is 3-hydroxy-1-propyl; R^{1a} is hydroxyethyl, and R^{1b} is hydroxybutyl; R^{1a} is hydroxyethyl, and R^{1b} is hydroxypentyl; R^{1a} is hydroxyethyl, and R^{1b} is hydroxyhexyl; R^{1a} is 2-hydroxy-2-propyl, and R^{1b} is hydroxymethyl; R^{1a} is 2-hydroxy-2-propyl, and R^{1b} is hydroxyethyl; R^{1a} is 2-hydroxy-2-propyl, and R^{1b} is 2-hydroxy-2-propyl; R^{1a} is 2-hydroxy-2-propyl, and R^{1b} is 3-hydroxy-2-propyl; R^{1a} is 2-hydroxy-2-propyl, and R^{1b} is 1-hydroxy-1-propyl; R^{1a} is 2-hydroxy-2-propyl, and R^{1b} is 2-hydroxy-1-propyl; R^{1a} is 2-hydroxy-2-propyl, and R^{1b} is 3-hydroxy-1-propyl; R^{1a} is 2-hydroxy-2-propyl, and R^{1b} is hydroxybutyl; R^{1a} is 2-hydroxy-2-propyl, and R^{1b} is hydroxypentyl; R^{1a} is 2-hydroxy-2-propyl, and R^{1b} is hydroxyhexyl; R^{1b} is hydroxymethyl, and R^{1a} is hydroxymethyl; R^{1b} is hydroxymethyl, and R^{1a} is hydroxyethyl; R^{1b} is hydroxymethyl, and R^{1a} is 2-hydroxy-2-propyl; R^{1b} is hydroxymethyl, and R^{1a} is 3-hydroxy-2-propyl; R^{1b} is hydroxymethyl, and R^{1a} is 1-hydroxy-1-propyl; R^{1b} is hydroxymethyl, and R^{1a} is 2-hydroxy-1-propyl; R^{1b} is hydroxymethyl, and R^{1a} is 3-hydroxy-1-propyl; R^{1b} is hydroxymethyl, and R^{1a} is hydroxybutyl; R^{1b} is hydroxymethyl, and R^{1a} is hydroxypentyl; R^{1b} is hydroxymethyl, and R^{1a} is hydroxyhexyl; R^{1b} is hydroxyethyl, and R^{1a} is hydroxymethyl; R^{1b} is hydroxyethyl, and R^{1a} is hydroxyethyl; R^{1b} is hydroxyethyl, and R^{1a} is 2-hydroxy-2-propyl; R^{1b} is hydroxyethyl, and R^{1a} is 3-hydroxy-2-propyl; R^{1b} is hydroxyethyl, and R^{1a} is 1-hydroxy-1-propyl; R^{1b} is hydroxy ethyl, and R^{1a} is 2-hydroxy-1-propyl; R^{1b} is hydroxy ethyl, and R^{1a} is 3-hydroxy-1-propyl; R^{1b} is hydroxy ethyl, and R^{1a} is hydroxybutyl; R^{1b} is hydroxy ethyl, and R^{1a} is hydroxypentyl; R^{1b} is hydroxyethyl, and R^{1a} is hydroxyhexyl; R^{1b} is 2-hydroxy-2-propyl, and R^{1a} is hydroxymethyl; R^{1b} is 2-hydroxy-2-propyl, and R^{1a} is hydroxyethyl; R^{1b} is 2-hydroxy-2-propyl, and R^{1a} is 2-hydroxy-2-propyl; R^{1b} is 2-hydroxy-2-propyl, and R^{1a} is 3-hydroxy-2-propyl; R^{1b} is 2-hydroxy-2-propyl, and R^{1a} is 1-hydroxy-1-propyl; R^{1b} is 2-hydroxy-2-propyl, and R^{1a} is 2-hydroxy-1-propyl; R^{1b} is 2-hydroxy-2-propyl, and R^{1a} is 3-hydroxy-1-propyl; R^{1b} is 2-hydroxy-2-propyl, and R^{1a} is hydroxybutyl; R^{1b} is 2-hydroxy-2-propyl, and R^{1a} is hydroxypentyl; R^{1b} is 2-hydroxy-2-propyl, and R^{1a} is hydroxyhexyl;
   and R⁶ and R⁷ are one of the following combinations:
each R⁶ is isopropyl; and R⁷ is methyl; each R⁶ is isopropyl; and R⁷ is isopropyl; each R⁶ is isopropyl; and R⁷ is trifluoromethyl; each R⁶ is isopropyl; and R⁷ is cyclopropyl; each R⁶ is isopropyl; and R⁷ is chloro; each R⁶ is isopropyl; and R⁷ is fluoro; each R⁶ is ethyl; and R⁷ is fluoro; each R⁶ is isopropyl; and R⁷ is cyano; each R⁶ is cyclopropyl; and R⁷ is cyclopropyl; each R⁶ is cyclopropyl; and R⁷ is chloro; each R⁶ is cyclopropyl; and R⁷ is fluoro; each R⁶ is isopropyl; and R⁷ is methoxy; each R⁶ is isopropyl; and R⁷ is trifluoromethoxy; each R⁶ is chloro; and R⁷ is trifluoromethyl; each R⁶ is chloro; and R⁷ is trifluoromethoxy; R⁷ is isopropyl; and each R⁶ is methyl; R⁷ is isopropyl; and each R⁶ is trifluoromethyl; R⁷ is isopropyl; and each R⁶ is cyclopropyl; R⁷ is isopropyl; and each R⁶ is chloro; R⁷ is ethyl; and each R⁶ is fluoro; R⁷ is isopropyl; and each R⁶ is cyano; R⁷ is cyclopropyl; and each R⁶ is cyclopropyl; R⁷ is cyclopropyl; and each R⁶ is chloro; R⁷ is cyclopropyl; and each R⁶ is fluoro; R⁷ is isopropyl; and each R⁶ is methoxy; R⁷ is isopropyl; and each R⁶ is trifluoromethoxy; R⁷ is chloro; and each R⁶ is trifluoromethyl; R⁷ is chloro; and each R⁶ is trifluoromethoxy; one R⁶ is isopropyl; the other R⁶ is trifluoromethyl; and R⁷ is chloro; R⁶ and R⁷ on adjacent atoms taken together with the atoms connecting them form a C₅ aliphatic carbocyclic ring; and one R⁶ is fluoro, chloro, or cyano.

In some embodiments, the compound of formula AA is a compound wherein the substituted ring

### A is

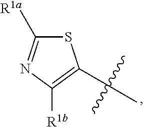

the optionally optionally substituted ring B is

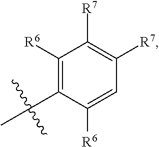

R⁶, and wherein:
R^{1a} is hydroxymethyl, and R^{1b} is hydroxymethyl; R^{1a} is hydroxymethyl, and R^{1b} is hydroxy ethyl; R^{1a} is hydroxymethyl, and R^{1b} is 2-hydroxy-2-propyl; R^{1a} is hydroxymethyl, and R^{1b} is 3-hydroxy-2-propyl; R^{1a} is hydroxymethyl, and R^{1b} is 1-hydroxy-1-propyl; R^{1a} is hydroxymethyl, and R^{1b} is 2-hydroxy-1-propyl; R^{1a} is hydroxymethyl, and R^{1b} is 3-hydroxy-1-propyl; R^{1a} is hydroxymethyl, and R^{1b} is hydroxybutyl; R^{1a} is hydroxymethyl, and R^{1b} is hydroxypentyl; R^{1a} is hydroxymethyl, and R^{1b} is hydroxyhexyl; R^{1a} is hydroxy ethyl, and R^{1b} is hydroxymethyl; R^{1a} is hydroxy ethyl, and R^{1b} is hydroxy ethyl; R^{1a} is hydroxy ethyl, and R^{1b} is 2-hydroxy-2-propyl; R^{1a} is hydroxy ethyl, and R^{1b} is 3-hydroxy-2-propyl; R^{1a} is hydroxyethyl, and R^{1b} is 1-hydroxy-1-propyl; R^{1a} is hydroxyethyl, and R^{1b} is 2-hydroxy-1-propyl; R^{1a} is hydroxyethyl, and R^{1b} is 3-hydroxy-1-propyl; R^{1a} is hydroxyethyl, and R^{1b} is hydroxybutyl; R^{1a} is hydroxyethyl, and R^{1b} is hydroxypentyl; R^{1a} is hydroxyethyl, and R^{1b} is hydroxyhexyl; R^{1a} is 2-hydroxy-2-propyl, and R^{1b} is hydroxymethyl; R^{1a} is 2-hydroxy-2-propyl, and R^{1b} is hydroxyethyl; R^{1a} is 2-hydroxy-2-propyl, and R^{1b} is 2-hydroxy-2-propyl; R^{1a} is 2-hydroxy-2-propyl, and R^{1b} is 3-hydroxy-2-propyl; R^{1a} is 2-hydroxy-2-propyl, and R^{1b} is 1-hydroxy-1-propyl; R^{1a} is 2-hydroxy-2-propyl, and R^{1b} is 2-hydroxy-1-propyl; R^{1a} is 2-hydroxy-2-propyl, and R^{1b} is 3-hydroxy-1-propyl; R^{1a} is 2-hydroxy-2-propyl, and R^{1b} is hydroxybutyl; R^{1a} is 2-hydroxy-2-propyl, and R^{1b} is hydroxypentyl; R^{1a} is 2-hydroxy-2-propyl, and R^{1b} is hydroxyhexyl; R^{1b} is hydroxymethyl, and R^{1a} is hydroxymethyl; R^{1b} is hydroxymethyl, and R^{1a} is hydroxyethyl; R^{1b} is hydroxymethyl, and R^{1a} is 2-hydroxy-2-propyl; R^{1b} is hydroxymethyl, and R^{1a} is 3-hydroxy-2-propyl; R^{1b} is hydroxymethyl, and R^{1a} is 1-hydroxy-1-propyl; R^{1b} is hydroxymethyl, and R^{1a} is 2-hydroxy-1-propyl; R^{1b} is hydroxymethyl, and R^{1a} is 3-hydroxy-1-propyl; R^{1b} is hydroxymethyl, and R^{1a} is hydroxybutyl; R^{1b} is hydroxymethyl, and R^{1a} is hydroxypentyl; R^{1b} is hydroxymethyl, and R^{1a} is hydroxyhexyl; R^{1b} is hydroxyethyl, and R^{1a} is hydroxymethyl; R^{1b} is hydroxyethyl, and R^{1a} is hydroxyethyl; R^{1b} is hydroxyethyl, and R^{1a} is 2-hydroxy-2-propyl; R^{1b} is hydroxyethyl, and R^{1a} is 3-hydroxy-2-propyl; R^{1b} is hydroxyethyl, and R^{1a} is 1-hydroxy-1-propyl; R^{1b} is hydroxy ethyl, and R^{1a} is 2-hydroxy-1-propyl; R^{1b} is hydroxy ethyl, and R^{1a} is 3-hydroxy-1-propyl; R^{1b} is hydroxy ethyl, and R^{1a} is hydroxybutyl; R^{1b} is hydroxy ethyl, and R^{1a} is hydroxypentyl; R^{1b} is hydroxyethyl, and R^{1a} is hydroxyhexyl; R^{1b} is 2-hydroxy-2-propyl, and R^{1a} is hydroxymethyl; R^{1b} is 2-hydroxy-2-propyl, and R^{1a} is hydroxyethyl; R^{1b} is 2-hydroxy-2-propyl, and R^{1a} is 2-hydroxy-2-propyl; R^{1b} is 2-hydroxy-2-propyl, and R^{1a} is 3-hydroxy-2-propyl; R^{1b} is 2-hydroxy-2-propyl, and R^{1a} is 1-hydroxy-1-propyl; R^{1b} is 2-hydroxy-2-propyl, and R^{1a} is 2-hydroxy-1-propyl; R^{1b} is 2-hydroxy-2-propyl, and R^{1a} is 3-hydroxy-1-propyl; R^{1b} is 2-hydroxy-2-propyl, and R^{1a} is hydroxybutyl; R^{1b} is 2-hydroxy-2-propyl, and R^{1a} is hydroxypentyl; R^{1b} is 2-hydroxy-2-propyl, and R^{1a} is hydroxyhexyl;
   and R⁶ and R⁷ are one of the following combinations:
each R⁶ is independently C₁-C₆ alkyl, and R⁷ is C₁-C₆ alkyl optionally substituted with one or more halo; each R⁶ is independently C₁-C₆ alkyl and R⁷ is C₁-C₆ alkyl; each R⁶ is independently C₁-C₆ alkyl, and R⁷ is C₁-C₆ alkyl substituted with one or more halo; each R⁶ is independently C₁-C₆ alkyl, and R⁷ is C₃-C₇ cycloalkyl; each R⁶ is independently C₁-C₆ alkyl, and R⁷ is halo; each R⁶ is independently C₁-C₆ alkyl, and R⁷ is cyano; each R⁶ is independently C₃-C₇ cycloalkyl, and R⁷ is C₃-C₇ cycloalkyl; each R⁶ is independently C₃-C₇ cycloalkyl, and R⁷ is halo; each R⁶ is independently cyclopropyl and R⁷ is halo; each R⁶ is independently C₁-C₆ alkyl, and R⁷ is C₁-C₆ alkoxy optionally substituted with one or more halo; each R⁶ is independently C₁-C₆ alkyl, and R⁷ is C₁-C₆ alkoxy; each R⁶ is independently C₁-C₆ alkyl, and R⁷ is C₁-C₆ alkoxy substituted with one or more halo; each R⁶ is independently halo, and R⁷ is C₁-C₆ haloalkyl; each R⁶ is independently halo, and R⁷ is C₁-C₆ haloalkoxy; each R⁶ is independently C₁-C₆ alkoxy; and R⁷ is halo; each R⁶ is independently C₁-C₆ alkoxy; and R⁷ is chloro; R⁷ is C₁-C₆ alkyl, and each R⁶ is independently C₁-C₆ alkyl optionally substituted with one or more halo; R⁷ is C₁-C₆ alkyl, and each R⁶ is independently C₁-C₆ alkyl substituted with one or more halo; R⁷ is C₁-C₆ alkyl, and each R⁶ is independently C₃-C₇ cycloalkyl; R⁷ is C₁-C₆ alkyl, and each R⁶ is independently halo; R⁷ is C₁-C₆ alkyl and each R⁶ is independently halo; R⁷ is C₁-C₆ alkyl, and R⁶ is cyano; R⁷ is C₃-C₇ cycloalkyl, and each R⁶ is independently C₃-C₇ cycloalkyl; R⁷ is C₃-C₇ cycloalkyl, and each R⁶ is independently halo; R⁷ is C₃-C₇ cycloalkyl and each R⁶ is independently halo; R⁷ is C₁-C₆ alkyl, and each R⁶ is independently C₁-C₆, alkoxy optionally substituted with one or more halo; R⁷ is C₁-C₆ alkyl, and each R⁶ is independently C₁-C₆ alkoxy; R⁷ is C₁-C₆ alkyl, and each R⁶ is independently C₁-C₆ alkoxy substituted with one or more halo; R⁷ is halo, and each R⁶ is independently C₁-C₆ haloalkyl; R⁷ is halo, and each R⁶ is independently C₁-C₆ haloalkoxy; R⁷ is C₁-C₆ alkoxy; and each R⁶ is independently halo; R⁷ is C₁-C₆ alkoxy; and R⁶ is chloro; R⁶ and R⁷ on adjacent atoms taken together with the atoms connecting them form a C₅ aliphatic carbocyclic ring.

In some embodiments, the compound of formula AA is a compound wherein the substituted ring

### A is

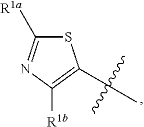

the optionally optionally substituted ring B is

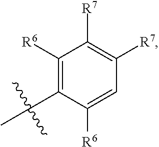

and wherein:
R^{1a} is hydroxymethyl, and R^{1b} is hydroxymethyl; R^{1a} is hydroxymethyl, and R^{1b} is hydroxy ethyl; R^{1a} is hydroxymethyl, and R^{1b} is 2-hydroxy-2-propyl; R^{1a} is hydroxymethyl, and R^{1b} is 3-hydroxy-2-propyl; R^{1a} is hydroxymethyl, and R^{1b} is 1-hydroxy-1-propyl; R^{1a} is hydroxymethyl, and R^{1b} is 2-hydroxy-1-propyl; R^{1a} is hydroxymethyl, and R^{1b} is 3-hydroxy-1-propyl; R^{1a} is hydroxymethyl, and R^{1b} is hydroxybutyl; R^{1a} is hydroxymethyl, and R^{1b} is hydroxypentyl; R^{1a} is hydroxymethyl, and R^{1b} is hydroxyhexyl; R^{1a} is hydroxy ethyl, and R^{1b} is hydroxymethyl; R^{1a} is hydroxy ethyl, and R^{1b} is hydroxy ethyl; R^{1a} is hydroxy ethyl, and R^{1b} is 2-hydroxy-2-propyl; R^{1a} is hydroxy ethyl, and R^{1b} is 3-hydroxy-2-propyl; R^{1a} is hydroxyethyl, and R^{1b} is 1-hydroxy-1-propyl; R^{1a} is hydroxyethyl, and R^{1b} is 2-hydroxy-1-propyl; R^{1a} is hydroxyethyl, and R^{1b} is 3-hydroxy-1-propyl; R^{1a} is hydroxyethyl, and R^{1b} is hydroxybutyl; R^{1a} is hydroxyethyl, and R^{1b} is hydroxypentyl; R^{1a} is hydroxyethyl, and R^{1b} is hydroxyhexyl; R^{1a} is 2-hydroxy-2-propyl, and R^{1b} is hydroxymethyl; R^{1a} is 2-hydroxy-2-propyl, and R^{1b} is hydroxyethyl; R^{1a} is 2-hydroxy-2-propyl, and R^{1b} is 2-hydroxy-2-propyl; R^{1a} is 2-hydroxy-2-propyl, and R^{1b} is 3-hydroxy-2-propyl; R^{1a} is 2-hydroxy-2-propyl, and R^{1b} is 1-hydroxy-1-propyl; R^{1a} is 2-hydroxy-2-propyl, and R^{1b} is 2-hydroxy-1-propyl; R^{1a} is 2-hydroxy-2-propyl, and R^{1b} is 3-hydroxy-1-propyl; R^{1a} is 2-hydroxy-2-propyl, and R^{1b} is hydroxybutyl; R^{1a} is 2-hydroxy-2-propyl, and R^{1b} is hydroxypentyl; R^{1a} is 2-hydroxy-2-propyl, and R^{1b} is hydroxyhexyl; R^{1b} is hydroxymethyl, and R^{1a} is hydroxymethyl; R^{1b} is hydroxymethyl, and R^{1a} is hydroxyethyl; R^{1b} is hydroxymethyl, and R^{1a} is 2-hydroxy-2-propyl; R^{1b} is hydroxymethyl, and R^{1a} is 3-hydroxy-2-propyl; R^{1b} is hydroxymethyl, and R^{1a} is 1-hydroxy-1-propyl; R^{1b} is hydroxymethyl, and R^{1a} is 2-hydroxy-1-propyl; R^{1b} is hydroxymethyl, and R^{1a} is 3-hydroxy-1-propyl; R^{1b} is hydroxymethyl, and R^{1a} is hydroxybutyl; R^{1b} is hydroxymethyl, and R^{1a} is hydroxypentyl; R^{1b} is hydroxymethyl, and R^{1a} is hydroxyhexyl; R^{1b} is hydroxy ethyl, and R^{1a} is hydroxymethyl; R^{1b} is hydroxy ethyl, and R^{1a} is hydroxyethyl; R^{1b} is hydroxyethyl, and R^{1a} is 2-hydroxy-2-propyl; R^{1b} is hydroxyethyl, and R^{1a} is 3-hydroxy-2-propyl; R^{1b} is hydroxyethyl, and R^{1a} is 1-hydroxy-1-propyl; R^{1b} is hydroxyethyl, and R^{1a} is 2-hydroxy-1-propyl; R^{1b} is hydroxyethyl, and R^{1a} is 3-hydroxy-1-propyl; R^{1b} is hydroxyethyl, and R^{1a} is hydroxybutyl; R^{1b} is hydroxyethyl, and R^{1a} is hydroxypentyl; R^{1b} is hydroxyethyl, and R^{1a} is hydroxyhexyl; R^{1b} is 2-hydroxy-2-propyl, and R^{1a} is hydroxymethyl; R^{1b} is 2-hydroxy-2-propyl, and R^{1a} is hydroxyethyl; R^{1b} is 2-hydroxy-2-propyl, and R^{1a} is 2-hydroxy-2-propyl; R^{1b} is 2-hydroxy-2-propyl, and R^{1a} is 3-hydroxy-2-propyl; R^{1b} is 2-hydroxy-2-propyl, and R^{1a} is 1-hydroxy-1-propyl; R^{1b} is 2-hydroxy-2-propyl, and R^{1a} is 2-hydroxy-1-propyl; R^{1b} is 2-hydroxy-2-propyl, and R^{1a} is 3-hydroxy-1-propyl; R^{1b} is 2-hydroxy-2-propyl, and R^{1a} is hydroxybutyl; R^{1b} is 2-hydroxy-2-propyl, and R^{1a} is hydroxypentyl; R^{1b} is 2-hydroxy-2-propyl, and R^{1a} is hydroxyhexyl;
   and R⁶ and R⁷ are one of the following combinations:
each R⁶ is isopropyl; and R⁷ is methyl; each R⁶ is isopropyl; and R⁷ is isopropyl; each R⁶ is isopropyl; and R⁷ is trifluoromethyl; each R⁶ is isopropyl; and R⁷ is cyclopropyl; each R⁶ is isopropyl; and R⁷ is chloro; each R⁶ is isopropyl; and R⁷ is fluoro; each R⁶ is ethyl; and R⁷ is fluoro; each R⁶ is isopropyl; and R⁷ is cyano; each R⁶ is cyclopropyl; and R⁷ is cyclopropyl; each R⁶ is cyclopropyl; and R⁷ is chloro; each R⁶ is cyclopropyl; and R⁷ is fluoro; each R⁶ is isopropyl; and R⁷ is methoxy; each R⁶ is isopropyl; and R⁷ is trifluoromethoxy; each R⁶ is chloro; and R⁷ is trifluoromethyl; each R⁶ is chloro; and R⁷ is trifluoromethoxy; R⁷ is isopropyl; and each R⁶ is methyl; R⁷ is isopropyl; and each R⁶ is trifluoromethyl; R⁷ is isopropyl; and each R⁶ is cyclopropyl; R⁷ is isopropyl; and each R⁶ is chloro; R⁷ is ethyl; and each R⁶ is fluoro; R⁷ is isopropyl; and each R⁶ is cyano; R⁷ is cyclopropyl; and each R⁶ is cyclopropyl; R⁷ is cyclopropyl; and each R⁶ is chloro; R⁷ is cyclopropyl; and each R⁶ is fluoro; R⁷ is isopropyl; and each R⁶ is methoxy; R⁷ is isopropyl; and each R⁶ is trifluoromethoxy; R⁷ is chloro; and each R⁶ is trifluoromethyl; R⁷ is chloro; and each R⁶ is trifluoromethoxy; one R⁶ is isopropyl; the other R⁶ is trifluoromethyl; and R⁷ is chloro; R⁶ and R⁷ on adjacent atoms taken together with the atoms connecting them form a C₅ aliphatic carbocyclic ring; and one R⁶ is fluoro, chloro, or cyano.

In some embodiments, the compound of formula AA is a compound wherein the substituted ring

### A is

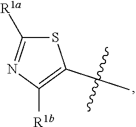

the optionally optionally substituted ring B is

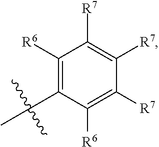

R⁶, and wherein:
R^{1a} is hydroxymethyl, and R^{1b} is hydroxymethyl; R^{1a} is hydroxymethyl, and R^{1b} is hydroxy ethyl; R^{1a} is hydroxymethyl, and R^{1b} is 2-hydroxy-2-propyl; R^{1a} is hydroxymethyl, and R^{1b} is 3-hydroxy-2-propyl; R^{1a} is hydroxymethyl, and R^{1b} is 1-hydroxy-1-propyl; R^{1a} is hydroxymethyl, and R^{1b} is 2-hydroxy-1-propyl; R^{1a} is hydroxymethyl, and R^{1b} is 3-hydroxy-1-propyl; R^{1a} is hydroxymethyl, and R^{1b} is hydroxybutyl; R^{1a} is hydroxymethyl, and R^{1b} is hydroxypentyl; R^{1a} is hydroxymethyl, and R^{1b} is hydroxyhexyl; R^{1a} is hydroxy ethyl, and R^{1b} is hydroxymethyl; R^{1a} is hydroxy ethyl, and R^{1b} is hydroxy ethyl; R^{1a} is hydroxy ethyl, and R^{1b} is 2-hydroxy-2-propyl; R^{1a} is hydroxy ethyl, and R^{1b} is 3-hydroxy-2-propyl; R^{1a} is hydroxyethyl, and R^{1b} is 1-hydroxy-1-propyl; R^{1a} is hydroxyethyl, and R^{1b} is 2-hydroxy-1-propyl; R^{1a} is hydroxyethyl, and R^{1b} is 3-hydroxy-1-propyl; R^{1a} is hydroxyethyl, and R^{1b} is hydroxybutyl; R^{1a} is hydroxyethyl, and R^{1b} is hydroxypentyl; R^{1a} is hydroxyethyl, and R^{1b} is hydroxyhexyl; R^{1a} is 2-hydroxy-2-propyl, and R^{1b} is hydroxymethyl; R^{1a} is 2-hydroxy-2-propyl, and R^{1b} is hydroxyethyl; R^{1a} is 2-hydroxy-2-propyl, and R^{1b} is 2-hydroxy-2-propyl; R^{1a} is 2-hydroxy-2-propyl, and R^{1b} is 3-hydroxy-2-propyl; R^{1a} is 2-hydroxy-2-propyl, and R^{1b} is 1-hydroxy-1-propyl; R^{1a} is 2-hydroxy-2-propyl, and R^{1b} is 2-hydroxy-1-propyl; R^{1a} is 2-hydroxy-2-propyl, and R^{1b} is 3-hydroxy-1-propyl; R^{1a} is 2-hydroxy-2-propyl, and R^{1b} is hydroxybutyl; R^{1a} is 2-hydroxy-2-propyl, and R^{1b} is hydroxypentyl; R^{1a} is 2-hydroxy-2-propyl, and R^{1b} is hydroxyhexyl; R^{1b} is hydroxymethyl, and R^{1a} is hydroxymethyl; R^{1b} is hydroxymethyl, and R^{1a} is hydroxyethyl; R^{1b} is hydroxymethyl, and R^{1a} is 2-hydroxy-2-propyl; R^{1b} is hydroxymethyl, and R^{1a} is 3-hydroxy-2-propyl; R^{1b} is hydroxymethyl, and R^{1a} is 1-hydroxy-1-propyl; R^{1b} is hydroxymethyl, and R^{1a} is 2-hydroxy-1-propyl; R^{1b} is hydroxymethyl, and R^{1a} is 3-hydroxy-1-propyl; R^{1b} is hydroxymethyl, and R^{1a} is hydroxybutyl; R^{1b} is hydroxymethyl, and R^{1a} is hydroxypentyl; R^{1b} is hydroxymethyl, and R^{1a} is hydroxyhexyl; R^{1b} is hydroxyethyl, and R^{1a} is hydroxymethyl; R^{1b} is hydroxyethyl, and R^{1a} is hydroxyethyl; R^{1b} is hydroxyethyl, and R^{1a} is 2-hydroxy-2-propyl; R^{1b} is hydroxyethyl, and R^{1a} is 3-hydroxy-2-propyl; R^{1b} is hydroxyethyl, and R^{1a} is 1-hydroxy-1-propyl; R^{1b} is hydroxy ethyl, and R^{1a} is 2-hydroxy-1-propyl; R^{1b} is hydroxyethyl, and R^{1a} is 3-hydroxy-1-propyl; R^{1b} is hydroxyethyl, and R^{1a} is hydroxybutyl; R^{1b} is hydroxyethyl, and R^{1a} is hydroxypentyl; R^{1b} is hydroxyethyl, and R^{1a} is hydroxyhexyl; R^{1b} is 2-hydroxy-2-propyl, and R^{1a} is hydroxymethyl; R^{1b} is 2-hydroxy-2-propyl, and R^{1a} is hydroxyethyl; R^{1b} is 2-hydroxy-2-propyl, and R^{1a} is 2-hydroxy-2-propyl; R^{1b} is 2-hydroxy-2-propyl, and R^{1a} is 3-hydroxy-2-propyl; R^{1b} is 2-hydroxy-2-propyl, and R^{1a} is 1-hydroxy-1-propyl; R^{1b} is 2-hydroxy-2-propyl, and R^{1a} is 2-hydroxy-1-propyl; R^{1b} is 2-hydroxy-2-propyl, and R^{1a} is 3-hydroxy-1-propyl; R^{1b} is 2-hydroxy-2-propyl, and R^{1a} is hydroxybutyl; R^{1b} is 2-hydroxy-2-propyl, and R^{1a} is hydroxypentyl; R^{1b} is 2-hydroxy-2-propyl, and R^{1a} is hydroxyhexyl;
   and R⁶ and R⁷ are one of the following combinations:
each R⁶ is independently C₁-C₆ alkyl, and R⁷ is C₁-C₆ alkyl optionally substituted with one or more halo; each R⁶ is independently C₁-C₆ alkyl and R⁷ is C₁-C₆ alkyl; each R⁶ is independently C₁-C₆ alkyl, and R⁷ is C₁-C₆ alkyl substituted with one or more halo; each R⁶ is independently C₁-C₆ alkyl, and R⁷ is C₃-C₇ cycloalkyl; each R⁶ is independently C₁-C₆ alkyl, and R⁷ is halo; each R⁶ is independently C₁-C₆ alkyl, and R⁷ is cyano; each R⁶ is independently C₃-C₇ cycloalkyl, and R⁷ is C₃-C₇ cycloalkyl; each R⁶ is independently C₃-C₇ cycloalkyl, and R⁷ is halo; each R⁶ is independently cyclopropyl and R⁷ is halo; each R⁶ is independently C₁-C₆ alkyl, and R⁷ is C₁-C₆ alkoxy optionally substituted with one or more halo; each R⁶ is independently C₁-C₆ alkyl, and R⁷ is C₁-C₆ alkoxy; each R⁶ is independently C₁-C₆ alkyl, and R⁷ is C₁-C₆ alkoxy substituted with one or more halo; each R⁶ is independently halo, and R⁷ is C₁-C₆ haloalkyl; each R⁶ is independently halo, and R⁷ is C₁-C₆ haloalkoxy; each R⁶ is independently C₁-C₆ alkoxy; and R⁷ is halo; each R⁶ is independently C₁-C₆ alkoxy; and R⁷ is chloro; R⁷ is C₁-C₆ alkyl, and each R⁶ is independently C₁-C₆ alkyl optionally substituted with one or more halo; R⁷ is C₁-C₆ alkyl, and each R⁶ is independently C₁-C₆ alkyl substituted with one or more halo; R⁷ is C₁-C₆ alkyl, and each R⁶ is independently C₃-C₇ cycloalkyl; R⁷ is C₁-C₆ alkyl, and each R⁶ is independently halo; R⁷ is C₁-C₆ alkyl and each R⁶ is independently halo; R⁷ is C₁-C₆ alkyl, and R⁶ is cyano; R⁷ is C₃-C₇ cycloalkyl, and each R⁶ is independently C₃-C₇ cycloalkyl; R⁷ is C₃-C₇ cycloalkyl, and each R⁶ is independently halo; R⁷ is C₃-C₇ cycloalkyl and each R⁶ is independently halo; R⁷ is C₁-C₆ alkyl, and each R⁶ is independently C₁-C₆, alkoxy optionally substituted with one or more halo; R⁷ is C₁-C₆ alkyl, and each R⁶ is independently C₁-C₆ alkoxy; R⁷ is C₁-C₆ alkyl, and each R⁶ is independently C₁-C₆ alkoxy substituted with one or more halo; R⁷ is halo, and each R⁶ is independently C₁-C₆ haloalkyl; R⁷ is halo, and each R⁶ is independently C₁-C₆ haloalkoxy; R⁷ is C₁-C₆ alkoxy; and each R⁶ is independently halo; R⁷ is C₁-C₆ alkoxy; and R⁶ is chloro; R⁶ and R⁷ on adjacent atoms taken together with the atoms connecting them form a C₅ aliphatic carbocyclic ring.

In some embodiments, the compound of formula AA is a compound wherein the substituted ring A is

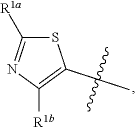

the optionally optionally substituted ring B is

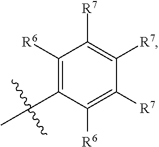

R⁶, and wherein:
R^{1a} is hydroxymethyl, and R^{1b} is hydroxymethyl; R^{1a} is hydroxymethyl, and R^{1b} is hydroxy ethyl; R^{1a} is hydroxymethyl, and R^{1b} is 2-hydroxy-2-propyl; R^{1a} is hydroxymethyl, and R^{1b} is 3-hydroxy-2-propyl; R^{1a} is hydroxymethyl, and R^{1b} is 1-hydroxy-1-propyl; R^{1a} is hydroxymethyl, and R^{1b} is 2-hydroxy-1-propyl; R^{1a} is hydroxymethyl, and R^{1b} is 3-hydroxy-1-propyl; R^{1a} is hydroxymethyl, and R^{1b} is hydroxybutyl; R^{1a} is hydroxymethyl, and R^{1b} is hydroxypentyl; R^{1a} is hydroxymethyl, and R^{1b} is hydroxyhexyl; R^{1a} is hydroxy ethyl, and R^{1b} is hydroxymethyl; R^{1a} is hydroxy ethyl, and R^{1b} is hydroxy ethyl; R^{1a} is hydroxy ethyl, and R^{1b} is 2-hydroxy-2-propyl; R^{1a} is hydroxy ethyl, and R^{1b} is 3-hydroxy-2-propyl; R^{1a} is hydroxyethyl, and R^{1b} is 1-hydroxy-1-propyl; R^{1a} is hydroxyethyl, and R^{1b} is 2-hydroxy-1-propyl; R^{1a} is hydroxyethyl, and R^{1b} is 3-hydroxy-1-propyl; R^{1a} is hydroxyethyl, and R^{1b} is hydroxybutyl; R^{1a} is hydroxyethyl, and R^{1b} is hydroxypentyl; R^{1a} is hydroxyethyl, and R^{1b} is hydroxyhexyl; R^{1a} is 2-hydroxy-2-propyl, and R^{1b} is hydroxymethyl; R^{1a} is 2-hydroxy-2-propyl, and R^{1b} is hydroxyethyl; R^{1a} is 2-hydroxy-2-propyl, and R^{1b} is 2-hydroxy-2-propyl; R^{1a} is 2-hydroxy-2-propyl, and R^{1b} is 3-hydroxy-2-propyl; R^{1a} is 2-hydroxy-2-propyl, and R^{1b} is 1-hydroxy-1-propyl; R^{1a} is 2-hydroxy-2-propyl, and R^{1b} is 2-hydroxy-1-propyl; R^{1a} is 2-hydroxy-2-propyl, and R^{1b} is 3-hydroxy-1-propyl; R^{1a} is 2-hydroxy-2-propyl, and R^{1b} is hydroxybutyl; R^{1a} is 2-hydroxy-2-propyl, and R^{1b} is hydroxypentyl; R^{1a} is 2-hydroxy-2-propyl, and R^{1b} is hydroxyhexyl; R^{1b} is hydroxymethyl, and R^{1a} is hydroxymethyl; R^{1b} is hydroxymethyl, and R^{1a} is hydroxyethyl; R^{1b} is hydroxymethyl, and R^{1a} is 2-hydroxy-2-propyl; R^{1b} is hydroxymethyl, and R^{1a} is 3-hydroxy-2-propyl; R^{1b} is hydroxymethyl, and R^{1a} is 1-hydroxy-1-propyl; R^{1b} is hydroxymethyl, and R^{1a} is 2-hydroxy-1-propyl; R^{1b} is hydroxymethyl, and R^{1a} is 3-hydroxy-1-propyl; R^{1b} is hydroxymethyl, and R^{1a} is hydroxybutyl; R^{1b} is hydroxymethyl, and R^{1a} is hydroxypentyl; R^{1b} is hydroxymethyl, and R^{1a} is hydroxyhexyl; R^{1b} is hydroxy ethyl, and R^{1a} is hydroxymethyl; R^{1b} is hydroxy ethyl, and R^{1a} is hydroxyethyl; R^{1b} is hydroxyethyl, and R^{1a} is 2-hydroxy-2-propyl; R^{1b} is hydroxyethyl, and R^{1a} is 3-hydroxy-2-propyl; R^{1b} is hydroxyethyl, and R^{1a} is 1-hydroxy-1-propyl; R^{1b} is hydroxyethyl, and R^{1a} is 2-hydroxy-1-propyl; R^{1b} is hydroxyethyl, and R^{1a} is 3-hydroxy-1-propyl; R^{1b} is hydroxyethyl, and R^{1a} is hydroxybutyl; R^{1b} is hydroxyethyl, and R^{1a} is hydroxypentyl; R^{1b} is hydroxyethyl, and R^{1a} is hydroxyhexyl; R^{1b} is 2-hydroxy-2-propyl, and R^{1a} is hydroxymethyl; R^{1b} is 2-hydroxy-2-propyl, and R^{1a} is hydroxyethyl; R^{1b} is 2-hydroxy-2-propyl, and R^{1a} is 2-hydroxy-2-propyl; R^{1b} is 2-hydroxy-2-propyl, and R^{1a} is 3-hydroxy-2-propyl; R^{1b} is 2-hydroxy-2-propyl, and R^{1a} is 1-hydroxy-1-propyl; R^{1b} is 2-hydroxy-2-propyl, and R^{1a} is 2-hydroxy-1-propyl; R^{1b} is 2-hydroxy-2-propyl, and R^{1a} is 3-hydroxy-1-propyl; R^{1b} is 2-hydroxy-2-propyl, and R^{1a} is hydroxybutyl; R^{1b} is 2-hydroxy-2-propyl, and R^{1a} is hydroxypentyl; R^{1b} is 2-hydroxy-2-propyl, and R^{1a} is hydroxyhexyl;
   and R⁶ and R⁷ are one of the following combinations:
each R⁶ is isopropyl; and R⁷ is methyl; each R⁶ is isopropyl; and R⁷ is isopropyl; each R⁶ is isopropyl; and R⁷ is trifluoromethyl; each R⁶ is isopropyl; and R⁷ is cyclopropyl; each R⁶ is isopropyl; and R⁷ is chloro; each R⁶ is isopropyl; and R⁷ is fluoro; each R⁶ is ethyl; and R⁷ is fluoro; each R⁶ is isopropyl; and R⁷ is cyano; each R⁶ is cyclopropyl; and R⁷ is cyclopropyl; each R⁶ is cyclopropyl; and R⁷ is chloro; each R⁶ is cyclopropyl; and R⁷ is fluoro; each R⁶ is isopropyl; and R⁷ is methoxy; each R⁶ is isopropyl; and R⁷ is trifluoromethoxy; each R⁶ is chloro; and R⁷ is trifluoromethyl; each R⁶ is chloro; and R⁷ is trifluoromethoxy; R⁷ is isopropyl; and each R⁶ is methyl; R⁷ is isopropyl; and each R⁶ is trifluoromethyl; R⁷ is isopropyl; and each R⁶ is cyclopropyl; R⁷ is isopropyl; and each R⁶ is chloro; R⁷ is ethyl; and each R⁶ is fluoro; R⁷ is isopropyl; and each R⁶ is cyano; R⁷ is cyclopropyl; and each R⁶ is cyclopropyl; R⁷ is cyclopropyl; and each R⁶ is chloro; R⁷ is cyclopropyl; and each R⁶ is fluoro; R⁷ is isopropyl; and each R⁶ is methoxy; R⁷ is isopropyl; and each R⁶ is trifluoromethoxy; R⁷ is chloro; and each R⁶ is trifluoromethyl; R⁷ is chloro; and each R⁶ is trifluoromethoxy; one R⁶ is isopropyl; the other R⁶ is trifluoromethyl; and R⁷ is chloro; R⁶ and R⁷ on adjacent atoms taken together with the atoms connecting them form a C₅ aliphatic carbocyclic ring; and one R⁶ is fluoro, chloro, or cyano.

In some embodiments, the compound of formula AA is a compound wherein the substituted ring

### A is

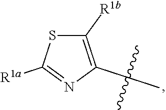

the optionally optionally substituted ring B is

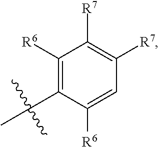

and wherein:
R^{1a} is hydroxymethyl, and R^{1b} is hydroxymethyl; R^{1a} is hydroxymethyl, and R^{1b} is hydroxy ethyl; R^{1a} is hydroxymethyl, and R^{1b} is 2-hydroxy-2-propyl; R^{1a} is hydroxymethyl, and R^{1b} is 3-hydroxy-2-propyl; R^{1a} is hydroxymethyl, and R^{1b} is 1-hydroxy-1-propyl; R^{1a} is hydroxymethyl, and R^{1b} is 2-hydroxy-1-propyl; R^{1a} is hydroxymethyl, and R^{1b} is 3-hydroxy-1-propyl; R^{1a} is hydroxymethyl, and R^{1b} is hydroxybutyl; R^{1a} is hydroxymethyl, and R^{1b} is hydroxypentyl; R^{1a} is hydroxymethyl, and R^{1b} is hydroxyhexyl; R^{1a} is hydroxy ethyl, and R^{1b} is hydroxymethyl; R^{1a} is hydroxy ethyl, and R^{1b} is hydroxy ethyl; R^{1a} is hydroxy ethyl, and R^{1b} is 2-hydroxy-2-propyl; R^{1a} is hydroxy ethyl, and R^{1b} is 3-hydroxy-2-propyl; R^{1a} is hydroxyethyl, and R^{1b} is 1-hydroxy-1-propyl; R^{1a} is hydroxyethyl, and R^{1b} is 2-hydroxy-1-propyl; R^{1a} is hydroxyethyl, and R^{1b} is 3-hydroxy-1-propyl; R^{1a} is hydroxyethyl, and R^{1b} is hydroxybutyl; R^{1a} is hydroxyethyl, and R^{1b} is hydroxypentyl; R^{1a} is hydroxyethyl, and R^{1b} is hydroxyhexyl; R^{1a} is 2-hydroxy-2-propyl, and R^{1b} is hydroxymethyl; R^{1a} is 2-hydroxy-2-propyl, and R^{1b} is hydroxyethyl; R^{1a} is 2-hydroxy-2-propyl, and R^{1b} is 2-hydroxy-2-propyl; R^{1a} is 2-hydroxy-2-propyl, and R^{1b} is 3-hydroxy-2-propyl; R^{1a} is 2-hydroxy-2-propyl, and R^{1b} is 1-hydroxy-1-propyl; R^{1a} is 2-hydroxy-2-propyl, and R^{1b} is 2-hydroxy-1-propyl; R^{1a} is 2-hydroxy-2-propyl, and R^{1b} is 3-hydroxy-1-propyl; R^{1a} is 2-hydroxy-2-propyl, and R^{1b} is hydroxybutyl; R^{1a} is 2-hydroxy-2-propyl, and R^{1b} is hydroxypentyl; R^{1a} is 2-hydroxy-2-propyl, and R^{1b} is hydroxyhexyl; R^{1b} is hydroxymethyl, and R^{1a} is hydroxymethyl; R^{1b} is hydroxymethyl, and R^{1a} is hydroxyethyl; R^{1b} is hydroxymethyl, and R^{1a} is 2-hydroxy-2-propyl; R^{1b} is hydroxymethyl, and R^{1a} is 3-hydroxy-2-propyl; R^{1b} is hydroxymethyl, and R^{1a} is 1-hydroxy-1-propyl; R^{1b} is hydroxymethyl, and R^{1a} is 2-hydroxy-1-propyl; R^{1b} is hydroxymethyl, and R^{1a} is 3-hydroxy-1-propyl; R^{1b} is hydroxymethyl, and R^{1a} is hydroxybutyl; R^{1b} is hydroxymethyl, and R^{1a} is hydroxypentyl; R^{1b} is hydroxymethyl, and R^{1a} is hydroxyhexyl; R^{1b} is hydroxy ethyl, and R^{1a} is hydroxymethyl; R^{1b} is hydroxy ethyl, and R^{1a} is hydroxyethyl; R^{1b} is hydroxyethyl, and R^{1a} is 2-hydroxy-2-propyl; R^{1b} is hydroxyethyl, and R^{1a} is 3-hydroxy-2-propyl; R^{1b} is hydroxyethyl, and R^{1a} is 1-hydroxy-1-propyl; R^{1b} is hydroxyethyl, and R^{1a} is 2-hydroxy-1-propyl; R^{1b} is hydroxyethyl, and R^{1a} is 3-hydroxy-1-propyl; R^{1b} is hydroxyethyl, and R^{1a} is hydroxybutyl; R^{1b} is hydroxyethyl, and R^{1a} is hydroxypentyl; R^{1b} is hydroxyethyl, and R^{1a} is hydroxyhexyl; R^{1b} is 2-hydroxy-2-propyl, and R^{1a} is hydroxymethyl; R^{1b} is 2-hydroxy-2-propyl, and R^{1a} is hydroxyethyl; R^{1b} is 2-hydroxy-2-propyl, and R^{1a} is 2-hydroxy-2-propyl; R^{1b} is 2-hydroxy-2-propyl, and R^{1a} is 3-hydroxy-2-propyl; R^{1b} is 2-hydroxy-2-propyl, and R^{1a} is 1-hydroxy-1-propyl; R^{1b} is 2-hydroxy-2-propyl, and R^{1a} is 2-hydroxy-1-propyl; R^{1b} is 2-hydroxy-2-propyl, and R^{1a} is 3-hydroxy-1-propyl; R^{1b} is 2-hydroxy-2-propyl, and R^{1a} is hydroxybutyl; R^{1b} is 2-hydroxy-2-propyl, and R^{1a} is hydroxypentyl; R^{1b} is 2-hydroxy-2-propyl, and R^{1a} is hydroxyhexyl;
   and R⁶ and R⁷ are one of the following combinations:
each R⁶ is independently C₁-C₆ alkyl, and R⁷ is C₁-C₆ alkyl optionally substituted with one or more halo; each R⁶ is independently C₁-C₆ alkyl and R⁷ is C₁-C₆ alkyl; each R⁶ is independently C₁-C₆ alkyl, and R⁷ is C₁-C₆ alkyl substituted with one or more halo; each R⁶ is independently C₁-C₆ alkyl, and R⁷ is C₃-C₇ cycloalkyl; each R⁶ is independently C₁-C₆ alkyl, and R⁷ is halo; each R⁶ is independently C₁-C₆ alkyl, and R⁷ is cyano; each R⁶ is independently C₃-C₇ cycloalkyl, and R⁷ is C₃-C₇ cycloalkyl; each R⁶ is independently C₃-C₇ cycloalkyl, and R⁷ is halo; each R⁶ is independently cyclopropyl and R⁷ is halo; each R⁶ is independently C₁-C₆ alkyl, and R⁷ is C₁-C₆ alkoxy optionally substituted with one or more halo; each R⁶ is independently C₁-C₆ alkyl, and R⁷ is C₁-C₆ alkoxy; each R⁶ is independently C₁-C₆ alkyl, and R⁷ is C₁-C₆ alkoxy substituted with one or more halo; each R⁶ is independently halo, and R⁷ is C₁-C₆ haloalkyl; each R⁶ is independently halo, and R⁷ is C₁-C₆ haloalkoxy; each R⁶ is independently C₁-C₆ alkoxy; and R⁷ is halo; each R⁶ is independently C₁-C₆ alkoxy; and R⁷ is chloro; R⁷ is C₁-C₆ alkyl, and each R⁶ is independently C₁-C₆ alkyl optionally substituted with one or more halo; R⁷ is C₁-C₆ alkyl, and each R⁶ is independently C₁-C₆ alkyl substituted with one or more halo; R⁷ is C₁-C₆ alkyl, and each R⁶ is independently C₃-C₇ cycloalkyl; R⁷ is C₁-C₆ alkyl, and each R⁶ is independently halo; R⁷ is C₁-C₆ alkyl and each R⁶ is independently halo; R⁷ is C₁-C₆ alkyl, and R⁶ is cyano; R⁷ is C₃-C₇ cycloalkyl, and each R⁶ is independently C₃-C₇ cycloalkyl; R⁷ is C₃-C₇ cycloalkyl, and each R⁶ is independently halo; R⁷ is C₃-C₇ cycloalkyl and each R⁶ is independently halo; R⁷ is C₁-C₆ alkyl, and each R⁶ is independently C₁-C₆, alkoxy optionally substituted with one or more halo; R⁷ is C₁-C₆ alkyl, and each R⁶ is independently C₁-C₆ alkoxy; R⁷ is C₁-C₆ alkyl, and each R⁶ is independently C₁-C₆ alkoxy substituted with one or more halo; R⁷ is halo, and each R⁶ is independently C₁-C₆ haloalkyl; R⁷ is halo, and each R⁶ is independently C₁-C₆ haloalkoxy; R⁷ is C₁-C₆ alkoxy; and each R⁶ is independently halo; R⁷ is C₁-C₆ alkoxy; and R⁶ is chloro; R⁶ and R⁷ on adjacent atoms taken together with the atoms connecting them form a C₅ aliphatic carbocyclic ring.

In some embodiments, the compound of formula AA is a compound wherein the substituted ring

### A is

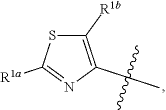

the optionally optionally substituted ring B is

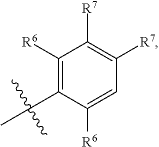

and wherein:
R^{1a} is hydroxymethyl, and R^{1b} is hydroxymethyl; R^{1a} is hydroxymethyl, and R^{1b} is hydroxy ethyl; R^{1a} is hydroxymethyl, and R^{1b} is 2-hydroxy-2-propyl; R^{1a} is hydroxymethyl, and R^{1b} is 3-hydroxy-2-propyl; R^{1a} is hydroxymethyl, and R^{1b} is 1-hydroxy-1-propyl; R^{1a} is hydroxymethyl, and R^{1b} is 2-hydroxy-1-propyl; R^{1a} is hydroxymethyl, and R^{1b} is 3-hydroxy-1-propyl; R^{1a} is hydroxymethyl, and R^{1b} is hydroxybutyl; R^{1a} is hydroxymethyl, and R^{1b} is hydroxypentyl; R^{1a} is hydroxymethyl, and R^{1b} is hydroxyhexyl; R^{1a} is hydroxy ethyl, and R^{1b} is hydroxymethyl; R^{1a} is hydroxy ethyl, and R^{1b} is hydroxy ethyl; R^{1a} is hydroxy ethyl, and R^{1b} is 2-hydroxy-2-propyl; R^{1a} is hydroxy ethyl, and R^{1b} is 3-hydroxy-2-propyl; R^{1a} is hydroxyethyl, and R^{1b} is 1-hydroxy-1-propyl; R^{1a} is hydroxyethyl, and R^{1b} is 2-hydroxy-1-propyl; R^{1a} is hydroxyethyl, and R^{1b} is 3-hydroxy-1-propyl; R^{1a} is hydroxyethyl, and R^{1b} is hydroxybutyl; R^{1a} is hydroxyethyl, and R^{1b} is hydroxypentyl; R^{1a} is hydroxyethyl, and R^{1b} is hydroxyhexyl; R^{1a} is 2-hydroxy-2-propyl, and R^{1b} is hydroxymethyl; R^{1a} is 2-hydroxy-2-propyl, and R^{1b} is hydroxyethyl; R^{1a} is 2-hydroxy-2-propyl, and R^{1b} is 2-hydroxy-2-propyl; R^{1a} is 2-hydroxy-2-propyl, and R^{1b} is 3-hydroxy-2-propyl; R^{1a} is 2-hydroxy-2-propyl, and R^{1b} is 1-hydroxy-1-propyl; R^{1a} is 2-hydroxy-2-propyl, and R^{1b} is 2-hydroxy-1-propyl; R^{1a} is 2-hydroxy-2-propyl, and R^{1b} is 3-hydroxy-1-propyl; R^{1a} is 2-hydroxy-2-propyl, and R^{1b} is hydroxybutyl; R^{1a} is 2-hydroxy-2-propyl, and R^{1b} is hydroxypentyl; R^{1a} is 2-hydroxy-2-propyl, and R^{1b} is hydroxyhexyl; R^{1b} is hydroxymethyl, and R^{1a} is hydroxymethyl; R^{1b} is hydroxymethyl, and R^{1a} is hydroxy ethyl; R^{1b} is hydroxymethyl, and R^{1a} is 2-hydroxy-2-propyl; R^{1b} is hydroxymethyl, and R^{1a} is 3-hydroxy-2-propyl; R^{1b} is hydroxymethyl, and R^{1a} is 1-hydroxy-1-propyl; R^{1b} is hydroxymethyl, and R^{1a} is 2-hydroxy-1-propyl; R^{1b} is hydroxymethyl, and R^{1a} is 3-hydroxy-1-propyl; R^{1b} is hydroxymethyl, and R^{1a} is hydroxybutyl; R^{1b} is hydroxymethyl, and R^{1a} is hydroxypentyl; R^{1b} is hydroxymethyl, and R^{1a} is hydroxyhexyl; R^{1b} is hydroxy ethyl, and R^{1a} is hydroxymethyl; R^{1b} is hydroxy ethyl, and R^{1a} is hydroxyethyl; R^{1b} is hydroxyethyl, and R^{1a} is 2-hydroxy-2-propyl; R^{1b} is hydroxyethyl, and R^{1a} is 3-hydroxy-2-propyl; R^{1b} is hydroxyethyl, and R^{1a} is 1-hydroxy-1-propyl; R^{1b} is hydroxyethyl, and R^{1a} is 2-hydroxy-1-propyl; R^{1b} is hydroxyethyl, and R^{1a} is 3-hydroxy-1-propyl; R^{1b} is hydroxyethyl, and R^{1a} is hydroxybutyl; R^{1b} is hydroxyethyl, and R^{1a} is hydroxypentyl; R^{1b} is hydroxyethyl, and R^{1a} is hydroxyhexyl; R^{1b} is 2-hydroxy-2-propyl, and R^{1a} is hydroxymethyl; R^{1b} is 2-hydroxy-2-propyl, and R^{1a} is hydroxyethyl; R^{1b} is 2-hydroxy-2-propyl, and R^{1a} is 2-hydroxy-2-propyl; R^{1b} is 2-hydroxy-2-propyl, and R^{1a} is 3-hydroxy-2-propyl; R^{1b} is 2-hydroxy-2-propyl, and R^{1a} is 1-hydroxy-1-propyl; R^{1b} is 2-hydroxy-2-propyl, and R^{1a} is 2-hydroxy-1-propyl; R^{1b} is 2-hydroxy-2-propyl, and R^{1a} is 3-hydroxy-1-propyl; R^{1b} is 2-hydroxy-2-propyl, and R^{1a} is hydroxybutyl; R^{1b} is 2-hydroxy-2-propyl, and R^{1a} is hydroxypentyl; R^{1b} is 2-hydroxy-2-propyl, and R^{1a} is hydroxyhexyl;
   and R⁶ and R⁷ are one of the following combinations:
each R⁶ is isopropyl; and R⁷ is methyl; each R⁶ is isopropyl; and R⁷ is isopropyl; each R⁶ is isopropyl; and R⁷ is trifluoromethyl; each R⁶ is isopropyl; and R⁷ is cyclopropyl; each R⁶ is isopropyl; and R⁷ is chloro; each R⁶ is isopropyl; and R⁷ is fluoro; each R⁶ is ethyl; and R⁷ is fluoro; each R⁶ is isopropyl; and R⁷ is cyano; each R⁶ is cyclopropyl; and R⁷ is cyclopropyl; each R⁶ is cyclopropyl; and R⁷ is chloro; each R⁶ is cyclopropyl; and R⁷ is fluoro; each R⁶ is isopropyl; and R⁷ is methoxy; each R⁶ is isopropyl; and R⁷ is trifluoromethoxy; each R⁶ is chloro; and R⁷ is trifluoromethyl; each R⁶ is chloro; and R⁷ is trifluoromethoxy; R⁷ is isopropyl; and each R⁶ is methyl; R⁷ is isopropyl; and each R⁶ is trifluoromethyl; R⁷ is isopropyl; and each R⁶ is cyclopropyl; R⁷ is isopropyl; and each R⁶ is chloro; R⁷ is ethyl; and each R⁶ is fluoro; R⁷ is isopropyl; and each R⁶ is cyano; R⁷ is cyclopropyl; and each R⁶ is cyclopropyl; R⁷ is cyclopropyl; and each R⁶ is chloro; R⁷ is cyclopropyl; and each R⁶ is fluoro; R⁷ is isopropyl; and each R⁶ is methoxy; R⁷ is isopropyl; and each R⁶ is trifluoromethoxy; R⁷ is chloro; and each R⁶ is trifluoromethyl; R⁷ is chloro; and each R⁶ is trifluoromethoxy; one R⁶ is isopropyl; the other R⁶ is trifluoromethyl; and R⁷ is chloro; R⁶ and R⁷ on adjacent atoms taken together with the atoms connecting them form a C₅ aliphatic carbocyclic ring; and one R⁶ is fluoro, chloro, or cyano.

In some embodiments, the compound of formula AA is a compound wherein the substituted ring

### A is

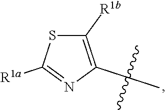

the optionally optionally substituted ring B is

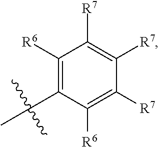

and wherein:
R^{1a} is hydroxymethyl, and R^{1b} is hydroxymethyl; R^{1a} is hydroxymethyl, and R^{1b} is hydroxy ethyl; R^{1a} is hydroxymethyl, and R^{1b} is 2-hydroxy-2-propyl; R^{1a} is hydroxymethyl, and R^{1b} is 3-hydroxy-2-propyl; R^{1a} is hydroxymethyl, and R^{1b} is 1-hydroxy-1-propyl; R^{1a} is hydroxymethyl, and R^{1b} is 2-hydroxy-1-propyl; R^{1a} is hydroxymethyl, and R^{1b} is 3-hydroxy-1-propyl; R^{1a} is hydroxymethyl, and R^{1b} is hydroxybutyl; R^{1a} is hydroxymethyl, and R^{1b} is hydroxypentyl; R^{1a} is hydroxymethyl, and R^{1b} is hydroxyhexyl; R^{1a} is hydroxy ethyl, and R^{1b} is hydroxymethyl; R^{1a} is hydroxy ethyl, and R^{1b} is hydroxy ethyl; R^{1a} is hydroxy ethyl, and R^{1b} is 2-hydroxy-2-propyl; R^{1a} is hydroxy ethyl, and R^{1b} is 3-hydroxy-2-propyl; R^{1a} is hydroxyethyl, and R^{1b} is 1-hydroxy-1-propyl; R^{1a} is hydroxyethyl, and R^{1b} is 2-hydroxy-1-propyl; R^{1a} is hydroxyethyl, and R^{1b} is 3-hydroxy-1-propyl; R^{1a} is hydroxyethyl, and R^{1b} is hydroxybutyl; R^{1a} is hydroxyethyl, and R^{1b} is hydroxypentyl; R^{1a} is hydroxyethyl, and R^{1b} is hydroxyhexyl; R^{1a} is 2-hydroxy-2-propyl, and R^{1b} is hydroxymethyl; R^{1a} is 2-hydroxy-2-propyl, and R^{1b} is hydroxyethyl; R^{1a} is 2-hydroxy-2-propyl, and R^{1b} is 2-hydroxy-2-propyl; R^{1a} is 2-hydroxy-2-propyl, and R^{1b} is 3-hydroxy-2-propyl; R^{1a} is 2-hydroxy-2-propyl, and R^{1b} is 1-hydroxy-1-propyl; R^{1a} is 2-hydroxy-2-propyl, and R^{1b} is 2-hydroxy-1-propyl; R^{1a} is 2-hydroxy-2-propyl, and R^{1b} is 3-hydroxy-1-propyl; R^{1a} is 2-hydroxy-2-propyl, and R^{1b} is hydroxybutyl; R^{1a} is 2-hydroxy-2-propyl, and R^{1b} is hydroxypentyl; R^{1a} is 2-hydroxy-2-propyl, and R^{1b} is hydroxyhexyl; R^{1b} is hydroxymethyl, and R^{1a} is hydroxymethyl; R^{1b} is hydroxymethyl, and R^{1a} is hydroxyethyl; R^{1b} is hydroxymethyl, and R^{1a} is 2-hydroxy-2-propyl; R^{1b} is hydroxymethyl, and R^{1a} is 3-hydroxy-2-propyl; R^{1b} is hydroxymethyl, and R^{1a} is 1-hydroxy-1-propyl; R^{1b} is hydroxymethyl, and R^{1a} is 2-hydroxy-1-propyl; R^{1b} is hydroxymethyl, and R^{1a} is 3-hydroxy-1-propyl; R^{1b} is hydroxymethyl, and R^{1a} is hydroxybutyl; R^{1b} is hydroxymethyl, and R^{1a} is hydroxypentyl; R^{1b} is hydroxymethyl, and R^{1a} is hydroxyhexyl; R^{1b} is hydroxy ethyl, and R^{1a} is hydroxymethyl; R^{1b} is hydroxy ethyl, and R^{1a} is hydroxyethyl; R^{1b} is hydroxyethyl, and R^{1a} is 2-hydroxy-2-propyl; R^{1b} is hydroxyethyl, and R^{1a} is 3-hydroxy-2-propyl; R^{1b} is hydroxyethyl, and R^{1a} is 1-hydroxy-1-propyl; R^{1b} is hydroxyethyl, and R^{1a} is 2-hydroxy-1-propyl; R^{1b} is hydroxyethyl, and R^{1a} is 3-hydroxy-1-propyl; R^{1b} is hydroxyethyl, and R^{1a} is hydroxybutyl; R^{1b} is hydroxyethyl, and R^{1a} is hydroxypentyl; R^{1b} is hydroxyethyl, and R^{1a} is hydroxyhexyl; R^{1b} is 2-hydroxy-2-propyl, and R^{1a} is hydroxymethyl; R^{1b} is 2-hydroxy-2-propyl, and R^{1a} is hydroxyethyl; R^{1b} is 2-hydroxy-2-propyl, and R^{1a} is 2-hydroxy-2-propyl; R^{1b} is 2-hydroxy-2-propyl, and R^{1a} is 3-hydroxy-2-propyl; R^{1b} is 2-hydroxy-2-propyl, and R^{1a} is 1-hydroxy-1-propyl; R^{1b} is 2-hydroxy-2-propyl, and R^{1a} is 2-hydroxy-1-propyl; R^{1b} is 2-hydroxy-2-propyl, and R^{1a} is 3-hydroxy-1-propyl; R^{1b} is 2-hydroxy-2-propyl, and R^{1a} is hydroxybutyl; R^{1b} is 2-hydroxy-2-propyl, and R^{1a} is hydroxypentyl; R^{1b} is 2-hydroxy-2-propyl, and R^{1a} is hydroxyhexyl;
   and R⁶ and R⁷ are one of the following combinations:
each R⁶ is independently C₁-C₆ alkyl, and R⁷ is C₁-C₆ alkyl optionally substituted with one or more halo; each R⁶ is independently C₁-C₆ alkyl and R⁷ is C₁-C₆ alkyl; each R⁶ is independently C₁-C₆ alkyl, and R⁷ is C₁-C₆ alkyl substituted with one or more halo; each R⁶ is independently C₁-C₆ alkyl, and R⁷ is C₃-C₇ cycloalkyl; each R⁶ is independently C₁-C₆ alkyl, and R⁷ is halo; each R⁶ is independently C₁-C₆ alkyl, and R⁷ is cyano; each R⁶ is independently C₃-C₇ cycloalkyl, and R⁷ is C₃-C₇ cycloalkyl; each R⁶ is independently C₃-C₇ cycloalkyl, and R⁷ is halo; each R⁶ is independently cyclopropyl and R⁷ is halo; each R⁶ is independently C₁-C₆ alkyl, and R⁷ is C₁-C₆ alkoxy optionally substituted with one or more halo; each R⁶ is independently C₁-C₆ alkyl, and R⁷ is C₁-C₆ alkoxy; each R⁶ is independently C₁-C₆ alkyl, and R⁷ is C₁-C₆ alkoxy substituted with one or more halo; each R⁶ is independently halo, and R⁷ is C₁-C₆ haloalkyl; each R⁶ is independently halo, and R⁷ is C₁-C₆ haloalkoxy; each R⁶ is independently C₁-C₆ alkoxy; and R⁷ is halo; each R⁶ is independently C₁-C₆ alkoxy; and R⁷ is chloro; R⁷ is C₁-C₆ alkyl, and each R⁶ is independently C₁-C₆ alkyl optionally substituted with one or more halo; R⁷ is C₁-C₆ alkyl, and each R⁶ is independently C₁-C₆ alkyl substituted with one or more halo; R⁷ is C₁-C₆ alkyl, and each R⁶ is independently C₃-C₇ cycloalkyl; R⁷ is C₁-C₆ alkyl, and each R⁶ is independently halo; R⁷ is C₁-C₆ alkyl and each R⁶ is independently halo; R⁷ is C₁-C₆ alkyl, and R⁶ is cyano; R⁷ is C₃-C₇ cycloalkyl, and each R⁶ is independently C₃-C₇ cycloalkyl; R⁷ is C₃-C₇ cycloalkyl, and each R⁶ is independently halo; R⁷ is C₃-C₇ cycloalkyl and each R⁶ is independently halo; R⁷ is C₁-C₆ alkyl, and each R⁶ is independently C₁-C₆ alkoxy optionally substituted with one or more halo; R⁷ is C₁-C₆ alkyl, and each R⁶ is independently C₁-C₆ alkoxy; R⁷ is C₁-C₆ alkyl, and each R⁶ is independently C₁-C₆ alkoxy substituted with one or more halo; R⁷ is halo, and each R⁶ is independently C₁-C₆ haloalkyl; R⁷ is halo, and each R⁶ is independently C₁-C₆ haloalkoxy; R⁷ is C₁-C₆ alkoxy; and each R⁶ is independently halo; R⁷ is C₁-C₆ alkoxy; and R⁶ is chloro; R⁶ and R⁷ on adjacent atoms taken together with the atoms connecting them form a C₅ aliphatic carbocyclic ring.

In some embodiments, the compound of formula AA is a compound wherein the substituted ring

### A is

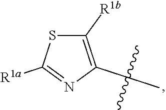

the optionally optionally substituted ring B is

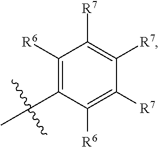

and wherein:
R^{1a} is hydroxymethyl, and R^{1b} is hydroxymethyl; R^{1a} is hydroxymethyl, and R^{1b} is hydroxy ethyl; R^{1a} is hydroxymethyl, and R^{1b} is 2-hydroxy-2-propyl; R^{1a} is hydroxymethyl, and R^{1b} is 3-hydroxy-2-propyl; R^{1a} is hydroxymethyl, and R^{1b} is 1-hydroxy-1-propyl; R^{1a} is hydroxymethyl, and R^{1b} is 2-hydroxy-1-propyl; R^{1a} is hydroxymethyl, and R^{1b} is 3-hydroxy-1-propyl; R^{1a} is hydroxymethyl, and R^{1b} is hydroxybutyl; R^{1a} is hydroxymethyl, and R^{1b} is hydroxypentyl; R^{1a} is hydroxymethyl, and R^{1b} is hydroxyhexyl; R^{1a} is hydroxy ethyl, and R^{1b} is hydroxymethyl; R^{1a} is hydroxy ethyl, and R^{1b} is hydroxy ethyl; R^{1a} is hydroxy ethyl, and R^{1b} is 2-hydroxy-2-propyl; R^{1a} is hydroxy ethyl, and R^{1b} is 3-hydroxy-2-propyl; R^{1a} is hydroxyethyl, and R^{1b} is 1-hydroxy-1-propyl; R^{1a} is hydroxyethyl, and R^{1b} is 2-hydroxy-1-propyl; R^{1a} is hydroxyethyl, and R^{1b} is 3-hydroxy-1-propyl; R^{1a} is hydroxyethyl, and R^{1b} is hydroxybutyl; R^{1a} is hydroxyethyl, and R^{1b} is hydroxypentyl; R^{1a} is hydroxyethyl, and R^{1b} is hydroxyhexyl; R^{1a} is 2-hydroxy-2-propyl, and R^{1b} is hydroxymethyl; R^{1a} is 2-hydroxy-2-propyl, and R^{1b} is hydroxyethyl; R^{1a} is 2-hydroxy-2-propyl, and R^{1b} is 2-hydroxy-2-propyl; R^{1a} is 2-hydroxy-2-propyl, and R^{1b} is 3-hydroxy-2-propyl; R^{1a} is 2-hydroxy-2-propyl, and R^{1b} is 1-hydroxy-1-propyl; R^{1a} is 2-hydroxy-2-propyl, and R^{1b} is 2-hydroxy-1-propyl; R^{1a} is 2-hydroxy-2-propyl, and R^{1b} is 3-hydroxy-1-propyl; R^{1a} is 2-hydroxy-2-propyl, and R^{1b} is hydroxybutyl; R^{1a} is 2-hydroxy-2-propyl, and R^{1b} is hydroxypentyl; R^{1a} is 2-hydroxy-2-propyl, and R^{1b} is hydroxyhexyl; R^{1b} is hydroxymethyl, and R^{1a} is hydroxymethyl; R^{1b} is hydroxymethyl, and R^{1a} is hydroxy ethyl; R^{1b} is hydroxymethyl, and R^{1a} is 2-hydroxy-2-propyl; R^{1b} is hydroxymethyl, and R^{1a} is 3-hydroxy-2-propyl; R^{1b} is hydroxymethyl, and R^{1a} is 1-hydroxy-1-propyl; R^{1b} is hydroxymethyl, and R^{1a} is 2-hydroxy-1-propyl; R^{1b} is hydroxymethyl, and R^{1a} is 3-hydroxy-1-propyl; R^{1b} is hydroxymethyl, and R^{1a} is hydroxybutyl; R^{1b} is hydroxymethyl, and R^{1a} is hydroxypentyl; R^{1b} is hydroxymethyl, and R^{1a} is hydroxyhexyl; R^{1b} is hydroxy ethyl, and R^{1a} is hydroxymethyl; R^{1b} is hydroxy ethyl, and R^{1a} is hydroxyethyl; R^{1b} is hydroxyethyl, and R^{1a} is 2-hydroxy-2-propyl; R^{1b} is hydroxyethyl, and R^{1a} is 3-hydroxy-2-propyl; R^{1b} is hydroxyethyl, and R^{1a} is 1-hydroxy-1-propyl; R^{1b} is hydroxyethyl, and R^{1a} is 2-hydroxy-1-propyl; R^{1b} is hydroxyethyl, and R^{1a} is 3-hydroxy-1-propyl; R^{1b} is hydroxyethyl, and R^{1a} is hydroxybutyl; R^{1b} is hydroxyethyl, and R^{1a} is hydroxypentyl; R^{1b} is hydroxyethyl, and R^{1a} is hydroxyhexyl; R^{1b} is 2-hydroxy-2-propyl, and R^{1a} is hydroxymethyl; R^{1b} is 2-hydroxy-2-propyl, and R^{1a} is hydroxyethyl; R^{1b} is 2-hydroxy-2-propyl, and R^{1a} is 2-hydroxy-2-propyl; R^{1b} is 2-hydroxy-2-propyl, and R^{1a} is 3-hydroxy-2-propyl; R^{1b} is 2-hydroxy-2-propyl, and R^{1a} is 1-hydroxy-1-propyl; R^{1b} is 2-hydroxy-2-propyl, and R^{1a} is 2-hydroxy-1-propyl; R^{1b} is 2-hydroxy-2-propyl, and R^{1a} is 3-hydroxy-1-propyl; R^{1b} is 2-hydroxy-2-propyl, and R^{1a} is hydroxybutyl; R^{1b} is 2-hydroxy-2-propyl, and R^{1a} is hydroxypentyl; R^{1b} is 2-hydroxy-2-propyl, and R^{1a} is hydroxyhexyl;
   and R⁶ and R⁷ are one of the following combinations:
each R⁶ is isopropyl; and R⁷ is methyl; each R⁶ is isopropyl; and R⁷ is isopropyl; each R⁶ is isopropyl; and R⁷ is trifluoromethyl; each R⁶ is isopropyl; and R⁷ is cyclopropyl; each R⁶ is isopropyl; and R⁷ is chloro; each R⁶ is isopropyl; and R⁷ is fluoro; each R⁶ is ethyl; and R⁷ is fluoro; each R⁶ is isopropyl; and R⁷ is cyano; each R⁶ is cyclopropyl; and R⁷ is cyclopropyl; each R⁶ is cyclopropyl; and R⁷ is chloro; each R⁶ is cyclopropyl; and R⁷ is fluoro; each R⁶ is isopropyl; and R⁷ is methoxy; each R⁶ is isopropyl; and R⁷ is trifluoromethoxy; each R⁶ is chloro; and R⁷ is trifluoromethyl; each R⁶ is chloro; and R⁷ is trifluoromethoxy; R⁷ is isopropyl; and each R⁶ is methyl; R⁷ is isopropyl; and each R⁶ is trifluoromethyl; R⁷ is isopropyl; and each R⁶ is cyclopropyl; R⁷ is isopropyl; and each R⁶ is chloro; R⁷ is ethyl; and each R⁶ is fluoro; R⁷ is isopropyl; and each R⁶ is cyano; R⁷ is cyclopropyl; and each R⁶ is cyclopropyl; R⁷ is cyclopropyl; and each R⁶ is chloro; R⁷ is cyclopropyl; and each R⁶ is fluoro; R⁷ is isopropyl; and each R⁶ is methoxy; R⁷ is isopropyl; and each R⁶ is trifluoromethoxy; R⁷ is chloro; and each R⁶ is trifluoromethyl; R⁷ is chloro; and each R⁶ is trifluoromethoxy; one R⁶ is isopropyl; the other R⁶ is trifluoromethyl; and R⁷ is chloro; R⁶ and R⁷ on adjacent atoms taken together with the atoms connecting them form a C₅ aliphatic carbocyclic ring; and one R⁶ is fluoro, chloro, or cyano.

### Additional Features of the Embodiments Herein

In some embodiments, the compound of Formula AA is not a compound selected from the group consisting of:

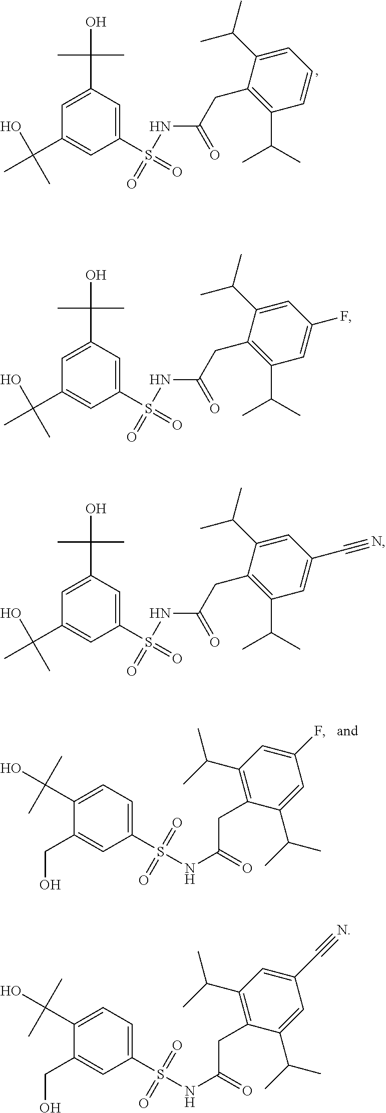 [SMILES: CC(C)c1cccc(C(C)C)c1CC(=O)NS(=O)(=O)c1cc(C(C)(C)O)cc(C(C)(C)O)c1 ; CC(C)c1cc(F)cc(C(C)C)c1CC(=O)NS(=O)(=O)c1cc(C(C)(C)O)cc(C(C)(C)O)c1 ; CC(C)c1cc(C#N)cc(C(C)C)c1CC(=O)NS(=O)(=O)c1cc(C(C)(C)O)cc(C(C)(C)O)c1 ; CC(C)c1cc(F)cc(C(C)C)c1CC(=O)NS(=O)(=O)c1ccc(C(C)(C)O)c(CO)c1 ; "and" ; CC(C)c1cc(C#N)cc(C(C)C)c1CC(=O)NS(=O)(=O)c1ccc(C(C)(C)O)c(CO)c1]

In some embodiments, the compound of Formula AA is not a compound selected from the group consisting of:

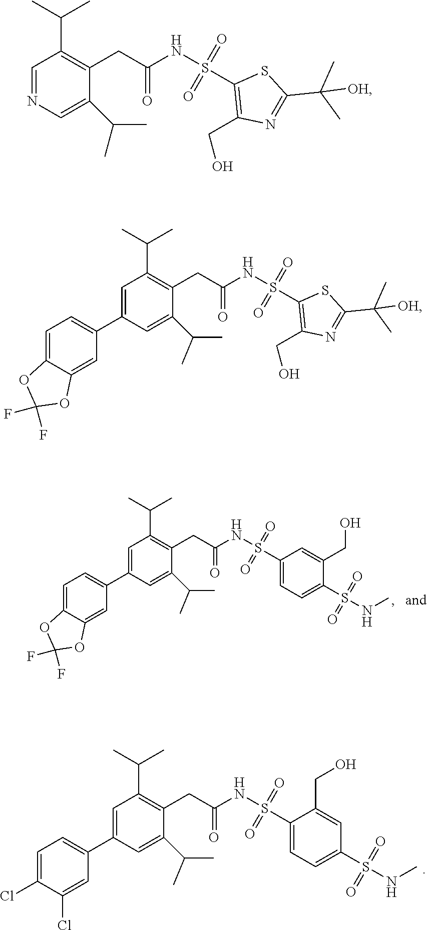 [SMILES: CC(C)c1cncc(C(C)C)c1CC(=O)NS(=O)(=O)c1sc(C(C)(C)O)nc1CO ; CC(C)c1cc(-c2ccc3c(c2)OC(F)(F)O3)cc(C(C)C)c1CC(=O)NS(=O)(=O)c1sc(C(C)(C)O)nc1CO ; CNS(=O)(=O)c1ccc(S(=O)(=O)NC(=O)Cc2c(C(C)C)cc(-c3ccc4c(c3)OC(F)(F)O4)cc2C(C)C)cc1CO ; CNS(=O)(=O)c1ccc(S(=O)(=O)NC(=O)Cc2c(C(C)C)cc(-c3ccc(Cl)c(Cl)c3)cc2C(C)C)c(CO)c1]

In some embodiments the compound of any of the formulae herein is not a compound disclosed in patent publication WO2017/184604 (e.g., compounds 101-215).

Unless otherwise indicated, when a disclosed compound is named or depicted by a structure without specifying the stereochemistry and has one or more chiral centers, it is understood to represent all possible stereoisomers of the compound.

It is understood that the combination of variables in the formulae herein is such that the compounds are stable.

In some embodiments, provided herein is a compound that is selected from the group consisting of the compounds in Table 1 A:

TABLE 1A

| Compound | Structure | m/z |
|---|---|---|
| 101 | 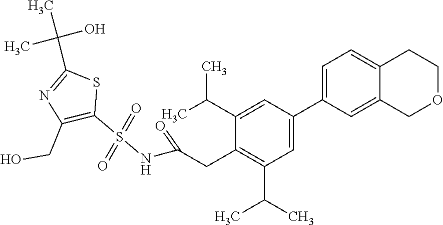 [SMILES: CC(C)c1cc(-c2ccc3c(c2)COCC3)cc(C(C)C)c1CC(=O)NS(=O)(=O)c1sc(C(C)(C)O)nc1CO] | 587.2 |
| 102 | 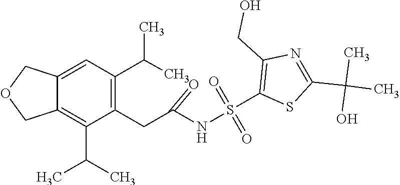 [SMILES: CC(C)c1cc2c(c(C(C)C)c1CC(=O)NS(=O)(=O)c1sc(C(C)(C)O)nc1CO)COC2] | 497.2 |
| 103 | 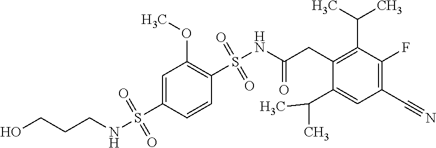 [SMILES: COc1cc(S(=O)(=O)NCCCO)ccc1S(=O)(=O)NC(=O)Cc1c(C(C)C)cc(C#N)c(F)c1C(C)C] | 570 |
| 104 | 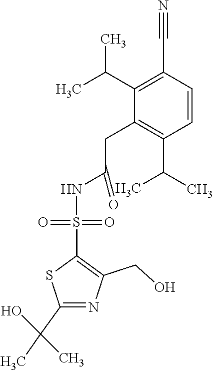 [SMILES: CC(C)c1ccc(C#N)c(C(C)C)c1CC(=O)NS(=O)(=O)c1sc(C(C)(C)O)nc1CO] | 480.1 |
| 105 | 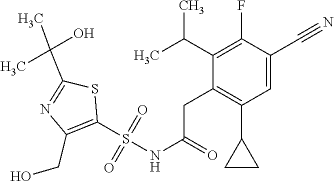 [SMILES: CC(C)c1c(F)c(C#N)cc(C2CC2)c1CC(=O)NS(=O)(=O)c1sc(C(C)(C)O)nc1CO] | 496.1 |
| 106 | 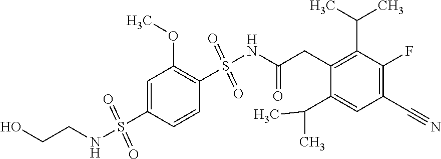 [SMILES: COc1cc(S(=O)(=O)NCCO)ccc1S(=O)(=O)NC(=O)Cc1c(C(C)C)cc(C#N)c(F)c1C(C)C] | 555.1 |
| 107 | 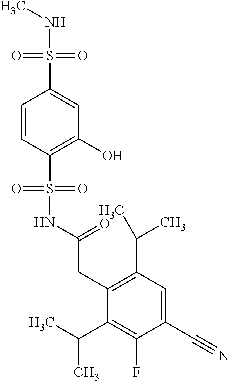 [SMILES: CNS(=O)(=O)c1ccc(S(=O)(=O)NC(=O)Cc2c(C(C)C)cc(C#N)c(F)c2C(C)C)c(O)c1] | 512.2 |
| 108 | 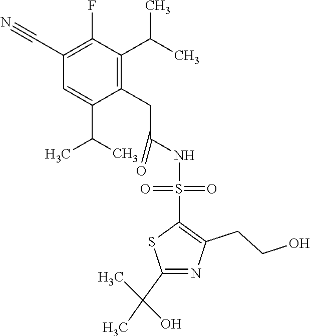 [SMILES: CC(C)c1cc(C#N)c(F)c(C(C)C)c1CC(=O)NS(=O)(=O)c1sc(C(C)(C)O)nc1CCO] | 512.3 |
| 109 | 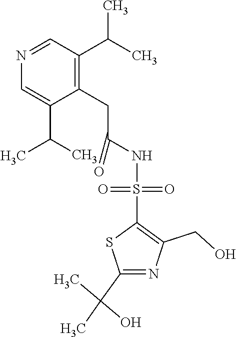 [SMILES: CC(C)c1cncc(C(C)C)c1CC(=O)NS(=O)(=O)c1sc(C(C)(C)O)nc1CO] | 455.15 |
| 110 | 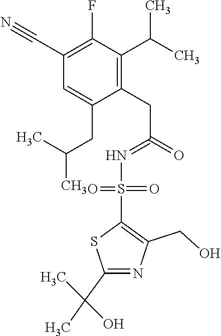 | 510.2 |
| 111 | 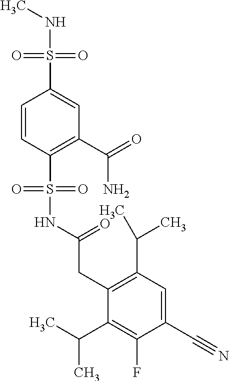 [SMILES: CNS(=O)(=O)c1ccc(S(=O)(=O)NC(=O)Cc2c(C(C)C)cc(C#N)c(F)c2C(C)C)c(C(N)=O)c1] | 538.1 |
| 112 | 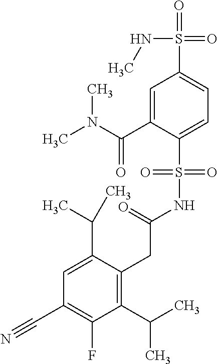 [SMILES: CNS(=O)(=O)c1ccc(S(=O)(=O)NC(=O)Cc2c(C(C)C)cc(C#N)c(F)c2C(C)C)c(C(=O)N(C)C)c1] | 567.3 |
| 114 | 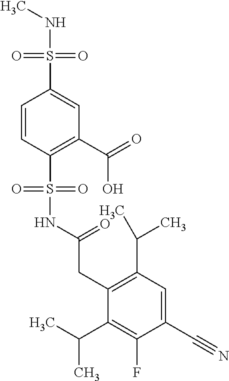 [SMILES: CNS(=O)(=O)c1ccc(S(=O)(=O)NC(=O)Cc2c(C(C)C)cc(C#N)c(F)c2C(C)C)c(C(=O)O)c1] | 538.1 |
| 115 | 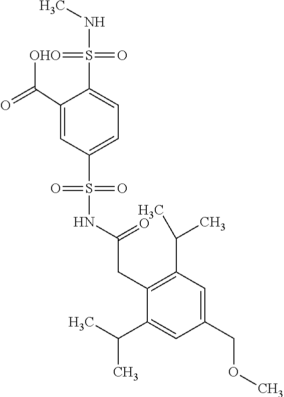 | 540.1 |
| 116 | 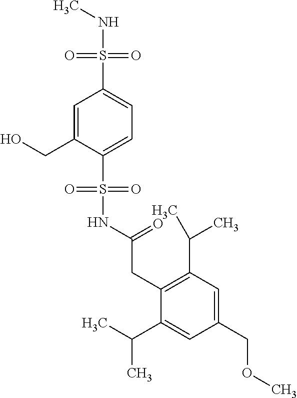 [SMILES: CNS(=O)(=O)c1ccc(S(=O)(=O)NC(=O)Cc2c(C(C)C)cc(COC)cc2C(C)C)c(CO)c1] | 526.1 |
| 117 | 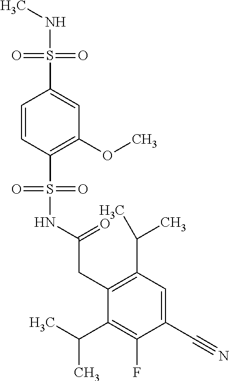 [SMILES: CNS(=O)(=O)c1ccc(S(=O)(=O)NC(=O)Cc2c(C(C)C)cc(C#N)c(F)c2C(C)C)c(OC)c1] | 524.0 |
| 118 | 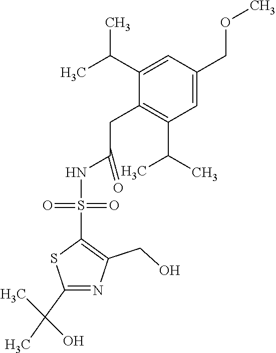 [SMILES: COCc1cc(C(C)C)c(CC(=O)NS(=O)(=O)c2sc(C(C)(C)O)nc2CO)c(C(C)C)c1] | 497.1 |
| 119 | 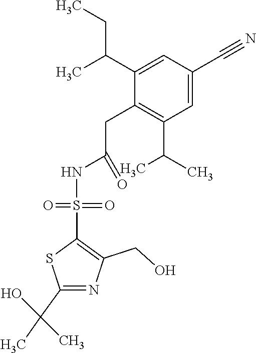 [SMILES: CCC(C)c1cc(C#N)cc(C(C)C)c1CC(=O)NS(=O)(=O)c1sc(C(C)(C)O)nc1CO] | 493.1 |
| 120 | 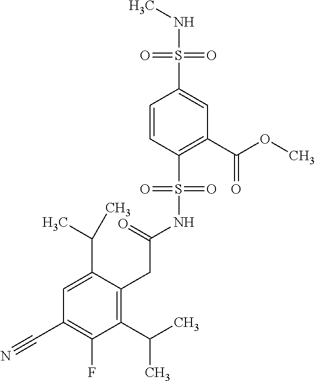 [SMILES: CNS(=O)(=O)c1ccc(S(=O)(=O)NC(=O)Cc2c(C(C)C)cc(C#N)c(F)c2C(C)C)c(C(=O)OC)c1] | 553.1 |
| 121 | 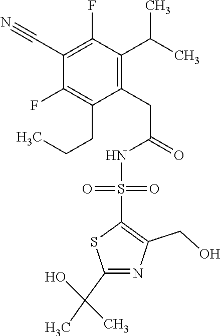 [SMILES: CCCc1c(F)c(C#N)c(F)c(C(C)C)c1CC(=O)NS(=O)(=O)c1sc(C(C)(C)O)nc1CO] | 497.1 |
| 122 | 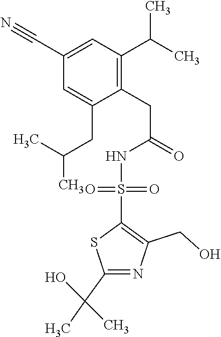 [SMILES: CC(C)Cc1cc(C#N)cc(C(C)C)c1CC(=O)NS(=O)(=O)c1sc(C(C)(C)O)nc1CO] | 493.6 |
| 123 | 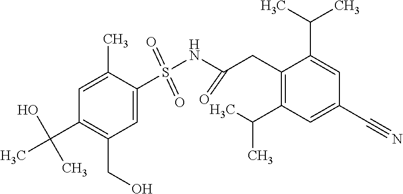 [SMILES: Cc1cc(C(C)(C)O)c(CO)cc1S(=O)(=O)NC(=O)Cc1c(C(C)C)cc(C#N)cc1C(C)C] | 486.6 |
| 124 | 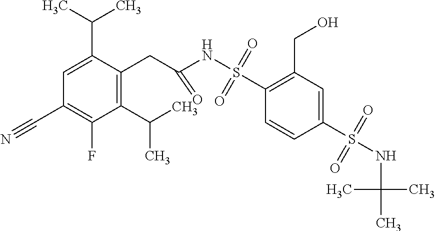 [SMILES: CC(C)c1cc(C#N)c(F)c(C(C)C)c1CC(=O)NS(=O)(=O)c1ccc(S(=O)(=O)NC(C)(C)C)cc1CO] | 567.1 |
| 125 | 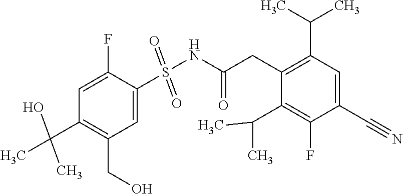 [SMILES: CC(C)c1cc(C#N)c(F)c(C(C)C)c1CC(=O)NS(=O)(=O)c1cc(CO)c(C(C)(C)O)cc1F] | 508.1 |
| 126 | 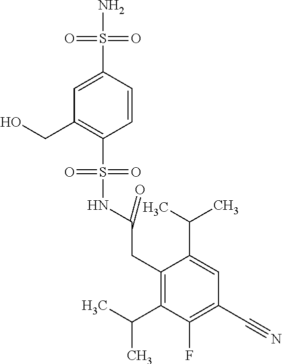 [SMILES: CC(C)c1cc(C#N)c(F)c(C(C)C)c1CC(=O)NS(=O)(=O)c1ccc(S(N)(=O)=O)cc1CO] | 511.1 |
| 127 | 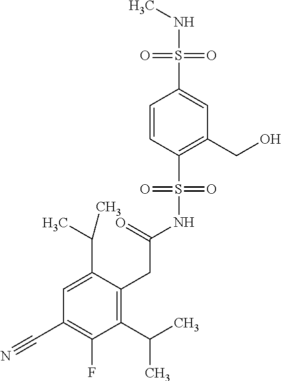 [SMILES: CNS(=O)(=O)c1ccc(S(=O)(=O)NC(=O)Cc2c(C(C)C)cc(C#N)c(F)c2C(C)C)c(CO)c1] | 525.1 |
| 128 | 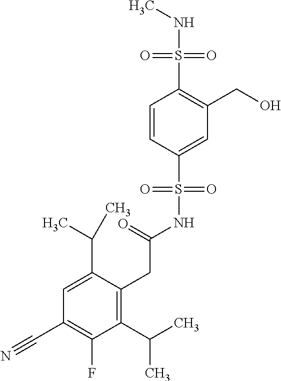 [SMILES: CNS(=O)(=O)c1ccc(S(=O)(=O)NC(=O)Cc2c(C(C)C)cc(C#N)c(F)c2C(C)C)cc1CO] | 525.1 |
| 129 | 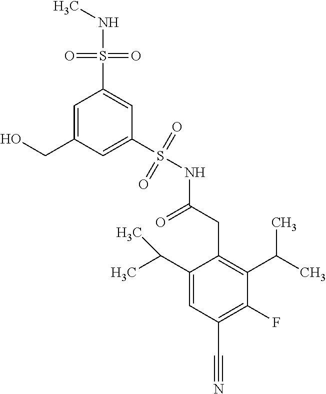 [SMILES: CNS(=O)(=O)c1cc(CO)cc(S(=O)(=O)NC(=O)Cc2c(C(C)C)cc(C#N)c(F)c2C(C)C)c1] | 525.1 |
| 130 | 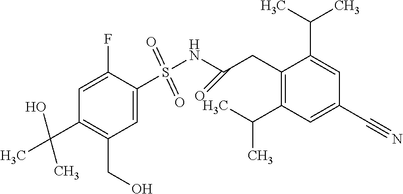 [SMILES: CC(C)c1cc(C#N)cc(C(C)C)c1CC(=O)NS(=O)(=O)c1cc(CO)c(C(C)(C)O)cc1F] | 490.1 |
| 131 | 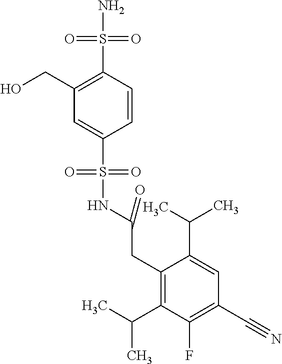 [SMILES: CC(C)c1cc(C#N)c(F)c(C(C)C)c1CC(=O)NS(=O)(=O)c1ccc(S(N)(=O)=O)c(CO)c1] | 511.1 |
| 132 | 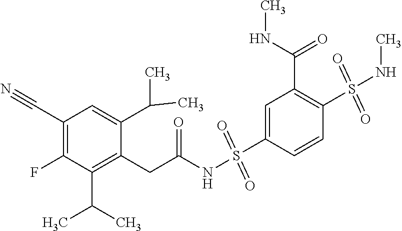 [SMILES: CNC(=O)c1cc(S(=O)(=O)NC(=O)Cc2c(C(C)C)cc(C#N)c(F)c2C(C)C)ccc1S(=O)(=O)NC] | 552.1 |
| 133 | 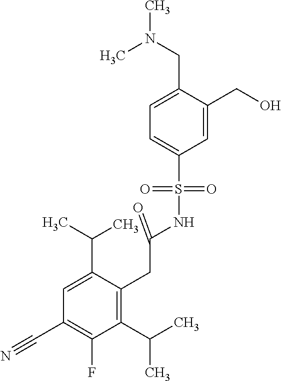 [SMILES: CC(C)c1cc(C#N)c(F)c(C(C)C)c1CC(=O)NS(=O)(=O)c1ccc(CN(C)C)c(CO)c1] | 489.1 |
| 134 | 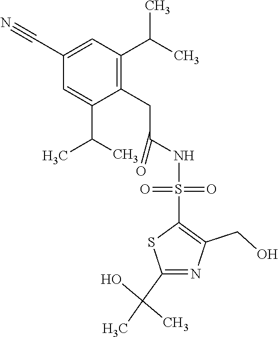 [SMILES: CC(C)c1cc(C#N)cc(C(C)C)c1CC(=O)NS(=O)(=O)c1sc(C(C)(C)O)nc1CO] | 497.1 |
| 135 | 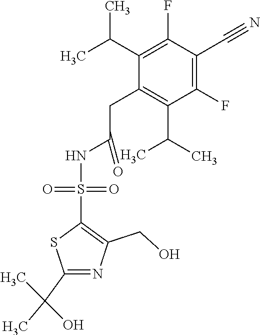 [SMILES: CC(C)c1c(F)c(C#N)c(F)c(C(C)C)c1CC(=O)NS(=O)(=O)c1sc(C(C)(C)O)nc1CO] | 515.1 |
| 136 | 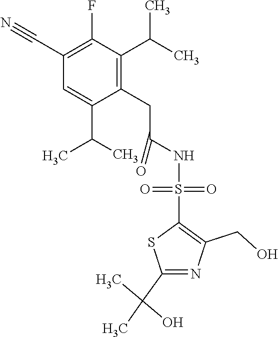 [SMILES: CC(C)c1cc(C#N)c(F)c(C(C)C)c1CC(=O)NS(=O)(=O)c1sc(C(C)(C)O)nc1CO] | 497.1 |
| 137 | 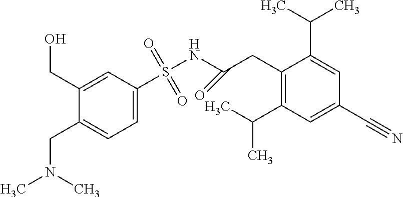 [SMILES: CC(C)c1cc(C#N)cc(C(C)C)c1CC(=O)NS(=O)(=O)c1ccc(CN(C)C)c(CO)c1] | 471.1 |
| 138 | 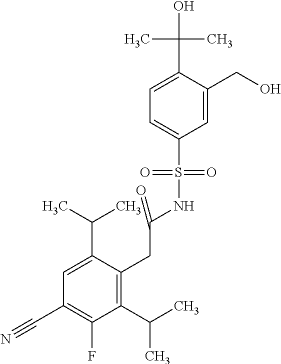 [SMILES: CC(C)c1cc(C#N)c(F)c(C(C)C)c1CC(=O)NS(=O)(=O)c1ccc(C(C)(C)O)c(CO)c1] | 490.1 |
| 139 | 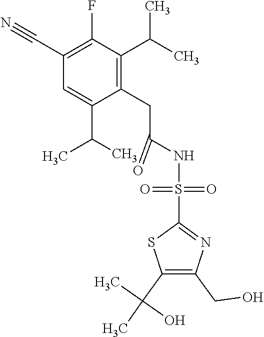 [SMILES: CC(C)c1cc(C#N)c(F)c(C(C)C)c1CC(=O)NS(=O)(=O)c1nc(CO)c(C(C)(C)O)s1] | 497.1 |
| 140 | 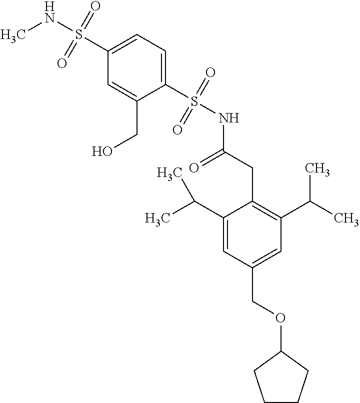 [SMILES: CNS(=O)(=O)c1ccc(S(=O)(=O)NC(=O)Cc2c(C(C)C)cc(COC3CCCC3)cc2C(C)C)c(CO)c1] | 581.3 |
| 141 | 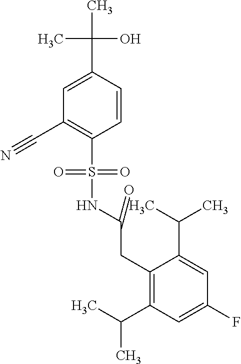 [SMILES: CC(C)c1cc(F)cc(C(C)C)c1CC(=O)NS(=O)(=O)c1ccc(C(C)(C)O)cc1C#N] | 460.1 |
| 142 | 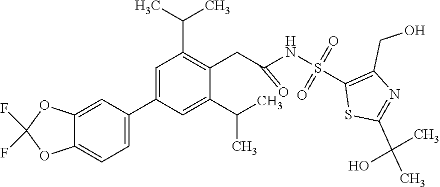 [SMILES: CC(C)c1cc(-c2ccc3c(c2)OC(F)(F)O3)cc(C(C)C)c1CC(=O)NS(=O)(=O)c1sc(C(C)(C)O)nc1CO] | 610.16 |

and pharmaceutically acceptable salts thereof.

In some embodiments, provided herein is a compound that is selected from the group consisting of the compounds in Table IB:

TABLE 1B

| Compound | Structure |
|---|---|
| 201 | 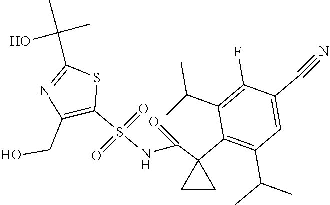 [SMILES: CC(C)c1cc(C#N)c(F)c(C(C)C)c1C1(C(=O)NS(=O)(=O)c2sc(C(C)(C)O)nc2CO)CC1] |
| 202 | 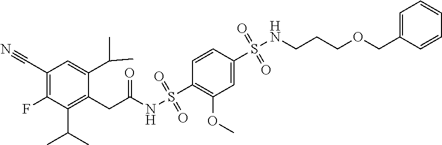 [SMILES: COc1cc(S(=O)(=O)NCCCOCc2ccccc2)ccc1S(=O)(=O)NC(=O)Cc1c(C(C)C)cc(C#N)c(F)c1C(C)C] |

and pharmaceutically acceptable salts thereof.

Pharmaceutical Compositions and Administration

General

In some embodiments, a chemical entity (e.g., a compound that modulates (e.g., antagonizes) NLRP3, or a pharmaceutically acceptable salt, and/or hydrate, and/or cocrystal, and/or drug combination thereof) is administered as a pharmaceutical composition that includes the chemical entity and one or more pharmaceutically acceptable excipients, and optionally one or more additional therapeutic agents as described herein.

In some embodiments, the chemical entities can be administered in combination with one or more conventional pharmaceutical excipients. Pharmaceutically acceptable excipients include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β, and γ-cyclodextrin, or chemically modified derivatives such as hydroxyalkylcyclodextrins, including 2- and 3-hydroxypropyl-β-cyclodextrins, or other solubilized derivatives can also be used to enhance delivery of compounds described herein. Dosage forms or compositions containing a chemical entity as described herein in the range of 0.005% to 100% with the balance made up from non-toxic excipient may be prepared. The contemplated compositions may contain 0.001%-100% of a chemical entity provided herein, in one embodiment 0.1-95%, in another embodiment 75-85%, in a further embodiment 20-80%. Actual methods of preparing such dosage forms are known, or will be apparent, to those skilled in this art; for example, see Remington; The Science and Practice of Pharmacy, 22^{nd} Edition (Pharmaceutical Press, London, U K. 2012).

Routes of Administration and Composition Components

In some embodiments, the chemical entities described herein or a pharmaceutical composition thereof can be administered to subject in need thereof by any accepted route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracistemal, intracoronary, intradermal, intraductal, intraduodenal, intradural, intraepidermal, intraesophageal, intragastric, intragingival, intraileal, intralymphatic, intramedullary, intrameningeal, intramuscular, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal. In certain embodiments, a preferred route of administration is parenteral (e.g., intratumoral).

Compositions can be formulated for parenteral administration, e.g., formulated for injection via the intravenous, intramuscular, sub-cutaneous, or even intraperitoneal routes. Typically, such compositions can be prepared as injectables, either as liquid solutions or suspensions; solid forms suitable for use to prepare solutions or suspensions upon the addition of a liquid prior to injection can also be prepared; and the preparations can also be emulsified. The preparation of such formulations will be known to those of skill in the art in light of the present disclosure.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Intratumoral injections are discussed, e.g., in Lammers, et al., “Effect of Intratumoral Injection on the Biodistribution and the Therapeutic Potential of HPMA Copolymer-Based Drug Delivery Systems” Neoplasia. 2006, 10, 788-795.

In certain embodiments, the chemical entities described herein or a pharmaceutical composition thereof are suitable for local, topical administration to the digestive or GI tract, e.g., rectal administration. Rectal compositions include, without limitation, enemas, rectal gels, rectal foams, rectal aerosols, suppositories, jelly suppositories, and enemas (e.g., retention enemas).

Pharmacologically acceptable excipients usable in the rectal composition as a gel, cream, enema, or rectal suppository, include, without limitation, any one or more of cocoa butter glycerides, synthetic polymers such as polyvinylpyrrolidone, PEG (like PEG ointments), glycerine, glycerinated gelatin, hydrogenated vegetable oils, poloxamers, mixtures of polyethylene glycols of various molecular weights and fatty acid esters of polyethylene glycol Vaseline, anhydrous lanolin, shark liver oil, sodium saccharinate, menthol, sweet almond oil, sorbitol, sodium benzoate, anoxid SBN, vanilla essential oil, aerosol, parabens in phenoxyethanol, sodium methyl p-oxybenzoate, sodium propyl p-oxybenzoate, diethylamine, carbomers, carbopol, methyloxybenzoate, macrogol cetostearyl ether, cocoyl caprylocaprate, isopropyl alcohol, propylene glycol, liquid paraffin, xanthan gum, carboxy-metabisulfite, sodium edetate, sodium benzoate, potassium metabisulfite, grapefruit seed extract, methyl sulfonyl methane (MSM), lactic acid, glycine, vitamins, such as vitamin A and E and potassium acetate.

In certain embodiments, suppositories can be prepared by mixing the chemical entities described herein with suitable non-irritating excipients or carriers such as cocoa butter, polyethylene glycol or a suppository wax which are solid at ambient temperature but liquid at body temperature and therefore melt in the rectum and release the active compound. In other embodiments, compositions for rectal administration are in the form of an enema.

In other embodiments, the compounds described herein or a pharmaceutical composition thereof are suitable for local delivery to the digestive or GI tract by way of oral administration (e.g., solid or liquid dosage forms.).

Solid dosage forms for oral administration include capsules, tablets, pills, powders, and granules. In such solid dosage forms, the chemical entity is mixed with one or more pharmaceutically acceptable excipients, such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

In one embodiment, the compositions will take the form of a unit dosage form such as a pill or tablet and thus the composition may contain, along with a chemical entity provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils, PEG'S, poloxamer 124 or triglycerides) is encapsulated in a capsule (gelatin or cellulose base capsule). Unit dosage forms in which one or more chemical entities provided herein or additional active agents are physically separated are also contemplated; e.g, capsules with granules (or tablets in a capsule) of each drug; two-layer tablets; two-compartment gel caps, etc. Enteric coated or delayed release oral dosage forms are also contemplated.

Other physiologically acceptable compounds include wetting agents, emulsifying agents, dispersing agents or preservatives that are particularly useful for preventing the growth or action of microorganisms. Various preservatives are well known and include, for example, phenol and ascorbic acid.

In certain embodiments the excipients are sterile and generally free of undesirable matter. These compositions can be sterilized by conventional, well-known sterilization techniques. For various oral dosage form excipients such as tablets and capsules sterility is not required. The USP/NF standard is usually sufficient.

In certain embodiments, solid oral dosage forms can further include one or more components that chemically and/or structurally predispose the composition for delivery of the chemical entity to the stomach or the lower GI; e.g., the ascending colon and/or transverse colon and/or distal colon and/or small bowel. Exemplary formulation techniques are described in, e.g., Filipski, K. J., et al., Current Topics in Medicinal Chemistry, 2013, 13, 776-802, which is incorporated herein by reference in its entirety.

Examples include upper-GI targeting techniques, e.g., Accordion Pill (Intec Pharma), floating capsules, and materials capable of adhering to mucosal walls.

Other examples include lower-GI targeting techniques. For targeting various regions in the intestinal tract, several enteric/pH-responsive coatings and excipients are available. These materials are typically polymers that are designed to dissolve or erode at specific pH ranges, selected based upon the GI region of desired drug release. These materials also function to protect acid labile drugs from gastric fluid or limit exposure in cases where the active ingredient may be irritating to the upper GI (e.g., hydroxypropyl methylcellulose phthalate series, Coateric (polyvinyl acetate phthalate), cellulose acetate phthalate, hydroxypropyl methylcellulose acetate succinate, Eudragit series (methacrylic acid-methyl methacrylate copolymers), and Marcoat). Other techniques include dosage forms that respond to local flora in the GI tract, Pressure-controlled colon delivery capsule, and Pulsincap.

Ocular compositions can include, without limitation, one or more of any of the following: viscogens (e.g., Carboxymethylcellulose, Glycerin, Polyvinylpyrrolidone, Polyethylene glycol); Stabilizers (e.g., Pluronic (triblock copolymers), Cyclodextrins); Preservatives (e.g., Benzalkonium chloride, ETDA, SofZia (boric acid, propylene glycol, sorbitol, and zinc chloride; Alcon Laboratories, Inc.), Purite (stabilized oxychloro complex; Allergan, Inc.)).

Topical compositions can include ointments and creams. Ointments are semisolid preparations that are typically based on petrolatum or other petroleum derivatives. Creams containing the selected active agent are typically viscous liquid or semisolid emulsions, often either oil-in-water or water-in-oil. Cream bases are typically water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. As with other carriers or vehicles, an ointment base should be inert, stable, nonirritating and non-sensitizing.

In any of the foregoing embodiments, pharmaceutical compositions described herein can include one or more one or more of the following: lipids, interbilayer crosslinked multilamellar vesicles, biodegradeable poly(D,L-lactic-co-glycolic acid) [PLGA]-based or poly anhydride-based nanoparticles or microparticles, and nanoporous particle-supported lipid bilayers.

Enema Formulations

In some embodiments, enema formulations containing the chemical entities described herein are provided in “ready-to-use” form.

In some embodiments, enema formulations containing the chemical entities described herein are provided in one or more kits or packs. In certain embodiments, the kit or pack includes two or more separately contained/packaged components, e.g. two components, which when mixed together, provide the desired formulation (e.g., as a suspension). In certain of these embodiments, the two component system includes a first component and a second component, in which: (i) the first component (e.g., contained in a sachet) includes the chemical entity (as described anywhere herein) and optionally one or more pharmaceutically acceptable excipients (e.g., together formulated as a solid preparation, e.g., together formulated as a wet granulated solid preparation); and (ii) the second component (e.g., contained in a vial or bottle) includes one or more liquids and optionally one or more other pharmaceutically acceptable excipients together forming a liquid carrier. Prior to use (e.g., immediately prior to use), the contents of (i) and (ii) are combined to form the desired enema formulation, e.g., as a suspension. In other embodiments, each of component (i) and (ii) is provided in its own separate kit or pack.

In some embodiments, each of the one or more liquids is water, or a physiologically acceptable solvent, or a mixture of water and one or more physiologically acceptable solvents. Typical such solvents include, without limitation, glycerol, ethylene glycol, propylene glycol, polyethylene glycol and polypropylene glycol. In certain embodiments, each of the one or more liquids is water. In other embodiments, each of the one or more liquids is an oil, e.g. natural and/or synthetic oils that are commonly used in pharmaceutical preparations.

Further pharmaceutical excipients and carriers that may be used in the pharmaceutical products herein described are listed in various handbooks (e.g. D. E. Bugay and W. P. Findlay (Eds) Pharmaceutical excipients (Marcel Dekker, New York, 1999), E-M Hoepfner, A. Reng and P. C. Schmidt (Eds) Fiedler Encyclopedia of Excipients for Pharmaceuticals, Cosmetics and Related Areas (Edition Cantor, Munich, 2002) and H. P. Fielder (Ed) Lexikon der Hilfsstoffe fur Pharmazie, Kosmetik and angrenzende Gebiete (Edition Cantor Aulendorf, 1989)).

In some embodiments, each of the one or more pharmaceutically acceptable excipients can be independently selected from thickeners, viscosity enhancing agents, bulking agents, mucoadhesive agents, penetration enhanceers, buffers, preservatives, diluents, binders, lubricants, glidants, disintegrants, fillers, solubilizing agents, pH modifying agents, preservatives, stabilizing agents, anti-oxidants, wetting or emulsifying agents, suspending agents, pigments, colorants, isotonic agents, chelating agents, emulsifiers, and diagnostic agents.

In certain embodiments, each of the one or more pharmaceutically acceptable excipients can be independently selected from thickeners, viscosity enhancing agents, mucoadhesive agents, buffers, preservatives, diluents, binders, lubricants, glidants, disintegrants, and fillers.

In certain embodiments, each of the one or more pharmaceutically acceptable excipients can be independently selected from thickeners, viscosity enhancing agents, bulking agents, mucoadhesive agents, buffers, preservatives, and fillers.

In certain embodiments, each of the one or more pharmaceutically acceptable excipients can be independently selected from diluents, binders, lubricants, glidants, and disintegrants.

Examples of thickeners, viscosity enhancing agents, and mucoadhesive agents include without limitation: gums, e.g. xanthan gum, guar gum, locust bean gum, tragacanth gums, karaya gum, ghatti gum, cholla gum, psyllium seed gum and gum arabic; poly(carboxylic acid-containing) based polymers, such as poly (acrylic, maleic, itaconic, citraconic, hydroxyethyl methacrylic or methacrylic) acid which have strong hydrogen-bonding groups, or derivatives thereof such as salts and esters; cellulose derivatives, such as methyl cellulose, ethyl cellulose, methylethyl cellulose, hydroxymethyl cellulose, hydroxyethyl cellulose, hydroxypropyl cellulose, hydroxyethyl ethyl cellulose, carboxymethyl cellulose, hydroxypropylmethyl cellulose or cellulose esters or ethers or derivatives or salts thereof; clays such as manomorillonite clays, e.g. Veegun, attapulgite clay; polysaccharides such as dextran, pectin, amylopectin, agar, mannan or polygalactonic acid or starches such as hydroxypropyl starch or carboxymethyl starch; polypeptides such as casein, gluten, gelatin, fibrin glue; chitosan, e.g. lactate or glutamate or carboxymethyl chitin; glycosaminoglycans such as hyaluronic acid; metals or water soluble salts of alginic acid such as sodium alginate or magnesium alginate; schleroglucan; adhesives containing bismuth oxide or aluminium oxide; atherocollagen; polyvinyl polymers such as carboxyvinyl polymers; polyvinylpyrrolidone (povidone); polyvinyl alcohol; polyvinyl acetates, polyvinylmethyl ethers, polyvinyl chlorides, polyvinylidenes, and/or the like; polycarboxylated vinyl polymers such as polyacrylic acid as mentioned above; polysiloxanes; polyethers; polyethylene oxides and glycols; polyalkoxys and polyacrylamides and derivatives and salts thereof. Preferred examples can include cellulose derivatives, such as methyl cellulose, ethyl cellulose, methylethyl cellulose, hydroxymethyl cellulose, hydroxyethyl cellulose, hydroxypropyl cellulose, hydroxyethyl ethyl cellulose, carboxymethyl cellulose, hydroxypropylmethyl cellulose or cellulose esters or ethers or derivatives or salts thereof (e.g., methyl cellulose); and polyvinyl polymers such as polyvinylpyrrolidone (povidone).

Examples of preservatives include without limitation: benzalkonium chloride, benzoxonium chloride, benzethonium chloride, cetrimide, sepazonium chloride, cetylpyridinium chloride, domiphen bromide (Bradosol®), thiomersal, phenylmercuric nitrate, phenylmercuric acetate, phenylmercuric borate, methylparaben, propylparaben, chlorobutanol, benzyl alcohol, phenyl ethyl alcohol, chlorohexidine, polyhexamethylene biguanide, sodium perborate, imidazolidinyl urea, sorbic acid, Purite®), Polyquart®), and sodium perborate tetrahydrate and the like.

In certain embodiments, the preservative is a paraben, or a pharmaceutically acceptable salt thereof. In some embodiments, the paraben is an alkyl substituted 4-hydroxybenzoate, or a pharmaceutically acceptable salt or ester thereof. In certain embodiments, the alkyl is a C₁-C₄ alkyl. In certain embodiments, the preservative is methyl 4-hydroxybenzoate (methylparaben), or a pharmaceutically acceptable salt or ester thereof, propyl 4-hydroxybenzoate (propylparaben), or a pharmaceutically acceptable salt or ester thereof, or a combination thereof.

Examples of buffers include without limitation: phosphate buffer system (sodium dihydrogen phospahate dehydrate, disodium phosphate dodecahydrate, bibasic sodium phosphate, anhydrous monobasic sodium phosphate), bicarbonate buffer system, and bisulfate buffer system.

Examples of disintegrants include, without limitation: carmellose calcium, low substituted hydroxypropyl cellulose (L-HPC), carmellose, croscarmellose sodium, partially pregelatinized starch, dry starch, carboxymethyl starch sodium, crospovidone, polysorbate 80 (polyoxyethylenesorbitan oleate), starch, sodium starch glycolate, hydroxypropyl cellulose pregelatinized starch, clays, cellulose, alginine, gums or cross linked polymers, such as crosslinked PVP (Polyplasdone XL from GAF Chemical Corp). In certain embodiments, the disintegrant is crospovidone.

Examples of glidants and lubricants (aggregation inhibitors) include without limitation: talc, magnesium stearate, calcium stearate, colloidal silica, stearic acid, aqueous silicon dioxide, synthetic magnesium silicate, fine granulated silicon oxide, starch, sodium laurylsulfate, boric acid, magnesium oxide, waxes, hydrogenated oil, polyethylene glycol, sodium benzoate, stearic acid glycerol behenate, polyethylene glycol, and mineral oil. In certain embodiments, the glidant/lubricant is magnesium stearate, talc, and/or colloidal silica; e.g., magnesium stearate and/or talc.

Examples of diluents, also referred to as “fillers” or “bulking agents” include without limitation: dicalcium phosphate dihydrate, calcium sulfate, lactose (e.g., lactose monohydrate), sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar. In certain embodiments, the diluent is lactose (e.g., lactose monohydrate).

Examples of binders include without limitation: starch, pregelatinized starch, gelatin, sugars (including sucrose, glucose, dextrose, lactose and sorbitol), polyethylene glycol, waxes, natural and synthetic gums such as acacia tragacanth, sodium alginate cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone (povidone). In certain embodiments, the binder is polyvinylpyrrolidone (povidone).

In some embodiments, enema formulations containing the chemical entities described herein include water and one or more (e.g., all) of the following excipients:
One or more (e.g., one, two, or three) thickeners, viscosity enhancing agents, binders, and/or mucoadhesive agents (e.g., cellulose or cellulose esters or ethers or derivatives or salts thereof (e.g., methyl cellulose); and polyvinyl polymers such as polyvinylpyrrolidone (povidone);
One or more (e.g., one or two; e.g., two) preservatives, such as a paraben, e.g., methyl 4-hydroxybenzoate (methylparaben), or a pharmaceutically acceptable salt or ester thereof, propyl 4-hydroxybenzoate (propylparaben), or a pharmaceutically acceptable salt or ester thereof, or a combination thereof;
One or more (e.g., one or two; e.g., two) buffers, such as phosphate buffer system (e.g., sodium dihydrogen phospahate dehydrate, disodium phosphate dodecahydrate);
One or more (e.g., one or two, e.g., two) glidants and/or lubricants, such as magnesium stearate and/or talc;
One or more (e.g., one or two; e.g., one) disintegrants, such as crospovidone; and
One or more (e.g., one or two; e.g., one) diluents, such as lactose (e.g., lactose monohydrate).

In certain of these embodiments, the chemical entity is a compound of Formula AA, or a pharmaceutically acceptable salt and/or hydrate and/or cocrystal thereof.

In certain embodiments, enema formulations containing the chemical entities described herein include water, methyl cellulose, povidone, methylparaben, propylparaben, sodium dihydrogen phospahate dehydrate, disodium phosphate dodecahydrate, crospovidone, lactose monohydrate, magnesium stearate, and talc. In certain of these embodiments, the chemical entity is a compound of Formula AA, or a pharmaceutically acceptable salt and/or hydrate and/or cocrystal thereof.

In certain embodiments, enema formulations containing the chemical entities described herein are provided in one or more kits or packs. In certain embodiments, the kit or pack includes two separately contained/packaged components, which when mixed together, provide the desired formulation (e.g., as a suspension). In certain of these embodiments, the two component system includes a first component and a second component, in which: (i) the first component (e.g., contained in a sachet) includes the chemical entity (as described anywhere herein) and one or more pharmaceutically acceptable excipients (e.g., together formulated as a solid preparation, e.g., together formulated as a wet granulated solid preparation); and (ii) the second component (e.g., contained in a vial or bottle) includes one or more liquids and one or more one or more other pharmaceutically acceptable excipients together forming a liquid carrier. In other embodiments, each of component (i) and (ii) is provided in its own separate kit or pack.

In certain of these embodiments, component (i) includes the chemical entity (e.g., a compound of Formula AA, or a pharmaceutically acceptable salt and/or hydrate and/or cocrystal thereof; e.g., a compound of Formula AA) and one or more (e.g., all) of the following excipients:
(a) One or more (e.g., one) binders (e.g., a polyvinyl polymer, such as polyvinylpyrrolidone (povidone);
(b) One or more (e.g., one or two, e.g., two) glidants and/or lubricants, such as magnesium stearate and/or talc;
(c) One or more (e.g., one or two; e.g., one) disintegrants, such as crospovidone; and
(d) One or more (e.g., one or two; e.g., one) diluents, such as lactose (e.g., lactose monohydrate).

In certain embodiments, component (i) includes from about 40 weight percent to about 80 weight percent (e.g., from about 50 weight percent to about 70 weight percent, from about 55 weight percent to about 70 weight percent; from about 60 weight percent to about 65 weight percent; e.g., about 62.1 weight percent) of the chemical entity (e.g., a compound of Formula AA, or a pharmaceutically acceptable salt and/or hydrate and/or cocrystal thereof).

In certain embodiments, component (i) includes from about 0.5 weight percent to about 5 weight percent (e.g., from about 1.5 weight percent to about 4.5 weight percent, from about 2 weight percent to about 3.5 weight percent; e.g., about 2.76 weight percent) of the binder (e.g., povidone).

In certain embodiments, component (i) includes from about 0.5 weight percent to about 5 weight percent (e.g., from about 0.5 weight percent to about 3 weight percent, from about 1 weight percent to about 3 weight percent; about 2 weight percent e.g., about 1.9 weight percent) of the disintegrant (e.g., crospovidone).

In certain embodiments, component (i) includes from about 10 weight percent to about 50 weight percent (e.g., from about 20 weight percent to about 40 weight percent, from about 25 weight percent to about 35 weight percent; e.g., about 31.03 weight percent) of the diluent (e.g., lactose, e.g., lactose monohydrate).

In certain embodiments, component (i) includes from about 0.05 weight percent to about 5 weight percent (e.g., from about 0.05 weight percent to about 3 weight percent) of the glidants and/or lubricants.

In certain embodiments (e.g., when component (i) includes one or more lubricants, such as magnesium stearate), component (i) includes from about 0.05 weight percent to about 1 weight percent (e.g., from about 0.05 weight percent to about 1 weight percent; from about 0.1 weight percent to about 1 weight percent; from about 0.1 weight percent to about 0.5 weight percent; e.g., about 0.27 weight percent) of the lubricant (e.g., magnesium stearate).

In certain embodiments (when component (i) includes one or more lubricants, such as talc), component (i) includes from about 0.5 weight percent to about 5 weight percent (e.g., from about 0.5 weight percent to about 3 weight percent, from about 1 weight percent to about 3 weight percent; from about 1.5 weight percent to about 2.5 weight percent; from about 1.8 weight percent to about 2.2 weight percent; about 1.93 weight percent) of the lubricant (e.g., talc).

In certain of these embodiments, each of (a), (b), (c), and (d) above is present.

In certain embodiments, component (i) includes the ingredients and amounts as shown in Table A.

TABLE A

| Ingredient | Weight Percent |
|---|---|
| A compound | 40 weight percent to about 80 weight percent (e.g., from |
| of Formula | about 50 weight percent to about 70 weight percent, from |
| AA | about 55 weight percent to about 70 weight percent; from |
| | about 60 weight percent to about 65 weight percent; e.g., |
| | about 62.1 weight percent) |
| Crospovidone | 0.5 weight percent to about 5 weight percent (e.g., from |
| (Kollidon CL) | about 0.5 weight percent to about 3 weight percent, from |
| | about 1 weight percent to about 3 weight percent; about |
| | 1.93 weight percent |
| lactose | about 10 weight percent to about 50 weight percent (e.g., |
| monohydrate | from about 20 weight percent to about 40 weight percent, |
| (Pharmatose | from about 25 weight percent to about 35 weight percent; |
| 200M) | e.g., about 31.03 weight percent |
| Povidone | about 0.5 weight percent to about 5 weight percent (e.g., |
| (Kollidon | from about 1.5 weight percent to about 4.5 weight |
| K30) | percent, from about 2 weight percent to about 3.5 weight |
| | percent; e.g., about 2.76 weight percent |
| talc | 0.5 weight percent to about 5 weight percent (e.g., from |
| | about 0.5 weight percent to about 3 weight percent, from |
| | about 1 weight percent to about 3 weight percent; from |
| | about 1.5 weight percent to about 2.5 weight percent; |
| | from about 1.8 weight percent to about 2.2 weight |
| | percent; e.g., about 1.93 weight percent |
| Magnesium | about 0.05 weight percent to about 1 weight percent |
| stearate | (e.g., from about 0.05 weight percent to about 1 weight |
| | percent; from about 0.1 weight percent to about 1 weight |
| | percent; from about 0.1 weight percent to about 0.5 |
| | weight percent; e.g., about 0.27 weight percent |

In certain embodiments, component (i) includes the ingredients and amounts as shown in Table B

TABLE B

| Ingredient | Weight Percent |
|---|---|
| A compound of Formula AA | About 62.1 weight percent) |
| Crospovidone (Kollidon CL) | About 1.93 weight percent |
| lactose monohydrate (Pharmatose 200M) | About 31.03 weight percent |
| Povidone (Kollidon K30) | About 2.76 weight percent |
| talc | About 1.93 weight percent |
| Magnesium stearate | About 0.27 weight percent |

In certain embodiments, component (i) is formulated as a wet granulated solid preparation. In certain of these embodiments an internal phase of ingredients (the chemical entity, disintegrant, and diluent) are combined and mixed in a high-shear granulator. A binder (e.g., povidone) is dissolved in water to form a granulating solution. This solution is added to the Inner Phase mixture resulting in the development of granules. While not wishing to be bound by theory, granule development is believed to be facilitated by the interaction of the polymeric binder with the materials of the internal phase. Once the granulation is formed and dried, an external phase (e.g., one or more lubricants—not an intrinsic component of the dried granulation), is added to the dry granulation. It is believed that lubrication of the granulation is important to the flowability of the granulation, in particular for packaging.

In certain of the foregoing embodiments, component (ii) includes water and one or more (e.g., all) of the following excipients:
(a′) One or more (e.g., one, two; e.g., two) thickeners, viscosity enhancing agents, binders, and/or mucoadhesive agents (e.g., cellulose or cellulose esters or ethers or derivatives or salts thereof (e.g., methyl cellulose); and polyvinyl polymers such as polyvinylpyrrolidone (povidone);
(b′) One or more (e.g., one or two; e.g., two) preservatives, such as a paraben, e.g., methyl 4-hydroxybenzoate (methylparaben), or a pharmaceutically acceptable salt or ester thereof, propyl 4-hydroxybenzoate (propylparaben), or a pharmaceutically acceptable salt or ester thereof, or a combination thereof; and
(c′) One or more (e.g., one or two; e.g., two) buffers, such as phosphate buffer system (e.g., sodium dihydrogen phospahate dihydrate, disodium phosphate dodecahydrate);

In certain of the foregoing embodiments, component (ii) includes water and one or more (e.g., all) of the following excipients:
(a″) a first thickener, viscosity enhancing agent, binder, and/or mucoadhesive agent (e.g., a cellulose or cellulose ester or ether or derivative or salt thereof (e.g., methyl cellulose));
(a′″) a second thickener, viscosity enhancing agent, binder, and/or mucoadhesive agent (e.g., a polyvinyl polymer, such as polyvinylpyrrolidone (povidone));
(b″) a first preservative, such as a paraben, e.g., propyl 4-hydroxybenzoate (propylparaben), or a pharmaceutically acceptable salt or ester thereof;
(b″) a second preservative, such as a paraben, e.g., methyl 4-hydroxybenzoate (methylparaben), or a pharmaceutically acceptable salt or ester thereof,
(c″) a first buffer, such as phosphate buffer system (e.g., disodium phosphate dodecahydrate);
(c′″) a second buffer, such as phosphate buffer system (e.g., sodium dihydrogen phospahate dehydrate),

In certain embodiments, component (ii) includes from about 0.05 weight percent to about 5 weight percent (e.g., from about 0.05 weight percent to about 3 weight percent, from about 0.1 weight percent to about 3 weight percent; e.g., about 1.4 weight percent) of (a″).

In certain embodiments, component (ii) includes from about 0.05 weight percent to about 5 weight percent (e.g., from about 0.05 weight percent to about 3 weight percent, from about 0.1 weight percent to about 2 weight percent; e.g., about 1.0 weight percent) of (a′″).

In certain embodiments, component (ii) includes from about 0.005 weight percent to about 0.1 weight percent (e.g., from about 0.005 weight percent to about 0.05 weight percent; e.g., about 0.02 weight percent) of (b″).

In certain embodiments, component (ii) includes from about 0.05 weight percent to about 1 weight percent (e.g., from about 0.05 weight percent to about 0.5 weight percent; e.g., about 0.20 weight percent) of (b′″).

In certain embodiments, component (ii) includes from about 0.05 weight percent to about 1 weight percent (e.g., from about 0.05 weight percent to about 0.5 weight percent; e.g., about 0.15 weight percent) of (c″).

In certain embodiments, component (ii) includes from about 0.005 weight percent to about 0.5 weight percent (e.g., from about 0.005 weight percent to about 0.3 weight percent; e.g., about 0.15 weight percent) of (c′″).

In certain of these embodiments, each of (a″)-(c′″) is present.

In certain embodiments, component (ii) includes water (up to 100%) and the ingredients and amounts as shown in Table C.

TABLE C

| Ingredient | Weight Percent |
|---|---|
| methyl cellulose | 0.05 weight percent to about 5 weight percent |
| (Methocel A15C | (e.g., from about 0.05 weight percent to about |
| premium) | 3 weight percent, from about 0.1 weight percent |
| | to about 3 weight percent; e.g., about 1.4 |
| | weight percent |
| Povidone | 0.05 weight percent to about 5 weight percent |
| (Kollidon K30) | (e.g., from about 0.05 weight percent to about |
| | 3 weight percent, from about 0.1 weight percent |
| | to about 2 weight percent; e.g., about 1.0 weight |
| | percent |
| propyl | about 0.005 weight percent to about 0.1 weight |
| 4-hydroxybenzoate | percent (e.g., from about 0.005 weight percent |
| | to about 0.05 weight percent; e.g., about 0.02 |
| | weight percent) |
| methyl | about 0.05 weight percent to about 1 weight percent |
| 4-hydroxybenzoate | (e.g., from about 0.05 weight percent to about 0.5 |
| | weight percent; e.g., about 0.20 weight percent) |
| disodium phosphate | about 0.05 weight percent to about 1 weight percent |
| dodecahydrate | (e.g., from about 0.05 weight percent to about 0.5 |
| | weight percent; e.g., about 0.15 weight percent) |
| sodium dihydrogen | about 0.005 weight percent to about 0.5 weight |
| phospahate | percent (e.g., from about 0.005 weight percent |
| dihydrate | to about 0.3 weight percent; e.g., about 0.15 |
| | weight percent) |

In certain embodiments, component (ii) includes water (up to 100%) and the ingredients and amounts as shown in Table D.

TABLE D

| Ingredient | Weight Percent |
|---|---|
| methyl cellulose (Methocel A15C premium) | about 1.4 weight percent |
| Povidone (Kollidon K30) | about 1.0 weight percent |
| propyl 4-hydroxybenzoate | about 0.02 weight percent |
| methyl 4-hydroxybenzoate | about 0.20 weight percent |
| disodium phosphate dodecahydrate | about 0.15 weight percent |
| sodium dihydrogen phospahate dihydrate | about 0.15 weight percent |

Ready-to-use” enemas are generally be provided in a “single-use” sealed disposable container of plastic or glass. Those formed of a polymeric material preferably have sufficient flexibility for ease of use by an unassisted patient. Typical plastic containers can be made of polyethylene. These containers may comprise a tip for direct introduction into the rectum. Such containers may also comprise a tube between the container and the tip. The tip is preferably provided with a protective shield which is removed before use. Optionally the tip has a lubricant to improve patient compliance.

In some embodiments, the enema formulation (e.g., suspension) is poured into a bottle for delivery after it has been prepared in a separate container. In certain embodiments, the bottle is a plastic bottle (e.g., flexible to allow for delivery by squeezing the bottle), which can be a polyethylene bottle (e.g., white in color). In some embodiments, the bottle is a single chamber bottle, which contains the suspension or solution. In other embodiments, the bottle is a multichamber bottle, where each chamber contains a separate mixture or solution. In still other embodiments, the bottle can further include a tip or rectal cannula for direct introduction into the rectum. In some embodiments, the enema formulation can be delivered in a device such as, a plastic bottle, a breakable capsule, and a rectal cannula and single flow pack.

Dosages

The dosages may be varied depending on the requirement of the patient, the severity of the condition being treating and the particular compound being employed. Determination of the proper dosage for a particular situation can be determined by one skilled in the medical arts. The total daily dosage may be divided and administered in portions throughout the day or by means providing continuous delivery.

In some embodiments, the compounds described herein are administered at a dosage of from about 0.001 mg/Kg to about 500 mg/Kg (e.g., from about 0.001 mg/Kg to about 200 mg/Kg; from about 0.01 mg/Kg to about 200 mg/Kg; from about 0.01 mg/Kg to about 150 mg/Kg; from about 0.01 mg/Kg to about 100 mg/Kg; from about 0.01 mg/Kg to about 50 mg/Kg; from about 0.01 mg/Kg to about 10 mg/Kg; from about 0.01 mg/Kg to about 5 mg/Kg; from about 0.01 mg/Kg to about 1 mg/Kg; from about 0.01 mg/Kg to about 0.5 mg/Kg; from about 0.01 mg/Kg to about 0.1 mg/Kg; from about 0.1 mg/Kg to about 200 mg/Kg; from about 0.1 mg/Kg to about 150 mg/Kg; from about 0.1 mg/Kg to about 100 mg/Kg; from about 0.1 mg/Kg to about 50 mg/Kg; from about 0.1 mg/Kg to about 10 mg/Kg; from about 0.1 mg/Kg to about 5 mg/Kg; from about 0.1 mg/Kg to about 1 mg/Kg; from about 0.1 mg/Kg to about 0.5 mg/Kg).

In some embodiments, enema formulations include from about 0.5 mg to about 2500 mg (e.g., from about 0.5 mg to about 2000 mg, from about 0.5 mg to about 1000 mg, from about 0.5 mg to about 750 mg, from about 0.5 mg to about 600 mg, from about 0.5 mg to about 500 mg, from about 0.5 mg to about 400 mg, from about 0.5 mg to about 300 mg, from about 0.5 mg to about 200 mg; e.g., from about 5 mg to about 2500 mg, from about 5 mg to about 2000 mg, from about 5 mg to about 1000 mg; from about 5 mg to about 750 mg; from about 5 mg to about 600 mg; from about 5 mg to about 500 mg; from about 5 mg to about 400 mg; from about 5 mg to about 300 mg; from about 5 mg to about 200 mg; e.g., from about 50 mg to about 2000 mg, from about 50 mg to about 1000 mg, from about 50 mg to about 750 mg, from about 50 mg to about 600 mg, from about 50 mg to about 500 mg, from about 50 mg to about 400 mg, from about 50 mg to about 300 mg, from about 50 mg to about 200 mg; e.g., from about 100 mg to about 2500 mg, from about 100 mg to about 2000 mg, from about 100 mg to about 1000 mg, from about 100 mg to about 750 mg, from about 100 mg to about 700 mg, from about 100 mg to about 600 mg, from about 100 mg to about 500 mg, from about 100 mg to about 400 mg, from about 100 mg to about 300 mg, from about 100 mg to about 200 mg; e.g., from about 150 mg to about 2500 mg, from about 150 mg to about 2000 mg, from about 150 mg to about 1000 mg, from about 150 mg to about 750 mg, from about 150 mg to about 700 mg, from about 150 mg to about 600 mg, from about 150 mg to about 500 mg, from about 150 mg to about 400 mg, from about 150 mg to about 300 mg, from about 150 mg to about 200 mg; e.g., from about 150 mg to about 500 mg; e.g., from about 300 mg to about 2500 mg, from about 300 mg to about 2000 mg, from about 300 mg to about 1000 mg, from about 300 mg to about 750 mg, from about 300 mg to about 700 mg, from about 300 mg to about 600 mg; e.g., from about 400 mg to about 2500 mg, from about 400 mg to about 2000 mg, from about 400 mg to about 1000 mg, from about 400 mg to about 750 mg, from about 400 mg to about 700 mg, from about 400 mg to about 600 from about 400 mg to about 500 mg; e.g., 150 mg or 450 mg) of the chemical entity in from about 1 mL to about 3000 mL (e.g., from about 1 mL to about 2000 mL, from about 1 mL to about 1000 mL, from about 1 mL to about 500 mL, from about 1 mL to about 250 mL, from about 1 mL to about 100 mL, from about 10 mL to about 1000 mL, from about 10 mL to about 500 mL, from about 10 mL to about 250 mL, from about 10 mL to about 100 mL, from about 30 mL to about 90 mL, from about 40 mL to about 80 mL; from about 50 mL to about 70 mL; e.g., about 1 mL, about 5 mL, about 10 mL, about 15 mL, about 20 mL, about 25 mL, about 30 mL, about 35 mL, about 40 mL, about 45 mL, about 50 mL, about 55 mL, about 60 mL, about 65 mL, about 70 mL, about 75 mL, about 100 mL, about 250 mL, or about 500 mL, or about 1000 mL, or about 2000 mL, or about 3000 mL; e.g., 60 mL) of liquid carrier.

In certain embodiments, enema formulations include from about 50 mg to about 250 mg (e.g., from about 100 mg to about 200; e.g., about 150 mg) of the chemical entity in from about 10 mL to about 100 mL (e.g., from about 20 mL to about 100 mL, from about 30 mL to about 90 mL, from about 40 mL to about 80 mL; from about 50 mL to about 70 mL) of liquid carrier. In certain embodiments, enema formulations include about 150 mg of the chemical entity in about 60 mL of the liquid carrier. In certain of these embodiments, the chemical entity is a compound of Formula AA, or a pharmaceutically acceptable salt and/or hydrate and/or cocrystal thereof. For example, enema formulations can include about 150 mg of a compound of Formula AA in about 60 mL of the liquid carrier.

In certain embodiments, enema formulations include from about 350 mg to about 550 mg (e.g., from about 400 mg to about 500; e.g., about 450 mg) of the chemical entity in from about 10 mL to about 100 mL (e.g., from about 20 mL to about 100 mL, from about 30 mL to about 90 mL, from about 40 mL to about 80 mL; from about 50 mL to about 70 mL) of liquid carrier. In certain embodiments, enema formulations include about 450 mg of the chemical entity in about 60 mL of the liquid carrier. In certain of these embodiments, the chemical entity is a compound of Formula AA, or a pharmaceutically acceptable salt and/or hydrate and/or cocrystal thereof. For example, enema formulations can include about 450 mg of a compound of Formula AA in about 60 mL of the liquid carrier.

In some embodiments, enema formulations include from about from about 0.01 mg/mL to about 50 mg/mL (e.g., from about 0.01 mg/mL to about 25 mg/mL; from about 0.01 mg/mL to about 10 mg/mL; from about 0.01 mg/mL to about 5 mg/mL; from about 0.1 mg/mL to about 50 mg/mL; from about 0.01 mg/mL to about 25 mg/mL; from about 0.1 mg/mL to about 10 mg/mL; from about 0.1 mg/mL to about 5 mg/mL; from about 1 mg/mL to about 10 mg/mL; from about 1 mg/mL to about 5 mg/mL; from about 5 mg/mL to about 10 mg/mL; e.g., about 2.5 mg/mL or about 7.5 mg/mL) of the chemical entity in liquid carrier. In certain of these embodiments, the chemical entity is a compound of Formula AA, or a pharmaceutically acceptable salt and/or hydrate and/or cocrystal thereof. For example, enema formulations can include about 2.5 mg/mL or about 7.5 mg/mL of a compound of Formula AA in liquid carrier.

Regimens

The foregoing dosages can be administered on a daily basis (e.g., as a single dose or as two or more divided doses) or non-daily basis (e.g., every other day, every two days, every three days, once weekly, twice weeks, once every two weeks, once a month).

In some embodiments, the period of administration of a compound described herein is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In an embodiment, a therapeutic compound is administered to an individual for a period of time followed by a separate period of time. In another embodiment, a therapeutic compound is administered for a first period and a second period following the first period, with administration stopped during the second period, followed by a third period where administration of the therapeutic compound is started and then a fourth period following the third period where administration is stopped. In an aspect of this embodiment, the period of administration of a therapeutic compound followed by a period where administration is stopped is repeated for a determined or undetermined period of time. In a further embodiment, a period of administration is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

Methods of Treatment

In some embodiments, methods for treating a subject having condition, disease or disorder in which a decrease or increase in NLRP3 activity (e.g., an increase, e.g., NLRP3 signaling) contributes to the pathology and/or symptoms and/or progression of the condition, disease or disorder are provided, comprising administering to a subject an effective amount of a chemical entity described herein (e.g., a compound described generically or specifically herein or a pharmaceutically acceptable salt thereof or compositions containing the same).

Indications

In some embodiments, the condition, disease or disorder is selected from: inappropriate host responses to infectious diseases where active infection exists at any body site, such as septic shock, disseminated intravascular coagulation, and/or adult respiratory distress syndrome; acute or chronic inflammation due to antigen, antibody and/or complement deposition; inflammatory conditions including arthritis, cholangitis, colitis, encephalitis, endocarditis, glomerulonephritis, hepatitis, myocarditis, pancreatitis, pericarditis, reperfusion injury and vasculitis, immune-based diseases such as acute and delayed hypersensitivity, graft rejection, and graft-versus-host disease; auto-immune diseases including Type 1 diabetes mellitus and multiple sclerosis. For example, the condition, disease or disorder may be an inflammatory disorder such as rheumatoid arthritis, osteoarthritis, septic shock, COPD and periodontal disease.

In some embodiments, the condition, disease or disorder is an autoimmune diseases. Non-limiting examples include rheumatoid arthritis, systemic lupus erythematosus, multiple sclerosis, inflammatory bowel diseases (IBDs) comprising Crohn disease (CD) and ulcerative colitis (UC), which are chronic inflammatory conditions with polygenic susceptibility. In certain embodiments, the condition is an inflammatory bowel disease. In certain embodiments, the condition is Crohn's disease, autoimmune colitis, iatrogenic autoimmune colitis, ulcerative colitis, colitis induced by one or more chemotherapeutic agents, colitis induced by treatment with adoptive cell therapy, colitis associated by one or more alloimmune diseases (such as graft-vs-host disease, e.g., acute graft vs. host disease and chronic graft vs. host disease), radiation enteritis, collagenous colitis, lymphocytic colitis, microscopic colitis, and radiation enteritis. In certain of these embodiments, the condition is alloimmune disease (such as graft-vs-host disease, e.g., acute graft vs. host disease and chronic graft vs. host disease), celiac disease, irritable bowel syndrome, rheumatoid arthritis, lupus, scleroderma, psoriasis, cutaneous T-cell lymphoma, uveitis, and mucositis (e.g., oral mucositis, esophageal mucositis or intestinal mucositis).

Without being bound by theory, it is believed that the presence of the two substituents R^{1a} and R^{1b} as defined herein result in compounds that cross the intestinal barrier in a limited manner and are therefore result in compounds that are restricted to the gut and provide targeted delivery to the gut. Applicants have surprisingly found that the presence of at least two substituents, and particularly two polar substituents R^{1a} and R^{1b} may provide compounds of formula AA that are poorly absorbed into systemic circulation after oral administration and are therefore restricted to the gut. Without being bound by theory, it is further hypothesized that the gut restricted compounds of the present invention may be used for treatment or prevention or alleviation of symptoms of certain gastrointestinal disorders. It is also hypothesized that the targeting of compounds to the gut may reduce the incidence of side effects due to systemic absorption of compounds.

In some embodiments, the condition, disease or disorder is selected from major adverse cardiovascular events such as carbiovascular death, non-fatal myocardial infarction and non-fatal stroke in patients with a prior hear attack and inflammatory atherosclerosis (see for example, NCT01327846).

In some embodiments, the condition, disease or disorder is selected from metabolic disorders such as type 2 diabetes, atherosclerosis, obesity and gout, as well as diseases of the central nervous system, such as Alzheimer's disease and multiple sclerosis and Amyotrophic Lateral Sclerosis and Parkinson disease, lung disease, such as asthma and COPD and pulmonary idiopathic fibrosis, liver disease, such as NASH syndrome, viral hepatitis and cirrhosis, pancreatic disease, such as acute and chronic pancreatitis, kidney disease, such as acute and chronic kidney injury, intestinal disease such as Crohn's disease and Ulcerative Colitis, skin disease such as psoriasis, musculoskeletal disease such as scleroderma, vessel disorders, such as giant cell arteritis, disorders of the bones, such as Osteoarthritis, osteoporosis and osteopetrosis disorders eye disease, such as glaucoma and macular degeneration, diseased caused by viral infection such as HIV and AIDS, autoimmune disease such as Rheumatoid Arthritis, Systemic Lupus Erythematosus, Autoimmune Thyroiditis, Addison's disease, pernicious anemia, cancer and aging.

In some embodiments, the condition, disease or disorder is a cardiovascular indication. In some embodiments, the condition, disease or disorder is myocardial infraction. In some embodiments, the condition, disease or disorder is stroke.

In some embodiments, the condition, disease or disorder is obesity. In some embodiments, the condition, disease or disorder is Type 2 Diabetes. In some embodiments, the condition, disease or disorder is NASH. In some embodiments, the condition, disease or disorder is Alzheimer's disease. In some embodiments, the condition, disease or disorder is gout. In some embodiments, the condition, disease or disorder is SLE. In some embodiments, the condition, disease or disorder is rheumatoid arthritis. In some embodiments, the condition, disease or disorder is IBD. In some embodiments, the condition, disease or disorder is multiple sclerosis. In some embodiments, the condition, disease or disorder is COPD. In some embodiments, the condition, disease or disorder is asthma. In some embodiments, the condition, disease or disorder is scleroderma. In some embodiments, the condition, disease or disorder is pulmonary fibrosis. In some embodiments, the condition, disease or disorder is age related macular degeneration (AMD). In some embodiments, the condition, disease or disorder is cystic fibrosis. In some embodiments, the condition, disease or disorder is Muckle Wells syndrome. In some embodiments, the condition, disease or disorder is familial cold autoinflammatory syndrome (FCAS). In some embodiments, the condition, disease or disorder is chronic neurologic cutaneous and articular syndrome.

In some embodiments, the condition, disease or disorder is selected from: myelodysplastic syndromes (MDS); non-small cell lung cancer, such as non-small cell lung cancer in patients carrying mutation or overexpression of NLRP3; acute lymphoblastic leukemia (ALL), such as ALL in patients resistant to glucocorticoids treatment; Langerhan's cell histiocytosis (LCH); multiple myeloma; promyelocytic leukemia; acute myeloid leukemia (AML) chronic myeloid leukemia (CML); gastric cancer; and lung cancer metastasis.

In some embodiments, the condition, disease or disorder is selected from: myelodysplastic syndromes (MDS); non-small cell lung cancer, such as non-small cell lung cancer in patients carrying mutation or overexpression of NLRP3; acute lymphoblastic leukemia (ALL), such as ALL in patients resistant to glucocorticoids treatment; Langerhan's cell histiocytosis (LCH); multiple myeloma; promyelocytic leukemia; gastric cancer; and lung cancer metastasis.

In some embodiments, the indication is MDS. In some embodiments, the indication is non-small lung cancer in patients carrying mutation or overexpression of NLRP3.

In some embodiments, the indication is ALL in patients resistant to glucocorticoids treatment.

In some embodiments, the indication is LCH. In some embodiments, the indication is multiple myeloma. In some embodiments, the indication is promyelocytic leukemia. In some embodiments, the indication is gastric cancer. In some embodiments, the indication is lung cancer metastasis.

Combination Therapy

This disclosure contemplates both monotherapy regimens as well as combination therapy regimens.

In some embodiments, the methods described herein can further include administering one or more additional therapies (e.g., one or more additional therapeutic agents and/or one or more therapeutic regimens) in combination with administration of the compounds described herein.

In certain embodiments, the second therapeutic agent or regimen is administered to the subject prior to contacting with or administering the chemical entity (e.g., about one hour prior, or about 6 hours prior, or about 12 hours prior, or about 24 hours prior, or about 48 hours prior, or about 1 week prior, or about 1 month prior).

In other embodiments, the second therapeutic agent or regimen is administered to the subject at about the same time as contacting with or administering the chemical entity. By way of example, the second therapeutic agent or regimen and the chemical entity are provided to the subject simultaneously in the same dosage form. As another example, the second therapeutic agent or regimen and the chemical entity are provided to the subject concurrently in separate dosage forms.

In still other embodiments, the second therapeutic agent or regimen is administered to the subject after contacting with or administering the chemical entity (e.g., about one hour after, or about 6 hours after, or about 12 hours after, or about 24 hours after, or about 48 hours after, or about 1 week after, or about 1 month after).

Patient Selection

In some embodiments, the methods described herein further include the step of identifying a subject (e.g., a patient) in need of treatment for an indication related to NLRP3 activity, such as an indication related to NLRP3 polymorphism.

In some embodiments, the methods described herein further include the step of identifying a subject (e.g., a patient) in need of treatment for an indication related to NLRP3 activity, such as an indication related to NLRP3 where polymorphism is a gain of function

In some embodiments, the methods described herein further include the step of identifying a subject (e.g., a patient) in need of treatment for an indication related to NLRP3 activity, such as an indication related to NLRP3 polymorphism found in CAPS syndromes.

In some embodiments, the methods described herein further include the step of identifying a subject (e.g., a patient) in need of treatment for an indication related to NLRP3 activity, such as an indication related NLRP3 polymorphism where the polymorphism is VAR_014104 (R262W)

In some embodiments, the methods described herein further include the step of identifying a subject (e.g., a patient) in need of treatment for an indication related to NLRP3 activity, such as an indication related NLRP3 polymorphism where the polymorphism is a natural variant reported in http://www.uniprot.org/uniprot/Q96P20.

In some embodiments, the methods described herein further include the step of identifying a subject (e.g., a patient) in need of treatment for an indication related to NLRP3 activity, such as an indication related to point mutation of NLRP3 signaling.

### Anti-TNFα Agents

The term “anti-TNFα agent” refers to an agent which directly or indirectly blocks, down-regulates, impairs, inhibits, impairs, or reduces TNFα activity and/or expression. In some embodiments, an anti-TNFα agent is an antibody or an antigen-binding fragment thereof, a fusion protein, a soluble TNFα receptor (a soluble tumor necrosis factor receptor superfamily member 1A (TNFR1) or a soluble tumor necrosis factor receptor superfamily IB (TNFR2)), an inhibitory nucleic acid, or a small molecule TNFα antagonist. In some embodiments, the inhibitory nucleic acid is a ribozyme, small hairpin RNA, a small interfering RNA, an antisense nucleic acid, or an aptamer.

Exemplary anti-TNFα agents that directly block, down-regulate, impair, inhibit, or reduce TNFα activity and/or expression can, e.g., inhibit or decrease the expression level of TNFα or a receptor of TNFα (TNFR1 or TNFR2) in a cell (e.g., a cell obtained from a subject, a mammalian cell), or inhibit or reduce binding of TNFα to its receptor (TNFR1 and/or TNFR2) and/or. Non-limiting examples of anti-TNFα agents that directly block, down-regulate, impair, inhibit, or reduce TNFα activity and/or expression include an antibody or fragment thereof, a fusion protein, a soluble TNFα receptor (e.g., a soluble TNFR1 or soluble TNFR2), inhibitory nucleic acids (e.g., any of the examples of inhibitory nucleic acids described herein), and a small molecule TNFα antagonist.

Exemplary anti-TNFα agents that can indirectly block, down-regulate, impair, inhibitreduce TNFα activity and/or expression can, e.g., inhibit or decrease the level of downstream signaling of a TNFα receptor (e.g., TNFR1 or TNFR2) in a mammalian cell (e.g., decrease the level and/or activity of one or more of the following signaling proteins: AP-1, mitogen-activated protein kinase kinase kinase 5 (ASK1), inhibitor of nuclear factor kappa B (IKK), mitogen-activated protein kinase 8 (INK), mitogen-activated protein kinase (MAPK), MEKK 1/4, MEKK 4/7, MEKK 3/6, nuclear factor kappa B (NF-κB), mitogen-activated protein kinase kinase kinase 14 (NIK), receptor interacting serine/threonine kinase 1 (RIP), TNFRSF1A associated via death domain (TRADD), and TNF receptor associated factor 2 (TRAF2), in a cell), and/or decrease the level of TNFα-induced gene expression in a mammalian cell (e.g., decrease the transcription of genes regulated by, e.g., one or more transcription factors selected from the group of activating transcription factor 2 (ATF2), c-Jun, and NF-κB). A description of downstream signaling of a TNFα receptor is provided in Wajant et al., Cell Death Differentiation 10:45-65, 2003 (incorporated herein by reference). For example, such indirect anti-TNFα agents can be an inhibitory nucleic acid that targets (decreases the expression) a signaling component downstream of a TNFα-induced gene (e.g., any TNFα-induced gene known in the art), a TNFα receptor (e.g., any one or more of the signaling components downstream of a TNFα receptor described herein or known in the art), or a transcription factor selected from the group of NF-κB, c-Jun, and ATF2.

In other examples, such indirect anti-TNFα agents can be a small molecule inhibitor of a protein encoded by a TNFα-induced gene (e.g., any protein encoded by a TNFα-induced gene known in the art), a small molecule inhibitor of a signaling component downstream of a TNFα receptor (e.g., any of the signaling components downstream of a TNFα receptor described herein or known in the art), and a small molecule inhibitor of a transcription factor selected from the group of ATF2, c-Jun, and NF-κB.

In other embodiments, anti-TNFα agents that can indirectly block, down-regulate, impair, or reduce one or more components in a cell (e.g., a cell obtained from a subject, a mammalian cell) that are involved in the signaling pathway that results in TNFα mRNA transcription, TNFα mRNA stabilization, and TNFα mRNA translation (e.g., one or more components selected from the group of CD14, c-Jun, ERK1/2, IKK, IκB, interleukin 1 receptor associated kinase 1 (IRAK), JNK, lipopolysaccharide binding protein (LBP), MEK1/2, MEK3/6, MEK4/7, MK2, MyD88, NF-κB, NIK, PKR, p38, AKT serine/threonine kinase 1 (rac), raf kinase (raf), ras, TRAF6, TTP). For example, such indirect anti-TNFα agents can be an inhibitory nucleic acid that targets (decreases the expression) of a component in a mammalian cell that is involved in the signaling pathway that results in TNFα mRNA transcription, TNFα mRNA stabilization, and TNFα mRNA translation (e.g., a component selected from the group of CD14, c-Jun, ERK1/2, IKK, IκB, IRAK, JNK, LBP, MEK1/2, MEK3/6, MEK4/7, MK2, MyD88, NF-κB, NIK, IRAK, lipopolysaccharide binding protein (LBP), PKR, p38, rac, raf, ras, TRAF6, TTP). In other examples, an indirect anti-TNFα agents is a small molecule inhibitor of a component in a mammalian cell that is involved in the signaling pathway that results in TNFα mRNA transcription, TNFα mRNA stabilization, and TNFα mRNA translation (e.g., a component selected from the group of CD14, c-Jun, ERK1/2, IKK, IκB, IRAK, JNK, lipopolysaccharide binding protein (LBP), MEK1/2, MEK3/6, MEK4/7, MK2, MyD88, NF-κB, NIK, IRAK, lipopolysaccharide binding protein (LBP), PKR, p38, rac, raf, ras, TRAF6, TTP).

### Antibodies

In some embodiments, the anti-TNFα agent is an antibody or an antigen-binding fragment thereof (e.g., a Fab or a scFv). In some embodiments, an antibody or antigen-binding fragment of an antibody described herein can bind specifically to TNFα. In some embodiments, an antibody or antigen-binding fragment described herein binds specifically to any one of TNFα, TNFR1, or TNFR2. In some embodiments, an antibody or antigen-binding fragment of an antibody described herein can bind specifically to a TNFα receptor (TNFR1 or TNFR2).

In some embodiments, the antibody can be a humanized antibody, a chimeric antibody, a multivalent antibody, or a fragment thereof. In some embodiments, an antibody can be a scFv-Fc, a VHH domain, a VNAR domain, a (scFv)2, a minibody, or a BiTE.

In some embodiments, an antibody can be a crossmab, a diabody, a scDiabody, a scDiabody-CH3, a Diabody-CH3, a DutaMab, a DT-IgG, a diabody-Fc, a scDiabody-HAS, a charge pair antibody, a Fab-arm exchange antibody, a SEEDbody, a Triomab, a LUZ-Y, a Fcab, a kX-body, an orthogonal Fab, a DVD-IgG, an IgG(H)-scFv, a scFv-(H)IgG, an IgG(L)-scFv, a scFv-(L)-IgG, an IgG (L,H)-Fc, an IgG(H)-V, a V(H)-IgG, an IgG(L)-V, a V(L)-IgG, an KIH IgG-scFab, a 2scFv-IgG, an IgG-2scFv, a scFv4-Ig, a Zybody, a DVI-IgG, a nanobody, a nanobody-HSA, a DVD-Ig, a dual-affinity re-targeting antibody (DART), a triomab, a kih IgG with a common LC, an ortho-Fab IgG, a 2-in-1-IgG, IgG-ScFv, scFv2-Fc, a bi-nanobody, tanden antibody, a DART-Fc, a scFv-HAS-scFv, a DAF (two-in-one or four-in-one), a DNL-Fab3, knobs-in-holes common LC, knobs-in-holes assembly, a TandAb, a Triple Body, a miniantibody, a minibody, a TriBi minibody, a scFv-CH3 KIH, a Fab-scFv, a scFv-CH-CL-scFv, a F(ab′)2-scFV2, a scFv-KIH, a Fab-scFv-Fc, a tetravalent HCAb, a scDiabody-Fc, a tandem scFv-Fc, an intrabody, a dock and lock bispecific antibody, an ImmTAC, a HSAbody, a tandem scFv, an IgG-IgG, a Cov-X-Body, and a scFv1-PEG-scFv2.

Non-limiting examples of an antigen-binding fragment of an antibody include an Fv fragment, a Fab fragment, a F(ab′)2 fragment, and a Fab′ fragment. Additional examples of an antigen-binding fragment of an antibody is an antigen-binding fragment of an antigen-binding fragment of an IgA (e.g., an antigen-binding fragment of IgA1 or IgA2) (e.g., an antigen-binding fragment of a human or humanized IgA, e.g., a human or humanized IgA1 or IgA2); an antigen-binding fragment of an IgD (e.g., an antigen-binding fragment of a human or humanized IgD); an antigen-binding fragment of an IgE (e.g., an antigen-binding fragment of a human or humanized IgE); an IgG (e.g., an antigen-binding fragment of IgG1, IgG2, IgG3, or IgG4) (e.g., an antigen-binding fragment of a human or humanized IgG, e.g., human or humanized IgG1, IgG2, IgG3, or IgG4); or an antigen-binding fragment of an IgM (e.g., an antigen-binding fragment of a human or humanized IgM).

Non-limiting examples of anti-TNFα agents that are antibodies that specifically bind to TNFα are described in Ben-Horin et al., Autoimmunity Rev. 13(1):24-30, 2014; Bongartz et al., JAMA 295(19):2275-2285, 2006; Butler et al., Eur. Cytokine Network 6(4):225-230, 1994; Cohen et al., Canadian J. Gastroenterol. Hepatol. 15(6):376-384, 2001; Elliott et al., Lancet 1994; 344: 1125-1127, 1994; Feldmann et al., Ann. Rev. Immunol. 19(1): 163-196, 2001; Rankin et al., Br. J. Rheumatol. 2:334-342, 1995; Knight et al., Molecular Immunol. 30(16): 1443-1453, 1993; Lorenz et al., J. Immunol. 156(4): 1646-1653, 1996; Hinshaw et al., Circulatory Shock 30(3):279-292, 1990; Ordas et al., Clin. Pharmacol. Therapeutics 91(4):635-646, 2012; Feldman, Nature Reviews Immunol. 2(5):364-371, 2002; Taylor et al., Nature Reviews Rheumatol. 5(10):578-582, 2009; Garces et al., Annals Rheumatic l)is. 72(12): 1947-1955, 2013; Palladino et al., Nature Rev. Drug Discovery 2(9):736-746, 2003; Sandbom et al., Inflammatory Bowel Diseases 5(2): 119-133, 1999; Atzeni et al., Autoimmunity Reviews 12(7):703-708, 2013; Maini et al., Immunol. Rev. 144(1): 195-223, 1995; Wanner et al., Shock 11(6):391-395, 1999; and U.S. Pat. Nos. 6,090,382; 6,258,562; and 6,509,015).

In certain embodiments, the anti-TNFα agent can include or is golimumab (Golimumab™), adalimumab (Humira™), infliximab (Remicade™), CDP571, CDP 870, or certolizumab pegol (Cimzia™). In certain embodiments, the anti-TNFα agent can be a TNFα inhibitor biosimilar. Examples of approved and late-phase TNFα inhibitor biosimilars include, but are not limited to, infliximab biosimilars such as Flixabi™ (SB2) from Samsung Bioepis, Inflectra® (CT-P13) from Celltrion/Pflzer, GS071 from Aprogen, Remsima™, PF-06438179 from Pfizer/Sandoz, NI-071 from Nichi-Iko Pharmaceutical Co., and ABP 710 from Amgen; adalimumab biosimilars such as Amgevita® (ABP 501) from Amgen and Exemptia™ from Zydus Cadila, BMO-2 or MYL-1401-A from Biocon/Mylan, CHS-1420 from Coherus, FKB327 from Kyowa Kirin, and BI 695501 from Boehringer Ingelheim; Solymbic®, SB5 from Samsung Bioepis, GP-2017 from Sandoz, ONS-3010 from Oncobiologics, M923 from Momenta, PF-06410293 from Pfizer, and etanercept biosimilars such as Erelzi™ from Sandoz/Novartis, Brenzys™ (SB4) from Samsung Bioepis, GP2015 from Sandoz, TuNEX® from Mycenax, LBEC0101 from LG Life, and CHS-0214 from Coherus.

In some embodiments of any of the methods described herein, the anti-TNFα agent is selected from the group consisting of: adalimumab, certolizumab, etanercept, golimumab, infliximab, CDP571, and CDP 870.

In some embodiments, any of the antibodies or antigen-binding fragments described herein has a dissociation constant (K_{D}) of less than 1×10⁻⁵ M (e.g., less than 0.5×10⁻⁵ M, less than 1×10⁻⁶ M, less than 0.5×10⁻⁶ M, less than 1×10⁻⁷ M, less than 0.5×10⁻⁷ M, less than 1×10⁻⁸ M, less than 0.5×10⁻⁸ M, less than 1×10⁻⁹ M, less than 0.5×10⁻⁹ M, less than 1×10⁻¹⁰M, less than 0.5×10⁻¹⁰M, less than 1×10⁻¹¹ M, less than 0.5×10⁻¹¹ M, or less than 1×10⁻¹²M), e.g., as measured in phosphate buffered saline using surface plasmon resonance (SPR).

In some embodiments, any of the antibodies or antigen-binding fragments described herein has a K_{D} of about 1×10⁻¹² M to about 1×10⁻⁵ M, about 0.5×10⁻⁵ M, about 1×10⁻⁶ M, about 0.5×10⁻⁶ M, about 1×10⁻⁷ M, about 0.5×10⁻⁷ M, about 1×10⁻⁸ M, about 0.5×10⁻⁸ M, about 1×10⁻⁹ M, about 0.5×10⁻⁹ M, about 1×10⁻¹⁰M, about 0.5×10⁻¹⁰M, about 1×10⁻¹¹ M, or about 0.5×10⁻¹¹ M (inclusive); about 0.5×10⁻¹¹ M to about 1×10⁻⁵ M, about 0.5×10⁻⁵ M, about 1×10⁻⁶ M, about 0.5×10⁻⁶ M, about 1×10⁻⁷ M, about 0.5×10⁻⁷ M, about 1×10⁻⁸ M, about 0.5×10⁻⁸ M, about 1×10⁻⁹ M, about 0.5×10⁻⁹ M, about 1×10⁻¹⁰M, about 0.5×10⁻¹⁰M, or about 1×10⁻¹¹ M (inclusive); about 1×10⁻¹¹ M to about 1×10⁻⁵ M, about 0.5×10⁻⁵ M, about 1×10⁻⁶ M, about 0.5×10⁻⁶ M, about 1×10⁻⁷ M, about 0.5×10⁻⁷ M, about 1×10⁻⁸ M, about 0.5×10⁻⁸ M, about 1×10⁻⁹ M, about 0.5×10⁻⁹ M, about 1×10⁻¹⁰M, or about 0.5×10⁻¹⁰M (inclusive); about 0.5×10⁻¹⁰ M to about 1×10⁻⁵ M, about 0.5×10⁻⁵ M, about 1×10⁻⁶ M, about 0.5×10⁻⁶ M, about 1×10⁻⁷ M, about 0.5×10⁻⁷ M, about 1×10⁻⁸ M, about 0.5×10⁻⁸ M, about 1×10⁻⁹ M, about 0.5×10⁻⁹ M, or about 1×10⁻¹⁰M (inclusive); about 1×10⁻¹⁰M to about 1×10⁻⁵ M, about 0.5×10⁻⁵ M, about 1×10⁻⁶ M, about 0.5×10⁻⁶ M, about 1×10⁻⁷ M, about 0.5×10⁻⁷ M, about 1×10⁻⁸ M, about 0.5×10⁻⁸ M, about 1×10⁻⁹ M, or about 0.5×10⁻⁹ M (inclusive); about 0.5×10⁻⁹ M to about 1×10⁻⁵ M, about 0.5×10⁻⁵ M, about 1×10⁻⁶ M, about 0.5×10⁻⁶ M, about 1×10⁻⁷ M, about 0.5×10⁻⁷ M, about 1×10⁻⁸ M, about 0.5×10⁻⁸ M, or about 1×10⁻⁹ M (inclusive); about 1×10⁻⁹ M to about 1×10⁻⁵ M, about 0.5×10⁻⁵ M, about 1×10⁻⁶ M, about 0.5×10⁻⁶ M, about 1×10⁻⁷ M, about 0.5×10⁻⁷ M, about 1×10⁻⁸ M, or about 0.5×10⁻⁸ M (inclusive); about 0.5×10⁻⁸ M to about 1×10⁻⁵ M, about 0.5×10⁻⁵ M, about 1×10⁻⁶ M, about 0.5×10⁻⁶ M, about 1×10⁻⁷ M, about 0.5×10⁻⁷ M, or about 1×10⁻⁸ M (inclusive); about 1×10⁻⁸ M to about 1×10⁻⁵ M, about 0.5×10⁻⁵ M, about 1×10⁻⁶ M, about 0.5×10⁻⁶ M, about 1×10⁻⁷ M, or about 0.5×10⁻⁷ M (inclusive); about 0.5×10⁻⁷ M to about 1×10⁻⁵ M, about 0.5×10⁻⁵ M, about 1×10⁻⁶ M, about 0.5×10⁻⁶ M, or about 1×10⁻⁷ M (inclusive); about 1×10⁻⁷ M to about 1×10⁻⁵ M, about 0.5×10⁻⁵ M, about 1×10⁻⁶ M, or about 0.5×10⁻⁶ M (inclusive); about 0.5×10⁻⁶ M to about 1×10⁻⁵ M, about 0.5×10⁻⁵ M, or about 1×10⁻⁶ M (inclusive); about 1×10⁻⁶ M to about 1×10⁻⁵ M or about 0.5×10⁻⁵ M (inclusive); or about 0.5×10⁻⁵ M to about 1×10⁻⁵ M (inclusive), e.g., as measured in phosphate buffered saline using surface plasmon resonance (SPR).

In some embodiments, any of the antibodies or antigen-binding fragments described herein has a K_{off} of about 1×10⁻⁶ s⁻¹ to about 1×10⁻³ s⁻¹, about 0.5×10⁻³ s⁻¹, about 1×10⁻⁴ s⁻¹, about 0.5×10⁻⁴ s⁻¹, about 1×10⁻⁵ s⁻¹, or about 0.5×10⁻⁵ s⁻¹ (inclusive); about 0.5×10⁻⁵ s⁻¹ to about 1×10⁻³ s⁻¹, about 0.5×10⁻³ s⁻¹, about 1×10⁻⁴ s⁻¹, about 0.5×10⁻⁴ s⁻¹, or about 1×10⁻⁵ s⁻¹ (inclusive); about 1×10⁻⁵ s⁻¹ to about 1×10⁻³ s⁻¹, about 0.5×10⁻³ s⁻¹, about 1×10⁻⁴ s⁻¹, or about 0.5×10⁻⁴ s⁻¹ (inclusive); about 0.5×10⁻⁴ s⁻¹ to about 1×10⁻³ s⁻¹, about 0.5×10⁻³ s⁻¹, or about 1×10⁻⁴ s⁻¹ (inclusive); about 1×10⁻⁴ s⁻¹ to about 1×10⁻³ s⁻¹, or about 0.5×10⁻³ s⁻¹ (inclusive); or about 0.5×10⁻⁵ s⁻¹ to about 1×10⁻³ s⁻¹ (inclusive), e.g., as measured in phosphate buffered saline using surface plasmon resonance (SPR).

In some embodiments, any of the antibodies or antigen-binding fragments described herein has a Kₒₙ of about 1×10² M⁻¹s⁻¹ to about 1×10⁶ M⁻¹s⁻¹, about 0.5×10⁶ M⁻¹s⁻¹, about 1×10⁵M⁻¹s⁻¹, about 0.5×10⁵ M⁻¹s⁻¹, about 1×10⁴ M⁻¹s⁻¹, about 0.5×10⁴ M⁻¹s⁻¹, about 1×10³ M⁻¹s⁻¹, or about 0.5×10³ M⁻¹s⁻¹ (inclusive); about 0.5×10³ M⁻¹s⁻¹ to about 1×10⁶M⁻¹s⁻¹, about 0.5×10⁶ M⁻¹s⁻¹, about 1×10⁵M⁻¹s⁻¹, about 0.5×10⁵ M⁻¹s⁻¹, about 1×10⁴ M⁻¹s⁻¹, about 0.5×10⁴ M⁻¹s⁻¹, or about 1×10³ M⁻¹s⁻¹ (inclusive); about 1×10³ M⁻¹s⁻¹ to about 1×10⁶M⁻¹s⁻¹, about 0.5×10⁶ M⁻¹s⁻¹, about 1×10⁵M⁻¹s⁻¹, about 0.5×10⁵ M⁻¹s⁻¹, about 1×10⁴ M⁻¹s⁻¹, or about 0.5×10⁴ M⁻¹s⁻¹ (inclusive); about 0.5×10⁴ M⁻¹s⁻¹ to about 1×10⁶M⁻¹s⁻¹, about 0.5×10⁶ M⁻¹s⁻¹, about 1×10⁵M⁻¹s⁻¹, about 0.5×10⁵ M⁻¹s⁻¹, or about 1×10⁴ M⁻¹s⁻¹ (inclusive); about 1×10⁴ M⁻¹s⁻¹ to about 1×10⁶M⁻¹s⁻¹, about 0.5×10⁶ M⁻¹s⁻¹, about 1×10⁵M⁻¹s⁻¹, or about 0.5×10⁵ M⁻¹s⁻¹ (inclusive); about 0.5×10⁵M⁻¹s⁻¹ to about 1×10⁶M⁻¹s⁻¹, about 0.5×10⁶ M⁻¹s⁻¹, or about 1×10⁵ M⁻¹s⁻¹ (inclusive); about 1×10⁵M⁻¹s⁻¹ to about 1×10⁶M⁻¹s⁻¹, or about 0.5×10⁶ M⁻¹s⁻¹ (inclusive); or about 0.5×10⁶ M⁻¹s⁻¹ to about 1×10⁶M⁻¹s⁻¹ (inclusive), e.g., as measured in phosphate buffered saline using surface plasmon resonance (SPR).

### Fusion Proteins

In some embodiments, the anti-TNFα agent is a fusion protein (e.g., an extracellular domain of a TNFR fused to a partner peptide, e.g., an Fc region of an immunoglobulin, e.g., human IgG) (see, e.g., Deeg et al., Leukemia 16(2): 162, 2002; Peppel et al., J. Exp. Med 174(6): 1483-1489, 1991) or a soluble TNFR (e.g., TNFR1 or TNFR2) that binds specifically to TNFα. In some embodiments, the anti-TNFα agent includes or is a soluble TNFα receptor (e.g., Bjornberg et al., Lymphokine Cytokine Res. 13(3):203-211, 1994; Kozak et al., Am. J. Physiol. Reg. Integrative Comparative Physiol. 269(1):R23-R29, 1995; Tsao et al., Eur Respir J. 14(3):490-495, 1999; Watt et al., J Leukoc Biol. 66(6): 1005-1013, 1999; Mohler et al., J. Immunol. 151(3):1548-1561, 1993; Nophar et al., EMBO J. 9(10):3269, 1990; Piguet et al., Eur. Respiratory J. 7(3):515-518, 1994; and Gray et al., Proc. Natl. Acad Sci. U.S.A. 87(19):7380-7384, 1990). In some embodiments, the anti-TNFα agent includes or is etanercept (Enbrel™) (see, e.g., WO 91/03553 and WO 09/406,476, incorporated by reference herein). In some embodiments, the anti-TNFα agent inhibitor includes or is r-TBP-I (e.g., Gradstein et al., J. Acquir. Immune Defic. Syndr. 26(2): 111-117, 2001).

### Inhibitory Nucleic Acids

Inhibitory nucleic acids that can decrease the expression of AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP mRNA expression in a mammalian cell include antisense nucleic acid molecules, i.e., nucleic acid molecules whose nucleotide sequence is complementary to all or part of a AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP mRNA (e.g., complementary to all or a part of any one of SEQ ID NOs: 1-37).

The nucleotides characterized by the Sequences ID NO: 1-37 are listed below and are being submitted in a separate and machine readable file.

List of Nucleotides with Sequence SEQ ID NO: 1-37

Human TNFα CDS (SEQ ID NO: 1), Human TNFR1 CDS (SEQ ID NO: 2), Human TNFR2 CDS (SEQ ID NO: 3), Human TRADD CDS (SEQ ID NO: 4), Human TRAF2 CDS (SEQ ID NO: 5), Human AP-1 CDS (SEQ ID NO: 6), Human ASK1 CDS (SEQ ID NO: 7), Human CD14 CDS (SEQ ID NO: 8), Human ERK1 CDS (SEQ ID NO: 9), Human ERK2 CDS (SEQ ID NO: 10), Human IKK CDS (SEQ ID NO: 11), Human IκB CDS (SEQ ID NO: 12), Human IRAK CDS (SEQ ID NO: 13), Human JNK CDS (SEQ ID NO: 14), Human LBP CDS (SEQ ID NO: 15), Human MEK1 CDS (SEQ ID NO: 16), Human MEK2 CDS (SEQ ID NO: 17), Human MEK3 CDS (SEQ ID NO: 18), Human MEK6 CDS (SEQ ID NO: 19), Human MEKK1 CDS (SEQ ID NO: 20), Human MEKK 3 CDS (SEQ ID NO: 21), Human MEKK4 CDS (SEQ ID NO: 22), Human MEKK 6 CDS (SEQ ID NO: 23), Human MEKK7 CDS (SEQ ID NO: 24), Human MK2 CDS (SEQ ID NO: 25), Human MyD88 CDS (SEQ ID NO: 26), Human NF-κB CDS (SEQ ID NO: 27), Human NIK CDS (SEQ ID NO: 28), Human p38 CDS (SEQ ID NO: 29), Human PKR CDS (SEQ ID NO: 30), Human Rac CDS (SEQ ID NO: 31), Human Raf CDS (SEQ ID NO: 32), Human K-Ras CDS (SEQ ID NO: 33), Human N-Ras CDS (SEQ ID NO: 34), Human RIP CDS (SEQ ID NO: 35), Human TRAF6 CDS (SEQ ID NO: 36), and Human TTP CDS (SEQ ID NO: 37).

An antisense nucleic acid molecule can be complementary to all or part of a non-coding region of the coding strand of a nucleotide sequence encoding an AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTPMEKK1 protein. Non-coding regions (5′ and 3′ untranslated regions) are the 5′ and 3′ sequences that flank the coding region in a gene and are not translated into amino acids.

Based upon the sequences disclosed herein, one of skill in the art can easily choose and synthesize any of a number of appropriate antisense nucleic acids to target a nucleic acid encoding an AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP protein described herein. Antisense nucleic acids targeting a nucleic acid encoding an AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTPMEKK1 protein can be designed using the software available at the Integrated DNA Technologies website.

An antisense nucleic acid can be, for example, about 5, 10, 15, 18, 20, 22, 24, 25, 26, 28, 30, 32, 35, 36, 38, 40, 42, 44, 45, 46, 48, or 50 nucleotides or more in length. An antisense oligonucleotide can be constructed using enzymatic ligation reactions and chemical synthesis using procedures known in the art. For example, an antisense nucleic acid can be chemically synthesized using variously modified nucleotides or naturally occurring nucleotides designed to increase the physical stability of the duplex formed between the antisense and sense nucleic acids, e.g., phosphorothioate derivatives and acridine substituted nucleotides or to increase the biological stability of the molecules.

Examples of modified nucleotides which can be used to generate an antisense nucleic acid include 1-methylguanine, 1-methylinosine, 2,2-dimethylguanine, 2-methyladenine, 2-methylguanine, 3-methylcytosine, 2-methylthio-N6-isopentenyladenine, uracil-5-oxyacetic acid (v), wybutoxosine, pseudouracil, queosine, 2-thiocytosine, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest).

The antisense nucleic acid molecules described herein can be prepared in vitro and administered to a subject, e.g., a human subject. Alternatively, they can be generated in situ such that they hybridize with or bind to cellular mRNA and/or genomic DNA encoding an AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP protein to thereby inhibit expression, e.g., by inhibiting transcription and/or translation. The hybridization can be by conventional nucleotide complementarities to form a stable duplex, or, for example, in the case of an antisense nucleic acid molecule that binds to DNA duplexes, through specific interactions in the major groove of the double helix. The antisense nucleic acid molecules can be delivered to a mammalian cell using a vector (e.g., an adenovirus vector, a lentivirus, or a retrovirus).

An antisense nucleic acid can be an α-anomeric nucleic acid molecule. An α-anomeric nucleic acid molecule forms specific double-stranded hybrids with complementary RNA in which, contrary to the usual, β-units, the strands run parallel to each other (Gaultier et al., Nucleic Acids Res. 15:6625-6641, 1987). The antisense nucleic acid can also comprise a chimeric RNA-DNA analog (Inoue et al., FEBS Lett. 215:327-330, 1987) or a 2′-O-methylribonucleotide (Inoue et al., Nucleic Acids Res. 15:6131-6148, 1987).

Another example of an inhibitory nucleic acid is a ribozyme that has specificity for a nucleic acid encoding an AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP mRNA, e.g., specificity for any one of SEQ ID NOs: 1-37). Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes (described in Haselhoff and Gerlach, Nature 334:585-591, 1988)) can be used to catalytically cleave mRNA transcripts to thereby inhibit translation of the protein encoded by the mRNA. An AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP mRNA can be used to select a catalytic RNA having a specific ribonuclease activity from a pool of RNA molecules. See, e.g., Bartel et al., Science 261:1411-1418, 1993.

Alternatively, a ribozyme having specificity for an AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP mRNA can be designed based upon the nucleotide sequence of any of the AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP mRNA sequences disclosed herein. For example, a derivative of a Tetrahymena L-19 IVS RNA can be constructed in which the nucleotide sequence of the active site is complementary to the nucleotide sequence to be cleaved in an AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP mRNA (see, e.g, U.S. Pat. Nos. 4,987,071 and 5,116,742).

An inhibitory nucleic acid can also be a nucleic acid molecule that forms triple helical structures. For example, expression of an AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP polypeptide can be inhibited by targeting nucleotide sequences complementary to the regulatory region of the gene encoding the AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP polypeptide (e.g., the promoter and/or enhancer, e.g., a sequence that is at least 1 kb, 2 kb, 3 kb, 4 kb, or 5 kb upstream of the transcription initiation start state) to form triple helical structures that prevent transcription of the gene in target cells. See generally Maher, Bioassays 14(12):807-15, 1992; Helene, Anticancer Drug Des. 6(6):569-84, 1991; and Helene, Ann. N.Y. Acad Set. 660:27-36, 1992.

In various embodiments, inhibitory nucleic acids can be modified at the sugar moiety, the base moiety, or phosphate backbone to improve, e.g., the solubility, stability, or hybridization, of the molecule. For example, the deoxyribose phosphate backbone of the nucleic acids can be modified to generate peptide nucleic acids (see, e.g., Hyrup et al., Bioorganic Medicinal Chem. 4(1):5-23, 1996). Peptide nucleic acids (PNAs) are nucleic acid mimics, e.g., DNA mimics, in which the deoxyribose phosphate backbone is replaced by a pseudopeptide backbone and only the four natural nucleobases are retained. The neutral backbone of PNAs allows for specific hybridization to RNA and DNA under conditions of low ionic strength. PNA oligomers can be synthesized using standard solid phase peptide synthesis protocols (see, e.g., Perry-O'Keefe et al., Proc. Natl. Acad. Set. U.S.A. 93:14670-675, 1996). PNAs can be used as antisense or antigene agents for sequence-specific modulation of gene expression by, e.g., inducing transcription or translation arrest or inhibiting replication.

### Small Molecules

In some embodiments, the anti-TNFα agent is a small molecule. In some embodiments, the small molecule is a tumor necrosis factor-converting enzyme (TACE) inhibitor (e.g., Moss et al., Nature Clinical Practice Rheumatology 4: 300-309, 2008). In some embodiments, the anti-TNFα agent is C87 (Ma et al., J. Biol. Chem. 289(18): 12457-66, 2014). In some embodiments, the small molecule is LMP-420 (e.g., Haraguchi et al., AIDS Res. Ther. 3:8, 2006). In some embodiments, the TACE inhibitor is TMI-005 and BMS-561392. Additional examples of small molecule inhibitors are described in, e.g., He et al., Science 310(5750): 1022-1025, 2005.

In some examples, the anti-TNFα agent is a small molecule that inhibits the activity of one of AP-1, ASK1, IKK, JNK, MAPK, MEKK 1/4, MEKK4/7, MEKK 3/6, NIK, TRADD, RIP, NF-κB, and TRADD in a cell (e.g., in a cell obtained from a subject, a mammalian cell).

In some examples, the anti-TNFα agent is a small molecule that inhibits the activity of one of CD14, MyD88 (see, e.g., Olson et al., Scientific Reports 5:14246, 2015), ras (e.g., Baker et al., Nature 497:577-578, 2013), raf (e.g., vemurafenib (PLX4032, RG7204), sorafenib tosylate, PLX-4720, dabrafenib (GSK2118436), GDC-0879, RAF265 (CHIR-265), AZ 628, NVP-BHG712, SB590885, ZM 336372, sorafenib, GW5074, TAK-632, CEP-32496, encorafenib (LGX818), CCT196969, LY3009120, R05126766 (CH5126766), PLX7904, and MLN2480).

In some examples, the anti-TNFα agent TNFα inhibitor is a small molecule that inhibits the activity of one of MK2 (PF 3644022 and PHA 767491), JNK (e.g., AEG 3482, BI 78D3, CEP 1347, c-JUN peptide, IQ IS, JIP-1 (153-163), SP600125, SU 3327, and TCS JNK6o), c-jun (e.g., AEG 3482, BI 78D3, CEP 1347, c-JUN peptide, IQ 1S, JIP-1 (153-163), SP600125, SU 3327, and TCS JNK6o), MEK3/6 (e.g., Akinleye et al., J. Hematol. Oncol. 6:27, 2013), p38 (e.g., AL 8697, AMG 548, BIRB 796, CMPD-1, DBM 1285 dihydrochloride, EO 1428, JX 401, ML 3403, Org 48762-0, PH 797804, RWJ 67657, SB 202190, SB 203580, SB 239063, SB 706504, SCIO 469, SKF 86002, SX 011, TA 01, TA 02, TAK 715, VX 702, and VX 745), PKR (e.g., 2-aminopurine or CAS 608512-97-6), TTP (e.g., CAS 329907-28-0), MEK1/2 (e.g., Facciorusso et al., Expert Review Gastroentrol. Hepatol. 9:993-1003, 2015), ERK1/2 (e.g., Mandal et al., Oncogene 35:2547-2561, 2016), NIK (e.g., Mortier et al., Bioorg. Med Chem. Lett. 20:4515-4520, 2010), IKK (e.g., Reilly et al., Nature Med 19:313-321, 2013), IκB (e.g., Suzuki et al., Expert. Opin. Invest. Drugs 20:395-405, 2011), NF-κB (e.g., Gupta et al., Biochim. Biophys. Acta 1799(10-12):775-787, 2010), rac (e.g., U.S. Pat. No. 9,278,956), MEK4/7, IRAK (Chaudhary et al., J. Med. Chem. 58(1):96-110, 2015), LBP (see, e.g., U.S. Pat. No. 5,705,398), and TRAF6 (e.g., 3-[(2,5-Dimethylphenyl)amino]-1-phenyl-2-propen-1-one).

In some embodiments of any of the methods described herein, the inhibitory nucleic acid can be about 10 nucleotides to about 50 nucleotides (e.g., about 10 nucleotides to about 45 nucleotides, about 10 nucleotides to about 40 nucleotides, about 10 nucleotides to about 35 nucleotides, about 10 nucleotides to about 30 nucleotides, about 10 nucleotides to about 28 nucleotides, about 10 nucleotides to about 26 nucleotides, about 10 nucleotides to about 25 nucleotides, about 10 nucleotides to about 24 nucleotides, about 10 nucleotides to about 22 nucleotides, about 10 nucleotides to about 20 nucleotides, about 10 nucleotides to about 18 nucleotides, about 10 nucleotides to about 16 nucleotides, about 10 nucleotides to about 14 nucleotides, about 10 nucleotides to about 12 nucleotides, about 12 nucleotides to about 50 nucleotides, about 12 nucleotides to about 45 nucleotides, about 12 nucleotides to about 40 nucleotides, about 12 nucleotides to about 35 nucleotides, about 12 nucleotides to about 30 nucleotides, about 12 nucleotides to about 28 nucleotides, about 12 nucleotides to about 26 nucleotides, about 12 nucleotides to about 25 nucleotides, about 12 nucleotides to about 24 nucleotides, about 12 nucleotides to about 22 nucleotides, about 12 nucleotides to about 20 nucleotides, about 12 nucleotides to about 18 nucleotides, about 12 nucleotides to about 16 nucleotides, about 12 nucleotides to about 14 nucleotides, about 15 nucleotides to about 50 nucleotides, about 15nucleotides to about 45 nucleotides, about 15nucleotides to about 40 nucleotides, about 15nucleotides to about 35 nucleotides, about 15 nucleotides to about 30 nucleotides, about 15nucleotides to about 28 nucleotides, about 15nucleotides to about 26 nucleotides, about 15nucleotides to about 25 nucleotides, about 15nucleotides to about 24 nucleotides, about 15nucleotides to about 22 nucleotides, about 15nucleotides to about 20 nucleotides, about 15nucleotides to about 18 nucleotides, about 15nucleotides to about 16 nucleotides, about 16 nucleotides to about 50 nucleotides, about 16 nucleotides to about 45 nucleotides, about 16 nucleotides to about 40 nucleotides, about 16 nucleotides to about 35 nucleotides, about 16 nucleotides to about 30 nucleotides, about 16 nucleotides to about 28 nucleotides, about 16 nucleotides to about 26 nucleotides, about 16 nucleotides to about 25 nucleotides, about 16 nucleotides to about 24 nucleotides, about 16 nucleotides to about 22 nucleotides, about 16 nucleotides to about 20 nucleotides, about 16 nucleotides to about 18 nucleotides, about 18 nucleotides to about 20 nucleotides, about 20 nucleotides to about 50 nucleotides, about 20 nucleotides to about 45 nucleotides, about 20 nucleotides to about 40 nucleotides, about 20 nucleotides to about 35 nucleotides, about 20 nucleotides to about 30 nucleotides, about 20 nucleotides to about 28 nucleotides, about 20 nucleotides to about 26 nucleotides, about 20 nucleotides to about 25 nucleotides, about 20 nucleotides to about 24 nucleotides, about 20 nucleotides to about 22 nucleotides, about 24 nucleotides to about 50 nucleotides, about 24 nucleotides to about 45 nucleotides, about 24 nucleotides to about 40 nucleotides, about 24 nucleotides to about 35 nucleotides, about 24 nucleotides to about 30 nucleotides, about 24 nucleotides to about 28 nucleotides, about 24 nucleotides to about 26 nucleotides, about 24 nucleotides to about 25 nucleotides, about 26 nucleotides to about 50 nucleotides, about 26 nucleotides to about 45 nucleotides, about 26 nucleotides to about 40 nucleotides, about 26 nucleotides to about 35 nucleotides, about 26 nucleotides to about 30 nucleotides, about 26 nucleotides to about 28 nucleotides, about 28 nucleotides to about 50 nucleotides, about 28 nucleotides to about 45 nucleotides, about 28 nucleotides to about 40 nucleotides, about 28 nucleotides to about 35 nucleotides, about 28 nucleotides to about 30 nucleotides, about 30 nucleotides to about 50 nucleotides, about 30 nucleotides to about 45 nucleotides, about 30 nucleotides to about 40 nucleotides, about 30 nucleotides to about 38 nucleotides, about 30 nucleotides to about 36 nucleotides, about 30 nucleotides to about 34 nucleotides, about 30 nucleotides to about 32 nucleotides, about 32 nucleotides to about 50 nucleotides, about 32 nucleotides to about 45 nucleotides, about 32 nucleotides to about 40 nucleotides, about 32 nucleotides to about 35 nucleotides, about 35 nucleotides to about 50 nucleotides, about 35 nucleotides to about 45 nucleotides, about 35 nucleotides to about 40 nucleotides, about 40 nucleotides to about 50 nucleotides, about 40 nucleotides to about 45 nucleotides, about 42 nucleotides to about 50 nucleotides, about 42 nucleotides to about 45 nucleotides, or about 45 nucleotides to about 50 nucleotides) in length. One skilled in the art will appreciate that inhibitory nucleic acids may comprises at least one modified nucleic acid at either the 5′ or 3′ end of DNA or RNA.

In some embodiments, the inhibitory nucleic acid can be formulated in a liposome, a micelle (e.g., a mixed micelle), a nanoemulsion, or a microemulsion, a solid nanoparticle, or a nanoparticle (e.g., a nanoparticle including one or more synthetic polymers). Additional exemplary structural features of inhibitory nucleic acids and formulations of inhibitory nucleic acids are described in US 2016/0090598.

In some embodiments, the inhibitory nucleic acid (e.g., any of the inhibitory nucleic acid described herein) can include a sterile saline solution (e.g., phosphate-buffered saline (PBS)). In some embodiments, the inhibitory nucleic acid (e.g., any of the inhibitory nucleic acid described herein) can include a tissue-specific delivery molecule (e.g., a tissue-specific antibody).

Compound Preparation and Biological Assays

As can be appreciated by the skilled artisan, methods of synthesizing the compounds of the formulae herein will be evident to those of ordinary skill in the art. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and RGM. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

### Preparative Examples

### Abbreviation of Chemicals

ACN=acetonitrile
AcOH=acetic acid
BTC=trichloromethyl chloroformate
DBU=1,8-diazabicycloundec-7-ene
DCM=dichloromethane
Dess-Martin=(1,1,1-triacetoxy)-1,1-dihydro-1,2-benziodoxol-3(1H)-one
DMEDA=N,N′-dinethylethylenediamine

### DMF=N,N-dimethylformamide

DMSO=dimethyl sulfoxide
Et=ethyl
EtOH=ethanol
LC-MS=liquid chromatography-mass spectrometry
LDA=lithum diisopropylamide
Me=methyl
MeOH=methanol
n-Bu=n-butyl

### NBS=N-bromosuccinimide

### NCS=N-chlorosuccinimide

### NIS=N-iodosuccinimide

NMR=nuclear magnetic resonance
PE=petroleum ether
Pd(dppf)Cl₂=dichloro[1,1′-bis(diphenylphosphino)ferrocene]palladium
Pd(PPh₃)₄=tetrakis(triphenylphosphine)Palladium(0)
Ph=phenyl
HPLC=high performance liquid chromatography
PTSA=p-toluenesulfonic acid
Py=pyridine
RT=room temperature
TBAF=tetrabutylammonium fluoride
TBDPSCl=tert-butyldiphenylsilyl chloride
t-Bu=tert-butyl
TEA=triethylamine
TFA=trifluoroacetic acid
THE=tetrahydrofuran
Ti(i-PrO)₄=tetraisopropyl titanate
TLC=thin layer chromatography

### Materials and Methods

The progress of reactions was often monitored by TLC or LMC-S. The identity of the products was often confirmed by LC-MS. The LC-MS was recorded using one of the following methods.

Method A: Shim-pack XR-ODS, C I8, 3×50 mm, 2.5 um column, 1.0 uL injection, 1.5 mL/min flow rate, 90-900 amu scan range, 190-400 nm UV range, 5-100% (11 min), 100% (0.6 min) gradient with ACN (0.05% TFA) and water (0.05% TFA), 2 minute total run time.

Method 13: Kinetex EVO, C18, 3×50 mm, 2.2 um column, 1.0 uL injection, 15 mL/min flow rate, 90-900 amu scan range, 190-400 nm UV range, 10-95% (1.1 min), 95% (0.6 min) gradient with ACN and water (0.5% NH₄HCO₃), 2 minute total run time.

Method C: Shim-pack X-ODS, C18, 3×50 mm, 2.5 um column, 1.0 uL injection, 1.5 mL/min flow rate, 90-900 amu scan range, 190-400 nm UV range, 5-100% (2.1 min), 100% (0.6 min) gradient with ACN (0.05% TFA) and water (0.05% TFA), 3 minute total run time.

Method D: Kinetex EVO, C18, 3×50 mm, 2.2 um column, 1.0 uL injection, 1.5 mL/min flow rate, 90-900 amu scan range, 190-400 nm UV range, 10-95% (2.1 min), 95% (0.6 min) gradient with ACN and water (0.5% NH₄HCO₃), 3 minute total run time.

Method E: CORTECS C18+, 50*2.1 mm, 0.5 uL injection, 0.8 mL/min flowrate, 90-900 amu scan range, 254 nm UV detection. Mobile phase A: Water (0.1% FA) and Mobile Phase B: MeCN. 10% MPB to 95.0% in 1.1 min, hold at 95% MPB for 0.5 min, 95% MPB to 10% in 0.03 min, then equilibration to 10% MPB for 0.2 min, 1.8 minute total run time.

Method F: YMC Triart-C18, 50*3.0 mm, 1.0 uL injection, 1.0 mL/min flowrate, 90-900 amu scan range, 254 nm UV detection. Mobile phase A: Water (5 mmoL/L NH4HCO3) and Mobile Phase B: MeCN. 10% MPB to 95.0% in 1.1 min, hold at 95% MPB for 0.5 min, 95% MPB to 10% in 0.1 min, then equilibration to 10% MPB for 0.1 min, 1.8 minute total run time

Method G: Agilent Poroshell HPH, 50*3.0 mm, 0.8 uL injection, 1.0 mL/min flowrate, 90-900 amu scan range, 254 nm UV detection. Mobile phase A: Water (5 mmoL/L NH4HCO3) and Mobile Phase B: MeCN. 10% MPB to 95.0% in 1.1 min, hold at 95% MPB for 0.5 min, 95% MPB to 10% in 0.1 min, then equilibration to 10% MPB for 0.1 min, 1.8 minute total run time

Method I: Kinetex EVO, C18, 3×50 mm, 2.2 um column, 0.3 uL injection, 1.5 mL/min flow rate, 90-900 amu scan range, 190-400 nm UV range, Mobile phase A: Water (5 mmoL/L NH4HCO3) and Mobile Phase B: MeCN. 10% MPB to 95.0% in 1.1 min, hold at 95% MPB for 0.5 min, 95% MPB to 10% in 0.1 min, then equilibration to 10% MPB for 0.1 min, 1.8 minute total run time

Method J: Kinetex EVO, 50*3.0 mm, 0.5 uL injection, 1.2 mL/min flowrate, 90-900 amu scan range, 254 nm UV detection. Mobile phase A: Water (0.03% NH3H2O) and Mobile Phase B: MeCN. 10% MPB to 95.0% in 2.0 min, hold at 95% MPB for 0.6 min, 95% MPB to 10% in 0.15 min, then equilibration to 10% MPB for 0.25 min.

Method K: YMC Triart-C18, 50*3.0 mm, 1.0 uL injection, 1.0 mL/min flowrate, 90-900 amu scan range, 254 nm UV detection. Mobile phase A: Water (0.5% NH4HCO3) and Mobile Phase B: MeCN. 10% MPB to 95.0% in 2.1 min, hold at 95% MPB for 0.6 min, 95% MPB to 10% in 0.1 min, then equilibration to 10% MPB for 0.2 min.

Method L: Kinetex@ 2.6 um EVO C18100 A, 50*3.0 mm, 0.3 uL injection, 1.5 mL/min flowrate, 90-900 amu scan range, 254 nm UV detection. Mobile phase A: Water (5 mmoL/L NH4HCO3) and Mobile Phase B: MeCN. 10% MPB to 95.0% in 2.0 min, hold at 95% MPB for 0.79 min, 95% MPB to 10% in 0.06 min, then equilibration to 10% MPB for 0.15 min.

Method M: Kinetex@ 2.6 um EVO C18100 A, 50*3.0 mm, 0.3 uL injection, 1.5 mL/min flowrate, 90-900 amu scan range, 254 nm UV detection. Mobile phase A: Water (5 mmoL/L NH4HCO3) and Mobile Phase B: MeCN. 10% MPB to 95.0% in 1.6 min, hold at 95% MPB for 0.8 min, 95% MPB to 10% in 0.6 min, then equilibration to 10% MPB for 0.14 min.

Method N: Agilent Poroshell HPH-C18, 50*3 mm, 2.7 um column, 4.0 uL injection, 1.0 mL/min flow rate, 90-900 amu scan range, 190-400 nm UV range, Mobile phase A: Water (5 mmoL/L NH4HCO3) and Mobile Phase B: MeCN. 10% MPB to 95% in 2.1 min, hold at 95% MPB for 0.6 min, 95% MPB to 10% in 0.1 min, then equilibration to 10% MPB for 0.2 min.

The final targets were purified by Prep-HPLC. The Prep-HPLC was carried out using the following method

Method O: Pre-HPLC: Column, XBridge Shield RP18 OBD (19×250 mm, 10 um); mobile phase, Water (10 mmol/L NH4HCO3) and ACN, UV detection 254/210 nm.

NMR was recorded on BRUKER NMR 300.03 Mz, DUL-C-H, ULTRASHIELD™ 300, AVANCE 11300 B-ACS™ 120 or BRUKER NMR 400.13 Mz, BBFO, ULTRASHIELD™ 400, AVANCE III 400, B-ACS™ 120.

### Preparative Examples

Scheme for the preparation of Sulfonamides Intermediates: Schemes below illustrate the preparation of sulfonamide intermediates.

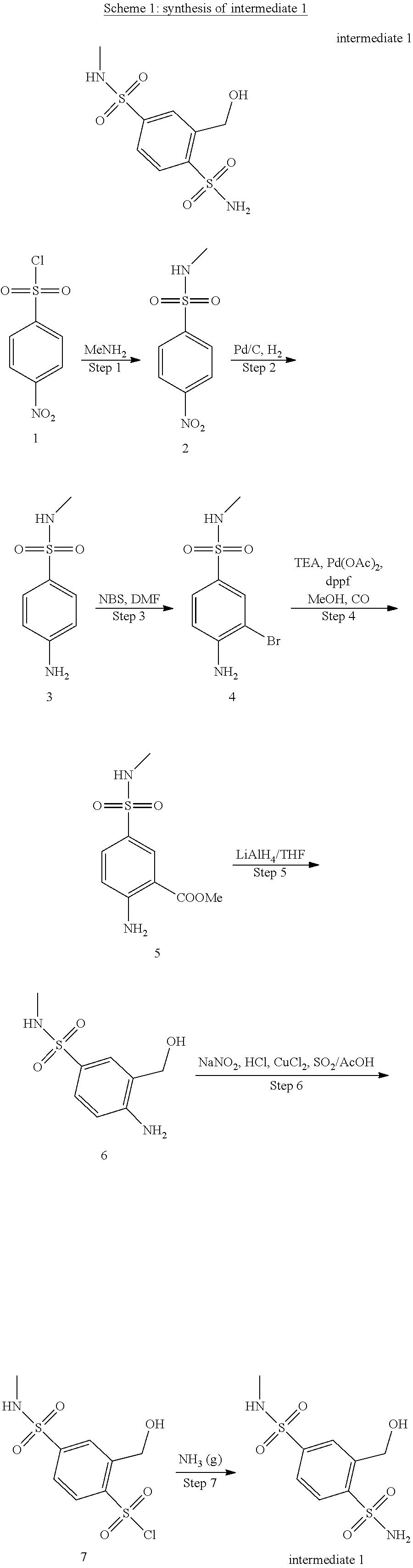

### 1. Synthesis of N-methyl-4-nitrobenzene-1-sulfonamide

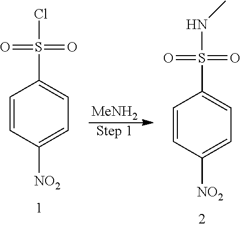

Into a 250 mL round-bottom flask were added methanamine (91 mL, 54.2 mmol, 2 equiv) at room temperature. To the stirred liquid was added 4-nitrobenzene-1-sulfonyl chloride (7.0 g, 31.7 mmol, 1 equiv) in portions at 0° C. Then the resulting mixture was stirred for 1 h at room temperature. The residue was purified by silica gel column chromatography, eluted with PE/EtOAc (2:1) to afford N-methyl-4-nitrobenzene-1-sulfonamide (5.8 g, 84.7%) as a light yellow solid.

### 2. Synthesis of 4-amino-N-methylbenzene-1-sulfonamide

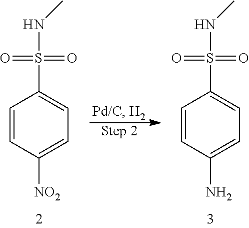

Into a 250 mL round-bottom flask were added N-methyl-4-nitrobenzene-1-sulfonamide (5.8 g, 26.8 mmol, 1 equiv) and isopropanol (50 mL) at room temperature. To a stirred solution of N-methyl-4-nitrobenzene-1-sulfonamide (5.8 g, 26.8 mmol, 1 equiv) in isopropanol (50 mL) was added Pd/C (580 mg, 5.5 mmol, 0.20 equiv) at room temperature under nitrogen. The resulting mixture was stirred overnight at room temperature under hydrogen atmosphere, and was then filtered. The filtrate was concentrated under reduced pressure to afforded 4-amino-N-methylbenzene-1-sulfonamide (4.9 g, 84.5%) as yellow solid.

LCMS of 4-amino-N-methylbenzene-1-sulfonamide (Method B): 187 [M+H]⁺, retention time 0.625 min.

### 3. Synthesis of 4-amino-3-bromo-N-methylbenzene-1-sulfonamide

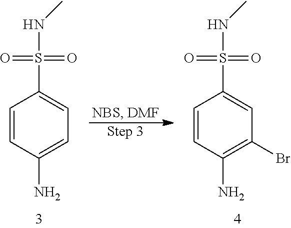

Into a 100 mL round-bottom flask were added 4-amino-3-(hydroxymethyl)-N-methylbenzene-1-sulfonamide (5.8 g, 26.8 mmol, 1 equiv) and DMF (25 mL) at room temperature. To a stirred solution of 4-amino-N-methylbenzene-1-sulfonamide (5.8 g, 26.8 mmol, 1 equiv) in DMF (25 mL) was added NBS (4.3 g, 24.1 mmol, 0.9 equiv) in portions at room temperature. The residue was purified by silica gel column chromatography, eluted with PE/EtOAc (2:1) to afford 4-amino-3-bromo-N-methylbenzene-1-sulfonamide (6 g, 84.4%) as a dark yellow solid.

LCMS of 4-amino-3-bromo-N-methylbenzene-1-sulfonamide (Method B): 265, 267 [M+H]⁺, retention time 0.863 min.

### 4. Synthesis of methyl 2-amino-5-(methylsulfamoyl)benzoate

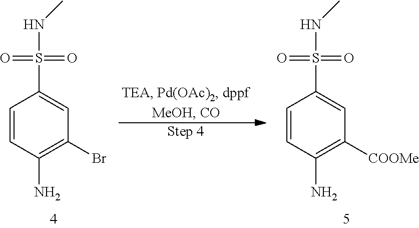 [SMILES: CNS(=O)(=O)c1ccc(N)c(Br)c1 ; "Step 4" ; "TEA, Pd(OAc)2, dppfMeOH, CO" ; CNS(=O)(=O)c1ccc(N)c(C(=O)OC)c1]

Into a 250 mL pressure tank reactor were added 4-amino-3-bromo-N-methylbenzene-1-sulfonamide (6.0 g, 22.6 mmol, 1 equiv) and TEA (2.2 g, 22.6 mmol, 1 equiv) at room temperature. To a stirred solution of 4-amino-3-bromo-N-methylbenzene-1-sulfonamide (6.0 g, 22.6 mmol, 1 equiv) and TEA (2.2 mg, 22.6 mmol, 1 equiv) in MeOH(150 mL) were added Pd(OAc)₂ (1.0 g, 4.5 mmol, 0.2 equiv) and dppf (3.8 g, 6.8 mmol, 0.3 equiv) in one portion under N2 atmosphere. Then the resulting mixture was stirred at 110° C. under CO atmosphere (10 atm) overnight. The resulting mixture was concentrated under reduced pressure. The residue was purified by silica gel column chromatography, eluted with PE/EtOAc (2:1) to afford methyl 2-amino-5-(methylsulfamoyl)benzoate(4.7 g, 74.4%) as a light yellow solid.

LCMS of methyl 2-amino-5-(methylsulfamoyl)benzoate (Method B): 245 [M+H]⁺, retention time 0.854 min.

### 5. Synthesis of 4-amino-3-(hydroxymethyl)-N-methylbenzene-1-sulfonamide

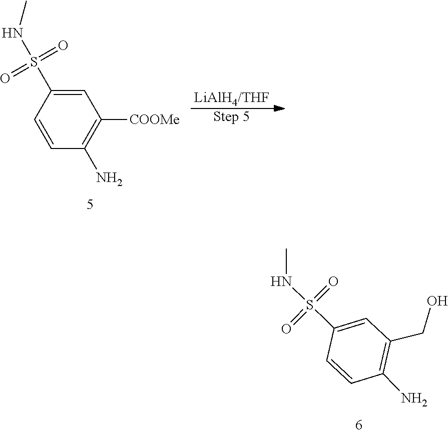 [SMILES: CNS(=O)(=O)c1ccc(N)c(C(=O)OC)c1 ; "Step 5" ; "LiAlH4/THF" ; CNS(=O)(=O)c1ccc(N)c(CO)c1]

Into a 500 mL round-bottom flask were added methyl 2-amino-5-(methylsulfamoyl)benzoate (4.5 g, 18.4 mmol, 1 equiv) and THF (100 mL) at room temperature. To a stirred solution of methyl 2-amino-5-(methylsulfamoyl)benzoate (4.5 g, 18.4 mmol, 1 equiv) in THF(100 mL) were added LiAlH₄ (1398.4 mg, 36.84 mmol, 2 equiv) in portions at 0° C. under nitrogen atmosphere. Then the resulting mixture was stirred for 4 h. The resulting mixture was concentrated under reduced pressure. The crude product was purified by reverse phase flash with the following conditions (column, C18 silica gel; mobile phase, acetonitrile in water, 0% to 15% gradient in 7 min) to afford 4-amino-3-(hydroxymethyl)-N-methylbenzene-1-sulfonamide 2.2 g (55.3%) as a light yellow solid.

LCMS of 4-amino-3-(hydroxymethyl)-N-methylbenzene-1-sulfonamide (Method B): 217 [M+H]⁺, retention time 0.472 min.

### 6. Synthesis of 2-(hydroxymethyl)-4-(N-methylsulfamoyl)benzene-1-sulfonyl chloride

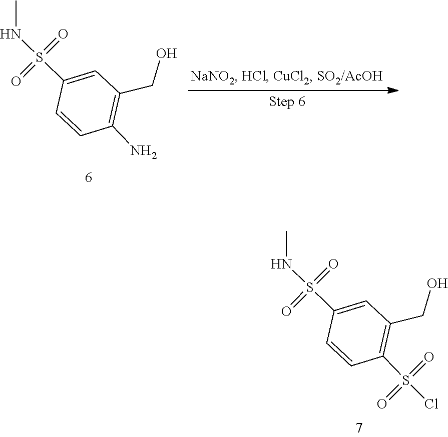 [SMILES: CNS(=O)(=O)c1ccc(N)c(CO)c1 ; CNS(=O)(=O)c1ccc(S(=O)(=O)Cl)c(CO)c1]

Into a 50 mL 3-necked round-bottom flask were added 4-amino-3-(hydroxymethyl)-N-methylbenzene-1-sulfonamide (1 g, 4.62 mmol, 1 equiv) at room temperature. To a stirred solution of 4-amino-3-(hydroxymethyl)-N-methylbenzene-1-sulfonamide (1 g, 4.62 mmol, 1 equiv) in HCl (6M) (10 mL) was added NaNO₂ (382.8 mg, 5.55 mmol, 1.20 equiv) dropwise at −10 degrees C. for 20 min. Then the resulting mixture was added to the solution of CuCl₂ in SO₂/AcOH(15 mL) (that had been stirred together for 15 min) in one portion at −10 degrees C. for 30 min. The resulting mixture was diluted with water (50 mL) and was extracted with CH₂ Cl₂ (3×25 mL). The combined organic layers were washed with water (3×50 mL), dried over anhydrous Na₂SO₄, and filtered. The filtrate was concentrated under reduced pressure. The crude product was used in the next step directly without further purification.

### 7. Synthesis of 3-(hydroxymethyl)-N1-methylbenzene-1,4-disulfonamide

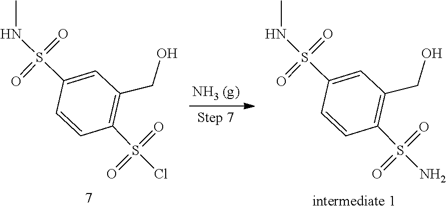 [SMILES: CNS(=O)(=O)c1ccc(S(=O)(=O)Cl)c(CO)c1 ; CNS(=O)(=O)c1ccc(S(N)(=O)=O)c(CO)c1]

Into a 100 mL round-bottom flask were added NH3 in THE (40 mL, 0.5M) at 0 degrees C. To a stirred solution of NH3 in THF(40 mL) was added 2-(hydroxymethyl)-4-(methylsulfamoyl)benzene-1-sulfonyl chloride (1 g, 3.34 mmol, 1 equiv) in THF (6 mL) dropwise at 0 degrees C. The resulting mixture was stirred overnight at room temperature. The residue was purified by Prep-TLC (EtOAc) to afford 3-(hydroxymethyl)-N1-methylbenzene-1,4-disulfonamide (400 mg, 42.7%) as a yellow solid.

LCMS of 4-amino-3-(hydroxymethyl)-N-methylbenzene-1-sulfonamide (Method B): 279 [M−H]−, retention time 0.542 min.

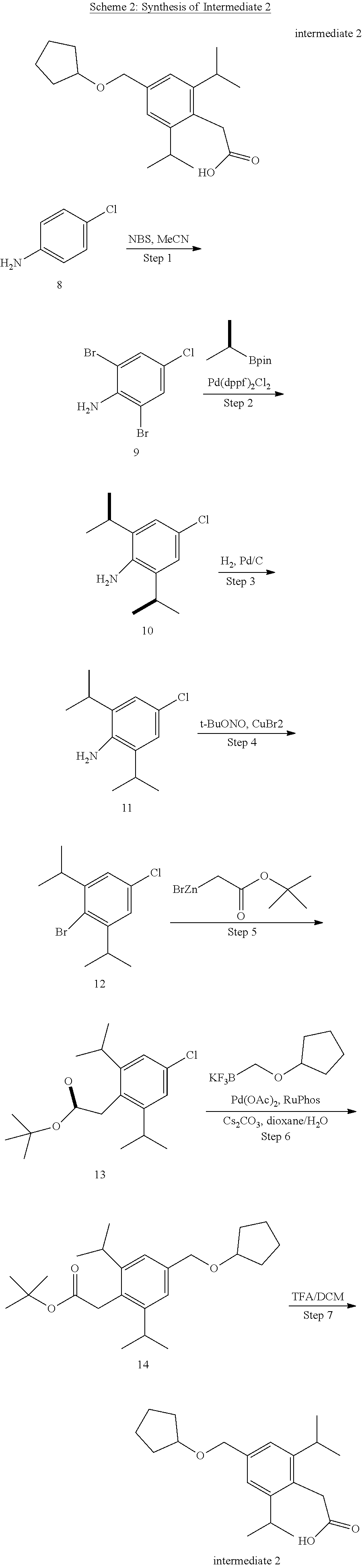

### 1. Synthesis of 2,6-dibromo-4-chlorobenzenamine

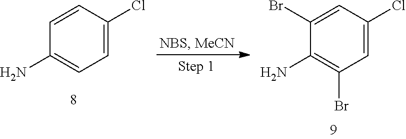 [SMILES: Nc1ccc(Cl)cc1 ; Nc1c(Br)cc(Cl)cc1Br]

Into a 250-mL round-bottom flask, was placed a solution of 4-chlorobenzenamine (12.7 g, 100 mmol, 1 equiv) in MeCN (200 mL). NBS (44.5 g, 250 mmol, 2.5 equiv) was added to the solution in portions. The resulting solution was stirred for another 5 hr at room temperature and was subsequently concentrated. The resulting residue was applied onto a silica gel column with ethyl acetate/petroleum ether (1:5). This resulted in 26.9 g (95.0%) of 2,6-dibromo-4-chlorobenzenamine as brown solid LCMS of 2,6-dibromo-4-chlorobenzenamine (Method B): 286 [M+H]⁺, retention time 1.205 min.

### 2. Synthesis of 4-chloro-2,6-di(prop-1-en-2-yl)benzenamine

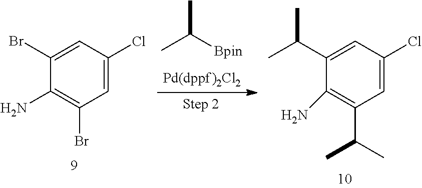

Into a 500-mL round-bottom flask, was placed 2,6-dibromo-4-chlorobenzenamine (5.7 g, 19.9 mmol, 1.0 equiv) in dioxane (150 mL) and water (15 mL). 4,4,5,5-tetramethyl-2-(prop-1-en-2-yl)-1,3,2-dioxaborolane (10.1 g, 60.0 mmol, 3.0 equiv), Cs₂CO₃ (19.6 g, 60.0 mmol, 3.0 equiv) and Pd(dppf)Cl₂ (1.5 g, 2.00 mmol, 0.03 equiv) were added to the solution. The resulting solution was stirred for 15 h at 90° C. using an oil bath. The resulting mixture was concentrated under vacuum. The residue obtained was applied onto a silica gel column, which was eluted with ethyl acetate/petroleum ether (1:3). This resulted in 3.6 g (88.0%) of 4-chloro-2,6-di(prop-1-en-2-yl)benzenamine as light yellow oil.

LCMS of 4-chloro-2,6-di(prop-1-en-2-yl)benzenamine (Method B): 208[M+H]⁺, retention time 1.205 min.

### 3. Synthesis of 4-chloro-2,6-diisopropylbenzenamine

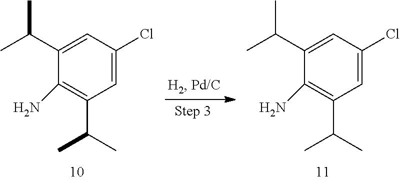

Into a 250-mL round-bottom flask, was placed 4-chloro-2,6-di(prop-1-en-2-yl)benzenamine (3.6 g, 17.2 mmol, 1.0 equiv) in methanol (50 mL). Pd/C (300 mg, 5%) was added to the solution in one portion under N₂ atmosphere. The resulting solution was stirred for 1 overnight at room temperature under H₂ atmosphere. The solids were filtered out. The filtrate was concentrated under vacuum. This resulted in 3.4 g (95%) of 4-chloro-2,6-diisopropylaniline as a light yellow solid.

LCMS of 4-chloro-2,6-diisopropylaniline (Method B): 212 [M+H]⁺, retention time 1.245 min.

### 4. Synthesis of 2-bromo-5-chloro-1,3-diisopropylbenzene

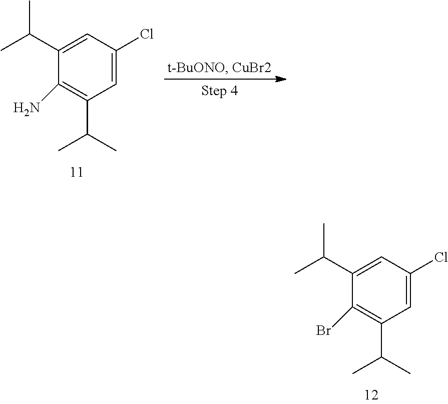

Into a 250 mL round-bottom flask, was placed a solution of 4-chloro-2,6-diisopropylbenzenamine (3.4 g, 16.0 mmol, 1 equiv) in MeCN(100 mL). CuBr₂ (7.1 g, 32.0 mmol, 2 equiv) was added to the solution, after which t-BuONO (3.3 g, 32.0 mmol, 2 equiv) was added dropwise at 0° C. The resulting solution was stirred for 30 min at room temperature and then stirred for 2 h at 70° C. The resulting mixture was concentrated in vacuo and purified with column chromatography [EtOAc/PE (1:10)] to give 2-bromo-5-chloro-1,3-diisopropylbenzene (2.4 g, 55.2%) as white solid.

### 5. Synthesis of tert-butyl 2-(4-chloro-2,6-diisopropylphenyl)acetate

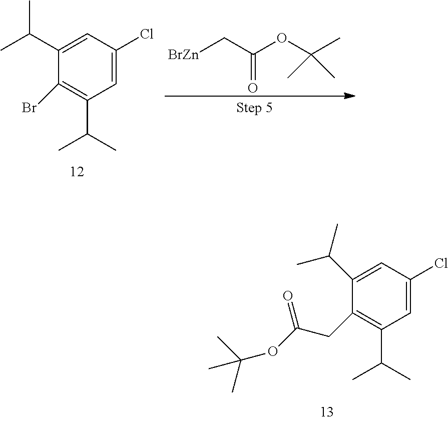

Into a 100 mL round-bottom flask, was placed a solution of 2-bromo-5-chloro-1,3-diisopropylbenzene (2.4 g, 8.7 mmol, 1 equiv) in THE (100 mL). Pd₂(dba)₃ (824.4 mg, 0.9 mmol, 0.1 equiv) and (2-tert-butoxy-2-oxoethyl)zinc(II) bromide (3.4 g, 13.1 mmol, 1.5 equiv) were added to the solution under N2 atmosphere. The resulting solution was stirred for 2 h at 70° C. The resulting mixture was concentrated in vacuo and purified with silica gel column chromatography [eluted with EtOAc/PE (1:10)] to give tert-butyl 2-(4-chloro-2,6-diisopropylphenyl)acetate (1.2 g, 44.4%) as white solid.

### 6. Synthesis of tert-butyl 2-[4-[(cyclopentyloxy)methyl]-2,6-bis(propan-2-yl)phenyl]acetate

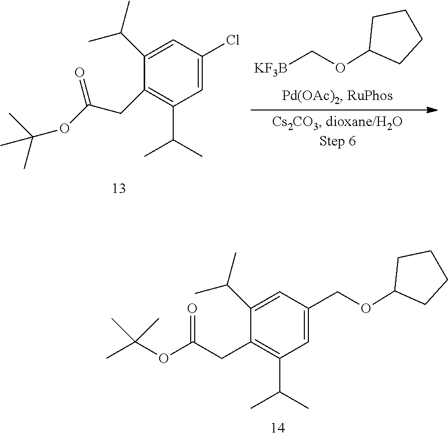

Into a 250 mL round-bottom flask were added tert-butyl 2-[4-chloro-2,6-bis(propan-2-yl)phenyl]acetate (1.2 g, 3.86 mmol, 1 equiv) and Cs₂CO₃ (3773.2 mg, 11.58 mmol, 3 equiv) at room temperature. To a stirred mixture of tert-butyl 2-[4-chloro-2,6-bis(propan-2-yl)phenyl]acetate (1.2 g, 3.86 mmol, 1 equiv) and Cs₂CO₃ (3773.2 mg, 11.58 mmol, 3 equiv) in dioxane (100 mL):H₂O (10 mL) was added Pd(OAc)₂ (173.3 mg, 0.77 mmol, 0.2 equiv), RuPhos (720.5 mg, 1.54 mmol, 0.4 equiv) and [(cyclopentyloxy)methyl] trifluoroborate potassium salt (1296.8 mg, 7.72 mmol, 2 equiv) in one portion at room temperature under nitrogen atmosphere. The resulting mixture was stirred overnight at 100 degrees C. under nitrogen atmosphere. The resulting mixture was concentrated under reduced pressure. The residue was purified by Prep-TLC (PE/EtOAc 15:1) to afford tert-butyl 2-[4-[(cyclopentyloxy)methyl]-2,6-bis(propan-2-yl)phenyl]acetate (875 mg, 60.5%) as a yellow oil.

¹H NMR (400 MHz, Chloroform-d) δ 7.13 (s, 2H), 4.48 (s, 2H), 4.14-3.95 (m, 1H), 3.72 (s, 2H), 3.21 (m, 2H), 1.91-1.74 (m, 6H), 1.63-1.52 (m, 2H), 1.46 (s, 9H), 1.26 (d, J=6.8 Hz, 12H).

### 7. Synthesis of 2-[4-[(cyclopentyloxy)methyl]-2,6-bis(propan-2-yl)phenyl]acetic acid

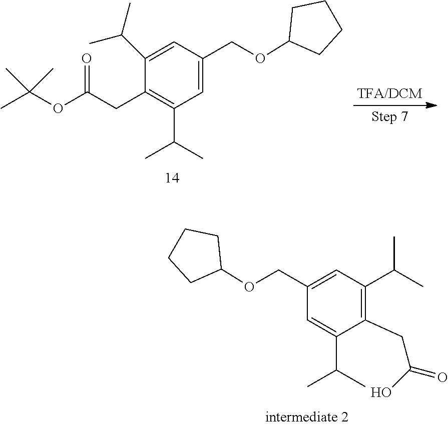 [SMILES: CC(C)c1cc(COC2CCCC2)cc(C(C)C)c1CC(=O)OC(C)(C)C ; CC(C)c1cc(COC2CCCC2)cc(C(C)C)c1CC(=O)O]

Into a 50 mL round-bottom flask were added tert-butyl 2-[4-[(cyclopentyloxy)methyl]-2,6-bis(propan-2-yl)phenyl]acetate) and DCM (5 mL) at room temperature. To a stirred solution of tert-butyl 2-[4-[(cyclopentyloxy)methyl]-2,6-bis(propan-2-yl)phenyl]acetate (875 mg, 2.34 mmol, 1 equiv) in DCM (5 mL) was added TFA (5 mL, 67.32 mmol, 28.82 equiv) dropwise at room temperature. The resulting mixture was concentrated under reduced pressure. The obtained residue was purified by reverse flash chromatography under the following conditions (column, C18 silica gel; mobile phase, MeCN in water, 50% to 90% gradient in 10 min; detector, UV 254 nm) to afford 2-[4-[(cyclopentyloxy)methyl]-2,6-bis(propan-2-yl)phenyl]acetic acid(530 mg) as a white solid.

LCMS of 2-[4-[(cyclopentyloxy)methyl]-2,6-bis(propan-2-yl)phenyl]acetic acid (Method B): 317 [M−H]⁻, retention time 1.245 min.

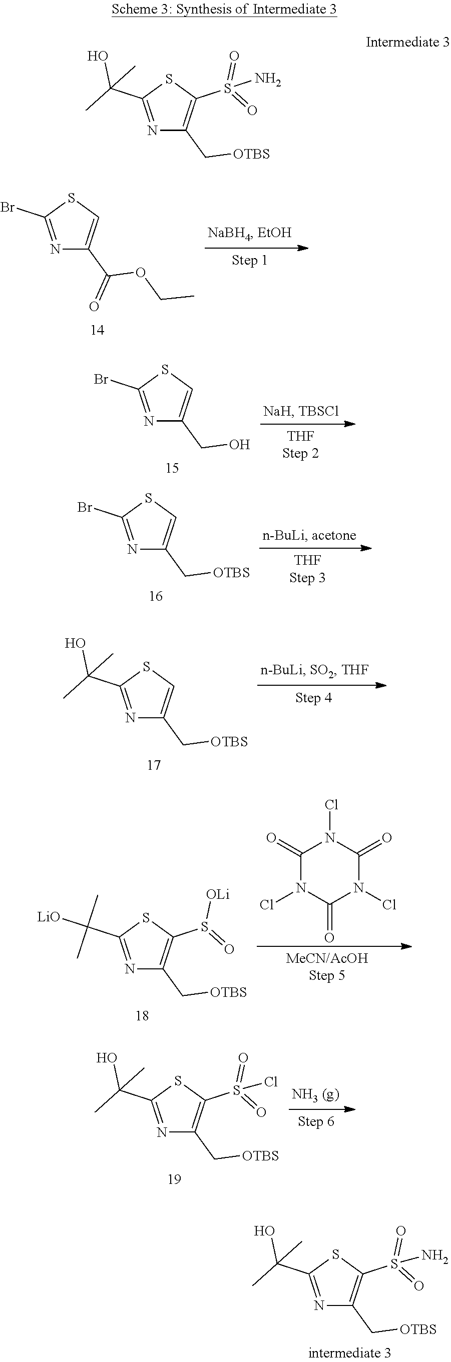

### 1. Synthesis of (2-bromo-1,3-thiazol-4-yl)methanol

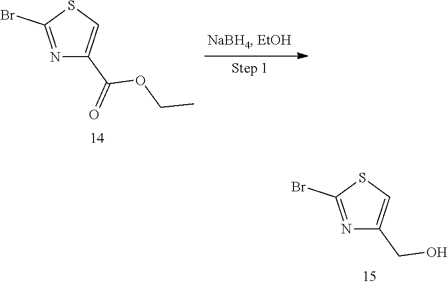

Into a 1 L round-bottom flask, was placed a solution of ethyl 2-bromo-1,3-thiazole-4-carboxylate (50 g, 211.79 mmol, 1 equiv) in EtOH (500 mL). NaBH₄ (16.0 g, 423.59 mmol, 2 equiv) was added to the solution in portions at 0° C. The resulting solution was stirred for 3 hr at room temperature. The reaction was then quenched by the addition of 1 L of ice-water. The resulting solution was extracted with 3×500 ml of ethyl acetate; the combined organic layers were dried over NaSO₄ and concentrated under vacuum. This resulted in 35 g (85.1%) of (2-bromo-1,3-thiazol-4-yl)methanol as yellow oil.

LCMS of (2-bromo-1,3-thiazol-4-yl)methanol (Method A): 194.0, 196.0 [M+H]⁺, retention time 0.581 min.

### 2. Synthesis of 2-bromo-4-[[(tert-butyldimethylsilyl)oxy]methyl]-1,3-thiazole

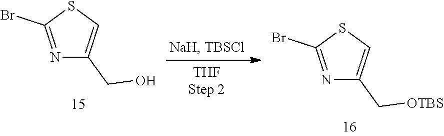 [SMILES: OCc1csc(Br)n1 ; CC(C)(C)[Si](C)(C)OCc1csc(Br)n1]

Into a 1-L round-bottom flask, was placed a solution of (2-bromo-1,3-thiazol-4-yl)methanol (35 g, 80.37 mmol, 1 equiv) in THE (400 mL). NaH (10.8 g, 70.86 mmol, 1.5 equiv, 60%) was added to the mixture in portions at 0° C. The mixture was stirred at 0° C. for another 1 h, after which TBSCl (43.5 g, 88.59 mmol, 1.6 equiv) was added to the mixture in portions at 0° C. The resulting solution was stirred for 2 hr at room temperature. The reaction was then quenched by the addition of 300 mL of ice-water. The resulting solution was extracted with 3×300 ml of ethyl acetate; the combined organic phase was dried over NaSO₄ and concentrated. The residue was applied onto a silica gel column with ethyl acetate/petroleum ether (1:30). This resulted in 30.0 g (53.9%) of 2-bromo-4-[[(tert-butyldimethylsilyl)oxy]methyl]-1,3-thiazole as yellow oil.

H-NMR of 2-bromo-4-[[(tert-butyldimethylsilyl)oxy]methyl]-1,3-thiazole: (CDCl₃, 300 MHz, ppm): δ 7.12 (t, J=1.5 Hz, 1H), 4.81 (d, J=1.5 Hz, 2H), 0.93 (s, 9H), 0.10 (s, 6H).

### 3. Synthesis of 2-(4-[[tert-butyldimethylsilyl)oxy]methyl]-1,3-thiazol-2-yl)propan-2-ol

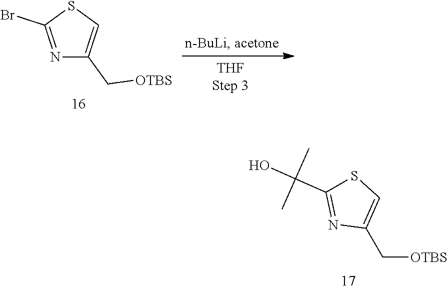 [SMILES: CC(C)(C)[Si](C)(C)OCc1csc(Br)n1 ; CC(C)(O)c1nc(CO[Si](C)(C)C(C)(C)C)cs1]

Into a 500-mL round-bottom flask purged and maintained with an inert atmosphere of nitrogen, was placed a solution of 2-bromo-4-[[(tert-butyldimethylsilyl)oxy]methyl]-1,3-thiazole (15.0 g, 48.65 mmol, 1 equiv) in THE (150 mL). n-BuLi (23.4 mL, 58.38 mmol, 2.5 M, 1.2 equiv) was added to the mixture in dropwise at −78° C. and the mixture stirred for 30 min at −78° C. Then propan-2-one (3.4 g, 58.38 mmol, 1.2 equiv) was added to the mixture dropwise at −78° C. The resulting mixture was stirred for another 1 h at room temperature. The reaction was then quenched by the addition of 200 mL of water. The resulting solution was extracted with 3×300 ml of ethyl acetate; the combined organic phase was dried over NaSO₄ and concentrated. The residue was applied onto a silica gel column with ethyl acetate/petroleum ether (1:10). This resulted in 12 g (85.7%) of 2-(4-[[(tert-butyldimethylsilyl)oxy]methyl]-1,3-thiazol-2-yl)propan-2-ol as yellow oil.

LC-MS of 2-(4-[[(tert-butyldimethylsilyl)oxy]methyl]-1,3-thiazol-2-yl)propan-2-ol (Method B): 288.2 [M+H]+, retention time 1.29 min.

### 4. Synthesis of 4-[[(tert-butyldimethylsilyl)oxy]methyl]-2-(2-hydroxypropan-2-yl)-1,3-thiazole-5-sulfonyl chloride

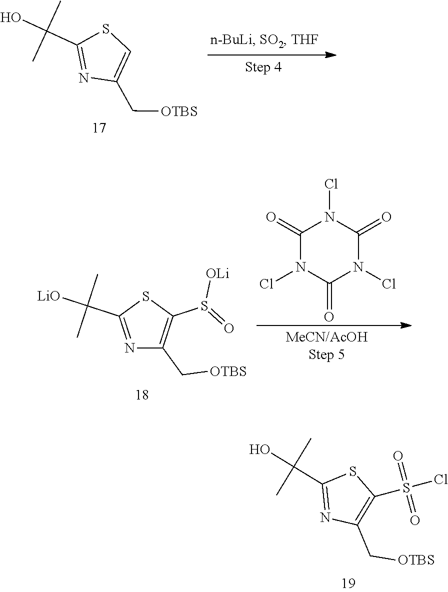

Into a 250-mL round-bottom flask purged with and maintained under an inert atmosphere of nitrogen, was placed a solution of 2-(4-[[(tert-butyldimethylsilyl)oxy]methyl]-1,3-thiazol-2-yl)propan-2-ol (10 g, 32.43 mmol, 1 equiv) in THE (100 mL), n-BuLi (39 mL, 97.7 mmol, 2.5 M, 3 equiv) was added to the mixture at −78° C. and stirred for another 30 min at −78° C. Then SO₂ was bubbled for 30 min and the mixture stirred for another 2 h at room temperature. The resulting mixture was concentrated. Then the residue obtained was dissolved in MeCN/AcOH (200 mL/10 mL). 1,3,5-trichloro-1,3,5-triazinane-2,4,6-trione (15.1 g, 64.86 mmol, 2 equiv) was added to the mixture in portions at 0° C., and the reaction was stirred for another 30 min at 0° C. The resulting mixture was concentrated at 0° C. This resulted in 12.5 g (92.9%) of 4-[[(tert-butyldimethylsilyl)oxy]methyl]-2-(2-hydroxypropan-2-yl)-1,3-thiazole-5-sulfonyl chloride as a yellow solid, which was used directly for the next step.

LC-MS of 4-[[(tert-butyldimethylsilyl)oxy]methyl]-2-(2-hydroxypropan-2-yl)-1,3-thiazole-5-sulfonyl chloride (Method B): 386.1 [M+H]+, retention time 1.456 min.

### 5. Synthesis of 4-[[(tert-butyldimethylsilyl)oxy]methyl]-2-(2-hydroxypropan-2-yl)-1,3-thiazole-5-sulfonamide

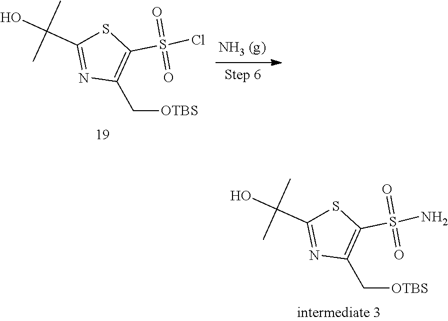 [SMILES: CC(C)(O)c1nc(CO[Si](C)(C)C(C)(C)C)c(S(=O)(=O)Cl)s1 ; CC(C)(O)c1nc(CO[Si](C)(C)C(C)(C)C)c(S(N)(=O)=O)s1]

Into a 250-mL round-bottom flask, was placed a solution of 4-[[tert-butyldimethylsilyl)oxy]methyl]-2-(2-hydroxypropan-2-yl)-1,3-thiazole-5-sulfonyl chloride (12.5 g, 32.38 mmol, 1 equiv) in DCM (130 mL), and NH3 was bubbled for 10 min. The resulting solution was stirred for another 1 hr at room temperature. The resulting mixture was concentrated. The residue was applied onto a silica gel column with ethyl acetate/petroleum ether (1:5). This resulted in 5.8 g (49.1%) of 4-[[(tert-butyldimethylsilyl)oxy]methyl]-2-(2-hydroxypropan-2-yl)-1,3-thiazole-5-sulfonamide as yellow oil.

LC-MS of 4-[[(tert-butyldimethylsilyl)oxy]methyl]-2-(2-hydroxypropan-2-yl)-1,3-thiazole-5-sulfonamide (Method B): 367.1 [M+H]+, retention time 1.184 min.

H-NMR-4-[[(tert-butyldimethylsilyl)oxy]methyl]-2-(2-hydroxypropan-2-yl)-1,3-thiazole-5-sulfonamide: (CD₃OD-d4, 400 MHz, ppm): δ 4.99 (s, 2H), 1.59 (s, 6H), 0.92 (s, 9H), 0.12 (s, 6H).

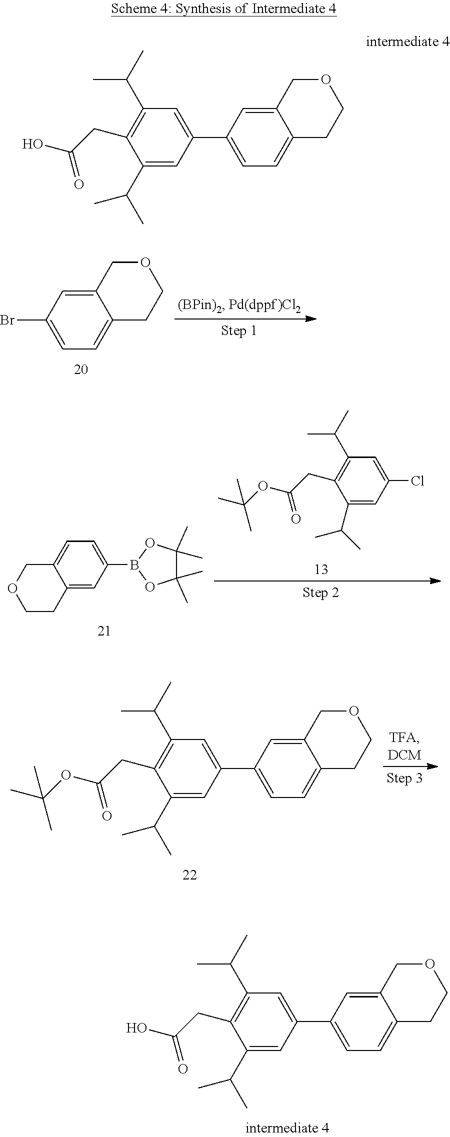

### 1. Synthesis of 2-(isochroman-6-yl)-4,4,5,5-tetramethyl-1,3,2-dioxaborolane

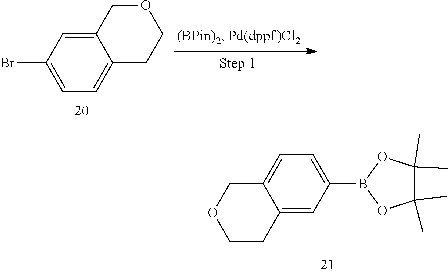 [SMILES: Brc1ccc2c(c1)COCC2 ; CC1(C)OB(c2ccc3c(c2)CCOC3)OC1(C)C]

Into a 500-mL round-bottom flask, was placed 7-bromoisochroman (8.48 g, 40.0 mmol, 1.0 equiv) in dioxane (200 mL)/water (20 mL). Pd(dppf)Cl₂ (5.8 g, 8.0 mmol, 0.2 equiv) and Cs₂CO₃ (26.1 g, 80.0 mmol, 2.0 equiv) (BPin)₂ (2.5 eq) were added to the solution. The resulting solution was stirred for 16 hr at 90 degrees C., after which it was concentrated and purified with SiO₂-gel column chromatography. This resulted in 4.6 g (44.2%) of 2-(isochroman-6-yl)-4,4,5,5-tetramethyl-1,3,2-dioxaborolane as a white solid.

### 2. Synthesis of tert-butyl 2-(4-(isochroman-7-yl)-2,6-diisopropylphenyl)acetate

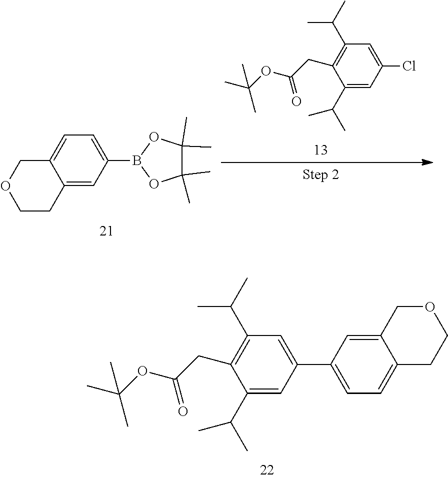 [SMILES: CC1(C)OB(c2ccc3c(c2)CCOC3)OC1(C)C ; CC(C)c1cc(Cl)cc(C(C)C)c1CC(=O)OC(C)(C)C ; CC(C)c1cc(-c2ccc3c(c2)COCC3)cc(C(C)C)c1CC(=O)OC(C)(C)C]

Into a 500-mL round-bottom flask, was placed 2-(isochroman-6-yl)-4,4,5,5-tetramethyl-1,3,2-dioxaborolane (4.6 g, 17.6 mmol, 1.0 equiv) and tert-butyl 2-(4-chloro-2,6-diisopropylphenyl)acetate (5.5 g, 17.6 mmol, 1.0 equiv) in dioxane (200 mL)/water (20 mL). Pd(dppf)Cl₂ (2.5 g, 3.5 mmol, 0.2 equiv) and Cs₂CO₃ (11.4 g, 35.0 mmol, 2.0 equiv) were added to the solution. The resulting solution was stirred for 16 hr at 90 degrees C. The resulting mixture was concentrated and purified with SiO₂-gel column chromatography. This resulted in 5.9 g (82.2%) of tert-butyl 2-(4-(isochroman-7-yl)-2,6-diisopropylphenyl)acetate as a white solid.

### 3. Synthesis of 2-(4-(isochroman-7-yl)-2,6-diisopropylphenyl)acetic acid

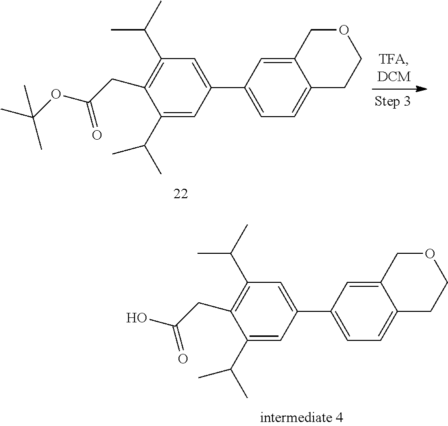 [SMILES: CC(C)c1cc(-c2ccc3c(c2)COCC3)cc(C(C)C)c1CC(=O)OC(C)(C)C ; CC(C)c1cc(-c2ccc3c(c2)COCC3)cc(C(C)C)c1CC(=O)O]

Into a 50-mL round-bottom flask was placed tert-butyl 2-(4-(isochroman-7-yl)-2,6-diisopropylphenyl)acetate (2.3 g, 5.6 mmol), DCM (10 mL), and TFA (10 mL). The resulting solution was stirred for 3 h at RT and was then concentrated under vacuum. The crude product was further dissolved in 100 mL of NaOH (4 N) and extracted with 3×50 mL of DCM to remove impurities. The pH value of aqueous phase was adjusted to 2 with HCl (4 N) and the aqueous phase was then extracted with 3×100 mL of DCM. The organic layers were combined, dried over anhydrous Na₂SO₄, and concentrated under vacuum. This resulted in 1.2 g (85%) of 2-(4-(isochroman-7-yl)-2,6-diisopropylphenyl)acetic acid as a light yellow solid.

LCMS of 2-(4-(isochroman-7-yl)-2,6-diisopropylphenyl)acetic acid (Method B): 351 [M−H]⁻, retention time 0.417 min.

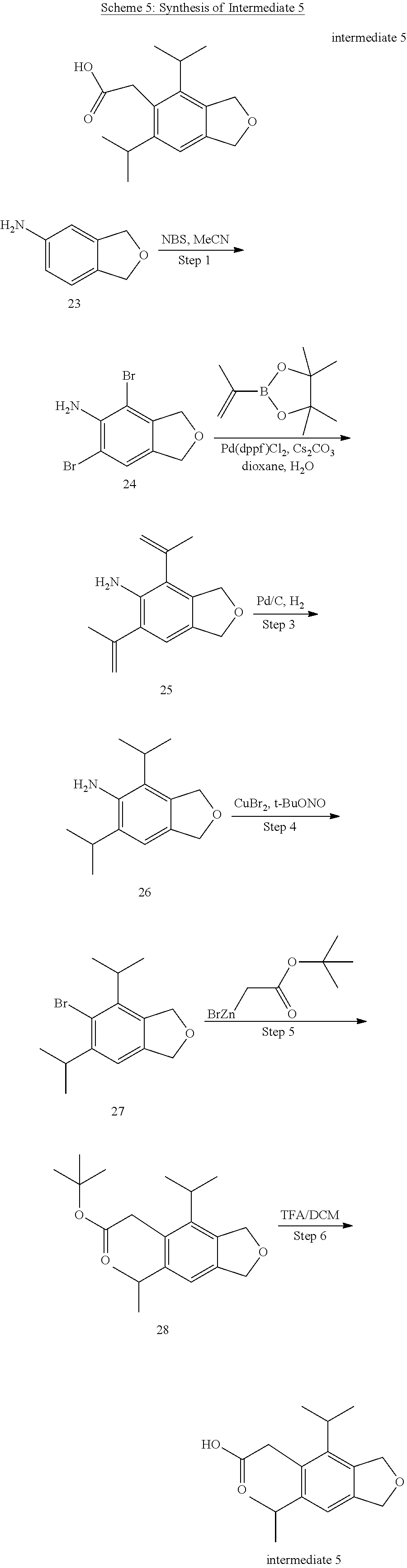

### 1. Synthesis of 4,6-dibromo-1,3-dihydroisobenzofuran-5-amine

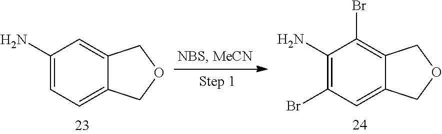 [SMILES: Nc1ccc2c(c1)COC2 ; Nc1c(Br)cc2c(c1Br)COC2]

Into a 250-mL round-bottom flask, was placed a solution of 1,3-dihydroisobenzofuran-5-amine (13.5 g, 100 mmol, 1 equiv) in MeCN (200 mL). NBS (44.5 g, 250 mmol, 2.5 equiv) was added to the solution in portions. The resulting solution was stirred for another 5 hr at room temperature. The resulting mixture was concentrated. The residue was applied onto a silica gel column with ethyl acetate/petroleum ether (1:5). This resulted in 26.3 g (91.0%) of 4,6-dibromo-1,3-dihydroisobenzofuran-5-amine as brown solid.

LCMS of 4,6-dibromo-1,3-dihydroisobenzofuran-5-amine (Method E): 291.9, 293.9, 295.9 [M+H]⁺, retention time 1.178 min.

### 2. Synthesis of 4,6-di(prop-1-en-2-yl)-1,3-dihydroisobenzofuran-5-amine

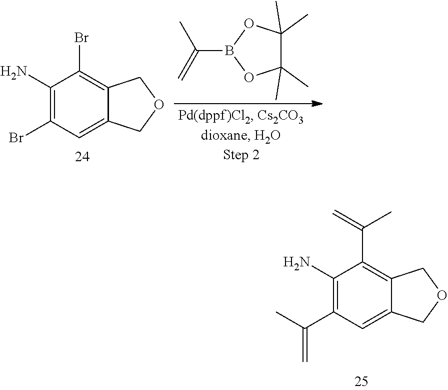

Into a 500-mL round-bottom flask, was placed 4,6-dibromo-1,3-dihydroisobenzofuran-5-amine (9.96 g, 34.0 mmol, 1.0 equiv) in dioxane (200 mL)/water (20 mL). Pd(dppf)Cl₂ (5.0 g, 6.8 mmol, 0.2 equiv) and Cs₂CO₃ (22.2 g, 68.0 mmol, 2.0 equiv) were added to the solution. The resulting solution was stirred for 16 hr at 90 degrees C. The resulting mixture was concentrated and purified with SiO₂-gel column. This resulted in 5.9 g (80.0%) of 4,6-di(prop-1-en-2-yl)-1,3-dihydroisobenzofuran-5-amine as a white solid.

LCMS of 4,6-di(prop-1-en-2-yl)-1,3-dihydroisobenzofuran-5-amine (Method E): 216.2 [M+H]⁺, retention time 1.208 min.

### 3. Synthesis of 4,6-diisopropyl-1,3-dihydroisobenzofuran-5-amine

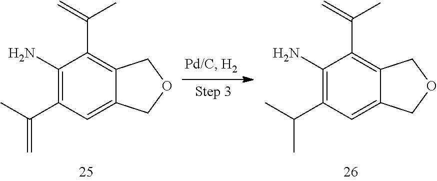 [SMILES: C=C(C)c1cc2c(c(C(=C)C)c1N)COC2 ; C=C(C)c1c(N)c(C(C)C)cc2c1COC2]

Into a 500 mL round-bottom flask were added 4,6-di(prop-1-en-2-yl)-1,3-dihydroisobenzofuran-5-amine (5.9 g, 27.5 mmol, 1 equiv) and isopropanol (250 mL) at room temperature. Pd/C (580 mg, 5.5 mmol, 0.20 equiv) was added to the solution at room temperature under nitrogen atmosphere. The resulting mixture was stirred overnight at room temperature under hydrogen atmosphere, and then filtered. The filtrate was concentrated under reduced pressure to afford 4,6-diisopropyl-1,3-dihydroisobenzofuran-5-amine (5.4 g, 90.0%) as a yellow solid.

LCMS of 4,6-diisopropyl-1,3-dihydroisobenzofuran-5-amine (Method E): 220.0 [M+H]⁺, retention time 1.132 min.

H-NMR-4,6-diisopropyl-1,3-dihydroisobenzofuran-5-amine: (DMSO-d6, 400 MHz, ppm): δ 6.83 (s, 1H), 5.01 (s, 2H), 4.82 (s, 2H), 3.19-3.11 (m, 1H), 3.01-2.98 (m, 1H), 1.18-1.14 (m, 12H).

### 4. Synthesis of 5-bromo-4,6-diisopropyl-1,3-dihydroisobenzofuran

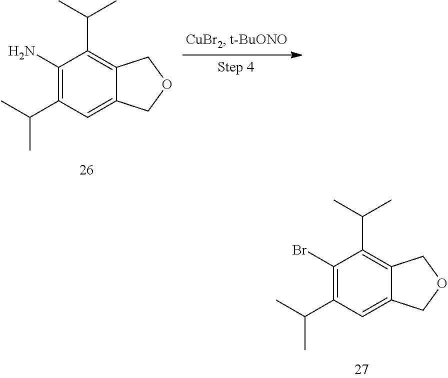 [SMILES: CC(C)c1cc2c(c(C(C)C)c1N)COC2 ; CC(C)c1cc2c(c(C(C)C)c1Br)COC2]

Into a 500 mL round-bottom flask, was placed a solution of 4,6-diisopropyl-1,3-dihydroisobenzofuran-5-amine (5.4 g, 24.7 mmol, 1 equiv) in MeCN (250 mL). CuBr₂ (11.0 g, 49.4 mmol, 2 equiv) was added to the solution, and then t-BuONO (5.1 g, 49.4 mmol, 2 equiv) was added to the solution in dropwise in 0° C. The resulting solution was stirred for 30 min at room temperature and then stirred for 2 h at 70° C. The resulting mixture was concentrated in vacuo and purified with silica gel column chromotography [eluted with EtOAc/PE (1:10)] to give 5-bromo-4,6-diisopropyl-1,3-dihydroisobenzofuran (3.8 g, 55.1%) as white solid.

H-NMR-5-bromo-4,6-diisopropyl-1,3-dihydroisobenzofuran: (DMSO-d6, 400 MHz, ppm): δ 7.20 (s, 1H), 5.13 (s, 2H), 4.91 (s, 2H), 3.46-3.39 (m, 2H), 1.22-1.18 (m, 12H).

### 5. Synthesis of tert-butyl 2-(4,6-diisopropyl-1,3-dihydroisobenzofuran-5-yl)acetate

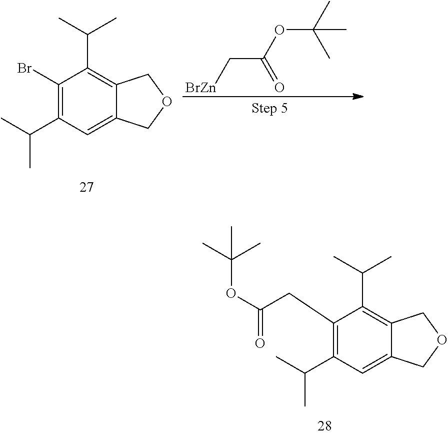

Into a 500 mL round-bottom flask, was placed a solution of 5-bromo-4,6-diisopropyl-1,3-dihydroisobenzofuran (3.8 g, 13.6 mmol, 1 equiv) in THE (250 mL), Pd₂(dba)₃ (1.3 g, 1.4 mmol, 0.1 equiv) and (2-tert-butoxy-2-oxoethyl)zinc(II) bromide (5.3 g, 20.4 mmol, 1.5 equiv) were added to the solution under N2 atmosphere. The resulting solution was stirred for 2 h at 70° C. The resulting mixture was concentrated under vacuo, purified with silica gel column, eluted with EtOAc/PE (1:10) to give tert-butyl 2-(4,6-diisopropyl-1,3-dihydroisobenzofuran-5-yl)acetate (2.4 g, 55.3%) as white solid.

H-NMR—tert-butyl 2-(4,6-diisopropyl-1,3-dihydroisobenzofuran-5-yl)acetate: (DMSO-d6, 400 MHz, ppm): δ 7.11 (s, 1H), 5.09 (s, 2H), 4.91 (s, 2H), 3.70 (s, 2H), 3.28-3.21 (m, 1H), 3.17-3.10 (m, 1H), 1.40 (s, 9H), 1.21-1.13 (m, 12H).

### 6. Synthesis of 2-(4,6-diisopropyl-1,3-dihydroisobenzofuran-5-yl)acetic acid

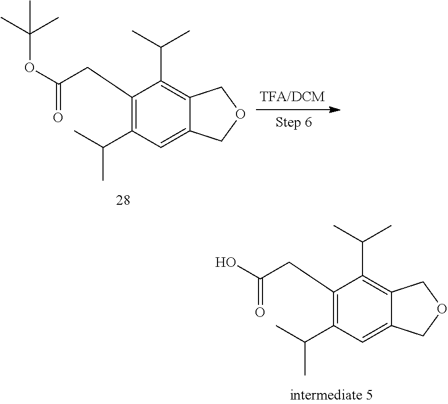 [SMILES: CC(C)c1cc2c(c(C(C)C)c1CC(=O)OC(C)(C)C)COC2 ; CC(C)c1cc2c(c(C(C)C)c1CC(=O)O)COC2]

Into a 500 mL round-bottom flask, was placed a solution of tert-butyl 2-(4,6-diisopropyl-1,3-dihydroisobenzofuran-5-yl)acetate (2.4 g, 7.5 mmol, 1 equiv) in DCM (100 mL). TFA (8.6 g, 75 mmol, 10 equiv) was added to the solution in dropwise at 0° C. The resulting solution was stirred for overnight at room temperature. The resulting mixture was concentrated in vacuo and purified with silica gel column chromatography [eluted with EtOAc/PE (1:10)] to give 2-(4,6-diisopropyl-1,3-dihydroisobenzofuran-5-yl)acetic acid (1.7 g, 87.5%) as yellow solid.

LCMS of 2-(4,6-diisopropyl-1,3-dihydroisobenzofuran-5-yl)acetic acid (Method E): 263.2 [M+H]⁺, retention time 1.131 min.

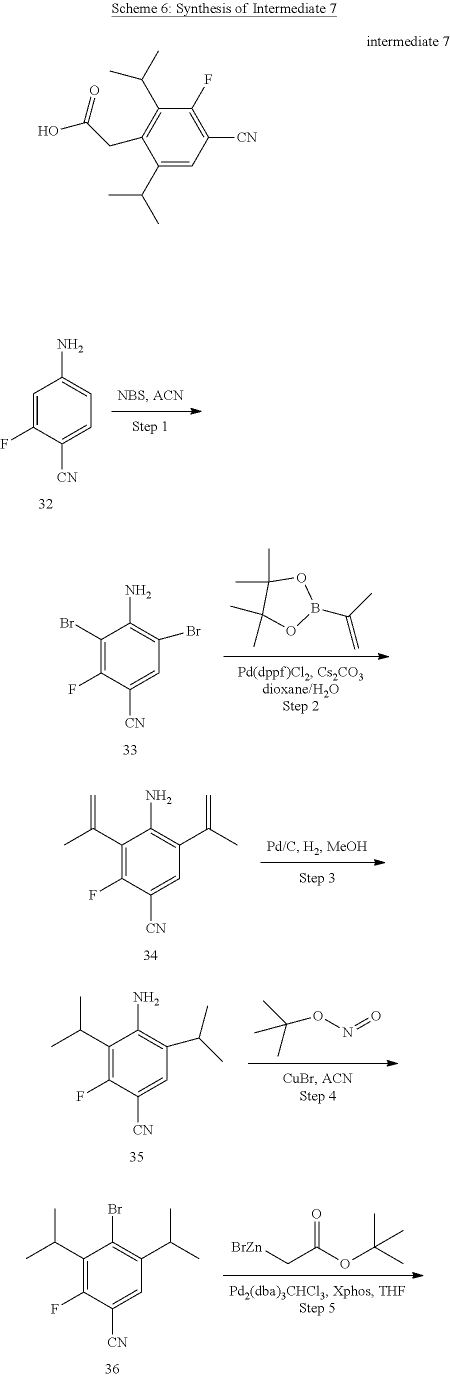

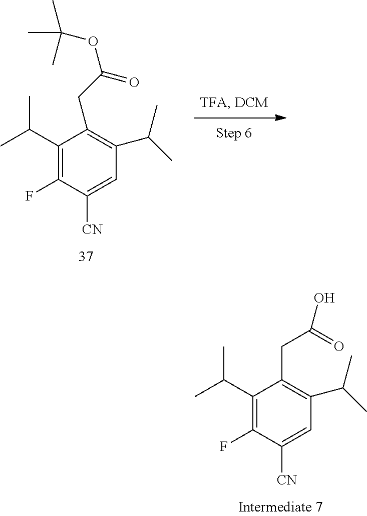 [SMILES: CC(C)c1cc(C#N)c(F)c(C(C)C)c1CC(=O)OC(C)(C)C ; CC(C)c1cc(C#N)c(F)c(C(C)C)c1CC(=O)O]

### 1. Synthesis of 4-amino-3,5-dibromo-2-fluorobenzonitrile

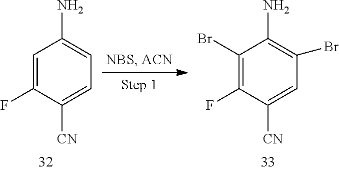 [SMILES: N#Cc1ccc(N)cc1F ; N#Cc1cc(Br)c(N)c(Br)c1F]

Into a 250-mL round-bottom flask, was placed a solution of 4-amino-2-fluorobenzonitrile (13.6 g, 100 mmol, 1 equiv) in MeCN (200 mL). NBS (44.5 g, 250 mmol, 2.5 equiv) was added to the solution in portions. The resulting solution was stirred for another 5 hr at room temperature. The resulting mixture was concentrated. The residue obtained was applied onto a silica gel column which was eluted with ethyl acetate/petroleum ether (1:5). This resulted in 26.4 g (91.0%) of 4-amino-3,5-dibromo-2-fluorobenzonitrile as brown solid.

LCMS of 4-amino-3,5-dibromo-2-fluorobenzonitrile (Method E): 293.9 [M−1+H]⁺, retention time 1.290 min.

### 2. Synthesis of 4-amino-2-fluoro-3,5-di(prop-1-en-2-yl)benzonitrile

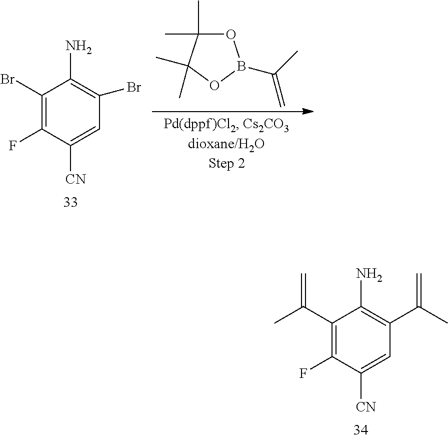

Into a 500-mL round-bottom flask, was placed 4-amino-3,5-dibromo-2-fluorobenzonitrile (10.0 g, 34.0 mmol, 1.0 equiv) in dioxane (200 mL)/water (20 mL). Pd(dppf)Cl₂ (5.0 g, 6.8 mmol, 0.2 equiv) and Cs₂CO₃ (22.2 g, 68.0 mmol, 2.0 equiv) were added to the solution. The resulting solution was stirred for 16 hr at 90 degrees C. The resulting mixture was concentrated and purified with SiO₂-gel column chromatography. This resulted in 5.9 g (81.0%) of 4-amino-2-fluoro-3,5-di(prop-1-en-2-yl)benzonitrile as a white solid.

LCMS of 4-amino-2-fluoro-3,5-di(prop-1-en-2-yl)benzonitrile (Method E): 217.2 [M+H]⁺, retention time 1.182 min.

### 3. Synthesis of 4-amino-2-fluoro-3,5-diisopropylbenzonitrile

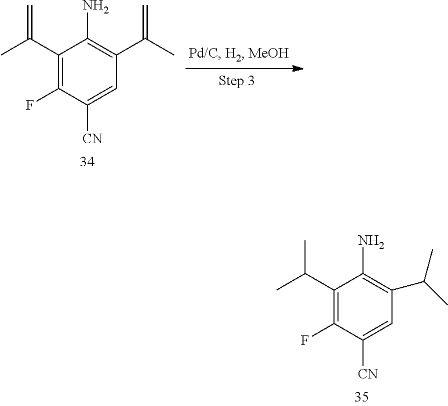 [SMILES: C=C(C)c1cc(C#N)c(F)c(C(=C)C)c1N ; CC(C)c1cc(C#N)c(F)c(C(C)C)c1N]

Into a 500 mL round-bottom flask were added 4-amino-2-fluoro-3,5-di(prop-1-en-2-yl)benzonitrile (5.9 g, 27.5 mmol, 1 equiv) and isopropanol(250 mL) at room temperature. Pd/C (580 mg, 5.5 mmol, 0.20 equiv) was added to the solution at room temperature under nitrogen atmosphere. The resulting mixture was stirred overnight at room temperature under hydrogen atmosphere, after which it was filtered. The filtrate was concentrated under reduced pressure to afforded 4-amino-2-fluoro-3,5-diisopropylbenzonitrile (5.1 g, 84.0%) as yellow solid.

1H NMR of 4-amino-2-fluoro-3,5-diisopropylbenzonitrile (300 MHz, CDCl3-d) 67.16 (d, J=6.8 Hz, 1H), 4.37 (s, 2H), 3.16-2.95 (m, 1H), 2.89-2.65 (m, 1H), 1.36 (dd, J=7.1, 1.8 Hz, 6H), 1.25 (d, J=6.8 Hz, 6H).

### 4. Synthesis of 4-bromo-2-fluoro-3,5-diisopropylbenzonitrile

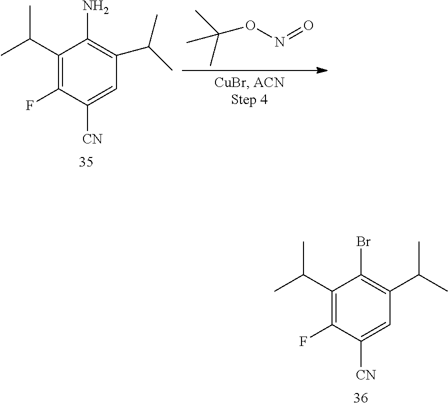

Into a 500 mL round-bottom flask, was placed a solution of 4-amino-2-fluoro-3,5-diisopropylbenzonitrile (5.1 g, 23.1 mmol, 1 equiv) in MeCN (250 mL). CuBr₂ (10.3 g, 46.2 mmol, 2 equiv) was added to the solution, and then t-BuONO (4.8 g, 46.2 mmol, 2 equiv) was added to the solution in dropwise in 0° C. The resulting solution was stirred for 30 min at room temperature and then stirred for 2 h at 70° C. The resulting mixture was concentrated in vacuo and purified with silica gel column chromatography [eluted with EtOAc/PE (1:10)] to give 4-bromo-2-fluoro-3,5-diisopropylbenzonitrile (3.6 g, 55.0%) as white solid.

1H NMR of 4-bromo-3,5-diisopropylbenzonitrile (400 MHz, Methanol-d4) δ 7.58 (d, J=6.6 Hz, 1H), 3.81 (m, 1H), 3.52 (m, 1H), 1.38 (dd, J=7.1, 1.9 Hz, 6H), 1.27 (d, J=6.8 Hz, 6H).

### 5. Synthesis of tert-butyl 2-(4-cyano-3-fluoro-2,6-diisopropylphenyl)acetate

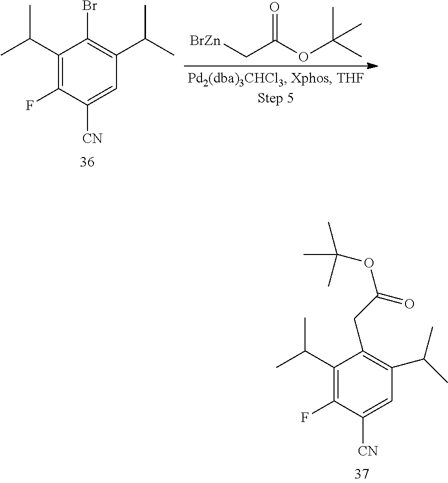

Into a 500 mL round-bottom flask, was placed a solution of 4-bromo-2-fluoro-3,5-diisopropylbenzonitrile (3.6 g, 12.7 mmol, 1 equiv) in THE (250 mL), Pd₂(dba)₃ (1.2 g, 1.3 mmol, 0.1 equiv) and (2-tert-butoxy-2-oxoethyl)zinc(II) bromide (5.0 g, 19.1 mmol, 1.5 equiv) were added to the solution under N2 atmosphere. The resulting solution was stirred for 2 h at 70° C. The resulting mixture was concentrated in vacuo and purified with silica gel column chromatography [eluted with EtOAc/PE (1:10)] to give tert-butyl 2-(4-cyano-3-fluoro-2,6-diisopropylphenyl)acetate (3.1 g, 78.0%) as white solid.

1H NMR (300 MHz, Methanol-d4) δ 7.52 (d, J=6.4 Hz, 1H), 3.86 (s, 2H), 3.31-3.16 (m, 2H), 1.46 (s, 9H) 1.35 (dd, J=7.1, 1.8 Hz, 6H), 1.24 (d, J=6.9 Hz, 6H).

### 6. Synthesis of 2-(4-cyano-3-fluoro-2,6-diisopropylphenyl)acetic acid

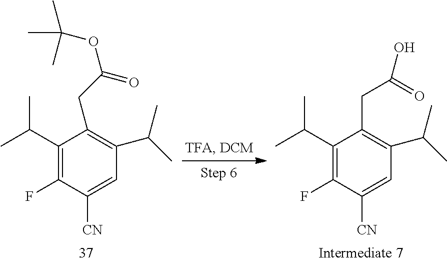 [SMILES: CC(C)c1cc(C#N)c(F)c(C(C)C)c1CC(=O)OC(C)(C)C ; CC(C)c1cc(C#N)c(F)c(C(C)C)c1CC(=O)O]

Into a 500 mL round-bottom flask, was placed a solution of tert-butyl 2-(4-cyano-3-fluoro-2,6-diisopropylphenyl)acetate (3.1 g, 9.9 mmol, 1 equiv) in DCM (100 mL). TFA (11.3 g, 99 mmol, 10 equiv) was added to the solution in dropwise at 0° C. The resulting solution was stirred overnight at room temperature. The resulting mixture was concentrated in vacuo and purified with silica gel column chromatography [eluted with EtOAc/PE (1:10)] to give 2-(4-cyano-3-fluoro-2,6-diisopropylphenyl)acetic acid (1.7 g, 65.7%) as white solid.

LCMS of 2-(4-cyano-3-fluoro-2,6-diisopropylphenyl)acetic acid (Method E): 264.2 [M+H]⁺, retention time 1.519 min.

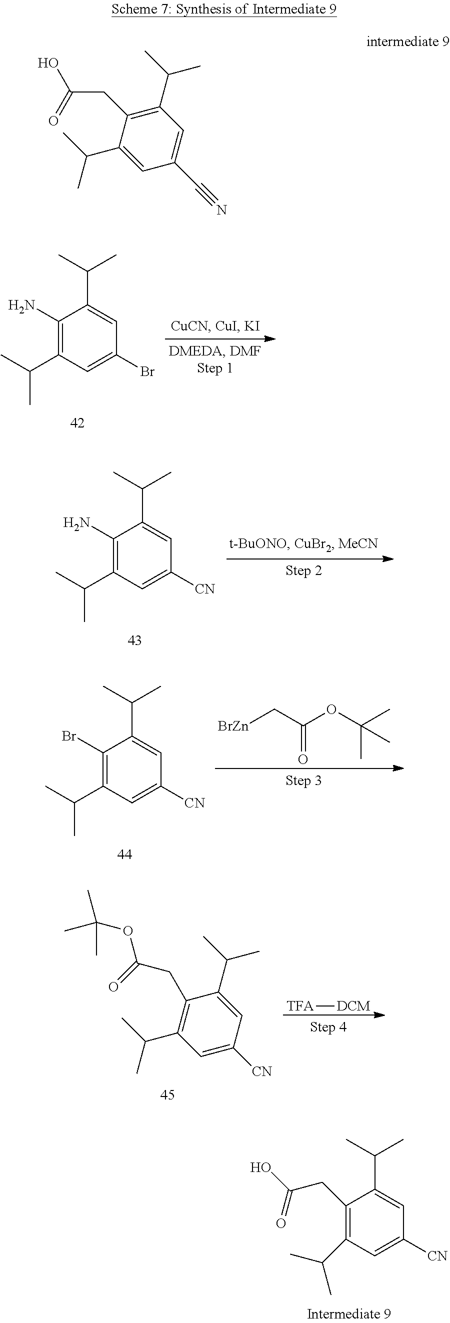

### 1. Synthesis of 4-Amino-3,5-diisopropylbenzonitrile

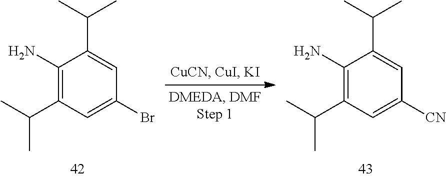

Into a 100-mL round-bottom flask purged with and maintained under nitrogen, was placed 4-bromo-2,6-diisopropylbenzenamine (commercially available, 5.1 g, 19.9 mmol), DMF (30 mL), CuCN (2.16 g, 23.9 mmol), CuI (380 mg, 2.00 mmol), KI (664 mg, 3.98 mmol), and DMEDA (2.0 mL). The resulting solution was stirred for 24 h at 100° C. and then was diluted with 30 mL of water. The solution was extracted with 3×30 mL of ethyl acetate and the organic layers combined and concentrated under vacuum. The residue was applied onto a silica gel column and eluted with ethyl acetate/petroleum ether (1:30 to 1:20). This resulted in 1.2 g (30%) of the title compound as a yellow solid. MS-ESI: 203.1 (M+1).

### 2. Synthesis of 4-bromo-3,5-diisopropylbenzonitrile

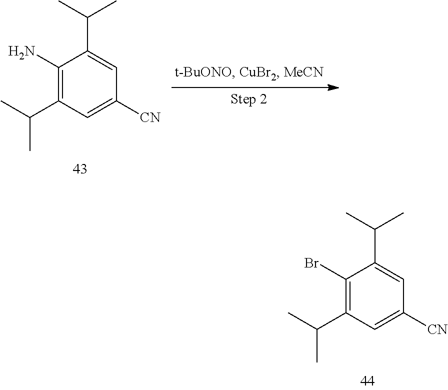

Into a 500 mL round-bottom flask, was placed a solution of 4-amino-3,5-diisopropylbenzonitrile (10.0 g, 49.5 mmol, 1 equiv) in MeCN(250 mL). CuBr₂ (22.1 g, 99 mmol, 2 equiv) was added to the solution, and then t-BuONO (10.2 g, 99 mmol, 2 equiv) was added to the solution in dropwise in 0° C. The resulting solution was stirred for 30 min at room temperature and then stirred for 2 h at 70° C. The resulting mixture was concentrated in vacuo and purified with silica gel column chromatography [eluted with EtOAc/PE (1:10)] to give 4-bromo-3,5-diisopropylbenzonitrile (6.0 g, 45.2%) as white solid.

### 3. Synthesis of tert-butyl 2-(4-cyano-2,6-diisopropylphenyl)acetate

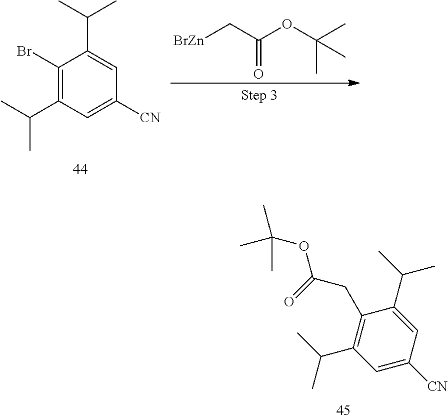

Into a 500 mL round-bottom flask, was placed a solution of 4-bromo-3,5-diisopropylbenzonitrile (6.0 g, 22.4 mmol, 1 equiv) in THE (250 mL), Pd₂(dba)₃ (2.0 g, 2.2 mmol, 0.1 equiv) and (2-tert-butoxy-2-oxoethyl)zinc(II) bromide (8.7 g, 33.6 mmol, 1.5 equiv) were added to the solution under N2 atmosphere. The resulting solution was stirred for 2 h at 70° C. The resulting mixture was concentrated under vacuo, purified with silica gel column, eluted with EtOAc/PE (1:10) to give tert-butyl 2-(4-cyano-2,6-diisopropylphenyl)acetate (4.2 g, 62.0%) as white solid.

1H NMR (400 MHz, DMSO-d6) δ 7.60 (s, 2H), 3.80 (s, 2H), 3.20-3.10 (m, 2H), 1.39 (s, 9H), 1.18 (d, J=6.8 Hz, 12H).

### 4. Synthesis of 2-(4-cyano-2,6-diisopropylphenyl)acetic acid

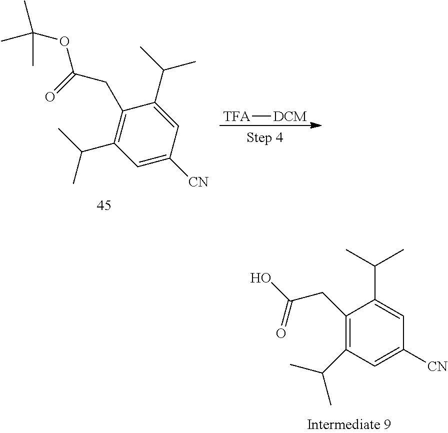

Into a 500 mL round-bottom flask, was placed a solution of tert-butyl 2-(4-cyano-2,6-diisopropylphenyl)acetate (4.2 g, 13.9 mmol, 1 equiv) in DCM (200 mL). TFA (15.8 g, 139 mmol, 10 equiv) was added to the solution dropwise at 0° C. The resulting solution was stirred overnight at room temperature. The resulting mixture was concentrated in vacuo and the purified with silica gel column chromatography [eluted with EtOAc/PE (1:10)] to give tert-butyl 2-(4-cyano-2,6-diisopropylphenyl)acetate (3.0 g, 88.2%) as white solid.

LCMS of tert-butyl 2-(4-cyano-2,6-diisopropylphenyl)acetate (Method: Kinetex EVO C18 100 A, 50*3.0 mm, 0.6 uL injection, 1.5 mL/min flowrate, 90-900 amu scan range, 254 nm UV detection. Mobile phase A: Water (0.1% FA) and Mobile Phase B: MeCN+0.05% FA. 5% MPB to 100% in 3.26 min, hold at 100% MPB for 0.80 min, 100% MPB to 5% in 0.02 min, then equilibration to 5% MPB for 0.4 min.): 489.4 [2M−H]⁺, retention time 2.253 min.

1H NMR (400 MHz, DMSO-d6) δ 12.58 (s, 1H), 7.58 (s, 2H), 3.80 (s, 2H), 3.17-3.11 (m, 2H), 1.17 (d, J=6.8 Hz, 12H).

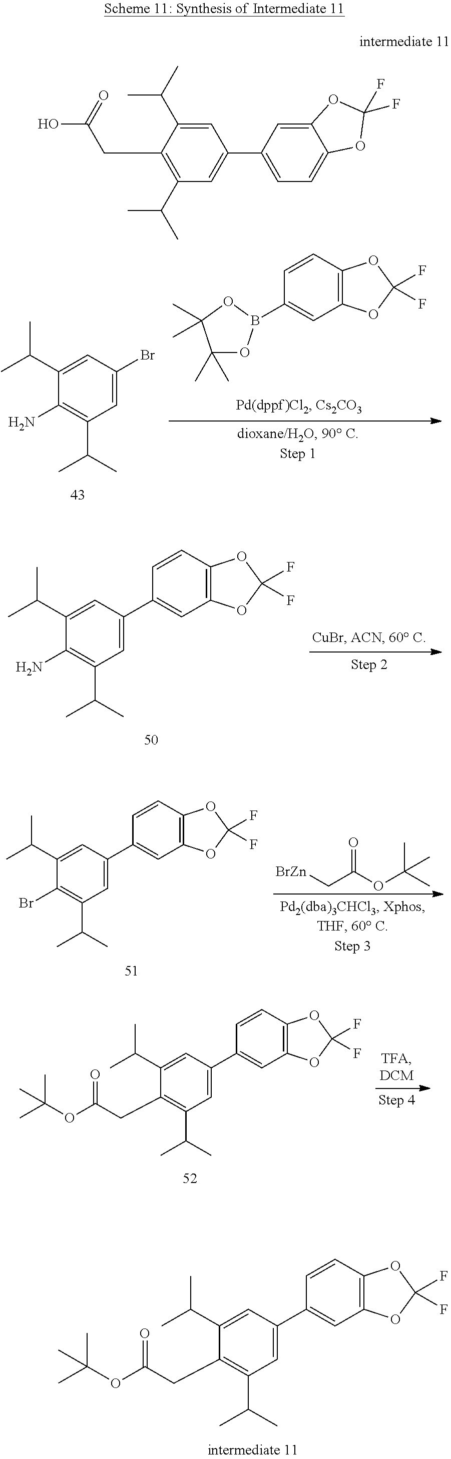

### 1. Synthesis of 4-(2,2-difluoro-2H-1,3-benzodioxol-5-yl)-2,6-bis(propan-2-yl)aniline

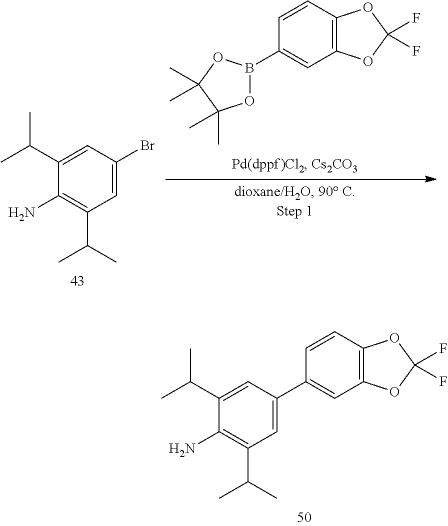 [SMILES: CC(C)c1cc(Br)cc(C(C)C)c1N ; CC1(C)OB(c2ccc3c(c2)OC(F)(F)O3)OC1(C)C ; CC(C)c1cc(-c2ccc3c(c2)OC(F)(F)O3)cc(C(C)C)c1N]

Into a 500-mL round-bottom flask purged with and maintained under an inert atmosphere of nitrogen, was placed 4-bromo-2,6-bis(propan-2-yl)aniline (7 g, 27.32 mmol, 1 equiv), dioxane (80 mL), H₂O (10 mL, 0.56 mmol, 0.02 equiv), Cs₂CO₃ (19.6 g, 60.11 mmol, 2.20 equiv), 2-(2,2-difluoro-2H-1,3-benzodioxol-5-yl)-4,4,5,5-tetramethyl-1,3,2-dioxaborolane (10.0 g, 35.52 mmol, 1.30 equiv), and Pd(dppf)Cl₂ (4.0 g, 5.5 mmol, 0.2 equiv). The resulting solution was stirred for 16 hr at 90° C. in an oil bath. The resulting mixture was concentrated. The residue was applied onto a silica gel column with ethyl acetate/hexane (1:1). This resulted in 6.1 g (66.97%) of 4-(2,2-difluoro-2H-1,3-benzodioxol-5-yl)-2,6-bis(propan-2-yl)aniline as a yellow solid.

LC-MS-4-(2,2-difluoro-2H-1,3-benzodioxol-5-yl)-2,6-bis(propan-2-yl)aniline (Method A): 334.1 [M+H]⁺, retention time 1.519 min.

### 2. Synthesis of 5-(4-bromo-3,5-diisopropylphenyl)-2,2-difluorobenzo[d][1,3]dioxole

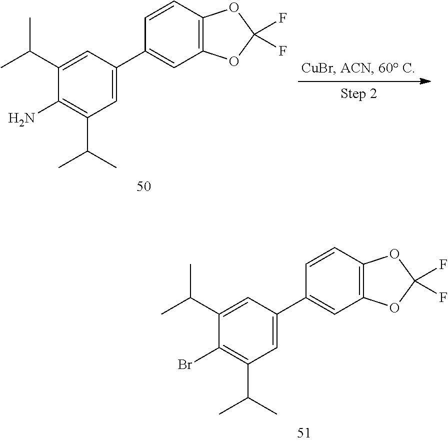 [SMILES: CC(C)c1cc(-c2ccc3c(c2)OC(F)(F)O3)cc(C(C)C)c1N ; CC(C)c1cc(-c2ccc3c(c2)OC(F)(F)O3)cc(C(C)C)c1Br]

Into a 250-mL round-bottom flask purged with and maintained under an inert atmosphere of nitrogen, was placed 4-(2,2-difluoro-2H-1,3-benzodioxol-5-yl)-2,6-bis(propan-2-yl)aniline (6.12 g, 18.36 mmol, 1 equiv) and ACN (80 mL). This was followed by the addition of CuBr (5266.8 mg, 36.72 mmol, 2.00 equiv). To this was added tert-Butyl nitrite (3786.0 mg, 36.71 mmol, 2 equiv). The resulting solution was stirred for 3 hr at 60° C. in an oil bath. The resulting mixture was concentrated. The residue was applied onto a silica gel column with ethyl acetate/petroleum ether (1:10). This resulted in 2.61 g (35.79%) of 5-[4-bromo-3,5-bis(propan-2-yl)phenyl]-2,2-difluoro-2H-1,3-benzodioxole as a yellow solid.

LC-MS-(4-bromo-3,5-diisopropylphenyl)-2,2-difluorobenzo[d][1,3]dioxole (Method G): retention time 0.878 min.

### 3. Synthesis of tert-butyl 2-[4-(2,2-difluoro-2H-1,3-benzodioxol-5-yl)-2,6-bis(propan-2-yl)phenyl]acetate

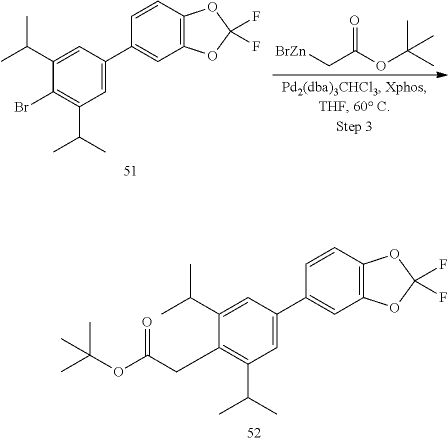

Into a 100-mL round-bottom flask purged and maintained with an inert atmosphere of nitrogen, was placed 5-[4-bromo-3,5-bis(propan-2-yl)phenyl]-2,2-difluoro-2H-1,3-benzodioxole (2.6 g, 6.54 mmol, 1 equiv), Pd₂(dba)₃CHCl₃ (677.5 mg, 0.65 mmol, 0.10 equiv), Xphos (624.0 mg, 1.31 mmol, 0.2 equiv), and THF (40 mL). The resulting solution was stirred for 20 min at RT. Then added tert-butyl 2-(bromozincio)acetate (5113.6 mg, 19.63 mmol, 3 equiv). The resulting solution was stirred for 2 hr at 60° C. in an oil bath. The resulting mixture was concentrated. The residue was applied onto a silica gel column with ethyl acetate/petroleum ether (1:20). This resulted in 2.1 g (74.19%) of tert-butyl 2-[4-(2,2-difluoro-2H-1,3-benzodioxol-5-yl)-2,6-bis(propan-2-yl)phenyl]acetate as yellow oil.

### 4. Synthesis of 2-[4-(2,2-difluoro-2H-1,3-benzodioxol-5-yl)-2,6-bis(propan-2-yl)phenyl]acetic acid

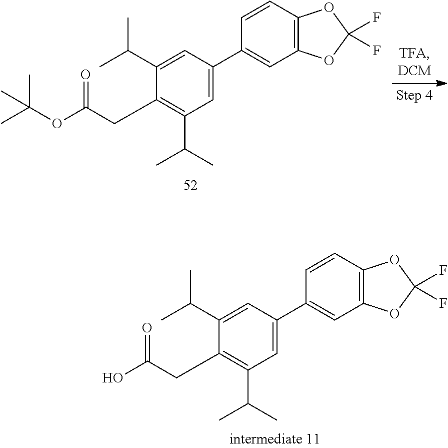 [SMILES: CC(C)c1cc(-c2ccc3c(c2)OC(F)(F)O3)cc(C(C)C)c1CC(=O)OC(C)(C)C ; CC(C)c1cc(-c2ccc3c(c2)OC(F)(F)O3)cc(C(C)C)c1CC(=O)O]

Into a 100-mL round-bottom flask, was placed tert-butyl 2-[4-(2,2-difluoro-2H-1,3-benzodioxol-5-yl)-2,6-bis(propan-2-yl)phenyl]acetate (2.1 g, 4.86 mmol, 1 equiv), DCM (20 mL), and TFA (10 mL, 134.63 mmol, 27.73 equiv). The resulting solution was stirred for 2 hr at room temperature. The resulting mixture was concentrated. The residue was applied onto a silica gel column which was eluted with ethyl acetate/petroleum ether (1:2). This resulted in 1.36 g (74.42%) of 2-[4-(2,2-difluoro-2H-1,3-benzodioxol-5-yl)-2,6-bis(propan-2-yl)phenyl]acetic acid as a yellow solid.

LC-MS-2-[4-(2,2-difluoro-2H-1,3-benzodioxol-5-yl)-2,6-bis(propan-2-yl)phenyl]acetic acid (Method G): 375.2 [M−H]⁺, retention time 1.050 min.

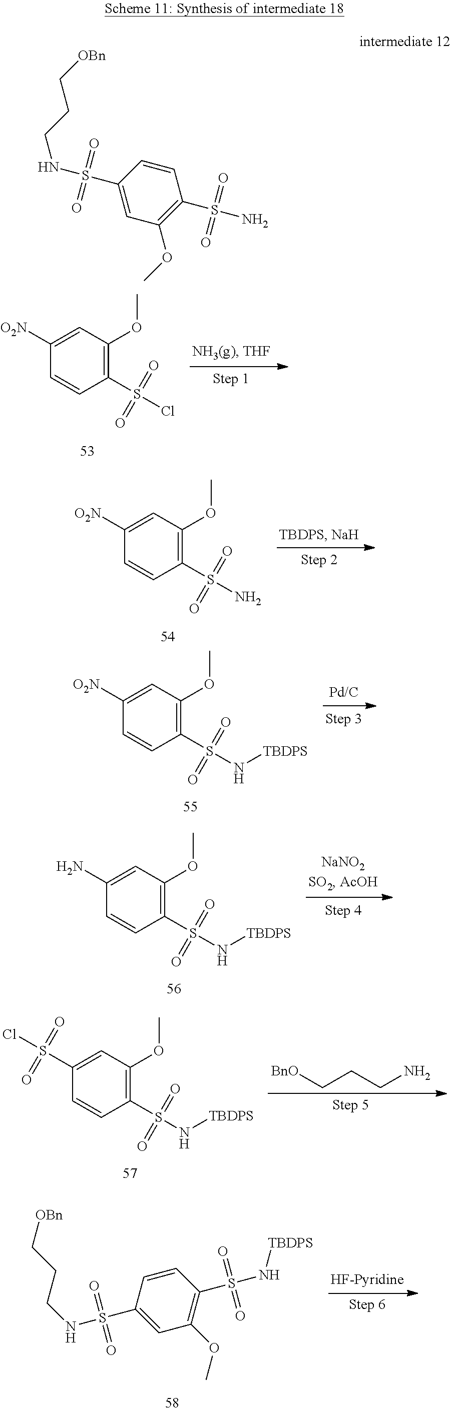

### 1. Synthesis of 2-methoxy-4-nitrobenzenesulfonamide

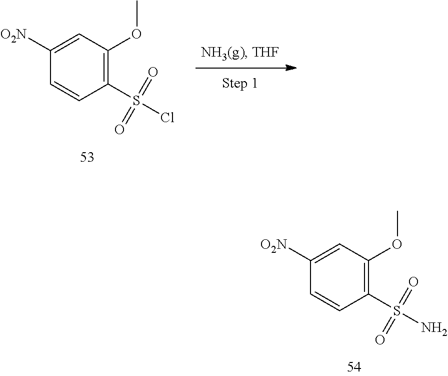

Into a 2 L round-bottom flask, was placed a solution of 2-methoxy-4-nitrobenzene-1-sulfonyl chloride (27.3 g, 109 mmol, 1 equiv) in THE (1 L). To the solution was bubbled NH3 (g) for 0.5 h at 0° C. The reaction was stirred at 0° C. for another 2 h, after which it was concentrated under reduced pressure to remove the solvent, diluted with water (400 mL), and extracted with EtOAc(200 mL*3). The combined organic phase was washed with water (200 mL*2) and brine (200 mL*1). The organic layer was dried over NaSO₄ and concentrated in vacuo to give 2-methoxy-4-nitrobenzenesulfonamide (23.2 g, 92.0%) as yellow solid.

1H NMR (300 MHz, DMSO-d6) δ 7.91 (d, J=8.3 Hz, 1H), 7.81-7.63 (m, 2H), 3.88 (s, 3H).

### 2. Synthesis of N-(tert-butyldiphenylsilyl)-2-methoxy-4-nitrobenzenesulfonamide

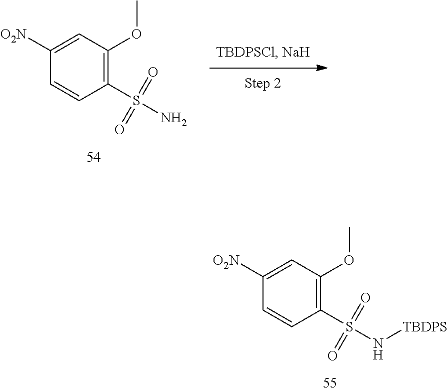

Into a 1 L round-bottom flask, was placed a solution of 2-methoxy-4-nitrobenzenesulfonamide (23.2 g, 100 mmol, 1 equiv) in THE (250 mL). NaH (8.0 g, 200.0 mmol, 2 equiv, 60%) was added to the solution in portions at 0° C. TBDPSCl (54.8 g, 200.0 mmol, 2 equiv) was added to the mixture at 0° C. The resulting solution was stirred overnight at room temperature. The reaction was then quenched by the addition of 1 L of ice-water. The resulting solution was extracted with 3×500 ml of ethyl acetate; the combined organic layers were dried over NaSO₄ and concentrated under vacuum. The residue was applied onto a silica gel column which was eluted with ethyl acetate/petroleum ether (1:5) to give N-(tert-butyldiphenylsilyl)-2-methoxy-4-nitrobenzenesulfonamide (28 g, 59.6%) as yellow solid.

1H NMR—N-(tert-butyldiphenylsilyl)-2-methoxy-4-nitrobenzenesulfonamide (300 MHz, CDCl3-d) δ 7.80 (d, J=2.1 Hz, 1H), 7.60-7.53 (m, 4H), 7.51 (dd, J=8.6, 2.0 Hz, 1H), 7.46-7.38 (m, 1H), 7.32-7.19 (m, 6H), 5.41 (s, 1H), 4.16 (s, 3H), 1.08 (s, 9H).

### 3. Synthesis of 4-amino-N-(tert-butyldiphenylsilyl)-2-methoxybenzenesulfonamide

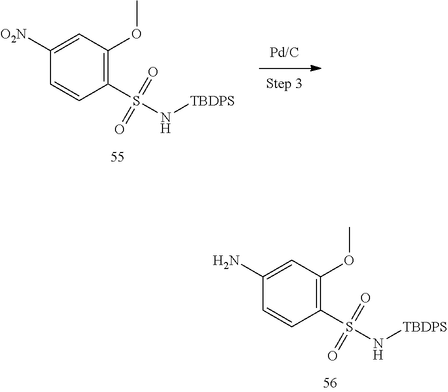

Into a 250 mL round-bottom flask were added N-(tert-butyldiphenylsilyl)-2-methoxy-4-nitrobenzenesulfonamide (12.6 g, 26.8 mmol, 1 equiv) and isopropanol (250 mL) at room temperature. Pd/C (580 mg, 5.5 mmol, 0.20 equiv) was added to the solution at room temperature under nitrogen atmosphere. The resulting mixture was stirred overnight at room temperature under hydrogen atmosphere, and then filtered. The filtrate was concentrated under reduced pressure to afforded 4-amino-N-(tert-butyldiphenylsilyl)-2-methoxybenzenesulfonamide (11.8 g, 84.6%) as yellow solid.

LCMS of 4-amino-N-(tert-butyldiphenylsilyl)-2-methoxybenzenesulfonamide (Method E): 441.2 [M+H]⁺, retention time 1.352 min.

### 4. Synthesis of 4-(N-(tert-butyldiphenylsilyl)sulfamoyl)-3-methoxybenzene-1-sulfonyl chloride

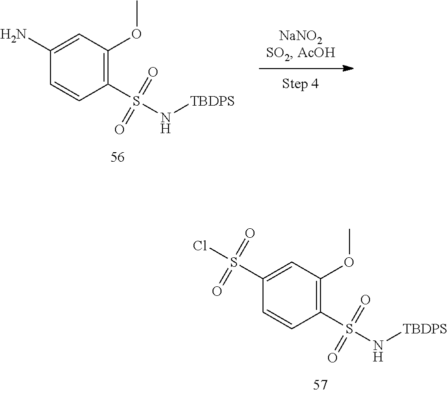

Into a 50 mL 3-necked round-bottom flask were added 4-amino-N-(tert-butyldiphenylsilyl)-2-methoxybenzenesulfonamide (2.0 g, 4.61 mmol, 1 equiv) in HCl (6 M, 20 mL) at room temperature. To this stirred solution was added NaNO₂ (382.8 mg, 5.55 mmol, 1.20 equiv) in portions at −10 degrees C. over 20 min. Then the resulting mixture was added to the solution of CuCl₂ in SO₂/AcOH (15 mL) (that had been stirred together for 15 min) in one portion at −10 degrees C. for 30 min. The resulting mixture was diluted with water (50 mL). The resulting mixture was extracted with CH₂ Cl₂ (3×25 mL). The combined organic layers were washed with water (3×50 mL), dried over anhydrous Na₂SO₄, and filtered. The filtrate was concentrated under reduced pressure. The crude product (2.8 g) was used in the next step directly without further purification.

### 5. Synthesis of N4-(3-(benzyloxy)propyl)-N1-(tert-butyldiphenylsilyl)-2-methoxybenzene-1,4-disulfonamide

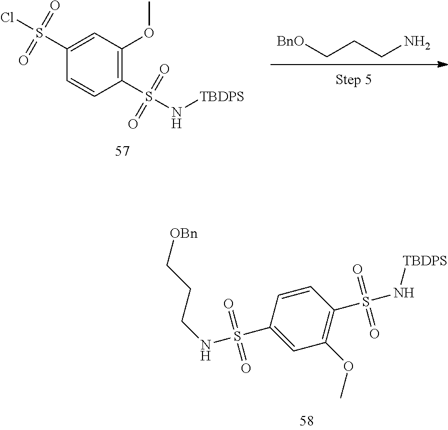

Into a 100 mL round-bottom flask were added 3-(benzyloxy)propan-1-amine in THE (40 mL, 0.5M) at 0 degrees C. To a stirred solution was added crude 2-(hydroxymethyl)-4-(methylsulfamoyl)benzene-1-sulfonyl chloride (2.8 g) in THE (10 mL) dropwise at 0 degrees C. The resulting mixture was stirred overnight at room temperature. The residue was applied onto a silica gel column which was eluted with ethyl acetate/petroleum ether (9:1) to give N4-(3-(benzyloxy)propyl)-N1-(tert-butyldiphenylsilyl)-2-methoxybenzene-1,4-disulfonamide (1.3 g, 44.0% for 2 steps) as yellow solid.

### 6. Synthesis of N1-(3-(benzyloxy)propyl)-3-methoxybenzene-1,4-disulfonamide

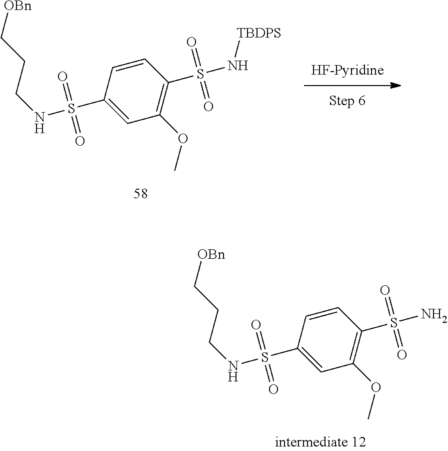

Into a 50-mL round-bottom flask, was placed a solution of N4-(3-(benzyloxy)propyl)-N1-(tert-butyldiphenylsilyl)-2-methoxybenzene-1,4-disulfonamide (274 mg, 0.42 mmol, 1 equiv) in THE (5 mL), and HF-Pyridine (417.9 mg, 4.22 mmol, 10 equiv). The resulting solution was stirred for 1 hr at room temperature. The resulting mixture was concentrated. The residue was applied onto a silica gel column which was eluted with MeOH/DCM (1:10) to give N1-(3-(benzyloxy)propyl)-3-methoxybenzene-1,4-disulfonamide (147.8 mg, 85.0%) as yellow solid.

LCMS of N1-(3-(benzyloxy)propyl)-3-methoxybenzene-1,4-disulfonamide (Method G): 413.1 [M−H]⁻, retention time 0.956 min.

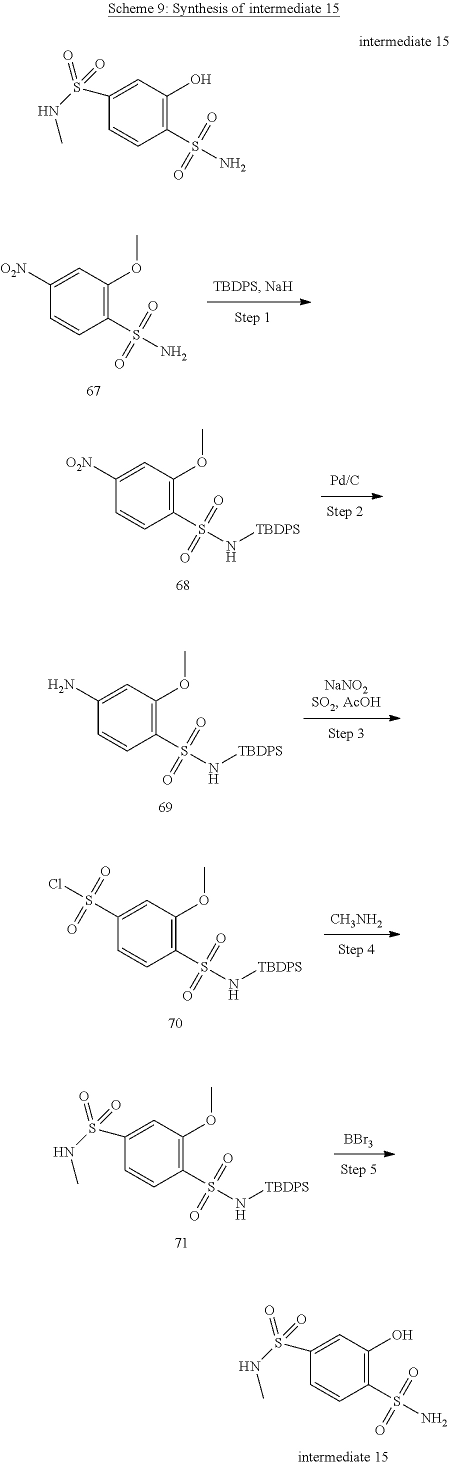

### 1. Synthesis of N-(tert-butyldiphenylsilyl)-2-methoxy-4-nitrobenzenesulfonamide

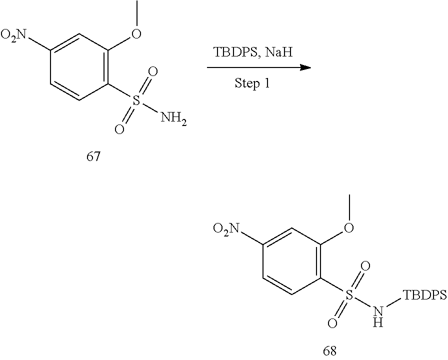

Into a 1 L round-bottom flask, was placed a solution of 2-methoxy-4-nitrobenzenesulfonamide (23.2 g, 100 mmol, 1 equiv) in THE (250 mL). NaH (8.0 g, 200.0 mmol, 2 equiv, 60%) was added to the solution in portions at 0° C. TBDPSCl (54.8 g, 200.0 mmol, 2 equiv) was added to the mixture at 0° C. The resulting solution was stirred overnight at room temperature. The reaction was then quenched by the addition of 1 L of ice-water. The resulting solution was extracted with 3×500 ml of ethyl acetate; the combined organic layers were dried over NaSO₄ and concentrated under vacuum. The residue was applied onto a silica gel column which was eluted with ethyl acetate/petroleum ether (1:5) to give N-(tert-butyldiphenylsilyl)-2-methoxy-4-nitrobenzenesulfonamide (28 g, 59.6%) as a yellow solid.

¹H NMR (300 MHz, CDCl3-d) δ 7.80 (d, J=2.1 Hz, 1H), 7.60-7.53 (m, 4H), 7.51 (dd, J=8.6, 2.0 Hz, 1H), 7.46-7.38 (m, 1H), 7.32-7.19 (m, 6H), 5.41 (s, 1H), 4.16 (s, 3H), 1.08 (s, 9H).

### 2. Synthesis of 4-amino-N-(tert-butyldiphenylsilyl)-2-methoxybenzenesulfonamide

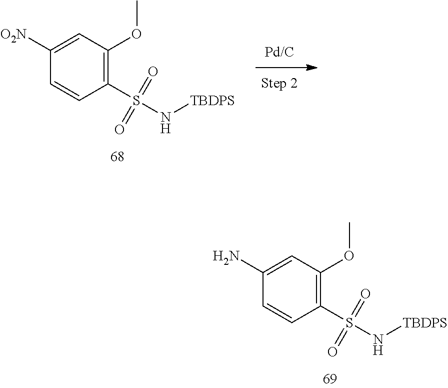

Into a 250 mL round-bottom flask were added N-(tert-butyldiphenylsilyl)-2-methoxy-4-nitrobenzenesulfonamide (12.6 g, 26.8 mmol, 1 equiv) and isopropanol(250 mL) at room temperature. Pd/C (580 mg, 5.5 mmol, 0.20 equiv) was added to the solution at room temperature under nitrogen atmosphere. The resulting mixture was stirred overnight at room temperature under hydrogen atmosphere, after which it was filtered. The filtrate was concentrated under reduced pressure to afforded 4-amino-N-(tert-butyldiphenylsilyl)-2-methoxybenzenesulfonamide (11.8 g, 84.6%) as yellow solid.

LCMS of 4-amino-N-(tert-butyldiphenylsilyl)-2-methoxybenzenesulfonamide (Method E): 441.2 [M+H]⁺, retention time 1.352 min.

### 3. Synthesis of 4-(N-(tert-butyldiphenylsilyl)sulfamoyl)-3-methoxybenzene-1-sulfonyl chloride

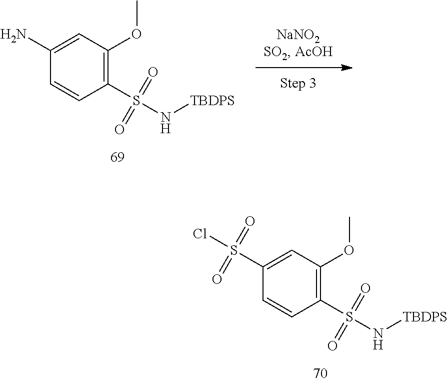

Into a 50 mL 3-necked round-bottom flask were added 4-amino-N-(tert-butyldiphenylsilyl)-2-methoxybenzenesulfonamide (2.0 g, 4.61 mmol, 1 equiv) in HCl (6 M, 20 mL) at room temperature. To this stirred solution was added NaNO₂ (382.8 mg, 5.55 mmol, 1.20 equiv) in portions at −10 degrees C. over 20 min. Then the resulting mixture was added to the solution of CuCl₂ in S02/AcOH(15 mL) (that had been stirred together for 15 min) in one portion at −10 degrees C. for 30 min. The resulting mixture was diluted with water (50 mL). The resulting mixture was extracted with CH₂ Cl₂ (3×25 mL). The combined organic layers were washed with water (3×50 mL), dried over anhydrous Na₂SO₄, and filtered. The filtrate was concentrated under reduced pressure. The crude product (2.8 g) was used in the next step directly without further purification.

### 4. Synthesis of N1-(tert-butyldiphenylsilyl)-2-methoxy-N4-methylbenzene-1,4-disulfonamide

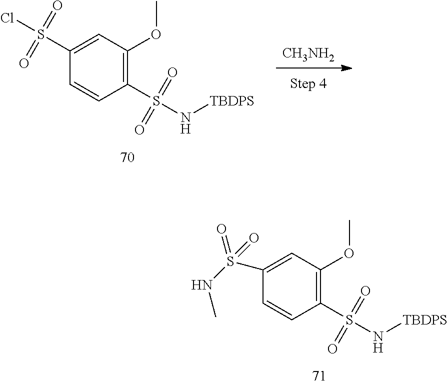

Into a 100 mL round-bottom flask was added methanamine in THE (40 mL, 0.5M) at 0 degrees C. To a stirred solution of NH3 in THF(40 mL) was added crude 4-(N-(tert-butyldiphenylsilyl)sulfamoyl)-3-methoxybenzene-1-sulfonyl chloride (2.8 g) in THF (10 mL) dropwise at 0 degrees C. The resulting mixture was stirred overnight at room temperature. The residue was applied onto a silica gel column which was eluted with ethyl acetate/petroleum ether (9:1) to give N1-(tert-butyldiphenylsilyl)-2-methoxy-N4-methylbenzene-1,4-disulfonamide (1.2 g, 52.0%) as yellow solid.

LCMS of N1-(tert-butyldiphenylsilyl)-2-methoxy-N4-methylbenzene-1,4-disulfonamide (Method F): 517.1 [M+H]⁻, retention time 1.454 min.

### 5. Synthesis of 3-(hydroxymethyl)-N1-methylbenzene-1,4-disulfonamide

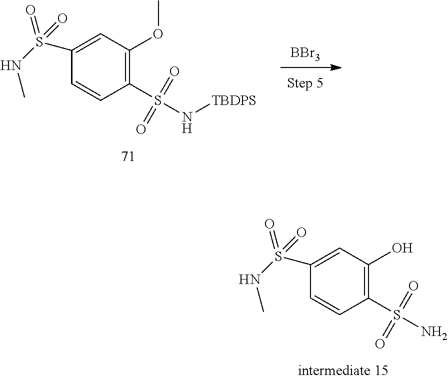

Into a 100 mL round-bottom flask was added N1-(tert-butyldiphenylsilyl)-2-methoxy-N4-methylbenzene-1,4-disulfonamide (518 mg, 1.0 mmol, 1 equiv) in DCM (20 mL). BBr₃ (1 M in DCM) was added to the solution in dropwise at 0° C., and the resulting mixture was stirred overnight at room temperature, after which it was purified by SiO₂-gel column chromatography [eluted with PE/EtOAc (1:1)] to afford 3-hydroxy-N1-methylbenzene-1,4-disulfonamide (160 mg, 60.1%) as a yellow solid. LCMS of N1-(tert-butyldiphenylsilyl)-2-methoxy-N4-methylbenzene-1,4-disulfonamide (Method F): 266.1 [M+H]⁻, retention time 0.454 min.

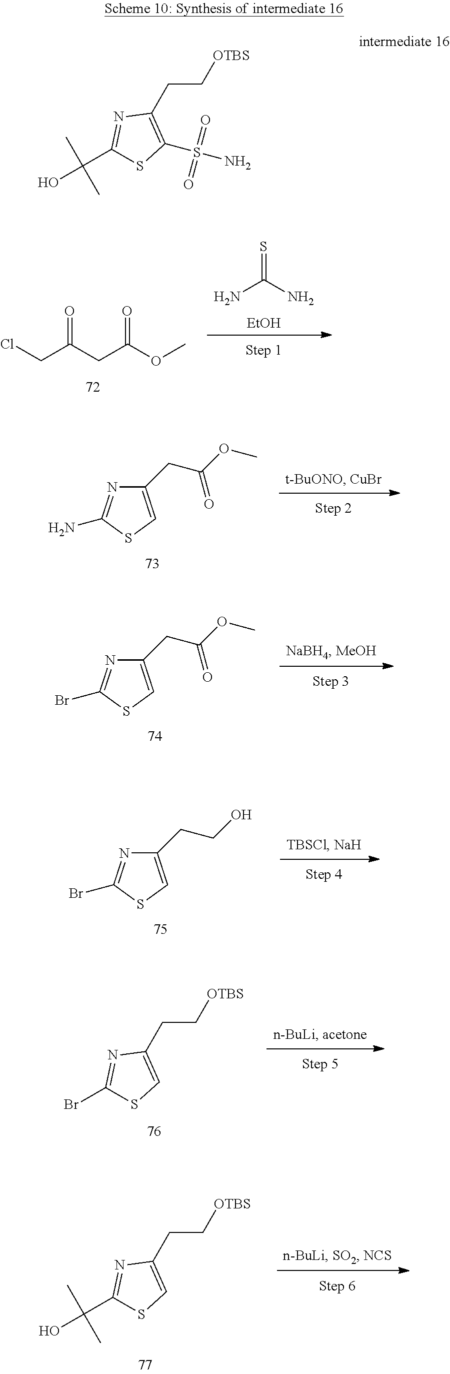

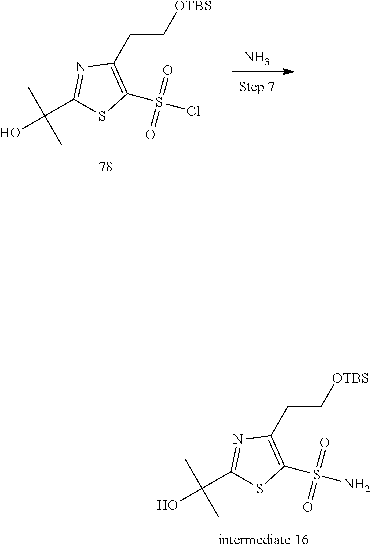

### 1. Synthesis of methyl 2-(2-aminothiazol-4-yl)acetate

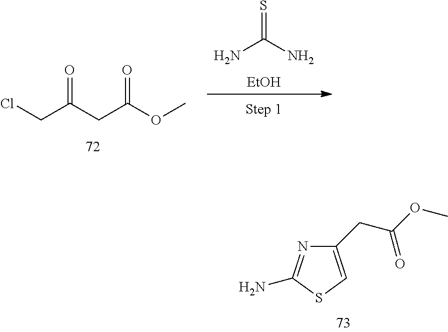

Into a 1 L round-bottom flask, was placed a solution of methyl 4-chloro-3-oxobutanoate (15.0 g, 100 mmol, 1 equiv) in EtOH (350 mL). Thiourea (7.6 g, 100 mmol, 1.0 equiv) was added to the solution. The resulting solution was refluxed overnight under stirring. The resulting mixture was cooled to room temperature and was filtered. The solid thus collected was washed with Et₂O (200 mL*2) and dried over oven at 50 degree overnight to give methyl 2-(2-aminothiazol-4-yl)acetate (15.4 g, 89.5%) as yellow solid.

H-NMR—methyl 2-(2-aminothiazol-4-yl)acetate: (CDCl3, 400 MHz, ppm): δ 6.35 (s, 1H), 5.25 (brs, 2H), 3.74 (s, 3H), 3.59 (s, 2H).

### 2. Synthesis of methyl 2-(2-bromothiazol-4-yl)acetate

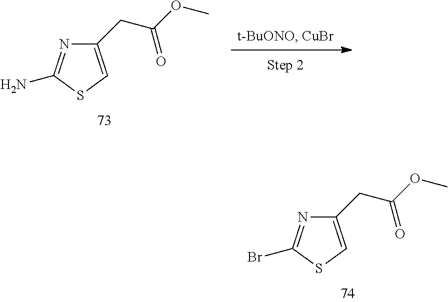

Into a 500 mL round-bottom flask, was placed a solution of methyl 2-(2-aminothiazol-4-yl)acetate (15.4 g, 89.5 mmol, 1 equiv) in MeCN(250 mL). CuBr was added to the solution, and then t-BuONO (3 eq.) was added to the solution dropwise at 0° C. The resulting solution was stirred for 30 min at room temperature and was then stirred for 2 h at 70° C. The resulting mixture was concentrated in vacuo and purified with silica gel column [eluted with EtOAc/PE (1:10)] to give methyl 2-(2-bromothiazol-4-yl)acetate (12.3 g, 58.2%) as white solid.

LCMS of methyl 2-(2-bromothiazol-4-yl)acetate (Method E): 236.0, 238.0 [M+H]⁻, retention time 0.924 min.

### 3. Synthesis of 2-(2-bromothiazol-4-yl)ethanol

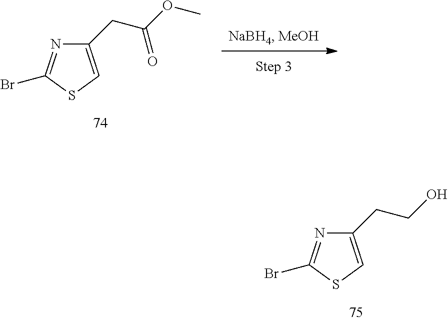

Into a 1 L round-bottom flask, was placed a solution of methyl 2-(2-bromothiazol-4-yl)acetate (12.3 g, 51.9 mmol, 1 equiv) in EtOH (200 mL). NaBH₄ (3.9 g, 103.8 mmol, 2 equiv) was added to the solution in portions at 0° C. The resulting solution was stirred for 3 hr at room temperature. The reaction was then quenched by the addition of 1 L of ice-water. The resulting solution was extracted with 3×500 ml of ethyl acetate. The combined organic layers were dried over NaSO₄ and concentrated under vacuum. This resulted in 8.9 g (82.1%) of 2-(2-bromothiazol-4-yl)ethanol as yellow oil.

LCMS of 2-(2-bromothiazol-4-yl)ethanol (Method G): 208.0, 210.0 [M+H]⁻, retention time 0.771 min.

### 4. Synthesis of 2-bromo-4-(2-(tert-butyldimethylsilyloxy)ethyl)thiazole

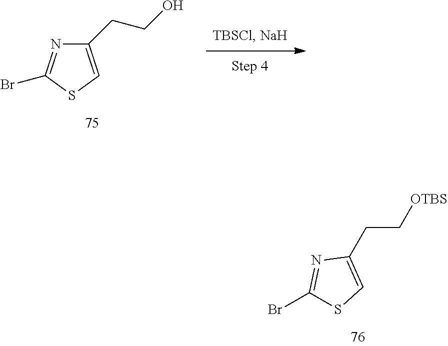 [SMILES: OCCc1csc(Br)n1 ; CC(C)(C)[Si](C)(C)OCCc1csc(Br)n1]

Into a 500 mL round-bottom flask, was placed a solution of 2-(2-bromothiazol-4-yl)ethanol (8.9 g, 42.6 mmol, 1 equiv) in THE (400 mL). NaH (2.56 g, 63.9 mmol, 1.5 equiv, 60%) was added to the mixture in portions at 0° C. The mixture was stirred at 0° C. for another 1 h, after which TBSCl (10.2 g, 68.2 mmol, 1.6 equiv) was added in portions at 0° C. The resulting solution was stirred for 2 hr at room temperature. The reaction was then quenched by the addition of 300 mL of ice-water. The resulting solution was extracted with 3×300 ml of ethyl acetate; the combined organic phase was dried over NaSO₄ and concentrated. The residue was applied onto a silica gel column with ethyl acetate/petroleum ether (1:30). This resulted in 7.6 g (55.1%) of 2-bromo-4-(2-(tert-butyldimethylsilyloxy)ethyl)thiazole as yellow oil.

### 5. Synthesis of 2-(4-(2-(tert-butyldimethylsilyloxy)ethyl)thiazol-2-yl)propan-2-ol

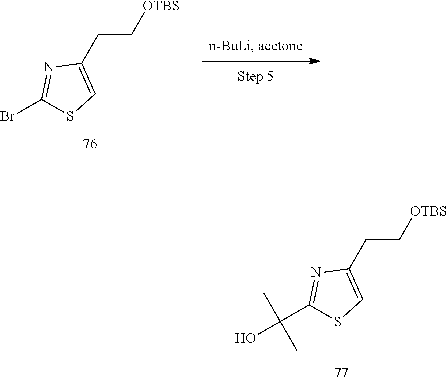 [SMILES: CC(C)(C)[Si](C)(C)OCCc1csc(Br)n1 ; CC(C)(O)c1nc(CCO[Si](C)(C)C(C)(C)C)cs1]

Into a 500-mL round-bottom flask purged with and maintained under an inert atmosphere of nitrogen, was placed a solution of 2-bromo-4-(2-(tert-butyldimethylsilyloxy)ethyl)thiazole (7.6 g, 23.4 mmol, 1 equiv) in THE (50 mL). n-BuLi (11.2 mL, 28.1 mmol, 2.5 M, 1.2 equiv) was added to the mixture in dropwise at −78° C.; and the resulting solution was stirred for 30 min at −78° C. Then acetone (1.6 g, 28.1 mmol, 1.2 equiv) was added dropwise at −78° C. and stirred for another 1 h at room temperature. The reaction was then quenched by the addition of 200 mL of water. The resulting solution was extracted with 3×300 ml of ethyl acetate; the combined organic phase was dried over NaSO₄ and concentrated. The residue was applied onto a silica gel column with ethyl acetate/petroleum ether (1:10). This resulted in 6.1 g (86.2%) of 2-(4-(2-(tert-butyldimethylsilyloxy)ethyl)thiazol-2-yl)propan-2-ol as yellow oil.

LCMS of 2-(4-(2-(tert-butyldimethylsilyloxy)ethyl)thiazol-2-yl)propan-2-ol (Method G): 302.1 [M+H]⁻, retention time 1.364 min.

### 6. Synthesis of 4-(2-(tert-butyldimethylsilyloxy)ethyl)-2-(2-hydroxypropan-2-yl)thiazole-5-sulfonyl chloride

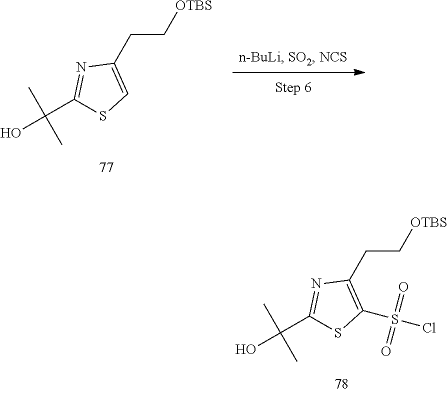 [SMILES: CC(C)(O)c1nc(CCO[Si](C)(C)C(C)(C)C)cs1 ; CC(C)(O)c1nc(CCO[Si](C)(C)C(C)(C)C)c(S(=O)(=O)Cl)s1]

Into a 250-mL round-bottom flask purged with and maintained under an inert atmosphere of nitrogen, was placed a solution of 2-(4-(2-(tert-butyldimethylsilyloxy)ethyl)thiazol-2-yl)propan-2-ol (6.1 g, 20.2 mmol, 1 equiv) in THE (100 mL). n-BuLi (24.2 mL, 60.6 mmol, 2.5 M, 3 equiv) was added to the mixture at −78° C. and the resulting mixture stirred for another 30 min at −78° C. Then SO₂ was bubbled for 30 min and stirred for another 2 h at room temperature. The resulting mixture was concentrated. The residue thus obtained was dissolved in DCM (200 mL), whereto NCS (5.39 g, 40.4 mmol, 2 equiv) was added in portions at 0° C. The resulting mixture was stirred for another 30 min at 0° C. and was concentrated at 0° C. This resulted in 4-(2-(tert-butyldimethylsilyloxy)ethyl)-2-(2-hydroxypropan-2-yl)thiazole-5-sulfonyl chloride (12.5 g) as a yellow solid which was used directly for the next step.

### 7. Synthesis of 4-(2-(tert-butyldimethylsilyloxy)ethyl)-2-(2-hydroxypropan-2-yl)thiazole-5-sulfonamide

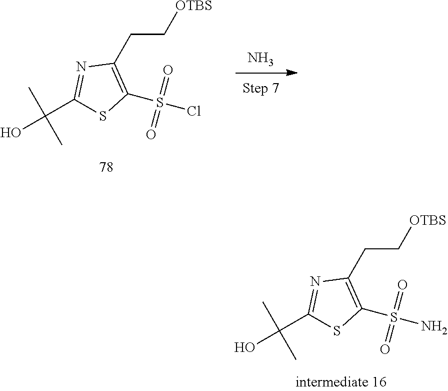

Into a 250-mL round-bottom flask, was placed a solution of 4-(2-(tert-butyldimethylsilyloxy)ethyl)-2-(2-hydroxypropan-2-yl)thiazole-5-sulfonyl chloride (12.5 g, 32.38 mmol, 1 equiv) in DCM (130 mL). NH3 was bubbled for 10 min. The resulting solution was stirred for another 1 hr at room temperature, after which it was concentrated. The resulting residue was applied onto a silica gel column which was eluted with ethyl acetate/petroleum ether (1:5). This resulted in 4.8 g (62.5% for 2 steps) of 4-(2-(tert-butyldimethylsilyloxy)ethyl)-2-(2-hydroxypropan-2-yl)thiazole-5-sulfonamide as yellow oil.

LCMS of 4-(2-(tert-butyldimethylsilyloxy)ethyl)-2-(2-hydroxypropan-2-yl)thiazole-5-sulfonamide (Method G): 383.1 [M+H]⁻, retention time 1.747 min.

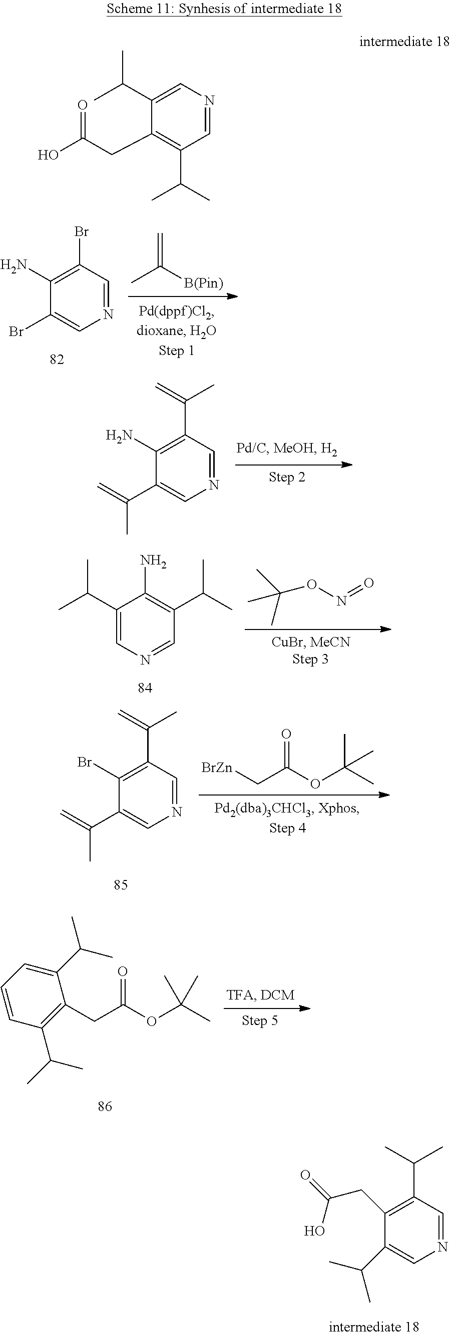

### 1. Synthesis of 3,5-bis(prop-1-en-2-yl)pyridin-4-amine

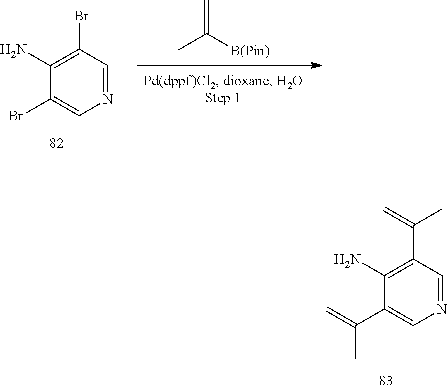

Into a 500-mL round-bottom flask, was placed 3,5-dibromopyridin-4-amine (5 g, 19.85 mmol, 1.00 equiv), dioxane (150 mL), water(15 mL), 4,4,5,5-tetramethyl-2-(prop-1-en-2-yl)-1,3,2-dioxaborolane (10.08 g, 59.99 mmol, 3.00 equiv), Cs₂CO₃ (19.56 g, 60.03 mmol, 3.00 equiv), and Pd(dppf)Cl₂ (1.46 g, 2.00 mmol) under an atmosphere of nitrogen. The resulting solution was stirred for 15 h at 90° C. in an oil bath, after which it was concentrated under vacuum. The residue thus obtained was applied onto a silica gel column with ethyl acetate/petroleum ether (1:3). This resulted in 3.0 g (87%) of 3,5-bis(prop-1-en-2-yl)pyridin-4-amine as light yellow oil.

LCMS of 3,5-bis(prop-1-en-2-yl)pyridin-4-amine (Method A): 175.1 [M+H]⁺, retention time 0.872 min.

### 2. Synthesis of 3,5-bis(propan-2-yl)pyridin-4-amine

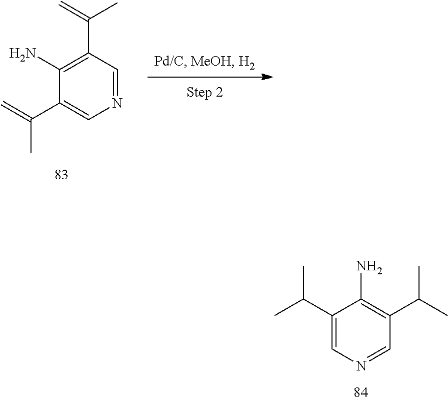

Into a 250-mL round-bottom flask, was placed 3,5-bis(prop-1-en-2-yl)pyridin-4-amine (3.0 g, 17.22 mmol, 1.00 equiv), methanol (50 mL), and palladium on carbon (300 mg) under a hydrogen atmosphere. The reaction mixture was stirred overnight at room temperature, after which it was filtered. The filtrate was concentrated under vacuum. This resulted in 2.8 g (91%) of 3,5-bis(propan-2-yl)pyridin-4-amine as a light yellow solid.

LCMS of 3,5-bis(propan-2-yl)pyridin-4-amine (Method A): 179.1 [M+H]⁺, retention time 0.912 min.

### 3. Synthesis of 4-bromo-3,5-bis(propan-2-yl)pyridine

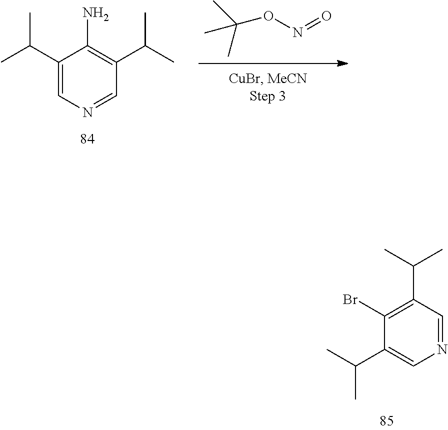

Into a 100-mL 3-necked round-bottom flask, was placed CuBr (1.7 g, 3.00 equiv) and ACN (45 mL). This was followed by the addition of tert-butyl nitrite (824 mg, 7.99 mmol, 2.00 equiv) dropwise with stirring at 0° C. The resulting solution was stirred for 10 min at 70° C. in an oil bath. To this was then added a solution of 3,5-bis(propan-2-yl)pyridin-4-amine (712 mg, 3.99 mmol, 1.00 equiv) in ACN (5 mL) dropwise with stirring at 70° C. The resulting solution was allowed to react, with stirring, for an additional 30 min while the temperature was maintained at 70° C. in an oil bath. The resulting mixture was concentrated under vacuum. The residue thus obtained was treated with ethyl acetate (100 mL) and sodium bicarbonate(aq) (30 mL). The resulting mixture was allowed to react, with stirring, for an additional 30 min at room temperature. The solids were filtered out. The filtrate was extracted with 3×100 mL of ethyl acetate; and the combined organic layers were concentrated under vacuum. The residue thus obtained was applied onto a silica gel column with ethyl acetate/petroleum ether (1:4). This resulted in 450 mg (47%) of 4-bromo-3,5-bis(propan-2-yl)pyridine as yellow oil.

LCMS of 4-bromo-3,5-bis(propan-2-yl)pyridine (Method A): 242.1, 244.1 [M+H]⁺, retention time 1.114 min.

### 4. Synthesis of tert-butyl 2-[3,5-bis(propan-2-yl)pyridin-4-yl]acetate

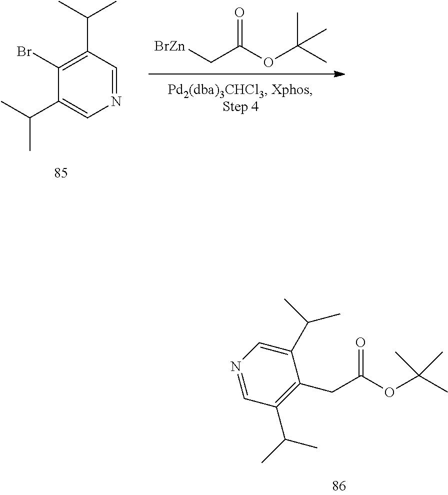

Into a 50-mL round-bottom flask, was placed 4-bromo-3,5-bis(propan-2-yl)pyridine (115 mg, 0.47 mmol, 1.00 equiv), Xphos (23 mg, 0.10 equiv), Pd₂(dba)₃CHCl₃ (25 mg, 0.05 equiv), tert-butyl 2-(bromozincio)acetate (248 mg, 0.95 mmol, 2.00 equiv), tetrahydrofuran (30 mL), N₂. The resulting solution was stirred for 2 h at 70° C. in an oil bath. The reaction was then quenched by the addition of 20 mL of NH₄Cl(aq). The resulting solution was extracted with 3×30 mL of ethyl acetate and the organic layers combined and concentrated under vacuum. The residue was applied onto a silica gel column with ethyl acetate/petroleum ether (2:3). This resulted in 120 mg (91%) of tert-butyl 2-[3,5-bis(propan-2-yl)pyridin-4-yl]acetate as yellow oil.

### 5. Synthesis of 2-[3,5-bis(propan-2-yl)pyridin-4-yl]acetic acid

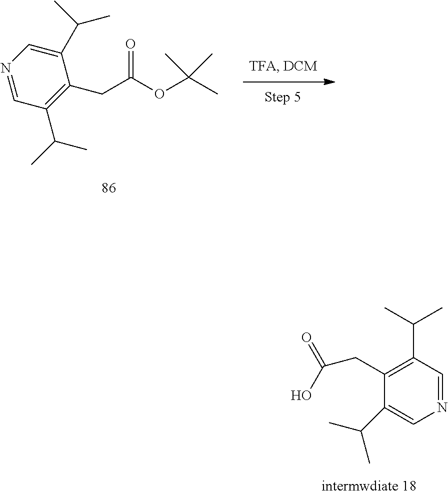 [SMILES: CC(C)c1cncc(C(C)C)c1CC(=O)OC(C)(C)C ; CC(C)c1cncc(C(C)C)c1CC(=O)O]

Into a 50-mL round-bottom flask, was placed tert-butyl 2-[3,5-bis(propan-2-yl)pyridin-4-yl]acetate (120 mg, 0.43 mmol, 1.00 equiv), dichloromethane (2 mL), and trifluoroacetic acid (2 mL). The resulting solution was stirred for 1 h at room temperature. The resulting mixture was concentrated under vacuum. This resulted in 100 mg of 2-[3,5-bis(propan-2-yl)pyridin-4-yl]acetic acid as yellow oil.

LCMS of 2-[3,5-bis(propan-2-yl)pyridin-4-yl]acetic acid (Method N): 222.2 [M+H]⁺, retention time 0.702 min.

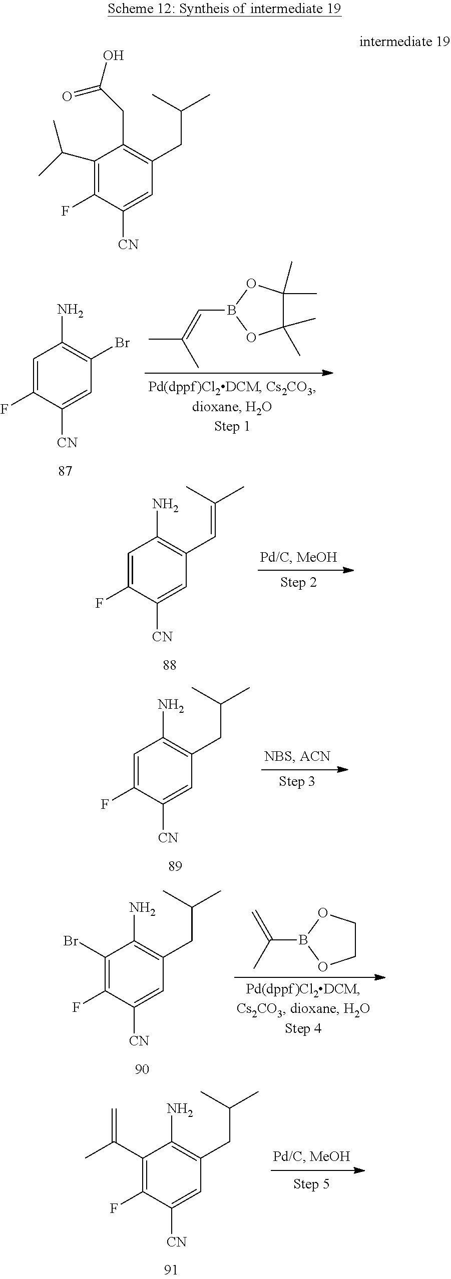

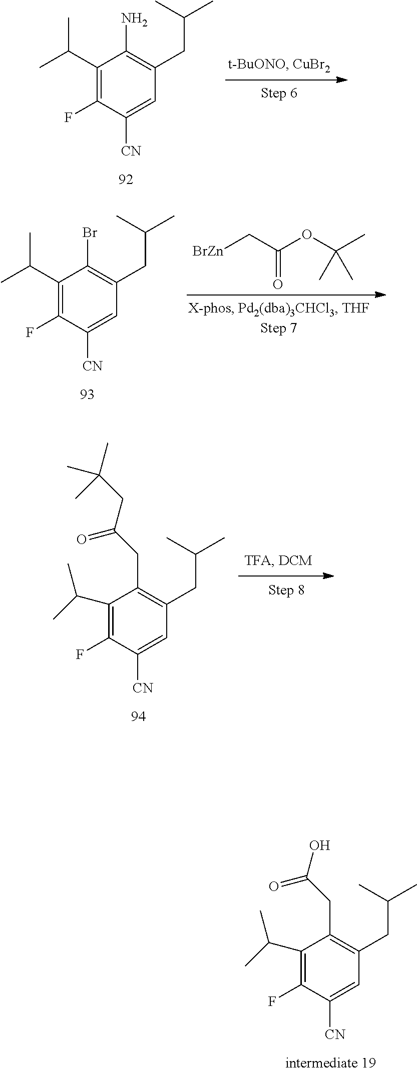

### 1. Synthesis of 4-amino-2-fluoro-5-(2-methylprop-1-en-1-yl)benzonitrile

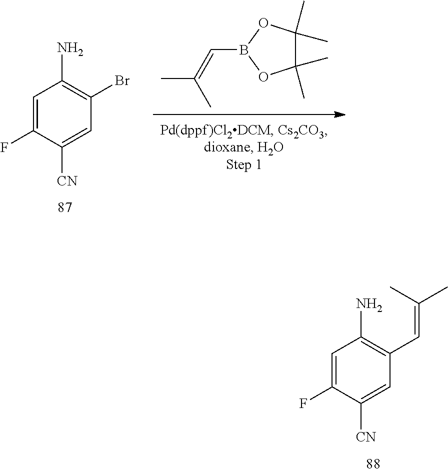

To a stirred solution/mixture of 4-amino-5-bromo-2-fluorobenzonitrile (1 g, 4.65 mol, 1 equiv), 4,4,5,5-tetramethyl-2-(2-methylprop-1-en-1-yl)-1,3,2-dioxaborolane (1.3 g, 6.98 mmol, 1.50 equiv), and Cs₂CO₃ (4.5 g, 13.95 mmol, 3.00 equiv) in dioxane (15 mL) and H₂O (0.6 mL) was added Pd(dppf)Cl₂-DCM (759.9 mg, 0.93 mmol, 0.2 equiv) at room temperature under nitrogen atmosphere. The resulting mixture was stirred overnight at 90° C. under nitrogen atmosphere. The resulting mixture was concentrated under vacuum. The residue was purified by silica gel column chromatography, eluted with PE/EtOAc (12:1) to afford 4-amino-2-fluoro-5-(2-methylprop-1-en-1-yl)benzonitrile(660 mg, 74.61%) as a yellow solid.

LC-MS-4-amino-2-fluoro-5-(2-methylprop-1-en-1-yl)benzonitrile (Method M): (ES, m/z): [M+H]⁺=191.1, retention time 1.056 min.

### 2. Synthesis of 4-amino-2-fluoro-5-(2-methylpropyl)benzonitrile

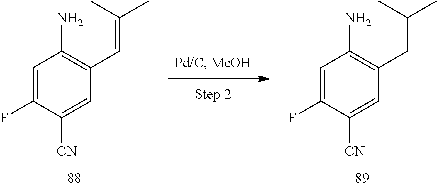

A solution/mixture of 4-amino-2-fluoro-5-(2-methylprop-1-en-1-yl)benzonitrile (1.98 g, 10410 mmol, 1 equiv) and Pd/C(553.9 mg, 5.20 mmol, 0.50 equiv) in MeOH (150 mL) was stirred for 2 days at room temperature under argon atmosphere. The resulting mixture was filtered, and the filter cake was washed with MeOH (3×20 mL). The filtrate was concentrated under reduced pressure. This resulted in 4-amino-2-fluoro-5-(2-methylpropyl)benzonitrile (1.9 g, crude) as a yellow solid.

LC-MS-4-amino-2-fluoro-5-(2-methylpropyl)benzonitrile (Method M): (ES, m/z): [M+H]⁺=193.1, retention time 1.462 min.

### 3. Synthesis of 4-amino-3-bromo-2-fluoro-5-(2-methylpropyl)benzonitrile

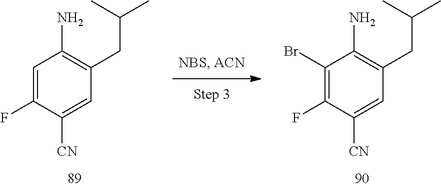

A solution/mixture of 4-amino-2-fluoro-5-(2-methylpropyl)benzonitrile (1.9 g, 9.88 mmol, 1 equiv) and NBS (2.6 g, 14.83 mmol, 1.50 equiv) in ACN (50 mL) was stirred for 3 h at 65° C. The resulting mixture was concentrated under vacuum. The residue was purified by silica gel column chromatography, eluted with PE/EtOAc (100:1) to afford 4-amino-3-bromo-2-fluoro-5-(2-methylpropyl)benzonitrile (2 g, 74.63%) as a yellow solid.

LC-MS-4-amino-3-bromo-2-fluoro-5-(2-methylpropyl)benzonitrile (Method G): (ES, m/z): [M+H]⁺=271.0, retention time 1.271 min.

### 4. Synthesis of 4-amino-2-fluoro-5-(2-methylpropyl)-3-(prop-1-en-2-yl)benzonitrile

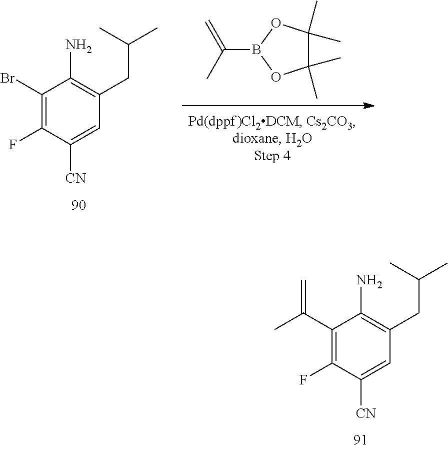 [SMILES: CC(C)Cc1cc(C#N)c(F)c(Br)c1N ; C=C(C)B1OC(C)(C)C(C)(C)O1 ; C=C(C)c1c(N)c(CC(C)C)cc(C#N)c1F]

To a stirred solution/mixture of 4-amino-3-bromo-2-fluoro-5-(2-methylpropyl)benzonitrile (2 g, 7.38 mol, 1 equiv), 4,4,5,5-tetramethyl-2-(prop-1-en-2-yl)-1,3,2-dioxaborolane (1.9 g, 11.06 mol, 1.50 equiv), and Cs₂CO₃ (7.2 g, 22.10 mmol, 3.00 equiv) in dioxane (13 mL) and H₂O (0.6 mL) was added Pd(dppf)Cl₂-DCM (1.2 g, 1.47 mol, 0.20 equiv) at room temperature under nitrogen atmosphere. The resulting mixture was stirred overnight at 90° C. under nitrogen atmosphere. The resulting mixture was concentrated under vacuum. The residue was purified by silica gel column chromatography, eluted with PE/EtOAc (50:1) to afford 4-amino-2-fluoro-5-(2-methylpropyl)-3-(prop-i-en-2-yl)benzonitrile(1.1 g, 64.19%) as a yellow solid.

LC-MS-4-amino-2-fluoro-5-(2-methylpropyl)-3-(prop-i-en-2-yl)benzonitrile (Method G): (ES, m/z): [M−H]⁻=231.2, retention time 1.317 min.

### 5. Synthesis of 4-amino-2-fluoro-5-(2-methylpropyl)-3-(propan-2-yl)benzonitrile

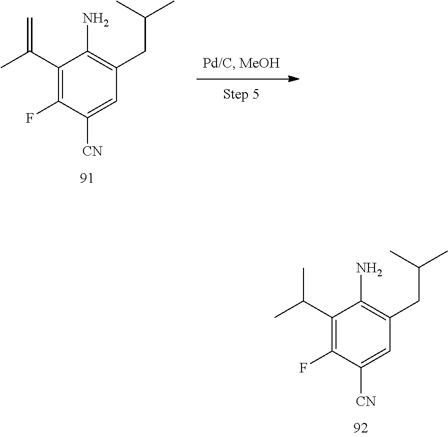 [SMILES: C=C(C)c1c(N)c(CC(C)C)cc(C#N)c1F ; CC(C)Cc1cc(C#N)c(F)c(C(C)C)c1N]

A solution/mixture of 4-amino-2-fluoro-5-(2-methylpropyl)-3-(prop-i-en-2-yl)benzonitrile (1.1 mg, 1 equiv) and Pd/C (503.9 mg, 4.74 mmol, 1.00 equiv) in MeOH (80 mL) was stirred for 2 days at room temperature under nitrogen atmosphere. The residue was purified by Prep-TLC (PE/EtOAc 2:1) to afford 4-amino-2-fluoro-5-(2-methylpropyl)-3-(propan-2-yl)benzonitrile (1.1 g, 99.14%) as a yellow solid.

LC-MS-4-amino-2-fluoro-5-(2-methylpropyl)-3-(propan-2-yl)benzonitrile (Method G): (ES, m/z): [M−H]⁻=233.1, retention time 1.962 min.

### 6. Synthesis of 4-bromo-2-fluoro-5-(2-methylpropyl)-3-(propan-2-yl)benzonitrile

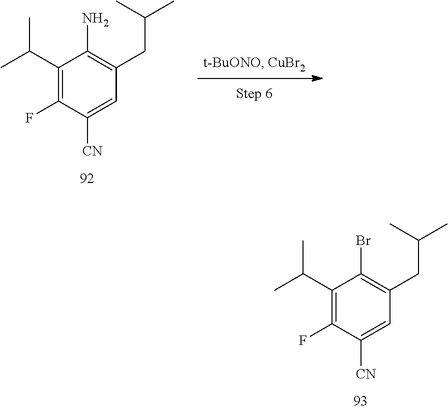 [SMILES: CC(C)Cc1cc(C#N)c(F)c(C(C)C)c1N ; CC(C)Cc1cc(C#N)c(F)c(C(C)C)c1Br]

To a stirred solution/mixture of 4-amino-2-fluoro-5-(2-methylpropyl)-3-(propan-2-yl)benzonitrile (1.1 g, 4.69 mol, 1 equiv) and CuBr (1.3 g, 9.39 mmol, 2.00 equiv) in ACN (30 mL) was added nitrous acid tert-butyl ester (1.0 g, 9.70 mmol, 2.07 equiv) at 0° C. under nitrogen atmosphere. The resulting mixture was stirred for 3 min at 0° C. under nitrogen atmosphere. The resulting mixture was stirred for 3 h at 60° C. under nitrogen atmosphere. The residue was purified by Prep-TLC (PE/EtOAc 10:1) to afford 4-bromo-2-fluoro-5-(2-methylpropyl)-3-(propan-2-yl)benzonitrile (700 mg, 50.0%) as a yellow solid.

¹H NMR-4-bromo-2-fluoro-5-(2-methylpropyl)-3-(propan-2-yl)benzonitrile (300 MHz, DMSO-d6) δ 7.77 (d, J=6.0 Hz, 1H), 3.69-3.60 (m, 1H), 2.67-2.65 (m, 2H), 1.98-1.89 (m, 1H), 1.33-1.30 (m, 6H), 0.91-0.89 (m, 6H).

### 7. Synthesis of 2-[4-cyano-3-fluoro-6-(2-methylpropyl)-2-(propan-2-yl)phenyl]acetate

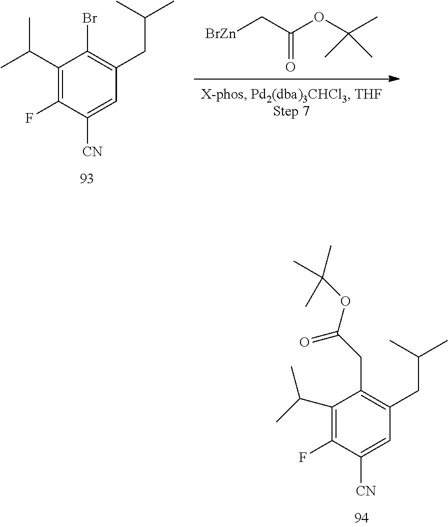

A solution/mixture of 4-bromo-2-fluoro-5-(2-methylpropyl)-3-(propan-2-yl)benzonitrile (700 mg, 2.35 mmol, 1 equiv), X-phos (111.9 mg, 0.23 mmol, 0.10 equiv) and Pd₂(dba)₃CHCl₃ (121.5 mg, 0.12 mmol, 0.05 equiv) in THE (15 mL) was stirred for 10 min at room temperature under nitrogen atmosphere. To the above mixture was added tert-butyl 2-(bromozincio)acetate (1222.7 mg, 4.69 mmol, 2.00 equiv). The resulting mixture was stirred for additional 3 h at 65° C., after which it was purified by silica gel column chromatography, eluted with PE/EtOAc (50:1) to afford tert-butyl 2-[4-cyano-3-fluoro-6-(2-methylpropyl)-2-(propan-2-yl)phenyl]acetate (500 mg, 63.88%) as a yellow oil.

### 8. Synthesis of 2-[4-cyano-3-fluoro-6-(2-methylpropyl)-2-(propan-2-yl)phenyl]acetic acid

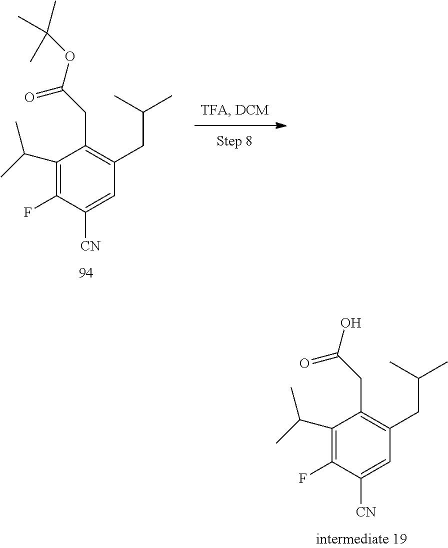 [SMILES: CC(C)Cc1cc(C#N)c(F)c(C(C)C)c1CC(=O)OC(C)(C)C ; CC(C)Cc1cc(C#N)c(F)c(C(C)C)c1CC(=O)O]

A solution/mixture of tert-butyl 2-[4-cyano-3-fluoro-6-(2-methylpropyl)-2-(propan-2-yl)phenyl]acetate (500 mg, 1.50 mmol, 1 equiv) and TFA (10 mL) in DCM (10 mL) was stirred for 3 h at room temperature. The resulting mixture was concentrated under vacuum. The residue was purified by Prep-TLC (CH₂Cl₂/MeOH 10:1) to afford 2-[4-cyano-3-fluoro-6-(2-methylpropyl)-2-(propan-2-yl)phenyl]acetic acid (500 mg, crude) as a yellow solid.

LC-MS-2-[4-cyano-3-fluoro-6-(2-methylpropyl)-2-(propan-2-yl)phenyl]acetic acid: (ES, m/z): [M−H]⁻=276.2

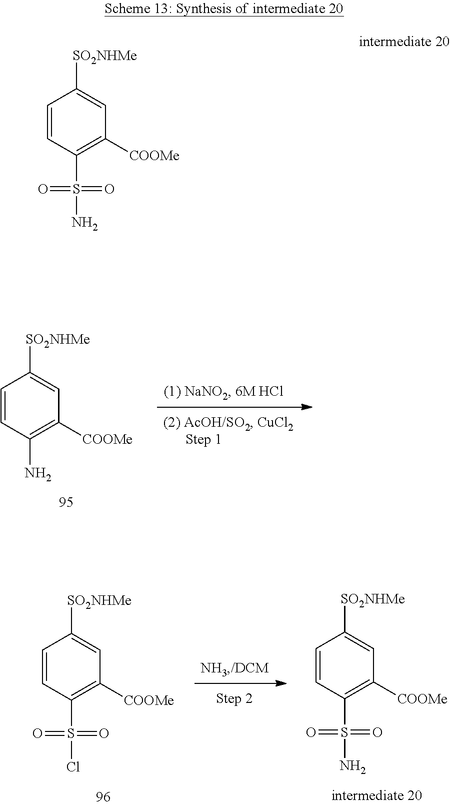

### 1. Synthesis of methyl 2-(chlorosulfonyl)-5-(methylsulfamoyl)benzoate

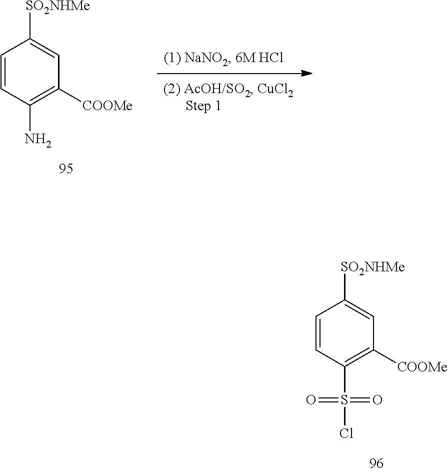

Into a 250-mL round-bottom flask, was placed methyl 2-amino-5-(methylsulfamoyl)benzoate (2 g), HCl(20 mL, aqueous, 6 M), NaNO₂ (1.2 g), SO₂/CH₃COOH (20 mL), and CuCl₂ (550 mg). The resulting solution was stirred for 2 hours at 0 degrees C. The residue was applied onto a silica gel column with ethyl acetate/petroleum ether (1/1). This resulted in 600 mg of methyl 2-(chlorosulfonyl)-5-(methylsulfamoyl)benzoate as a solid.

### 2. Synthesis of methyl 5-(methylsulfamoyl)-2-sulfamoylbenzoate

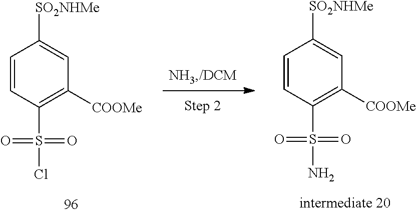

Into a 250-mL round-bottom flask, was placed methyl 2-(chlorosulfonyl)-5-(methylsulfamoyl)benzoate (300 mg), NH3/THF (20 mL). The resulting solution was stirred for 4 hours at room temperature. The residue was applied onto a silica gel column with ethyl acetate/petroleum ether (1/1). This resulted in 300 mg of methyl 5-(methylsulfamoyl)-2-sulfamoylbenzoate as a white solid.

LC-MS—methyl 5-(methylsulfamoyl)-2-sulfamoylbenzoate (Method M): 307 [M−H]⁻ retention time 0.656 min.

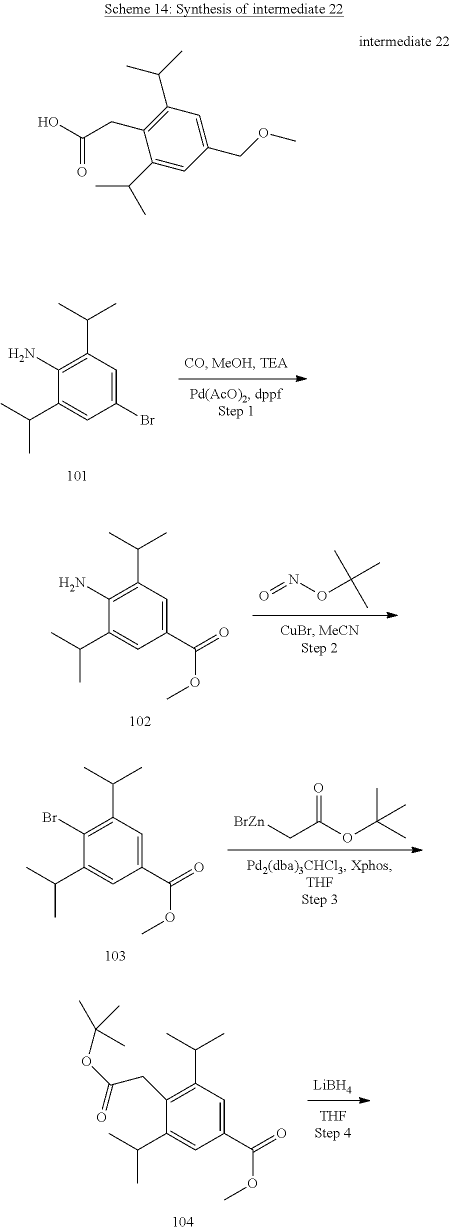

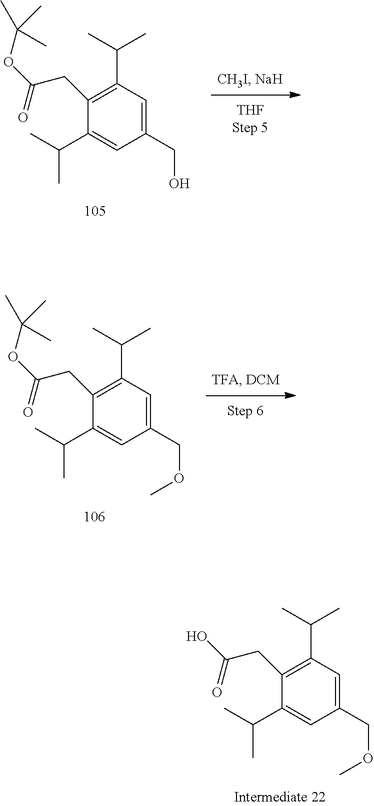

### 1. Synthesis of methyl 4-amino-3,5-diisopropylbenzoate

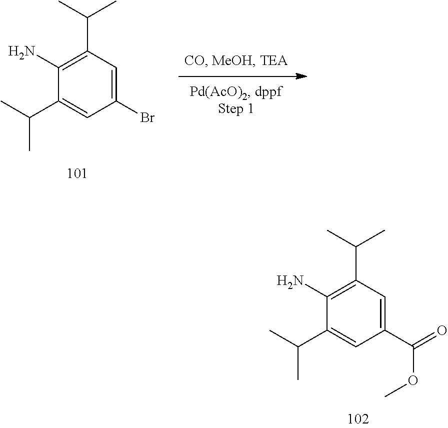 [SMILES: CC(C)c1cc(Br)cc(C(C)C)c1N ; COC(=O)c1cc(C(C)C)c(N)c(C(C)C)c1]

Into a 1-L autoclave was placed a solution of 4-bromo-2,6-diisopropylbenzenamine (10 g, 39 mmol) in MeOH (300 mL). To the solution were added Pd(OAc)₂ (1.75 g, 7.8 mmol), dppf (4.3 g, 7.8 mmol), and TEA (20 g, 195 mmol). After sealing the autoclave, the gas was exchanged with CO for 3 times. The reaction was stirred at 120° C. overnight. After cooling the reaction mixture, the gas was exchanged with N2, the reaction was concentrated and diluted with water (300 mL). The resulting solution was extracted with EtOAc (3×200 mL). The combined organic layers were dried over anhydrous Na₂SO₄ and concentrated. The residue thus obtained was purified on a SiO₂-gel column and eluted with ethyl acetate/petroleum ether (1:10 to 1:5). This resulted in 5.6 g (62%) of the title compound as a brown oil.

LCMS of methyl 4-amino-3,5-diisopropylbenzoate (Method B): 236.2 [M+H]⁺, retention time 0.805 min.

### 2. Synthesis of methyl 4-bromo-3,5-diisopropylbenzoate

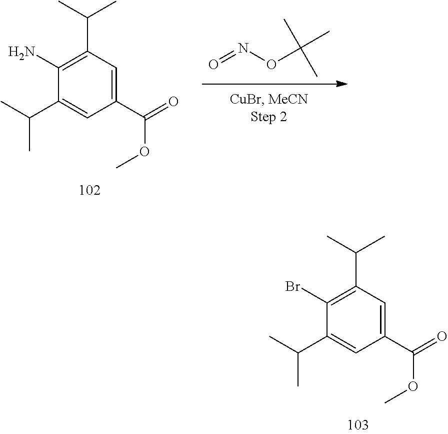

Into a 250 mL round-bottom flask, was placed a solution of methyl 4-amino-3,5-diisopropylbenzoate (5.6 g, 23.8 mmol, 1 equiv) in MeCN (100 mL). CuBr₂ (10.6 g, 47.6 mmol, 2 equiv) was added to the solution, followed by t-BuONO (4.8 g, 47.6 mmol, 2 equiv) which was added dropwise at 0° C. The resulting solution was stirred for 30 min at room temperature and then stirred for 2 h at 70° C. The resulting mixture was concentrated in vacuo and purified with silica gel column chromatography [eluted with EtOAc/PE (1:10)] to give methyl 4-bromo-3,5-diisopropylbenzoate (3.4 g, 47.9%) as white solid.

¹H NMR (400 MHz, DMSO-d6) δ 7.70 (s, 2H), 3.91 (s, 2H), 3.83 (s, 3H), 3.47-3.35 (m, 2H), 1.19 (d, J=6.0 Hz, 12H).

### 3. Synthesis of methyl 4-(2-(tert-butoxy)-2-oxoethyl)-3,5-diisopropylbenzoate

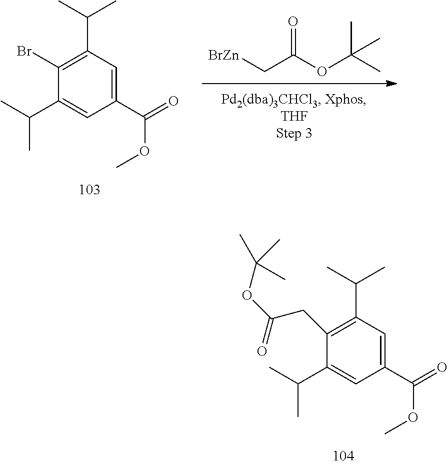

Into a 100 mL round-bottom flask was placed a solution of methyl 4-bromo-3,5-diisopropylbenzoate (3.4 g, 11.4 mmol, 1 equiv) in THE (100 mL). Pd₂(dba)₃ (1.0 g, 1.1 mmol, 0.1 equiv) and (2-tert-butoxy-2-oxoethyl)zinc(II) bromide (4.4 g, 17.1 mmol, 1.5 equiv) were added to the solution under N2 atmosphere. The resulting solution was stirred for 2 h at 70° C., after which it was concentrated in vacuo and purified with silica gel column chromatography [eluted with EtOAc/PE (1:10)] to give methyl 4-(2-(tert-butoxy)-2-oxoethyl)-3,5-diisopropylbenzoate (2.0 g, 44.4%) as white solid.

### 4. Synthesis of Tert-butyl 2-(4-(hydroxymethyl)-2,6-diisopropylphenyl)acetate

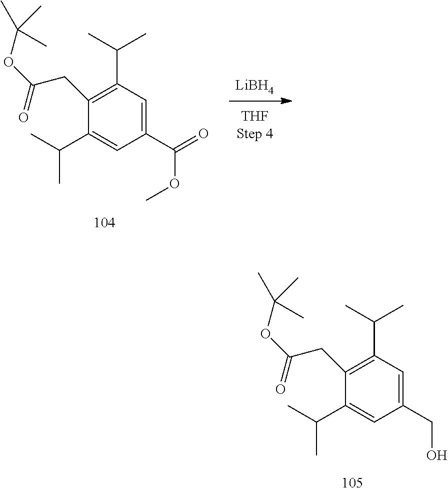 [SMILES: COC(=O)c1cc(C(C)C)c(CC(=O)OC(C)(C)C)c(C(C)C)c1 ; "LiBH4" ; CC(C)c1cc(CO)cc(C(C)C)c1CC(=O)OC(C)(C)C]

Into a 100 mL round bottom flask was placed a solution of methyl 4-(2-tert-butoxy-2-oxoethyl)-3,5-diisopropylbenzoate (2 g, 6.0 mmol) in THE (25 mL). LiBH₄ (264 mg, 12.0 mmol) was added to the mixture at 0° C. in portions, and the mixture was stirred at 0° C. for 1 h. The reaction was quenched with ice-water (20 mL) and extracted with EtOAc (3×100 mL). The combined organic layers were dried over anhydrous Na₂SO₄ and concentrated under vacuum. The residue thus obtained was purified with SiO₂-gel column chromatography and eluted with ethyl acetate/petroleum ether (1:5 to 1:2). This resulted in 1.1 g (60%) of the title compound as a white solid.

¹H NMR (300 MHz, CD3OD-d4) δ 7.81 (s, 2H), 3.91 (s, 2H), 3.82 (s, 2H), 3.27-3.21 (m, 2H), 1.43 (s, 9H), 1.26 (d, J=6.0 Hz, 12H).

### 5. Synthesis of Tert-butyl 2-(2,6-diisopropyl-4-(methoxymethyl)phenyl)acetate

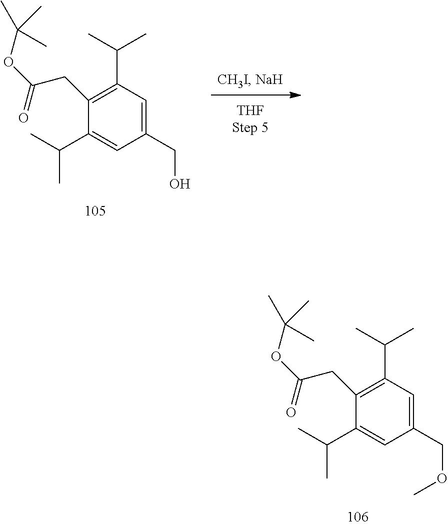

Into a 100 mL round bottom flask was placed a solution of tert-butyl 2-(4-(hydroxymethyl)-2,6-diisopropylphenyl)acetate (1.1 g, 3.6 mmol) in THE (20 mL). NaH (60% wt., 173 mg, 4.3 mmol) was added to the mixture at 0° C. in portions, and the mixture was stirred at 0° C. for 30 min. Mel (1.0 g, 7.2 mmol) was added to the mixture dropwise at 0° C., and the mixture was stirred at RT overnight. The reaction was quenched with ice-water (20 mL) and extracted with EtOAc (3×100 mL). The combined organic layer were dried over Na₂SO₄ and concentrated under vacuum. The residue thus obtained was purified with SiO₂-gel column and eluted with ethyl acetate/petroleum ether (1:10-1:5). This result in 1.1 g (95%) of title compound as a colorless oil.

### 6. Synthesis of 2-(2,6-Diisopropyl-4-(methoxymethyl)phenyl)acetic acid

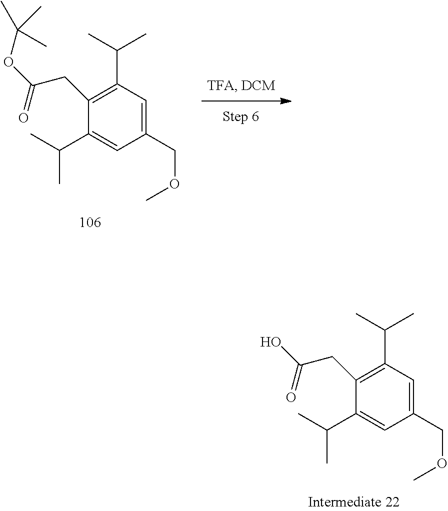

Into a 50-mL round-bottom flask was placed a solution of tert-butyl 2-[4-fluoro-2,6-bis(propan-2-yl)phenyl]acetate (1.1 g, 3.4 mmol) in DCM (10 mL) and TFA (10 mL). The solution was stirred for 3 h at RT and was then concentrated under vacuum. This resulted in 1.0 g (crude) of the title compound as a light yellow solid.

LCMS of 2-(2,6-Diisopropyl-4-(methoxymethyl)phenyl)acetic acid (Method M): 263.2 [M−H]⁻, retention time 0.712 min.

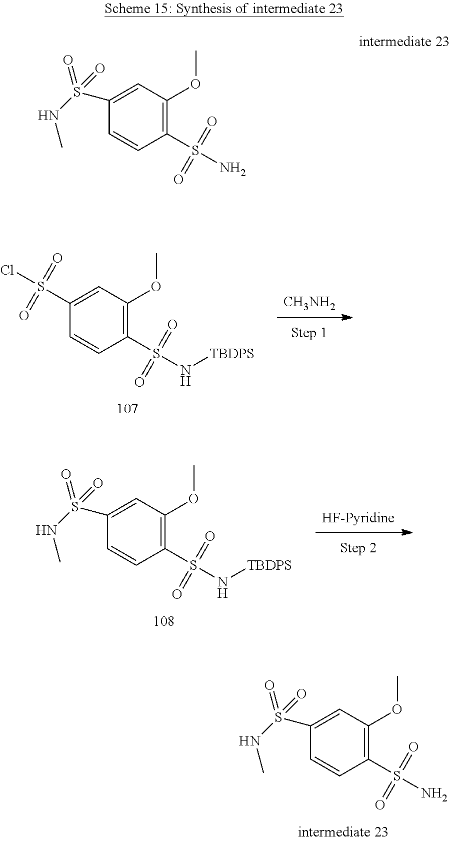

### 1. Synthesis of N1-(tert-butyldiphenylsilyl)-2-methoxy-N4-methylbenzene-1,4-disulfonamide

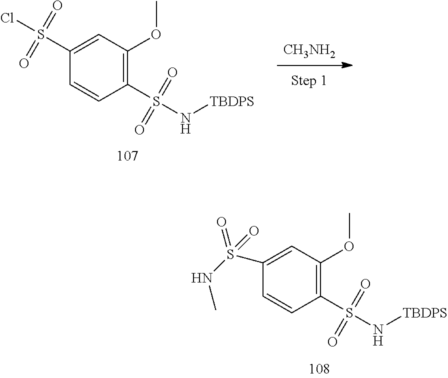

Into a 100 mL round-bottom flask was added methanamine in THE (40 mL, 0.5M) at 0 degrees C. To this stirred solution of MeNH₂ was added crude 4-(N-(tert-butyldiphenylsilyl)sulfamoyl)-3-methoxybenzenesulfonyl chloride (2.8 g) in THE (10 mL) dropwise at 0 degrees C. The resulting mixture was stirred overnight at room temperature. The residue thus obtained was applied onto a silica gel column which was eluted with ethyl acetate/petroleum ether (9:1) to give N1-(tert-butyldiphenylsilyl)-2-methoxy-N4-methylbenzene-1,4-disulfonamide (1.2 g, 52.0% for 2 steps) as yellow solid.

LCMS of N1-(tert-butyldiphenylsilyl)-2-methoxy-N4-methylbenzene-1,4-disulfonamide: 517.1 [M+H]⁺, retention time 1.454 min. Method: YMC Triart-C18, 50*3.0 mm, 1.0 uL injection, 1.0 mL/min flowrate, 90-900 amu scan range, 254 nm UV detection. Mobile phase A: Water (5 mmoL/L NH4HCO3) and Mobile Phase B: MeCN. 10% MPB to 95.0% in 1.1 min, hold at 95% MPB for 0.5 min, 95% MPB to 10% in 0.1 min, then equilibration to 10% MPB for 0.1 min.

### 2. Synthesis of 3-methoxy-N1-methylbenzene-1,4-disulfonamide

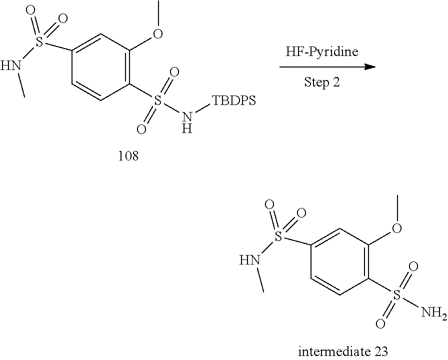

Into a 50-mL round-bottom flask, was placed a solution of N1-(tert-butyldiphenylsilyl)-2-methoxy-N4-methylbenzene-1,4-disulfonamide (218 mg, 0.42 mmol, 1 equiv) in THE (5 mL) and HF-Pyridine (417.9 mg, 4.22 mmol, 10 equiv). The resulting solution was stirred for 1 hr at room temperature, after which it was concentrated. The residue thus obtained was applied onto a silica gel column with MeOH/DCM (1:10) to give 3-methoxy-N1-methylbenzene-1,4-disulfonamide (85.8 mg, 73.0%) as yellow solid.

1H NMR (400 MHz, DMSO-d6) δ 7.95 (d, J=8.0 Hz, 1H), 7.65 (d, J=5.0 Hz, 1H), 7.50 (d, J=1.6 Hz, 1H), 7.47 (dd, J=8.1, 1.6 Hz, 1H), 7.32 (s, 2H), 3.99 (s, 3H), 2.46 (d, J=4.9 Hz, 3H).

LCMS of 3-methoxy-N1-methylbenzene-1,4-disulfonamide: 279.0 [M−H]⁻, retention time 0.688 min. Method: Agilent Poroshell HPH-C18, 50*3.0 mm, 0.8 uL injection, 1.0 mL/min flowrate, 90-900 amu scan range, 254 nm UV detection. Mobile phase A: Water (5 mmoL/L NH4HCO3) and Mobile Phase B: MeCN. 10% MPB to 95.0% in 1.1 min, hold at 95% MPB for 0.5 min, 95% MPB to 10% in 0.1 min, then equilibration to 10% MPB for 0.1 min.

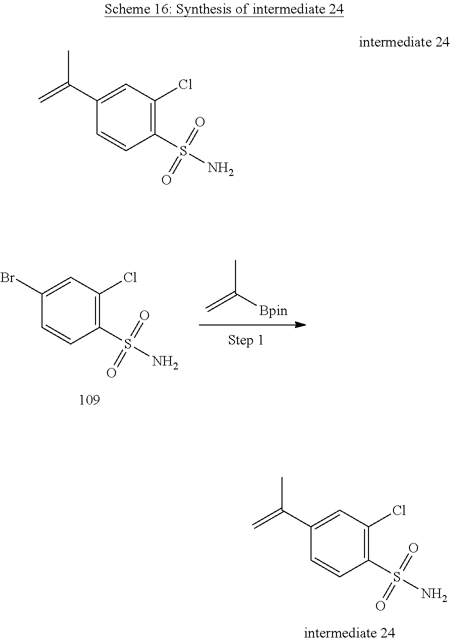

### 1. Synthesis of 2-chloro-4-(prop-1-en-2-yl)benzenesulfonamide

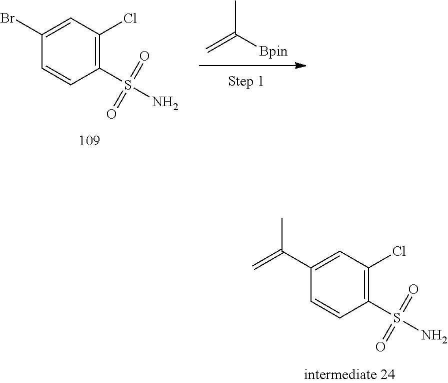

Into a 50-mL round-bottom flask, was placed 4-bromo-2-chlorobenzenesulfonamide (1.0 g, 3.7 mmol, 1.0 equiv) in Dioxane (20 mL)/water (2 mL). Pd(dppf)Cl₂ (540.9 mg, 0.74 mmol, 0.2 equiv) and Cs₂CO₃ (2.4 g, 7.4 mmol, 2.0 equiv) were added to the solution. The resulting solution was stirred for 6 hr at 90 degrees C. The resulting mixture was concentrated and purified with SiO₂-gel column. This resulted in 720 mg (84.2%) of 2-chloro-4-(prop-1-en-2-yl)benzenesulfonamide as a yellow solid.

LCMS of 2-chloro-4-(prop-1-en-2-yl)benzenesulfonamide (Method F): 230.0 [M−H]⁻, retention time 1.160 min.

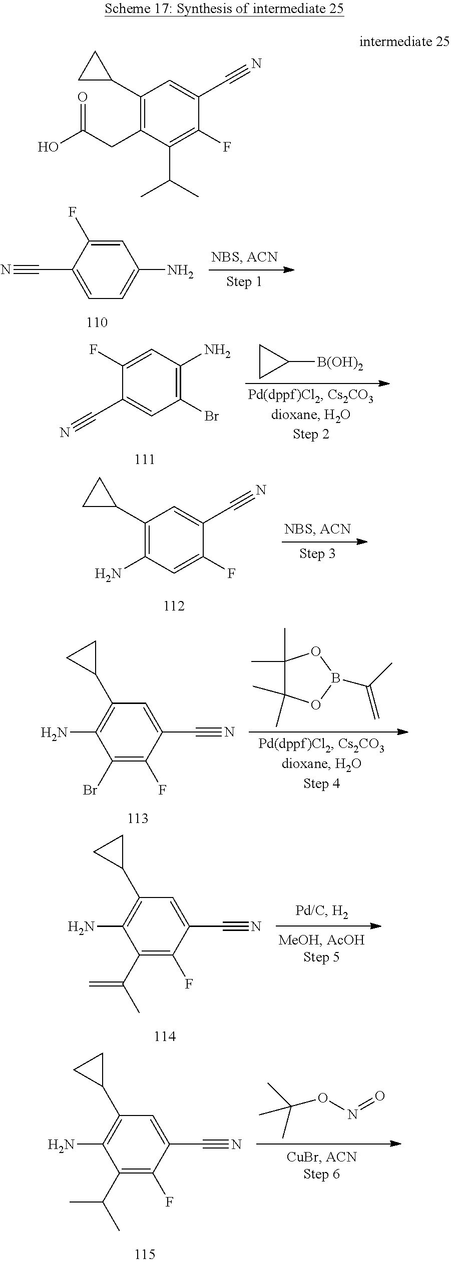

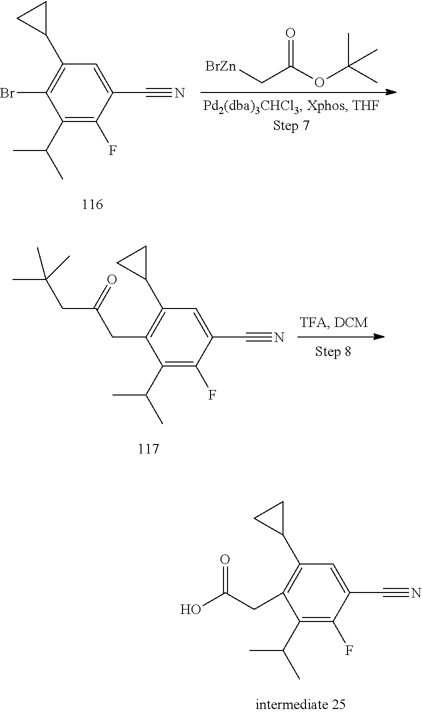

### 1. Synthesis of 4-Amino-5-bromo-2-fluorobenzonitrile

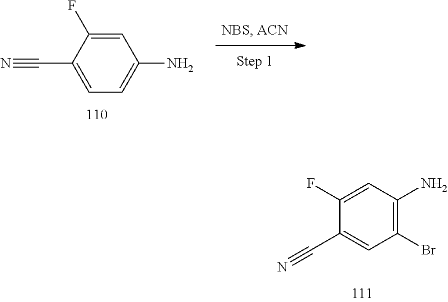 [SMILES: N#Cc1ccc(N)cc1F ; N#Cc1cc(Br)c(N)cc1F]

Into a 250-mL round-bottom flask was placed a solution of 4-amino-2-fluorobenzonitrile (9 g, 66.1 mmol) in ACN (120 mL). Then NBS (12.4 g, 69.7 mmol) was added. The resulting solution was stirred overnight at 80° C. and was then concentrated under vacuum. The residue was applied onto a silica gel column which was eluted with a gradient of ethyl acetate/petroleum ether (1:20 to 1:10). This resulted in 10.9 g (77%) of the title compound as a yellow solid.

MS-ESI: 215.0/217.0 (M+1).

¹H NMR (300 MHz, DMSO-d6) δ 7.89 (d, J=6.0 Hz, 1H), 6.69 (br s, 2H), 6.63 (d, J=12.0 Hz, 1H).

### 2. Synthesis of 4-Amino-5-cyclopropyl-2-fluorobenzonitrile

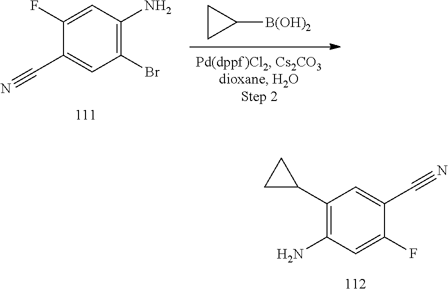 [SMILES: N#Cc1cc(Br)c(N)cc1F ; OB(O)C1CC1 ; N#Cc1cc(C2CC2)c(N)cc1F]

Into a 250-mL round-bottom flask purged with and maintained under nitrogen was placed a solution of 4-amino-5-bromo-2-fluorobenzonitrile (6.37 g, 29.6 mmol) in dioxane (70 mL) and water (10 mL). To the solution were added Cs₂CO₃ (9.7 g, 29.8 mmol), cyclopropylboronic acid (3.8 g, 44.2 mmol), and Pd(dppf)Cl₂ (1.08 g, 1.48 mmol). The resulting solution was stirred overnight at 90° C. and was then concentrated under vacuum. The residue thus obtained was applied onto a silica gel column which was eluted with a gradient of ethyl acetate/petroleum ether (1:10 to 1:5). This resulted in 5.03 g (96%) of the title compound as a yellow solid.

MS-ESI: 177.1 (M+1).

### 3. Synthesis of 4-Amino-3-bromo-5-cyclopropyl-2-fluorobenzonitrile

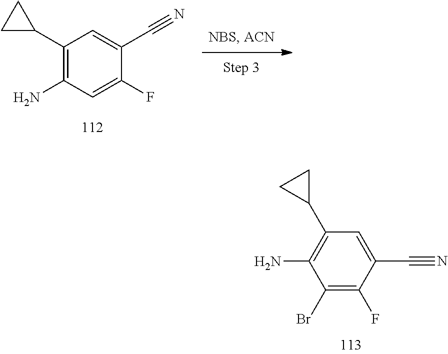 [SMILES: N#Cc1cc(C2CC2)c(N)cc1F ; N#Cc1cc(C2CC2)c(N)c(Br)c1F]

Into a 250-mL round-bottom flask was placed a solution of 4-amino-5-cyclopropyl-2-fluorobenzonitrile (5.03 g, 28.7 mmol) in ACN (50 mL). To the solution was added NBS (5.6 g, 31.5 mmol). The resulting solution was stirred overnight at 80° C. and was then concentrated under vacuum. The residue was applied onto a silica gel column which was eluted with a gradient of ethyl acetate/petroleum ether (1:10 to 1:5). This resulted in 6.972 g (96%) of the title compound as a yellow solid.

LCMS of 4-Amino-3-bromo-5-cyclopropyl-2-fluorobenzonitrile (Method A): 255.0/257.0 [M+H]⁺, retention time 1.361 min.

### 4. Synthesis of 4-Amino-5-cyclopropyl-2-fluoro-3-(prop-1-en-2-yl)benzonitrile

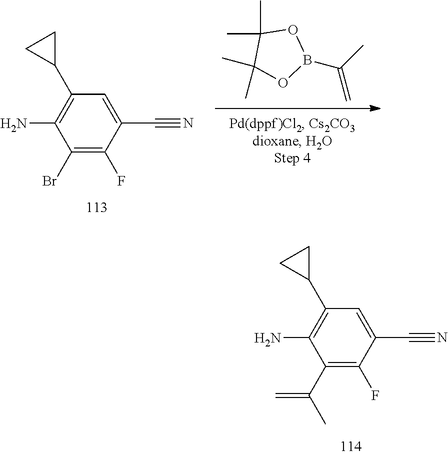 [SMILES: N#Cc1cc(C2CC2)c(N)c(Br)c1F ; C=C(C)B1OC(C)(C)C(C)(C)O1 ; C=C(C)c1c(N)c(C2CC2)cc(C#N)c1F]

Into a 250-mL round-bottom flask purged with and maintained under nitrogen was placed a solution of 4-amino-3-bromo-5-cyclopropyl-2-fluorobenzonitrile (6.972 g, 27.33 mmol) in 1,4-dioxane (120 mL) and water (20 mL). To the solution were added 4,4,5,5-tetramethyl-2-(prop-1-en-2-yl)-1,3,2-dioxaborolane (6.9 g, 41.00 mmol), Cs₂CO₃ (13.4 g, 41.00 mmol), and Pd(dppf)Cl₂ (0.4 g, 0.55 mmol). The resulting solution was stirred overnight at 80° C. and was then concentrated under vacuum. The residue thus obtained was applied onto a silica gel column and eluted with a gradient of ethyl acetate/petroleum ether (1:10 to 1:5). This resulted in 4.73 g (80%) of the title compound as a yellow solid.

LCMS of 4-amino-5-cyclopropyl-2-fluoro-3-(prop-i-en-2-yl)benzonitrile (Method A): 217.2 [M+H]⁺, retention time 1.395 min.

### 5. Synthesis of 4-Amino-5-cyclopropyl-2-fluoro-3-isopropylbenzonitrile

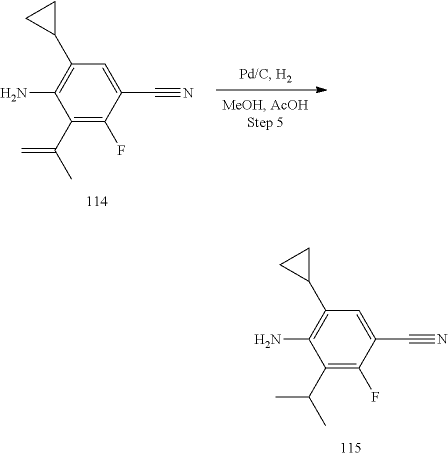 [SMILES: C=C(C)c1c(N)c(C2CC2)cc(C#N)c1F ; CC(C)c1c(N)c(C2CC2)cc(C#N)c1F]

Into a 250-mL round-bottom flask was placed a solution of 4-amino-5-cyclopropyl-2-fluoro-3-(prop-1-en-2-yl)benzonitrile (4.73 g, 21.97 mmol) in MeOH (100 mL). To the solution was added AcOH (0.5 mL). Then Pd/C (10% wt., 500 mg) was added. The flask was evacuated and filled three times with hydrogen. The resulting solution was stirred for 4 h at 40° C. under an atmosphere of hydrogen. The solids were filtered out. The filtrate was concentrated under vacuum. This resulted in 4.71 g (99%) of the title compound as a light yellow solid.

LCMS of 4-Amino-5-cyclopropyl-2-fluoro-3-isopropylbenzonitrile (Method A): 219.1 [M+H]⁺, retention time 1.412 min.

### 6. Synthesis of 4-bromo-5-cyclopropyl-2-fluoro-3-isopropylbenzonitrile

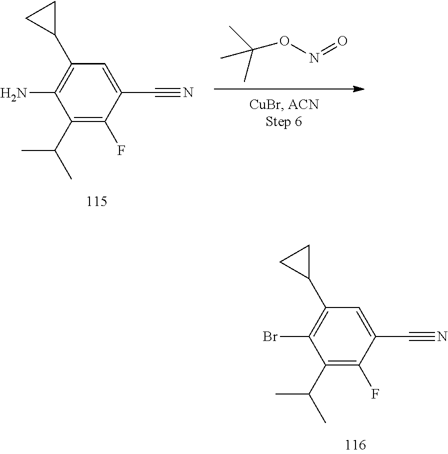

Into a 500-mL round-bottom flask purged with and maintained under nitrogen was placed 4-amino-5-cyclopropyl-2-fluoro-3-isopropylbenzonitrile (3.9 g, 18 mmol), ACN (150 mL), and CuBr (4 g, 27 mmol). This was followed by the addition of tert-butyl nitrite (2.8 g, 27 mmol) dropwise with stirring at 0° C. The resulting solution was stirred for 3 h at 60° C. and was then concentrated under vacuum. The residue was applied onto a silica gel column eluted with petroleum ether. This resulted in 3.2 g (64%) of the title compound as yellow oil.

### 7. Synthesis of tert-butyl 2-(4-cyano-6-cyclopropyl-3-fluoro-2-isopropylphenyl)acetate

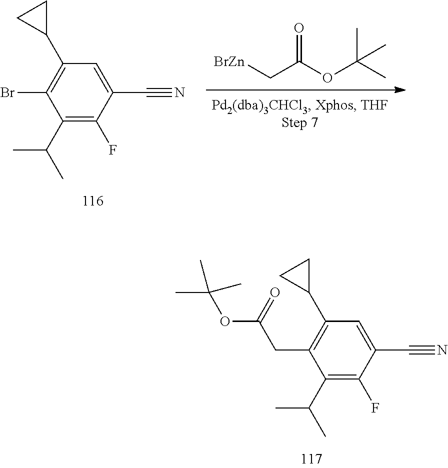

Into a 250-mL 3-necked round-bottom flask purged with and maintained under nitrogen was placed 4-bromo-5-cyclopropyl-2-fluoro-3-isopropylbenzonitrile (3.2 g, 11.6 mmol), THE (150 mL), X-phos (553 mg, 1.16 mmol), and Pd₂(dba)₃CHCl₃ (600 mg, 0.58 mmol). The resulting solution was stirred for 0.5 h at RT. Then to the above mixture tert-butyl 2-(bromozincio)acetate (6.0 g, 23.04 mmol) was added. The resulting solution was stirred for 5 h at 70° C., after which the reaction was quenched by the addition of 100 mL of NH₄ Cl (sat.). The resulting mixture was extracted with 3×100 mL of ethyl acetate. The organic layers were combined and concentrated under vacuum. The residue was applied onto a silica gel column which was eluted with ethyl acetate/petroleum ether (1:100 to 3:97). This resulted in 1.8 g (50%) of the title compound as yellow oil.

LCMS of tert-butyl 2-(4-cyano-6-cyclopropyl-3-fluoro-2-isopropylphenyl)acetate (Method A): 318.3 [M+H]⁺, retention time 1.605 min.

### 8. Synthesis of 2-(4-cyano-6-cyclopropyl-3-fluoro-2-isopropylphenyl)acetic acid

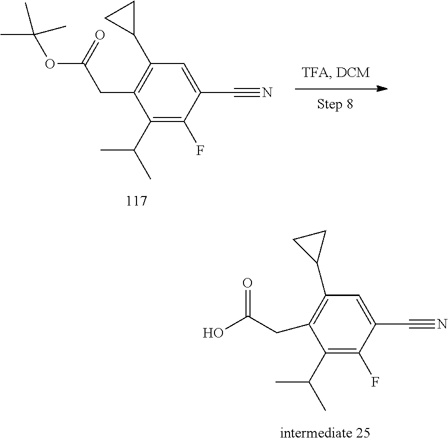 [SMILES: CC(C)c1c(F)c(C#N)cc(C2CC2)c1CC(=O)OC(C)(C)C ; CC(C)c1c(F)c(C#N)cc(C2CC2)c1CC(=O)O]

Into a 50-mL round-bottom flask was placed tert-butyl 2-(4-cyano-6-cyclopropyl-3-fluoro-2-isopropylphenyl)acetate (1.8 g, 5.6 mmol), DCM (10 mL), and TFA (10 mL). The resulting solution was stirred for 3 h at RT and was then concentrated under vacuum. The crude product was treated with 100 mL of NaOH (4 N) and extracted with 3×50 mL of DCM to remove impurities. The pH value of aqueous phase was adjusted to 2 with HCl (4 N), and the aqueous phase was then extracted with 3×100 mL of DCM. The combined organic layers were dried over anhydrous Na₂SO₄ and concentrated under vacuum. This resulted in 1.2 g (85%) of the title compound as a light yellow solid.

LCMS of 2-(4-cyano-6-cyclopropyl-3-fluoro-2-isopropylphenyl)acetic acid (Method N): 260.1 [M−H]⁻, retention time 0.710 min.

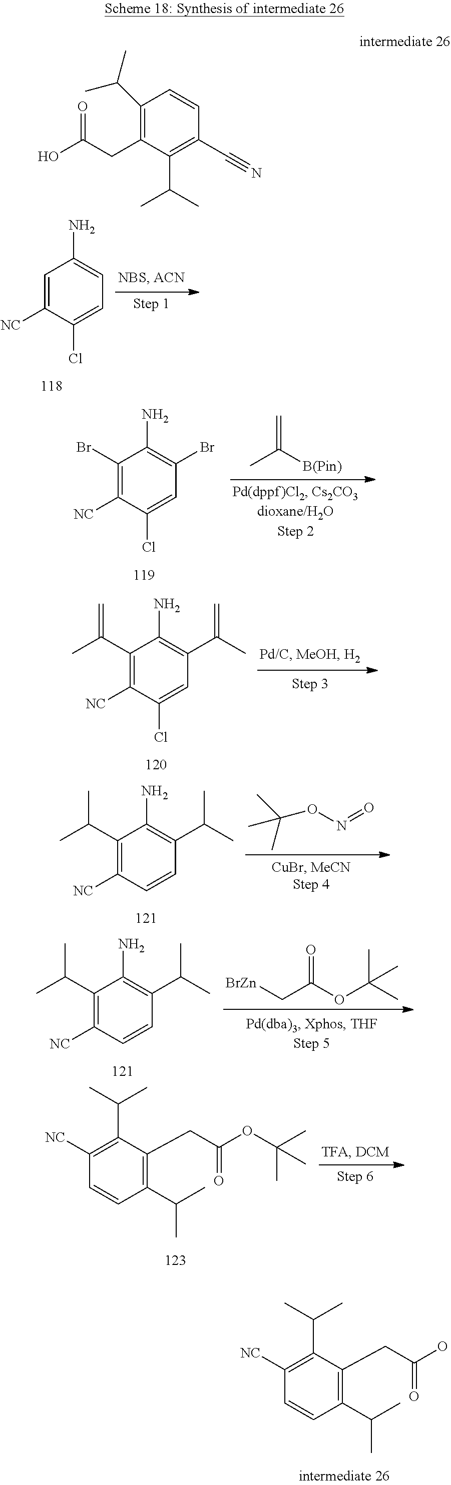

### 1. Synthesis of 3-amino-2,4-dibromo-6-chlorobenzonitrile

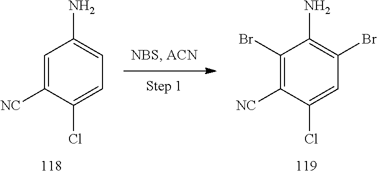 [SMILES: N#Cc1cc(N)ccc1Cl ; N#Cc1c(Cl)cc(Br)c(N)c1Br]

Into a 500-mL round-bottom flask, was placed 5-amino-2-chlorobenzonitrile (10 g, 1 equiv), ACN (200 mL), and NBS (29 g, 1.5 equiv). The resulting solution was stirred for 14 hr at room temperature. The resulting mixture was concentrated. The residue thus obtained was applied onto a silica gel column with ethyl acetate/petroleum ether (1:15 to 1:5). This resulted in 18 g of 3-amino-2,4-dibromo-6-chlorobenzonitrile as a yellow solid.

LCMS of 3-amino-2,4-dibromo-6-chlorobenzonitrile (Method I): 309, 311, 313 [M+H]⁺, retention time 1.083 min.

### 2. Synthesis of 3-amino-6-chloro-2,4-bis(prop-1-en-2-yl)benzonitrile

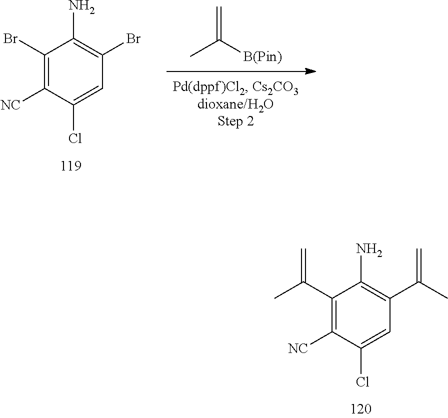

Into a 500-mL round-bottom flask purged with and maintained under an inert atmosphere of nitrogen, was placed 3-amino-2,4-dibromo-6-chlorobenzonitrile (15 g, 48 mmol, 1 equiv), dioxane (200 mL), 2-(tetramethyl-1,3,2-dioxaborolan-2-yl)prop-2-en-1-ylium (18.5 g, 111 mmol, 2.2 equiv), Cs₂CO₃ (47 g, 3 equiv), H₂O (20 mL), and Pd(dppf)Cl₂ (1.5 g). The resulting solution was stirred for 14 hr at 100 degrees C. in an oil bath. The resulting mixture was concentrated. The residue thus obtained was applied onto a silica gel column with ethyl acetate/petroleum ether (1:0 to 1:25). This resulted in 10 g of 3-amino-6-chloro-2,4-bis(prop-1-en-2-yl)benzonitrile as brown oil.

LCMS of 3-amino-6-chloro-2,4-di(prop-1-en-2-yl)benzonitrile (Method A): 233, 235 [M+H]⁺, retention time 1.465 min.

### 3. Synthesis of 3-amino-2,4-bis(propan-2-yl)benzonitrile

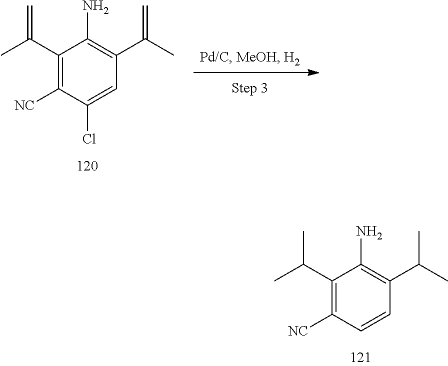 [SMILES: C=C(C)c1cc(Cl)c(C#N)c(C(=C)C)c1N ; CC(C)c1ccc(C#N)c(C(C)C)c1N]

Into a 500-mL round-bottom flask purged with and maintained under an inert atmosphere of nitrogen, was placed 3-amino-6-chloro-2,4-bis(prop-1-en-2-yl)benzonitrile (10 g, 43 mmol), methanol (50 mL), and Pd/C (2 g). The resulting solution was hydrogenated with H2 and stirred for 14 hr at room temperature in a water bath. The solids were filtered out. The filtrate was concentrated. This resulted in 8 g of 3-amino-2,4-bis(propan-2-yl)benzonitrile as brown oil.

LCMS of 3-amino-2,4-diisopropylbenzonitrile (Method J): 203 [M+H]⁺, retention time 1.400 min.

### 4. Synthesis of 3-bromo-2,4-bis(propan-2-yl)benzonitrile

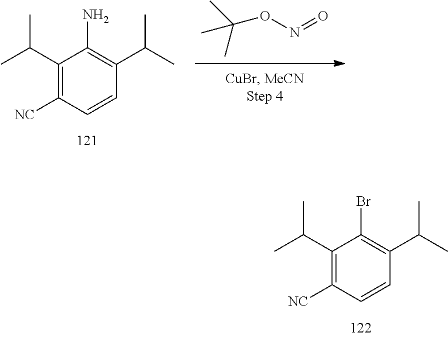

Into a 250-mL round-bottom flask, was placed 3-amino-2,4-bis(propan-2-yl)benzonitrile (8 g, 39.55 mmol, 1 equiv), ACN (150 mL), CuBr (11.3 g, 79.09 mmol, 2 equiv), and tert-butyl nitrite (8.2 g, 79.09 mmol, 2 equiv). The resulting solution was stirred for 3 hr at 60 degrees C. in an oil bath. The resulting mixture was concentrated. The residue thus obtained was applied onto a silica gel column which was eluted with ethyl acetate/petroleum ether (1:50). This resulted in 4.2 g (39.90%) of 3-bromo-2,4-bis(propan-2-yl)benzonitrile as purple oil.

### 5. Synthesis of tert-butyl 2-[3-cyano-2,6-bis(propan-2-yl)phenyl]acetate

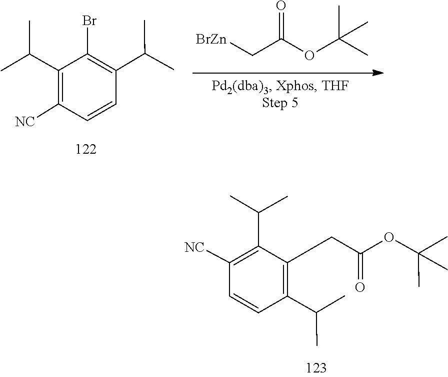

Into a 250-mL round-bottom flask, was placed 3-bromo-2,4-bis(propan-2-yl)benzonitrile (3.1 g, 11.65 mmol, 1 equiv), Xphos (555.2 mg, 1.16 mmol, 0.1 equiv), Pd₂(dba)₃ (533.2 mg, 0.58 mmol, 0.05 equiv), THE (100 mL), and tert-butyl 2-(bromozincio)acetate (7.6 g, 29.12 mmol, 2.5 equiv) under a nitrogen atmosphere. The resulting solution was stirred for 3 hr at 65 degrees C. in an oil bath, after which it was concentrated. The residue thus obtained was applied onto a silica gel column which was eluted with ethyl acetate/petroleum ether (1:50). This resulted in 3.0 g (85.46%) of tert-butyl 2-[3-cyano-2,6-bis(propan-2-yl)phenyl]acetate as purple oil.

### 6. Synthesis of 2-[3-cyano-2,6-bis(propan-2-yl)phenyl]acetic acid

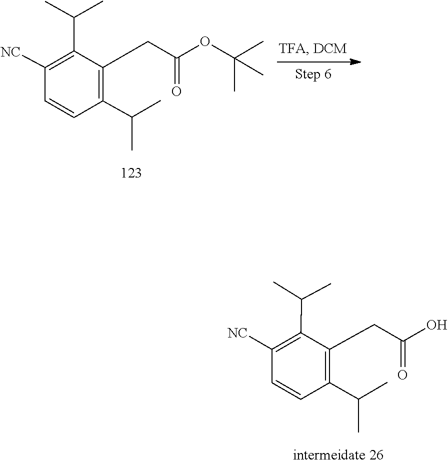 [SMILES: CC(C)c1ccc(C#N)c(C(C)C)c1CC(=O)OC(C)(C)C ; CC(C)c1ccc(C#N)c(C(C)C)c1CC(=O)O]

Into a 100-mL round-bottom flask, was placed tert-butyl 2-[3-cyano-2,6-bis(propan-2-yl)phenyl]acetate (3.4 g, 11.28 mmol, 1 equiv), DCM (15 mL), and TFA (15 mL). The resulting solution was stirred for 3 hr at room temperature. The resulting mixture was concentrated. The residue thus obtained was applied onto a silica gel column with ethyl acetate/petroleum ether (1:3). This resulted in 2.6 g (93.96%) of 2-[3-cyano-2,6-bis(propan-2-yl)phenyl]acetic acid as a light yellow solid.

LCMS of 2-(3-cyano-2,6-diisopropylphenyl)acetic acid (Method M): 244 [M−H]⁻, retention time 0.674 min.

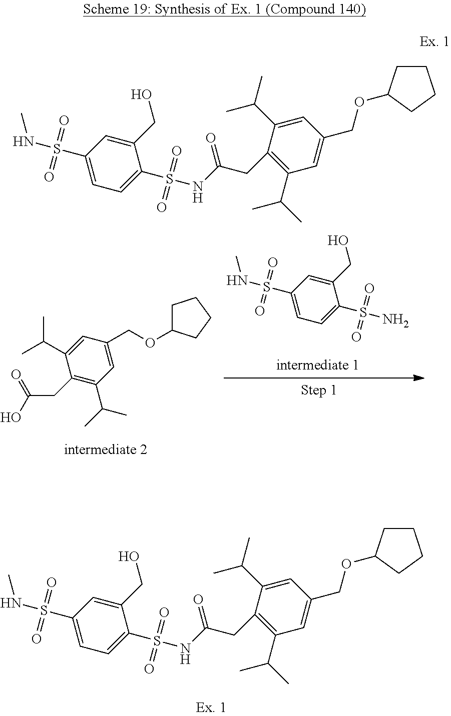 [SMILES: CNS(=O)(=O)c1ccc(S(=O)(=O)NC(=O)Cc2c(C(C)C)cc(COC3CCCC3)cc2C(C)C)c(CO)c1 ; CC(C)c1cc(COC2CCCC2)cc(C(C)C)c1CC(=O)O ; CNS(=O)(=O)c1ccc(S(N)(=O)=O)c(CO)c1 ; CNS(=O)(=O)c1ccc(S(=O)(=O)NC(=O)Cc2c(C(C)C)cc(COC3CCCC3)cc2C(C)C)c(CO)c1]

### 1. Synthesis of 2-[4-[(cyclopentyloxy)methyl]-2,6-bis(propan-2-yl)phenyl]-N-[2-(hydroxymethyl)-4-(methylsulfamoyl)benzenesulfonyl]acetamide

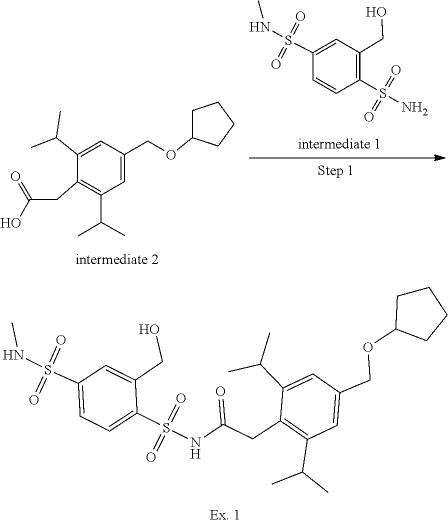 [SMILES: CC(C)c1cc(COC2CCCC2)cc(C(C)C)c1CC(=O)O ; CNS(=O)(=O)c1ccc(S(N)(=O)=O)c(CO)c1 ; CNS(=O)(=O)c1ccc(S(=O)(=O)NC(=O)Cc2c(C(C)C)cc(COC3CCCC3)cc2C(C)C)c(CO)c1]

Into a 50 mL round-bottom flask were added 2-[4-[(cyclopentyloxy)methyl]-2,6-bis(propan-2-yl)phenyl]acetic acid (140 mg, 0.44 mmol, 1 equiv) and DCM (5 mL) at room temperature. To a stirred solution of 2-[4-[(cyclopentyloxy)methyl]-2,6-bis(propan-2-yl)phenyl]acetic acid(140 mg, 0.44 mmol, 1 equiv) in DCM (5 mL) was added DMF(0.01 mL) and oxalic dichloride (0.4 mL) in one portion at room temperature. The resulting mixture was stirred for 1 h at room temperature. The resulting mixture was concentrated under reduced pressure. The crude product, 2-[4-[(cyclopentyloxy)methyl]-2,6-bis(propan-2-yl)phenyl]acetyl chloride, was used directly without further purification.

Into a 50 mL round-bottom flask were added 3-(hydroxymethyl)-N1-methylbenzene-1,4-disulfonamide (83.2 mg, 0.30 mmol, 1.00 equiv) and THE (5 mL) at 0 degrees C. To a stirred mixture of 3-(hydroxymethyl)-N1-methylbenzene-1,4-disulfonamide(83.2 mg, 0.30 mmol, 1.00 equiv) in THF(5 mL) was added NaH (14.2 mg, 0.59 mmol, 2.00 equiv) in one portion at 0 degrees C. under nitrogen atmosphere. The resulting mixture was stirred for 30 min at 0 degrees C. under nitrogen atmosphere. Then 2-[4-[(cyclopentyloxy)methyl]-2,6-bis(propan-2-yl)phenyl]acetyl chloride (100 mg, 0.30 mmol, 1 equiv) in THF(5 mL) was added to the above mentioned mixture. The resulting mixture was stirred for 4 h at room temperature. The reaction was quenched with water (1 mL) at 0 degrees C., after which it was concentrated under reduced pressure. The crude product (600 mg) was purified by Prep-HPLC with the following conditions (Column: XBridge Prep C18 OBD Column, 5 um, 19*150 mm; Mobile Phase A:Water(10MMOL/L NH4HCO3), Mobile Phase B: ACN; Flow rate: 25 mL/min; Gradient: 20% B to 55% B in 8 min; 254/210 nm; Rt: 7.35; 9.6 min) to afford 2-[4-[(cyclopentyloxy)methyl]-2,6-bis(propan-2-yl)phenyl]-N-[2-(hydroxymethyl)-4-(methylsulfamoyl)benzenesulfonyl]acetamide(45.0 mg, 25.69%) as a white solid.

LC-MS-2-[4-[(cyclopentyloxy)methyl]-2,6-bis(propan-2-yl)phenyl]-N-[2-(hydroxymethyl)-4-(methylsulfamoyl)benzenesulfonyl]acetamide: (ES, m/z): 581.3[M+1], retention time: 1.458. Method: Kromasil Eternity-2.5-C18, 3×50 mm, 2.5 um column, 5.0 uL injection, 1.0 mL/min flow rate, 90-900 amnu scan range, 190-400 nm UV range, 10% MPB to 95% in 2.1 min, hold at 95% MPB for 0.6 min gradient with ACN and water (0.5% N₄HCO₃), 3 minute total run time.

H-NMR-2-[4-[(cyclopentyloxy)methyl]-2,6-bis(propan-2-yl)phenyl]-N-[2-(hydroxymethyl)-4-(methylsulfamoyl)benzenesulfonyl]acetamide: (300 MHz, DMSO-d) δ 8.10 (s, 1H), 7.976-7.948 (d, J=8.4 Hz, 1H), 7.675-7.646 (d, J=8.7 Hz, 1H), 7.192 (s, 2H), 4.950 (s, 2H), 4.276 (s, 2H), 3.880 (s, 1H), 3.569 (s, 2H), 2.951-2.885 (m, 2H), 2.362 (s, 3H), 1.581-1.392 (m, 8H), 0.968-0.946 (m, 12H).

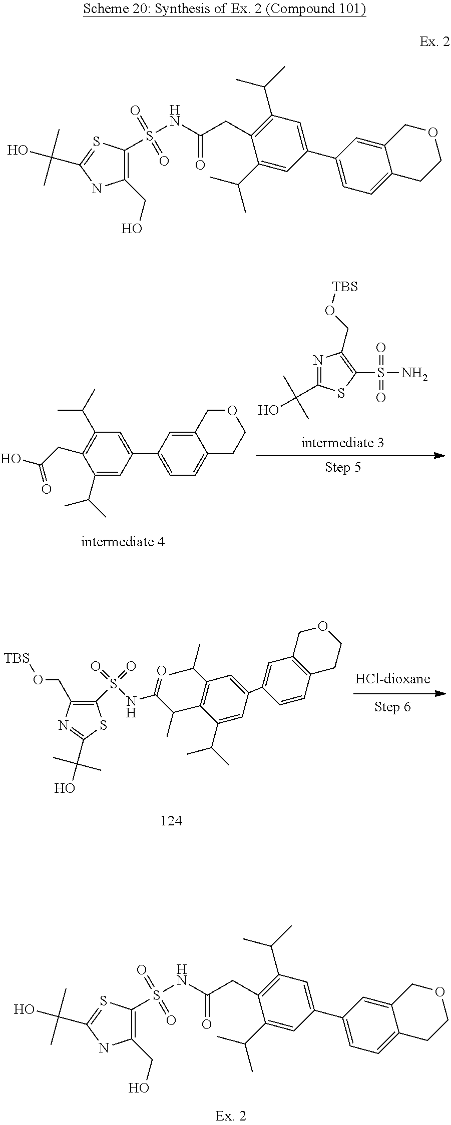

### 1. Synthesis of N-((4-(((tert-butyldimethylsilyl)oxy)methyl)-2-(2-hydroxypropan-2-yl)thiazol-5-yl)sulfonyl)-2-(4-(isochroman-7-yl)-2,6-diisopropylphenyl)acetamide

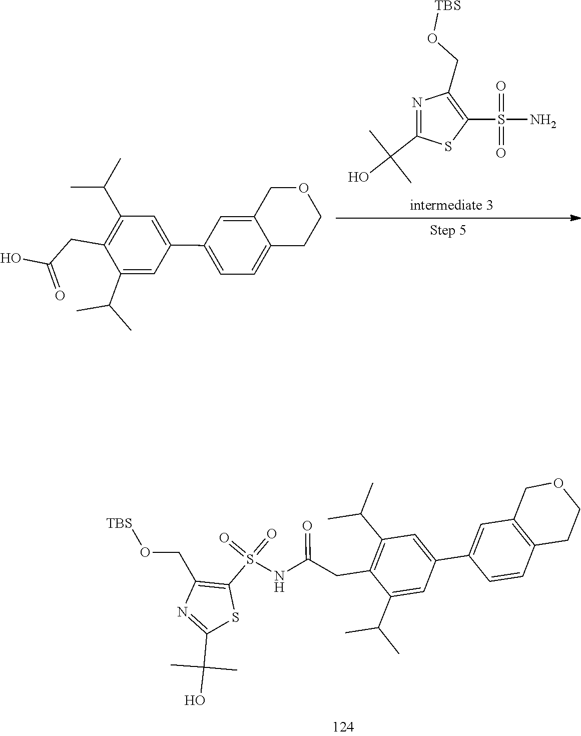

Into a 25-mL round-bottom flask purged with and maintained under an inert atmosphere of nitrogen, was placed 2-[4-(3,4-dihydro-1H-2-benzopyran-7-yl)-2,6-bis(propan-2-yl)phenyl]acetic acid (120 mg, 0.340 mmol, 1 equiv), THE (5 mL), (COCl)₂ (129.64 mg, 1.021 mmol, 3.0 equiv), and DMF (2.49 mg, 0.034 mmol, 0.1 equiv). The reaction mixture was stirred for 30 min at room temperature, after which it was concentrated. This resulted in 100 mg (79.19%) of 2-[4-(3,4-dihydro-1H-2-benzopyran-7-yl)-2,6-bis(propan-2-yl)phenyl]acetyl chloride as yellow oil.

Into a 20-mL sealed tube purged with and maintained under an inert atmosphere of nitrogen, was placed 2-[4-(3,4-dihydro-1H-2-benzopyran-7-yl)-2,6-bis(propan-2-yl)phenyl]acetyl chloride (100 mg, 0.270 mmol, 1 equiv), DCM (5 mL), DIEA (104.53 mg, 0.809 mmol, 3.0 equiv), and N-(tert-butyldimethylsilyl)-4-[[(tert-butyldimethylsilyl)oxy]methyl]-2-(2-hydroxypropan-2-yl)-1,3-thiazole-5-sulfonamide (194.45 mg, 0.404 mmol, 1.5 equiv). The resulting solution was stirred for 30 min at room temperature. The resulting mixture was concentrated. This resulted in 100 mg (52.99%) of N-((4-(((tert-butyldimethylsilyl)oxy)methyl)-2-(2-hydroxypropan-2-yl)thiazol-5-yl)sulfonyl)-2-(4-(isochroman-7-yl)-2,6-diisopropylphenyl)acetamide as yellow oil.

### 2. Synthesis of 2-[4-(3,4-dihydro-1H-2-benzopyran-7-yl)-2,6-bis(propan-2-yl)phenyl]-N-[[4-(hydroxymethyl)-2-(2-hydroxypropan-2-yl)-1,3-thiazol-5-yl]sulfonyl]acetamide

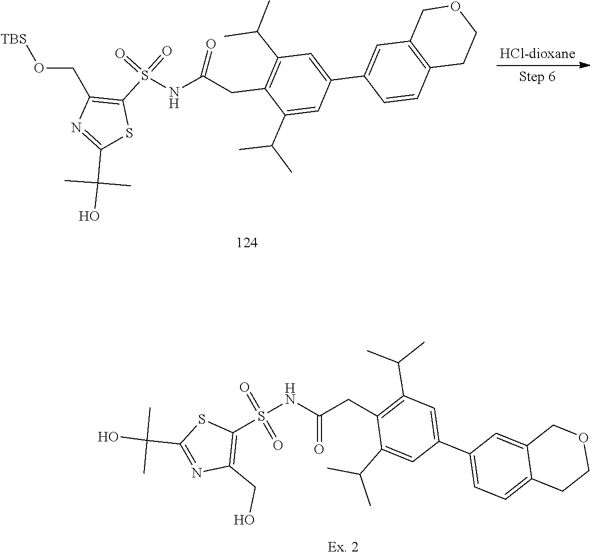

Into a 25-mL round-bottom flask purged with and maintained under an inert atmosphere of nitrogen, was placed N-((4-(((tert-butyldimethylsilyl)oxy)methyl)-2-(2-hydroxypropan-2-yl)thiazol-5-yl)sulfonyl)-2-(4-(isochroman-7-yl)-2,6-diisopropylphenyl)acetamide (110 mg, 0.16 mmol, 1 equiv), THE (2.0 mL, 27.74 mmol, 157.10 equiv), HF-Pyridine (155.7 mg, 1.57 mmol, 10.00 equiv). The resulting solution was stirred for 40 min at room temperature. The resulting solution was diluted with 10 mL of water. The pH value of the solution was adjusted to 7 with NaOH (4 mol/L). The resulting mixture was concentrated and filtered. The crude product (was purified by Chiral-Prep-HPLC with the following conditions: Column: XBridge Prep C18 OBD Column, 5 um, 19*150 mm; mobile Phase A:Water (10MMOL/L NH₄HCO₃), Mobile Phase B: ACN; Flow rate: 25 mL/min; Gradient: 8% B to 60% B in 7.5 min; 254/210 nm; Rt: 6.77 min. This resulted in 25.2 mg (27.33%) of 2-[4-(3,4-dihydro-1H-2-benzopyran-7-yl)-2,6-bis(propan-2-yl)phenyl]-N-[[4-(hydroxymethyl)-2-(2-hydroxypropan-2-yl)-1,3-thiazol-5-yl]sulfonyl]acetamide as a white solid.

LC-MS-2-[4-(3,4-dihydro-1H-2-benzopyran-7-yl)-2,6-bis(propan-2-yl)phenyl]-N-[[4-(hydroxymethyl)-2-(2-hydroxypropan-2-yl)-1,3-thiazol-5-yl]sulfonyl]acetamide(Method D): (ES, m/z): 587.2[M+1]⁺, retention time: 1.254.

H-NMR-2-[4-(3,4-dihydro-1H-2-benzopyran-7-yl)-2,6-bis(propan-2-yl)phenyl]-N-[[4-(hydroxymethyl)-2-(2-hydroxypropan-2-yl)-1,3-thiazol-5-yl]sulfonyl]acetamide: (DMSO, ppm): δ 7.52 (m 1H), 7.33-7.09 (m, 4H), 4.72 (d, J=20.9 Hz, 3H), 3.90 (d, J=5.7 Hz, 2H), 3.76 (m, 1H), 2.43 (s, 1H), 2.81 (s, 2H), 2.31 (m, 1H), 1.47 (s, 6H), 1.10 (d, J=6.7 Hz, 11H).

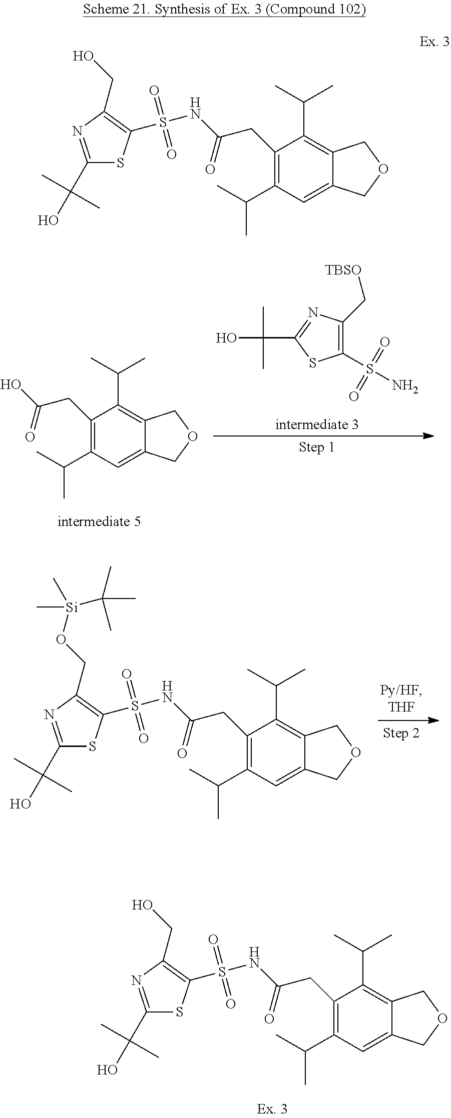 [SMILES: CC(C)c1cc2c(c(C(C)C)c1CC(=O)NS(=O)(=O)c1sc(C(C)(C)O)nc1CO)COC2 ; CC(C)c1cc2c(c(C(C)C)c1CC(=O)O)COC2 ; CC(C)(O)c1nc(CO[Si](C)(C)C(C)(C)C)c(S(N)(=O)=O)s1 ; CC(C)c1cc2c(c(C(C)C)c1CC(=O)NS(=O)(=O)c1sc(C(C)(C)O)nc1CO[Si](C)(C)C(C)(C)C)COC2 ; CC(C)c1cc2c(c(C(C)C)c1CC(=O)NS(=O)(=O)c1sc(C(C)(C)O)nc1CO)COC2]

### 1. Synthesis of 2-[4,6-bis(propan-2-yl)-1,3-dihydro-2-benzofuran-5-yl]-N-[(4-[[(tert-butyldimethylsilyl)oxy]methyl]-2-(2-hydroxypropan-2-yl)-1,3-thiazol-5-yl)sulfonyl]acetamide

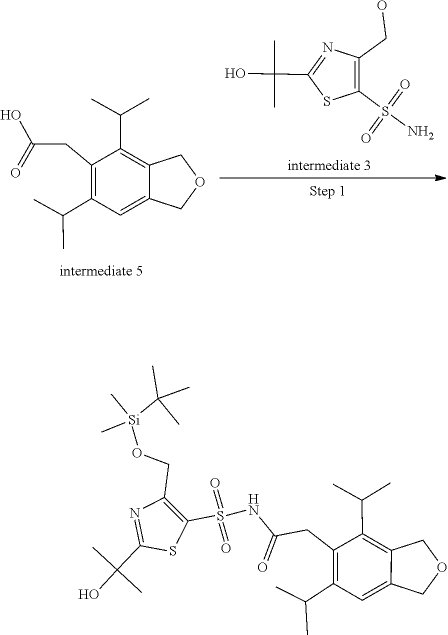

Into a 25-mL round-bottom flask purged with and maintained under an inert atmosphere of nitrogen, was placed 2-[4,6-bis(propan-2-yl)-1,3-dihydro-2-benzofuran-5-yl]acetic acid (120 mg, 0.457 mmol, 1 equiv), THF, (COCl)₂ (174.17 mg, 1.372 mmol, 3.0 equiv), and DMF (3.34 mg, 0.046 mmol, 0.1 equiv). The resulting solution was stirred for 30 min at room temperature. The resulting mixture was concentrated. This resulted in 100 mg (77.86%) of 2-[4,6-bis(propan-2-yl)-1,3-dihydro-2-benzofuran-5-yl]acetyl chloride as yellow oil.

Into a 20-mL sealed tube purged and maintained with an inert atmosphere of nitrogen, was placed 2-[4,6-bis(propan-2-yl)-1,3-dihydro-2-benzofuran-5-yl]acetyl chloride (100 mg, 0.356 mmol, 1 equiv), DCM (5 mL), DIEA (138.09 mg, 1.068 mmol, 3.0 equiv), 4-[[(tert-butyldimethylsilyl)oxy]methyl]-2-(2-hydroxypropan-2-yl)-1,3-thiazole-5-sulfonamide (195.82 mg, 0.534 mmol, 1.5 equiv). The resulting solution was stirred for 30 min at room temperature. The resulting mixture was concentrated. This resulted in 100 mg (45.96%) of 2-[4,6-bis(propan-2-yl)-1,3-dihydro-2-benzofuran-5-yl]-N-[(4-[[(tert-butyldimethylsilyl)oxy]methyl]-2-(2-hydroxypropan-2-yl)-1,3-thiazol-5-yl)sulfonyl]acetamide as yellow oil.

### 2. Synthesis of 2-[4,6-bis(propan-2-yl)-1,3-dihydro-2-benzofuran-5-yl]-N-[[4-(hydroxymethyl)-2-(2-hydroxypropan-2-yl)-1,3-thiazol-5-yl]sulfonyl]acetamide

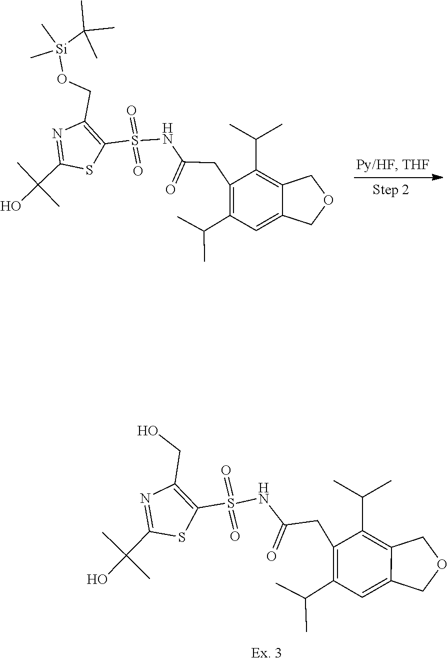 [SMILES: CC(C)c1cc2c(c(C(C)C)c1CC(=O)NS(=O)(=O)c1sc(C(C)(C)O)nc1CO[Si](C)(C)C(C)(C)C)COC2 ; CC(C)c1cc2c(c(C(C)C)c1CC(=O)NS(=O)(=O)c1sc(C(C)(C)O)nc1CO)COC2]

Into a 25-mL round-bottom flask purged with and maintained under an inert atmosphere of nitrogen, was placed 2-[4,6-bis(propan-2-yl)-1,3-dihydro-2-benzofuran-5-yl]-N-[(4-[[(tert-butyldimethylsilyl)oxy]methyl]-2-(2-hydroxypropan-2-yl)-1,3-thiazol-5-yl)sulfonyl]acetamide (70 mg, 0.11 mmol, 1 equiv), THE (2 mL, 0.03 mmol, 0.24 equiv), and HF Pyridine (113.6 mg, 1.15 mmol, 10.00 equiv). The resulting solution was stirred for 1 hr at room temperature. The resulting solution was diluted with 10 mL of water. The pH value of the solution was adjusted to 7 with NaOH (4 mol/L). The solids were filtered out. The filtrate was concentrated. The crude product was purified by Chiral-Prep-HPLC with the following conditions:Column: XBridge Prep C18 OBD Column, 5 um, 19*150 mm; Mobile Phase A: Water (10MMOL/L NH4HCO3), Mobile Phase B: ACN; Flow rate: 25 mL/min; Gradient: 8% B to 60% B in 7.5 min; 254/210 nm; Rt: 6.77 min. This resulted in 8.3 mg (14.59%) of 2-[4,6-bis(propan-2-yl)-1,3-dihydro-2-benzofuran-5-yl]-N-[[4-(hydroxymethyl)-2-(2-hydroxypropan-2-yl)-1,3-thiazol-5-yl]sulfonyl]acetamide as a white solid.

LC-MS-2-[4,6-bis(propan-2-yl)-1,3-dihydro-2-benzofuran-5-yl]-N-[[4-(hydroxymethyl)-2-(2-hydroxypropan-2-yl)-1,3-thiazol-5-yl]sulfonyl]acetamide: (ES, m/z): 497.2 [M+H]⁺, retention time: 1.684. Method: Shim-pack XR-ODS, 50*3.0 mm, 0.8 uL injection, 1.2 mL/min flowrate, 90-900 amu scan range, 254 nm UV detection. Mobile phase A: Water (5 mmoL/L NH4HCO3) and Mobile Phase B: MeCN. 10% MPB to 95.0% in 2.0 min, hold at 95% MPB for 0.7 min, 95% MPB to 10% in 0.05 min, then equilibration to 10% MPB for 0.25 min.

H-NMR-2-[4,6-bis(propan-2-yl)-1,3-dihydro-2-benzofuran-5-yl]-N-[[4-(hydroxymethyl)-2-(2-hydroxypropan-2-yl)-1,3-thiazol-5-yl]sulfonyl]acetamide: (DMSO, ppm): δ 7.04 (s, 1H), 5.03 (s, 2H), 4.87 (s, 2H), 4.71 (s, 2H), 1.47 (s, 6H), 1.04 (d, J=6.7 Hz, 6H), 0.99 (d, J=7.1 Hz, 6H).

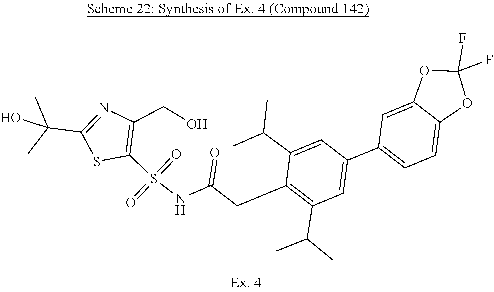 [SMILES: CC(C)c1cc(-c2ccc3c(c2)OC(F)(F)O3)cc(C(C)C)c1CC(=O)NS(=O)(=O)c1sc(C(C)(C)O)nc1CO]

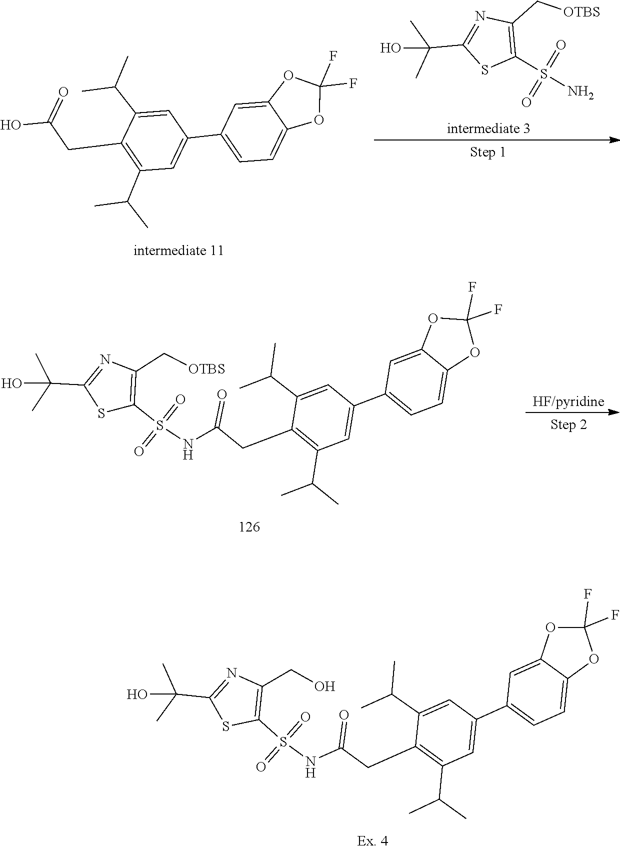 [SMILES: CC(C)c1cc(-c2ccc3c(c2)OC(F)(F)O3)cc(C(C)C)c1CC(=O)O ; CC(C)(O)c1nc(CO[Si](C)(C)C(C)(C)C)c(S(N)(=O)=O)s1 ; CC(C)c1cc(-c2ccc3c(c2)OC(F)(F)O3)cc(C(C)C)c1CC(=O)NS(=O)(=O)c1sc(C(C)(C)O)nc1CO[Si](C)(C)C(C)(C)C ; CC(C)c1cc(-c2ccc3c(c2)OC(F)(F)O3)cc(C(C)C)c1CC(=O)NS(=O)(=O)c1sc(C(C)(C)O)nc1CO]

### 1. Synthesis of N-[(4-[[(tert-butyldimethylsilyl)oxy]methyl]-2-(2-hydroxypropan-2-yl)-1,3-thiazol-5-yl)sulfonyl]-2-[4-(2,2-difluoro-2H-1,3-benzodioxol-5-yl)-2,6-bis(propan-2-yl)phenyl]acetamide

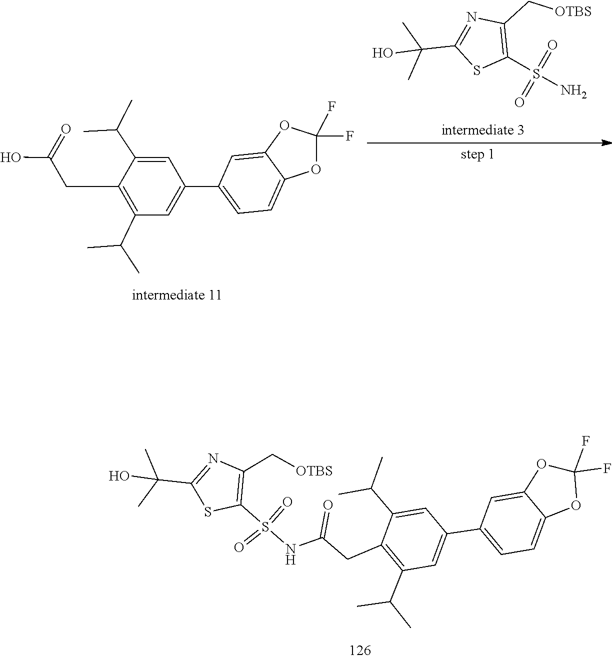 [SMILES: CC(C)c1cc(-c2ccc3c(c2)OC(F)(F)O3)cc(C(C)C)c1CC(=O)O ; CC(C)(O)c1nc(CO[Si](C)(C)C(C)(C)C)c(S(N)(=O)=O)s1 ; CC(C)c1cc(-c2ccc3c(c2)OC(F)(F)O3)cc(C(C)C)c1CC(=O)NS(=O)(=O)c1sc(C(C)(C)O)nc1CO[Si](C)(C)C(C)(C)C]

Into a 50-mL round-bottom flask, was placed 2-[4-(2,2-difluoro-2H-1,3-benzodioxol-5-yl)-2,6-bis(propan-2-yl)phenyl]acetic acid (100 mg), DCM (5 mL), and oxalyl chloride (0.5 mL). This was followed by the addition of N,N-dimethylformamide (0.05 mL) dropwise with stirring. The resulting solution was stirred for 30 min at room temperature. The resulting mixture was concentrated under vacuum.

Into a 50-mL round-bottom flask, was placed 2-[4-(2,2-difluoro-2H-1,3-benzodioxol-5-yl)-2,6-bis(propan-2-yl)phenyl]acetyl chloride (100 mg) and THE (15 mL). This was followed by the addition of NaH (21.84 mg, 60%). To this was added 4-[[(tert-butyldimethylsilyl)oxy]methyl]-2-(2-hydroxypropan-2-yl)-1,3-thiazole-5-sulfonamide (100 mg). The resulting solution was stirred for 1 hr at room temperature. The reaction was then quenched by the addition of 5 mL of water. The resulting solution was extracted with 3×5 ml of ethyl acetate; the organic layers combined and dried over anhydrous sodium sulfate. The solids were filtered out. The filtrate was concentrated under vacuum. This resulted in 100 mg of N-[(4-[[(tert-butyldimethylsilyl)oxy]methyl]-2-(2-hydroxypropan-2-yl)-1,3-thiazol-5-yl)sulfonyl]-2-[4-(2,2-difluoro-2H-1,3-benzodioxol-5-yl)-2,6-bis(propan-2-yl)phenyl]acetamide as a yellow solid.

### 2. Synthesis of 2-[4-(2,2-difluoro-2H-1,3-benzodioxol-5-yl)-2,6-bis(propan-2-yl)phenyl]-N-[[4-(hydroxymethyl)-2-(2-hydroxypropan-2-yl)-1,3-thiazol-5-yl]sulfonyl]acetamide

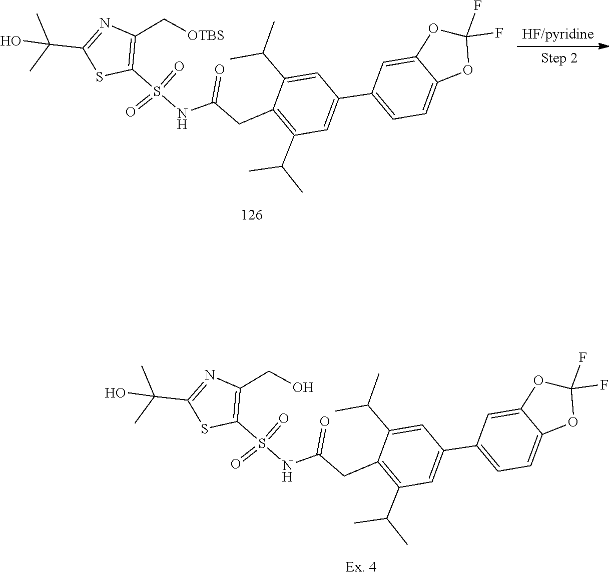 [SMILES: CC(C)c1cc(-c2ccc3c(c2)OC(F)(F)O3)cc(C(C)C)c1CC(=O)NS(=O)(=O)c1sc(C(C)(C)O)nc1CO[Si](C)(C)C(C)(C)C ; CC(C)c1cc(-c2ccc3c(c2)OC(F)(F)O3)cc(C(C)C)c1CC(=O)NS(=O)(=O)c1sc(C(C)(C)O)nc1CO]

Into a 50-mL round-bottom flask, was placed N-[(4-[[(tert-butyldimethylsilyl)oxy]methyl]-2-(2-hydroxypropan-2-yl)-1,3-thiazol-5-yl)sulfonyl]-2-[4-(2,2-difluoro-2H-1,3-benzodioxol-5-yl)-2,6-bis(propan-2-yl)phenyl]acetamide (100 mg), THE (5 mL), and HF Pyridine (0.5 mL). The resulting solution was stirred for 10 hr at room temperature. The resulting mixture was concentrated under vacuum. The crude product was purified by Prep-TLC (diluted with PE/EtOAc=1:1). This resulted in 15.3 mg of 2-[4-(2,2-difluoro-2H-1,3-benzodioxol-5-yl)-2,6-bis(propan-2-yl)phenyl]-N-[[4-(hydroxymethyl)-2-(2-hydroxypropan-2-yl)-1,3-thiazol-5-yl]sulfonyl]acetamide as a white solid.

LC-MS-2-[4-(2,2-difluoro-2H-1,3-benzodioxol-5-yl)-2,6-bis(propan-2-yl)phenyl]-N-[[4-(hydroxymethyl)-2-(2-hydroxypropan-2-yl)-1,3-thiazol-5-yl]sulfonyl]acetamide: (ES, m/z): 610.16, retention time: 1.898. Method: CORTECS C18+, 50*2.1 mm, 0.5 uL injection, 0.8 mL/min flowrate, 90-900 amu scan range, 254 nm UV detection. Mobile phase A: Water (0.1% FA) and Mobile Phase B: MeCN. 10% MPB to 95.0% in 2.0 min, hold at 95% MPB for 0.6 min, 95% MPB to 10% in 0.2 min, then equilibration to 10% MPB for 0.2 min.

H-NMR-2-[4-(2,2-difluoro-2H-1,3-benzodioxol-5-yl)-2,6-bis(propan-2-yl)phenyl]-N-[[4-(hydroxymethyl)-2-(2-hydroxypropan-2-yl)-1,3-thiazol-5-yl]sulfonyl]acetamide: (300 MHz, Methanol-d4) δ 7.43-7.30 (m, 2H), 7.30-7.20 (m, 3H), 3.81 (s, 2H), 3.08 (m, J=6.9 Hz, 2H), 1.59 (s, 6H), 1.19 (d, J=6.8 Hz, 12H).

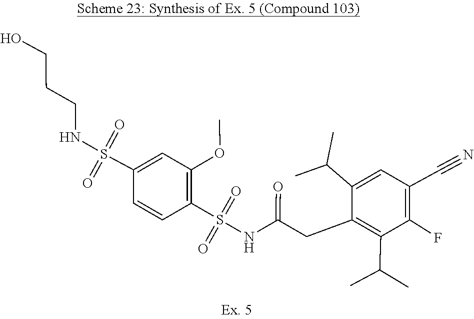 [SMILES: COc1cc(S(=O)(=O)NCCCO)ccc1S(=O)(=O)NC(=O)Cc1c(C(C)C)cc(C#N)c(F)c1C(C)C]

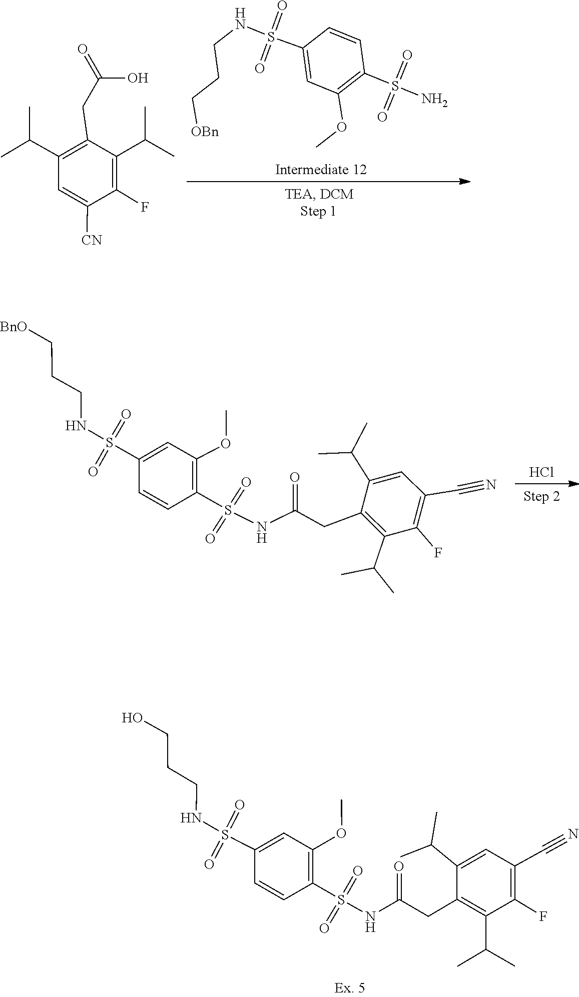

### 1. Synthesis of N-(4-[[3-(benzyloxy)propyl]sulfamoyl]-2-methoxybenzenesulfonyl)-2-[4-cyano-3-fluoro-2,6-bis(propan-2-yl)phenyl]acetamide

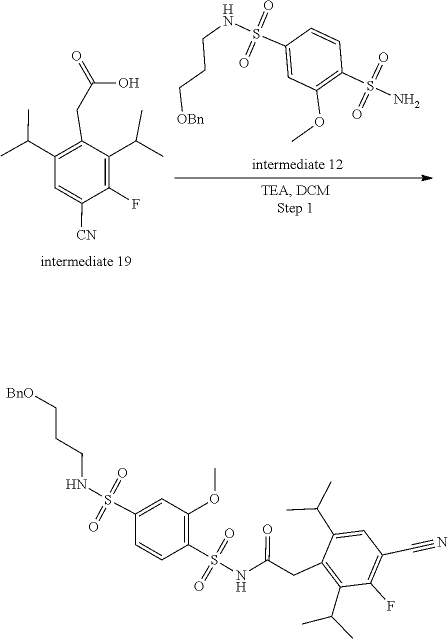

Into a 100-mL round-bottom flask, was placed N1-[3-(benzyloxy)propyl]-3-methoxybenzene-1,4-disulfonamide (500 mg, 1.2 mmol, 1 equiv), DCM (50 m), 2-[4-cyano-3-fluoro-2,6-bis(propan-2-yl)phenyl]acetyl chloride (408 mg, 1.4 mmol, 1.20 equiv), and TEA (488 mg, 4.8 mmol, 4 equiv). The resulting solution was stirred for 1 day at room temperature, after which it was concentrated. The crude product was purified by Prep-HPLC with the following conditions: Column, XBridge Prep OBD C18 Column 30*150 mm 5 um; mobile phase, A:Water(10 MMOL/L NH₄HCO₃+0.1% NH₃.H₂O), B: ACN; Flow rate: 40 mL/min; Gradient: 36% B to 60% B in 6; Detector, 254/210 nm. This resulted in 200 mg (25.13%) of N-(4-[[3-(benzyloxy)propyl]sulfamoyl]-2-methoxybenzenesulfonyl)-2-[4-cyano-3-fluoro-2,6-bis(propan-2-yl)phenyl]acetamide as a white solid.

LC-MS-N-(4-[[3-(benzyloxy)propyl]sulfamoyl]-2-methoxybenzenesulfonyl)-2-[4-cyano-3-fluoro-2,6-bis(propan-2-yl)phenyl]acetamide(Method N): (ES, m/z): [M+H]⁺=660, retention time: 1.412.

H-NMR—N-(4-[[3-(benzyloxy)propyl]sulfamoyl]-2-methoxybenzenesulfonyl)-2-[4-cyano-3-fluoro-2,6-bis(propan-2-yl)phenyl]acetamide: 1H NMR (300 MHz, Methanol-d4) δ: 7.48 (s, 2H), 7.32 (s, 2H), 4.45 (s, 1H), 4.04 (s, 2H), 3.85 (s, 1H), 3.48 (d, J=3.0 Hz, 1H), 2.97 (s, 2H), 1.75 (s, 1H), 1.31 (s, 2H), 1.20-1.18 (m, 3H), 1.18-1.14 (m, 4H), 1.02-0.94 (m, 1H).

### 2. Synthesis of 2-[4-cyano-3-fluoro-2,6-bis(propan-2-yl)phenyl]-N-[4-[(3-hydroxypropyl)sulfamoyl]-2-methoxybenzenesulfonyl]acetamide

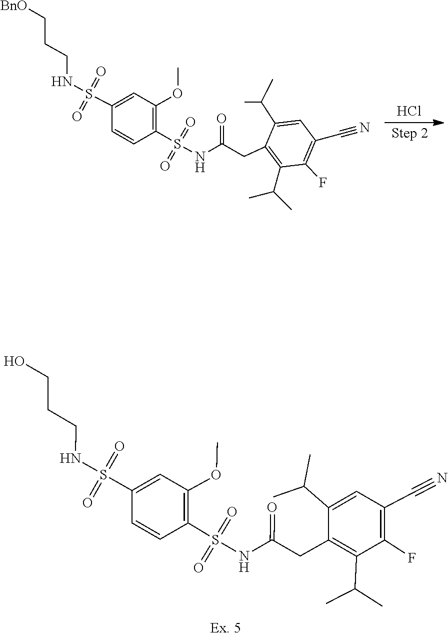 [SMILES: COc1cc(S(=O)(=O)NCCCOCc2ccccc2)ccc1S(=O)(=O)NC(=O)Cc1c(C(C)C)cc(C#N)c(F)c1C(C)C ; Cl ; COc1cc(S(=O)(=O)NCCCO)ccc1S(=O)(=O)NC(=O)Cc1c(C(C)C)cc(C#N)c(F)c1C(C)C]

Into a 100-mL round-bottom flask, was placed N-(4-[[3-(benzyloxy)propyl]sulfamoyl]-2-methoxybenzenesulfonyl)-2-[4-cyano-3-fluoro-2,6-bis(propan-2-yl)phenyl]acetamide (60 mg), dioxane (2 mL), and HCl (2 mL). The resulting solution was stirred for 1 day at 40 degrees C. The resulting mixture was concentrated. The crude product (40 mg) was purified by Prep-HPLC with the following conditions: Column, XBridge Prep OBD C18 Column 30*150 mm 5 um; mobile phase, A:Water(10 MMOL/L NH4HCO3+0.1$ NH3.H2O), B: ACN; Flow rate: 40 mL/min; Gradient: 36% B to 60% B in 6 min; Detector, 254/210 nm. This resulted in 20 mg (36.1%) of 2-[4-cyano-3-fluoro-2,6-bis(propan-2-yl)phenyl]-N-[4-[(3-hydroxypropyl)sulfamoyl]-2-methoxybenzenesulfonyl]acetamide as a white solid.

LC-MS-2-[4-cyano-3-fluoro-2,6-bis(propan-2-yl)phenyl]-N-[4-[(3-hydroxypropyl)sulfamoyl]-2-methoxybenzenesulfonyl]acetamide(Method N): (ES, m/z): [M+H]⁺=570

H-NMR-2-[4-cyano-3-fluoro-2,6-bis(propan-2-yl)phenyl]-N-[4-[(3-hydroxypropyl)sulfamoyl]-2-methoxybenzenesulfonyl]acetamide: ¹H NMR (300 MHz, Methanol-d4) δ: 8.11 (d, J=8.2 Hz, 1H), 7.61 (s, 1H), 7.53 (d, J=8.4 Hz, 1H), 7.45 (d, J=6.3 Hz, 1H), 4.11 (s, 3H), 3.89 (s, 2H), 3.57 (t, J=6.1 Hz, 2H), 2.97 (t, J=6.0 Hz, 4H), 1.73-1.64 (m, 2H), 1.24-1.09 (m, 12H).

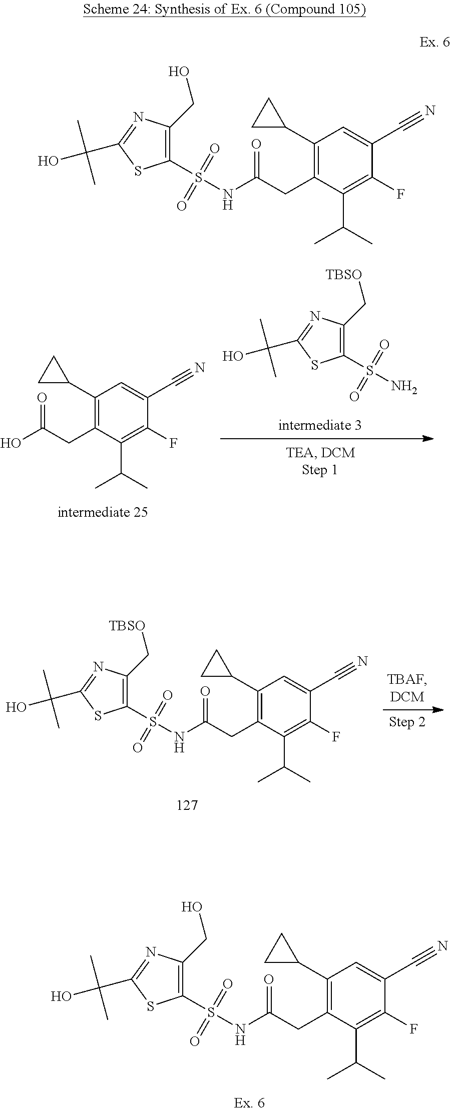

### 1. Synthesis of N-[(4-[[(tert-butyldimethylsilyl)oxy]methyl]-2-(2-hydroxypropan-2-yl)-1,3-thiazol-5-yl)sulfonyl]-2-[4-cyano-6-cyclopropyl-3-fluoro-2-(propan-2-yl)phenyl]acetamide

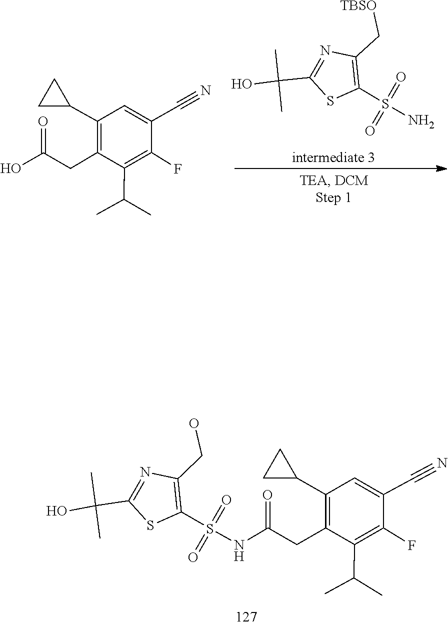

Into a 25-mL round-bottom flask purged with and maintained under an inert atmosphere of nitrogen, was placed 2-[4-cyano-6-cyclopropyl-3-fluoro-2-(propan-2-yl)phenyl]acetic acid (100 mg, 0.383 mmol, 1 equiv), DCM (5 mL, 0.059 mmol, 0.15 equiv), (COCl)₂ (145.73 mg, 1.148 mmol, 3 equiv), and DMF (0.1 mL). The resulting solution was stirred for 1 hr at room temperature in a water bath. The resulting mixture was concentrated. This resulted in 100 mg (93.41%) of 2-[4-cyano-6-cyclopropyl-3-fluoro-2-(propan-2-yl)phenyl]acetyl chloride as a yellow solid.

Into a 50-mL 3-necked round-bottom flask purged with and maintained under an inert atmosphere of nitrogen, was placed 4-[[(tert-butyldimethylsilyl)oxy]methyl]-2-(2-hydroxypropan-2-yl)-1,3-thiazole-5-sulfonamide (131.04 mg, 0.357 mmol, 1 equiv), DCM (5 mL, 0.059 mmol, 0.16 equiv), TEA (108.52 mg, 1.072 mmol, 3 equiv), and 2-[4-cyano-6-cyclopropyl-3-fluoro-2-(propan-2-yl)phenyl]acetyl chloride (100 mg, 0.357 mmol, 1 equiv). The resulting solution was stirred for 1 hr at 0 degrees C. in a water/ice bath. The reaction was then quenched by the addition of 0.1 mL of water. The resulting mixture was concentrated. This resulted in 150 mg (68.81%) of N-[(4-[[(tert-butyldimethylsilyl)oxy]methyl]-2-(2-hydroxypropan-2-yl)-1,3-thiazol-5-yl)sulfonyl]-2-[4-cyano-6-cyclopropyl-3-fluoro-2-(propan-2-yl)phenyl]acetamide as a yellow solid.

LC-MS-N-[(4-[[(tert-butyldimethylsilyl)oxy]methyl]-2-(2-hydroxypropan-2-yl)-1,3-thiazol-5-yl)sulfonyl]-2-[4-cyano-6-cyclopropyl-3-fluoro-2-(propan-2-yl)phenyl]acetamide: (ES, m/z): [M+H]⁺=610.4, retention time: 1.107. Method: Omega, 30*2.1 mm, 3.0 um column, 0.7 uL injection, 1.2 mL/min flow rate, 90-900 amu scan range, 190-400 nm UV range, Mobile phase A: Water (0.09% FA) and Mobile Phase B: MeCN(0.1% FA). 5% MPB to 95% in 0.9 min, hold at 95% MPB for 0.5 min, 95% MPB to 5% in 0.03 min.

### 2. Synthesis of 2-[4-cyano-6-cyclopropyl-3-fluoro-2-(propan-2-yl)phenyl]-N-[[4-(hydroxymethyl)-2-(2-hydroxypropan-2-yl)-1,3-thiazol-5-yl]sulfonyl]acetamide

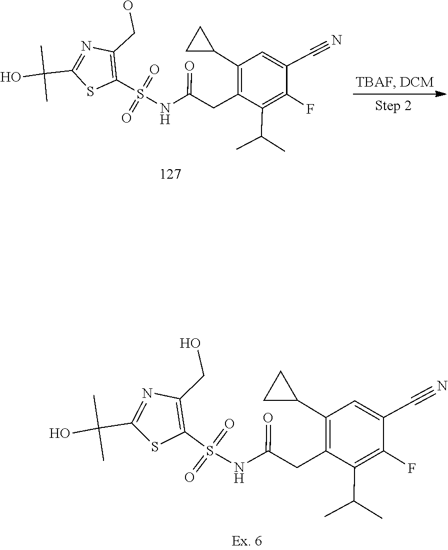

Into a 50-mL round-bottom flask purged and maintained with an inert atmosphere of nitrogen, was placed N-[(4-[[(tert-butyldimethylsilyl)oxy]methyl]-2-(2-hydroxypropan-2-yl)-1,3-thiazol-5-yl)sulfonyl]-2-[4-cyano-6-cyclopropyl-3-fluoro-2-(propan-2-yl)phenyl]acetamide (100 mg, 0.164 mmol, 1 equiv), THE (10 mL, 0.139 mmol, 0.85 equiv), TBAF (01.0 mL, 1.0 M THF). The resulting solution was stirred for 1 hr at room temperature in a water/ice bath. The resulting mixture was concentrated. The solids were filtered out. The crude product (100 mg) was purified by Prep-HPLC with the following conditions (Prep-HPLC-008): Column, XBridge Shield RP18 OBD Column, 19*250 mm, 10 um; mobile phase, water (10 MMOL/L NH4HCO3) and ACN (15% Phase B up to 47% in 7 min); Detector, UV. 30 mg product was obtained. This resulted in 30 mg (36.92%) of 2-[4-cyano-6-cyclopropyl-3-fluoro-2-(propan-2-yl)phenyl]-N-[[4-(hydroxymethyl)-2-(2-hydroxypropan-2-yl)-1,3-thiazol-5-yl]sulfonyl]acetamide as a white solid.

LC-MS-2-[4-cyano-6-cyclopropyl-3-fluoro-2-(propan-2-yl)phenyl]-N-[[4-(hydroxymethyl)-2-(2-hydroxypropan-2-yl)-1,3-thiazol-5-yl]sulfonyl]acetamide (Method J): (ES, m/z): [M+H]⁺=496.1, retention time: 0.855.

¹HNMR-2-[4-cyano-6-cyclopropyl-3-fluoro-2-(propan-2-yl)phenyl]-N-[[4-(hydroxymethyl)-2-(2-hydroxypropan-2-yl)-1,3-thiazol-5-yl]sulfonyl]acetamide (400 MHz, DMSO-d6) δ 7.32 (d, J=6.3 Hz, 1H), 7.07 (t, J=44.1 Hz, 3H), 5.99 (s, 1H), 5.02 (s, 1H), 4.61 (s, 2H), 3.78 (s, 2H), 3.19-3.11 (m, 1H), 1.88 (s, 1H), 1.45 (s, 6H), 1.13 (dd, J=7.0, 1.6 Hz, 6H), 0.87-0.78 (m, 2H), 0.58 (q, J=5.3 Hz, 2H).

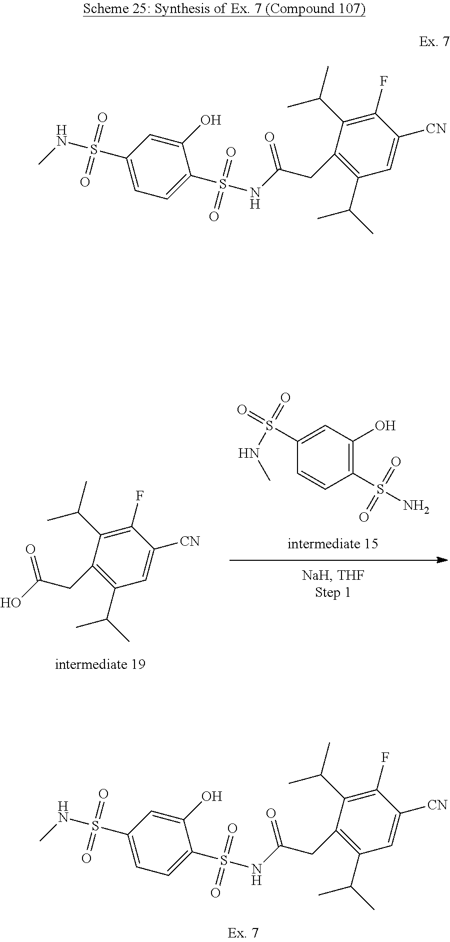 [SMILES: CNS(=O)(=O)c1ccc(S(=O)(=O)NC(=O)Cc2c(C(C)C)cc(C#N)c(F)c2C(C)C)c(O)c1 ; CC(C)c1cc(C#N)c(F)c(C(C)C)c1CC(=O)O ; CNS(=O)(=O)c1ccc(S(N)(=O)=O)c(O)c1 ; CNS(=O)(=O)c1ccc(S(=O)(=O)NC(=O)Cc2c(C(C)C)cc(C#N)c(F)c2C(C)C)c(O)c1]

### 1. Synthesis of 2-[4-cyano-3-fluoro-2,6-bis(propan-2-yl)phenyl]-N-[2-hydroxy-4-(methylsulfamoyl)benzenesulfonyl]acetamide

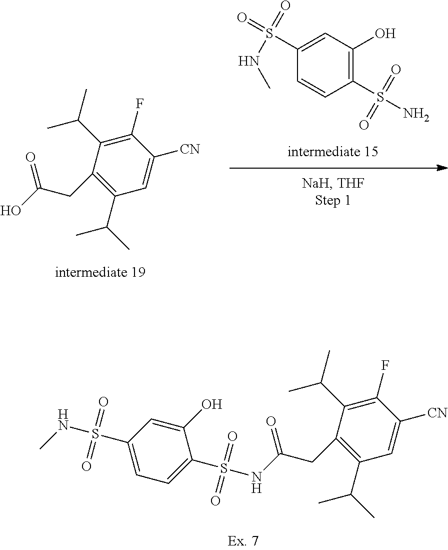 [SMILES: CC(C)c1cc(C#N)c(F)c(C(C)C)c1CC(=O)O ; CNS(=O)(=O)c1ccc(S(N)(=O)=O)c(O)c1 ; CNS(=O)(=O)c1ccc(S(=O)(=O)NC(=O)Cc2c(C(C)C)cc(C#N)c(F)c2C(C)C)c(O)c1]

Into a 50 mL round-bottom flask were added 2-[4-cyano-3-fluoro-2,6-bis(propan-2-yl)phenyl]acetic acid (200 mg, 0.76 mmol, 1 equiv) and DCM (10 mL) at room temperature. To a stirred solution of 2-[4-cyano-3-fluoro-2,6-bis(propan-2-yl)phenyl]acetic acid (200 mg, 0.76 mmol, 1 equiv) and DMF(0.02 mL, 0.26 mmol, 0.34 equiv) in DCM(0.5 mL) was added oxalyl chloride (0.4 mL) dropwise at room temperature. The resulting mixture was concentrated under reduced pressure to afford 2-[4-cyano-3-fluoro-2,6-bis(propan-2-yl)phenyl]acetyl chloride.

Into a 50-mL round-bottom flask, was placed a solution of 3-hydroxy-N1-methylbenzene-1,4-disulfonamide (370 mg, 1.39 mmol, 1 equiv) in THE (10 mL), NaH (120 mg, 5.00 mmol, 3.60 equiv), 2-[4-cyano-3-fluoro-2,6-bis(propan-2-yl)phenyl]acetyl chloride (500 mg, 1.77 mmol, 1.28 equiv) were added. The resulting solution was stirred for 1 hr at room temperature. The reaction was then quenched by the addition of 50 mL of water. The resulting solution was extracted with 3×50 ml of ethyl acetate and dried over anhydrous sodium sulfate. The solids were filtered out. The filtrate was concentrated under vacuum. The crude product was purified by Prep-HPLC with the following conditions (2#SHIMADZU (HPLC-01)): Column, XBridge Prep C18 OBD Column, 5 um, 19*150 mm; mobile phase, water (10 mmol/L NH4HCO3) and ACN (12% Phase B up to 50% in 10 min); Detector, UV254/210 nm. This resulted in 292.5 mg (41.15%) of 2-[4-cyano-3-fluoro-2,6-bis(propan-2-yl)phenyl]-N-[2-hydroxy-4-(methylsulfamoyl)benzenesulfonyl]acetamide as a light yellow solid.

LC-MS-2-[4-cyano-3-fluoro-2,6-bis(propan-2-yl)phenyl]-N-[2-hydroxy-4-(methylsulfamoyl)benzenesulfonyl]acetamide(Method M): (ES, m/z): [M+1]⁺=512.2, retention time: 2.363.

H-NMR-2-[4-cyano-3-fluoro-2,6-bis(propan-2-yl)phenyl]-N-[2-hydroxy-4-(methylsulfamoyl)benzenesulfonyl]acetamide (300 MHz, DMSO-d₆) δ: 7.88 (d, J=8.10 Hz, 1H), 7.41 (d, J=6.30 Hz, 1H), 7.31 (d, J=1.80 Hz, 1H), 7.28 (s, 1H), 3.78 (s, 2H), 3.32-3.01 (m, 2H), 2.53 (s, 3H), 1.22-1.10 (m, 12H).

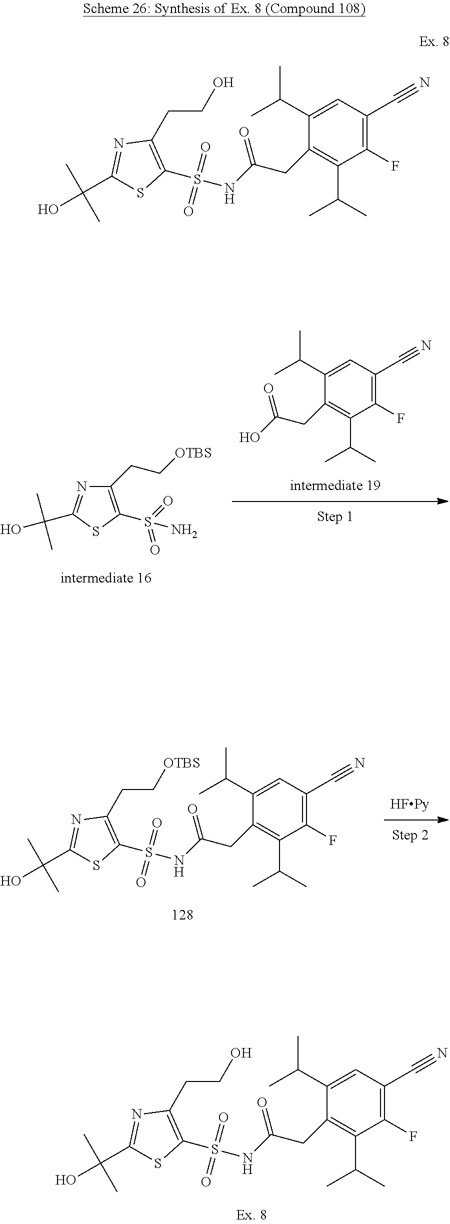 [SMILES: CC(C)c1cc(C#N)c(F)c(C(C)C)c1CC(=O)NS(=O)(=O)c1sc(C(C)(C)O)nc1CCO ; CC(C)(O)c1nc(CCO[Si](C)(C)C(C)(C)C)c(S(N)(=O)=O)s1 ; CC(C)c1cc(C#N)c(F)c(C(C)C)c1CC(=O)O ; CC(C)c1cc(C#N)c(F)c(C(C)C)c1CC(=O)NS(=O)(=O)c1sc(C(C)(C)O)nc1CCO[Si](C)(C)C(C)(C)C ; CC(C)c1cc(C#N)c(F)c(C(C)C)c1CC(=O)NS(=O)(=O)c1sc(C(C)(C)O)nc1CCO]

### 1. Synthesis of N-((4-(2-((tert-butyldimethylsilyl)oxy)ethyl)-2-(2-hydroxypropan-2-yl)thiazol-5-yl)sulfonyl)-2-(4-cyano-3-fluoro-2,6-diisopropylphenyl)acetamide

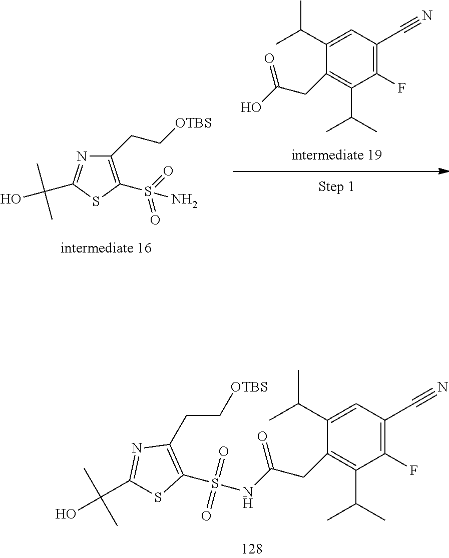 [SMILES: CC(C)(O)c1nc(CCO[Si](C)(C)C(C)(C)C)c(S(N)(=O)=O)s1 ; CC(C)c1cc(C#N)c(F)c(C(C)C)c1CC(=O)O ; CC(C)c1cc(C#N)c(F)c(C(C)C)c1CC(=O)NS(=O)(=O)c1sc(C(C)(C)O)nc1CCO[Si](C)(C)C(C)(C)C]

To a stirred solution/mixture of 2-[4-cyano-3-fluoro-2,6-bis(propan-2-yl)phenyl]acetic acid (100 mg, 0.38 mmol, 1 equiv) and DMF (0.04 mL, 0.52 mmol, 1.36 equiv) in DCM (5 mL) was added (COCl)₂ (144.6 mg, 1.14 mmol, 3.00 equiv) dropwise/in portions at room temperature. The resulting mixture was stirred for 30 min at room temperature. The resulting mixture was concentrated under vacuum to afford 2-[4-cyano-3-fluoro-2,6-bis(propan-2-yl)phenyl]acetyl chloride.

A solution/mixture of 4-[2-[(tert-butyldimethylsilyl)oxy]ethyl]-2-(2-hydroxypropan-2-yl)-1,3-thiazole-5-sulfonamide(100 mg, 0.26 mmol, 1 equiv), TEA (79.8 mg, 0.79 mmol, 3 equiv) and 2-[4-cyano-3-fluoro-2,6-bis(propan-2-yl)phenyl]acetyl chloride (88.8 mg, 0.32 mmol, 1.20 equiv) in DCM(5 mL) was stirred for 2 h at room temperature. The resulting mixture was concentrated under vacuum to provide the titled compound.

LC-MS-N-((4-(2-((tert-butyldimethylsilyl)oxy)ethyl)-2-(2-hydroxypropan-2-yl)thiazol-5-yl)sulfonyl)-2-(4-cyano-3-fluoro-2,6-diisopropylphenyl)acetamide (Method G): (ES, m/z): [M+H]⁺=626.3, retention time: 1.191.

### 2. Synthesis of 2-[4-cyano-3-fluoro-2,6-bis(propan-2-yl)phenyl]-N-[[4-(2-hydroxyethyl)-2-(2-hydroxypropan-2-yl)-1,3-thiazol-5-yl]sulfonyl]acetamide

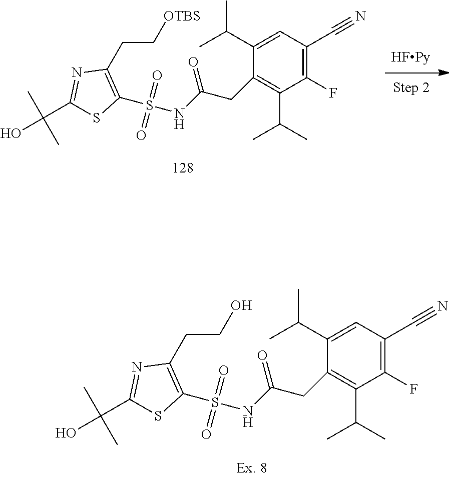 [SMILES: CC(C)c1cc(C#N)c(F)c(C(C)C)c1CC(=O)NS(=O)(=O)c1sc(C(C)(C)O)nc1CCO[Si](C)(C)C(C)(C)C ; CC(C)c1cc(C#N)c(F)c(C(C)C)c1CC(=O)NS(=O)(=O)c1sc(C(C)(C)O)nc1CCO]

Into a 50-mL round-bottom flask, was placed N-[(4-[2-[(tert-butyldimethylsilyl)oxy]ethyl]-2-(2-hydroxypropan-2-yl)-1,3-thiazol-5-yl)sulfonyl]-2-[4-cyano-3-fluoro-2,6-bis(propan-2-yl)phenyl]acetamide (140 mg, 0.22 mmol, 1 equiv), THE (5 mL), and HF/Py (223.7 mg, 2.24 mmol, 10.00 equiv). The resulting solution was stirred for 1 h at room temperature, after which it was concentrated. The crude product was purified by Prep-HPLC with the following conditions (Prep-HPLC-018): Column, XBridge Shield RP18 OBD Column, 19*250 mm, 10 um; mobile phase, Water(10 mmol/L NH₄HCO₃) and ACN (22% PhaseB up to 45% in 9 min); Detector, UV 254/210 nm. This resulted in 27.6 mg (24.12%) of 2-[4-cyano-3-fluoro-2,6-bis(propan-2-yl)phenyl]-N-[[4-(2-hydroxyethyl)-2-(2-hydroxypropan-2-yl)-1,3-thiazol-5-yl]sulfonyl]acetamide as a yellow solid.

LC-MS-2-[4-cyano-3-fluoro-2,6-bis(propan-2-yl)phenyl]-N-[[4-(2-hydroxyethyl)-2-(2-hydroxypropan-2-yl)-1,3-thiazol-5-yl]sulfonyl]acetamide(Method M): (ES, m/z): [M+H]⁺=512.3, retention time: 1.270.

H-NMR-2-[4-cyano-3-fluoro-2,6-bis(propan-2-yl)phenyl]-N-[[4-(2-hydroxyethyl)-2-(2-hydroxypropan-2-yl)-1,3-thiazol-5-yl]sulfonyl]acetamide: ¹H NMR (400 MHz, CD₃OD-d₄) δ 7.47 (d, J=6.3 Hz, 1H), 3.96-3.85 (m, 4H), 3.30-3.25 (m, 2H), 3.03-3.00 (t, J=5.6 Hz, 2H), 1.57 (s, 6H), 1.21-1.94 (d, J=6.8 Hz, 6H), 1.15-1.13 (d, J=6.8 Hz, 6H).

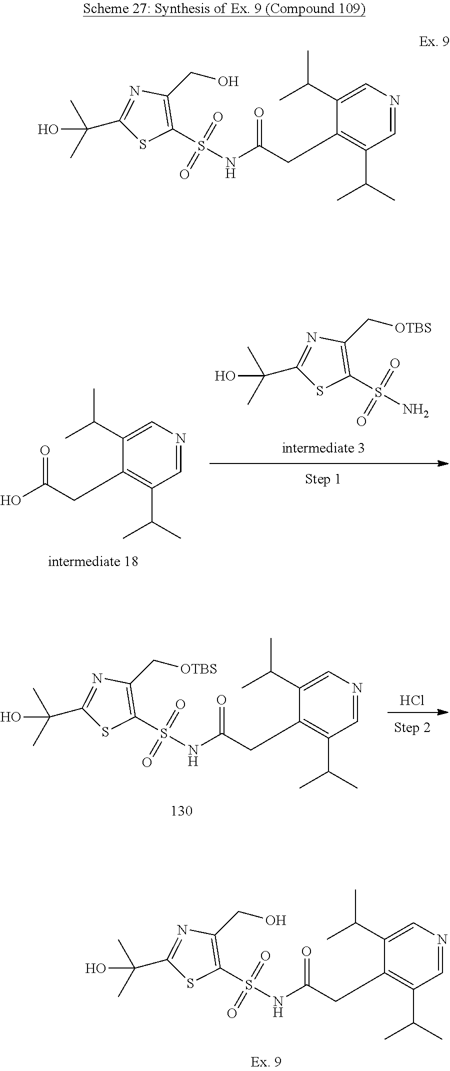 [SMILES: CC(C)c1cncc(C(C)C)c1CC(=O)NS(=O)(=O)c1sc(C(C)(C)O)nc1CO ; CC(C)c1cncc(C(C)C)c1CC(=O)O ; CC(C)(O)c1nc(CO[Si](C)(C)C(C)(C)C)c(S(N)(=O)=O)s1 ; CC(C)c1cncc(C(C)C)c1CC(=O)NS(=O)(=O)c1sc(C(C)(C)O)nc1CO[Si](C)(C)C(C)(C)C ; Cl ; CC(C)c1cncc(C(C)C)c1CC(=O)NS(=O)(=O)c1sc(C(C)(C)O)nc1CO]

### 1. Synthesis of N-(((4-(((tert-butyldimethylsilyl)oxy)methyl)-5-(2-hydroxypropan-2-yl)thiazol-2-yl)methyl)sulfonyl)-2-(3,5-diisopropylpyridin-4-yl)acetamide

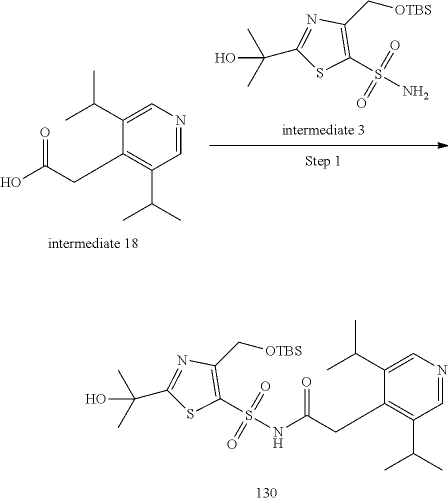 [SMILES: CC(C)c1cncc(C(C)C)c1CC(=O)O ; CC(C)(O)c1nc(CO[Si](C)(C)C(C)(C)C)c(S(N)(=O)=O)s1 ; CC(C)c1cncc(C(C)C)c1CC(=O)NS(=O)(=O)c1sc(C(C)(C)O)nc1CO[Si](C)(C)C(C)(C)C]

Into a 50-mL round-bottom flask purged with and maintained under an inert atmosphere of nitrogen, was placed a solution of 2-(3,5-diisopropylpyridin-4-yl)acetic acid (180 mg, 0.81 mmol, 1.00 equiv.) and (4-(((tert-butyldimethylsilyl)oxy)methyl)-5-(2-hydroxypropan-2-yl)thiazol-2-yl)methanesulfonamide (310 mg, 0.81 mmol, 1.00 equiv.) in DCM (20 mL). To the solution was added HATU (616 mg, 1.62 mmol, 2.00 equiv.) and DIEA (313 mg, 2.43 mmol, 3.00 equiv.). The resulting solution was stirred for 2 h at room temperature. The reaction was then quenched by the addition of 20 mL of water and diluted with 50 mL of DCM. The resulting mixture was washed with 1×100 mL of water and 1×100 mL of brine. The combined organic phase was dried over anhydrous magnesium sulfate and concentrated under vacuum. The residue thus obtained was applied onto a silica gel column which was eluted with ethyl acetate/petroleum ether (1:10). This resulted in 70 mg (15%) of N-(((4-(((tert-butyldimethylsilyl)oxy)methyl)-5-(2-hydroxypropan-2-yl)thiazol-2-yl)methyl)sulfonyl)-2-(3,5-diisopropylpyridin-4-yl)acetamide as a colorless oil.

LC-MS—N-(((4-(((tert-butyldimethylsilyl)oxy)methyl)-5-(2-hydroxypropan-2-yl)thiazol-2-yl)methyl)sulfonyl)-2-(3,5-diisopropylpyridin-4-yl)acetamide: (ES, m/z): [M+H]⁺: 569.24.

### 2. Synthesis of 2-(3,5-diisopropylpyridin-4-yl)-N-((4-(hydroxymethyl)-2-(2-hydroxypropan-2-yl)thiazol-5-yl)sulfonyl)acetamide

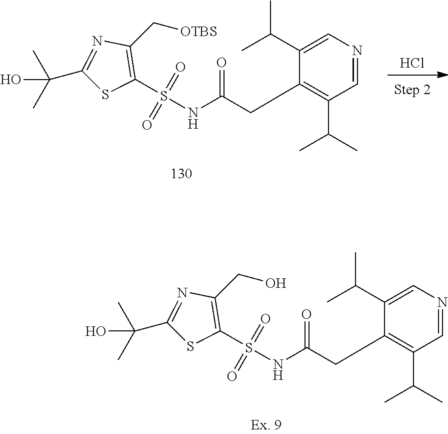 [SMILES: CC(C)c1cncc(C(C)C)c1CC(=O)NS(=O)(=O)c1sc(C(C)(C)O)nc1CO[Si](C)(C)C(C)(C)C ; Cl ; CC(C)c1cncc(C(C)C)c1CC(=O)NS(=O)(=O)c1sc(C(C)(C)O)nc1CO]

Into a 25-mL sealed tube purged and maintained with an inert atmosphere of nitrogen, was placed a solution of N-(((4-(((tert-butyldimethylsilyl)oxy)methyl)-5-(2-hydroxypropan-2-yl)thiazol-2-yl)methyl)sulfonyl)-2-(3,5-diisopropylpyridin-4-yl)acetamide (70 mg, 0.12 mmol, 1.00 equiv.) in DCM (5 mL). To the solution were added HCl/dioxane solution (2 mL). The resulting solution was stirred for 3 h at room temperature. The reaction was then concentrated to afford the crude product. Then 22 mg of 2-(3,5-diisopropylpyridin-4-yl)-N-((4-(hydroxymethyl)-2-(2-hydroxypropan-2-yl)thiazol-5-yl)sulfonyl)acetamide was purified by prep-HPLC with the following conditions (PREP_HPLC_MC4): Column: XBridge Shield RP18 OBD Column 19*250 mm, 10 um; Mobile Phase A:Water (10 MMOL/L NH4HCO3), Mobile Phase B: ACN; Flow rate: 25 mL/min; Gradient: 10% B to 30% B in 10 min; 254/210 nm; Rt: 7.33 min.

LC-MS-2-(3,5-diisopropylpyridin-4-yl)-N-((4-(hydroxymethyl)-2-(2-hydroxypropan-2-yl)thiazol-5-yl)sulfonyl)acetamide(Method M): (ES, m/z): [M+H]⁺: 455.15.

¹H NMR-2-(3,5-diisopropylpyridin-4-yl)-N-((4-(hydroxymethyl)-2-(2-hydroxypropan-2-yl)thiazol-5-yl)sulfonyl)acetamide: (400 MHz, Methanol-d4) δ 8.33 (s, 2H), 4.86 (s, 2H), 3.85 (s, 2H), 3.19 (p, J=6.9 Hz, 2H), 1.56 (s, 6H), 1.22 (d, J=6.8 Hz, 12H).

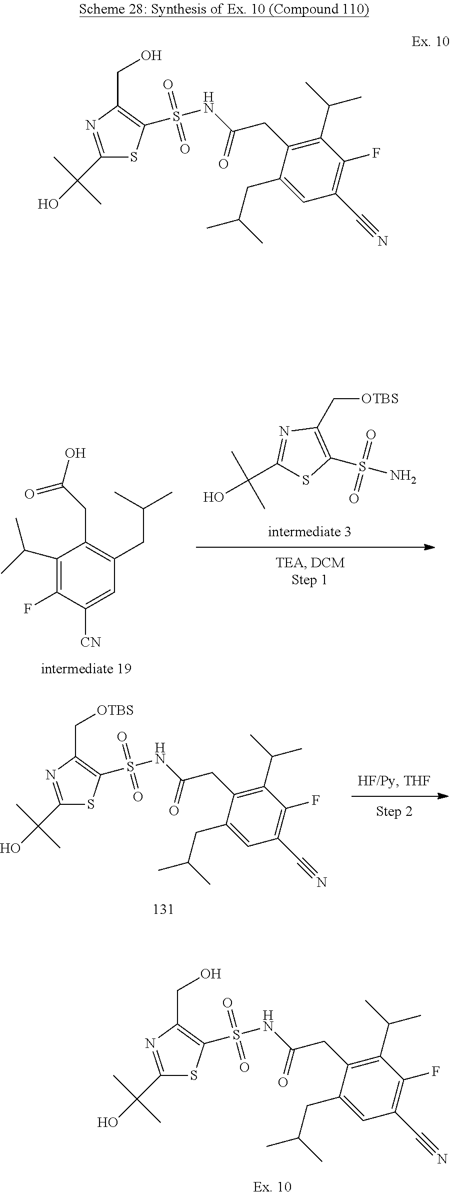 [SMILES: CC(C)Cc1cc(C#N)c(F)c(C(C)C)c1CC(=O)NS(=O)(=O)c1sc(C(C)(C)O)nc1CO ; CC(C)Cc1cc(C#N)c(F)c(C(C)C)c1CC(=O)O ; CC(C)(O)c1nc(CO[Si](C)(C)C(C)(C)C)c(S(N)(=O)=O)s1 ; CC(C)Cc1cc(C#N)c(F)c(C(C)C)c1CC(=O)NS(=O)(=O)c1sc(C(C)(C)O)nc1CO[Si](C)(C)C(C)(C)C ; "HF/Py, THF" ; CC(C)Cc1cc(C#N)c(F)c(C(C)C)c1CC(=O)NS(=O)(=O)c1sc(C(C)(C)O)nc1CO]

### 1. Synthesis of N-[(4-[[(tert-butyldimethylsilyl)oxy]methyl]-2-(2-hydroxypropan-2-yl)-1,3-thiazol-5-yl)sulfonyl]-2-[4-cyano-3-fluoro-6-(2-methylpropyl)-2-(propan-2-yl)phenyl]acetamide

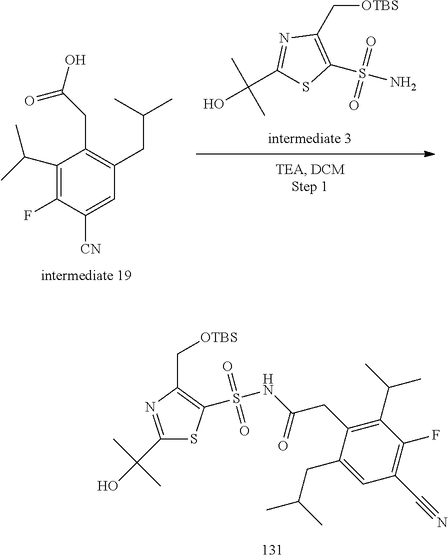 [SMILES: CC(C)Cc1cc(C#N)c(F)c(C(C)C)c1CC(=O)O ; CC(C)(O)c1nc(CO[Si](C)(C)C(C)(C)C)c(S(N)(=O)=O)s1 ; CC(C)Cc1cc(C#N)c(F)c(C(C)C)c1CC(=O)NS(=O)(=O)c1sc(C(C)(C)O)nc1CO[Si](C)(C)C(C)(C)C]

A solution/mixture of 2-[4-cyano-3-fluoro-6-(2-methylpropyl)-2-(propan-2-yl)phenyl]acetic acid (100 mg, 360 mmol, 1 equiv), DMF (0.02 mL, 0.26 mmol, 0.72 equiv) and (COCl)₂ (137.3 mg, 1.08 mmol, 3.00 equiv) in DCM (10 mL) was stirred for 30 min at room temperature. The resulting mixture was concentrated under vacuum. This resulted in 2-[4-cyano-3-fluoro-6-(2-methylpropyl)-2-(propan-2-yl)phenyl]acetyl chloride (100 mg, crude) as a yellow solid.

To a stirred solution/mixture of 4-[[(tert-butyldimethylsilyl)oxy]methyl]-2-(2-hydroxypropan-2-yl)-1,3-thiazole-5-sulfonamide (111.5 mg, 0.30 mmol, 0.9 equiv) and TEA (102.6 mg, 1.01 mmol, 3 equiv) in DCM (10 mL) was added 2-[4-cyano-3-fluoro-6-(2-methylpropyl)-2-(propan-2-yl)phenyl]acetyl chloride (100 mg, 340 mmol, 1 equiv) at room temperature. The resulting mixture was stirred for 3 h at room temperature. The resulting mixture was concentrated under vacuum. This resulted in N-[(4-[[(tert-butyldimethylsilyl)oxy]methyl]-2-(2-hydroxypropan-2-yl)-1,3-thiazol-5-yl)sulfonyl]-2-[4-cyano-3-fluoro-6-(2-methylpropyl)-2-(propan-2-yl)phenyl]acetamide (150 mg, crude) as a yellow oil.

LC-MS—N-[(4-[[(tert-butyldimethylsilyl)oxy]methyl]-2-(2-hydroxypropan-2-yl)-1,3-thiazol-5-yl)sulfonyl]-2-[4-cyano-3-fluoro-6-(2-methylpropyl)-2-(propan-2-yl)phenyl]acetamide (Method B): (ES, m/z): [M+H]⁺=626.3, retention time: 1.920 min.

### 2. Synthesis of 2-[4-cyano-3-fluoro-6-(2-methylpropyl)-2-(propan-2-yl)phenyl]-N-[[4-(hydroxymethyl)-2-(2-hydroxypropan-2-yl)-1,3-thiazol-5-yl]sulfonyl]acetamide

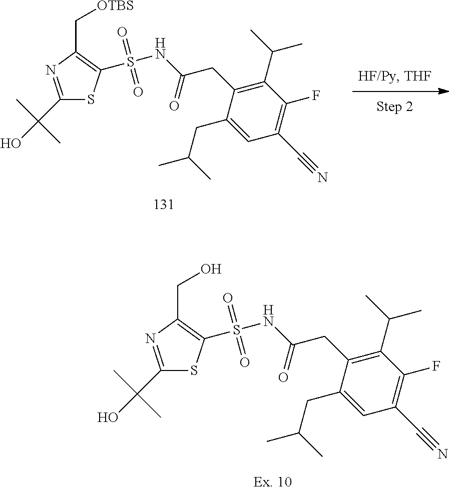 [SMILES: CC(C)Cc1cc(C#N)c(F)c(C(C)C)c1CC(=O)NS(=O)(=O)c1sc(C(C)(C)O)nc1CO[Si](C)(C)C(C)(C)C ; "HF/Py, THF" ; CC(C)Cc1cc(C#N)c(F)c(C(C)C)c1CC(=O)NS(=O)(=O)c1sc(C(C)(C)O)nc1CO]

Into a 50-mL round-bottom flask, was placed N-[(4-[[(tert-butyldimethylsilyl)oxy]methyl]-2-(2-hydroxypropan-2-yl)-1,3-thiazol-5-yl)sulfonyl]-2-[4-cyano-3-fluoro-6-(2-methylpropyl)-2-(propan-2-yl)phenyl]acetamide (150 mg, 0.24 mmol, 1 equiv), THE (10 mL), and HF/Py (239.7 mg, 2.40 mmol, 10.00 equiv). The resulting solution was stirred for 1 h at room temperature. The resulting mixture was concentrated. The crude product was purified by Prep-HPLC with the following conditions (2#SHIMADZU (HPLC-01)): Column, XBridge Shield RP18 OBD Column, 19*250 mm, 10 um; mobile phase, water (10 mmol/L NH₄HCO₃) and ACN (25% PhaseB up to 45% in 10 min); Detector, UV 254/210 nm. This resulted in 69.1 mg (56.35%) of 2-[4-cyano-3-fluoro-6-(2-methylpropyl)-2-(propan-2-yl)phenyl]-N-[[4-(hydroxymethyl)-2-(2-hydroxypropan-2-yl)-1,3-thiazol-5-yl]sulfonyl]acetamide as a white solid.

LC-MS-2-[4-cyano-3-fluoro-6-(2-methylpropyl)-2-(propan-2-yl)phenyl]-N-[[4-(hydroxymethyl)-2-(2-hydroxypropan-2-yl)-1,3-thiazol-5-yl]sulfonyl]acetamide(Method N): (ES, m/z): [M−H]⁻=510.2, retention time: 1.267.

H-NMR-2-[4-cyano-3-fluoro-6-(2-methylpropyl)-2-(propan-2-yl)phenyl]-N-[[4-(hydroxymethyl)-2-(2-hydroxypropan-2-yl)-1,3-thiazol-5-yl]sulfonyl]acetamide: ¹H NMR (300 MHz, CD₃OD-d₄) δ7.34 (d, J=6.4 Hz, 1H), 4.90-4.89 (d, J=3 Hz, 2H), 3.82 (s, 2H), 2.99-2.87 (m, 1H), 2.47 (d, J=7.3 Hz, 2H), 1.71-1.64 (m, 1H), 1.60 (s, 6H), 1.20 (m, 6H), 0.88 (d, J=6.6 Hz, 6H).

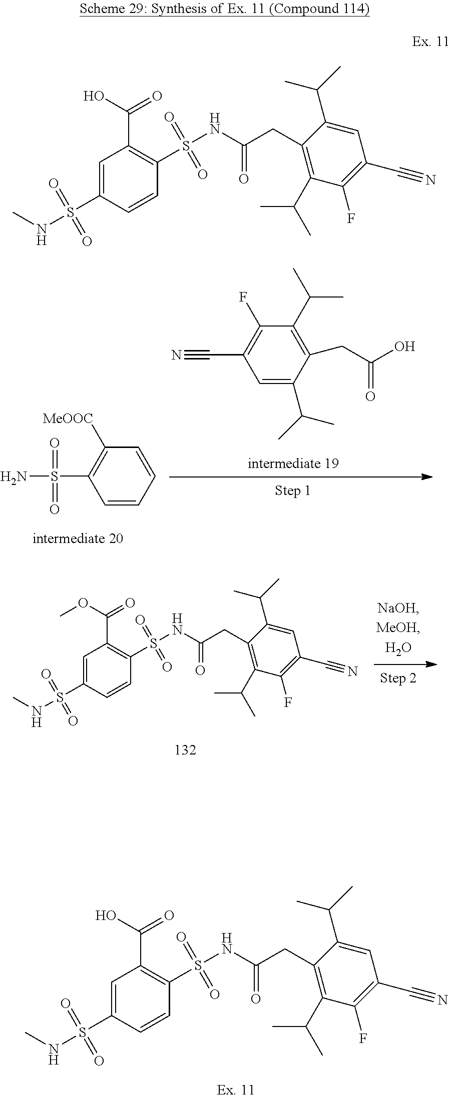

### 1. Synthesis of methyl 2-([2-[4-cyano-3-fluoro-2,6-bis(propan-2-yl)phenyl]ethane]sulfonyl)-5-(methylsulfamoyl)benzoate

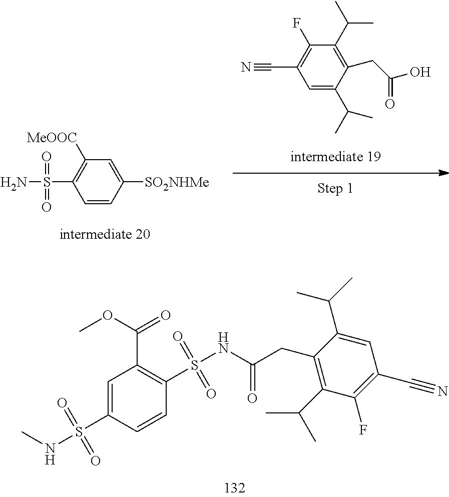

Into a 100-mL round-bottom flask, was placed methyl 5-(methylsulfamoyl)-2-sulfamoylbenzoate (300 mg, 1 equiv), HATU (1.1 g, 3 equiv), DIEA (380 mg, 3 equiv), and DCM (15 mL). The resulting solution was stirred for 12 hr at room temperature, after which it was concentrated. The crude product was purified by Prep-HPLC. This resulted in 90 mg of methyl 2-([2-[4-cyano-3-fluoro-2,6-bis(propan-2-yl)phenyl]ethane]sulfonyl)-5-(methylsulfamoyl)benzoate as a white solid

### 2. Synthesis of 2-([2-[4-cyano-3-fluoro-2,6-bis(propan-2-yl)phenyl]acetamido]sulfonyl)-5-(methylsulfamoyl)benzoic acid

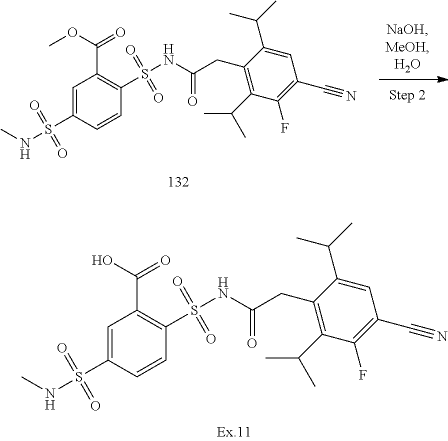 [SMILES: CNS(=O)(=O)c1ccc(S(=O)(=O)NC(=O)Cc2c(C(C)C)cc(C#N)c(F)c2C(C)C)c(C(=O)OC)c1 ; CNS(=O)(=O)c1ccc(S(=O)(=O)NC(=O)Cc2c(C(C)C)cc(C#N)c(F)c2C(C)C)c(C(=O)O)c1]

Into a 100-mL round-bottom flask, was placed methyl 2-([2-[4-cyano-3-fluoro-2,6-bis(propan-2-yl)phenyl]acetamido]sulfonyl)-5-(methylsulfamoyl)benzoate (50 mg), NaOH (20 mg), MeOH (10 mL), and H₂O (10 mL). The resulting solution was stirred for 12 hours. The crude product was purified by Prep-HPLC. This resulted in 17.8 mg of 2-([2-[4-cyano-3-fluoro-2,6-bis(propan-2-yl)phenyl]acetamido]sulfonyl)-5-(methylsulfamoyl)benzoic acid as a white solid.

LC-MS-2-([2-[4-cyano-3-fluoro-2,6-bis(propan-2-yl)phenyl]acetamido]sulfonyl)-5-(methylsulfamoyl)benzoic acid(Method N): (ES, m/z): [M−H]⁻=538.1, retention time: 0.992.

H-NMR-2-([2-[4-cyano-3-fluoro-2,6-bis(propan-2-yl)phenyl]acetamido]sulfonyl)-5-(methylsulfamoyl)benzoic acid: (MeOD, ppm): ¹H NMR (300 MHz, MeOD) δ 8.228 (m, 1H), 8.127-8.067 (s, 1H), 7.909-7.801 (s, 1H), 7.404-7.803 (m, 1H), 3.823 (s, 2H), 2.663 (s, 6H), 1.405-1.145 (m, 12H).

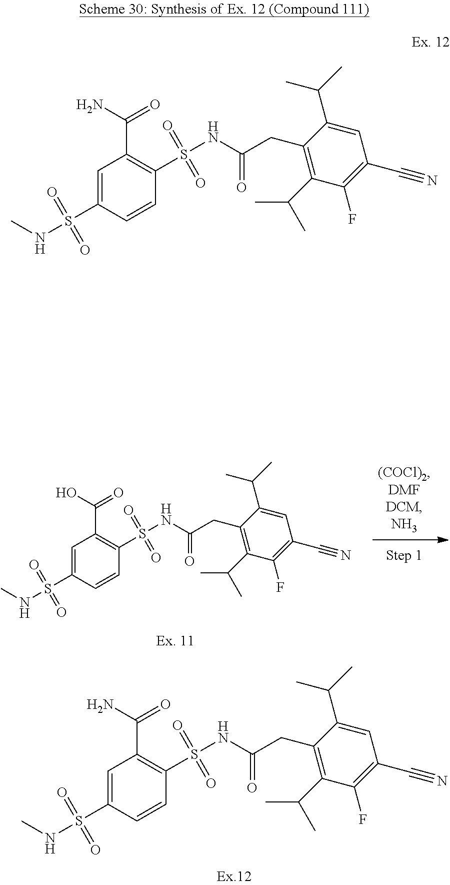 [SMILES: CNS(=O)(=O)c1ccc(S(=O)(=O)NC(=O)Cc2c(C(C)C)cc(C#N)c(F)c2C(C)C)c(C(N)=O)c1 ; CNS(=O)(=O)c1ccc(S(=O)(=O)NC(=O)Cc2c(C(C)C)cc(C#N)c(F)c2C(C)C)c(C(=O)O)c1 ; CNS(=O)(=O)c1ccc(S(=O)(=O)NC(=O)Cc2c(C(C)C)cc(C#N)c(F)c2C(C)C)c(C(N)=O)c1]

### 1. Synthesis of 2-([2-[4-cyano-3-fluoro-2,6-bis(propan-2-yl)phenyl]acetamido]sulfonyl)-5-(methylsulfamoyl)benzamide

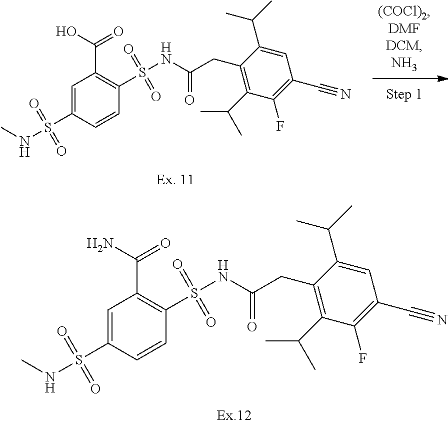 [SMILES: CNS(=O)(=O)c1ccc(S(=O)(=O)NC(=O)Cc2c(C(C)C)cc(C#N)c(F)c2C(C)C)c(C(=O)O)c1 ; CNS(=O)(=O)c1ccc(S(=O)(=O)NC(=O)Cc2c(C(C)C)cc(C#N)c(F)c2C(C)C)c(C(N)=O)c1]

The acid chloride was prepared from Ex. 11 by reacting with oxalyly chloride (5 eq). DMF (3 drops) was added and stirred at rt for 3 h. The solution was rotavaped and the crude pdt was used as is in the next step.

Into a 50-mL round-bottom flask, was placed 2-([2-[4-cyano-3-fluoro-2,6-bis(propan-2-yl)phenyl]acetamido]sulfonyl)-5-(methylsulfamoyl)benzoyl chloride (50 mg), (COCl)₂ (0.5 mL), DMF (one drop), and DCM (10 mL). After 30 minutes, NH3/THF (20 mL) was added. The resulting solution was stirred for 2 hours at room temperature. The crude product was purified by Prep-HPLC. This resulted in 13.7 mg of 2-([2-[4-cyano-3-fluoro-2,6-bis(propan-2-yl)phenyl]acetamido]sulfonyl)-5-(methylsulfamoyl)benzamide as a white solid.

LC-MS-2-([2-[4-cyano-3-fluoro-2,6-bis(propan-2-yl)phenyl]acetamido]sulfonyl)-5-(methylsulfamoyl)benzamide(Method N): (ES, m/z): [M−H]⁻=537.1, retention time: 1.252

H-NMR-2-([2-[4-cyano-3-fluoro-2,6-bis(propan-2-yl)phenyl]acetamido]sulfonyl)-5-(methylsulfamoyl)benzamide: (MeOD, ppm): ¹H NMR (300 MHz, MeOD) δ8.343-8.316 (m, 1H), 8.033-7.995 (m, 2H), 7.466-7.445. (m, 1H), 3.957-3.902 (s, 2H), 3.059-2.969 (m, 2H), 2.573 (s, 3H), 1.139-1.093 (m, 12H).

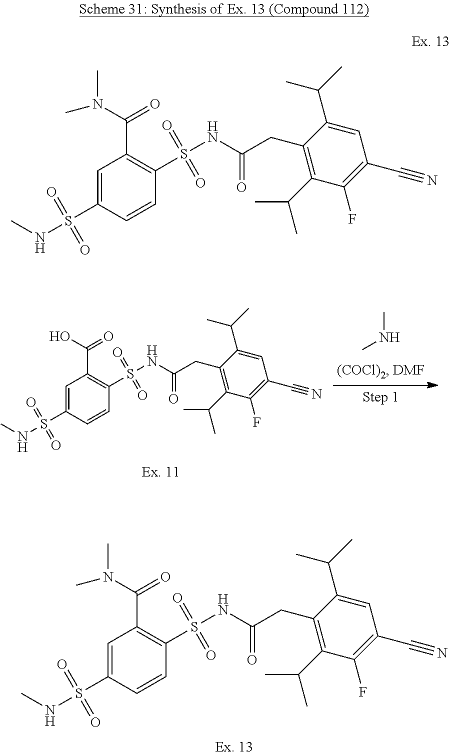

### 1. Synthesis of 2-([2-[4-cyano-3-fluoro-2,6-bis(propan-2-yl)phenyl]acetamido]sulfonyl)-N,N-dimethyl-5-(methylsulfamoyl)benzamide

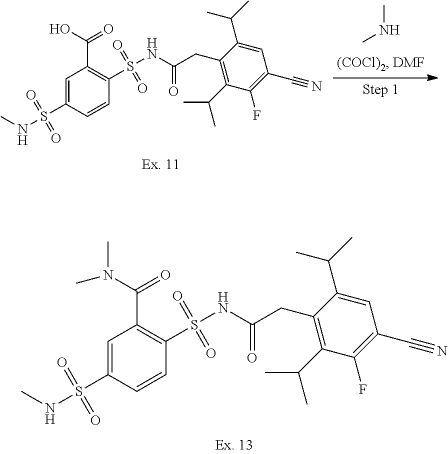

Into a 50-mL round-bottom flask, was placed 2-([2-[4-cyano-3-fluoro-2,6-bis(propan-2-yl)phenyl]acetamido]sulfonyl)-5-(methylsulfamoyl)benzoyl chloride (50 mg), (COCl)2 (0.5 mL), DMF (one drop), and DCM (10 mL). After 30 minutes, (CH₃)₂NH/THF (10 mL) was added. The resulting solution was stirred for 3 hours. The crude product was purified by Prep-HPLC. This resulted in 40 mg of 2-([2-[4-cyano-3-fluoro-2,6-bis(propan-2-yl)phenyl]acetamido]sulfonyl)-N,N-dimethyl-5-(methylsulfamoyl)benzamide as a white solid

LC-MS-2-([2-[4-cyano-3-fluoro-2,6-bis(propan-2-yl)phenyl]acetamido]sulfonyl)-N,N-dimethyl-5-(methylsulfamoyl)benzamide(Method N): (ES, m/z): [M+H]⁺=567.3, retention time: 1.280.

H-NMR-2-([2-[4-cyano-3-fluoro-2,6-bis(propan-2-yl)phenyl]acetamido]sulfonyl)-N,N-dimethyl-5-(methylsulfamoyl)benzamide: (MeOD, ppm): ¹H NMR (300 MHz, MeOD) δ8.256-8.235 (m, 1H), 7.936-7.915 (m, 1H), 7.740 (s, 1H), 7.413-7.398 (s, 1H), 3.896-3.800 (m, 2H), 3.134-3.056 (m, 5H), 2.862 (s, 3H), 2.538 (s, 3H), 1.253-0.928 (m, 12H).

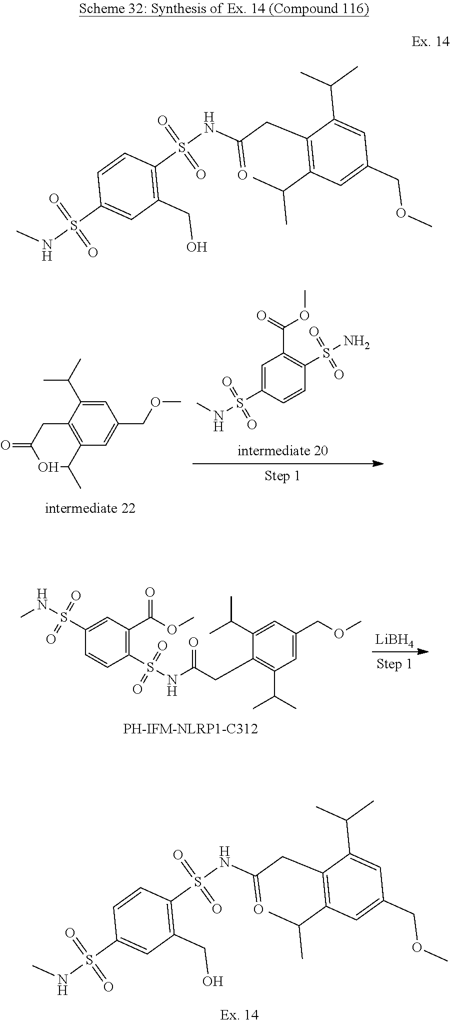

### 1. Synthesis of 2-([2-[4-(methoxymethyl)-2,6-bis(propan-2-yl)phenyl]acetamido]sulfonyl)-5-(methylsulfamoyl)benzoate

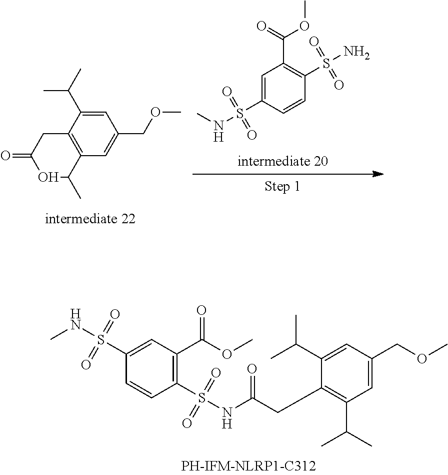

Into a 100-mL round-bottom flask, was placed methyl 5-(methylsulfamoyl)-2-sulfamoylbenzoate (50 mg), 2-[4-(methoxymethyl)-2,6-bis(propan-2-yl)phenyl]acetyl chloride (prepared by method similar to as described above) (55 mg), TEA (40 mg), DCM (15 mL). The resulting solution was stirred for 2 hr at rt. The crude product was purified by Prep-HPLC. This resulted in 10 mg of methyl 2-([2-[4-(methoxymethyl)-2,6-bis(propan-2-yl)phenyl]acetamido]sulfonyl)-5-(methylsulfamoyl)benzoate as a white solid.

LC-MS-2-([2-[4-(methoxymethyl)-2,6-bis(propan-2-yl)phenyl]acetamido]sulfonyl)-5-(methylsulfamoyl)benzoate: [M+H]⁺=555, retention time: 2.040 Method: Kinetex EVO, C18, 3×50 mm, 2.2 um column, 1.0 uL injection, 1.0 mL/min flow rate, 90-900 amu scan range, 190-400 nm UV range, Mobile phase A: Water (5 mmoL/L NH4HCO3) and Mobile Phase B: MeCN. 10% MPB to 60.0% in 2.9 min, 60% MPB to 95% in 0.4 min, hold at 95% MPB for 0.4 min, 95% MPB to 10% in 0.1 min, then equilibration to 10% MPB for 0.14 min.

H-NMR-2-([2-[4-(methoxymethyl)-2,6-bis(propan-2-yl)phenyl]acetamido]sulfonyl)-5-(methylsulfamoyl)benzoate: (DMSO, ppm): ¹H NMR (300 MHz, DMSO) δ 12.71 (s, 1H), 8.22-7.76 (m, 3H), 6.94 (s, 2H), 4.29 (s, 2H), 3.91 (s, 3H), 3.70 (s, 2H), 3.26 (s, 3H), 2.94-2.73 (s, 2H), 2.27 (s, 4H), 0.98-0.96 (m, 12H).

### 2. Synthesis of 2-([2-[4-(methoxymethyl)-2,6-bis(propan-2-yl)phenyl]acetamido]sulfonyl)-5-(methylsulfamoyl)benzoate

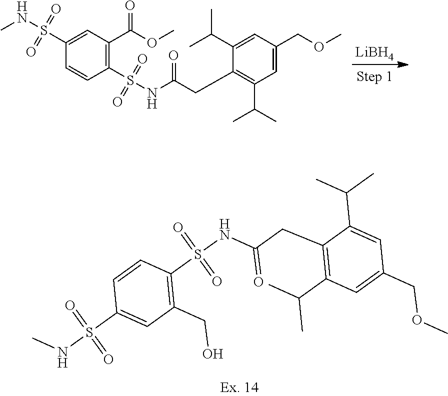

Into a 50-mL round-bottom flask, was placed methyl 2-([2-[4-(methoxymethyl)-2,6-bis(propan-2-yl)phenyl]acetamido]sulfonyl)-5-(methylsulfamoyl)benzoate (30 mg), LiBH4 (10 mg), and THE (10 mL). The resulting solution was stirred for 3 hr at rt. The crude product was purified by Prep-HPLC. This resulted in 5 mg of N-[2-(hydroxymethyl)-4-(methylsulfamoyl)benzenesulfonyl]-2-[4-(methoxymethyl)-2,6-bis(propan-2-yl)phenyl]acetamide as a white solid.

LC-MS-N-[2-(hydroxymethyl)-4-(methylsulfamoyl)benzenesulfonyl]-2-[4-(methoxymethyl)-2,6-bis(propan-2-yl)phenyl]acetamide: (ES, m/z): [M+H]⁺=527, retention time: 1.035 Method: Kinetex EVO, C18, 3×50 mm, 2.2 um column, 1.0 uL injection, 1.5 mL/min flow rate, 90-900 amu scan range, 190-400 nm UV range, 10-95% (2.1 min), 95% (0.6 min) gradient with ACN and water (0.5% NH4HCO3), 3 minute total run time.

H-NMR—N-[2-(hydroxymethyl)-4-(methylsulfamoyl)benzenesulfonyl]-2-[4-(methoxymethyl)-2,6-bis(propan-2-yl)phenyl]acetamide: (DMSO, ppm): 1H NMR (300 MHz, DMSO) δ 8.11 (s, 2H), 8.00-7.97 (m, 1H), 7.67-7.64 (m, 2H), 5.50 (s, 1H), 4.97 (s, 2H), 4.27 (s, 2H), 3.61 (s, 2H), 3.23 (s, 2H), 2.91 (s. 2H), 2.38-2.36 (m, 3H), 0.98-0.96 (m, 12H).

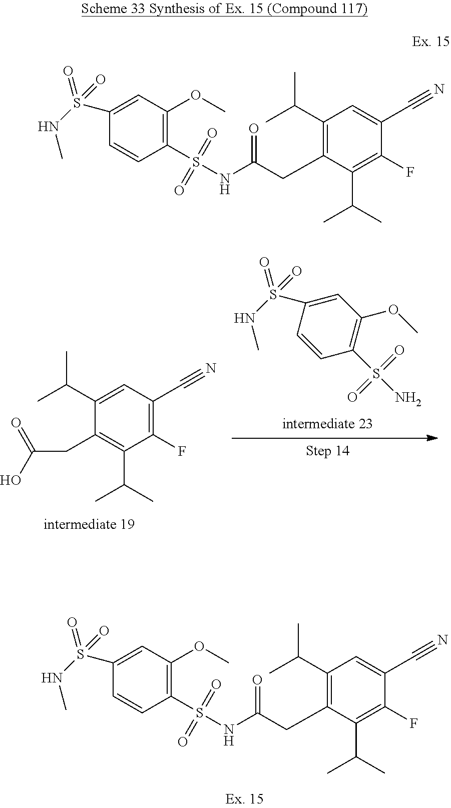 [SMILES: CNS(=O)(=O)c1ccc(S(=O)(=O)NC(=O)Cc2c(C(C)C)cc(C#N)c(F)c2C(C)C)c(OC)c1 ; CC(C)c1cc(C#N)c(F)c(C(C)C)c1CC(=O)O ; CNS(=O)(=O)c1ccc(S(N)(=O)=O)c(OC)c1 ; CNS(=O)(=O)c1ccc(S(=O)(=O)NC(=O)Cc2c(C(C)C)cc(C#N)c(F)c2C(C)C)c(OC)c1]

### 1. Synthesis of 2-(4-cyano-3-fluoro-2,6-diisopropylphenyl)-N-(2-methoxy-4-(N-methylsulfamoyl)phenylsulfonyl)acetamide

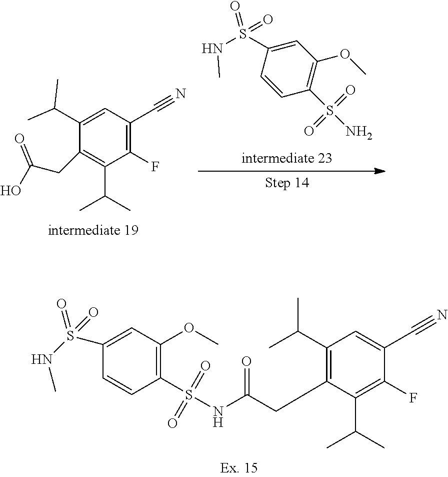

Into a 50-mL round-bottom flask, was placed a solution of 2-(4-cyano-2,6-diisopropylphenyl)acetic acid (218 mg, 0.83 mmol, 1.0 equiv) in DCM (10 mL). To the solution was added (COCl)₂ (209 mg, 1.66 mmol, 2.0 equiv) dropwise at 0° C., and the mixture was stirred at room temperature for 2 h. The crude was concentrated in vacuo. The resulting solid (2-(4-cyano-3-fluoro-2,6-diisopropylphenyl)acetyl chloride) (220 mg) was used directly for the next step.

Into a 50-mL round-bottom flask, was placed a solution of 3-methoxy-N1-methylbenzene-1,4-disulfonamide (232 mg, 0.83 mmol, 1.0 equiv) in THE (10 mL). NaH (50 mg, 1.25 mmol, 1.5 equiv, 60%) was added to the solution in portions at 0° C. To the solution was added freshly prepared 2-(4-cyano-3-fluoro-2,6-diisopropylphenyl)acetyl chloride (220 mg) in THE (5 mL) dropwise at 0° C. The mixture was stirred at room temperature for 2 h. The reaction was quenched with ice-water (20 mL), extracted with EtOAc (50 mL*3). The combined organic phase was dried over Na₂SO₄ and concentrated and purified with Flash-Prep-HPLC under the following conditions (IntelFlash-1): Column, C18 silica gel; mobile phase, MeCN/H₂O=10/90 increasing to MeCN/H₂O=90/10 within 1 hr; Detector, UV254. 500 mL product was obtained. This resulted in 94 mg (21.5% for 2 steps) of 2-(4-cyano-3-fluoro-2,6-diisopropylphenyl)-N-(2-methoxy-4-(N-methylsulfamoyl)phenylsulfonyl)acetamide as a white solid.

LCMS of 2-(4-cyano-3-fluoro-2,6-diisopropylphenyl)-N-(2-methoxy-4-(N-methylsulfamoyl)phenylsulfonyl)acetamide (Method L): 524.0 [M−H]⁻, retention time 1.037 min.

1H NMR of 2-(4-cyano-3-fluoro-2,6-diisopropylphenyl)-N-(2-methoxy-4-(N-methylsulfamoyl)phenylsulfonyl)acetamide (300 MHz, Methanol-d4) δ 8.10 (d, J=8.2 Hz, 1H), 7.58 (d, J=1.6 Hz, 1H), 7.49 (dd, J=8.2, 1.6 Hz, 1H), 7.42 (d, J=6.3 Hz, 1H), 4.08 (s, 3H), 3.86 (s, 2H), 2.93 (m, 1H), 2.53 (s, 3H), 1.21-1.07 (m, 12H).

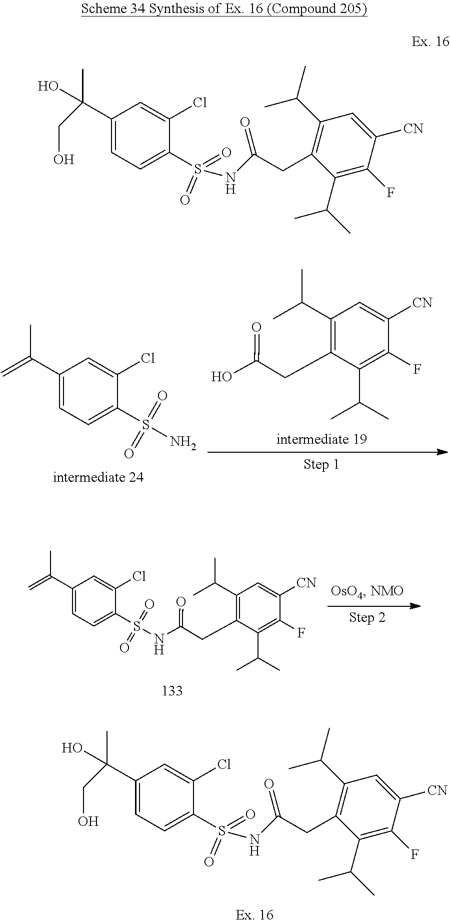 [SMILES: CC(C)c1cc(C#N)c(F)c(C(C)C)c1CC(=O)NS(=O)(=O)c1ccc(C(C)(O)CO)cc1Cl ; C=C(C)c1ccc(S(N)(=O)=O)c(Cl)c1 ; CC(C)c1cc(C#N)c(F)c(C(C)C)c1CC(=O)O ; C=C(C)c1ccc(S(=O)(=O)NC(=O)Cc2c(C(C)C)cc(C#N)c(F)c2C(C)C)c(Cl)c1 ; CC(C)c1cc(C#N)c(F)c(C(C)C)c1CC(=O)NS(=O)(=O)c1ccc(C(C)(O)CO)cc1Cl]

### 1. Synthesis of N-((2-chloro-4-(prop-1-en-2-yl)phenyl)sulfonyl)-2-(4-cyano-3-fluoro-2,6-diisopropylphenyl)acetamide

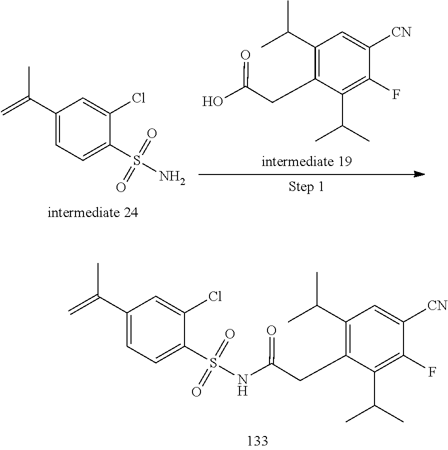 [SMILES: C=C(C)c1ccc(S(N)(=O)=O)c(Cl)c1 ; CC(C)c1cc(C#N)c(F)c(C(C)C)c1CC(=O)O ; C=C(C)c1ccc(S(=O)(=O)NC(=O)Cc2c(C(C)C)cc(C#N)c(F)c2C(C)C)c(Cl)c1]

To a stirred solution of 2-chloro-4-(prop-1-en-2-yl)benzene-1-sulfonamide (80 mg, 0.35 mmol, 1 equiv) and 2-[4-cyano-3-fluoro-2,6-bis(propan-2-yl)phenyl]acetic acid (90.9 mg, 0.35 mmol, 1 equiv) in DMF (5 mL) were added TEA (104.8 mg, 1.04 mmol, 3 equiv) and HATU (196.9 mg, 0.52 mmol, 1.5 equiv) in portions at room temperature under nitrogen atmosphere. The resulting mixture was stirred for overnight at room temperature under nitrogen atmosphere. The resulting mixture was concentrated under vacuum. The residue was purified by silica gel column chromatography, eluted with PE/EtOAc (1:1) to afford N-[[2-chloro-4-(prop-1-en-2-yl)benzene]sulfonyl]-2-[4-cyano-3-fluoro-2,6-bis(propan-2-yl)phenyl]acetamide(100 mg, 60.72%) as a white solid.

### 2. Synthesis of N-[[2-chloro-4-(1,2-dihydroxypropan-2-yl)benzene]sulfonyl]-2-[4-cyano-3-fluoro-2,6-bis(propan-2-yl)phenyl]acetamide

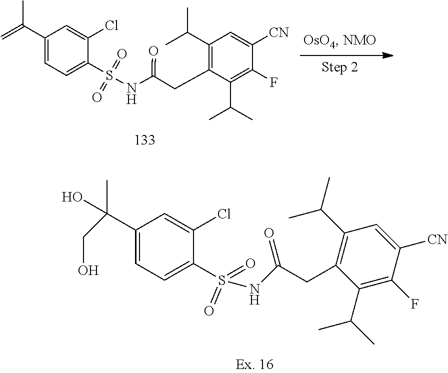 [SMILES: C=C(C)c1ccc(S(=O)(=O)NC(=O)Cc2c(C(C)C)cc(C#N)c(F)c2C(C)C)c(Cl)c1 ; CC(C)c1cc(C#N)c(F)c(C(C)C)c1CC(=O)NS(=O)(=O)c1ccc(C(C)(O)CO)cc1Cl]

To a stirred solution of N-[[2-chloro-4-(prop-1-en-2-yl)benzene]sulfonyl]-2-[4-cyano-3-fluoro-2,6-bis(propan-2-yl)phenyl]acetamide(100 mg, 0.21 mmol, 1 equiv) in H₂O(2 mL), acetone (2 mL) and t-BuOH (3 mL) were added tetraoxoosmium (5.3 mg, 0.02 mmol, 0.1 equiv) and NMO (73.7 mg, 0.63 mmol, 3 equiv) dropwise/in portions at room temperature under nitrogen atmosphere. The resulting mixture was stirred for 3 h at room temperature under nitrogen atmosphere. The resulting mixture was concentrated under reduced pressure. The crude residue was redissolved in cold meoh (5 mL) and filtered; the filter cake was washed with MeOH (3×10 mL). The filtrate was concentrated under reduced pressure. Then the crude product (5 mL) was purified by Prep-HPLC with the following conditions, Column: XBridge Shield RP18 OBD Column 19*250 mm, 10 um; Mobile Phase A: water (10 MMOL/L NH4HCO3), Mobile Phase B: ACN; Flow rate: 25 mL/min; Gradient: 17% B to 44% B in 8 min; 254/210 nm; Rt: 7.33 min. This resulted in N-[[2-chloro-4-(1,2-dihydroxypropan-2-yl)benzene]sulfonyl]-2-[4-cyano-3-fluoro-2,6-bis(propan-2-yl)phenyl]acetamide (37 mg, 34.54%) as a white solid.

LCMS of N-[[2-chloro-4-(1,2-dihydroxypropan-2-yl)benzene]sulfonyl]-2-[4-cyano-3-fluoro-2,6-bis(propan-2-yl)phenyl]acetamide (Method K): 509.1 [M−H]⁻, retention time 1.245 min.

H-NMR-N-[[2-chloro-4-(1,2-dihydroxypropan-2-yl)benzene]sulfonyl]-2-[4-cyano-3-fluoro-2,6-bis(propan-2-yl)phenyl]acetamide: ¹H NMR (300 MHz, DMSO-d₆) δ 7.89 (d, J=8.2 Hz, 1H), 7.58 (d, J=6.0 Hz, 2H), 7.46 (d, J=8.5 Hz, 1H), 5.20 (s, 1H), 4.82 (t, J=5.7 Hz, 1H), 3.76 (s, 2H), 3.41 (d, J=4.8 Hz, 2H), 3.10 (s, 2H), 1.36 (s, 3H), 1.11 (dd, J=17.9, 6.8 Hz, 12H).

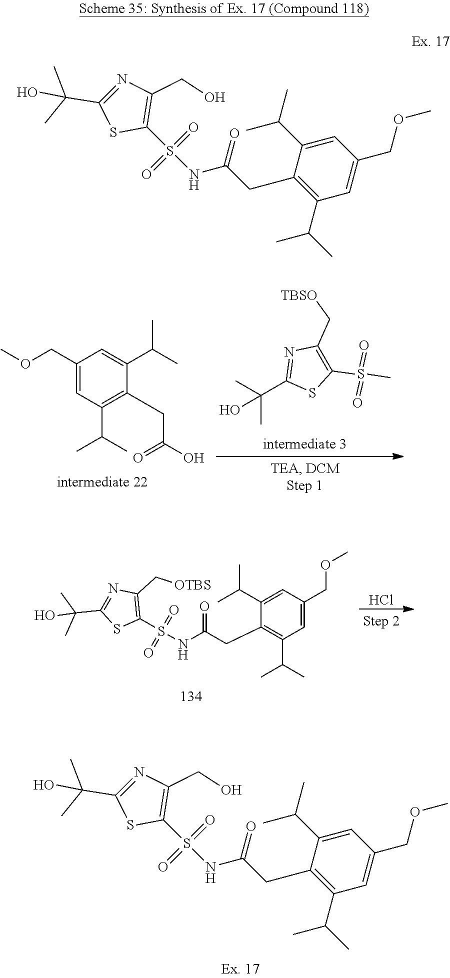

### 1. Synthesis of N-(4-[[(tert-butyldimethylsilyl)oxy]methyl]-2-(2-hydroxypropan-2-yl)-1,3-thiazole-5-sulfonyl)-2-[4-(methoxymethyl)-2,6-bis(propan-2-yl)phenyl]acetamide

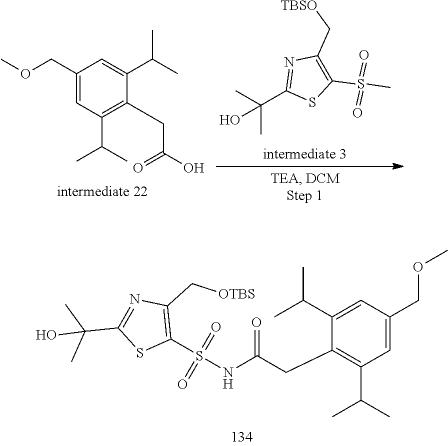 [SMILES: COCc1cc(C(C)C)c(CC(=O)O)c(C(C)C)c1 ; CC(C)(O)c1nc(CO[Si](C)(C)C(C)(C)C)c(S(C)(=O)=O)s1 ; COCc1cc(C(C)C)c(CC(=O)NS(=O)(=O)c2sc(C(C)(C)O)nc2CO[Si](C)(C)C(C)(C)C)c(C(C)C)c1]

To a stirred solution/mixture of 2-[4-(methoxymethyl)-2,6-bis(propan-2-yl)phenyl]acetic acid (60 mg, 0.23 mmol, 1 equiv) and DMF (0.02 mL, 0.001 equiv) in DCM (8 mL) were added (COCl)₂ (86.4 mg, 0.68 mmol, 3 equiv) dropwise at room temperature. The resulting mixture was stirred for 0.5 h at room temperature. The resulting mixture was concentrated under vacuum. This resulted in 2-[4-(methoxymethyl)-2,6-bis(propan-2-yl)phenyl]acetyl chloride (70 mg, crude) as a yellow solid.

To a stirred solution/mixture of 4-[[(tert-butyldimethylsilyl)oxy]methyl]-2-(2-hydroxypropan-2-yl)-1,3-thiazole-5-sulfonamide (64 mg, 0.17 mmol, 1 equiv) and TEA (53.0 mg, 0.52 mmol, 3 equiv) in DCM(10 mL) were added 2-[4-(methoxymethyl)-2,6-bis(propan-2-yl)phenyl]acetyl chloride (64.2 mg, 0.23 mmol, 1.300 equiv) at room temperature. The resulting mixture was stirred for 4 h at room temperature. The resulting mixture was concentrated under vacuum. This resulted in N-(4-[[(tert-butyldimethylsilyl)oxy]methyl]-2-(2-hydroxypropan-2-yl)-1,3-thiazole-5-sulfonyl)-2-[4-(methoxymethyl)-2,6-bis(propan-2-yl)phenyl]acetamide(150 mg, crude) as a yellow solid.

LCMS of N-(4-[[(tert-butyldimethylsilyl)oxy]methyl]-2-(2-hydroxypropan-2-yl)-1,3-thiazole-5-sulfonyl)-2-[4-(methoxymethyl)-2,6-bis(propan-2-yl)phenyl]acetamide (Method B): 613.3 [M+H]⁺, retention time 0.980 min.

### 2. Synthesis of N-[4-(hydroxymethyl)-2-(2-hydroxypropan-2-yl)-1,3-thiazole-5-sulfonyl]-2-[4-(methoxymethyl)-2,6-bis(propan-2-yl)phenyl]acetamide

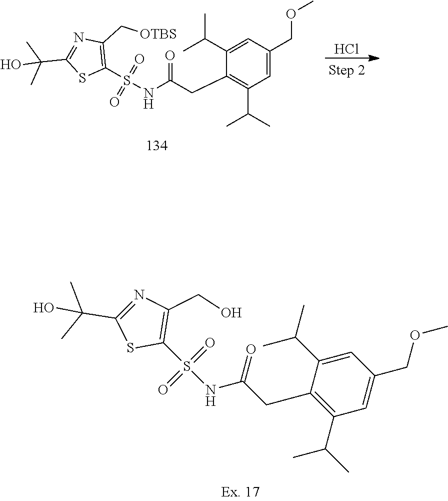 [SMILES: COCc1cc(C(C)C)c(CC(=O)NS(=O)(=O)c2sc(C(C)(C)O)nc2CO[Si](C)(C)C(C)(C)C)c(C(C)C)c1 ; Cl ; COCc1cc(C(C)C)c(CC(=O)NS(=O)(=O)c2sc(C(C)(C)O)nc2CO)c(C(C)C)c1]

Into a 25-mL round-bottom flask, was placed N-(4-[[(tert-butyldimethylsilyl)oxy]methyl]-2-(2-hydroxypropan-2-yl)-1,3-thiazole-5-sulfonyl)-2-[4-(methoxymethyl)-2,6-bis(propan-2-yl)phenyl]acetamide (150 mg, 0.24 mmol, 1 equiv) in DCM (10 mL) and HCl/dioxane (5 mL). The resulting solution was stirred for 1 h at room temperature. The resulting mixture was concentrated. The crude product was purified by Prep-HPLC under the following conditions (Prep-HPLC-018): Column, XBridge C18 OBD Prep Column, 100 A, 19 mm×250 mm; mobile phase, Water (10 mmol/L NH4HCO3) and ACN (10% PhaseB up to 53% in 7 min); Detector, UV. This resulted in 27.8 mg (22.78%) of N-[4-(hydroxymethyl)-2-(2-hydroxypropan-2-yl)-1,3-thiazole-5-sulfonyl]-2-[4-(methoxymethyl)-2,6-bis(propan-2-yl)phenyl]acetamide as a white solid.

LCMS of N-[4-(hydroxymethyl)-2-(2-hydroxypropan-2-yl)-1,3-thiazole-5-sulfonyl]-2-[4-(methoxymethyl)-2,6-bis(propan-2-yl)phenyl]acetamide (Method K): 497.1 [M−H]⁻, retention time 0.766 min.

H-NMR—N-[4-(hydroxymethyl)-2-(2-hydroxypropan-2-yl)-1,3-thiazole-5-sulfonyl]-2-[4-(methoxymethyl)-2,6-bis(propan-2-yl)phenyl]acetamide: ¹H NMR (400 MHz, CD₃OD-d₄) δ 7.06 (s, 2H), 4.89 (s, 2H), 4.40 (s, 2H), 3.75 (s, 2H), 3.36 (m, 3H), 3.07 (m, 2H), 1.59 (s, 6H), 1.14 (d, J=6.8 Hz, 12H).

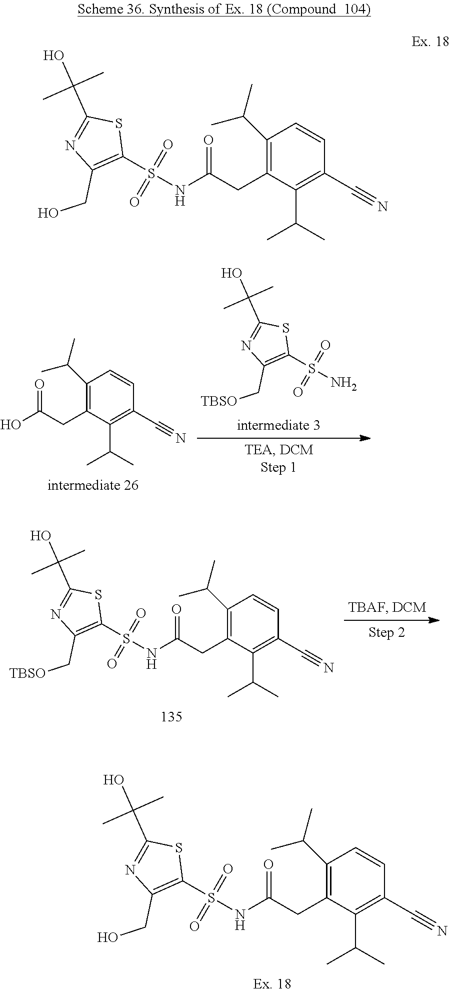 [SMILES: CC(C)c1ccc(C#N)c(C(C)C)c1CC(=O)NS(=O)(=O)c1sc(C(C)(C)O)nc1CO ; CC(C)c1ccc(C#N)c(C(C)C)c1CC(=O)O ; CC(C)(O)c1nc(CO[Si](C)(C)C(C)(C)C)c(S(N)(=O)=O)s1 ; CC(C)c1ccc(C#N)c(C(C)C)c1CC(=O)NS(=O)(=O)c1sc(C(C)(C)O)nc1CO[Si](C)(C)C(C)(C)C ; CC(C)c1ccc(C#N)c(C(C)C)c1CC(=O)NS(=O)(=O)c1sc(C(C)(C)O)nc1CO]

### 1. Synthesis of N-[(4-[[(tert-butyldimethylsilyl)oxy]methyl]-2-(2-hydroxypropan-2-yl)-1,3-thiazol-5-yl)sulfonyl]-2-[3-cyano-2,6-bis(propan-2-yl)phenyl]acetamide

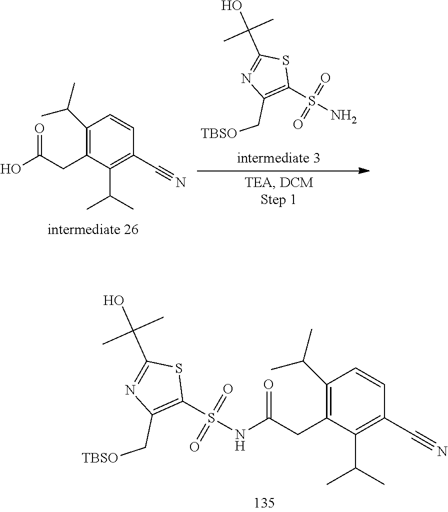 [SMILES: CC(C)c1ccc(C#N)c(C(C)C)c1CC(=O)O ; CC(C)(O)c1nc(CO[Si](C)(C)C(C)(C)C)c(S(N)(=O)=O)s1 ; CC(C)c1ccc(C#N)c(C(C)C)c1CC(=O)NS(=O)(=O)c1sc(C(C)(C)O)nc1CO[Si](C)(C)C(C)(C)C]

Into a 50-mL round-bottom flask purged with and maintained under an inert atmosphere of nitrogen, was placed 2-[3-cyano-2,6-bis(propan-2-yl)phenyl]acetic acid (100 mg, 0.408 mmol, 1 equiv), DCM (5 mL, 0.059 mmol, 0.14 equiv), (COCl)₂ (155.22 mg, 1.223 mmol, 3 equiv), and DMF (0.1 mL). The resulting solution was stirred for 1 hr at room temperature in a water bath, after which it was concentrated. This resulted in 100 mg (93.01%) of 2-[3-cyano-2,6-bis(propan-2-yl)phenyl]acetyl chloride as a yellow solid.

Into a 50-mL round-bottom flask purged and maintained with an inert atmosphere of nitrogen, was placed 4-[[(tert-butyldimethylsilyl)oxy]methyl]-2-(2-hydroxypropan-2-yl)-1,3-thiazole-5-sulfonamide (138.97 mg, 0.379 mmol, 1 equiv), DCM (5 mL, 78.650 mmol, 207.46 equiv), TEA (115.09 mg, 1.137 mmol, 3 equiv), and 2-[3-cyano-2,6-bis(propan-2-yl)phenyl]acetyl chloride (100 mg, 0.379 mmol, 1 equiv). The resulting solution was stirred for 1 hr at 0 degrees C. in a water/ice bath. The reaction was then quenched by the addition of 0.1 mL of water. The resulting mixture was concentrated. This resulted in 90 mg (39.97%) of N-[(4-[[(tert-butyldimethylsilyl)oxy]methyl]-2-(2-hydroxypropan-2-yl)-1,3-thiazol-5-yl)sulfonyl]-2-[3-cyano-2,6-bis(propan-2-yl)phenyl]acetamide as a yellow solid.

LCMS of N-[(4-[[(tert-butyldimethylsilyl)oxy]methyl]-2-(2-hydroxypropan-2-yl)-1,3-thiazol-5-yl)sulfonyl]-2-[3-cyano-2,6-bis(propan-2-yl)phenyl]acetamide: 594.4 [M+H]⁺, retention time 1.084 min. Method: Omega, 30*2.1 mm, 3.0 um column, 0.7 uL injection, 1.2 mL/min flow rate, 90-900 amu scan range, 190-400 nm UV range, Mobile phase A: Water (0.09% FA) and Mobile Phase B: MeCN(0.1% FA). 5% MPB to 95% in 0.9 min, hold at 95% MPB for 0.5 min, 95% MPB to 5% in 0.03 min.

### 2. Synthesis of 2-[3-cyano-2,6-bis(propan-2-yl)phenyl]-N-[[4-(hydroxymethyl)-2-(2-hydroxypropan-2-yl)-1,3-thiazol-5-yl]sulfonyl]acetamide

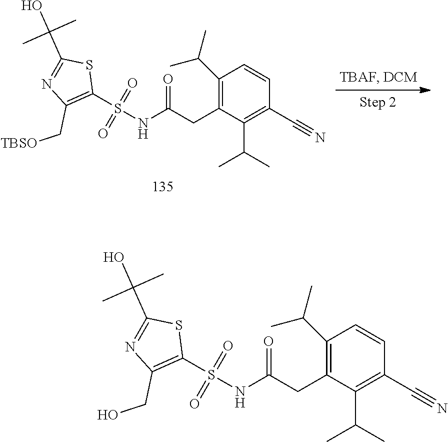 [SMILES: CC(C)c1ccc(C#N)c(C(C)C)c1CC(=O)NS(=O)(=O)c1sc(C(C)(C)O)nc1CO[Si](C)(C)C(C)(C)C ; CC(C)c1ccc(C#N)c(C(C)C)c1CC(=O)NS(=O)(=O)c1sc(C(C)(C)O)nc1CO]

Into a 50-mL round-bottom flask purged and maintained with an inert atmosphere of nitrogen, was placed N-[(4-[[(tert-butyldimethylsilyl)oxy]methyl]-2-(2-hydroxypropan-2-yl)-1,3-thiazol-5-yl)sulfonyl]-2-[3-cyano-2,6-bis(propan-2-yl)phenyl]acetamide (90 mg, 0.152 mmol, 1 equiv), THE (0.1 mL, 1.234 mmol, 8.14 equiv), TBAF (10 mL, 1.0 M in THF). The resulting solution was stirred for 1 hr at room temperature in a water bath. The resulting mixture was concentrated. The solids were filtered out. The crude product (200 mg) was purified by Prep-HPLC with the following conditions (Prep-HPLC-018): Column, XBridge Shield RP18 OBD Column, 19*250 mm, 10 um; mobile phase, Water(10 MMOL/L NH₄HCO₃) and ACN (12% PhaseB up to 47% in 7 min); Detector, UV. product was obtained. This resulted in 30 mg (41.27%) of 2-[3-cyano-2,6-bis(propan-2-yl)phenyl]-N-[[4-(hydroxymethyl)-2-(2-hydroxypropan-2-yl)-1,3-thiazol-5-yl]sulfonyl]acetamide as a white solid.

LCMS of 2-[3-cyano-2,6-bis(propan-2-yl)phenyl]-N-[[4-(hydroxymethyl)-2-(2-hydroxypropan-2-yl)-1,3-thiazol-5-yl]sulfonyl]acetamide (Method J). 480.1 [M+H]⁺, retention time 0.766 min.

¹H NMR-2-[3-cyano-2,6-bis(propan-2-yl)phenyl]-N-[[4-(hydroxymethyl)-2-(2-hydroxypropan-2-yl)-1,3-thiazol-5-yl]sulfonyl]acetamide (300 MHz, DMSO-d6) δ 7.57 (d, J=8.1 Hz, 1H), 7.25 (d, J=8.2 Hz, 2H), 7.00 (d, J=48.9 Hz, 2H), 6.08 (s, 1H), 5.14 (s, 1H), 4.64 (s, 2H), 3.65 (s, 2H), 3.15-3.05 (m, 1H), 1.45 (s, 6H), 1.22 (d, J=7.1 Hz, 6H), 1.07 (d, J=6.8 Hz, 6H).

Compounds 106, 115, 119-139, and 141 can be prepared using procedures analogous or similar to any of the procedures described herein for the preparation of compounds 101-105, 107-114, 116-118, 140, and 142.

### Assay 1

The following protocols are suitable for testing the activity of the compounds disclosed herein.

Procedure 1: IL-1β Production in PMA-Differentiated THP-1 Cells Stimulated with Gramicidin.

THP-1 cells were purchased from the American Type Culture Collection and sub-cultured according to instructions from the supplier. Cells were cultured in complete RPMI 1640 (containing 10% heat inactivated FBS, penicillin (100 units/ml) and streptomycin (100 μg/ml)), and maintained in log phase prior to experimental setup. Prior to the experiment, compounds were dissolved in dimethyl sulfoxide (DMSO) to generate a 30 mM stock. The compound stock was first pre-diluted in DMSO to 3, 0.34, 0.042 and 0.0083 mM intermediate concentrations and subsequently spotted using Echo550 liquid handler into an empty 384-well assay plate to achieve desired final concentration (e.g. 100, 33, 11, 3.7, 1.2, 0.41, 0.14, 0.046, 0.015, 0.0051, 0.0017 μM). DMSO was backfilled in the plate to achieve a final DMSO assay concentration of 0.37%. The plate was then sealed and stored at room temperature until required.

THP-1 cells were treated with PMA (Phorbol 12-myristate 13-acetate) (20 ng/ml) for 16-18 hours. On the day of the experiment the media was removed and adherent cells were detached with trypsin for 5 minutes. Cells were then harvested, washed with complete RPMI 1640, spun down, and resuspended in RPMI 1640 (containing 2% heat inactivated FBS, penicillin (100 units/ml) and streptomycin (100 μg/ml). The cells were plated in the 384-well assay plate containing the spotted compounds at a density of 50,000 cells/well (final assay volume 50 μl). Cells were incubated with compounds for 1 hour and then stimulated with gramicidin (5 μM) (Enzo) for 2 hours. Plates were then centrifuged at 340 g for 5 min. Cell free supernatant (40 μL) was collected using a 96-channel PlateMaster (Gilson) and the production of IL-1β was evaluated by HTRF (cisbio). The plates were incubated for 18 h at 4° C. and read using the preset HTRF program (donor emission at 620 nm, acceptor emission at 668 nm) of the SpectraMax i3x spectrophotometer (Molecular Devices, software SoftMax 6). A vehicle only control and a dose titration of CRID3 (100-0.0017 μM) were run concurrently with each experiment. Data was normalized to vehicle-treated samples (equivalent to 0% inhibition) and CRID3 at 100 μM (equivalent to 100% inhibition). Compounds exhibited a concentration-dependent inhibition of IL-1β production in PMA-differentiated THP-1 cells.

Procedure 2: IL-1β Production in PMA-Differentiated THP-1 Cells Stimulated with Gramicidin.

THP-1 cells were purchased from the American Type Culture Collection and sub-cultured according to instructions from the supplier. Prior to experiments, cells were cultured in complete RPMI 1640 (containing 10% heat inactivated FBS, penicillin (100 units/ml) and streptomycin (100 μg/ml)), and maintained in log phase prior to experimental setup. Prior to the experiment THP-1 were treated with PMA (Phorbol 12-myristate 13-acetate) (20 ng/ml) for 16-18 hours. Compounds were dissolved in dimethyl sulfoxide (DMSO) to generate a 30 mM stock. On the day of the experiment the media was removed and adherent cells were detached with trypsin for 5 minutes. Cells were then harvested, washed with complete RPMI 1640, spun down, resuspended in RPMI 1640 (containing 2% heat inactivated FBS, penicillin (100 units/ml) and streptomycin (100 μg/ml). The cells were plated in a 384-well plate at a density of 50,000 cells/well (final assay volume 50 μl). Compounds were first dissolved in assay medium to obtain a 5× top concentration of 500 μM. 10 step dilutions (1:3) were then undertaken in assay medium containing 1.67% DMSO. 5× compound solutions were added to the culture medium to achieve desired final concentration (e.g. 100, 33, 11, 3.7, 1.2, 0.41, 0.14, 0.046, 0.015, 0.0051, 0.0017 μM). Final DMSO concentration was at 0.37%. Cells were incubated with compounds for 1 hour and then stimulated with gramicidin (5 μM) (Enzo) for 2 hours. Plates were then centrifuged at 340 g for 5 min. Cell free supernatant (40 μL) was collected using a 96-channel PlateMaster (Gilson) and the production of IL-1β was evaluated by HTRF (cisbio). A vehicle only control and a dose titration of CRID3 (100-0.0017 μM) were run concurrently with each experiment. Data was normalized to vehicle-treated samples (equivalent to 0% inhibition) and CRID3 at 100 μM (equivalent to 100% inhibition). Compounds exhibited a concentration-dependent inhibition of IL-1β production in PMA-differentiated THP-1 cells.

Table 14 shows the biological activity of compounds in hTHP-1 assay containing 2% fetal bovine serum: <0.008 μM=“++++++”; ≥0.008 and ≤0.04 μM=“+++++”; ≥0.04 and <0.2 μM=“++++”; ≥0.2 and <1 μM=“+++”; ≥1 and <5 μM=“++”; ≥5 and <30 μM=“+”.

TABLE 14
Average IC₅₀ of compounds in hTHP-1 assay

| | | hTHP-1 IC₅₀ |
|---|---|---|
| Compound | Ex. # | μM |
| 101 | 2 | ++ |
| 102 | 3 | ++ |
| 103 | 5 | ++ |
| 104 | 18 | + |
| 105 | 6 | ++ |
| 106 | | + |
| 107 | 7 | +++ |
| 108 | 8 | +++ |
| 109 | 9 | + |
| 110 | 10 | +++ |
| 111 | 12 | + |
| 112 | 13 | >100 |
| 114 | 11 | >100 |
| 115 | | |
| 116 | 14 | ++ |
| 117 | 15 | +++ |
| 118 | 17 | ++ |
| 119 | | +++ |
| 120 | | + |
| 121 | | ++ |
| 122 | | ++ |
| 123 | | + |
| 124 | | >100 |
| 125 | | ++ |
| 126 | | >100 |
| 127 | | +++ |
| 128 | | +++ |
| 129 | | ++ |
| 130 | | ++ |
| 131 | | ++ |
| 132 | | + |
| 133 | | ++ |
| 134 | | ++ |
| 135 | | +++ |
| 136 | | +++ |
| 137 | | + |
| 138 | | ++ |
| 139 | | |
| 140 | 1 | ++++ |
| 141 | | |
| 142 | 4 | +++++ |

### Assay 2: Colon Pharmacokinetics in Mice

The test compound was formulated in 0.5% methyl-cellulose in water and dosed via oral gavage at 30 mg/kg to Male C57BL/6 Mouse. At various time points (typically 15 min, 30 min, 1, 2, 4, 6 and 8 h) post dosing, blood samples were removed via cardiac 25 puncture and intact colons were excised from the rats. Blood samples were centrifuged at 1500×g for 15 min to collect plasma. At the terminal time point each individual animal is anaesthetized, abdominal cavity is opened and from 2 cm below the caecum a 4 cm sample of the colon is dissected, cut open on the longitudinal axis and the solid contents removed by flushing with 2 mL of physiological fluid. The colon was further washed by putting it in 5 mL of physiological saline and shaken for 1 minute. The colon was pet dry weighed and transferred in 2 mL tubes. Colon will be weighted and homogenized with water by tissue weight (g) to water volume (mL) at ratio 1:3 before analysis. The actual concentration is the detected value multiplied by the dilution factor. A colon to plasma ratio was determined as the ratio of the colon conc. to the plasma conc. in pg hr/g. at 8 h time point.

| | | Mouse TD |
|---|---|---|
| | | Colon/Plasma |
| Compound # | Ex. # | @ 8 h |
| 117 | 15 | 3378 |
| 119 | | 6715 |
| 121 | | 152 |
| 122 | | 1482 |
| 127 | | 197 |
| 128 | | 1185 |
| 140 | 1 | 558 |

### Assay 3: Determination of Absorption in Cannulated Rats

Oral bioavailability (F %), fraction absorbed (Fa %) and fraction escaping hepatic 25 clearance (Fh %) were determined in Sprague Dawley rats from the following two studies: (I) Pharmacokinetics in rats following an IV dose of test compound (i.e., the analog being tested): Following IV dosing, plasma samples were typically collected from 0-24 hr. Drug levels were determined using an LC-MS-MS method. The resulting drug levels were used to compute the IV pharmacokinetic parameters: AUC IV and Dose IV. Rats that have been cannulated in their portal vein (PV) and also in their jugular vein (JV) were dosed orally with test compound. Following oral dosing, plasma samples were typically collected from 0-6 hr from both the portal vein and the jugular vein. Drug levels were determined using an LC-MS-MS method. The resulting drug levels were used to compute the following pharmacokinetic parameters: AUC PO PV, AUC PO JV5 and Dose PO. Using data derived from the above studies, the oral bioavailability F %, and the quantities Fa % and Fh % were calculated from the following formulas:

5F %=(AUC PO JV/AUC IV)*(Dose IV/Dose PO)*100

Fa %=(AUC PO PV/AUC IV)*(Dose IV/Dose PO)*100

Fh %=AUC PO JV/AUC PO PV

where:
AUC PO JV=Area under the curve following oral dose and plasma collected from the jugular vein;
AUC PO PV=Area under the curve following oral dose and plasma collected from the portal vein;
AUC IV=Area under the curve following an intravenous dose;
Dose IV=Intravenous Dose in mg/kg; and
15 Dose PO=Oral Dose in mg/kg

We claim:

1. A compound of Formula AA 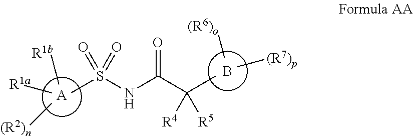
wherein
n=0 or 1;
o=1 or 2;
p=0, 1, 2, or 3;
wherein
A is a 5- to 10-membered heteroaryl or a C₆-C₁₀ aryl;
B is a 5- to 10-membered heteroaryl or a C₆-C₁₀ aryl;
wherein
R^{1a} is a C₁-C₆ alkyl, —CR¹¹R¹²NR¹¹R¹², or —SO₂NR¹¹R¹²;
wherein the C₁-C₆ alkyl is substituted with one or more hydroxy or —OSi(R¹³)₃;
R^{1b} is a C₁-C₆ alkyl substituted with one or more hydroxy, —SO₂NR¹¹R¹², —SO₂R¹³, —CONR¹¹R¹², —OR¹¹, —COR¹³; —CO₂R¹³, —NR¹³CONR¹¹R¹²; —CR¹¹R¹²CN, —NR¹¹SO₂R¹³, —NR¹¹CONR¹¹R¹², —CR¹¹R¹²NR¹¹R¹², CN, and —NR¹¹COR¹²;
at least one R⁶ is ortho to the bond connecting the B ring to the CR⁴R⁵ group of Formula AA;
R² is selected from C₁-C₆ alkyl, C₁-C₆ haloalkyl, C₁-C₆ alkoxy, C₁-C₆ haloalkoxy, halo, CN, NO₂, COC₁-C₆ alkyl, CO—C₆-C₁₀ aryl, CO(5- to 10-membered heteroaryl), CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, NHCOOC₁-C₆ alkyl, NH—(C═NR¹³)NR¹¹R¹², CONR⁸R⁹, SF₅, SC₁-C₆ alkyl, S(O₂)C₁-C₆ alkyl, S(O)C₁-C₆ alkyl, S(O₂)NR¹¹R¹², C₃-C₇ cycloalkyl and 3- to 7-membered heterocycloalkyl, wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), and OCO(3- to 7-membered heterocycloalkyl);
wherein each C₁-C₆ alkyl substituent and each C₁-C₆, alkoxy substituent of the R² C₃-C₇ cycloalkyl or of the R² 3- to 7-membered heterocycloalkyl is further optionally independently substituted with one to three hydroxy, halo, or oxo;
wherein the 3- to 7-membered heterocycloalkyl, C₆-C₁₀ aryl, 5- to 10-membered heteroaryl of the R² C₁-C₆ alkyl, the R² C₁-C₆ haloalkyl, the R² C₃-C₇ cycloalkyl, or the R² 3- to 7-membered heterocycloalkyl are optionally substituted with one or more substituents independently selected from halo, C₁-C₆ alkyl, and OC₁-C₆ alkyl;
R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₁₀ cycloalkyl and 3- to 10-membered heterocycloalkyl, and C₂-C₆ alkenyl,
wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, halo, C₆-C₁₀ aryl or NR⁸R⁹, or wherein
R⁶ or R⁷ is optionally fused to a five-to-seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;
each of R⁴ and R⁵ is independently selected from hydrogen and C₁-C₆ alkyl;
R¹⁰ is C₁-C₆ alkyl;
each of R⁸ and R⁹ at each occurrence is independently selected from hydrogen, C₁-C₆ alkyl, (C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or
R⁸ and R⁹ taken together with the nitrogen they are attached to form a 3- to 7-membered ring optionally containing one or more heteroatoms in addition to the nitrogen they are attached to;
R¹³ is C₁-C₆ alkyl, C₆-C₁₀ aryl, or 5- to 10-membered heteroaryl;
each of R¹¹ and R¹² at each occurrence is independently selected from hydrogen and C₁-C₆ alkyl optionally substituted with hydroxy;
with the proviso that the compound of Formula AA is not a compound selected from the group consisting of: 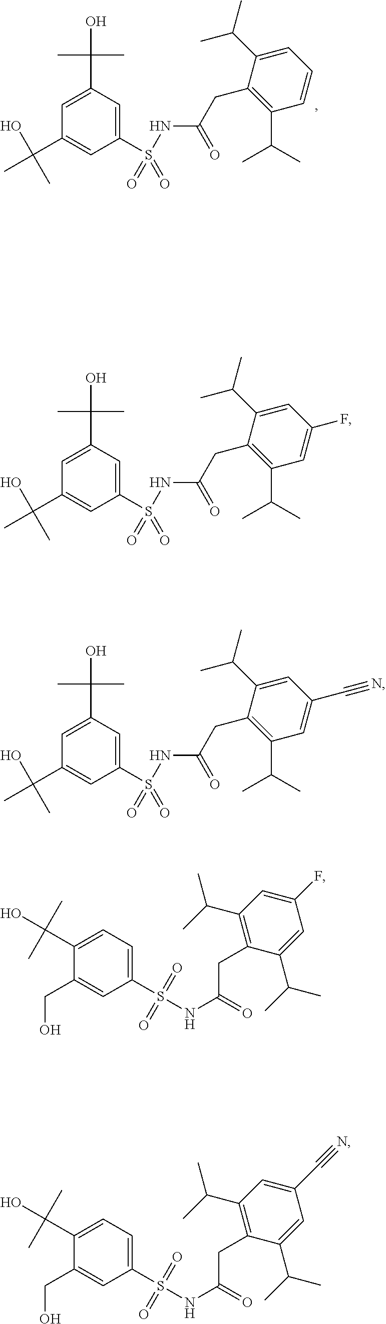 [SMILES: CC(C)c1cccc(C(C)C)c1CC(=O)NS(=O)(=O)c1cc(C(C)(C)O)cc(C(C)(C)O)c1 ; CC(C)c1cc(F)cc(C(C)C)c1CC(=O)NS(=O)(=O)c1cc(C(C)(C)O)cc(C(C)(C)O)c1 ; CC(C)c1cc(C#N)cc(C(C)C)c1CC(=O)NS(=O)(=O)c1cc(C(C)(C)O)cc(C(C)(C)O)c1 ; CC(C)c1cc(F)cc(C(C)C)c1CC(=O)NS(=O)(=O)c1ccc(C(C)(C)O)c(CO)c1 ; CC(C)c1cc(C#N)cc(C(C)C)c1CC(=O)NS(=O)(=O)c1ccc(C(C)(C)O)c(CO)c1] 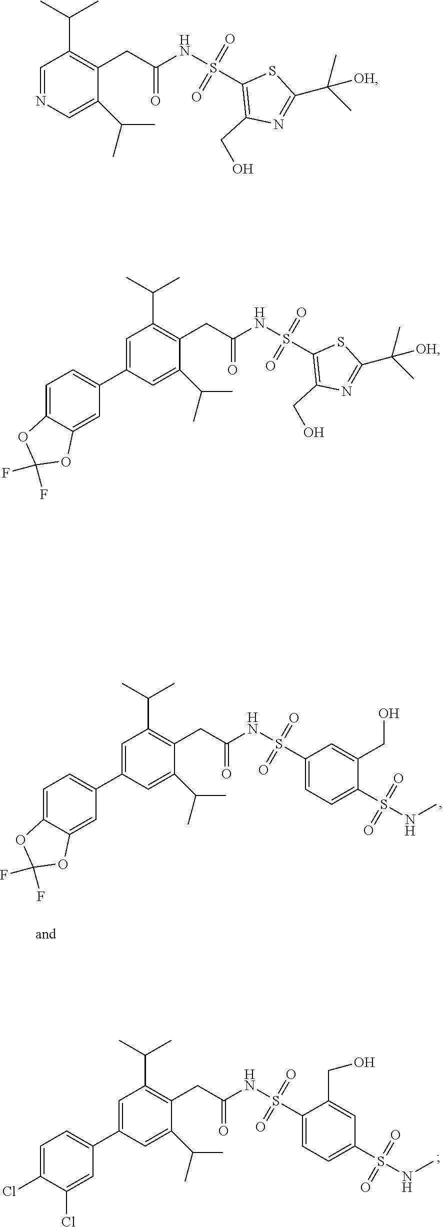 [SMILES: CC(C)c1cncc(C(C)C)c1CC(=O)NS(=O)(=O)c1sc(C(C)(C)O)nc1CO ; CC(C)c1cc(-c2ccc3c(c2)OC(F)(F)O3)cc(C(C)C)c1CC(=O)NS(=O)(=O)c1sc(C(C)(C)O)nc1CO ; CNS(=O)(=O)c1ccc(S(=O)(=O)NC(=O)Cc2c(C(C)C)cc(-c3ccc4c(c3)OC(F)(F)O4)cc2C(C)C)cc1CO ; CNS(=O)(=O)c1ccc(S(=O)(=O)NC(=O)Cc2c(C(C)C)cc(-c3ccc(Cl)c(Cl)c3)cc2C(C)C)c(CO)c1]
or a pharmaceutically acceptable salt thereof.

2. A compound of Formula AA 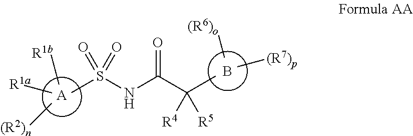
wherein the compound of Formula AA is selected from 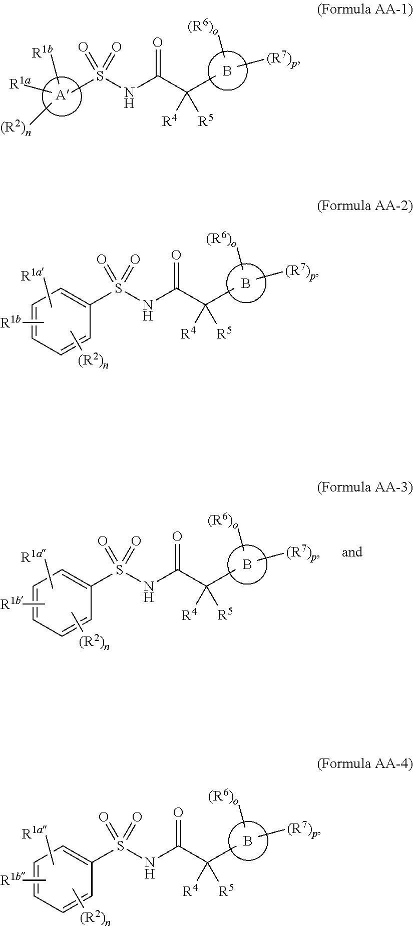
wherein
n=0 or 1;
o=1 or 2;
p=0, 1, 2, or 3;
wherein
A′ is a 5- to 10-membered heteroaryl;
B is a 5- to 10-membered heteroaryl or a C₆-C₁₀ aryl;
wherein
R^{1a} is a C₁-C₆ alkyl, —CR¹¹R¹²NR¹¹R¹² or —SO₂NR¹¹R¹²;
wherein the C₁-C₆ alkyl is substituted with one or more hydroxy or —OSi(R¹³)₃;
R^{1a′} is a C₁-C₆ alkyl, —CR¹¹R¹²NR¹¹R¹² or —SO₂NR¹¹R¹²;
wherein the C₁-C₆ alkyl is substituted with one or more —OSi(R¹³)₃;
R^{1a″} is a C₁-C₆ alkyl;
wherein the C₁-C₆ alkyl is substituted with one or more hydroxy;
R^{1b} is a C₁-C₆ alkyl substituted with one or more hydroxy, —SO₂NR¹¹R¹², —SO₂R¹³, —CONR¹¹R¹², —OR¹¹, —COR¹³; —CO₂R¹³, —NR¹³CONR¹¹R¹²; —CR¹¹R¹²CN, —NR¹¹SO₂R¹³, —NR¹¹CONR¹¹R¹², —CR¹¹R¹²NR¹¹R¹², CN, and —NR¹¹COR¹²;
R^{1b′} is —SO₂NR¹¹R¹², —SO₂R¹³, —CONR¹¹R¹², —OR¹¹, —COR¹³; —CO₂R¹³, —NR¹³CONR¹¹R¹²; —CR¹¹R¹²CN, —NR¹¹SO₂R¹³, —NR¹¹CONR¹¹R¹², —CR¹¹R¹²NR¹¹R¹², —CN, and —NR¹¹COR¹²;
R^{1b″} is a C₁-C₆ alkyl;
wherein the C₁-C₆ alkyl is substituted with one or more hydroxy;
at least one R⁶ is ortho to the bond connecting the B ring to the CR⁴R⁵ group of Formula AA-through Formula AA-1, AA-2, and AA-3;
at least one R⁶ is ortho to the bond connecting the B ring to the CR⁴R⁵ group of Formula AA-4;
R² is selected from C₁-C₆ alkyl, C₁-C₆ haloalkyl, C₁-C₆ alkoxy, C₁-C₆ haloalkoxy, halo, CN, NO₂, COC₁-C₆ alkyl, CO—C₆-C₁₀ aryl, CO(5- to 10-membered heteroaryl), CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, NHCOOC₁-C₆ alkyl, NH—(C═NR¹³)NR¹¹R¹², CONR⁸R⁹, SF₅, SC₁-C₆ alkyl, S(O₂)C₁-C₆ alkyl, S(O)C₁-C₆ alkyl, S(O₂)NR¹¹R¹², C₃-C₇ cycloalkyl, and 3- to 7-membered heterocycloalkyl, wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), and OCO(3- to 7-membered heterocycloalkyl);
wherein each C₁-C₆ alkyl substituent and each C₁-C₆, alkoxy substituent of the R² C₃-C₇ cycloalkyl or of the R² 3- to 7-membered heterocycloalkyl is further optionally independently substituted with one to three hydroxy, halo, or oxo;
wherein the 3- to 7-membered heterocycloalkyl, C₆-C₁₀ aryl, 5- to 10-membered heteroaryl of the R² C₁-C₆ alkyl, the R² C₁-C₆ haloalkyl, the R² C₃-C₇ cycloalkyl, or the R² 3- to 7-membered heterocycloalkyl are optionally substituted with one or more substituents independently selected from halo, C₁-C₆ alkyl, and OC₁-C₆ alkyl;
R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₃-C₆ alkyl, C₃-C₁₀ cycloalkyl and 3- to 10-membered heterocycloalkyl, and C₂-C₆ alkenyl,
wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, O(C₃-C₁₀ cycloalkyl), and S(O₂)C₁-C₆ alkyl; and wherein the C₁-C₆ alkyl or C₁-C₆ alkoxy that R⁶ or R⁷ is substituted with is optionally substituted with one or more hydroxyl, halo, C₆-C₁₀ aryl or NR⁸R⁹, or wherein R⁶ or R⁷ is optionally fused to a five-to-seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;
R^{6′} and R^{7′} are each independently selected from C₁-C₆ alkyl, C₁-C₆ haloalkyl, C₁-C₆ alkoxy, C₁-C₆ haloalkoxy, Cl, Br, I, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₁₀ cycloalkyl and 3- to 10-membered heterocycloalkyl, and C₂-C₆ alkenyl,
wherein R^{6′} and R^{7′} are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, O(C₃-C₁₀ cycloalkyl), and S(O₂)C₁-C₆ alkyl; and wherein the C₁-C₆ alkyl or C₁-C₆ alkoxy that R^{6′} or R^{7′} is substituted with is optionally substituted with one or more hydroxyl, halo, C₆-C₁₀ aryl or NR⁸R⁹, or wherein R^{6′} or R^{7′} is optionally fused to a five-to-seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
or at least one pair of R^{6′} and R^{7′} on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;
each of R⁴ and R⁵ is independently selected from hydrogen and C₁-C₆ alkyl;
R¹⁰ is C₁-C₆ alkyl;
each of R⁸ and R⁹ at each occurrence is independently selected from hydrogen, C₁-C₆ alkyl, (C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or
R⁸ and R⁹ taken together with the nitrogen they are attached to form a 3- to 7-membered ring optionally containing one or more heteroatoms in addition to the nitrogen they are attached to;
R¹³ is C₁-C₆ alkyl, C₆-C₁₀ aryl, or 5- to 10-membered heteroaryl;
each of R¹¹ and R¹² at each occurrence is independently selected from hydrogen and C₁-C₆ alkyl optionally substituted with hydroxy;
with the proviso that the compound of Formula AA is not a compound selected from the group consisting of: 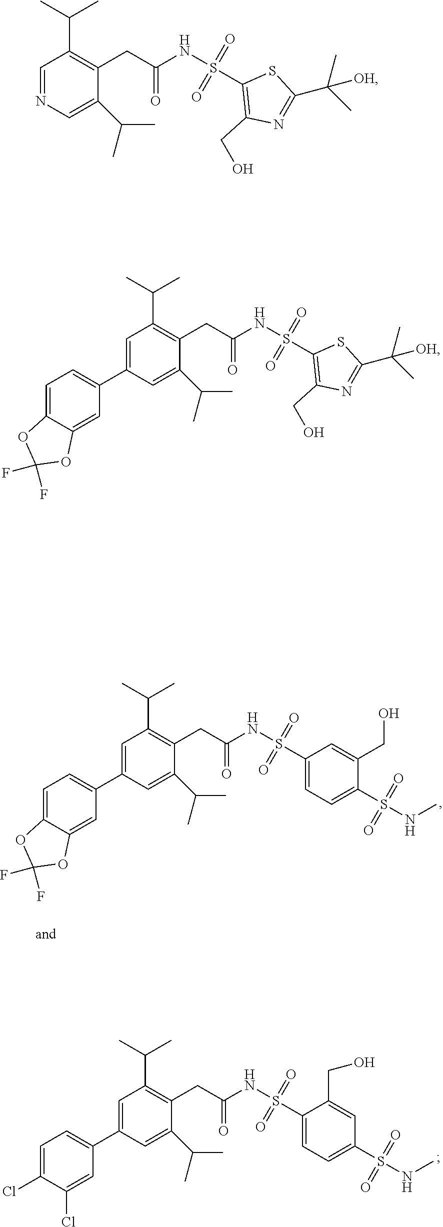 [SMILES: CC(C)c1cncc(C(C)C)c1CC(=O)NS(=O)(=O)c1sc(C(C)(C)O)nc1CO ; CC(C)c1cc(-c2ccc3c(c2)OC(F)(F)O3)cc(C(C)C)c1CC(=O)NS(=O)(=O)c1sc(C(C)(C)O)nc1CO ; CNS(=O)(=O)c1ccc(S(=O)(=O)NC(=O)Cc2c(C(C)C)cc(-c3ccc4c(c3)OC(F)(F)O4)cc2C(C)C)cc1CO ; CNS(=O)(=O)c1ccc(S(=O)(=O)NC(=O)Cc2c(C(C)C)cc(-c3ccc(Cl)c(Cl)c3)cc2C(C)C)c(CO)c1]
or a pharmaceutically acceptable salt thereof.

3. The compound of claim 2, wherein the compound of Formula AA is

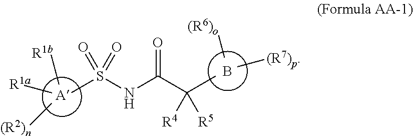

4. The compound of claim 2, wherein the compound of Formula AA is

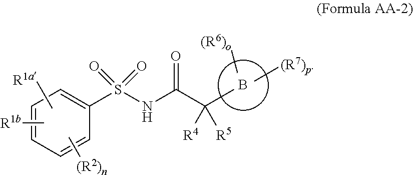

5. The compound of claim 2, wherein the compound of Formula AA is

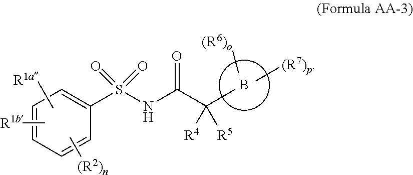

6. The compound of claim 2, wherein the compound of Formula AA is

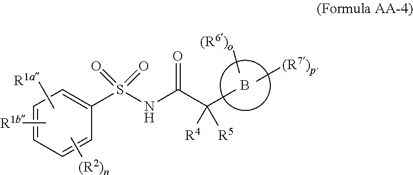

7. The compound of any one of claims 1-6, wherein each of R⁴ and R⁵ is hydrogen.

8. The compound of any one of claims 1-6, wherein one of R⁴ and R⁵ is C₁-C₆ alkyl.

9. The compound of any one of claims 1-3, 7, and 8, wherein A is a 5- to 6-membered heteroaryl containing 1 sulfur ring member.

10. The compound of any one of claims 1-3 and 7-9, wherein A is thiazolyl.

11. The compound of any one of claims 1-10, wherein n=0.

12. The compound of any one of claims 1, 2, 7-9, and 11, wherein the substituted ring A is selected from

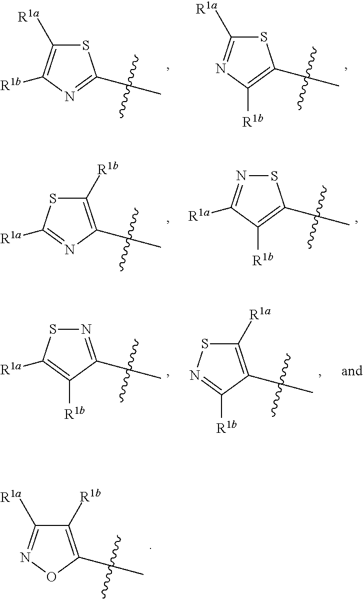

13. The compound of any one of claims 1, 2, 7-8, and 11, wherein the substituted ring A is

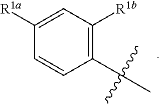

14. The compound of any one of claims 1-11, wherein n=1.

15. The compound of any one of claims 1, 2, 4-8, and 14, wherein the substituted ring A is selected from

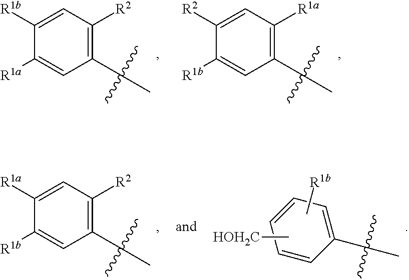

16. The compound of any one of claims 1, 2, 4, and 11, wherein the substituted ring A is selected from

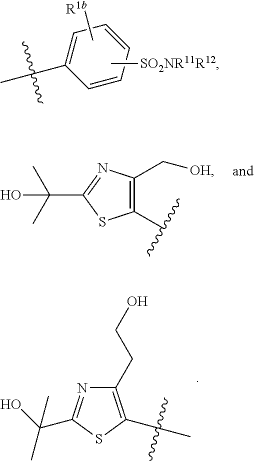

17. The compound of any one of claims 1, 2, 4, 7-8, and 11, wherein the substituted ring A is selected from

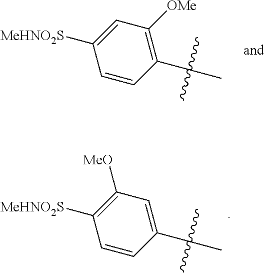

18. The compound of any one of claims 1-3 and 5-15, wherein R^{1a} is C₁-C₆ alkyl substituted with one or more hydroxy; or R^{1a} is C₁-C₆ alkyl substituted with one or more —OSi(R¹³)₃; or R^{1a} is —CR¹¹R¹²NR¹¹R¹²; or R^{1a} is —SO₂NR¹¹R¹².

19. The compound of any one of claims 2 and 4, wherein R^{1a′} is C₁-C₆ alkyl substituted with one or more —OSi(R¹³)₃; or R^{1a} is —CR¹¹R¹²NR¹¹R¹²; or
R^{1a′} is —SO₂NR¹¹R¹².

20. The compound of any one of claims 2 and 4, wherein R^{1b} is independently selected from the group consisting of C₁-C₆ alkyl substituted with one or more hydroxy, —SO₂NR¹¹R¹², —SO₂R¹³, —CONR¹¹R¹², —OR¹¹, —COR¹³; —NR¹³CONR¹¹R¹²; —CR¹¹R¹²CN, —NR¹¹SO₂R¹³, —NR¹¹CONR¹¹R¹², and —NR¹¹COR¹².

21. The compound of any one of claims 1-4 and 7-33, wherein R^{1b} is independently selected from the group consisting of —SO₂NR¹¹R¹², —SO₂R¹³, —CONR¹¹R¹², —COR¹³, —CO₂R¹³, —NR¹³CONR¹¹R¹²; and —CR¹¹R¹²CN.

22. The compound of any one of claims 1-4 and 7-18, wherein R^{1b} is —SO₂NHMe, SO₂NHCH₂CH₂OH, SO₂Me, CONHMe, or OMe.

23. The compound of any one of claims 1-4 and 7-18, wherein R^{1b} is —SO₂NHMe or OMe.

24. The compound of any one of claims 1-11, 14-15, and 18-23, wherein R² is independently selected from the group consisting of hydroxymethyl, C₂ alkyl substituted with hydroxy, C₃ alkyl substituted with hydroxy, C₄ alkyl substituted with hydroxy, C₅ alkyl substituted with hydroxy, and C₆ alkyl substituted with hydroxy; or
R² is selected from the group consisting of hydroxymethyl, 1-hydroxyethyl, 2-hydroxyethyl, 2-hydroxy-2-propyl, 3-hydroxy-2-propyl, 1-hydroxy-1-propyl, 2-hydroxy-1-propyl, 3-hydroxy-1-propyl, 4-hydroxy-1-butyl, 5-hydroxy-1-pentyl, and 6-hydroxy-1-hexyl; or
R² is selected from the group consisting of hydroxymethyl, 1-hydroxyethyl, 2-hydroxyethyl, 2-hydroxy-2-propyl, 3-hydroxy-2-propyl, 1-hydroxy-1-propyl, 2-hydroxy-1-propyl, 3-hydroxy-1-propyl, 4-hydroxy-1-butyl, and 6-hydroxy-1-hexyl; or
R² is selected from the group consisting of C₁-C₆ alkyl optionally substituted with one or more hydroxy, halo, oxo, or C₁-C₆ alkoxy; C₃-C₇ cycloalkyl optionally substituted with one or more hydroxy, halo, oxo, C₁-C₆ alkoxy, or C₁-C₆ alkyl wherein the C₁-C₆ alkoxy or C₁-C₆ alkyl is further optionally substituted with one to three hydroxy, halo, or oxo; 3- to 7-membered heterocycloalkyl optionally substituted with one or more hydroxy, halo, oxo, or C₁-C₆ alkyl, wherein the C₁-C₆ alkoxy or C₁-C₆ alkyl is further optionally substituted with one to three hydroxy, halo, or oxo; C₁-C₆, haloalkyl; C₁-C₆ alkoxy; C₁-C₆ haloalkoxy; halo; CN; CO—C₁-C₆ alkyl; CO—C₆-C₁₀ aryl; CO(5- to 10-membered heteroaryl); CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; S(O₂)NR¹¹R¹²; S(O)C₁-C₆ alkyl; and S(O₂)C₃-C₆ alkyl; or
R² is selected from the group consisting of fluoro, chloro, cyano, methyl, methoxy, ethoxy, isopropyl, l-hydroxy-2-methylpropan-2-yl, 2-hydroxy-2-propyl, hydroxymethyl, 1-hydroxy ethyl, 2-hydroxy ethyl, 1-hydroxy-2-propyl, 1-hydroxy-1-cyclopropyl, COCH₃, COPh, 2-methoxy-2-propyl, phenyl, S(O₂)CH₃, and S(O₂)NR¹¹R¹².

25. The compound of any one of claims 1-5, 7-24, wherein B is phenyl substituted with 1 or 2 R⁶ and optionally substituted with 1, 2, or 3 R⁷.

26. The compound of claim 25, wherein o=2 and p=0.

27. The compound of any one of claims 25 and 26, wherein the optionally substituted ring B is

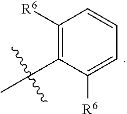

28. The compound of claim 27, wherein each R⁶ is independently selected from the group consisting of: C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl;
CONR⁸R⁹, and 4- to 6-membered heterocycloalkyl, wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl; or
wherein each R⁶ is independently selected from the group consisting of: C₁-C₆ alkyl, C₃-C₇ cycloalkyl, C₁-C₆ haloalkyl, C₁-C₆ alkoxy, and C₁-C₆ haloalkoxy, wherein the C₁-C₆ alkyl, C₁-C₆ haloalkyl, and C₃-C₇ cycloalkyl is optionally substituted with one or more substituents each independently selected from hydroxy, halo, CN, or oxo.

29. The compound of claim 25, wherein o=1 and p=1; or wherein o=2 and p=1.

30. The compound of claim 29, wherein the optionally substituted ring B is R⁶

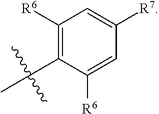

31. The compound of claim 30, wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy.

32. The compound of claim 25, wherein o=2 and p=2.

33. The compound of any one of claims 1-6, wherein the optionally substituted ring B is

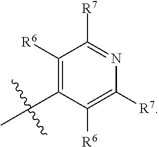

34. The compound of any one of claims 32 and 33, wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
or at least one pair of R⁶ and R⁷ on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

35. The compound of claim 32, wherein the optionally substituted ring B is

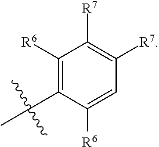

36. The compound of claim 35, wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
or R⁶ and R⁷, taken together with the atoms connecting them, independently form C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

37. The compound of claim 25, wherein o=2 and p=3

38. The compound of claim 37, wherein the optionally substituted ring B is

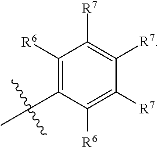

39. The compound of claim 38, wherein the optionally substituted ring B is

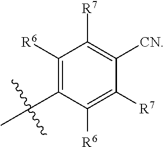

40. The compound of any one of claims 1-6, wherein the optionally substituted ring B is

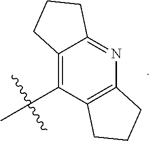 [SMILES: *c1c2c(nc3c1CCC3)CCC2]

41. The compound of any one of claims 38 and 39, wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
or at least one pair of R⁶ and R⁷ on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

42. The compound of any one of claims 1-6, 7-25, 32-33, 35, and 37-39, wherein two pairs of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

43. The compound of any one of claims 1-5, 7-27, 29-30, 32, 33, 35, and 37-39, wherein each R⁶ is independently selected from CN, C₁-C₆ alkyl, 5- to 10-membered heteroaryl, and 3- to 7-membered heterocycloalkyl;
wherein the C₁-C₆ alkyl is optionally substituted with one or more substituents each independently selected from hydroxyl or C₁-C₆ alkoxy.

44. The compound of any one of claims 1-5, 7-26, 29-30, 32, 33, 35, and 38-40, wherein each R⁷ is independently selected from CN, C₁-C₆ alkyl, 5- to 10-membered heteroaryl, and 3- to 7-membered heterocycloalkyl;
wherein the C₁-C₆ alkyl is optionally substituted with one or more substituents each independently selected from hydroxyl or C₁-C₆ alkoxy.

45. The compound of any one of the preceding claims, wherein R³ is hydrogen.

46. A compound selected from the group consisting of the compounds below:

| Comp | | |
|---|---|---|
| ound | Structure | m/z |
| 101 | 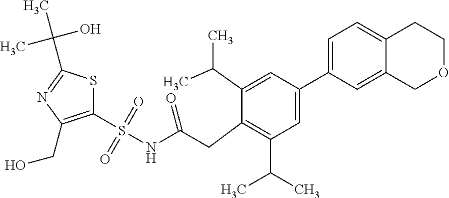 [SMILES: CC(C)c1cc(-c2ccc3c(c2)COCC3)cc(C(C)C)c1CC(=O)NS(=O)(=O)c1sc(C(C)(C)O)nc1CO] | 587.2 |
| 102 | 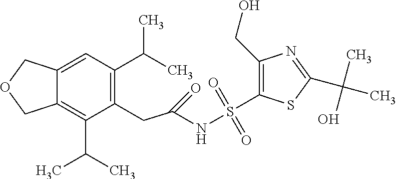 [SMILES: CC(C)c1cc2c(c(C(C)C)c1CC(=O)NS(=O)(=O)c1sc(C(C)(C)O)nc1CO)COC2] | 497.2 |
| 103 | 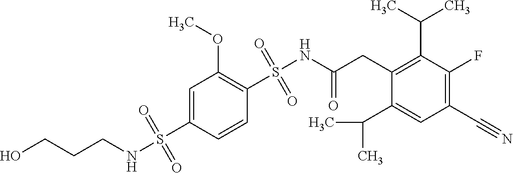 [SMILES: COc1cc(S(=O)(=O)NCCCO)ccc1S(=O)(=O)NC(=O)Cc1c(C(C)C)cc(C#N)c(F)c1C(C)C] | 570 |
| 104 | 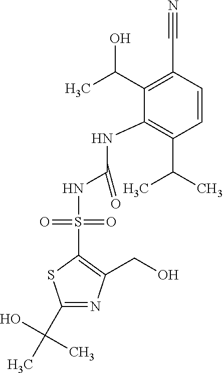 [SMILES: CC(C)c1ccc(C#N)c(C(C)O)c1NC(=O)NS(=O)(=O)c1sc(C(C)(C)O)nc1CO] | 480.1 |
| 105 | 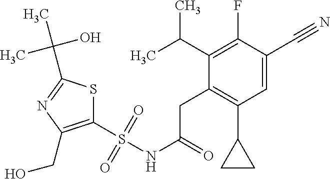 [SMILES: CC(C)c1c(F)c(C#N)cc(C2CC2)c1CC(=O)NS(=O)(=O)c1sc(C(C)(C)O)nc1CO] | 496.1 |
| 106 | 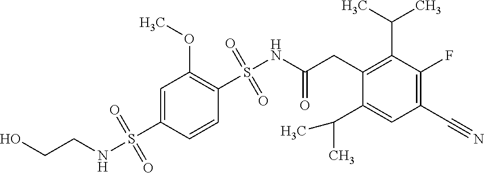 [SMILES: COc1cc(S(=O)(=O)NCCO)ccc1S(=O)(=O)NC(=O)Cc1c(C(C)C)cc(C#N)c(F)c1C(C)C] | 555.1 |
| 107 | 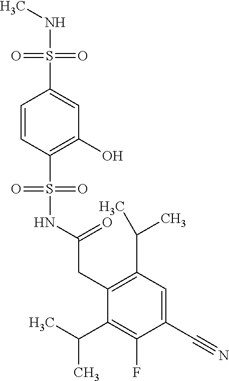 [SMILES: CNS(=O)(=O)c1ccc(S(=O)(=O)NC(=O)Cc2c(C(C)C)cc(C#N)c(F)c2C(C)C)c(O)c1] | 512.2 |
| 108 | 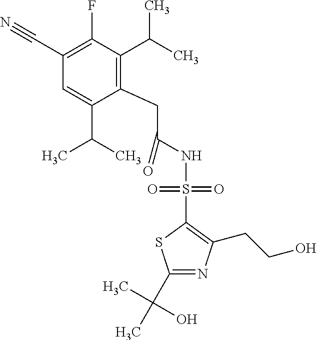 [SMILES: CC(C)c1cc(C#N)c(F)c(C(C)C)c1CC(=O)NS(=O)(=O)c1sc(C(C)(C)O)nc1CCO] | 512.3 |
| 109 | 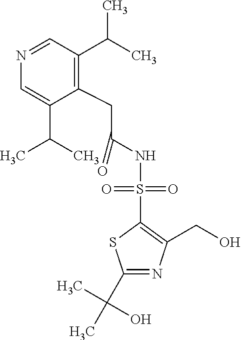 [SMILES: CC(C)c1cncc(C(C)C)c1CC(=O)NS(=O)(=O)c1sc(C(C)(C)O)nc1CO] | 455.15 |
| 110 | 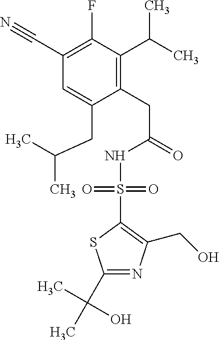 [SMILES: CC(C)Cc1cc(C#N)c(F)c(C(C)C)c1CC(=O)NS(=O)(=O)c1sc(C(C)(C)O)nc1CO] | 510.2 |
| 111 | 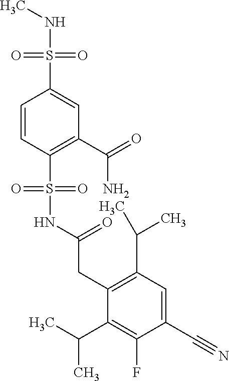 [SMILES: CNS(=O)(=O)c1ccc(S(=O)(=O)NC(=O)Cc2c(C(C)C)cc(C#N)c(F)c2C(C)C)c(C(N)=O)c1] | 538.1 |
| 112 | 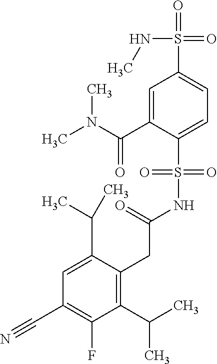 [SMILES: CNS(=O)(=O)c1ccc(S(=O)(=O)NC(=O)Cc2c(C(C)C)cc(C#N)c(F)c2C(C)C)c(C(=O)N(C)C)c1] | 567.3 |
| 114 | 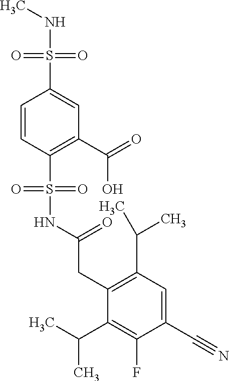 [SMILES: CNS(=O)(=O)c1ccc(S(=O)(=O)NC(=O)Cc2c(C(C)C)cc(C#N)c(F)c2C(C)C)c(C(=O)O)c1] | 538.1 |
| 115 | 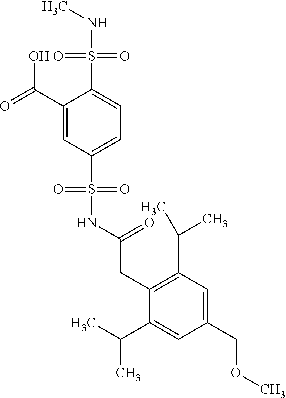 [SMILES: CNS(=O)(=O)c1ccc(S(=O)(=O)NC(=O)Cc2c(C(C)C)cc(COC)cc2C(C)C)cc1C(=O)O] | 540.1 |
| 116 | 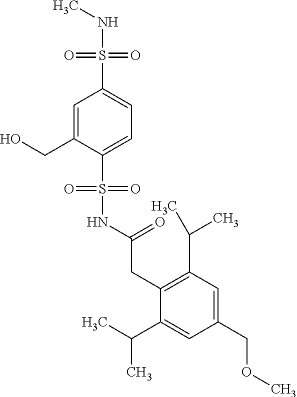 [SMILES: CNS(=O)(=O)c1ccc(S(=O)(=O)NC(=O)Cc2c(C(C)C)cc(COC)cc2C(C)C)c(CO)c1] | 526.1 |
| 117 | 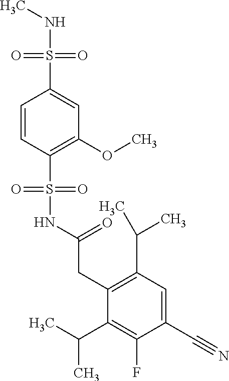 [SMILES: CNS(=O)(=O)c1ccc(S(=O)(=O)NC(=O)Cc2c(C(C)C)cc(C#N)c(F)c2C(C)C)c(OC)c1] | 524.0 |
| 118 | 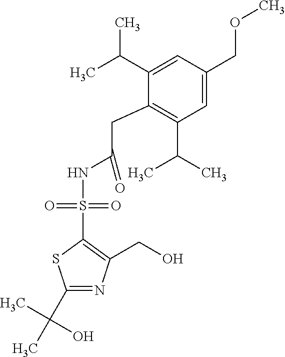 [SMILES: COCc1cc(C(C)C)c(CC(=O)NS(=O)(=O)c2sc(C(C)(C)O)nc2CO)c(C(C)C)c1] | 497.1 |
| 119 | 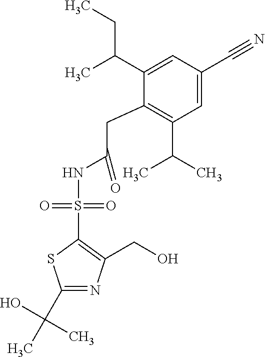 [SMILES: CCC(C)c1cc(C#N)cc(C(C)C)c1CC(=O)NS(=O)(=O)c1sc(C(C)(C)O)nc1CO] | 493.1 |
| 120 | 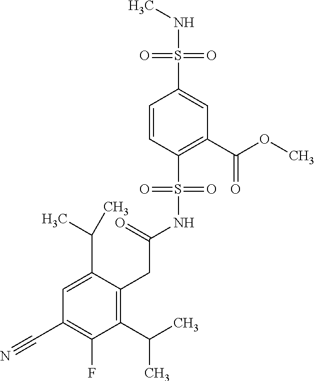 [SMILES: CNS(=O)(=O)c1ccc(S(=O)(=O)NC(=O)Cc2c(C(C)C)cc(C#N)c(F)c2C(C)C)c(C(=O)OC)c1] | 553.1 |
| 121 | 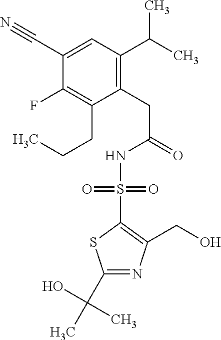 [SMILES: CCCc1c(F)c(C#N)cc(C(C)C)c1CC(=O)NS(=O)(=O)c1sc(C(C)(C)O)nc1CO] | 497.1 |
| 122 | 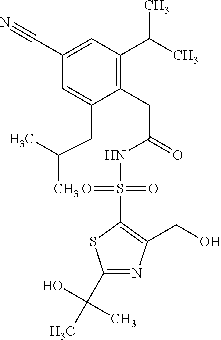 [SMILES: CC(C)Cc1cc(C#N)cc(C(C)C)c1CC(=O)NS(=O)(=O)c1sc(C(C)(C)O)nc1CO] | 493.6 |
| 123 | 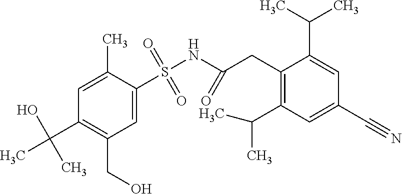 [SMILES: Cc1cc(C(C)(C)O)c(CO)cc1S(=O)(=O)NC(=O)Cc1c(C(C)C)cc(C#N)cc1C(C)C] | 486.6 |
| 124 | 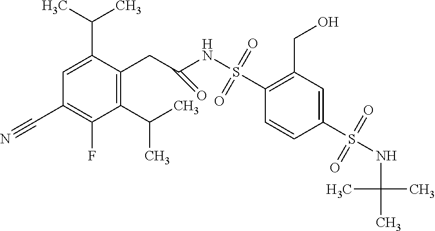 [SMILES: CC(C)c1cc(C#N)c(F)c(C(C)C)c1CC(=O)NS(=O)(=O)c1ccc(S(=O)(=O)NC(C)(C)C)cc1CO] | 567.1 |
| 125 | 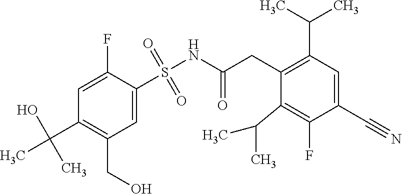 [SMILES: CC(C)c1cc(C#N)c(F)c(C(C)C)c1CC(=O)NS(=O)(=O)c1cc(CO)c(C(C)(C)O)cc1F] | 508.1 |
| 126 | 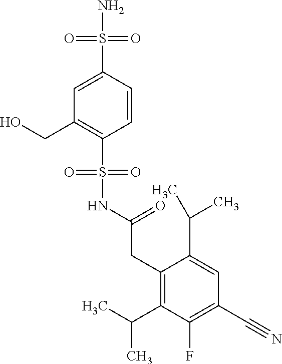 [SMILES: CC(C)c1cc(C#N)c(F)c(C(C)C)c1CC(=O)NS(=O)(=O)c1ccc(S(N)(=O)=O)cc1CO] | 511.1 |
| 127 | 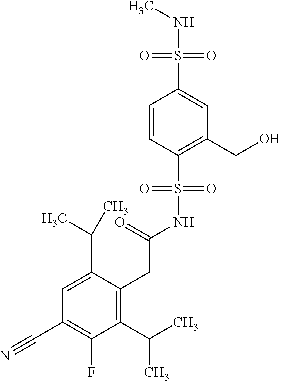 [SMILES: CNS(=O)(=O)c1ccc(S(=O)(=O)NC(=O)Cc2c(C(C)C)cc(C#N)c(F)c2C(C)C)c(CO)c1] | 525.1 |
| 128 | 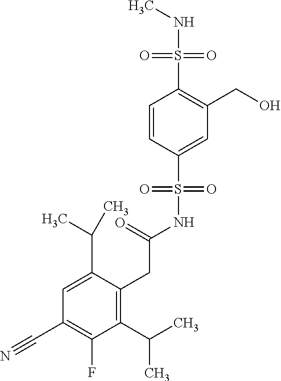 [SMILES: CNS(=O)(=O)c1ccc(S(=O)(=O)NC(=O)Cc2c(C(C)C)cc(C#N)c(F)c2C(C)C)cc1CO] | 525.1 |
| 129 | 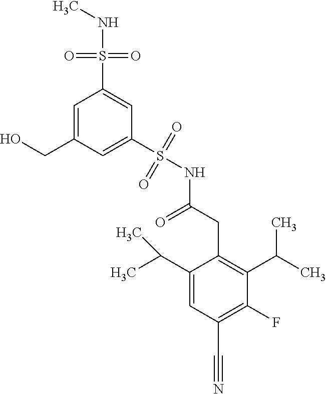 [SMILES: CNS(=O)(=O)c1cc(CO)cc(S(=O)(=O)NC(=O)Cc2c(C(C)C)cc(C#N)c(F)c2C(C)C)c1] | 525.1 |
| 130 | 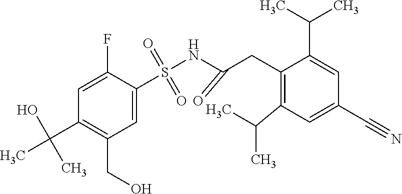 [SMILES: CC(C)c1cc(C#N)cc(C(C)C)c1CC(=O)NS(=O)(=O)c1cc(CO)c(C(C)(C)O)cc1F] | 490.1 |
| 131 | 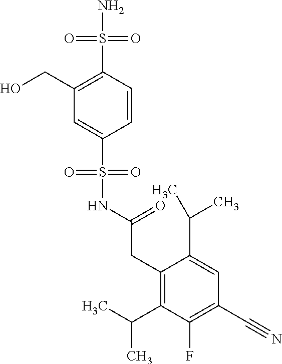 [SMILES: CC(C)c1cc(C#N)c(F)c(C(C)C)c1CC(=O)NS(=O)(=O)c1ccc(S(N)(=O)=O)c(CO)c1] | 511.1 |
| 132 | 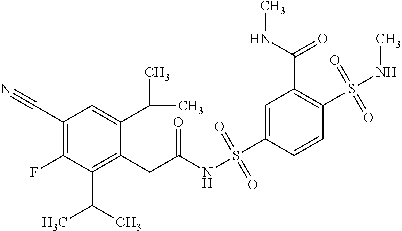 [SMILES: CNC(=O)c1cc(S(=O)(=O)NC(=O)Cc2c(C(C)C)cc(C#N)c(F)c2C(C)C)ccc1S(=O)(=O)NC] | 552.1 |
| 133 | 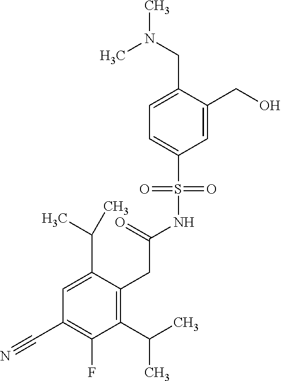 [SMILES: CC(C)c1cc(C#N)c(F)c(C(C)C)c1CC(=O)NS(=O)(=O)c1ccc(CN(C)C)c(CO)c1] | 489.1 |
| 134 | 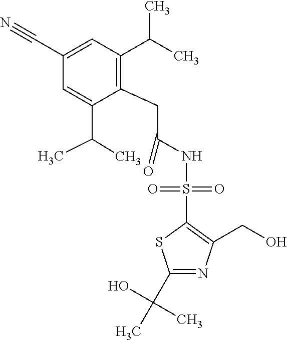 [SMILES: CC(C)c1cc(C#N)cc(C(C)C)c1CC(=O)NS(=O)(=O)c1sc(C(C)(C)O)nc1CO] | 497.1 |
| 135 | 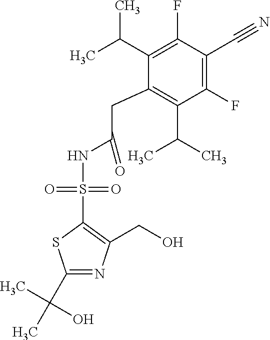 [SMILES: CC(C)c1c(F)c(C#N)c(F)c(C(C)C)c1CC(=O)NS(=O)(=O)c1sc(C(C)(C)O)nc1CO] | 515.1 |
| 136 | 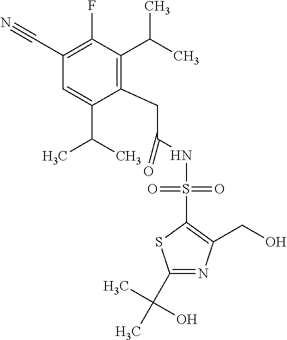 [SMILES: CC(C)c1cc(C#N)c(F)c(C(C)C)c1CC(=O)NS(=O)(=O)c1sc(C(C)(C)O)nc1CO] | 497.1 |
| 137 | 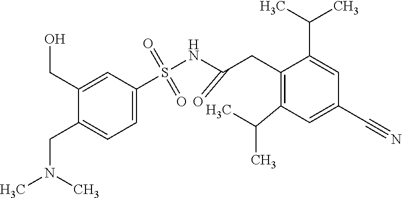 [SMILES: CC(C)c1cc(C#N)cc(C(C)C)c1CC(=O)NS(=O)(=O)c1ccc(CN(C)C)c(CO)c1] | 471.1 |
| 138 | 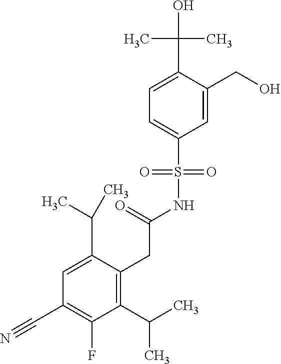 [SMILES: CC(C)c1cc(C#N)c(F)c(C(C)C)c1CC(=O)NS(=O)(=O)c1ccc(C(C)(C)O)c(CO)c1] | 490.1 |
| 139 | 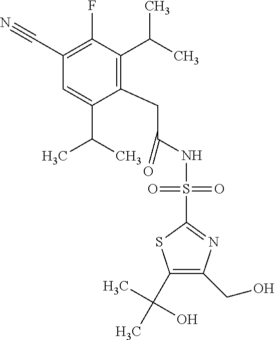 [SMILES: CC(C)c1cc(C#N)c(F)c(C(C)C)c1CC(=O)NS(=O)(=O)c1nc(CO)c(C(C)(C)O)s1] | 497.1 |
| 140 | 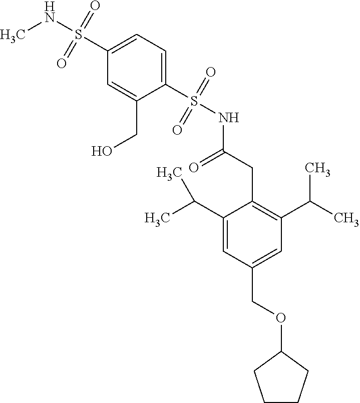 [SMILES: CNS(=O)(=O)c1ccc(S(=O)(=O)NC(=O)Cc2c(C(C)C)cc(COC3CCCC3)cc2C(C)C)c(CO)c1] | 581.3 |
| 141 | 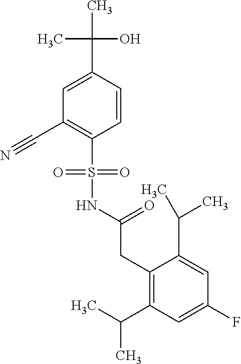 [SMILES: CC(C)c1cc(F)cc(C(C)C)c1CC(=O)NS(=O)(=O)c1ccc(C(C)(C)O)cc1C#N] | 460.1 |
| 142 | 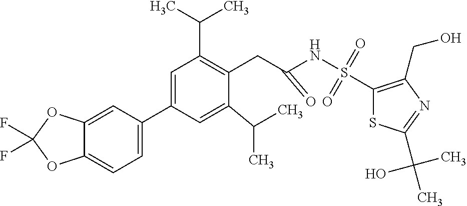 [SMILES: CC(C)c1cc(-c2ccc3c(c2)OC(F)(F)O3)cc(C(C)C)c1CC(=O)NS(=O)(=O)c1sc(C(C)(C)O)nc1CO] | 610.1 |

and pharmaceutically acceptable salts thereof.

47. A pharmaceutical composition comprising a compound or salt as claimed in any one of claims 1-46 and one or more pharmaceutically acceptable excipients.

48. A method for modulating NRLP3 activity, the method comprising contacting NRLP3 with an effective amount of a compound as claimed in any one of claims 1-46 or a pharmaceutical composition as claimed in claim 47.

49. The method of claim 48, wherein the modulating comprises antagonizing NRLP3.

50. A method of treating a disease, disorder or condition that is a metabolic disorder, comprising administering to a subject in need of such treatment an effective amount of a compound as claimed in any one of claims 1-46 or a pharmaceutical composition as claimed in claim 47.

51. The method of claim 50, wherein the metabolic disorder is Type 2 diabetes, atherosclerosis, obesity or gout.

52. A method of treating a disease, disorder or condition that is a disease of the central nervous system, comprising administering to a subject in need of such treatment an effective amount of a compound as claimed in any one of claims 1-46 or a pharmaceutical composition as claimed in claim 47.

53. The method of claim 52, wherein the disease of the central nervous system is Alzheimer's disease, multiple sclerosis, Amyotrophic Lateral Sclerosis or Parkinson's disease.

54. A method of treating a disease, disorder or condition that is lung disease, comprising administering to a subject in need of such treatment an effective amount of a compound as claimed in any one of claims 1-46 or a pharmaceutical composition as claimed in claim 47.

55. The method of claim 54, wherein the lung disease is asthma, COPD or pulmonary idiopathic fibrosis.

56. A method of treating a disease, disorder or condition that is liver disease, comprising administering to a subject in need of such treatment an effective amount of a compound as claimed in any one of claims 1-46 or a pharmaceutical composition as claimed in claim 47.

57. The method of claim 56, wherein the liver disease is NASH syndrome, viral hepatitis or cirrhosis.

58. A method of treating a disease, disorder or condition that is pancreatic disease, comprising administering to a subject in need of such treatment an effective amount of a compound as claimed in any one of claims 1-46 or a pharmaceutical composition as claimed in claim 47.

59. The method of claim 58, wherein the pancreatic disease is acute pancreatitis or chronic pancreatitis.

60. A method of treating a disease, disorder or condition that is kidney disease, comprising administering to a subject in need of such treatment an effective amount of a compound as claimed in any one of claims 1-46 or a pharmaceutical composition as claimed in claim 47.

61. The method of claim 60, wherein the kidney disease is acute kidney injury or chronic kidney injury.

62. A method of treating a disease, disorder or condition that is intestinal disease, comprising administering to a subject in need of such treatment an effective amount of a compound as claimed in any one of claims 1-46 or a pharmaceutical composition as claimed in claim 47.

63. The method of claim 62, wherein the intestinal disease is Crohn's disease or Ulcerative Colitis.

64. A method of treating a disease, disorder or condition that is skin disease, comprising administering to a subject in need of such treatment an effective amount of a compound as claimed in any one of claims 1-46 or a pharmaceutical composition as claimed in claim 47.

65. The method of claim 64, wherein the skin disease is psoriasis.

66. A method of treating a disease, disorder or condition that is musculoskeletal disease, comprising administering to a subject in need of such treatment an effective amount of a compound as claimed in any one of claims 1-46 or a pharmaceutical composition as claimed in claim 47.

67. The method of claim 66, wherein the musculoskeletal disease is scleroderma.

68. A method of treating a disease, disorder or condition that is a vessel disorder, comprising administering to a subject in need of such treatment an effective amount of a compound as claimed in any one of claims 1-46 or a pharmaceutical composition as claimed in claim 47.

69. The method of claim 68, wherein the vessel disorder is giant cell arteritis.

70. A method of treating a disease, disorder or condition that is a disorder of the bones, comprising administering to a subject in need of such treatment an effective amount of a compound as claimed in any one of claims 1-46 or a pharmaceutical composition as claimed in claim 47.

71. The method of claim 70, wherein the disorder of the bones is osteoarthritis, osteoporosis or osteopetrosis disorders.

72. A method of treating a disease, disorder or condition that is eye disease, comprising administering to a subject in need of such treatment an effective amount of a compound as claimed in any one of claims 1-46 or a pharmaceutical composition as claimed in claim 47.

73. The method of claim 72, wherein the eye disease is glaucoma or macular degeneration.

74. A method of treating a disease, disorder or condition that is a disease caused by viral infection, comprising administering to a subject in need of such treatment an effective amount of a compound as claimed in any one of claims 1-46 or a pharmaceutical composition as claimed in claim 47.

75. The method of claim 74, wherein the diseases caused by viral infection is HIV or AIDS.

76. A method of treating a disease, disorder or condition that is an autoimmune disease, comprising administering to a subject in need of such treatment an effective amount of a compound as claimed in any one of claims 1-46 or a pharmaceutical composition as claimed in claim 47.

77. The method of claim 76, wherein the autoimmune disease is Rheumatoid Arthritis, Systemic Lupus Erythematosus, Autoimmune Thyroiditis.

78. A method of treating a disease, disorder or condition that is cancer or aging, comprising administering to a subject in need of such treatment an effective amount of a compound as claimed in any one of claims 1-46 or a pharmaceutical composition as claimed in claim 47.

79. A method of treating a disease, disorder or condition that is a cancer selected from: myelodysplastic syndromes (MDS); non-small cell lung cancer, such as non-small cell lung cancer in patients carrying mutation or overexpression of NLRP3; acute lymphoblastic leukemia (ALL), such as ALL in patients resistant to glucocorticoids treatment; Langerhan's cell histiocytosis (LCH); multiple myeloma; promyelocytic leukemia; acute myeloid leukemia (AML) chronic myeloid leukemia (CML); gastric cancer; and lung cancer metastasis, comprising administering to a subject in need of such treatment an effective amount of a compound as claimed in any one of claims 1-46 or a pharmaceutical composition as claimed in claim 47.

80. The method of any one of claims 49-79, further comprising administering a therapeutically effective amount of an anti-TNFα agent to the subject.


SEQUENCE LISTING (37 sequences)

SEQ ID NO: 1 - DNA, 702 bp, Homo sapiens
atgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtga

SEQ ID NO: 2 - DNA, 1368 bp, Homo sapiens
atgggcctctccaccgtgcctgacctgctgctgccactggtgctcctggagctgttggtgggaatatacccctcaggggttattggactggtccctcacctaggggacagggagaagagagatagtgtgtgtccccaaggaaaatatatccaccctcaaaataattcgatttgctgtaccaagtgccacaaaggaacctacttgtacaatgactgtccaggcccggggcaggatacggactgcagggagtgtgagagcggctccttcaccgcttcagaaaaccacctcagacactgcctcagctgctccaaatgccgaaaggaaatgggtcaggtggagatctcttcttgcacagtggaccgggacaccgtgtgtggctgcaggaagaaccagtaccggcattattggagtgaaaaccttttccagtgcttcaattgcagcctctgcctcaatgggaccgtgcacctctcctgccaggagaaacagaacaccgtgtgcacctgccatgcaggtttctttctaagagaaaacgagtgtgtctcctgtagtaactgtaagaaaagcctggagtgcacgaagttgtgcctaccccagattgagaatgttaagggcactgaggactcaggcaccacagtgctgttgcccctggtcattttctttggtctttgccttttatccctcctcttcattggtttaatgtatcgctaccaacggtggaagtccaagctctactccattgtttgtgggaaatcgacacctgaaaaagagggggagcttgaaggaactactactaagcccctggccccaaacccaagcttcagtcccactccaggcttcacccccaccctgggcttcagtcccgtgcccagttccaccttcacctccagctccacctatacccccggtgactgtcccaactttgcggctccccgcagagaggtggcaccaccctatcagggggctgaccccatccttgcgacagccctcgcctccgaccccatccccaacccccttcagaagtgggaggacagcgcccacaagccacagagcctagacactgatgaccccgcgacgctgtacgccgtggtggagaacgtgcccccgttgcgctggaaggaattcgtgcggcgcctagggctgagcgaccacgagatcgatcggctggagctgcagaacgggcgctgcctgcgcgaggcgcaatacagcatgctggcgacctggaggcggcgcacgccgcggcgcgaggccacgctggagctgctgggacgcgtgctccgcgacatggacctgctgggctgcctggaggacatcgaggaggcgctttgcggccccgccgccctcccgcccgcgcccagtcttctcagatga

SEQ ID NO: 3 - DNA, 281 bp, Homo sapiens
attcttcccctggtggccatgggacccaggtcaatgtcacctgcatcgtgaacgtctgtagcagctctgaccacagctcacagtgctcctcccaagccagctccacaatgggagacacagattccagcccctcggagtccccgaaggacgagcaggtccccttctccaaggaggaatgtgcctttcggtcacagctggagacgccagagaccctgctggggagcaccgaagagaagcccctgccccttggagtgcctgatgctgggatgaagcccagttaa

SEQ ID NO: 4 - DNA, 939 bp, Homo sapiens
atggcagctgggcaaaatgggcacgaagagtgggtgggcagcgcatacctgtttgtggagtcctcgctggacaaggtggtcctgtcggatgcctacgcgcacccccagcagaaggtggcagtgtacagggctctgcaggctgccttggcagagagcggcgggagcccggacgtgctgcagatgctgaagatccaccgcagcgacccgcagctgatcgtgcagctgcgattctgcgggcggcagccctgtggccgcttcctccgcgcctaccgcgagggggcgctgcgcgccgcgctgcagaggagcctggcggccgcgctcgcccagcactcggtgccgctgcaactggagctgcgcgccggcgccgagcggctggacgctttgctggcggacgaggagcgctgtttgagttgcatcctagcccagcagcccgaccggctccgggatgaagaactggctgagctggaggatgcgctgcgaaatctgaagtgcggctcgggggcccggggtggcgacggggaggtcgcttcggcccccttgcagcccccggtgccctctctgtcggaggtgaagccgccgccgccgccgccacctgcccagacttttctgttccagggtcagcctgtagtgaatcggccgctgagcctgaaggaccaacagacgttcgcgcgctctgtgggtctcaaatggcgcaaggtggggcgctcactgcagcgaggctgccgggcgctgcgggacccggcgctggactcgctggcctacgagtacgagcgcgagggactgtacgagcaggccttccagctgctgcggcgcttcgtgcaggccgagggccgccgcgccacgctgcagcgcctggtggaggcactcgaggagaacgagctcaccagcctggcagaggacttgctgggcctgaccgatcccaatggcggcctggcctag

SEQ ID NO: 5 - DNA, 1506 bp, Homo sapiens
atggctgcagctagcgtgaccccccctggctccctggagttgctacagcccggcttctccaagaccctcctggggaccaagctggaagccaagtacctgtgctccgcctgcagaaacgtcctccgcaggcccttccaggcgcagtgtggccaccggtactgctccttctgcctggccagcatcctcagctctgggcctcagaactgtgctgcctgtgttcacgagggcatatatgaagaaggcatttctattttagaaagcagttcggccttcccagataatgctgcccgcagggaggtggagagcctgccggccgtctgtcccagtgatggatgcacctggaaggggaccctgaaagaatacgagagctgccacgaaggccgctgcccgctcatgctgaccgaatgtcccgcgtgcaaaggcctggtccgccttggtgaaaaggagcgccacctggagcacgagtgcccggagagaagcctgagctgccggcattgccgggcaccctgctgcggagcagacgtgaaggcgcaccacgaggtctgccccaagttccccttaacttgtgacggctgcggcaagaagaagatcccccgggagaagtttcaggaccacgtcaagacttgtggcaagtgtcgagtcccttgcagattccacgccatcggctgcctcgagacggtagagggtgagaaacagcaggagcacgaggtgcagtggctgcgggagcacctggccatgctactgagctcggtgctggaggcaaagcccctcttgggagaccagagccacgcggggtcagagctcctgcagaggtgcgagagcctggagaagaagacggccacttttgagaacattgtctgcgtcctgaaccgggaggtggagagggtggccatgactgccgaggcctgcagccggcagcaccggctggaccaagacaagattgaagccctgagtagcaaggtgcagcagctggagaggagcattggcctcaaggacctggcgatggctgacttggagcagaaggtcttggagatggaggcatccacctacgatggggtcttcatctggaagatctcagacttcgccaggaagcgccaggaagctgtggctggccgcatacccgccatcttctccccagccttctacaccagcaggtacggctacaagatgtgtctgcgtatctacctgaacggcgacggcaccgggcgaggaacacacctgtccctcttctttgtggtgatgaagggcccgaatgacgccctgctgcggtggcccttcaaccagaaggtgaccttaatgctgctcgaccagaataaccgggagcacgtgattgacgccttcaggcccgacgtgacttcatcctcttttcagaggccagtcaacgacatgaacatcgcaagcggctgccccctcttctgccccgtctccaagatggaggcaaagaattcctacgtgcgggacgatgccatcttcatcaaggccattgtggacctgacagggctctaa

SEQ ID NO: 6 - DNA, 1044 bp, Homo sapiens
atggaaacacccttctacggcgatgaggcgctgagcggcctgggcggcggcgccagtggcagcggcggcagcttcgcgtccccgggccgcttgttccccggggcgcccccgacggccgcggccggcagcatgatgaagaaggacgcgctgacgctgagcctgagtgagcaggtggcggcagcgctcaagcctgcggccgcgccgcctcctacccccctgcgcgccgacggcgcccccagcgcggcaccccccgacggcctgctcgcctctcccgacctggggctgctgaagctggcctcccccgagctcgagcgcctcatcatccagtccaacgggctggtcaccaccacgccgacgagctcacagttcctctaccccaaggtggcggccagcgaggagcaggagttcgccgagggcttcgtcaaggccctggaggatttacacaagcagaaccagctcggcgcgggcgcggccgctgccgccgccgccgccgccgccggggggccctcgggcacggccacgggctccgcgccccccggcgagctggccccggcggcggccgcgcccgaagcgcctgtctacgcgaacctgagcagctacgcgggcggcgccgggggcgcggggggcgccgcgacggtcgccttcgctgccgaacctgtgcccttcccgccgccgccacccccaggcgcgttggggccgccgcgcctggctgcgctcaaggacgagccacagacggtgcccgacgtgccgagcttcggcgagagcccgccgttgtcgcccatcgacatggacacgcaggagcgcatcaaggcggagcgcaagcggctgcgcaaccgcatcgccgcctccaagtgccgcaagcgcaagctggagcgcatctcgcgcctggaagagaaagtgaagaccctcaagagtcagaacacggagctggcgtccacggcgagcctgctgcgcgagcaggtggcgcagctcaagcagaaagtcctcagccacgtcaacagcggctgccagctgctgccccagcaccaggtgcccgcgtactga

SEQ ID NO: 7 - DNA, 4124 bp, Homo sapiens
atgagcacggaggcggacgagggcatcactttctctgtgccacccttcgccccctcgggcttctgcaccatccccgagggcggcatctgcaggaggggaggagcggcggcggtgggcgagggcgaggagcaccagctgccaccgccgccgccgggcagtttctggaacgtggagagcgccgctgcccctggcatcggttgtccggcggccacctcctcgagcagtgccacccgaggccggggcagctctgttggcgggggcagccgacggaccacggtggcatatgtgatcaacgaagcgagccaagggcaactggtggtggccgagagcgaggccctgcagagcttgcgggaggcgtgcgagacagtgggcgccaccctggaaccctgcattttgggaaactcgactttggagaaaccaccgtgctggaccgcttttacaatgcagatattgcggtggtggagatgagcgatgccttccggcagccgtccttgttttaccaccttggggtgagagaaagtttcagcatggccaacaacatcatcctctactgcgatactaactcggactctctgcagtcactgaaggaaatcatttgccagaagaatactatgtgcactgggaactacacctttgttccttacatgataactccacataacaaagtctactgctgtgacagcagcttcatgaaggggttgacagagctcatgcaaccgaacttcgagctgcttcttggacccatctgcttacctcttgtggatcgttttattcaacttttgaaggtggcacaagcaagttctagccagtacttccgggaatctatactcaatgacatcaggaaagctcgtaatttatacactggtaaagaattggcagctgagttggcaagaattcggcagcgagtagataatatcgaagtcttgacagcagatattgtcataaatctgttactttcctacagagatatccaggactatgattctattgtgaagctggtagagactttagaaaaactgccaacctttgatttggcctcccatcaccatgtgaagtttcattatgcatttgcactgaataggagaaatctccctggtgacagagcaaaagctcttgatattatgattcccatggtgcaaagcgaaggacaagttgcttcagatatgtattgcctagttggtcgaatctacaaagatatgtttttggactctaatttcacggacactgaaagcagagaccatggagcttcttggttcaaaaaggcatttgaatctgagccaacactacagtcaggaattaattatgcggtcctcctcctggcagctggacaccagtttgaatcttcctttgagctccggaaagttggggtgaagctaagtagtcttcttggtaaaaagggaaacttggaaaaactccagagctactgggaagttggattttttctgggggccagcgtcctagccaatgaccacatgagagtcattcaagcatctgaaaagctttttaaactgaagacaccagcatggtacctcaagtctattgtagagacaattttgatatataagcattttgtgaaactgaccacagaacagcctgtggccaagcaagaacttgtggacttttggatggatttcctggtcgaggccacaaagacagatgttactgtggttaggtttccagtattaatattagaaccaaccaaaatctatcaaccttcttatttgtctatcaacaatgaagttgaggaaaagacaatctctatttggcacgtgcttcctgatgacaagaaaggtatacatgagtggaattttagtgcctcttctgtcaggggagtgagtatttctaaatttgaagaaagatgctgctttctttatgtgcttcacaattctgatgatttccaaatctatttctgtacagaacttcattgtaaaaagttttttgagatggtgaacaccattaccgaagagaaggggagaagcacagaggaaggagactgtgaaagtgacttgctggagtatgactatgaatatgatgaaaatggtgacagagtcgttttaggaaaaggcacttatgggatagtctacgcaggtcgggacttgagcaaccaagtcagaattgctattaaggaaatcccagagagagacagcagatactctcagcccctgcatgaagaaatagcattgcataaacacctgaagcacaaaaatattgtccagtatctgggctctttcagtgagaatggtttcattaaaatcttcatggagcaggtccctggaggaagtctttctgctctccttcgttccaaatggggtccattaaaagacaatgagcaaacaattggcttttatacaaagcaaatactggaaggattaaaatatctccatgacaatcagatagttcaccgggacataaagggtgacaatgtgttgattaatacctacagtggtgttctcaagatctctgacttcggaacatcaaagaggcttgctggcataaacccctgtactgaaacttttactggtaccctccagtatatggcaccagaaataatagataaaggaccaagaggctacggaaaagcagcagacatctggtctctgggctgtacaatcattgaaatggccacaggaaaacccccattttatgaactgggagaaccacaagcagctatgttcaaggtgggaatgtttaaagtccaccctgagatcccagagtccatgtctgcagaggccaaggcattcatactgaaatgttttgaaccagatcctgacaagagagcctgtgctaacgacttgcttgttgatgagtttttaaaagtttcaagcaaaaagaaaaagacacaacctaagctttcagctctttcagctggatcaaatgaatatctcaggagtatatccttgccggtacctgtgctggtggaggacaccagcagcagcagtgagtacggctcagtttcacccgacacggagttgaaagtggaccccttctctttcaaaacaagagccaagtcctgcggagaaagagatgtcaagggaattcggacactctttttgggcattccagatgagaattttgaagatcacagtgctcctccttcccctgaagaaaaagattctggattcttcatgctgaggaaggacagtgagaggcgagctacccttcacaggatcctgacggaagaccaagacaaaattgtgagaaacctaatggaatctttagctcagggggctgaagaaccgaaactaaaatgggaacacatcacaaccctcattgcaagcctcagagaatttgtgagatccactgaccgaaaaatcatagccaccacactgtcaaagctgaaactggagctggacttcgacagccatggcattagccaagtccaggtggtactctttggttttcaagatgctgtcaataaagttcttcggaatcataacatcaagccgcactggatgtttgccttagacagtatcattcggaaggcggtacagacagccattaccatcctggttccagaactaaggccacatttcagccttgcatctgagagtgatactgctgatcaagaagacttggatgtagaagatgaccatgaggaacagccttcaaatcaaactgtccgaagacctcaggctgtcattgaagatgctgtggctacctcaggcgtgagcacgctcagttctactgtgtctcatgattcccagagtgctcaccggtcactgaatgtacagcttggaaggatgaaaatagaaaccaatagattactggaagaattggttcggaaagagaaagaattacaagcactccttcatcgagctattgaagaaaaagaccaagaaattaaacacctgaagcttaagtcccaacccatagaaattcctgaattgcctgtatttcatctaaattcttctggcacaaatactgaagattctgaacttaccgactggctgagagtgaatggagctgatgaagacactataagccggtttttggctgaagattatacactattggatgttctctactatgttacacgtgatgacttaaaatgcttgagactaaggggagggatgctgtgcacactgtggaaggctatcattgactttcgaaacaaacagacttga

SEQ ID NO: 8 - DNA, 1128 bp, Homo sapiens
atggagcgcgcgtcctgcttgttgctgctgctgctgccgctggtgcacgtctctgcgaccacgccagaaccttgtgagctggacgatgaagatttccgctgcgtctgcaacttctccgaacctcagcccgactggtccgaagccttccagtgtgtgtctgcagtagaggtggagatccatgccggcggtctcaacctagagccgtttctaaagcgcgtcgatgcggacgccgacccgcggcagtatgctgacacggtcaaggctctccgcgtgcggcggctcacagtgggagccgcacaggttcctgctcagctactggtaggcgccctgcgtgtgctagcgtactcccgcctcaaggaactgacgctcgaggacctaaagataaccggcaccatgcctccgctgcctctggaagccacaggacttgcactttccagcttgcgcctacgcaacgtgtcgtgggcgacagggcgttcttggctcgccgagctgcagcagtggctcaagccaggcctcaaggtactgagcattgcccaagcacactcgcctgccttttcctgcgaacaggttcgcgccttcccggcccttaccagcctagacctgtctgacaatcctggactgggcgaacgcggactgatggcggctctctgtccccacaagttcccggccatccagaatctagcgctgcgcaacacaggaatggagacgcccacaggcgtgtgcgccgcactggcggcggcaggtgtgcagccccacagcctagacctcagccacaactcgctgcgcgccaccgtaaaccctagcgctccgagatgcatgtggtccagcgccctgaactccctcaatctgtcgttcgctgggctggaacaggtgcctaaaggactgccagccaagctcagagtgctcgatctcagctgcaacagactgaacagggcgccgcagcctgacgagctgcccgaggtggataacctgacactggacgggaatcccttcctggtccctggaactgccctcccccacgagggctcaatgaactccggcgtggtcccagcctgtgcacgttcgaccctgtcggtgggggtgtcgggaaccctggtgctgctccaaggggcccggggctttgcctaa

SEQ ID NO: 9 - DNA, 1140 bp, Homo sapiens
atggcggcggcggcggctcaggggggcgggggcggggagccccgtagaaccgagggggtcggcccgggggtcccgggggaggtggagatggtgaaggggcagccgttcgacgtgggcccgcgctacacgcagttgcagtacatcggcgagggcgcgtacggcatggtcagctcggcctatgaccacgtgcgcaagactcgcgtggccatcaagaagatcagccccttcgaacatcagacctactgccagcgcacgctccgggagatccagatcctgctgcgcttccgccatgagaatgtcatcggcatccgagacattctgcgggcgtccaccctggaagccatgagagatgtctacattgtgcaggacctgatggagactgacctgtacaagttgctgaaaagccagcagctgagcaatgaccatatctgctacttcctctaccagatcctgcggggcctcaagtacatccactccgccaacgtgctccaccgagatctaaagccctccaacctgctcatcaacaccacctgcgaccttaagatttgtgatttcggcctggcccggattgccgatcctgagcatgaccacaccggcttcctgacggagtatgtggctacgcgctggtaccgggccccagagatcatgctgaactccaagggctataccaagtccatcgacatctggtctgtgggctgcattctggctgagatgctctctaaccggcccatcttccctggcaagcactacctggatcagctcaaccacattctgggcatcctgggctccccatcccaggaggacctgaattgtatcatcaacatgaaggcccgaaactacctacagtctctgccctccaagaccaaggtggcttgggccaagcttttccccaagtcagactccaaagcccttgacctgctggaccggatgttaacctttaaccccaataaacggatcacagtggaggaagcgctggctcacccctacctggagcagtactatgacccgacggatgagccagtggccgaggagcccttcaccttcgccatggagctggatgacctacctaaggagcggctgaaggagctcatcttccaggagacagcacgcttccagcccggagtgctggaggccccctag

SEQ ID NO: 10 - DNA, 1083 bp, Homo sapiens
atggcggcggcggcggcggcgggcgcgggcccggagatggtccgcgggcaggtgttcgacgtggggccgcgctacaccaacctctcgtacatcggcgagggcgcctacggcatggtgtgctctgcttatgataatgtcaacaaagttcgagtagctatcaagaaaatcagcccctttgagcaccagacctactgccagagaaccctgagggagataaaaatcttactgcgcttcagacatgagaacatcattggaatcaatgacattattcgagcaccaaccatcgagcaaatgaaagatgtatatatagtacaggacctcatggaaacagatctttacaagctcttgaagacacaacacctcagcaatgaccatatctgctattttctctaccagatcctcagagggttaaaatatatccattcagctaacgttctgcaccgtgacctcaagccttccaacctgctgctcaacaccacctgtgatctcaagatctgtgactttggcctggcccgtgttgcagatccagaccatgatcacacagggttcctgacagaatatgtggccacacgttggtacagggctccagaaattatgttgaattccaagggctacaccaagtccattgatatttggtctgtaggctgcattctggcagaaatgctttctaacaggcccatctttccagggaagcattatcttgaccagctgaaccacattttgggtattcttggatccccatcacaagaagacctgaattgtataataaatttaaaagctaggaactatttgctttctcttccacacaaaaataaggtgccatggaacaggctgttcccaaatgctgactccaaagctctggacttattggacaaaatgttgacattcaacccacacaagaggattgaagtagaacaggctctggcccacccatatctggagcagtattacgacccgagtgacgagcccatcgccgaagcaccattcaagttcgacatggaattggatgacttgcctaaggaaaagctcaaagaactaatttttgaagagactgctagattccagccaggatacagatcttaa

SEQ ID NO: 11 - DNA, 2265 bp, Homo sapiens
atgttttcaggggggtgtcatagccccgggtttggccgccccagccccgccttccccgccccggggagcccgccccctgccccgcgtccctgccgacaggaaacaggtgagcagattgccatcaagcagtgccggcaggagctcagcccccggaaccgagagcggtggtgcctggagatccagatcatgagaaggctgacccaccccaatgtggtggctgcccgagatgtccctgaggggatgcagaacttggcgcccaatgacctgcccctgctggccatggagtactgccaaggaggagatctccggaagtacctgaaccagtttgagaactgctgtggtctgcgggaaggtgccatcctcaccttgctgagtgacattgcctctgcgcttagataccttcatgaaaacagaatcatccatcgggatctaaagccagaaaacatcgtcctgcagcaaggagaacagaggttaatacacaaaattattgacctaggatatgccaaggagctggatcagggcagtctttgcacatcattcgtggggaccctgcagtacctggccccagagctactggagcagcagaagtacacagtgaccgtcgactactggagcttcggcaccctggcctttgagtgcatcacgggcttccggcccttcctccccaactggcagcccgtgcagtggcattcaaaagtgcggcagaagagtgaggtggacattgttgttagcgaagacttgaatggaacggtgaagttttcaagctctttaccctaccccaataatcttaacagtgtcctggctgagcgactggagaagtggctgcaactgatgctgatgtggcacccccgacagaggggcacggatcccacgtatgggcccaatggctgcttcaaggccctggatgacatcttaaacttaaagctggttcatatcttgaacatggtcacgggcaccatccacacctaccctgtgacagaggatgagagtctgcagagcttgaaggccagaatccaacaggacacgggcatcccagaggaggaccaggagctgctgcaggaagcgggcctggcgttgatccccgataagcctgccactcagtgtatttcagacggcaagttaaatgagggccacacattggacatggatcttgtttttctctttgacaacagtaaaatcacctatgagactcagatctccccacggccccaacctgaaagtgtcagctgtatccttcaagagcccaagaggaatctcgccttcttccagctgaggaaggtgtggggccaggtctggcacagcatccagaccctgaaggaagattgcaaccggctgcagcagggacagcgagccgccatgatgaatctcctccgaaacaacagctgcctctccaaaatgaagaattccatggcttccatgtctcagcagctcaaggccaagttggatttcttcaaaaccagcatccagattgacctggagaagtacagcgagcaaaccgagtttgggatcacatcagataaactgctgctggcctggagggaaatggagcaggctgtggagctctgtgggcgggagaacgaagtgaaactcctggtagaacggatgatggctctgcagaccgacattgtggacttacagaggagccccatgggccggaagcaggggggaacgctggacgacctagaggagcaagcaagggagctgtacaggagactaagggaaaaacctcgagaccagcgaactgagggtgacagtcaggaaatggtacggctgctgcttcaggcaattcagagcttcgagaagaaagtgcgagtgatctatacgcagctcagtaaaactgtggtttgcaagcagaaggcgctggaactgttgcccaaggtggaagaggtggtgagcttaatgaatgaggatgagaagactgttgtccggctgcaggagaagcggcagaaggagctctggaatctcctgaagattgcttgtagcaaggtccgtggtcctgtcagtggaagcccggatagcatgaatgcctctcgacttagccagcctgggcagctgatgtctcagccctccacggcctccaacagcttacctgagccagccaagaagagtgaagaactggtggctgaagcacataacctctgcaccctgctagaaaatgccatacaggacactgtgagggaacaagaccagagtttcacggccctagactggagctggttacagacggaagaagaagagcacagctgcctggagcaggcctcatga

SEQ ID NO: 12 - DNA, 954 bp, Homo sapiens
atgttccaggcggccgagcgcccccaggagtgggccatggagggcccccgcgacgggctgaagaaggagcggctactggacgaccgccacgacagcggcctggactccatgaaagacgaggagtacgagcagatggtcaaggagctgcaggagatccgcctcgagccgcaggaggtgccgcgcggctcggagccctggaagcagcagctcaccgaggacggggactcgttcctgcacttggccatcatccatgaagaaaaggcactgaccatggaagtgatccgccaggtgaagggagacctggccttcctcaacttccagaacaacctgcagcagactccactccacttggctgtgatcaccaaccagccagaaattgctgaggcacttctgggagctggctgtgatcctgagctccgagactttcgaggaaatacccccctacaccttgcctgtgagcagggctgcctggccagcgtgggagtcctgactcagtcctgcaccaccccgcacctccactccatcctgaaggctaccaactacaatggccacacgtgtctacacttagcctctatccatggctacctgggcatcgtggagcttttggtgtccttgggtgctgatgtcaatgctcaggagccctgtaatggccggactgcccttcacctcgcagtggacctgcaaaatcctgacctggtgtcactcctgttgaagtgtggggctgatgtcaacagagttacctaccagggctattctccctaccagctcacctggggccgcccaagcacccggatacagcagcagctgggccagctgacactagaaaaccttcagatgctgccagagagtgaggatgaggagagctatgacacagagtcagagttcacggagttcacagaggacgagctgccctatgatgactgtgtgtttggaggccagcgtctgacgttatga

SEQ ID NO: 13 - DNA, 2139 bp, Homo sapiens
atggccggggggccgggcccgggggagcccgcagcccccggcgcccagcacttcttgtacgaggtgccgccctgggtcatgtgccgcttctacaaagtgatggacgccctggagcccgccgactggtgccagttcgccgccctgatcgtgcgcgaccagaccgagctgcggctgtgcgagcgctccgggcagcgcacggccagcgtcctgtggccctggatcaaccgcaacgcccgtgtggccgacctcgtgcacatcctcacgcacctgcagctgctccgtgcgcgggacatcatcacagcctggcaccctcccgccccgcttccgtccccaggcaccactgccccgaggcccagcagcatccctgcacccgccgaggccgaggcctggagcccccggaagttgccatcctcagcctccaccttcctctccccagcttttccaggctcccagacccattcagggcctgagctcggcctggtcccaagccctgcttccctgtggcctccaccgccatctccagccccttcttctaccaagccaggcccagagagctcagtgtccctcctgcagggagcccgcccctttccgttttgctggcccctctgtgagatttcccggggcacccacaacttctcggaggagctcaagatcggggagggtggctttgggtgcgtgtaccgggcggtgatgaggaacacggtgtatgctgtgaagaggctgaaggagaacgctgacctggagtggactgcagtgaagcagagcttcctgaccgaggtggagcagctgtccaggtttcgtcacccaaacattgtggactttgctggctactgtgctcagaacggcttctactgcctggtgtacggcttcctgcccaacggctccctggaggaccgtctccactgccagacccaggcctgcccacctctctcctggcctcagcgactggacatccttctgggtacagcccgggcaattcagtttctacatcaggacagccccagcctcatccatggagacatcaagagttccaacgtccttctggatgagaggctgacacccaagctgggagactttggcctggcccggttcagccgctttgccgggtccagccccagccagagcagcatggtggcccggacacagacagtgcggggcaccctggcctacctgcccgaggagtacatcaagacgggaaggctggctgtggacacggacaccttcagctttggggtggtagtgctagagaccttggctggtcagagggctgtgaagacgcacggtgccaggaccaagtatctgaaagacctggtggaagaggaggctgaggaggctggagtggctttgagaagcacccagagcacactgcaagcaggtctggctgcagatgcctgggctgctcccatcgccatgcagatctacaagaagcacctggaccccaggcccgggccctgcccacctgagctgggcctgggcctgggccagctggcctgctgctgcctgcaccgccgggccaaaaggaggcctcctatgacccaggtgtacgagaggctagagaagctgcaggcagtggtggcgggggtgcccgggcattcggaggccgccagctgcatccccccttccccgcaggagaactcctacgtgtccagcactggcagagcccacagtggggctgctccatggcagcccctggcagcgccatcaggagccagtgcccaggcagcagagcagctgcagagaggccccaaccagcccgtggagagtgacgagagcctaggcggcctctctgctgccctgcgctcctggcacttgactccaagctgccctctggacccagcacccctcagggaggccggctgtcctcagggggacacggcaggagaatcgagctgggggagtggcccaggatcccggcccacagccgtggaaggactggcccttggcagctctgcatcatcgtcgtcagagccaccgcagattatcatcaaccctgcccgacagaagatggtccagaagctggccctgtacgaggatggggccctggacagcctgcagctgctgtcgtccagctccctcccaggcttgggcctggaacaggacaggcaggggcccgaagaaagtgatgaatttcagagctga

SEQ ID NO: 14 - DNA, 1284 bp, Homo sapiens
atgagcagaagcaagcgtgacaacaatttttatagtgtagagattggagattctacattcacagtcctgaaacgatatcagaatttaaaacctataggctcaggagctcaaggaatagtatgcgcagcttatgatgccattcttgaaagaaatgttgcaatcaagaagctaagccgaccatttcagaatcagactcatgccaagcgggcctacagagagctagttcttatgaaatgtgttaatcacaaaaatataattggccttttgaatgttttcacaccacagaaatccctagaagaatttcaagatgtttacatagtcatggagctcatggatgcaaatctttgccaagtgattcagatggagctagatcatgaaagaatgtcctaccttctctatcagatgctgtgtggaatcaagcaccttcattctgctggaattattcatcgggacttaaagcccagtaatatagtagtaaaatctgattgcactttgaagattcttgacttcggtctggccaggactgcaggaacgagttttatgatgacgccttatgtagtgactcgctactacagagcacccgaggtcatccttggcatgggctacaaggaaaacgttgacatttggtcagttgggtgcatcatgggagaaatgatcaaaggtggtgttttgttcccaggtacagatcatattgatcagtggaataaagttattgaacagcttggaacaccatgtcctgaattcatgaagaaactgcaaccaacagtaaggacttacgttgaaaacagacctaaatatgctggatatagctttgagaaactcttccctgatgtccttttcccagctgactcagaacacaacaaacttaaagccagtcaggcaagggatttgttatccaaaatgctggtaatagatgcatctaaaaggatctctgtagatgaagctctccaacacccgtacatcaatgtctggtatgatccttctgaagcagaagctccaccaccaaagatccctgacaagcagttagatgaaagggaacacacaatagaagagtggaaagaattgatatataaggaagttatggacttggaggagagaaccaagaatggagttatacgggggcagccctctcctttaggtgcagcagtgatcaatggctctcagcatccatcatcatcgtcgtctgtcaatgatgtgtcttcaatgtcaacagatccgactttggcctctgatacagacagcagtctagaagcagcagctgggcctctgggctgctgtagatga

SEQ ID NO: 15 - DNA, 1446 bp, Homo sapiens
atgggggccttggccagagccctgccgtccatactgctggcattgctgcttacgtccaccccagaggctctgggtgccaaccccggcttggtcgccaggatcaccgacaagggactgcagtatgcggcccaggaggggctattagctctgcagagtgagctgctcaggatcacgctgcctgacttcaccggggacttgaggatcccccacgtcggccgtgggcgctatgagttccacagcctgaacatccacagctgtgagctgcttcactctgcgctgaggcctgtccctggccagggcctgagtctcagcatctccgactcctccatccgggtccagggcaggtggaaggtgcgcaagtcattcttcaaactacagggctcctttgatgtcagtgtcaagggcatcagcatttcggtcaacctcctgttgggcagcgagtcctccgggaggcccacagttactgcctccagctgcagcagtgacatcgctgacgtggaggtggacatgtcgggagacttggggtggctgttgaacctcttccacaaccagattgagtccaagttccagaaagtactggagagcaggatttgcgaaatgatccagaaatcggtgtcctccgatctacagccttatctccaaactctgccagttacaacagagattgacagtttcgccgacattgattatagcttagtggaagcccctcgggcaacagcccagatgctggaggtgatgtttaagggtgaaatctttcatcgtaaccaccgttctccagttaccctccttgctgcagtcatgagccttcctgaggaacacaacaaaatggtctactttgccatctcggattatgtcttcaacacggccagcctggtttatcatgaggaaggatatctgaacttctccatcacagatgacatgataccgcctgactctaatatccgactgaccaccaagtccttccgacccttcgtcccacggttagccaggctctaccccaacatgaacctggaactccagggatcagtgccctctgctccgctcctgaacttcagccctgggaatctgtctgtggacccctatatggagatagatgcctttgtgctcctgcccagctccagcaaggagcctgtcttccggctcagtgtggccactaatgtgtccgccaccttgaccttcaataccagcaagatcactgggttcctgaagccaggaaaggtaaaagtggaactgaaagaatccaaagttggactattcaatgcagagctgttggaagcgctcctcaactattacatccttaacaccctctaccccaagttcaatgataagttggccgaaggcttcccccttcctctgctgaagcgtgttcagctctacgaccttgggctgcagatccataaggacttcctgttcttgggtgccaatgtccaatacatgagagtttga

SEQ ID NO: 16 - DNA, 1182 bp, Homo sapiens
atgcccaagaagaagccgacgcccatccagctgaacccggcccccgacggctctgcagttaacgggaccagctctgcggagaccaacttggaggccttgcagaagaagctggaggagctagagcttgatgagcagcagcgaaagcgccttgaggcctttcttacccagaagcagaaggtgggagaactgaaggatgacgactttgagaagatcagtgagctgggggctggcaatggcggtgtggtgttcaaggtctcccacaagccttctggcctggtcatggccagaaagctaattcatctggagatcaaacccgcaatccggaaccagatcataagggagctgcaggttctgcatgagtgcaactctccgtacatcgtgggcttctatggtgcgttctacagcgatggcgagatcagtatctgcatggagcacatggatggaggttctctggatcaagtcctgaagaaagctggaagaattcctgaacaaattttaggaaaagttagcattgctgtaataaaaggcctgacatatctgagggagaagcacaagatcatgcacagagatgtcaagccctccaacatcctagtcaactcccgtggggagatcaagctctgtgactttggggtcagcgggcagctcatcgactccatggccaactccttcgtgggcacaaggtcctacatgtcgccagaaagactccaggggactcattactctgtgcagtcagacatctggagcatgggactgtctctggtagagatggcggttgggaggtatcccatccctcctccagatgccaaggagctggagctgatgtttgggtgccaggtggaaggagatgcggctgagaccccacccaggccaaggacccccgggaggccccttagctcatacggaatggacagccgacctcccatggcaatttttgagttgttggattacatagtcaacgagcctcctccaaaactgcccagtggagtgttcagtctggaatttcaagattttgtgaataaatgcttaataaaaaaccccgcagagagagcagatttgaagcaactcatggttcatgcttttatcaagagatctgatgctgaggaagtggattttgcaggttggctctgctccaccatcggccttaaccagcccagcacaccaacccatgctgctggcgtctaa

SEQ ID NO: 17 - DNA, 1203 bp, Homo sapiens
atgctggcccggaggaagccggtgctgccggcgctcaccatcaaccctaccatcgccgagggcccatcccctaccagcgagggcgcctccgaggcaaacctggtggacctgcagaagaagctggaggagctggaacttgacgagcagcagaagaagcggctggaagcctttctcacccagaaagccaaggtcggcgaactcaaagacgatgacttcgaaaggatctcagagctgggcgcgggcaacggcggggtggtcaccaaagtccagcacagaccctcgggcctcatcatggccaggaagctgatccaccttgagatcaagccggccatccggaaccagatcatccgcgagctgcaggtcctgcacgaatgcaactcgccgtacatcgtgggcttctacggggccttctacagtgacggggagatcagcatttgcatggaacacatggacggcggctccctggaccaggtgctgaaagaggccaagaggattcccgaggagatcctggggaaagtcagcatcgcggttctccggggcttggcgtacctccgagagaagcaccagatcatgcaccgagatgtgaagccctccaacatcctcgtgaactctagaggggagatcaagctgtgtgacttcggggtgagcggccagctcatcgactccatggccaactccttcgtgggcacgcgctcctacatggctccggagcggttgcagggcacacattactcggtgcagtcggacatctggagcatgggcctgtccctggtggagctggccgtcggaaggtaccccatccccccgcccgacgccaaagagctggaggccatctttggccggcccgtggtcgacggggaagaaggagagcctcacagcatctcgcctcggccgaggccccccgggcgccccgtcagcggtcacgggatggatagccggcctgccatggccatctttgaactcctggactatattgtgaacgagccacctcctaagctgcccaacggtgtgttcacccccgacttccaggagtttgtcaataaatgcctcatcaagaacccagcggagcgggcggacctgaagatgctcacaaaccacaccttcatcaagcggtccgaggtggaagaagtggattttgccggctggttgtgtaaaaccctgcggctgaaccagcccggcacacccacgcgcaccgccgtgtga

SEQ ID NO: 18 - DNA, 957 bp, Homo sapiens
atgtccaagccacccgcacccaaccccacacccccccggaacctggactcccggaccttcatcaccattggagacagaaactttgaggtggaggctgatgacttggtgaccatctcagaactgggccgtggagcctatggggtggtagagaaggtgcggcacgcccagagcggcaccatcatggccgtgaagcggatccgggccaccgtgaactcacaggagcagaagcggctgctcatggacctggacatcaacatgcgcacggtcgactgtttctacactgtcaccttctacggggcactattcagagagggagacgtgtggatctgcatggagctcatggacacatccttggacaagttctaccggaaggtgctggataaaaacatgacaattccagaggacatccttggggagattgctgtgtctatcgtgcgggccctggagcatctgcacagcaagctgtcggtgatccacagagatgtgaagccctccaatgtccttatcaacaaggagggccatgtgaagatgtgtgactttggcatcagtggctacttggtggactctgtggccaagacgatggatgccggctgcaagccctacatggcccctgagaggatcaacccagagctgaaccagaagggctacaatgtcaagtccgacgtctggagcctgggcatcaccatgattgagatggccatcctgcggttcccttacgagtcctgggggaccccgttccagcagctgaagcaggtggtggaggagccgtccccccagctcccagccgaccgtttctcccccgagtttgtggacttcactgctcagtgcctgaggaagaaccccgcagagcgtatgagctacctggagctgatggagcaccccttcttcaccttgcacaaaaccaagaagacggacattgctgccttcgtgaaggagatcctgggagaagactcatag

SEQ ID NO: 19 - DNA, 1005 bp, Homo sapiens
atgtctcagtcgaaaggcaagaagcgaaaccctggccttaaaattccaaaagaagcatttgaacaacctcagaccagttccacaccacctcgagatttagactccaaggcttgcatttctattggaaatcagaactttgaggtgaaggcagatgacctggagcctataatggaactgggacgaggtgcgtacggggtggtggagaagatgcggcacgtgcccagcgggcagatcatggcagtgaagcggatccgagccacagtaaatagccaggaacagaaacggctactgatggatttggatatttccatgaggacggtggactgtccattcactgtcaccttttatggcgcactgtttcgggagggtgatgtgtggatctgcatggagctcatggatacatcactagataaattctacaaacaagttattgataaaggccagacaattccagaggacatcttagggaaaatagcagtttctattgtaaaagcattagaacatttacatagtaagctgtctgtcattcacagagacgtcaagccttctaatgtactcatcaatgctctcggtcaagtgaagatgtgcgattttggaatcagtggctacttggtggactctgttgctaaaacaattgatgcaggttgcaaaccatacatggcccctgaaagaataaacccagagctcaaccagaagggatacagtgtgaagtctgacatttggagtctgggcatcacgatgattgagttggccatccttcgatttccctatgattcatggggaactccatttcagcagctcaaacaggtggtagaggagccatcgccacaactcccagcagacaagttctctgcagagtttgttgactttacctcacagtgcttaaagaagaattccaaagaacggcctacatacccagagctaatgcaacatccatttttcaccctacatgaatccaaaggaacagatgtggcatcttttgtaaaactgattcttggagactaa

SEQ ID NO: 20 - DNA, 4539 bp, Homo sapiens
atggcggcggcggcggggaatcgcgcctcgtcgtcgggattcccgggcgccagggctacgagccctgaggcaggcggcggcggaggagccctcaaggcgagcagcgcgcccgcggctgccgcgggactgctgcgggaggcgggcagcgggggccgcgagcgggcggactggcggcggcggcagctgcgcaaagtgcggagtgtggagctggaccagctgcctgagcagccgctcttccttgccgcctcaccgccggcctcctcgacttccccgtcgccggagcccgcggacgcagcggggagtgggaccggcttccagcctgtggcggtgccgccgccccacggagccgcgagccgcggcggcgcccaccttaccgagtcggtggcggcgccggacagcggcgcctcgagtcccgcagcggccgagcccggggagaagcgggcgcccgccgccgagccgtctcctgcagcggcccccgccggtcgtgagatggagaataaagaaactctcaaagggttgcacaagatggatgatcgtccagaggaacgaatgatcagggagaaactgaaggcaacctgtatgccagcctggaagcacgaatggttggaaaggagaaataggcgagggcctgtggtggtaaaaccaatcccagttaaaggagatggatctgaaatgaatcacttagcagctgagtctccaggagaggtccaggcaagtgcggcttcaccagcttccaaaggccgacgcagtccttctcctggcaactccccatcaggtcgcacagtgaaatcagaatctccaggagtaaggagaaaaagagtttccccagtgccttttcagagtggcagaatcacaccaccccgaagagccccttcaccagatggcttctcaccatatagccctgaggaaacaaaccgccgtgttaacaaagtgatgcgggccagactgtacttactgcagcagatagggcctaactctttcctgattggaggagacagcccagacaataaataccgggtgtttattgggcctcagaactgcagctgtgcacgtggaacattctgtattcatctgctatttgtgatgctccgggtgtttcaactagaaccttcagacccaatgttatggagaaaaactttaaagaattttgaggttgagagtttgttccagaaatatcacagtaggcgtagctcaaggatcaaagctccatctcgtaacaccatccagaagtttgtttcacgcatgtcaaattctcatacattgtcatcatctagtacttctacgtctagttcagaaaacagcataaaggatgaagaggaacagatgtgtcctatttgcttgttgggcatgcttgatgaagaaagtcttacagtgtgtgaagacggctgcaggaacaagctgcaccaccactgcatgtcaatttgggcagaagagtgtagaagaaatagagaacctttaatatgtcccctttgtagatctaagtggagatctcatgatttctacagccacgagttgtcaagtcctgtggattccccttcttccctcagagctgcacagcagcaaaccgtacagcagcagcctttggctggatcacgaaggaatcaagagagcaattttaaccttactcattatggaactcagcaaatccctcctgcttacaaagatttagctgagccatggattcaggtgtttggaatggaactcgttggctgcttattttctagaaactggaatgtgagagagatggccctcaggcgtctttcccatgatgtcagtggggccctgctgttggcaaatggggagagcactggaaattctgggggcagcagtggaagcagcccgagtgggggagccaccagtgggtcttcccagaccagtatctcaggagatgtggtggaggcatgctgcagcgttctgtcaatggtctgtgctgaccctgtctacaaagtgtacgttgctgctttaaaaacattgagagccatgctggtatatactccttgccacagtttagcggaaagaatcaaacttcagagacttctccagccagttgtagacaccatcctagtcaaatgtgcagatgccaatagccgcacaagtcagctgtccatatcaacactgttggaactgtgcaaaggccaagcaggagagttggcagttggcagagaaatactaaaagctggatccattggtattggtggtgttgattatgtcttaaattgtattcttggaaaccaaactgaatcaaacaattggcaagaacttcttggccgcctttgtcttatagatagactgttgttggaatttcctgctgaattttatcctcatattgtcagtactgatgtttcacaagctgagcctgttgaaatcaggtataagaagctgctgtccctcttaacctttgctttgcagtccattgataattcccactcaatggttggcaaactttccagaaggatctacttgagttctgcaagaatggttactacagtaccccatgtgttttcaaaactgttagaaatgctgagtgtttccagttccactcacttcaccaggatgcgtcgccgtttgatggctattgcagatgaggtggaaattgccgaagccatccagttgggcgtagaagacactttggatggtcaacaggacagcttcttgcaggcatctgttcccaacaactatctggaaaccacagagaacagttcccctgagtgcacagtccatttagagaaaactggaaaaggattatgtgctacaaaattgagtgccagttcagaggacatttctgagagactggccagcatttcagtaggaccttctagttcaacaacaacaacaacaacaacaacagagcaaccaaagccaatggttcaaacaaaaggcagaccccacagtcagtgtttgaactcctctcctttatctcatcattcccaattaatgtttccagccttgtcaaccccttcttcttctaccccatctgtaccagctggcactgcaacagatgtctctaagcatagacttcagggattcattccctgcagaataccttctgcatctcctcaaacacagcgcaagttttctctacaattccacagaaactgtcctgaaaacaaagactcagataaactttccccagtctttactcagtcaagacccttgccctccagtaacatacacaggccaaagccatctagacctaccccaggtaatacaagtaaacagggagatccctcaaaaaatagcatgacacttgatctgaacagtagttccaaatgtgatgacagctttggctgtagcagcaatagtagtaatgctgttatacccagtgacgagacagtgttcaccccagtagaggagaaatgcagattagatgtcaatacagagctcaactccagtattgaggaccttcttgaagcatctatgccttcaagtgatacaacagtaacttttaagtcagaagttgctgtcctgtctcctgaaaaggctgaaaatgatgatacctacaaagatgatgtgaatcataatcaaaagtgcaaagagaagatggaagctgaagaagaagaagctttagcaattgccatggcaatgtcagcgtctcaggatgccctccccatagttcctcagctgcaggttgaaaatggagaagatatcatcattattcaacaggatacaccagagactctaccaggacataccaaagcaaaacaaccgtatagagaagacactgaatggctgaaaggtcaacagataggccttggagcattttcttcttgttatcaggctcaagatgtgggaactggaactttaatggctgttaaacaggtgacttatgtcagaaacacatcttctgagcaagaagaagtagtagaagcactaagagaagagataagaatgatgagccatctgaatcatccaaacatcattaggatgttgggagccacgtgtgagaagagcaattacaatctcttcattgaatggatggcagggggatcggtggctcatttgctgagtaaatatggagccttcaaagaatcagtagttattaactacactgaacagttactccgtggcctttcgtatctccatgaaaaccaaatcattcacagagatgtcaaaggtgccaatttgctaattgacagcactggtcagagactaagaattgcagattttggagctgcagccaggttggcatcaaaaggaactggtgcaggagagtttcagggacaattactggggacaattgcatttatggcacctgaggtactaagaggtcaacagtatggaaggagctgtgatgtatggagtgttggctgtgctattatagaaatggcttgtgcaaaaccaccatggaatgcagaaaaacactccaatcatcttgctttgatatttaagattgctagtgcaactactgctccatcgatcccttcacatttgtctcctggtttacgagatgtggctcttcgttgtttagaacttcaacctcaggacagacctccatcaagagagctactgaagcatccagtctttcgtactacatggtag

SEQ ID NO: 21 - DNA, 1974 bp, Homo sapiens
atggacgaacaggaggcattgaactcaatcatgaacgatctggtggccctccagatgaaccgacgtcaccggatgcctggatatgagaccatgaagaacaaagacacaggtcactcaaataggcagaaaaaacacaacagcagcagctcagcccttctgaacagccccacagtaacaacaagctcatgtgcaggggccagtgagaaaaagaaatttttgagtgacgtcagaatcaagttcgagcacaacggggagaggcgaattatagcgttcagccggcctgtgaaatatgaagatgtggagcacaaggtgacaacagtatttggacaacctcttgatctacattacatgaacaatgagctctccatcctgctgaaaaaccaagatgatcttgataaagcaattgacattttagatagaagctcaagcatgaaaagccttaggatattgctgttgtcccaggacagaaaccataacagttcctctccccactctggggtgtccagacaggtgcggatcaaggcttcccagtccgcaggggatataaatactatctaccagccccccgagcccagaagcaggcacctctctgtcagctcccagaaccctggccgaagctcacctccccctggctatgttcctgagcggcagcagcacattgcccggcaggggtcctacaccagcatcaacagtgagggggagttcatcccagagaccagcgagcagtgcatgctggatcccctgagcagtgcagaaaattccttgtctggaagctgccaatccttggacaggtcagcagacagcccatccttccggaaatcacgaatgtcccgtgcccagagcttccctgacaacagacaggaatactcagatcgggaaactcagctttatgacaaaggggtcaaaggtggaacctacccccggcgctaccacgtgtctgtgcaccacaaggactacagtgatggcagaagaacatttccccgaatacggcgtcatcaaggcaacttgttcaccctggtgccctccagccgctccctgagcacaaatggcgagaacatgggtctggctgtgcaatacctggacccccgtgggcgcctgcggagtgcggacagcgagaatgccctctctgtgcaggagaggaatgtgccaaccaagtctcccagtgcccccatcaactggcgccggggaaagctcctgggccagggtgccttcggcagggtctatttgtgctatgacgtggacacgggacgtgaacttgcttccaagcaggtccaatttgatccagacagtcctgagacaagcaaggaggtgagtgctctggagtgcgagatccagttgctaaagaacttgcagcatgagcgcatcgtgcagtactatggctgtctgcgggaccgcgctgagaagaccctgaccatcttcatggagtacatgccagggggctcggtgaaagaccagttgaaggcttacggtgctctgacagagagcgtgacccgaaagtacacgcggcagatcctggagggcatgtcctacctgcacagcaacatgattgttcaccgggacattaagggagccaacatcctccgagactctgctgggaatgtaaagctgggggactttggggccagcaaacgcctgcagacgatctgtatgtcggggacgggcatgcgctccgtcactggcacaccctactggatgagccctgaggtgatcagcggcgagggctatggaaggaaagcagacgtgtggagcctgggctgcactgtggtggagatgctgacagagaaaccaccgtgggcagagtatgaagctatggccgccatcttcaagattgccacccagcccaccaatcctcagctgccctcccacatctctgaacatggccgggacttcctgaggcgcatttttgtggaggctcgccagagaccttcagctgaggagctgctcacacaccactttgcacagctcatgtactga

SEQ ID NO: 22 - DNA, 4826 bp, Homo sapiens
atgagagaagccgctgccgcgctggtccctcctcccgcctttgccgtcacgcctgccgccgccatggaggagccgccgccaccgccgccgccgccaccaccgccaccggaacccgagaccgagtcagaacccgagtgctgcttggcggcgaggcaagagggcacattgggagattcagcttgcaagagtcctgaatctgatctagaagacttctccgatgaaacaaatacagagaatctttatggtacctctccccccagcacacctcgacagatgaaacgcatgtcaaccaaacatcagaggaataatgtggggaggccagccagtcggtctaatttgaaagaaaaaatgaatgcaccaaatcagcctccacataaagacactggaaaaacagtggagaatgtggaagaatacagctataagcaggagaaaaagatccgagcagctcttagaacaacagagcgtgatcataaaaaaaatgtacagtgctcattcatgttagactcagtgggtggatctttgccaaaaaaatcaattccagatgtggatctcaataagccttacctcagccttggctgtagcaatgctaagcttccagtatctgtgcccatgcctatagccagacctgcacgccagacttctaggactgactgtccagcagatcgtttaaagttttttgaaactttacgacttttgctaaagcttacctcagtctcaaagaaaaaagacagggagcaaagaggacaagaaaatacgtctggtttctggcttaaccgatctaacgaactgatctggttagagctacaagcctggcatgcaggacggacaattaacgaccaggacttctttttatatacagcccgtcaagccatcccagatattattaatgaaatccttactttcaaagtcgactatgggagcttcgcctttgttagagatagagctggttttaatggtacttcagtagaagggcagtgcaaagccactcctggaacaaagattgtaggttactcaacacatcatgagcatctccaacgccagagggtctcatttgagcaggtaaaacggataatggagctgctagagtacatagaagcactttatccatcattgcaggctcttcagaaggactatgaaaaatatgctgcaaaagacttccaggacagggtgcaggcactctgtttgtggttaaacatcacaaaagacttaaatcagaaattaaggattatgggcactgttttgggcatcaagaatttatcagacattggctggccagtgtttgaaatcccttcccctcgaccatccaaaggtaatgagccggagtatgagggtgatgacacagaaggagaattaaaggagttggaaagtagtacggatgagagtgaagaagaacaaatctctgatcctagggtaccggaaatcagacagcccatagataacagcttcgacatccagtcgcgggactgcatatccaagaagcttgagaggctcgaatctgaggatgattctcttggctggggagcaccagactggagcacagaagcaggctttagtagacattgtctgacttctatttatagaccatttgtagacaaagcactgaagcagatggggttaagaaagttaattttaagacttcacaagctaatggatggttccttgcaaagggcacgtatagcattggtaaagaacgatcgtccagtggagttttctgaatttccagatcccatgtggggttcagattatgtgcagttgtcaaggacaccaccttcatctgaggagaaatgcagtgctgtgtcgtgggaggagctgaaggccatggatttaccttcattcgaacctgccttcctagttctctgccgagtccttctgaatgtcatacatgagtgtctgaagttaagattggagcagagacctgctggagaaccatctctcttgagtattaagcagctggtgagagagtgtaaggaggtcctgaagggcggcctgctgatgaagcagtactaccagttcatgctgcaggaggttctggaggacttggagaagcccgactgcaacattgacgcttttgaagaggatctacataaaatgcttatggtgtattttgattacatgagaagctggatccaaatgctacagcaattacctcaagcatcgcatagtttaaaaaatctgttagaagaagaatggaatttcaccaaagaaataactcattacatacggggaggagaagcacaggccgggaagcttttctgtgacattgcaggaatgctgctgaaatctacaggaagttttttagaatttggcttacaggagagctgtgctgaattttggactagtgcggatgacagcagtgcttccgacgaaatcaggaggtctgttatagagatcagtcgagccctgaaggagctcttccatgaagccagagaaagggcttccaaagcacttggatttgctaaaatgttgagaaaggacctggaaatagcagcagaattcaggctttcagccccagttagagacctcctggatgttctgaaatcaaaacagtatgtcaaggtgcaaattcctgggttagaaaacttgcaaatgtttgttccagacactcttgctgaggagaagagtattattttgcagttactcaatgcagctgcaggaaaggactgttcaaaagattcagatgacgtactcatcgatgcctatctgcttctgaccaagcacggtgatcgagcccgtgattcagaggacagctggggcacctgggaggcacagcctgtcaaagtcgtgcctcaggtggagactgttgacaccctgagaagcatgcaggtggataatcttttactagttgtcatgcagtctgcgcatctcacaattcagagaaaagctttccagcagtccattgagggacttatgactctgtgccaggagcagacatccagtcagccggtcatcgccaaagctttgcagcagctgaagaatgatgcattggagctatgcaacaggataagcaatgccattgaccgcgtggaccacatgttcacatcagaatttgatgctgaggttgatgaatctgaatctgtcaccttgcaacagtactaccgagaagcaatgattcaggggtacaattttggatttgagtatcataaagaagttgttcgtttgatgtctggggagtttagacagaagataggagacaaatatataagctttgcccggaagtggatgaattatgtcctgactaaatgtgagagtggtagaggtacaagacccaggtgggcgactcaaggatttgattttctacaagcaattgaacctgcctttatttcagctttaccagaagatgacttcttgagtttacaagccttgatgaatgaatgcattggccatgtcataggaaaaccacacagtcctgttacaggtttgtaccttgccattcatcggaacagcccccgtcctatgaaggtacctcgatgccatagtgaccctcctaacccacacctcattatccccactccagagggattcagcactcggagcatgccttccgacgcgcggagccatggcagccctgctgctgctgctgctgctgctgctgctgctgttgctgccagtcggcccagcccctctggtggtgactctgtgctgcccaaatccatcagcagtgcccatgataccaggggttccagcgttcctgaaaatgatcgattggcttccatagctgctgaattgcagtttaggtccctgagtcgtcactcaagccccacggaggagcgagatgaaccagcatatccaagaggagattcaagtgggtccacaagaagaagttgggaacttcggacactaatcagccagagtaaagatactgcttctaaactaggacccatagaagctatccagaagtcagtccgattgtttgaagaaaagaggtaccgagaaatgaggagaaagaatatcattggtcaagtttgtgatacgcctaagtcctatgataatgttatgcacgttggcttgaggaaggtgaccttcaaatggcaaagaggaaacaaaattggagaaggccagtatgggaaggtgtacacctgcatcagcgtcgacaccggggagctgatggccatgaaagagattcgatttcaacctaatgaccataagactatcaaggaaactgcagacgaattgaaaatattcgaaggcatcaaacaccccaatctggttcggtattttggtgtggagctccatagagaagaaatgtacatcttcatggagtactgcgatgaggggactttagaagaggtgtcaaggctgggacttcaggaacatgtgattaggctgtattcaaagcagatcaccattgcgatcaacgtcctccatgagcatggcatagtccaccgtgacattaaaggtgccaatatcttccttacctcatctggattaatcaaactgggagattttggatgttcagtaaagctcaaaaacaatgcccagaccatgcctggtgaagtgaacagcaccctggggacagcagcatacatggcacctgaagtcatcactcgtgccaaaggagagggccatgggcgtgcggccgacatctggagtctggggtgtgttgtcatagagatggtgactggcaagaggccttggcatgagtatgagcacaactttcaaattatgtataaagtggggatgggacataagccaccaatccctgaaagattaagccctgaaggaaaggacttcctttctcactgccttgagagtgacccaaagatgagatggaccgccagccagctcctcgaccattcgtttgtcaaggtttgcacagatgaagaatg

SEQ ID NO: 23 - DNA, 3867 bp, Homo sapiens
atggcggggccgtgtccccggtccggggcggagcgcgccggcagctgctggcaggacccgctggccgtggcgctgagccggggccggcagctcgcggcgcccccgggccggggctgcgcgcggagccggccgctcagcgtggtctacgtgctgacccgggagccgcagcccgggctcgagcctcgggagggaaccgaggcggagccgctgcccctgcgctgcctgcgcgaggcttgcgcgcaggtcccccggccgcggccgcccccgcagctgcgcagcctgcccttcgggacgctggagctaggcgacaccgcggctctggatgccttctacaacgcggatgtggtggtgctggaggtgagcagctcgctggtacagccctccctgttctaccaccttggtgtgcgtgagagcttcagcatgaccaacaatgtgctcctctgctcccaggccgacctccctgacctgcaggccctgcgggaggatgttttccagaagaactcggattgcgttggcagctacacactgatcccctatgtggtgacggccactggtcgggtgctgtgtggtgatgcaggccttctgcggggcctggctgatgggctggtacaggctggagtggggaccgaggccctgctcactcccctggtgggccggcttgcccgcctgctggaggccacacccacagactcttgtggctatttccgggagaccattcggcgggacatccggcaggcgcgggagcggttcagtgggccacagctgcggcaggagctggctcgcctgcagcggagactggacagcgtggagctgctgagccccgacatcatcatgaacttgctgctctcctaccgcgatgtgcaggactactcggccatcattgagctggtggagacgctgcaggccttgcccacctgtgatgtggccgagcagcataatgtctgcttccactacacttttgccctcaaccggaggaacaggcctggggaccgggcgaaggccctgtctgtgctgctgccgctggtacagcttgagggctctgtggcgcccgatctgtactgcatgtgtggccgtatctacaaggacatgttcttcagctcgggtttccaggatgctgggcaccgggagcaggcctatcactggtatcgcaaggcttttgacgtagagcccagccttcactcaggcatcaatgcagctgtgctcctcattgctgccgggcagcactttgaggattccaaagagctccggctaataggcatgaagctgggctgcctgctggcccgcaaaggctgcgtggagaagatgcagtattactgggatgtgggtttctacctgggagcccagatcctcgccaatgaccccacccaggtggtgctggctgcagagcagctgtataagctcaatgcccccatatggtacctggtgtccgtgatggagaccttcctgctctaccagcacttcaggcccacgccagagccccctggagggccaccacgccgtgcccacttctggctccacttcttgctacagtcctgccaaccattcaagacagcctgtgcccagggcgaccagtgcttggtgctggtcctggagatgaacaaggtgctgctgcctgcaaagctcgaggttcggggtactgacccagtaagcacagtgaccctgagcctgctggagcctgagacccaggacattccctccagctggaccttcccagtcgcctccatatgcggagtcagcgcctcaaagcgcgacgagcgctgctgcttcctctatgcactccccccggctcaggacgtccagctgtgcttccccagcgtagggcactgccagtggttctgcggcctgatccaggcctgggtgacgaacccggattccacggcgcccgcggaggaggcggagggcgcgggggagatgttggagtttgattatgagtacacggagacgggcgagcggctggtgctgggcaagggcacgtatggggtggtgtacgcgggccgcgatcgccacacgagggtgcgcatcgccatcaaggagatcccggagcgggacagcaggttctctcagcccctgcatgaagagatcgctcttcacagacgcctgcgccacaagaacatagtgcgctatctgggctcagctagccagggcggctaccttaagatcttcatggaggaagtgcctggaggcagcctgtcctccttgctgcggtcggtgtggggacccctgaaggacaacgagagcaccatcagtttctacacccgccagatcctgcagggacttggctacttgcacgacaaccacatcgtgcacagggacataaaaggggacaatgtgctgatcaacaccttcagtgggctgctcaagatttctgacttcggcacctccaagcggctggcaggcatcacaccttgcactgagaccttcacaggaactctgcagtatatggccccagaaatcattgaccagggcccacgcgggtatgggaaagcagctgacatctggtcactgggctgcactgtcattgagatggccacaggtcgcccccccttccacgagctcgggagcccacaggctgccatgtttcaggtgggtatgtacaaggtccatccgccaatgcccagctctctgtcggccgaggcccaagcctttctcctccgaacttttgagccagacccccgcctccgagccagcgcccagacactgctgggggaccccttcctgcagcctgggaaaaggagccgcagccccagctccccacgacatgctccacggccctcagatgccccttctgccagtcccactccttcagccaactcaaccacccagtctcagacattcccgtgccctcaggcaccctctcagcacccacccagccccccgaagcgctgcctcagttatgggggcaccagccagctccgggtgcccgaggagcctgcggccgaggagcctgcgtctccggaggagagttcggggctgagcctgctgcaccaggagagcaagcgtcgggccatgctggccgcagtattggagcaggagctgccagcgctggcggagaatctgcaccaggagcagaagcaagagcagggggcccgtctgggcagaaaccatgtggaagagctgctgcgctgcctcggggcacacatccacactcccaaccgccggcagctcgcccaggagctgcgggcgctgcaaggacggctgagggcccagggccttgggcctgcgcttctgcacagaccgctgtttgccttcccggatgcggtgaagcagatcctccgcaagcgccagatccgtccacactggatgttcgttctggactcactgctcagccgtgctgtgcgggcagccctgggtgtgctaggaccggaggtggagaaggaggcggtctcaccgaggtcagaggagctgagtaatgaaggggactcccagcagagcccaggccagcagagcccgcttccggtggagcccgagcagggccccgctcctctgatggtgcagctgagcctcttgagggcagagactgatcggctgcgcgaaatcctggcggggaaggaacgggagtaccaggccctggtgcagcgggctctacagcggctgaatgaggaagcccggacctatgtcctggccccagagcctccaactgctctttcaacggaccagggcctggtgcagtggctacaggaactgaatgtggattcaggcaccatccaaatgctgttgaaccatagcttcaccctccacactctgctcacctatgccactcgagatgacctcatctacacccgcatcaggggagggatggtatgccgcatctggagggccatcttggcacagcgagcaggatccacaccagtcacctctggaccctga

SEQ ID NO: 24 - DNA, 1740 bp, Homo sapiens
atgtctacagcctctgccgcctcctcctcctcctcgtcttcggccggtgagatgatcgaagccccttcccaggtcctcaactttgaagagatcgactacaaggagatcgaggtggaagaggttgttggaagaggagcctttggagttgtttgcaaagctaagtggagagcaaaagatgttgctattaaacaaatagaaagtgaatctgagaggaaagcgtttattgtagagcttcggcagttatcccgtgtgaaccatcctaatattgtaaagctttatggagcctgcttgaatccagtgtgtcttgtgatggaatatgctgaagggggctctttatataatgtgctgcatggtgctgaaccattgccatattatactgctgcccacgcaatgagttggtgtttacagtgttcccaaggagtggcttatcttcacagcatgcaacccaaagcgctaattcacagggacctgaaaccaccaaacttactgctggttgcaggggggacagttctaaaaatttgtgattttggtacagcctgtgacattcagacacacatgaccaataacaaggggagtgctgcttggatggcacctgaagtttttgaaggtagtaattacagtgaaaaatgtgacgtcttcagctggggtattattctttgggaagtgataacgcgtcggaaaccctttgatgagattggtggcccagctttccgaatcatgtgggctgttcataatggtactcgaccaccactgataaaaaatttacctaagcccattgagagcctgatgactcgttgttggtctaaagatccttcccagcgcccttcaatggaggaaattgtgaaaataatgactcacttgatgcggtactttccaggagcagatgagccattacagtatccttgtcagtattcagatgaaggacagagcaactctgccaccagtacaggctcattcatggacattgcttctacaaatacgagtaacaaaagtgacactaatatggagcaagttcctgccacaaatgatactattaagcgcttagaatcaaaattgttgaaaaatcaggcaaagcaacagagtgaatctggacgtttaagcttgggagcctcccgtgggagcagtgtggagagcttgcccccaacctctgagggcaagaggatgagtgctgacatgtctgaaatagaagctaggatcgccgcaaccacaggcaacggacagccaagacgtagatccatccaagacttgactgtaactggaacagaacctggtcaggtgagcagtaggtcatccagtcccagtgtcagaatgattactacctcaggaccaacctcagaaaagccaactcgaagtcatccatggacccctgatgattccacagataccaatggatcagataactccatcccaatggcttatcttacactggatcaccaactacagcctctagcaccgtgcccaaactccaaagaatctatggcagtgtttgaacagcattgtaaaatggcacaagaatatatgaaagttcaaacagaaattgcattgttattacagagaaagcaagaactagttgcagaactggaccaggatgaaaaggaccagcaaaatacatctcgcctggtacaggaacataaaaagcttttagatgaaaacaaaagcctttctacttactaccagcaatgcaaaaaacaactagaggtcatcagaagtcagcagcagaaacgacaaggcacttcatga

SEQ ID NO: 25 - DNA, 1113 bp, Homo sapiens
atgctgtccaactcccagggccagagcccgccggtgccgttccccgccccggccccgccgccgcagccccccacccctgccctgccgcaccccccggcgcagccgccgccgccgcccccgcagcagttcccgcagttccacgtcaagtccggcctgcagatcaagaagaacgccatcatcgatgactacaaggtcaccagccaggtcctggggctgggcatcaacggcaaagttttgcagatcttcaacaagaggacccaggagaaattcgccctcaaaatgcttcaggactgccccaaggcccgcagggaggtggagctgcactggcgggcctcccagtgcccgcacatcgtacggatcgtggatgtgtacgagaatctgtacgcagggaggaagtgcctgctgattgtcatggaatgtttggacggtggagaactctttagccgaatccaggatcgaggagaccaggcattcacagaaagagaagcatccgaaatcatgaagagcatcggtgaggccatccagtatctgcattcaatcaacattgcccatcgggatgtcaagcctgagaatctcttatacacctccaaaaggcccaacgccatcctgaaactcactgactttggctttgccaaggaaaccaccagccacaactctttgaccactccttgttatacaccgtactatgtggctccagaagtgctgggtccagagaagtatgacaagtcctgtgacatgtggtccctgggtgtcatcatgtacatcctgctgtgtgggtatccccccttctactccaaccacggccttgccatctctccgggcatgaagactcgcatccgaatgggccagtatgaatttcccaacccagaatggtcagaagtatcagaggaagtgaagatgctcattcggaatctgctgaaaacagagcccacccagagaatgaccatcaccgagtttatgaaccacccttggatcatgcaatcaacaaaggtccctcaaaccccactgcacaccagccgggtcctgaaggaggacaaggagcggtgggaggatgtcaaggggtgtcttcatgacaagaacagcgaccaggccacttggctgaccaggttgtga

SEQ ID NO: 26 - DNA, 954 bp, Homo sapiens
atgcgacccgaccgcgctgaggctccaggaccgcccgccatggctgcaggaggtcccggcgcggggtctgcggccccggtctcctccacatcctcccttcccctggctgctctcaacatgcgagtgcggcgccgcctgtctctgttcttgaacgtgcggacacaggtggcggccgactggaccgcgctggcggaggagatggactttgagtacttggagatccggcaactggagacacaagcggaccccactggcaggctgctggacgcctggcagggacgccctggcgcctctgtaggccgactgctcgagctgcttaccaagctgggccgcgacgacgtgctgctggagctgggacccagcattgaggaggattgccaaaagtatatcttgaagcagcagcaggaggaggctgagaagcctttacaggtggccgctgtagacagcagtgtcccacggacagcagagctggcgggcatcaccacacttgatgaccccctggggcatatgcctgagcgtttcgatgccttcatctgctattgccccagcgacatccagtttgtgcaggagatgatccggcaactggaacagacaaactatcgactgaagttgtgtgtgtctgaccgcgatgtcctgcctggcacctgtgtctggtctattgctagtgagctcatcgaaaagaggttggctagaaggccacggggtgggtgccgccggatggtggtggttgtctctgatgattacctgcagagcaaggaatgtgacttccagaccaaatttgcactcagcctctctccaggtgcccatcagaagcgactgatccccatcaagtacaaggcaatgaagaaagagttccccagcatcctgaggttcatcactgtctgcgactacaccaacccctgcaccaaatcttggttctggactcgccttgccaaggccttgtccctgccctga

SEQ ID NO: 27 - DNA, 2907 bp, Homo sapiens
atggcagaagatgatccatatttgggaaggcctgaacaaatgtttcatttggatccttctttgactcatacaatatttaatccagaagtatttcaaccacagatggcactgccaacagatggcccataccttcaaatattagagcaacctaaacagagaggatttcgtttccgttatgtatgtgaaggcccatcccatggtggactacctggtgcctctagtgaaaagaacaagaagtcttaccctcaggtcaaaatctgcaactatgtgggaccagcaaaggttattgttcagttggtcacaaatggaaaaaatatccacctgcatgcccacagcctggtgggaaaacactgtgaggatgggatctgcactgtaactgctggacccaaggacatggtggtcggcttcgcaaacctgggtatacttcatgtgacaaagaaaaaagtatttgaaacactggaagcacgaatgacagaggcgtgtataaggggctataatcctggactcttggtgcaccctgaccttgcctatttgcaagcagaaggtggaggggaccggcagctgggagatcgggaaaaagagctaatccgccaagcagctctgcagcagaccaaggagatggacctcagcgtggtgcggctcatgtttacagcttttcttccggatagcactggcagcttcacaaggcgcctggaacccgtggtatcagacgccatctatgacagtaaagcccccaatgcatccaacttgaaaattgtaagaatggacaggacagctggatgtgtgactggaggggaggaaatttatcttctttgtgacaaagttcagaaagatgacatccagattcgattttatgaagaggaagaaaatggtggagtctgggaaggatttggagatttttcccccacagatgttcatagacaatttgccattgtcttcaaaactccaaagtataaagatattaatattacaaaaccagcctctgtgtttgtccagcttcggaggaaatctgacttggaaactagtgaaccaaaacctttcctctactatcctgaaatcaaagataaagaagaagtgcagaggaaacgtcagaagctcatgcccaatttttcggatagtttcggcggtggtagtggtgctggagctggaggcggaggcatgtttggtagtggcggtggaggagggggcactggaagtacaggtccagggtatagcttcccacactatggatttcctacttatggtgggattactttccatcctggaactactaaatctaatgctgggatgaagcatggaaccatggacactgaatctaaaaaggaccctgaaggttgtgacaaaagtgatgacaaaaacactgtaaacctctttgggaaagttattgaaaccacagagcaagatcaggagcccagcgaggccaccgttgggaatggtgaggtcactctaacgtatgcaacaggaacaaaagaagagagtgctggagttcaggataacctctttctagagaaggctatgcagcttgcaaagaggcatgccaatgcccttttcgactacgcggtgacaggagacgtgaagatgctgctggccgtccagcgccatctcactgctgtgcaggatgagaatggggacagtgtcttacacttagcaatcatccaccttcattctcaacttgtgagggatctactagaagtcacatctggtttgatttctgatgacattatcaacatgagaaatgatctgtaccagacgcccttgcacttggcagtgatcactaagcaggaagatgtggtggaggatttgctgagggctggggccgacctgagccttctggaccgcttgggtaactctgttttgcacctagctgccaaagaaggacatgataaagttctcagtatcttactcaagcacaaaaaggcagcactacttcttgaccaccccaacggggacggtctgaatgccattcatctagccatgatgagcaatagcctgccatgtttgctgctgctggtggccgctggggctgacgtcaatgctcaggagcagaagtccgggcgcacagcactgcacctggctgtggagcacgacaacatctcattggcaggctgcctgctcctggagggtgatgcccatgtggacagtactacctacgatggaaccacacccctgcatatagcagctgggagagggtccaccaggctggcagctcttctcaaagcagcaggagcagatcccctggtggagaactttgagcctctctatgacctggatgactcttgggaaaatgcaggagaggatgaaggagttgtgcctggaaccacgcctctagatatggccaccagctggcaggtatttgacatattaaatgggaaaccatatgagccagagtttacatctgatgatttactagcacaaggagacatgaaacagctggctgaagatgtgaagctgcagctgtataagttactagaaattcctgatccagacaaaaactgggctactctggcgcagaaattaggtctggggatacttaataatgccttccggctgagtcctgctccttccaaaacacttatggacaactatgaggtctctgggggtacagtcagagagctggtggaggccctgagacaaatgggctacaccgaagcaattgaagtgatccaggcagcctccagcccagtgaagaccacctctcaggcccactcgctgcctctctcgcctgcctccacaaggcagcaaatagacgagctccgagacagtgacagtgtctgcgacagcggcgtggagacatccttccgcaaactcagctttaccgagtctctgaccagtggtgcctcactgctaactctcaacaaaatgccccatgattatgggcaggaaggacctctagaaggcaaaatttag

SEQ ID NO: 28 - DNA, 2844 bp, Homo sapiens
atggcagtgatggaaatggcctgcccaggtgcccctggctcagcagtggggcagcagaaggaactccccaaagccaaggagaagacgccgccactggggaagaaacagagctccgtctacaagcttgaggccgtggagaagagccctgtgttctgcggaaagtgggagatcctgaatgacgtgattaccaagggcacagccaaggaaggctccgaggcagggccagctgccatctctatcatcgcccaggctgagtgtgagaatagccaagagttcagccccaccttttcagaacgcattttcatcgctgggtccaaacagtacagccagtccgagagtcttgatcagatccccaacaatgtggcccatgctacagagggcaaaatggcccgtgtgtgttggaagggaaagcgtcgcagcaaagcccggaagaaacggaagaagaagagctcaaagtccctggctcatgcaggagtggccttggccaaacccctccccaggacccctgagcaggagagctgcaccatcccagtgcaggaggatgagtctccactcggcgccccatatgttagaaacaccccgcagttcaccaagcctctgaaggaaccaggccttgggcaactctgttttaagcagcttggcgagggcctacggccggctctgcctcgatcagaactccacaaactgatcagccccttgcaatgtctgaaccacgtgtggaaactgcaccacccccaggacggaggccccctgcccctgcccacgcaccccttcccctatagcagactgcctcatcccttcccattccaccctctccagccctggaaacctcaccctctggagtccttcctgggcaaactggcctgtgtagacagccagaaacccttgcctgacccacacctgagcaaactggcctgtgtagacagtccaaagcccctgcctggcccacacctggagcccagctgcctgtctcgtggtgcccatgagaagttttctgtggaggaatacctagtgcatgctctgcaaggcagcgtgagctcaggccaggcccacagcctgaccagcctggccaagacctgggcagcaaggggctccagatcccgggagcccagccccaaaactgaggacaacgagggtgtcctgctcactgagaaactcaagccagtggattatgagtaccgagaagaagtccactgggccacgcaccagctccgcctgggcagaggctccttcggagaggtgcacaggatggaggacaagcagactggcttccagtgcgctgtcaaaaaggtgcggctggaagtatttcgggcagaggagctgatggcatgtgcaggattgacctcacccagaattgtccctttgtatggagctgtgagagaagggccttgggtcaacatcttcatggagctgctggaaggtggctccctgggccagctggtcaaggagcagggctgtctcccagaggaccgggccctgtactacctgggccaggccctggagggtctggaatacctccactcacgaaggattctgcatggggacgtcaaagctgacaacgtgctcctgtccagcgatgggagccacgcagccctctgtgactttggccatgctgtgtgtcttcaacctgatggcctgggaaagtccttgctcacaggggactacatccctggcacagagacccacatggctccggaggtggtgctgggcaggagctgcgacgccaaggtggatgtctggagcagctgctgtatgatgctgcacatgctcaacggctgccacccctggactcagttcttccgagggccgctctgcctcaagattgccagcgagcctccgcctgtgagggagatcccaccctcctgcgcccctctcacagcccaggccatccaagaggggctgaggaaagagcccatccaccgcgtgtctgcagcggagctgggagggaaggtgaaccgggcactacagcaagtgggaggtctgaagagcccttggaggggagaatataaagaaccaagacatccaccgccaaatcaagccaattaccaccagaccctccatgcccagccgagagagctttcgccaagggccccagggccccggccagctgaggagacaacaggcagagcccctaagctccagcctcctctcccaccagagcccccagagccaaacaagtctcctcccttgactttgagcaaggaggagtctgggatgtgggaacccttacctctgtcctccctggagccagcccctgccagaaaccccagctcaccagagcggaaagcaaccgtcccggagcaggaactgcagcagctggaaatagaattattcctcaacagcctgtcccagccattttctctggaggagcaggagcaaattctctcgtgcctcagcatcgacagcctctccctgtcggatgacagtgagaagaacccatcaaaggcctctcaaagctcgcgggacaccctgagctcaggcgtacactcctggagcagccaggccgaggctcgaagctccagctggaacatggtgctggcccgggggcggcccaccgacaccccaagctatttcaatggtgtgaaagtccaaatacagtctcttaatggtgaacacctgcacatccgggagttccaccgggtcaaagtgggagacatcgccactggcatcagcagccagatcccagctgcagccttcagcttggtcaccaaagacgggcagcctgttcgctacgacatggaggtgccagactcgggcatcgacctgcagtgcacactggcccctgatggcagcttcgcctggagctggagggtcaagcatggccagctggagaacaggccctaa

SEQ ID NO: 29 - DNA, 1083 bp, Homo sapiens
atgtctcaggagaggcccacgttctaccggcaggagctgaacaagacaatctgggaggtgcccgagcgttaccagaacctgtctccagtgggctctggcgcctatggctctgtgtgtgctgcttttgacacaaaaacggggttacgtgtggcagtgaagaagctctccagaccatttcagtccatcattcatgcgaaaagaacctacagagaactgcggttacttaaacatatgaaacatgaaaatgtgattggtctgttggacgtttttacacctgcaaggtctctggaggaattcaatgatgtgtatctggtgacccatctcatgggggcagatctgaacaacattgtgaaatgtcagaagcttacagatgaccatgttcagttccttatctaccaaattctccgaggtctaaagtatatacattcagctgacataattcacagggacctaaaacctagtaatctagctgtgaatgaagactgtgagctgaagattctggattttggactggctcggcacacagatgatgaaatgacaggctacgtggccactaggtggtacagggctcctgagatcatgctgaactggatgcattacaaccagacagttgatatttggtcagtgggatgcataatggccgagctgttgactggaagaacattgtttcctggtacagaccatattaaccagcttcagcagattatgcgtctgacaggaacaccccccgcttatctcattaacaggatgccaagccatgaggcaagaaactatattcagtctttgactcagatgccgaagatgaactttgcgaatgtatttattggtgccaatcccctggctgtcgacttgctggagaagatgcttgtattggactcagataagagaattacagcggcccaagcccttgcacatgcctactttgctcagtaccacgatcctgatgatgaaccagtggccgatccttatgatcagtcctttgaaagcagggacctccttatagatgagtggaaaagcctgacctatgatgaagtcatcagctttgtgccaccaccccttgaccaagaagagatggagtcctga

SEQ ID NO: 30 - DNA, 1656 bp, Homo sapiens
atggctggtgatctttcagcaggtttcttcatggaggaacttaatacataccgtcagaagcagggagtagtacttaaatatcaagaactgcctaattcaggacctccacatgataggaggtttacatttcaagttataatagatggaagagaatttccagaaggtgaaggtagatcaaagaaggaagcaaaaaatgccgcagccaaattagctgttgagatacttaataaggaaaagaaggcagttagtcctttattattgacaacaacgaattcttcagaaggattatccatggggaattacataggccttatcaatagaattgcccagaagaaaagactaactgtaaattatgaacagtgtgcatcgggggtgcatgggccagaaggatttcattataaatgcaaaatgggacagaaagaatatagtattggtacaggttctactaaacaggaagcaaaacaattggccgctaaacttgcatatcttcagatattatcagaagaaacctcagtgaaatctgactacctgtcctctggttcttttgctactacgtgtgagtcccaaagcaactctttagtgaccagcacactcgcttctgaatcatcatctgaaggtgacttctcagcagatacatcagagataaattctaacagtgacagtttaaacagttcttcgttgcttatgaatggtctcagaaataatcaaaggaaggcaaaaagatctttggcacccagatttgaccttcctgacatgaaagaaacaaagtatactgtggacaagaggtttggcatggattttaaagaaatagaattaattggctcaggtggatttggccaagttttcaaagcaaaacacagaattgacggaaagacttacgttattaaacgtgttaaatataataacgagaaggcggagcgtgaagtaaaagcattggcaaaacttgatcatgtaaatattgttcactacaatggctgttgggatggatttgattatgatcctgagaccagtgatgattctcttgagagcagtgattatgatcctgagaacagcaaaaatagttcaaggtcaaagactaagtgccttttcatccaaatggaattctgtgataaagggaccttggaacaatggattgaaaaaagaagaggcgagaaactagacaaagttttggctttggaactctttgaacaaataacaaaaggggtggattatatacattcaaaaaaattaattcatagagatcttaagccaagtaatatattcttagtagatacaaaacaagtaaagattggagactttggacttgtaacatctctgaaaaatgatggaaagcgaacaaggagtaagggaactttgcgatacatgagcccagaacagatttcttcgcaagactatggaaaggaagtggacctctacgctttggggctaattcttgctgaacttcttcatgtatgtgacactgcttttgaaacatcaaagtttttcacagacctacgggatggcatcatctcagatatatttgataaaaaagaaaaaactcttctacagaaattactctcaaagaaacctgaggatcgacctaacacatctgaaatactaaggaccttgactgtgtggaagaaaagcccagagaaaaatgaacgacacacatgttag

SEQ ID NO: 31 - DNA, 1443 bp, Homo sapiens
atgagcgacgtggctattgtgaaggagggttggctgcacaaacgaggggagtacatcaagacctggcggccacgctacttcctcctcaagaatgatggcaccttcattggctacaaggagcggccgcaggatgtggaccaacgtgaggctcccctcaacaacttctctgtggcgcagtgccagctgatgaagacggagcggccccggcccaacaccttcatcatccgctgcctgcagtggaccactgtcatcgaacgcaccttccatgtggagactcctgaggagcgggaggagtggacaaccgccatccagactgtggctgacggcctcaagaagcaggaggaggaggagatggacttccggtcgggctcacccagtgacaactcaggggctgaagagatggaggtgtccctggccaagcccaagcaccgcgtgaccatgaacgagtttgagtacctgaagctgctgggcaagggcactttcggcaaggtgatcctggtgaaggagaaggccacaggccgctactacgccatgaagatcctcaagaaggaagtcatcgtggccaaggacgaggtggcccacacactcaccgagaaccgcgtcctgcagaactccaggcaccccttcctcacagccctgaagtactctttccagacccacgaccgcctctgctttgtcatggagtacgccaacgggggcgagctgttcttccacctgtcccgggagcgtgtgttctccgaggaccgggcccgcttctatggcgctgagattgtgtcagccctggactacctgcactcggagaagaacgtggtgtaccgggacctcaagctggagaacctcatgctggacaaggacgggcacattaagatcacagacttcgggctgtgcaaggaggggatcaaggacggtgccaccatgaagaccttttgcggcacacctgagtacctggcccccgaggtgctggaggacaatgactacggccgtgcagtggactggtgggggctgggcgtggtcatgtacgagatgatgtgcggtcgcctgcccttctacaaccaggaccatgagaagctttttgagctcatcctcatggaggagatccgcttcccgcgcacgcttggtcccgaggccaagtccttgctttcagggctgctcaagaaggaccccaagcagaggcttggcgggggctccgaggacgccaaggagatcatgcagcatcgcttctttgccggtatcgtgtggcagcacgtgtacgagaagaagctcagcccacccttcaagccccaggtcacgtcggagactgacaccaggtattttgatgaggagttcacggcccagatgatcaccatcacaccacctgaccaagatgacagcatggagtgtgtggacagcgagcgcaggccccacttcccccagttctcctactcggccagcggcacggcctga

SEQ ID NO: 32 - DNA, 2514 bp, Homo sapiens
atggctagcaaacgaaaatctacaactccatgcatggttcggacatcacaagtagtagaacaagatgtgcccgaggaagtagacagggccaaagagaaaggaatcggcacaccacagcctgacgtggccaaggacagttgggcagcagaacttgaaaactcttccaaagaaaacgaagtgatagaggtgaaatctatgggggaaagccagtccaaaaaactccaaggtggttatgagtgcaaatactgcccctactccacgcaaaacctgaacgagttcacggagcatgtcgacatgcagcatcccaacgtgattctcaaccccctctacgtgtgtgcagaatgtaacttcacaaccaaaaagtacgactccctatccgaccacaactccaagttccatcccggggaggccaacttcaagctgaagttaattaaacgcaataatcaaactgtcttggaacagtccatcgaaaccaccaaccatgtcgtgtccatcaccaccagtggccctggaactggtgacagtgattctgggatctcggtgagtaaaacccccatcatgaagcctggaaaaccaaaagcggatgccaagaaggtgcccaagaagcccgaggagatcacccccgagaaccacgtggaagggaccgcccgcctggtgacagacacagctgagatcctctcgagactcggcggggtggagctcctccaagacacattaggacacgtcatgccttctgtacagctgccaccaaatatcaaccttgtgcccaaggtccctgtcccactaaatactaccaaatacaactctgccctggatacaaatgccacgatgatcaactctttcaacaagtttccttacccgacccaggctgagttgtcctggctgacagctgcctccaaacacccagaggagcacatcagaatctggtttgccacccagcgcttaaagcatggcatcagctggtccccagaagaggtggaggaggcccggaagaagatgttcaacggcaccatccagtcagtacccccgaccatcactgtgctgcccgcccagttggcccccacaaaggtgacgcagcccatcctccagacggctctaccgtgccagatcctcggccagactagcctggtgctgactcaggtgaccagcgggtcaacaaccgtctcttgctcccccatcacacttgccgtggcaggagtcaccaaccatggccagaagagacccttggtgactccccaagctgcccccgaacccaagcgtccacacatcgctcaggtgccagagcccccacccaaggtggccaaccccccgctcacaccagccagtgaccgcaagaagacaaaggagcagatagcacatctcaaggccagctttctccagagccagttccctgacgatgccgaggtttaccggctcatcgaggtgactggccttgccaggagcgagatcaagaagtggttcagtgaccaccgatatcggtgtcaaaggggcatcgtccacatcaccagcgaatcccttgccaaagaccagttggccatcgcggcctcccgacacggtcgcacgtatcatgcgtacccagactttgccccccagaagttcaaagagaaaacacagggtcaggttaaaatcttggaagacagctttttgaaaagttcttttcctacccaagcagaactggatcggctaagggtggagaccaagctgagcaggagagagatcgactcctggttctcggagaggcggaagcttcgagacagcatggaacaagctgtcttggattccatggggtctggcaaaaaaggccaagatgtgggagcccccaatggtgctctgtctcgactcgaccagctctccggtgcccagttaacaagttctctgcccagcccttcgccagcaattgcaaaaagtcaagaacaggttcatctcctgaggagcacgtttgcaagaacccagtggcctactccccaggagtacgaccagttagcggccaagactggcctggtccgaactgagattgtgcgttggttcaaggagaacagatgcttgctgaaaacgggaaccgtgaagtggatggagcagtaccagcaccagcccatggcagatgatcacggctacgatgccgtagcaaggaaagcaacaaaacccatggccgagagcccaaagaacgggggtgatgtggttccacaatattacaaggaccccaaaaagctctgcgaagaggacttggagaagttggtgaccagggtaaaagtaggcagcgagccagcaaaagactgtttgccagcaaagccctcagaggccacctcagaccggtcagagggcagcagccgggacggccagggtagcgacgagaacgaggagtcgagcgttgtggattacgtggaggtgacggtcggggaggaggatgcgatctcagatagatcagatagctggagtcaggctgcggcagaaggtgtgtcggaactggctgaatcagactccgactgcgtccctgcagaggctggccaggcctag

SEQ ID NO: 33 - DNA, 567 bp, Homo sapiens
atgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgtaa

SEQ ID NO: 34 - DNA, 570 bp, Homo sapiens
atgactgagtacaaactggtggtggttggagcaggtggtgttgggaaaagcgcactgacaatccagctaatccagaaccactttgtagatgaatatgatcccaccatagaggattcttacagaaaacaagtggttatagatggtgaaacctgtttgttggacatactggatacagctggacaagaagagtacagtgccatgagagaccaatacatgaggacaggcgaaggcttcctctgtgtatttgccatcaataatagcaagtcatttgcggatattaacctctacagggagcagattaagcgagtaaaagactcggatgatgtacctatggtgctagtgggaaacaagtgtgatttgccaacaaggacagttgatacaaaacaagcccacgaactggccaagagttacgggattccattcattgaaacctcagccaagaccagacagggtgttgaagatgctttttacacactggtaagagaaatacgccagtaccgaatgaaaaaactcaacagcagtgatgatgggactcagggttgtatgggattgccatgtgtggtgatgtaa

SEQ ID NO: 35 - DNA, 2016 bp, Homo sapiens
atgcaaccagacatgtccttgaatgtcattaagatgaaatccagtgacttcctggagagtgcagaactggacagcggaggctttgggaaggtgtctctgtgtttccacagaacccagggactcatgatcatgaaaacagtgtacaaggggcccaactgcattgagcacaacgaggccctcttggaggaggcgaagatgatgaacagactgagacacagccgggtggtgaagctcctgggcgtcatcatagaggaagggaagtactccctggtgatggagtacatggagaagggcaacctgatgcacgtgctgaaagccgagatgagtactccgctttctgtaaaaggaaggataattttggaaatcattgaaggaatgtgctacttacatggaaaaggcgtgatacacaaggacctgaagcctgaaaatatccttgttgataatgacttccacattaagatcgcagacctcggccttgcctcctttaagatgtggagcaaactgaataatgaagagcacaatgagctgagggaagtggacggcaccgctaagaagaatggcggcaccctctactacatggcgcccgagcacctgaatgacgtcaacgcaaagcccacagagaagtcggatgtgtacagctttgctgtagtactctgggcgatatttgcaaataaggagccatatgaaaatgctatctgtgagcagcagttgataatgtgcataaaatctgggaacaggccagatgtggatgacatcactgagtactgcccaagagaaattatcagtctcatgaagctctgctgggaagcgaatccggaagctcggccgacatttcctggcattgaagaaaaatttaggcctttttatttaagtcaattagaagaaagtgtagaagaggacgtgaagagtttaaagaaagagtattcaaacgaaaatgcagttgtgaagagaatgcagtctcttcaacttgattgtgtggcagtaccttcaagccggtcaaattcagccacagaacagcctggttcactgcacagttcccagggacttgggatgggtcctgtggaggagtcctggtttgctccttccctggagcacccacaagaagagaatgagcccagcctgcagagtaaactccaagacgaagccaactaccatctttatggcagccgcatggacaggcagacgaaacagcagcccagacagaatgtggcttacaacagagaggaggaaaggagacgcagggtctcccatgacccttttgcacagcaaagaccttacgagaattttcagaatacagagggaaaaggcactgcttattccagtgcagccagtcatggtaatgcagtgcaccagccctcagggctcaccagccaacctcaagtactgtatcagaacaatggattatatagctcacatggctttggaacaagaccactggatccaggaacagcaggtcccagagtttggtacaggccaattccaagtcatatgcctagtctgcataatatcccagtgcctgagaccaactatctaggaaatacacccaccatgccattcagctccttgccaccaacagatgaatctataaaatataccatatacaatagtactggcattcagattggagcctacaattatatggagattggtgggacgagttcatcactactagacagcacaaatacgaacttcaaagaagagccagctgctaagtaccaagctatctttgataataccactagtctgacggataaacacctggacccaatcagggaaaatctgggaaagcactggaaaaactgtgcccgtaaactgggcttcacacagtctcagattgatgaaattgaccatgactatgagcgagatggactgaaagaaaaggtttaccagatgctccaaaagtgggtgatgagggaaggcataaagggagccacggtggggaagctggcccaggcgctccaccagtgttccaggatcgaccttctgagcagcttgatttacgtcagccagaactaa

SEQ ID NO: 36 - DNA, 1569 bp, Homo sapiens
atgagtctgctaaactgtgaaaacagctgtggatccagccagtctgaaagtgactgctgtgtggccatggccagctcctgtagcgctgtaacaaaagatgatagtgtgggtggaactgccagcacggggaacctctccagctcatttatggaggagatccagggatatgatgtagagtttgacccacccctggaaagcaagtatgaatgccccatctgcttgatggcattacgagaagcagtgcaaacgccatgcggccataggttctgcaaagcctgcatcataaaatcaataagggatgcaggtcacaaatgtccagttgacaatgaaatactgctggaaaatcaactatttccagacaattttgcaaaacgtgagattctttctctgatggtgaaatgtccaaatgaaggttgtttgcacaagatggaactgagacatcttgaggatcatcaagcacattgtgagtttgctcttatggattgtccccaatgccagcgtcccttccaaaaattccatattaatattcacattctgaaggattgtccaaggagacaggtttcttgtgacaactgtgctgcatcaatggcatttgaagataaagagatccatgaccagaactgtcctttggcaaatgtcatctgtgaatactgcaatactatactcatcagagaacagatgcctaatcattatgatctagactgccctacagccccaattccatgcacattcagtacttttggttgccatgaaaagatgcagaggaatcacttggcacgccacctacaagagaacacccagtcacacatgagaatgttggcccaggctgttcatagtttgagcgttatacccgactctgggtatatctcagaggtccggaatttccaggaaactattcaccagttagagggtcgccttgtaagacaagaccatcaaatccgggagctgactgctaaaatggaaactcagagtatgtatgtaagtgagctcaaacgaaccattcgaacccttgaggacaaagttgctgaaatcgaagcacagcagtgcaatggaatttatatttggaagattggcaactttggaatgcatttgaaatgtcaagaagaggagaaacctgttgtgattcatagccctggattctacactggcaaacccgggtacaaactgtgcatgcgcttgcaccttcagttaccgactgctcagcgctgtgcaaactatatatccctttttgtccacacaatgcaaggagaatatgacagccacctcccttggcccttccagggtacaatacgccttacaattcttgatcagtctgaagcacctgtaaggcaaaaccacgaagagataatggatgccaaaccagagctgcttgctttccagcgacccacaatcccacggaacccaaaaggttttggctatgtaacttttatgcatctggaagccctaagacaaagaactttcattaaggatgacacattattagtgcgctgtgaggtctccacccgctttgacatgggtagccttcggagggagggttttcagccacgaagtactgatgcaggggtatag

SEQ ID NO: 37 - DNA, 999 bp, Homo sapiens
atggccaaccgttacaccatggatctgactgccatctacgagagcctcctgtcgctgagccctgacgtgcccgtgccatccgaccatggagggactgagtccagcccaggctggggctcctcgggaccctggagcctgagcccctccgactccagcccgtctggggtcacctcccgcctgcctggccgctccaccagcctagtggagggccgcagctgtggctgggtgcccccaccccctggcttcgcaccgctggctccccgcctgggccctgagctgtcaccctcacccacttcgcccactgcaacctccaccaccccctcgcgctacaagactgagctatgtcggaccttctcagagagtgggcgctgccgctacggggccaagtgccagtttgcccatggcctgggcgagctgcgccaggccaatcgccaccccaaatacaagacggaactctgtcacaagttctacctccagggccgctgcccctacggctctcgctgccacttcatccacaaccctagcgaagacctggcggccccgggccaccctcctgtgcttcgccagagcatcagcttctccggcctgccctctggccgccggacctcaccaccaccaccaggcctggccggcccttccctgtcctccagctccttctcgccctccagctccccaccaccacctggggaccttccactgtcaccctctgccttctctgctgcccctggcacccccctggctcgaagagaccccaccccagtctgttgcccctcctgccgaagggccactcctatcagcgtctgggggcccttgggtggcctggttcggaccccctctgtacagtccctgggatccgaccctgatgaatatgccagcagcggcagcagcctggggggctctgactctcccgtcttcgaggcgggagtttttgcaccaccccagcccgtggcagccccccggcgactccccatcttcaatcgcatctctgtttctgagtga